(19) United States
(12) Patent Application Publication
(10) Pub. No.: US 2024/0226271 A1
(43) Pub. Date: Jul. 11, 2024

(54) MODIFIED CORONAVIRUS STRUCTURAL PROTEIN

(71) Applicant: MEDICAGO INC., Quebec (CA)

(72) Inventors: Pierre-Olivier LAVOIE, Quebec (CA); Marc-Andre D'AOUST, Quebec (CA)

(21) Appl. No.: 18/024,140

(22) PCT Filed: Aug. 31, 2021
(86) PCT No.: PCT/CA2021/051201

§ 371 Date: Mar. 1, 2023

Related U.S. Application Data
(60) Provisional application No. 63/073,327, filed on Sep. 1, 2020.
(60) Provisional application No. 63/211,716, filed on Jun. 17, 2021.

(51) Int. Cl. A61K 39/215; C07K 14/005; C12N 7/00

(52) U.S. Cl. CPC A61K 39/215; C07K 14/005; C12N 7/00; C07K 2319/03; C12N 2760/16122; C12N 2770/20022; C12N 2770/20023; C12N 2770/20034; C12N 2770/20051

(57) ABSTRACT

Modified coronaviras spike (S)-protein, virus-like particle (VLPs) comprising the modified S protein and nucleic acids encoding modified S protein are provided. Methods for modified S-protein and VLP production in a host or host cell are also described. The modified S-protein may comprise a transmembrane domain (TM) or portion of a TM, and a cytosolic tail (CT) or of a CT, wherein the CT or portion of the CT is derived from an influenza hemagglutinin (HA) protein and wherein the TM or portion of the TM is heterologous to the CT or portion of the CT. In addition a method for inducing immunity to a coronaviras infection in a subject, comprising administering a composition comprising the modified coronaviras (S)-protein or VLP to the subject is described.

The contents of the electronic sequence listing submitted herewith as file 18636_0020U1_Sequence_Listing.txt; Size: 854,569 bytes; and Date of Creation: Jul. 17, 2023, is herein incorporated by reference in its entirety.

### FIELD OF INVENTION

The present disclosure relates to modified viral structural protein. The present invention also relates to virus-like particles (VLPs) comprising modified viral structural protein and methods of producing the VLPs in a host or host cells.

### BACKGROUND

Coronaviruses (CoVs) are the largest group of viruses belonging to the Nidovirales order, which includes Coronaviridae, Arteriviridae, Mesoniviridae, and Roniviridae families. The Coronavirinae comprise one of two subfamilies in the Coronaviridae family, with the other being the Torovirinae. The Coronavirinae are further subdivided into four genera, the alpha, beta, gamma, and delta coronaviruses. Members of alpha coronavirus and beta coronavirus are found exclusively in mammals. The alphacoronavirus genus includes two human virus species, HCoV-229E and HCoV-NL63. Important animal alphacoronaviruses are transmissible gastroenteritis virus of pigs and feline infectious peritonitis virus.

Severe acute respiratory syndrome coronavirus 2 (SARS-CoV-2, also known as 2019-nCoV and HCoV-19) is a novel lineage B betacoronavirus (Beta-CoV) and causes coronavirus disease 2019 (COVID-19), a respiratory illness with high mortality and morbidity resulting in major public health impacts worldwide. Outbreaks of SARS-CoV-2, such as the pandemic starting in 2020, are a paramount challenge for healthcare systems due to the incubation period and transmissibility of the virus. Treatments for COVID-19 are urgently needed, but long-term management of SARS-CoV-2 outbreaks will require an effective vaccine.

Coronavirus virions are spherical with diameters of approximately 118-140 nm as depicted in recent studies by cryo-electron tomography and cryo-electron microscopy.

The most prominent structural feature of coronaviruses is the club-shaped spike projections emanating from the surface of the virion. Coronavirus particles consist of a helical nucleocapsid structure, formed by the association between nucleocapsid (N) phosphoproteins and the viral genomic RNA, surrounded by a lipid bilayer where three or four types of structural proteins are inserted: the spike (S), the membrane (M), and the envelope (E) proteins and, for some coronaviruses only, the hemagglutinin-esterase (HE) protein (Masters PS. The molecular biology of coronaviruses. Adv Virus Res. 2006; 66:193-292.)

The membrane (M) protein is the most abundant structural protein in the virion. It is a small (˜25-30 kDa) protein with three transmembrane domains and is thought to give the virion its shape. The envelope (E) protein is a short, integral membrane protein of 76-109 amino acids, ranging from 8.4 to 12 kDa in size. The primary and secondary structure reveals that E has a short, hydrophilic amino terminus consisting of 7-12 amino acids, followed by a large hydrophobic transmembrane domain (TMD) of 25 amino acids, and ends with a long, hydrophilic carboxyl terminus, which comprises the majority of the protein. The E protein is involved in several aspects of the virus' life cycle, such as assembly, budding, envelope formation, and pathogenesis.

The spike (S) protein is a glycoprotein that is required for the recognition of host receptors for many coronaviruses as well as the fusion of viral and host cell membranes for viral entry into cells (Belouzard et al., Viruses 2012 June; 4(6):1011-33). As the primary glycoprotein on the surface of the viral envelope, S proteins of Coronaviridae are a major target of neutralizing antibodies elicited by natural infection, including SARS-CoV-2 infection, and are key antigens targeted in experimental vaccine candidates.

SARS-CoV-2 S protein, like S protein of other coronaviruses, is initially synthesized as a precursor protein. Individual precursor S protein forms a homotrimer and undergoes glycosylation within the Golgi compartment as well as processing to remove the signal peptide. The S protein requires a two-step, protease-mediated activation to facilitate membrane fusion. SARS-CoV-2 S protein is distinguished by a RRAR furin cleavage site at the S1/S2 junction that is presumably processed in the Golgi compartment to yield two separate polypeptides: the S1 and S2 polypeptide (or subunit), which remain non-covalently bound as S1/S2 protomers within the homotrimer in the prefusion conformation (Walls et al. Cell 2020 181(2) p 281-292; Li et al. eLife 2019; 8: e51230.). Furin cleavage at the S1/S2 junction and cleavage at the S2′ site, upstream of the fusion peptide, occurs during viral entry at the cell surface or in endosomes and can be mediated by several proteases.

This trimer is held in the prefusion conformation prior to binding to target receptors on a host cell via receptor binding domain (RBD) epitopes. Receptor binding destabilizes the prefusion trimer, resulting in shedding of the S1 subunit and transition of the S2 subunit to a stable post-fusion conformation through fusion of the virus to the cell membrane (Wrapp et al. Science, 13 Mar. 2020, Vol. 367, Issue 6483, pp. 1260-1263). Neutralizing antibodies from individuals infected with SARS-CoV-2 have been shown to target the RBD of the S1 subunit of the S protein (Premkumar, L., 2020 Science Immunology 11 Jun. 2020: Vol. 5, Issue 48).

Stabilization of the S protein ectodomain in the prefusion conformation tends to increase the recombinant expression yield, possibly by preventing triggering or misfolding that results from a tendency to adopt the more stable post-fusion structure (Hsieh et al. Science 2020, 369 p. 1501-1505).

Mutations to the S protein ectodomain have been shown to facilitate stabilization of the prefusion conformation. WO 2018/081318 and its companion publication by Pallesen, J. et al. (PNAS Aug. 29, 2017 114 (35)) disclose double proline substitutions at or near a junction between a heptad repeat 1 (HR1) and a central helix that stabilize the S ectodomain trimer of MERS-CoV spike protein in a prefusion conformation and substitutions to prevent protease cleavage at a S1/S2 cleavage site and the S2′ cleavage site of the S ectodomain. SARS-CoV-2 S protein stabilized with double proline substitutions at homologous amino acid residues have been used to determine high-resolution structures by cryo-EM (Wrapp et al Science 2020 367, 1260-1263; Walls et al. Cell 2020, 181, 281-292). Further, disruption of the furin recognition site is thought to retain S protein in a prefusion conformation (Wrapp et al Science 2020 367, 1260-1263). However, even with these substitutions, the SARS-CoV-2 S protein ectodomain remains unstable and difficult to produce reliably in mammalian cells, hindering development of effective and high-yield subunit vaccines (Hsieh et al. Science 2020, 369 p. 1501-1505).

Hsieh et al. (Science 2020, 369 p. 1501-1505) designed and expressed in mammalian cells over 100 structure-guided SARS-CoV-2 spike protein variants based upon previously determined cryo-EM structure. The variants were biochemically, biophysically and structurally characterized to identify substitutions that lead to an increase in yield and stability. Hsieh et al. reports multiple prolines, disulfide bonds, salt bridges, and cavity-filling substitutions that increase expression and/or stability of the spike relative to the double proline substitutions. The best identified variant, HexaPro, has six beneficial proline substitutions leading to 10-fold higher expression than its parental construct and is able to withstand heat stress, storage at room temperature, and multiple freeze-thaws.

The S2 subunit can be divided into three domains: a large ectodomain, a transmembrane domain (TM) and a cytoplasmic tail (CT). The cytoplasmic tail of the S protein has previously been shown to be required for assembly. Two distinct retention signals may be found in the CT of Coronaviridae: i) an endoplasmic reticulum retrieval signal (ERRS) and/or ii) a tyrosine-dependent localization signal (YxxI or YxxF motif). The ERRS comprises the dibasic KxHxx motif which binds to the coatomer complex I (COPI). The motif is required for the localization of the SARS S protein to the ERGIC/Golgi region when coexpressed with SARS membrane (M) protein, and localization can be disrupted by mutating the KxHxx motif (McBride et al. J. Virol. February 2007, 81 (5) 2418-2428). S proteins containing an ERRS are recruited into COPI vesicles and retrieved from the Golgi to the endoplasmic reticulum (ER) in retrograde. The repeated cycling of S proteins between the ER and the Golgi leads to S protein intracellular retention. S protein of Alphacoronavirus and Betacoronavirus both comprise an ERRS (Ujike et al. Journal of General Virology (2016), 97, 1853-1864).

S protein of Betacoronavirus, such as S protein of MERS-CoV, SARS-CoV and SARS-CoV 2, possess only an ERRS and cannot be retained intracellularly, resulting in the release of S protein into the plasma membrane. Mutant SARS-CoV S protein lacking the ERRS is transported to the plasma membrane, while native S protein, when coexpressed with M protein, interacts with the M protein near the budding site, leading to S protein intracellular retention, suggesting that the ERRS of SARS-CoV contributes to S protein accumulation specifically in the post-medial Golgi compartment by interaction with M protein, leading to S protein incorporation into VLPs (Ujike et al. Journal of General Virology (2016), 97, 1853-1864). Removal of the ERRS has recently been found to facilitate incorporation of SARS-CoV-2 S protein into lentiviral pseudovirons (Ou et al., 2020 Nature Communications volume 11, Article number: 1620).

Yu et al. (2020 Science) constructed a set of prototype DNA vaccines expressing six variants of the SARS-CoV-2 S protein with various deletions of the cytoplasmic tail, and transmembrane domain, which were assessed for their immunogenicity and protective efficacy against SARS-CoV-2 viral challenge in rhesus macaques. While the soluble fragments of the SARS-CoV-2 S protein ectodomain elicited reduced levels of sgmRNA (indicative of viral replication), optimal protection was achieved with the full-length S protein immunogen.

Broer et al. (2006 J. Virol. p. 1302-1310) studied the roles of the transmembrane and cytoplasmic domains of the S protein in the infectivity and membrane fusion activity of SARS-CoV, using a SARS-CoV S-pseudotyped retrovirus (SARSpp). SARSpp, in which the cytoplasmic domain of S was replaced by the cytoplasmic domain derived from vesicular stomatitis virus G protein (VSV-G), were infectious, up to 40% of wild type. In contrast, SARSpp containing both the TMD and the cytoplasmic domain of VSV-G, were severely impaired in infectivity (<5%). This shows that the TMD of S may be involved in the entry process of SARS-CoV.

Vaccination provides protection against disease by inducing a subject to mount an immune response to a likely agent prior to infection. Conventionally, this has been accomplished through the use of live attenuated or whole inactivated forms of the infectious agents as immunogens. To avoid the danger of using a whole virus (such as killed or attenuated viruses) as a vaccine, viral proteins or subunits, or recombinant versions thereof, have been pursued as vaccines. A major obstacle to employing viral proteins, either native or recombinant, as vaccine agents is ensuring that the conformation of the protein mimics the antigens in their natural environment. Suitable adjuvants and, in the case of peptides, carrier proteins, may be used to boost the immune response. In addition, viral proteins or subunits as vaccines may elicit primarily humoral responses and thus fail to evoke lasting immunity. Subunit vaccines may be ineffective for diseases in which whole inactivated virus can be demonstrated to provide superior protection.

Virus-like particles (VLPs) may be used in immunogenic compositions to express viral proteins in a preferred conformation with improved antigen presentation to the immune system. VLPs closely resemble mature virions, but they do not contain viral genomic material, and they are non-replicative which makes them safe for administration as a vaccine. In addition, VLPs can be engineered to express viral glycoproteins on the surface of the VLP, which is their native physiological configuration. Since VLPs resemble intact virions and are multivalent particulate structures, VLPs may be more effective in inducing neutralizing antibodies to the glycoprotein than soluble envelope protein antigens.

A variety of expression systems have been utilized to produce VLPs, including mammalian cell lines, bacteria, insect cell lines, yeast and plant cells. VLPs for over thirty different viruses have been generated in insect and mammalian systems for vaccine purposes (Noad, R. and Roy, P., 2003, Trends Microbiol 11: 438-44). VLPs have also been produced in plants (see WO2009/076778; WO2009/009876; WO 2009/076778; WO 2010/003225; WO 2010/003235; WO2010/006452; WO2011/03522; WO 2010/148511; WO2014153674, and WO2012/083445).

VLPs have been produced with native surface proteins from Severe acute respiratory syndrome coronavirus (SARS-CoV or SARS-CoV-1), including S protein, M protein, E protein in insect and mammalian cells (Liu et al., 2008, J Virol., p. 11318-11330). SARS-CoV-2 virus like particles (VLPs) have also been assembled by co-expressing viral surface proteins S, M, and E in mammalian cells (Xu et al. Front. Bioeng. Biotechnol., 30 Jul. 2020). Studies have further shown that the M protein is indispensable for virus-like particle (VLP) formation (Siu et al. Journal of Virology (2008) 82:11318-11330, Huang et al. Journal of Virology (2004) 78:12557-12565). In mammalian cells, expression of membrane protein (M) and small envelope protein (E) are essential for efficient formation and release of SARS-CoV-2 VLPs (Xu et al. Front. Bioeng. Biotechnol., 30 Jul. 2020)). Nevertheless, the minimal requirement for assembly of SARS-CoV VLPs is still controversial. Y. Huang et al. (Journal of Virology (2004) 78:12557-12565) described formation of VLPs in transfected human cells that only required co-expression of the M and N viral proteins, whereas Siu et al. (Journal of Virology (2008) 82:11318-11330) showed that both E and N proteins must be coexpressed with M protein for the efficient production and release of SARS-CoV VLPs in transfected mammalian cells.

WO2012/083445 discloses the production of SARS CoV S protein in plants, wherein the transmembrane domain and the cytosolic tail domain (TM/CT) of the S protein were replaced with TM/CT from an influenza HA protein.

A few groups have proposed immunization with SARS-CoV VLPs as an effective vaccine strategy. VLPs produced in insect cells or chimeric MHV/SARS-CoV VLPs produced in mammalian cells were used in these studies (Lokugamage et al. Vaccine 2008 Feb. 6; 26(6):797-808, Lu et al. 2007 Immunology 122496-5024).

However, effective scale-up and manufacture of SARS-CoV-2 VLPs at the quantity required to meet the need of widespread vaccination of the global population, requires efficient viral structural protein and VLP production.

### SUMMARY OF THE INVENTION

The present invention relates to modified viral structural proteins. The present invention also relates to virus-like particles (VLPs) comprising modified viral structural protein and methods of producing the VLPs in a host or host cells. More specifically, the invention relates to modified coronavirus S proteins. The present invention also relates to virus-like particles (VLPs) comprising modified S proteins and methods of producing the VLPs in a host or host cells.

In one aspect it is provided a modified coronavirus S-protein comprising, in series,
an ectodomain derived from a coronavirus S-protein,
a transmembrane and cytosolic tail domain (TMCT), wherein the TMCT is a chimeric TMCT, comprising:
   a transmembrane domain (TM), wherein the TM or a portion of the TM is derived from a coronavirus S-protein, and
   a cytosolic tail (CT), wherein the CT or a portion of the CT is derived from an influenza hemagglutinin (HA) protein.

The modified S-protein as described herein may form trimers. Accordingly it is also provided a trimer comprising modified coronavirus S-protein as described herewith.

In a further aspect, a virus like particle (VLP) comprising the modified S-protein or trimers comprising the modified S-protein as described above is provided. Accordingly, the VLP comprises a modified coronavirus S-protein or trimer that comprise the modified S-protein, the modified S-protein comprising
an ectodomain derived from a coronavirus S-protein,
a transmembrane and cytosolic tail domain (TMCT), wherein the TMCT is a chimeric TMCT, comprising:
   a transmembrane domain (TM), wherein the TM or a portion of the TM is derived from a coronavirus S-protein and
   a cytosolic tail (CT), wherein the CT or a portion of the CT is derived from an influenza hemagglutinin (HA) protein.

The VLP may further comprise plant lipids.

The TM may be directly fused to the CT. The TM may be derived from the coronavirus S-protein TM and the CT may be derived from the influenza HA protein CT. Furthermore, the TM may be a chimeric TM comprising a N terminal sequence derived from the coronavirus S-protein TM and a C terminal sequence derived from the influenza HA protein TM. The chimeric TM may comprise a N terminal sequence derived from the coronavirus S-protein TM comprising at least 20 amino acids corresponding to amino acids 1-20 of SEQ ID NO: 18 or SEQ ID NO: 169, or at least 21 amino acids corresponding to amino acids 1-21 of SEQ ID NO: 118 or 164, or at least 22 amino acids corresponding to amino acids 1-22 of SEQ ID NO: 123 and one or more than one amino acid from the C-terminal end of the influenza HA protein TM. The one or more than one amino acid from the C-terminal end of the influenza HA protein TM may be selected from AGL or conserved substitution of AGL, MAGL or conserved substitution of MAGL. The chimeric TM may comprise amino acids corresponding to amino acids of 1-20 of SEQ ID NO: 18.

The CT may be chimeric CT comprising a N terminal sequence derived from the coronavirus S-protein CT and a C terminal sequence derived from the influenza HA protein CT. The chimeric CT may comprise a C terminal sequence derived from influenza HA protein CT comprising at least 11 amino acids corresponding to amino acids 27-37 of SEQ ID NO: 18, 126, 127, 128, 129, 130 or 131 and one or more than one amino acid from the N-terminal end of the coronavirus S-protein CT. The one or more than one amino acid from the N-terminal end of the coronavirus S-protein CT may be selected from C or a conserved substitution of C, CC or a conserved substitution of CC, or CCM or a or a conserved substitution of CCM. The chimeric CT may comprise amino acids corresponding to amino acids of 27-37 of SEQ ID NO: 18, 126, 128, 129, 130 or 131; or amino acids 27-36 of SEQ ID NO: 127. In one aspect the chimeric TMCT may comprise a chimeric TM comprising amino acids corresponding to amino acids 1-20 of SEQ ID NO: 18 or SEQ ID NO: 169, or to amino acids 1-21 of SEQ ID NO: 118 or SEQ ID NO: 164, or amino acids 1-22 of SEQ ID NO: 123, a chimeric CT comprising amino acids corresponding to amino acids 27-37 of SEQ ID NO: 18, 126, 127, 128, 129, 130 or 131, or a combination thereof.

The CT or portion of the CT may comprise from 80% to 100% identity with the sequence of SEQ ID NO: 15, or with amino acids 35-50 of SEQ ID NO 6, 8, 7, 9, 10, 12, 13 or 14, or with amino acids 34-49 of SEQ ID NO 11, or with amino acids 553-568 of SEQ ID NO:3 or with amino acids 22-37 of SEQ ID NO:18, or with amino acids 21-40 of SEQ ID NO: 19, or with amino acids 21-39 of SEQ ID NO: 37, or with amino acids 25-36 of SEQ ID NO: 38 or with amino acids 24-34 of SEQ ID NO: 39, or amino acids 22-37 of SEQ ID NO: 126, 128, 129, 130 or 131; or amino acids 22-36 of SEQ ID NO: 127. The TM or portion of the TM may comprises from 80% to 100% identity with the sequence of SEQ ID NO: 132 or 133.

The TMCT may comprise a sequence having about 80% to about 100% identity with the sequence of SEQ ID NO: 18, 19, 37, 38, 39, 64, 126, 127, 128, 129, 130, 131, 118, 119, 120, 123, 124, 125, 134, 135 164, 165, 166, 169, 170, 171, 172 or 173.

The modified S protein may comprises an S1 subunit and an S2 subunit, wherein the S2 subunit comprises the chimeric TMCT.

The modified S-protein may be produced as a precursor protein, the precursor protein comprising the modified S-protein and a signal peptide. The precursor protein comprising the modified S-protein and a signal peptide may comprise from 80% to 100% identity with amino acids 1-1234 of SEQ ID 1, or with amino acids 1-1234 of SEQ ID NO: 5, amino acids 1-1219 of SEQ ID NO: 21 or with amino acids 1-1243 of SEQ ID NO: 30 and wherein the amino acid sequence of the CT comprises from 80% to 100% identity with the sequence of SEQ ID NO: 15, or with amino acids 35-50 of SEQ ID NO 6, 8, 7, 9, 10, 12, 13 or 14, or with amino acids 34-49 of SEQ ID NO 11, or with amino acids 553-568 of SEQ ID NO:3.

The signal peptide may be native or non-native to the S-protein. The non-native signal peptide may be derived from the signal peptide of protein disulfide-isomerase (PDI). The modified S-protein may further comprise plant specific N-glycans.

The CT or portion of the CT in the modified S-protein may be derived from an influenza hemagglutinin (HA) protein that is derived from influenza type B or influenza subtype H1, H2, H3, H4, H5, H6, H7, H8, H9, H10, H11, H12, H13, H14, H15, or H16. The influenza hemagglutinin (HA) protein may be derived from influenza type B or influenza subtype H1, H3, H5, H6, H7 or H9.

The ectodomain of the modified S-protein may be derived from SARS-CoV-2, SARS-CoV-1, MERS-CoV, OC43-CoV or 229E-CoV, the TM or the portion of the TM may be derived from SARS-CoV-2, SARS-CoV-1, MERS-CoV, OC43-CoV or 229E-CoV, or both the ectodomain and the TM or the portion of the TM may be derived from SARS-CoV-2, SARS-CoV-1, MERS-CoV, OC43-CoV or 229E-CoV.

In a further aspect, the modified S-protein may comprise one or more than one amino acid substitution when compared to a wild-type coronavirus amino acid sequence. The one or more than one substitution may maintain the S-protein in a pre-fusion state.

The one or more than one amino acid substitution may comprise i) substitutions that restricts the processing at a cleavage site between S1 and S2 subunit, ii) substitution of one or more than one amino acid to one or more than one proline, or iii) substitutions that restrict the processing at the cleavage site between the S1 and the S2 subunit and substitution of one or more than one amino acid to one or more than one proline.

The one or more than one substitution may maintain the S-protein in a pre-fusion state or produces are higher yield of the modified S-protein when expressed in a host or host cell, when compared to the yield of a corresponding S-protein without the one or more than one substitutions expressed in the host or host cell.

The one or more than one amino acid substitution may correspond to amino acids at positions 667, 668, 670, 802, 923, 927, 971, 972 or a combination thereof, when compared to reference amino acid sequence of SEQ ID NO: 2.

In one aspect, the one or more than one amino acid substitution correspond to amino acids at positions 971 and 972, when compared to reference amino acid sequence of SEQ ID NO: 2. In another aspect, the one or more than one amino acid substitution correspond to amino acids at positions 802, 927, 971 and 972, when compared to reference amino acid sequence of SEQ ID NO: 2. Furthermore, the modified S-protein may comprise one or more than one amino acid substitution corresponding to amino acids at positions 667, 668, 670, or a combination thereof, when compared to reference amino acid sequence of SEQ ID NO: 2. Accordingly, the modified S-protein may comprise substitutions that correspond to amino acids at positions 667, 668 and 670, when compared to reference amino acid sequence of SEQ ID NO: 2.

In one aspect, the one or more than one substitution may correspond to amino acids at positions 667, 668, 670, 971 and 972, when compared to reference amino acid sequence of SEQ ID NO: 2. The substitution of the amino acid corresponding to the amino acid at position 667 of SEQ ID NO: 2 may be to glycine or a conserved substitution of glycine, the substitution of the amino acid corresponding to position 668 of SEQ ID NO: 2 may be to a serine or a conserved substitution of serine, the substitution of the amino acid corresponding to position 670 of SEQ ID NO: 2 may be to a serine or a conserved substitution of serine, the substitution of the amino acid corresponding to the amino acid at position 971 of SEQ ID NO: 2 may be to a proline or a conserved substitution of proline and the substitution of the amino acid corresponding to the amino acid at position 972 of SEQ ID NO: 2 may be to a proline or a conserved substitution of proline. The modified S-protein as described above may further comprises an amino acid substitution corresponding to amino acid at position 923, when compared to reference amino acid sequence of SEQ ID NO: 2.

In another aspect the one or more than one amino acid substitution may correspond to amino acids at positions 667, 668, 670, 802, 927, 971 and 972, when compared to reference amino acid sequence of SEQ ID NO: 2. The substitution of the amino acid corresponding to the amino acid at position 667 of SEQ ID NO: 2 may be to glycine or a conserved substitution of glycine, the substitution of the amino acid corresponding to position 668 of SEQ ID NO: 2 may be to a serine or a conserved substitution of serine, the substitution of the amino acid corresponding to position 670 of SEQ ID NO: 2 may be to a serine or a conserved substitution of serine, the substitution of the amino acid corresponding to the amino acid at positions 802 of SEQ ID NO: 2 may be to a proline or a conserved substitution of proline, the substitution of the amino acid corresponding to the amino acid at positions 927 of SEQ ID NO: 2 may be to a proline or a conserved substitution of proline, the substitution of the amino acid corresponding to the amino acid at positions 971 of SEQ ID NO: 2 may be to a proline or a conserved substitution of proline and the substitution of the amino acid corresponding to the amino acid at positions 972 of SEQ ID NO: 2 may be to a proline or a conserved substitution of proline.

In another aspect the modified S-protein as described above may further comprises an amino acid substitution corresponding to amino acid at position 923, when compared to reference amino acid sequence of SEQ ID NO: 2. The substitution in the modified S-protein of the amino acid corresponding to the amino acid at position 667 of SEQ ID NO: 2 may be to glycine or a conserved substitution of glycine, the substitution of the amino acid corresponding to position 668 of SEQ ID NO: 2 may be to a serine or a conserved substitution of serine, the substitution of the amino acid corresponding to position 670 of SEQ ID NO: 2 may be to a serine or a conserved substitution of serine, the substitution of the amino acid corresponding to the amino acid at positions 802, 927, 971 and 972 of SEQ ID NO: 2 may be to a proline or a conserved substitution of proline and the substitution of the amino acid corresponding to position 923 of SEQ ID NO: 2 may be to phenylalanine or a conserved substitution of phenylalanine.

The modified the S-protein may comprise from 80% to 100% identity with amino acids of SEQ ID NO: 5, 21, 30, 47, 48, 49, 50, 51, 52, 53, 54, 55, 56, 57, 58, 59, 60, 61, 62, 95, 96, 97, 108, 109, 110, 144, 145, 146, 155, 156 or 157, or with amino acids 24-1259 of SEQ ID NO: 47 amino acids 25-1259 of SEQ ID NO: 48, amino acids 25-1259 of SEQ ID NO: 49, amino acids 25-1259 of SEQ ID NO: 50, amino acids 25-1259 of SEQ ID NO: 51, amino acids 25-1259 of SEQ ID NO: 52, amino acids 25-1259 of SEQ ID NO: 53, amino acids 25-1259 of SEQ ID NO: 54, amino acids 25-1259 of SEQ ID NO: 55, amino acids 25-1259 of SEQ ID NO: 56, amino acids 25-1259 of SEQ ID NO: 57, amino acids 25-1259 of SEQ ID NO: 58, amino acids 25-1262 of SEQ ID NO: 59, amino acids 25-1261 of SEQ ID NO: 60, amino acids 25-1258 of SEQ ID NO: 61, or amino acids 25-1256 of SEQ ID NO: 62, amino acids 25-1243 of SEQ ID NO: 95, amino acids 25-1240 of SEQ ID NO: 96, amino acids 25-1243 of SEQ ID NO: 97, amino acids 25-1341 of SEQ ID NO: 108, amino acids 25-1338 of SEQ ID NO: 109, amino acids 25-1341 of SEQ ID NO: 110, amino acids 25-1351 of SEQ ID NO: 144, amino acids 25-1348 of SEQ ID NO: 145, amino acids 25-1351 of SEQ ID NO: 146, amino acids 25-1159 of SEQ ID NO: 155, amino acids 25-1156 of SEQ ID NO: 156, or amino acids 25-1159 of SEQ ID NO: 157.

In another aspect, it is provided a nucleic acid that comprises a nucleotide sequence that encodes the modified S-protein as described above.

In a further aspect a composition comprising an effective dose of the modified S-protein, the trimer comprising the modified S-protein or VLP comprising the modified S-protein as described above and a pharmaceutically acceptable carrier, adjuvant, vehicle or excipient is provided. In yet another aspect, it is provided a vaccine for inducing an immune response. The vaccine comprises an effective dose of the modified S protein, the trimer comprising the modified S-protein or VLP comprising the modified S-protein as described above as described above.

The composition further comprises a pharmaceutically acceptable carrier, adjuvant, vehicle or excipient. In a further aspect, it is provided a vaccine for inducing an immune response. The vaccine comprises an effective dose of the VLP comprising a modified coronavirus as described above. The vaccine may be a multivalent vaccine, comprising a mixture of VLP.

In yet another aspect a (non-human) host or host cell comprising the modified S-protein, trimer or VLP as described above is provided. In yet another aspect a host or host cell comprising the VLP as described above is provided. In another aspect it is provided a composition comprising an effective dose of the VLP comprising the modified S-protein as described above is provided.

In yet another aspect, a S-protein trimer is provided. The trimer comprises modified coronavirus S-protein, the modified S-protein comprising
an ectodomain derived from a coronavirus S-protein,
a transmembrane and cytosolic tail domain (TMCT), wherein the TMCT is a chimeric TMCT, comprising:
a transmembrane domain (TM), wherein the TM or a portion of the TM is derived from a coronavirus S-protein, and
a cytosolic tail (CT), wherein the CT or a portion of the CT is derived from an influenza hemagglutinin (HA) protein. The modified S-protein in the trimer may comprise one or more than one amino acid substitution when compared to a wild-type coronavirus amino acid sequence, as described above. In a further aspect a composition comprising an effective dose of the trimer as described above and a pharmaceutically acceptable carrier, adjuvant, vehicle or excipient is provided. In another aspect, virus like particle (VLP) comprising the trimer as described above are also provided. The VLP may further comprise plant lipids. In another aspect it is provided a composition comprising an effective dose of the VLP comprising the trimer as described above and a pharmaceutically acceptable carrier, adjuvant, vehicle or excipient is provided. In a further aspect, it is provided a vaccine for inducing an immune response. The vaccine comprises an effective dose of the trimer as described above. In a further aspect, it is provided a vaccine for inducing an immune response. The vaccine comprises an effective dose of the VLP comprising the trimer as described above. The vaccine may be a multivalent vaccine, comprising a mixture of VLP. In yet another aspect a non-human host or host cell comprising the trimer or the VLP comprising the trimer as described above is provided.

In another aspect, it is provided a method for inducing immunity to a Coronavirus infection in a subject, the method comprising administering the composition or vaccines as described above. The composition or vaccine may be administered once to the subject or the composition or vaccine may be administered multiple times to the subject. The composition or vaccine may be administered as an initial dose and one or more than one subsequent doses may be administered between 1 day and 6 month from the administration of the initial dose. The subsequent dose may be administered after 21 days from the administration of the initial dose.

In another aspect an antibody or antibody fragment prepared using the composition or vaccine as described above are provided.

In yet another aspect, it is provided A) a method of producing a virus like particle (VLP) in a (non-human) host or host cell comprising:
a) introducing into a non-human host or host cell the nucleic acid comprising a nucleotide sequence that encodes the modified S-protein as described above; or providing the non-human host or host cell comprising the nucleic acid comprising a nucleotide sequence that encodes the modified S-protein as described above, and
b) incubating the non-human host or host cell under conditions that permit the expression of the nucleic acid, thereby producing the VLP.

In a further step c) the non-human host or host cell may be harvested.

In a further aspect, it is provided B) a method of increasing yield of production of a Coronavirus S-protein in a (non-human) host or host cell comprising:
a) introducing the nucleic acid comprising a nucleotide sequence that encodes the modified S-protein as described above into the non-human host or host cell; or providing a non-human host or host cell comprising the nucleic acid comprising a nucleotide sequence that encodes the modified S-protein as described above; and
b) incubating the non-human host or host cell under conditions that permit expression of the modified S-protein encoded by the nucleic acid, thereby producing modified S-protein at a higher yield compared to a host or host cell expressing the unmodified S-protein under similar or identical conditions.

In a further step c) the non-human host or host cell may be harvested.

In yet another aspect, it is provided C) a method of increasing yield of production of virus like particle (VLP) in a (non-human) host or host cell comprising:
a) introducing into the non-human host or host cell, a nucleic acid encoding a modified coronavirus S-protein comprising
   an ectodomain derived from a coronavirus S-protein,
   a transmembrane and cytosolic tail domain (TMCT), wherein the TMCT is a chimeric TMCT, comprising:
      a transmembrane domain (TM), wherein the TM or a portion of the TM is derived from a coronavirus S-protein and
      a cytosolic tail (CT), wherein the CT or a portion of the CT is derived from an influenza hemagglutinin (HA) protein;
providing a non-human host or host cell comprising the nucleic acid encoding the modified S-protein comprising
   an ectodomain derived from a coronavirus S-protein,
   a transmembrane and cytosolic tail domain (TMCT), wherein the TMCT is a chimeric TMCT, comprising:
      a transmembrane domain (TM), wherein the TM or a portion of the TM is derived from a coronavirus S-protein and
      a cytosolic tail (CT), wherein the CT or a portion of the CT is derived from an influenza hemagglutinin (HA) protein; and
b) incubating the non-human host or host cell under conditions that permit expression of the modified S-protein encoded by the nucleic acid, thereby producing VLP comprising modified S-protein at a higher yield compared to the yield of VLP in a host of host cell that expresses unmodified S protein under similar or identical conditions.

In a further aspect, it is provided D) a method of producing a virus like particle (VLP) in a (non-human) host or host cell comprising:
a) introducing into a non-human host or host cell, a nucleic acid encoding a modified coronavirus S-protein comprising
   an ectodomain derived from a coronavirus S-protein,
   a transmembrane and cytosolic tail domain (TMCT), wherein the TMCT is a chimeric TMCT, comprising:
      a transmembrane domain (TM), wherein the TM or a portion of the TM is derived from a coronavirus S-protein and
      a cytosolic tail (CT), wherein the CT or a portion of the CT is derived from an influenza hemagglutinin (HA) protein; or
providing a non-human host or host cell comprising the nucleic acid encoding the modified S-protein comprising
   an ectodomain derived from a coronavirus S-protein,
   a transmembrane and cytosolic tail domain (TMCT), wherein the TMCT is a chimeric TMCT, comprising:
      a transmembrane domain (TM), wherein the TM or a portion of the TM is derived from a coronavirus S-protein and
      a cytosolic tail (CT), wherein the CT or a portion of the CT is derived from an influenza hemagglutinin (HA) protein; and
b) incubating the non-human host or host cell under conditions that permit the expression of the nucleic acid, thereby producing the VLP.

In a further aspect the VLP of method A), B), C) or D) may further be extracted and purified from the host or host cell. The host or host cell may comprise a plant, a plant cell, a fungi, a fungi cell, an insect, an insect cell, an animal or an animal cell. The host or host cell of method A), B), C) or D) may be a plant, portion of a plant or plant cell.

In another aspect it is provided a VLP produced by the method of A), B), C) or D).

Furthermore, in yet another aspect it is provided a composition comprising an adjuvant and virus-like particles (VLP), the VLP comprising modified coronavirus S-protein, the modified S-protein comprising
an ectodomain derived from a coronavirus S-protein,
a transmembrane and cytosolic tail domain (TMCT), wherein the TMCT is a chimeric TMCT, comprising:
   a transmembrane domain (TM), wherein the TM or a portion of the TM is derived from a coronavirus S-protein and
   a cytosolic tail (CT), wherein the CT or a portion of the CT is derived from an influenza hemagglutinin (HA) protein and wherein the modified S-protein further comprises substitutions at positions 667, 668, 670, 971 and 972 when compared to reference amino acid sequence of SEQ ID NO: 2.

In yet another aspect it is provided a composition comprising virus-like particle (VLP), the VLP comprising modified coronavirus S-protein, the modified S protein comprising
an ectodomain derived from a coronavirus S-protein,
a transmembrane and cytosolic tail domain (TMCT), wherein the TMCT is a chimeric TMCT, comprising:
   a transmembrane domain (TM), wherein the TM or a portion of the TM is derived from a coronavirus S-protein and
   a cytosolic tail (CT), wherein the CT or a portion of the CT is derived from an influenza hemagglutinin (HA) protein; and wherein the modified S-protein comprises a glycine substitution at position 667, a serine substitution at position 668, a serine substitution at position 670, a proline substitution at position 971 and a proline substitution at position 972, the position corresponding to reference amino acid sequence of SEQ ID NO: 2. The influenza hemagglutinin (HA) protein may be derived from influenza type B or influenza subtype H1, H3, H5, H6, H7 or H9. The composition may further comprise an adjuvant.

In a further aspect it is provided a composition comprising virus-like particle (VLP), the VLP comprising modified coronavirus S-protein, the modified S protein comprising the sequence of SEQ ID NO: 21. The composition may further comprise an adjuvant.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows a schematic representation of Coronavirus S protein and the location of S1/S2 (aa 685/686) and S2′ cleavage sites. SP: signal peptide (aa 1-15); NTD: N-terminal domain (aa 16-306); RBD: receptor-binding domain (aa 335-527); FP: fusion peptide (aa 816-833); HR1: heptad repeat 1 (aa 908-991); HR2: heptad repeat 2 (aa 1166-1207); TM: transmembrane domain (aa 1214-1234); CT: cytoplasmic tail (aa 1235-1273). The residue numbers (aa) of each region correspond to their positions in the S protein of SARS-CoV-2 (2019-nCoV).

FIG. 2 shows an alignment of amino acid sequences from exemplary influenza strains. The C-terminal region of the ectodomain, the transmembrane domain TM, and the cytoplasmic tail domain (CT) of hemagglutinin (HA) are shown for H1 A/California/07/2009 (SEQ ID NO: 6), H2 A/Singapore/1/1957 HA (SEQ ID NO:7), H3 A/Minnesota/41/2019 HA (SEQ ID NO:8), H5 A/Indonesia/5/05 HA (SEQ ID NO:9), H6 A/Teal/Hong Kong/W312/97 HA (SEQ ID NO:10), H7 A/Guangdong/17SF003/2016 HA (SEQ ID NO:11), H9 A/Hong Kong/1073/99 HA (SEQ ID NO:12), and B/Washington/02/2019 HA (SEQ ID NO:13). The consensus sequence for these sequences is also shown (SEQ ID NO: 14).

FIG. 3A shows quantified fold-change difference in SARS-CoV-2 S protein accumulation in plants expressing: SARS-CoV-2 S protein with a native (wild-type) transmembrane domain and cytoplasmic tail (wtTMCT) under the control of the following 5′UTRs: nbMT78 (construct 8586), nbCSY65 (construct 8589) and nbHEL40 (construct 8591); a modified SARS-CoV-2 protein wherein the native (wild-type) transmembrane domain and cytoplasmic tail (wtTMCT) has been replaced with the TMCT of influenza hemagglutinin (HA) of influenza H5 A/Indonesia/5/05 (H5iTMCT) under the control of nbMT78 (construct 8592), nbCSY65 (construct 8595) and nbHEL40 (construct 8597); and a modified SARS-CoV-2 protein wherein the native (wild-type) cytoplasmic tail (wtCT) has been replaced with the CT of influenza hemagglutinin (HA) of influenza H5 A/Indonesia/5/05 (H5iCT) under the control of nbMT78 (construct 8610), nbCSY65 (construct 8611) and nbHEL40 (construct 8671). The SARS-CoV-2 S protein sequences (referred to as nCOV S (GSAS-2P)) have the following substitutions: R667G, R668S, R670S, K971P and V972P with respect to the reference sequence of SEQ ID NO: 2. The results have been normalized to the SARS-CoV-2 S protein accumulation from construct 8591, which is set as 1. FIG. 3B shows protein separation of clarified crude extract on a non-reducing SDS-PAGE gel. The following modified S proteins were expressed in plants: Lane 1: S protein with wild-type transmembrane and cytosolic tail domain (wt TMCT) under the control of nbMT78; lane 2: S protein with wild-type transmembrane and cytosolic tail domain (wt TMCT) under the control of nbCSY65; lane 3: S protein with wild-type transmembrane and cytosolic tail domain (wt TMCT) under the control of nbHEL40; lane 4: modified S protein with a SARS-CoV-2 ectodomain, and a transmembrane and cytosolic tail domain from hemagglutinin (HA) of H5 A/Indonesia/5/05 (H5i TMCT) under the control of nbMT78; lane 5: modified S protein with a SARS-CoV-2 ectodomain and a transmembrane and cytosolic tail domain from hemagglutinin (HA) of H5 A/Indonesia/5/05 (H5i TMCT) under the control of nbCSY65; lane 6: modified S protein with a SARS-CoV-2 ectodomain and a transmembrane and cytosolic tail domain from hemagglutinin (HA) of H5 A/Indonesia/5/05 (H5i TMCT) under the control of nbHEL40; lane 7: modified S protein with a SARS-CoV-2 ectodomain, a SARS-CoV-2 transmembrane domain and cytosolic tail domain (CT) from hemagglutinin (HA) of influenza H5 A/Indonesia/5/05 (H5i CT) under the control of nbMT78; lane 8: modified S protein with a SARS-CoV-2 ectodomain, a SARS-CoV-2 transmembrane domain and cytosolic tail domain (CT) from hemagglutinin (HA) of influenza H5 A/Indonesia/5/05 (H5i CT) under the control of nbCSY65; lane 9: modified S protein with a SARS-CoV-2 ectodomain, a SARS-CoV-2 transmembrane domain and cytosolic tail domain (CT) from hemagglutinin (HA) of influenza H5 A/Indonesia/5/05 (H5i CT) under the control of nbHEL40. The modified S protein has a molecular weight of about 150 kDa and is indicated by an arrow. FIG. 3C shows a Western blot analysis of the same series of lysates depicted in FIG. 3B and the lanes correspond to the lanes as described in FIG. 3B. The top panel shows detection with an anti-SARS-CoV-2 S1 antibody (40150-R007). The bottom panel shows detection with an anti-SARS-CoV-2 S2 antibody (NB100-56578). The monomer of the SARS-CoV-2 protein (comprising the S1 and S2 subunit) has a molecular weight of about 150 kDa (non-reducing).

FIG. 4A shows quantified fold-change difference in SARS-CoV-2 S protein accumulation in plants expressing: a modified S protein with a SARS-CoV-2 ectodomain, a SARS-CoV-2 transmembrane, and cytosolic tail domain from hemagglutinin (HA) of H5 A/Indonesia/5/05 (H5 Indo); a modified S protein with a SARS-CoV-2 ectodomain, a SARS-CoV-2 transmembrane, and cytosolic tail domain from hemagglutinin (HA) of H1 A/California/7/2009 (H1 California); a modified S protein with a SARS-CoV-2 ectodomain, a SARS-CoV-2 transmembrane, and cytosolic tail domain from hemagglutinin (HA) of H3 A/Minnesota/41/2019 (H3 Minnesota); a modified S protein with a SARS-CoV-2 ectodomain, a SARS-CoV-2 transmembrane, and cytosolic tail domain from hemagglutinin (HA) of H6 A/Teal/Hong Kong/W312/97 (H6 Hong Kong); a modified S protein with a SARS-CoV-2 ectodomain, a SARS-CoV-2 transmembrane, and cytosolic tail domain from hemagglutinin (HA) of H7 A/Guangdong/17SF003/2016 (H7 Guangdong); a modified S protein with a SARS-CoV-2 ectodomain, a SARS-CoV-2 transmembrane, and cytosolic tail domain from hemagglutinin (HA) of H9 A/Hong Kong/1073/99 (H9 Hong Kong); and a modified S protein with a SARS-CoV-2 ectodomain, a SARS-CoV-2 transmembrane, and cytosolic tail domain from hemagglutinin (HA) of B/Washington/02/2019 (B Washington). The results have been normalized to the SARS-CoV-2 S protein accumulation from construct 8671 encoding a modified SARS-CoV-2 protein wherein the native (wild-type) cytoplasmic tail (wtCT) has been replaced with the CT of influenza hemagglutinin (HA) of influenza H5 A/Indonesia/5/05 (H5iCT) under the control of nbHEL40 (H5 Indo), which is set as 1.

FIG. 4B shows a Western blot analysis of crude lysate from plants expressing the modified S proteins from the following constructs: lane 2, a modified S protein with a SARS-CoV-2 ectodomain, a SARS-CoV-2 transmembrane, and cytosolic tail domain from hemagglutinin (HA) of H5 A/Indonesia/5/05 (H5 Indo); lane 3, a modified S protein with a SARS-CoV-2 ectodomain, a SARS-CoV-2 transmembrane, and cytosolic tail domain from hemagglutinin (HA) of H1 A/California/07/2009 (H1 Calif); lane 4, a modified S protein with a SARS-CoV-2 ectodomain, a SARS-CoV-2 transmembrane, and cytosolic tail domain from hemagglutinin (HA) of H3 A/Minnesota/41/2019 (H3 Minn); lane 5, a modified S protein with a SARS-CoV-2 ectodomain, a SARS-CoV-2 transmembrane, and cytosolic tail domain from hemagglutinin (HA) of H6 A/Teal/Hong Kong/W312/97 (H6 HK); lane 6, a modified S protein with a SARS-CoV-2 ectodomain, a SARS-CoV-2 transmembrane, and cytosolic tail domain from hemagglutinin (HA) of H7 A/Guangdong/17SF003/2016 (H7 Guan); lane 7, a modified S protein with a SARS-CoV-2 ectodomain, a SARS-CoV-2 transmembrane, and cytosolic tail domain from hemagglutinin (HA) of H9 A/Hong Kong/1073/99 (H9 HK); lane 8, a modified S protein with a SARS-CoV-2 ectodomain, a SARS-CoV-2 transmembrane, and cytosolic tail domain from hemagglutinin (HA) of B/Washington/02/2019 (B Wash). Lane 1 is crude lysate from a plant treated with a mock Agroinfiltration. Sino 400150-R007 antibody was used for detection of the S1 subunit of the SARS-COV-2 S protein. The monomer of the SARS-CoV-2 protein (comprising the S1 and S2 subunit) has a molecular weight of about 150 kDa (non-reducing). FIG. 4C shows a Western blot analysis of the same series of lysates depicted in FIG. 4B, except with NB100-56578 antibody detecting the S2 subunit of the SARS-COV-2 S protein.

FIG. 5A shows the amino acid sequence of the C-terminal region of the native SARS-CoV-2 S protein (wtTM/wtCT), the C-terminal region of influenza H5 hemagglutinin (HA) (H5iTM/H5iCT), the C-terminal region of modified SARS-CoV-2 S protein with wild-type transmembrane domain (TM) and influenza H5 HA cytosolic tail (CT) domain (wtTM/H5iCT), and the C-terminal regions of four alternative versions of modified S protein (wtTM/H5iCT V1-V4) with variable margin between the SARS-CoV-2 transmembrane (TM) domain and H5 A/Indonesia/5/05 HA cytosolic tail (CT) domain. The TM domain from Coronavirus S-protein is underlined and the CT domain derived from influenza HA is shown in bold. FIG. 5B shows quantified fold-change difference in SARS-CoV-2 S protein accumulation in plants expressing each of the four variant modified S proteins with a chimeric transmembrane and cytosolic tail domain (TMCT), as depicted in FIG. 5A (wtTM/H5iCT, V1-V4), relative to modified SARS-CoV-2 S protein accumulation in plants expressing modified SARS-CoV-2 S protein having a chimeric TMCT with a wild-type transmembrane domain (TM) and influenza H5 HA cytosolic tail (CT) domain (wtTM/H5iCT) which is set as 1.

FIG. 6A shows an electron micrograph of virus like particles (VLP) comprising SARS-COV-2 S protein with wild-type transmembrane and cytosolic tail domain (wtTMCT; construct 8591) FIG. 6B shows an electron micrograph of virus like particles (VLP) comprising a modified S protein with a SARS-CoV-2 ectodomain and an influenza H5 hemagglutinin transmembrane domain and cytosolic tail domain (H5i TMCT; construct 8597). FIG. 6C shows an electron micrograph of virus like particles (VLP) comprising a modified S protein with a SARS-CoV-2 ectodomain, a SARS-CoV-2 transmembrane domain and an influenza H5 hemagglutinin cytosolic tail domain (H5i CT; construct 8671). FIG. 6D shows an electron micrograph of virus like particles (VLP) comprising an alternative version of modified S protein (H5i CT V1; construct 8980) having a SARS-CoV-2 ectodomain and a chimeric transmembrane and cytosolic tail domain (TMCT). FIG. 6E shows an electron micrograph of virus like particles (VLP) comprising an alternative version of modified S protein (H5i CT V2; construct 8981) having a SARS-CoV-2 ectodomain and a chimeric transmembrane and cytosolic tail domain (TMCT). FIG. 6F shows an electron micrograph of virus like particles (VLP) comprising an alternative version of modified S protein (H5i CT V3; construct 8982) with a SARS-CoV-2 ectodomain having a SARS-CoV-2 ectodomain and a chimeric transmembrane and cytosolic tail domain (TMCT). FIG. 6G shows an electron micrograph of virus like particles (VLP) comprising an alternative version of modified S protein (H5i CT V4; construct 8983) having a SARS-CoV-2 ectodomain and a chimeric transmembrane and cytosolic tail domain (TMCT). FIG. 6H shows an electron micrograph of virus like particles (VLP) comprising a modified S protein with a SARS-CoV-2 ectodomain, a SARS-CoV-2 transmembrane domain and an influenza H1 hemagglutinin cytosolic tail domain (H1 CT; construct 7390). FIG. 6I shows an electron micrograph of virus like particles (VLP) comprising a modified S protein with a SARS-CoV-2 ectodomain, a SARS-CoV-2 transmembrane domain and an influenza H3 hemagglutinin cytosolic tail domain (H3 CT; construct 7391). FIG. 6J shows an electron micrograph of virus like particles (VLP) comprising a modified S protein with a SARS-CoV-2 ectodomain, a SARS-CoV-2 transmembrane domain and an influenza H6 hemagglutinin cytosolic tail domain (H6 CT; construct 7392). FIG. 6K shows an electron micrograph of virus like particles (VLP) comprising a modified S protein with a SARS-CoV-2 ectodomain, a SARS-CoV-2 transmembrane domain and an influenza H7 hemagglutinin cytosolic tail domain (H7 CT; construct 7393). FIG. 6L shows an electron micrograph of virus like particles (VLP) comprising a modified S protein with a SARS-CoV-2 ectodomain, a SARS-CoV-2 transmembrane domain and an influenza H9 hemagglutinin cytosolic tail domain (H9 CT; construct 7394). FIG. 6M shows an electron micrograph of virus like particles (VLP) comprising a modified S protein with a SARS-CoV-2 ectodomain, a SARS-CoV-2 transmembrane domain and an influenza HA B hemagglutinin cytosolic tail domain (HA B CT; construct 7395).

FIG. 7A shows a schematic representation of acceptor vector 8501. FIG. 7B shows a schematic representation of acceptor vector 8500. FIG. 7C shows a schematic representation of acceptor vector 8716.

FIG. 8A shows a schematic representation of vector 8586. FIG. 8B shows a schematic representation of vector 8589. FIG. 8C shows a schematic representation of vector 8591.

FIG. 9A shows a schematic representation of vector 8592. FIG. 9B shows a schematic representation of vector 8595. FIG. 9C shows a schematic representation of vector 8597.

FIG. 10A shows a schematic representation of vector 8610. FIG. 10B shows a schematic representation of vector 8611. FIG. 10C shows a schematic representation of vector 8671.

FIG. 11A shows quantified fold-change of accumulation in plants expressing modified SARS-CoV-2 S protein (wtTM/H5iCT) with additional substitutions. The modified SARS-CoV-2 S proteins have the following substitutions: “GSAS-2P”: R667G, R668S, R670S, K971P and V972P; “GSAS-4P”: R667G, R668S, R670S, K971P, V972P, F802P and A927P; and “GSAS-6P”: R667G, R668S, R670S, K971P, V972P, F802P, A877P, A884P and A927P (with respect to reference sequence of SEQ ID NO: 2). The results have been normalized to the accumulation of modified SARS-CoV-2 (wtTM/H5iCT) and GSAS+2P substitutions, which is set as 1. FIG. 11B shows quantified fold-change of accumulation in plants expressing modified SARS-CoV-2 S protein (wtTM/H5iCT) with each of the GSAS-2P, GSAS-4P, and GSAS-6P substitutions as described for FIG. 11A as compared to the quantified fold change of accumulation wherein each modified SARS-CoV-2 S protein further incorporates a L923F substitution. The results have been normalized to the accumulation of modified SARS-CoV-2 (wtTM/H5iCT) and GSAS+2P substitutions, which is set as 1.

FIG. 12A shows a schematic representation of vector 8980. FIG. 12B shows a schematic representation of vector 8981. FIG. 12C shows a schematic representation of vector 8982. FIG. 12D shows a schematic representation of vector 8983.

FIG. 13A shows a schematic representation of vector 7390. FIG. 13B shows a schematic representation of vector 7391. FIG. 13C shows a schematic representation of vector 7392. FIG. 13D shows a schematic representation of vector 7393. FIG. 13E shows a schematic representation of vector 7394. FIG. 13F shows a schematic representation of vector 7395.

FIG. 14A shows a schematic representation of vector 8953. FIG. 14B shows a schematic representation of vector 8940.

FIG. 15A shows a schematic representation of vector 8933. FIG. 15B shows a schematic representation of vector 8960. FIG. 15C shows a schematic representation of vector 8947.

FIG. 16A shows a Western blot analysis of crude lysate from plants expressing the modified S proteins from the following constructs: lane 1, a modified S protein with a SARS-CoV-1 ectodomain, transmembrane, and cytosolic tail domain (“wtTMCT”, construct 9231); lane 2, a modified S protein with an ectodomain from SARS-CoV-1, and a transmembrane and cytosolic tail domain (TMCT) from hemagglutinin (HA) of H5 A/Indonesia/5/05 (“H5iTMCT”, construct 9232); lane 3, a modified S protein with an ectodomain and transmembrane domain from SARS-CoV-1 and a cytosolic tail domain from hemagglutinin (HA) of H5 A/Indonesia/5/05 (H5 Indo) (“H5iCT”, construct 9233); lane 4, a modified S protein with an ectodomain and transmembrane domain from SARS-CoV-1 and a cytosolic tail domain from hemagglutinin (HA) of H5 A/Indonesia/5/05 (H5 Indo) with a variable margin between the SARS-CoV-1 transmembrane (TM) domain and H5 A/Indonesia/5/05 HA cytosolic tail (CT) domain (“H5iCT(V4)”, construct 9234); lane 5, a modified S protein with an ectodomain and transmembrane domain from a SARS-CoV-1 and a cytosolic tail domain from hemagglutinin (HA) of H1 A/California/7/2009 (“H1cCT”, construct 9235). The primary antibody used for detection was SARS-CoV Spike S1 Subunit Antibody from Sino Biologicals (40150-MM08, 1/5000). The secondary antibody used for detection was Goat anti-Mouse from JIR (115-035-146, 1/10000). The modified S protein has a molecular weight of about 150 kDa.

FIGS. 16B, 16C and 16D shows Western blot analysis of fractions F5 (30%), F6 (30%), F7 (25%), F8 (25%), F9 (25%), F10 (15%) and F11 (15%) from a discontinuous iodixanol density gradient. Accumulation of protein in these fractions is indicative for the formation of higher molecular weight structures i.e. VLP formation. For Western blots from fractions of crude lysate, the primary antibody used for detection was SARS-CoV Spike S1 subunit antibody from Sino Biologicals, 40150-MM08 (1/5000) and the secondary antibody used for detection was Goat anti-Mouse, JIR, 115-035-146 (1/10000). FIG. 16B: Crude lysate from plants expressing SARS-CoV-1 S protein (with 2P+R667A substitution), with a native TMCT domain (wtTMCT, construct 9231) were analyzed. FIG. 16C: Crude lysate from plants expressing modified SARS-CoV-1 S protein (with 2P+R667A substitution) having a TMCT from H5 A/Indonesia/5/05 HA (H5iTMCT, construct 9232). FIG. 16D: Crude lysate from plants expressing modified SARS-CoV-1 S protein (with 2P+R667A substitution) having a cytoplasmic tail from H5 A/Indonesia/5/05 HA (H5iCT, construct 9233).

FIG. 17A shows an electron micrograph of virus like particles (VLP) comprising SARS-COV-1 S protein (with 2P+R667A substitution) with native TMCT domain (wtTMCT, construct 9231). FIG. 17B shows an electron micrograph of virus like particles (VLP) comprising modified SARS-CoV-1 S protein (with 2P+R667A substitution) having a TMCT from H5 A/Indonesia/5/05 HA (H5iTMCT, construct 9232). FIG. 17C shows an electron micrograph of virus like particles (VLP) comprising modified SARS-CoV-1 S protein (with 2P+R667A substitution) having a cytoplasmic tail from H5 A/Indonesia/5/05 HA (H5iCT, construct 9233).

FIG. 18A shows a schematic representation of vector 9231. FIG. 18B shows a schematic representation of vector 9232. FIG. 18C shows a schematic representation of vector 9233. FIG. 18D shows a schematic representation of vector 9234. FIG. 18E shows a schematic representation of vector 9235.

FIG. 19A shows a Western blot analysis of crude lysate from plants expressing the modified S proteins from the following constructs: lane 1, a modified S protein with a MERS-CoV ectodomain, transmembrane, and cytosolic tail domain (“wtTMCT”, construct 9246); lane 2, a modified S protein with an ectodomain from MERS-CoV, and a transmembrane and cytosolic tail domain (TMCT) from hemagglutinin (HA) of H5 A/Indonesia/5/05 (“H5iTMCT”, construct 9247); lane 3, a modified S protein with an ectodomain and transmembrane domain from MERS-CoV and a cytosolic tail domain from hemagglutinin (HA) of H5 A/Indonesia/5/05 (H5 Indo) (“H5iCT”, construct 9249); lane 4, a modified S protein with an ectodomain and transmembrane domain from MERS-CoV and a cytosolic tail domain from hemagglutinin (HA) of H5 A/Indonesia/5/05 (H5 Indo) with a variable margin between the MERS-CoV transmembrane (TM) domain and H5 A/Indonesia/5/05 HA cytosolic tail (CT) domain (“H5iCT(V4)”, construct 9250); lane 5, a modified S protein with an ectodomain and transmembrane domain from a MERS-CoV and a cytosolic tail domain from hemagglutinin (HA) of H1 A/California/7/2009 (“H1cCT”, construct 9251). The primary antibody used for detection was MERS-CoV spike protein S1 antibody (N-terminal) from Sino Biological, (100208-RP02, 1/5000). The secondary antibody used for detection was Goat anti-Mouse from JIR (115-035-144, 1/10000). The modified S protein has a molecular weight of about 175 kDa. FIG. 19B shows an electron micrograph of virus like particles (VLP) comprising MERS-COV S protein (with ASVG+2P substitution) with native TMCT domain (wtTMCT, construct 9246). FIG. 19C shows an electron micrograph of virus like particles (VLP) comprising modified MERS-CoV S protein (with ASVG+2P substitution) having a TMCT from H5 A/Indonesia/5/05 HA (H5iTMCT, construct 9247). FIG. 19D shows an electron micrograph of virus like particles (VLP) comprising modified MERS-CoV S protein (with ASVG+2P substitution) having a cytoplasmic tail from H5 A/Indonesia/5/05 HA (H5iCT, construct 9249). FIG. 19E shows an electron micrograph of virus like particles (VLP) comprising modified MERS-CoV S protein (with ASVG+2P substitution) having a cytoplasmic tail from H5 A/Indonesia/5/05 HA with a variable margin between the MERS-CoV transmembrane (TM) domain and H5 A/Indonesia/5/05 HA cytosolic tail (CT) domain (H5iCT (V4), construct 9250). FIG. 19F shows an electron micrograph of virus like particles (VLP) comprising modified MERS-CoV S protein (with ASVG+2P substitution) having a cytoplasmic tail from H1 A/California/7/2009 HA (H1cCT, construct 9251).

FIG. 20A shows a schematic representation of vector 9246. FIG. 20B shows a schematic representation of vector 9247. FIG. 20C shows a schematic representation of vector 9249. FIG. 20D shows a schematic representation of vector 9250. FIG. 20E shows a schematic representation of vector 9251.

FIG. 21 shows a schematic representation of acceptor vector 7147.

FIG. 22 shows an alignment of the native SARS-CoV-2, SARS-CoV-1, and MERS-CoV S protein sequences with the native signal peptide removed (SEQ ID NO: 2, 114, and 115). Residues corresponding to the RRAR furin cleavage site (667-670) and each of F802P, A877P, A884P, A927P, K971P, V972P in native SARS-CoV-2 S protein without signal peptide (SEQ ID NO: 2) are boxed along with homologous residues from native SARS-CoV-1 S protein without signal peptide (SEQ ID NO: 114) and native MERS S protein (SEQ ID NO: 115).

FIG. 23A shows a Western blot analysis of crude lysate from plants expressing the modified S proteins from the following constructs: lane 3, a modified S protein with a OC43-CoV ectodomain, transmembrane, and cytosolic tail domain (“wtTMCT”, construct 9269); lane 4, a modified S protein with an ectodomain from OC43-CoV, and a transmembrane and cytosolic tail domain (TMCT) from hemagglutinin (HA) of H5 A/Indonesia/5/05 (“H5iTMCT”, construct 9270); lane 5, a modified S protein with an ectodomain and transmembrane domain from OC43-CoV and a cytosolic tail domain from hemagglutinin (HA) of H5 A/Indonesia/5/05 (H5 Indo) (“H5iCT”, construct 9272); lane 6, a modified S protein with an ectodomain and transmembrane domain from OC43-CoV and a cytosolic tail domain from hemagglutinin (HA) of H5 A/Indonesia/5/05 (H5 Indo) with a variable margin between the OC43-CoV transmembrane (TM) domain and H5 A/Indonesia/5/05 HA cytosolic tail (CT) domain (“H5iCT (V4)”, construct 9273); lane 7, a modified S protein with an ectodomain and transmembrane domain from a OC43-CoV and a cytosolic tail domain from hemagglutinin (HA) of H1 A/California/7/2009 (“H1cCT”, construct 9274). The primary antibody used for detection was anti-coronavirus OC43 spike protein from Antibodies-online (ABIN2754654, 1/1000. The secondary antibody used for detection was Goat anti-Rabbit from JIR (111-035-144, 1/10000). The modified S protein has a molecular weight of about 150 kDa.

FIG. 23B shows an electron micrograph of virus like particles (VLP) comprising modified OC43-CoV S protein (with GGSGS+2P substitution) having a TMCT from H5 A/Indonesia/5/05 HA (H5iTMCT, construct 9270). FIG. 23C shows an electron micrograph of virus like particles (VLP) comprising modified OC43-CoV S protein (with GGSGS+2P substitution) having a cytoplasmic tail from H5 A/Indonesia/5/05 HA (H5iCT, construct 9272). FIG. 23D shows an electron micrograph of virus like particles (VLP) comprising modified OC43-CoV S protein (with GGSGS+2P substitution) having a cytoplasmic tail from H5 A/Indonesia/5/05 HA with a variable margin between the OC43-CoV transmembrane (TM) domain and H5 A/Indonesia/5/05 HA cytosolic tail (CT) domain (H5iCT (V4), construct 9273). FIG. 23E shows an electron micrograph of virus like particles (VLP) comprising modified OC43-CoV S protein (with GGSGS+2P substitution) having a cytoplasmic tail from H1 A/California/7/2009 HA (H1cCT, construct 9274).

FIG. 24A shows a schematic representation of vector 9269. FIG. 24B shows a schematic representation of vector 9270. FIG. 24C shows a schematic representation of vector 9272. FIG. 24D shows a schematic representation of vector 9273. FIG. 24E shows a schematic representation of vector 9274.

FIG. 25A shows an electron micrograph of virus like particles (VLP) comprising 229E-CoV S protein (with R567A+2P substitution) with native TMCT domain (wtTMCT, construct 9310). FIG. 25B shows an electron micrograph of virus like particles (VLP) comprising modified 229E-CoV S protein (with R567A+2P substitution) having a TMCT from H5 A/Indonesia/5/05 HA (H5iTMCT, construct 9311). FIG. 25C shows an electron micrograph of virus like particles (VLP) comprising modified 229E-CoV S protein (with R567A+2P substitution) having a cytoplasmic tail from H5 A/Indonesia/5/05 HA (H5iCT, construct 9312). FIG. 25D shows an electron micrograph of virus like particles (VLP) comprising modified 229E-CoV S protein (with R567A+2P substitution) having a cytoplasmic tail from H5 A/Indonesia/5/05 HA with a variable margin between the 229E-CoV transmembrane (TM) domain and H5 A/Indonesia/5/05 HA cytosolic tail (CT) domain (H5iCT (V4), construct 9313). FIG. 25E shows an electron micrograph of virus like particles (VLP) comprising modified 229E-CoV S protein (with R567A+2P substitution) having a cytoplasmic tail from H1 A/California/7/2009 HA (H1cCT, construct 9314).

FIG. 26A shows a schematic representation of vector 9310. FIG. 26B shows a schematic representation of vector 9311. FIG. 26C shows a schematic representation of vector 9312. FIG. 26D shows a schematic representation of vector 9313. FIG. 26E shows a schematic representation of vector 9314.

### DETAILED DESCRIPTION

The following description is of a preferred embodiment.

As used herein, the terms “comprising,” “having,” “including” and “containing,” and grammatical variations thereof, are inclusive or open-ended and do not exclude additional, un-recited elements and/or method steps. The term “consisting essentially of” when used herein in connection with a use or method, denotes that additional elements and/or method steps may be present, but that these additions do not materially affect the manner in which the recited method or use functions. The term “consisting of” when used herein in connection with a use or method, excludes the presence of additional elements and/or method steps. A use or method described herein as comprising certain elements and/or steps may also, in certain embodiments, consist essentially of those elements and/or steps, and in other embodiments consist of those elements and/or steps, whether or not these embodiments are specifically referred to. In addition, the use of the singular includes the plural, and “or” means “and/or” unless otherwise stated. The term “plurality” as used herein means more than one, for example, two or more, three or more, four or more, and the like. Unless otherwise defined herein, all technical and scientific terms used herein have the same meaning as commonly understood by one of ordinary skill in the art. As used herein, the term “about” refers to an approximately +/−10% variation from a given value. It is to be understood that such a variation is always included in any given value provided herein, whether or not it is specifically referred to. The use of the word “a” or “an” when used herein in conjunction with the term “comprising” may mean “one,” but it is also consistent with the meaning of “one or more,” “at least one” and “one or more than one.”

The present description relates to modified viral structural protein and their production in a host or host cell. The modified viral structural protein comprises in series, an ectodomain, a transmembrane domain (TM) or portion of a TM, and a cytosolic tail (CT) domain or portion of a CT, wherein the ectodomain and the TM or portion of the TM are derived from a Coronaviridae, and the CT or portion of the CT is derived from an influenza hemagglutinin (HA) protein.

The modified viral structural protein may be a modified Coronavirus structural protein, wherein the cytosolic tail domain or portion of the cytosolic tail domain has been replaced with the cytosolic tail domain or portion of the cytosolic tail domain of an influenza hemagglutinin (HA) protein. For example, the modified viral structural protein may be a modified Coronavirus spike or surface (S) protein, wherein the cytosolic tail domain or portion of the cytosolic tail domain of the S protein has been replaced with the cytosolic tail domain or portion of the cytosolic tail domain of an influenza hemagglutinin (HA) protein.

The present disclosure provides modified viral structural protein, wherein the ectodomain and the transmembrane domain of the modified viral structural protein may be derived from the ectodomain and the transmembrane domain of a Coronavirus S protein and wherein the cytosolic tail domain is derived from the cytosolic tail domain of an influenza hemagglutinin (HA) protein.

The modified S-protein may be a chimeric modified S-protein or a chimeric S-protein. By “chimeric S-protein”, it is meant a protein or polypeptide that comprises amino acid sequences and/or protein domains or portions of protein domains from two or more than two sources that are fused as a single polypeptide. For example but not limited to, the ectodomain and the transmembrane domain (TM) or portion of the TM of the chimeric S-protein may be derived from a first viral structural protein, for example a Coronavirus S protein, and the cytoplasmic tail (CT) or portion of the CT may be derived from a second viral structural protein, for example the CT may be derived from influenza HA. Furthermore, the ectodomain may be derived from a first viral structural protein for example a first Coronavirus S protein, the TM or portion of the TM may be derived from a second viral structural protein, for example a second Coronavirus S protein and the CT or portion of the CT may be derived from a third viral structural protein, for example the CT may be derived from influenza HA. Accordingly, the modified S-protein or chimeric S-protein may comprise a chimeric transmembrane and cytosolic tail domain (TMCT).

The modified coronavirus S-protein may comprise, in series,
an ectodomain derived from a coronavirus S-protein,
a transmembrane and cytosolic tail domain (TMCT), wherein the TMCT may be a chimeric TMCT, that may comprise:
   a transmembrane domain (TM), wherein the TM or a portion of the TM may be derived from a coronavirus S-protein and
   a cytosolic tail (CT), wherein the CT or a portion of the CT is derived from an influenza hemagglutinin (HA) protein.

The TM or portion of the TM may directly be fused or joined to the CT or portion of the CT or the TM or portion of the TM may be fused or joined to the CT or portion of the CT by an intervening peptide sequence.

Furthermore, the TM may be a chimeric TM that may comprise a N terminal sequence derived from the coronavirus S-protein TM and a C terminal sequence derived from the influenza HA protein TM. The CT may be a chimeric CT that may comprise a N terminal sequence derived from the coronavirus S-protein CT and a C terminal sequence derived from the influenza HA protein CT.

Accordingly, the chimeric TMCT, may comprise a native coronavirus S-protein TM, a chimeric coronavirus S-protein/influenza HA TM, a native influenza HA CT, a chimeric influenza HA/coronavirus S-protein CT or a combination thereof. The chimeric coronavirus S-protein/influenza HA TM comprises sequences from the TM of coronavirus S-protein and sequences from the TM of influenza HA. Similarly, the chimeric influenza HA/coronavirus S-protein CT comprises sequences from the CT of influenza HA and sequences from the CT of coronavirus S-protein.

A “chimeric transmembrane and cytosolic tail domain” or “chimeric TMCT” refers to a TMCT that is not native to the coronavirus S-protein TMCT. The chimeric TMCT comprises sequences that are not found together in nature. Thus the TMCT may comprise sequences that are heterologous to the ectodomain of the coronavirus S-protein. The term “heterologous” refers to a sequence or domain originating from different biological or synthetic sources. For example, the chimeric TMCT may comprise a TM or portion of a TM that is derived from the same coronavirus S-protein as the ectodomain, i.e. the TM may be homologous to the ectodomain of the S-protein or the TM or portion of the TM may be derived from a different viral TM, for example a TM from a different coronavirus S-protein as the ectodomain, i.e. the TM may be heterologous to the ectodomain of the S-protein. The CT or portion of the CT may be derived from a CT that is heterologous to the ectodomain, the TM, or both the ectodomain and the TM of the modified S-protein.

The coronavirus S protein, the modified S protein or the ectodomain and the transmembrane domain or portion of the transmembrane domain of the modified coronavirus S protein may be derived from any member of the Coronaviridae family of viruses. For example the coronavirus S-protein, the modified S-protein or the ectodomain and the transmembrane domain of the modified coronavirus S-protein may for example be derived from a Coronavirus, such as an Alphacoronavirus (Alpha-CoV), a Betacoronavirus (Beta-CoV), a Gammacoronavirus (Gamma-CoV) or a Deltacoronavirus (Delta-CoV). For example, the Coronavirus may be an Alphacoronavirus (Alpha-CoV) or a Betacoronavirus (Beta-CoV). The Alphacoronavirus may be a Duvinacovirus, such as for example HCoV-229E (229E-CoV), or may be a Setracovirus, such as for example HCoV-NL63. In a preferred embodiment, the Coronavirus is a Betacoronavirus (Beta-CoV). The Betacoronavirus may be a lineage A Betacoronavirus, such as for example HCoV-OC43 (OC43-CoV) or HCoV-HKU1 (HKU1-CoV), a lineage B Betacoronavirus, such as for example SARS-CoV (also referred to as SARS-CoV-1) or SARS-CoV-2 and variants thereof or a lineage C Betacoronavirus, such as for example MERS-CoV.

The coronavirus S-protein, the modified S-protein or the ectodomain and the transmembrane domain or portion of the transmembrane domain of the modified coronavirus S-protein may further be derived from variants of the SARS-CoV-2 lineage, including but not limited to the B.1.1.7 strain (“Alpha” variant) (20I/501Y.V1, MW531680.1), the B.1.351 strain (“Beta” variant) (20H/501Y.V2), the P.1 strain (“Gamma” variant) (20J/501Y.V3), the B 1.617.2 strain (“Delta” variant), the B.1.525 strain, the B.1.429 strain (the “ETA” variant) or other variants of strains comprising mutations that arise naturally in the coronavirus S protein, or naturally occurring recombinant strains thereof.

In one embodiment the ectodomain and the transmembrane domain or portion of the transmembrane domain of the modified viral structural protein are derived from the spike protein (S) of a Coronavirus of the SARS-CoV-2 lineage (also referred to as SARS-CoV-2 variants). In other embodiments the ectodomain and the transmembrane domain or portion of the transmembrane domain of the modified viral structural protein are derived from the spike protein (S) of SARS-CoV-1, MERS-CoV, OC43-CoV or 229E-CoV or variants thereof.

With reference to modified viral structural protein, the term “modified” as used herein may refer to the replacement of the cytoplasmic tail domain (CT) or portion of the CT in a structural protein from Coronaviridae with the CT or portion of the CT of a heterologous virus. For example a modified viral structural protein may be a Coronavirus S protein wherein the CT or portion of the CT of the S protein has been replaced with the CT or portion of the CT of influenza hemagglutinin (HA).

Therefore the modified viral structural protein may be a modified coronavirus spike (S) protein comprising a transmembrane domain (TM) or portion of a TM, and a cytosolic tail (CT) or portion of a CT, wherein the CT or portion of the CT may be derived from an influenza hemagglutinin (HA) protein and wherein the TM or portion of the TM is heterologous to the CT or portion of the CT. Furthermore, the modified S protein comprises a transmembrane domain (TM) or portion of the TM, and a cytosolic tail (CT) or portion of the CT, wherein the CT or portion of the CT may be derived from an influenza hemagglutinin (HA) protein and wherein the CT or portion of the CT is heterologous to the TM or portion of the TM.

Therefore, in one aspect it is provided a modified coronavirus spike (S) protein comprising a transmembrane domain (TM) or portion of a TM, and a cytosolic tail (CT) or portion of a CT, wherein the CT or portion of the CT is derived from an influenza hemagglutinin (HA) protein and wherein the TM or portion of the TM is heterologous to the CT or portion of the CT. The modified coronavirus spike (S) protein is also referred to as modified S protein.

The cytoplasmic tail domain may also be referred to as “cytoplasmic tail”, “cytosolic tail”, “cytosolic tail domain”, “CT, “CTD”, “cytoplasmic domain”, “cytoplasm domain”, “CP, “CPD” or “C-terminal domain” and similar expressions. The cytoplasmic tail domain may also encompass portions of the cytoplasmic tail domain.

It has been found that the modified viral structural protein such as a modified S protein as disclosed herewith has improved characteristics as compared to the wild-type or unmodified viral structural protein (for example the S-protein). Examples of improved characteristics of the modified viral structural protein such as the modified S protein include but are not limited to: increased yield of the modified viral structural protein when expressed in a host or host cell as compared to the wild-type or unmodified viral structural protein; improved integrity, stability, or both integrity and stability, of the viral structural protein when expressed in a host or host cell as compared to the wild-type or unmodified viral structural protein; improved integrity, stability, or both integrity and stability, of virus like particles (VLPs) that are comprised of the modified viral structural protein as compared to the integrity, stability or integrity and stability of VLPs comprising to viral structural protein that does not comprise the modification as described herewith; increased yield of VLPs comprising modified viral structural protein when expressed in host cells as compared to the yield of VLPs that do not comprise the modified viral structural protein that are expressed in same or substantially similar host cells.

Furthermore, methods of producing virus like particle (VLP) comprising modified viral structural protein such as the modified S protein in a host or host cell are also described. It has been observed that when VLPs are produced that comprise a modified viral structural protein such as the modified S protein wherein the native or wild-type CT has been replaced with a CT of influenza HA as described herein, the yield of VLP production in a host is increased compared to the yield of VLP that comprise viral structural protein that either i) comprise the native CT or ii) comprise a modified viral structural protein wherein the transmembrane domain (TM) and the CT have been replaced with the TM and the CT of an influenza HA.

The transmembrane domain may also be referred to as “TM” or “TMD”. The transmembrane and cytoplasmic tail domain may be referred to as TMCT or TM/CT.

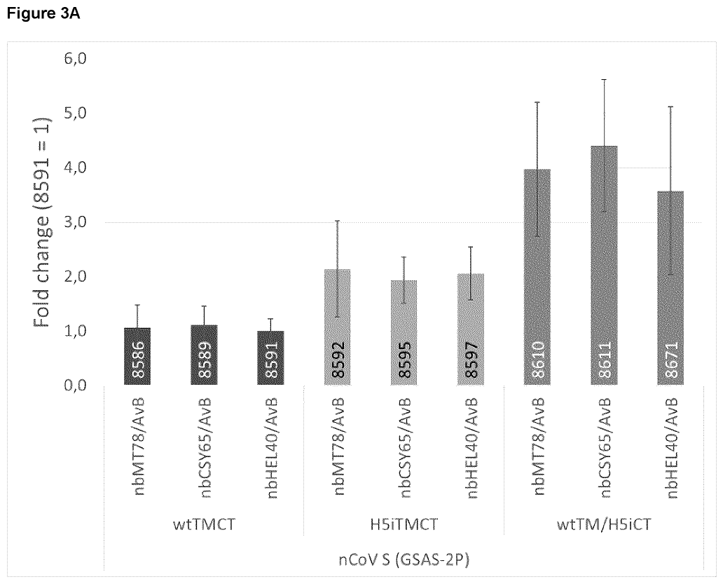

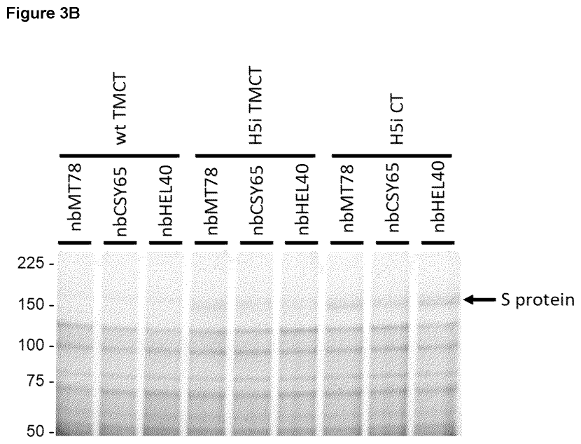

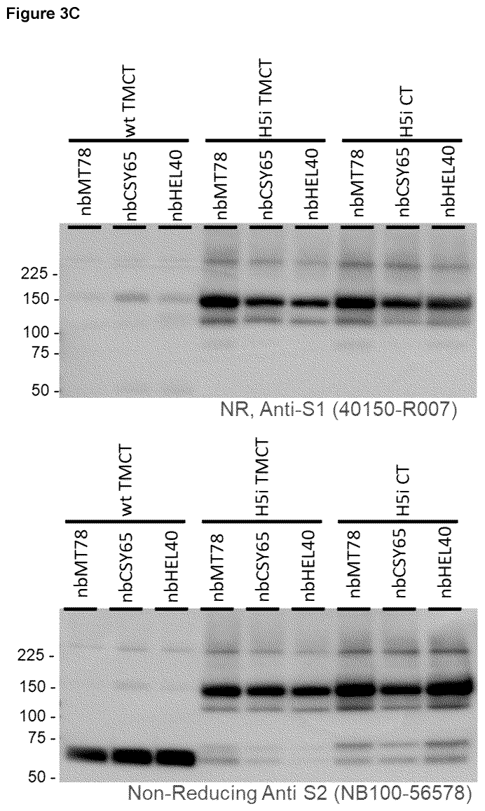

FIG. 3A shows that when a modified S protein (e.g. modified SARS-CoV-2 S-protein) was expressed in plants, the yield or protein accumulation (expressed as fold-change) of the modified S protein was increased approximately 2 fold when the native transmembrane and cytoplasmic tail (TMCT) was replaced with a TMCT from influenza HA (constructs 8592, 8595, and 8597) compared to the yield or protein accumulation of S protein with native TMCT (constructs 8586, 8589, and 8591). Furthermore, when a modified S protein (e.g. modified SARS-CoV-2 S-protein) wherein only the cytoplasmic tail (CT) was replaced with the CT of influenza HA (constructs 8610, 8611, and 8671) was expressed in plants, the protein accumulation of the modified S protein with the CT of influenza HA (expressed as fold-change), further increased between approximately 1.74 to 2.14 times, as compared to accumulation of modified S protein wherein the TMCT had been replaced with the TMCT of influenza HA. Correspondingly, the protein accumulation of the modified S protein with the CT of influenza HA increased between approximately 3.57 to 4.40 times, as compared to accumulation of S protein with the native transmembrane and cytoplasmic tail (wtTMCT).

FIG. 3B shows that higher protein accumulation was observed for modified S protein (modified SARS-CoV-2 S-protein) with a cytoplasmic tail from influenza HA (H5i CT) when compared to protein accumulation of S protein with a wild-type TMCT (wt TMCT) or a modified S protein with the TMCT of influenza HA (H5i TMCT) from crude plant extract. Modified S protein with a cytoplasmic tail from influenza HA (H5i CT) is visible by Coomassie blue staining alone. The bands for modified S protein with a cytoplasmic tail from influenza HA (H5i CT) are more pronounced and thicker compared to the band of S protein with a wild-type TMCT (wt TMCT) or modified S protein with the TMCT of influenza HA (H5i TMCT)—see bands at about 150 kDa marked as S protein. Thickness of bands correspond to the amount of protein present, indicating that more protein accumulated for the H5i CT S protein. This higher protein accumulation was observed irrespective of the expression enhancer that was used.

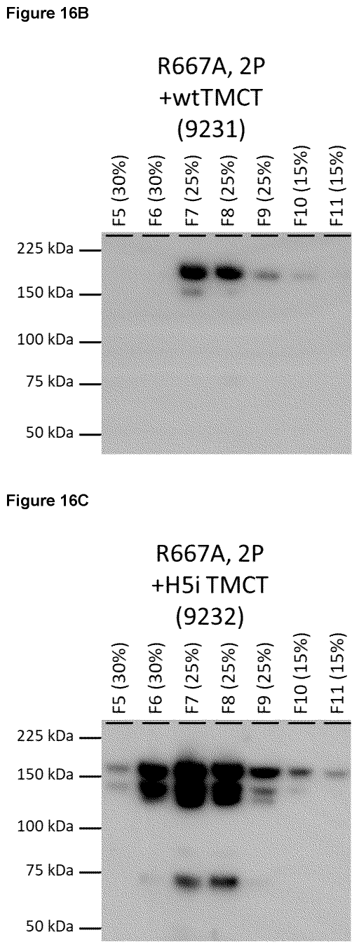

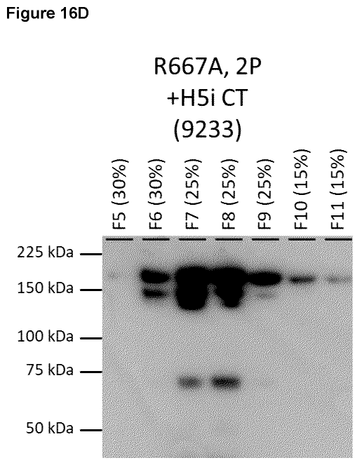

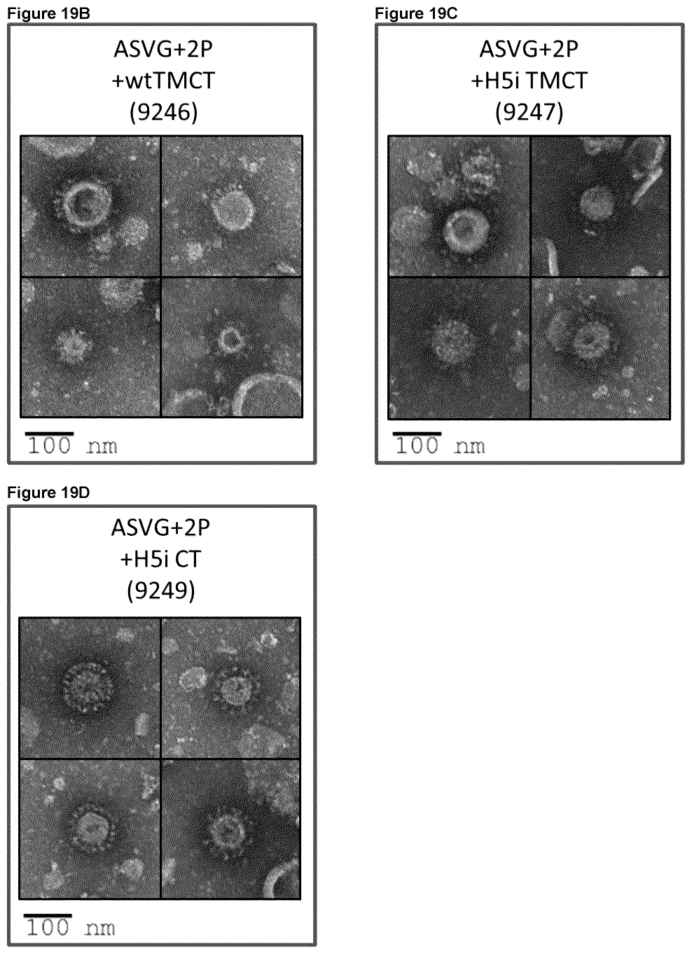

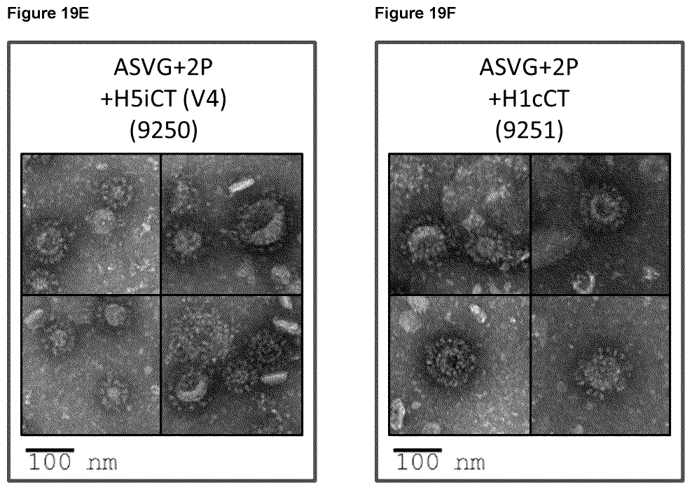

As further discussed in more detail below, similar results were obtained, wherein the modified S-protein comprises a SARS-CoV-1 S protein with a cytoplasmic tail from influenza HA (see FIG. 16A) or a MERS CoV S protein with a cytoplasmic tail from influenza HA (see FIG. 19A).

FIG. 3C shows S protein (SARS-CoV-2 S protein) accumulation by Western blot analysis of crude plant extract. When a modified S protein with a cytoplasmic tail from influenza HA (H5i CT) was expressed in plants, higher accumulation of modified S protein was observed compared to S protein with a wild-type TMCT (wt TMCT) and a modified S protein wherein both the transmembrane domain and the cytoplasmic tail (TMCT) domain have been replaced with the TMCT from influenza HA (H5i TMCT). The Western blot analysis in FIG. 3C further shows that the SARS CoV-2 S-protein comprises both an S1 domain/subunit (top panel, detection with anti-SARS-CoV-2 S1 antibody) and an S2 domain/subunit (bottom panel, detection with an anti-SARS-CoV-2 S2 antibody) and has a molecular weight of about 150 kDa.

The present description provides a modified viral structural protein, wherein the modified viral structural protein may be a modified Coronavirus Spike or Surface Protein (S protein). The modified S protein comprising, in series, an ectodomain, a transmembrane domain (TM) or portion of a TM, and a cytosolic tail (CT) domain or portion of a CT, wherein the ectodomain and the transmembrane domain are derived from Coronavirus, and the CT or portion of the CT is derived from a CT of influenza hemagglutinin (HA) protein. The ectodomain and the transmembrane domain or portion of the TM may be derived from the same Coronavirus. Therefore, the ectodomain and the transmembrane domain or portion of the TM of the modified structural protein are homologues (i.e. not heterologous) to each other, whereas the CT or portion of the CT is heterologous to the ectodomain and the transmembrane domain.

Furthermore, the transmembrane domain (TM) or portion of the TM of the modified S protein may be derived from a different Coronavirus than the ectodomain. Therefore, the TM or portion of the TM in the modified S protein may be heterologous (not homologous) to both the ectodomain and the CT domain or portion of the CT of the modified S protein. Similarly, the ectodomain may be heterologous (not homologous) to the TM or portion of the TM and the CT domain or portion of the CT of the modified S protein. For example, the ectodomain of the modified S protein may be derived from a first Coronavirus, the TM or portion of the TM may be derived from a second Coronavirus and the CT or portion of the CT may be derived from an influenza HA. The first Coronavirus and the second Coronavirus may belong to different Coronavirus families, sub-groups, types, subtypes, lineages or strains. The first Coronavirus and second Coronavirus may therefore be heterologous to each other and also each heterologous to the virus family from which the CT or portion of the CT is derived.

For example, the first Coronavirus from which the S protein ectodomain is derived, may be from any Coronavirus such for example an Alphacoronavirus (Alpha-CoV) or a Betacoronavirus (Beta-CoV). A non-limiting example of the first coronavirus from which the ectodomain of the S protein may be derived is a Duvinacovirus, such for example HCoV-229E, a Setracovirus, such for example HCoV-NL63. a lineage A Betacoronavirus, such for example HCoV-OC43 or HCoV-HKU1, a lineage B Betacoronavirus, such for example SARS-CoV or SARS-CoV 2 or a lineage C Betacoronavirus such for example MERS-CoV. The second Coronavirus, from which the TM is derived, may belong to a different Coronavirus family, sub-group, type, subtype, lineage or strain than the first Coronavirus from which the ectodomain is derived. For example the second Coronavirus from which the S protein TM is derived, may be from any Coronavirus such for example an Alphacoronavirus (Alpha-CoV) or Betacoronavirus (Beta-CoV), as long as the second Coronavirus is heterologous to the first Coronavirus. A non-limiting example of the second coronavirus from which the TM of the S protein may be derived is a Duvinacovirus, such for example HCoV-229E (also referred to as 229E-CoV), a Setracovirus, such for example HCoV-NL63 (NL63-CoV), a lineage A Betacoronavirus, such for example HCoV-OC43 (also referred to as OC43-CoV) or HCoV-HKU1 (HKU1-CoV), a lineage B Betacoronavirus, such for example SARS-CoV (also referred to as SARS-CoV 1) or SARS-CoV 2 or a lineage C Betacoronavirus such for example MERS-CoV (also simply referred to as “MERS”).

The domains in a Coronavirus S protein, such as the SARS-CoV-1 S-protein, SARS-CoV-2 S-protein, MERS CoV S-protein, OC43-CoV S-protein, or 229E-CoV S-protein, may readily be identified by methods known within the art. For example, domains such as transmembrane domains, may be identified by determining the degree of hydrophobicity of an amino acid sequence of the protein, for example using a transmembrane prediction program (e.g. Expert Protein Analysis System; ExPASy.org, operated by the Swiss Institute of Bioinformatics; or the Dense Alignment Surface Method, Cserzo M., et al. 1997, Prot. Eng. vol. 10, no. 6, 673-676; Lolkema J. S. 1998, FEMS Microbiol Rev. 22, no 4, 305-322), by determining the hydropathy profile of the amino acid sequence of the protein (e.g. Kyte-Doolittle Hydropathy Profile), by determining the three-dimensional protein structure and identifying the structure that is thermodynamically stable in a membrane (e.g. a single alpha helix, a stable complex of several transmembrane alpha helices, a transmembrane beta barrel, a beta-helix, or any other structure that is thermodynamically stable in a membrane).

Furthermore, domains within a Coronavirus S protein may be determined by comparison to known protein sequences for example by sequence alignment. Methods of alignment of sequences for comparison are well-known in the art and as further described below.

Domains and domain organization of Coronavirus S protein are well known and have been described. All Coronavirus spike proteins (S protein) share the same organization in two subunits or domains: a N-terminal subunit (or domain) named S1 that is responsible for receptor binding and a C-terminal S2 subunit (or domain) responsible for virus attachment, membrane fusion and virus entry.

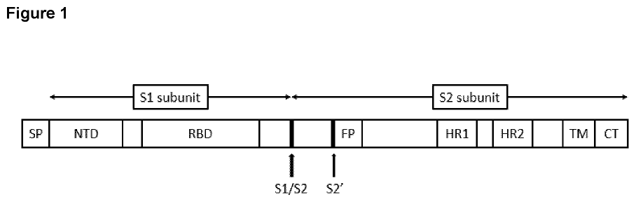

FIG. 1A shows a schematic representation of the Coronavirus S protein with its subunits and domains and the location of the S1/S2 and S2′ cleavage sites. The S1 subunit is distal to the virus membrane and contains the receptor-binding domain (RBD) that mediates virus attachment to its host receptor. The S2 subunit contains fusion protein machinery, such as the fusion peptide, two heptad-repeat sequences (HR1 and HR2), a central helix typical of fusion glycoproteins and a transmembrane domain, and the cytosolic tail domain (see for example Kirchdoerfer et al. Nature 2016 Mar. 3; 531(7592):118-2, which is herein incorporated by reference).

The transmembrane domain (TM) and the cytoplasmic tail domain (CT) are positioned at the C-terminal end of the S2 subunit. While these domains are conserved in all coronaviruses (see FIG. 1A and Corver et al. 2009, Virol J. 2009; 6: 230, which is herein incorporated by reference), different references, groups and authors have referred to different amino acid numbering with respect to these domains.

For example, amino acids (aa): 1214-1234 may be assigned to the TM and aa 1235-1273 may be assigned to the CT in the S protein of SARS-CoV-2 (see for example UniProtKB-P0DTC2 (SPIKE_SARS2)). When aligning the sequence of SARS-CoV-2 (SEQ ID NO. 1) with the sequence of SARS-CoV-1 of Kirchdoerfer et al. (Nature 2016 Mar. 3; 531(7592):118-2) the SARS-CoV-2 TM corresponds to amino acids 1214-1236 and the SARS-CoV-2 CT corresponds to amino acids: 1237-1273.

For the purpose of this disclosure, the TM and CT of the native (unmodified) S protein corresponds to the following amino acids when aligned to a Coronavirus S protein reference sequence (SEQ ID NO: 1): TM: amino acids 1214-1234 and CT: amino acids: 1235-1273.

When 15 amino acids comprising the signal peptide (SP) are removed from the S protein, the TM corresponds to amino acids 1199-1219 of reference sequence SEQ ID NO: 2 and the CT corresponds to amino acids 1220-1258 of SEQ ID NO:2. (see also Table 1 for reference sequences and numbering).

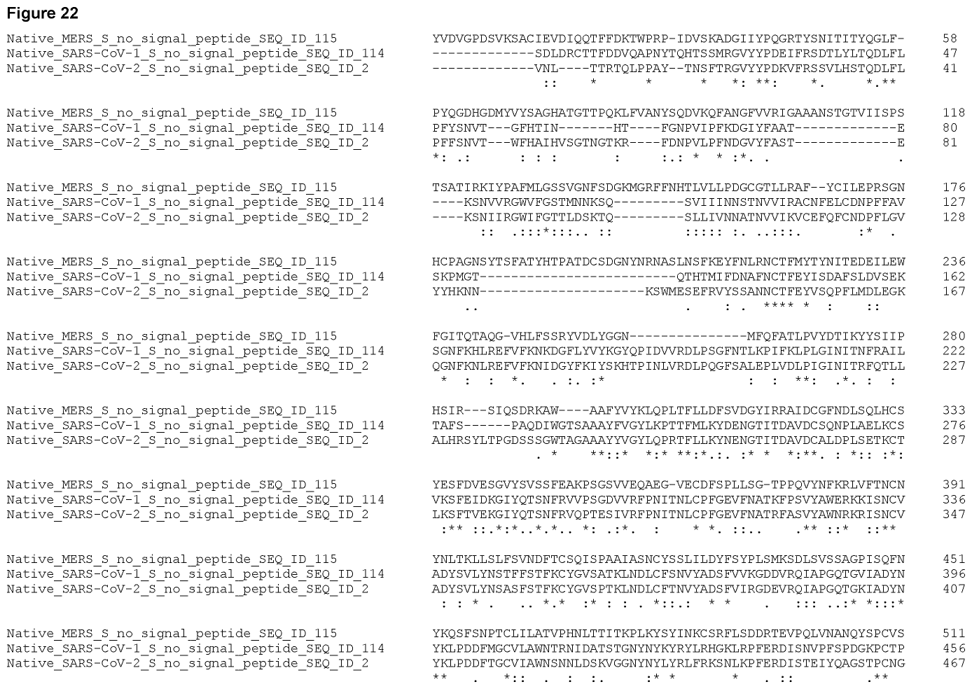

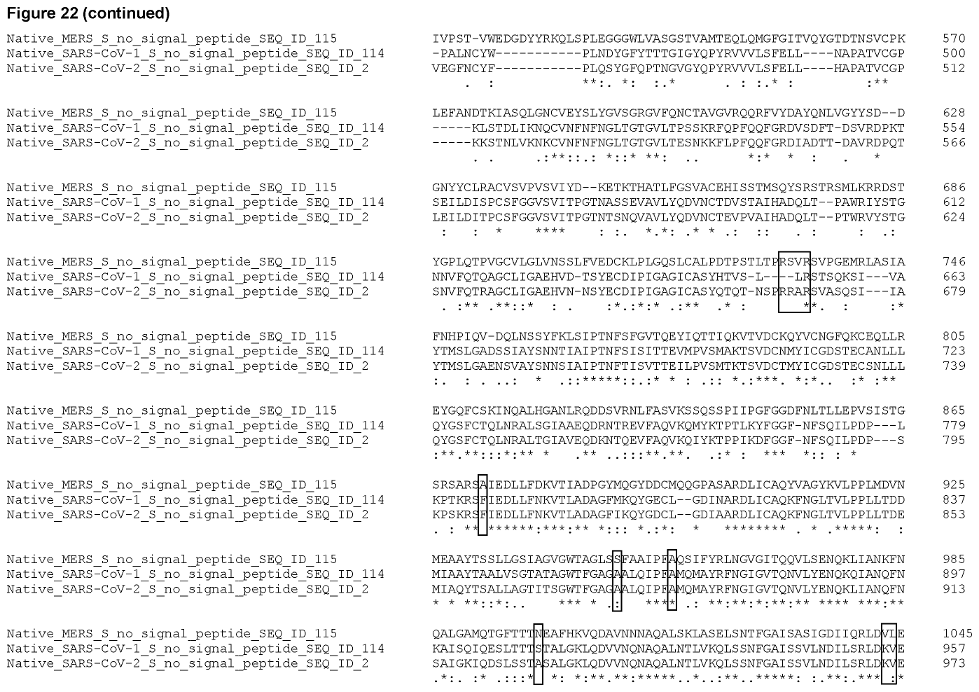

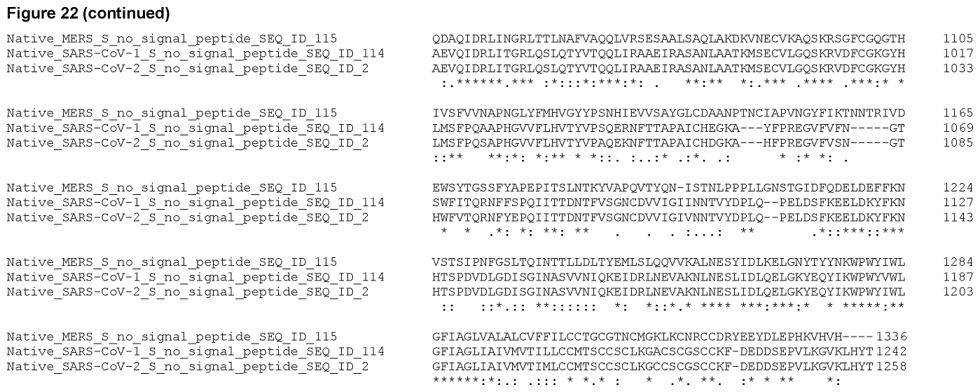

The TM of Coronavirus S-protein has a highly conserved N-terminal aromatic rich stretch, followed by a hydrophobic sequence (see FIG. 22 and Corver et al. Virology Journal volume 6, 230 (2009)). The consensus sequence of Coronavirus S-protein TM domain is:

(SEQ ID NO: 132)
WYXWLGFIAGLXAXXX{X}VXXXL, (wherein {X} may be
absent).

For example, the Coronavirus S-protein TM domain consensus sequence may be:

(SEQ ID NO: 133)
WY[I/V]WLGFIAGL[V/I]A[L/I][A/V][L/M]{X}V[F/T][F/I]
XL, (wherein {X} may be C or absent).

TABLE 1
Non-limiting examples of positions of TM and CT
domains in modified S protein and corresponding
amino acid positions in reference sequences.

| | Transmembrane | Cystoplasmic Tail |
|---|---|---|
| S Protein | Domain (TM) | Domain (CT) |
| Modified S Protein¹ | 1199-1219 | 1220-1235 |
| [SARS-CoV-2 H5iCT] | | |
| (SEQ ID NO: 21) | | |
| SARS-CoV-2² | 1214-1234 | 1235-1273 |
| (SEQ ID NO. 1) | | |
| SARS-CoV-2³ | 1199-1219 | 1220-1258 |
| (SEQ ID NO: 2) | | |
| SARS-CoV | 1196-1216 | 1217-1255 |
| (SARS-CoV-1)² | | |
| (SEQ ID NO: 112) | | |
| SARS-CoV | 1183-1203 | 1204-1242 |
| (SARS-CoV-1)³ | | |
| (SEQ ID NO: 114) | | |
| MERS | 1297-1318 | 1319-1353 |
| (MERS-CoV)² | | |
| (SEQ ID NO: 113) | | |
| MERS | 1280-1301 | 1302-1336 |
| (MERS-CoV)³ | | |
| (SEQ ID NO: 115) | | |
| OC43-CoV² | 1233-1325 | 1326-1360 |
| (SEQ ID NO: 158) | | |
| OC43-CoV³ | 1291-1311 | 1312-1346 |
| (SEQ ID NO: 160) | | |
| 229E-CoV² | 1116-1135 | 1136-1173 |
| (SEQ ID NO: 159) | | |
| 229E-CoV³ | 1100-1119 | 1120-1157 |
| (SEQ ID NO: 161) | | |

¹numbering excludes signal peptide, CT is derived from influenza HA
²numbering includes signal peptide, CT is native
³numbering excludes signal peptide, CT is native

While there are differences in the numbering of the residues assigned to the TM and CT domain, a person of skill in the art will be able to determine the borders or boundaries of these domains in a Coronavirus S-protein by using known methods as for example described below.

In the modified Coronavirus S protein, the heterologous CT or portion of the CT that may be derived from influenza HA may be directly fused to the C-terminal end of the TM or portion of the TM of the Coronavirus S protein, or the heterologous CT or portion of the CT may be fused to the C-terminal end of the TM or portion of the TM of the Coronavirus S protein with an intervening peptide sequence (also referred to as a linker or linker sequence). Accordingly, the modified S-protein may comprise a intervening peptide, wherein the intervening peptide sequence fuses the CT or portion of the CT to the C-terminal end of the TM or portion of the TM.

The heterologous CT, portion of the CT or the intervening peptide sequence with the heterologous CT may be fused to an amino acid in the C-terminal portion of the TM domain (for example within 4 amino acids of the C-terminus of the TM domain as defined in Table 1 with reference to SEQ ID NO: 1, 2, 21, 114, 115, 160 or 161) or the N-terminal portion of the CT domain (for example within 4 amino acids of the N-terminus of the CT domain as defined in Table 1 with reference to SEQ ID NO: 1, 2, 21, 114, 115, 160 or 161).

For example, the Coronavirus TM may end at an amino acid residue that corresponds to any one of amino acids 1230-1238 of SEQ ID NO: 1. Accordingly, the C-terminal end of the Coronavirus TM may be an amino acid that corresponds to any one of amino acids 1230-1238 of SEQ ID NO: 1. In one example the Coronavirus TM may end at an amino acid residue that corresponds to amino acid 1230 in SEQ ID NO: 1. In another example, the TM may end at an amino acid residue that corresponds to amino acid 1231 in SEQ ID NO: 1. In a further example, the TM may end at an amino acid residue that corresponds to amino acid 1232 in SEQ ID NO: 1. In another example, the TM may end at an amino acid residue that corresponds to amino acid 1233 in SEQ ID NO: 1. In a further example, the TM may end at an amino acid residue that corresponds to amino acid 1234 in SEQ ID NO: 1. In another example, the TM may end at an amino acid residue that corresponds to amino acid 1235 in SEQ ID NO: 1. In another example, the TM may end at an amino acid residue that corresponds to amino acid 1236 in SEQ ID NO: 1. In another example, the TM may end at an amino acid residue that corresponds to amino acid 1237 in SEQ ID NO: 1. In another example, the TM may end at an amino acid residue that corresponds to amino acid 1238 in SEQ ID NO: 1. In a preferred embodiment the TM may end at an amino acid residue that corresponds to amino acid 1234 in SEQ ID NO: 1.

In another example, the Coronavirus TM or portion of the TM may end at an amino acid residue that corresponds to any one of amino acids 1215-1219 of SEQ ID NO: 2 or 21. Accordingly, the C-terminal end of the Coronavirus TM or portion of the TM may be an amino acid that corresponds to any one of amino acids 1215-1224 of SEQ ID NO: 2 or 21. In one example the Coronavirus TM or portion of the TM may end at an amino acid residue that corresponds to amino acid 1215 in SEQ ID NO: 2 or 21. In another example, the TM or portion of the TM may end at an amino acid residue that corresponds to amino acid 1216 in SEQ ID NO: 2 or 21. In a further example, the TM or portion of the TM may end at an amino acid residue that corresponds to amino acid 1217 in SEQ ID NO: 2 or 21. In another example, the TM or portion of the TM may end at an amino acid residue that corresponds to amino acid 1218 in SEQ ID NO: 2 or 21. In another example, the TM or portion of the TM may end at an amino acid residue that corresponds to amino acid 1219 in SEQ ID NO: 2 or 21.

The intervening peptide sequence that may fuse the heterologous CT to the C-terminal end of the TM or portion of the TM from the Coronavirus S protein may have a length from 0-10 amino acids. Accordingly, the intervening peptide sequence may have a length of 0, 1, 2, 3, 4, 5, 6, 7, 8, 9 or 10 amino acids. The intervening peptide sequence may be derived from a Coronavirus protein, for example the intervening peptide sequence may be derived from the C-terminal end of the TM from a Coronavirus S protein or from the N-terminal end of the CT of a Coronavirus S protein or both. The intervening peptide sequence may further be derived from an influenza HA protein, for example the intervening peptide sequence may be derived from the C-terminal end of the TM from influenza HA protein or from the N-terminal end of the CT of influenza HA protein, or both. Furthermore, the intervening peptide sequence may be heterologous to the Coronavirus and/or the HA portion of the modified S protein or the intervening peptide sequence may be an artificial sequence.

Non-limiting examples of sequences of the TM/CT domain (also referred to as chimeric TMCT) of the modified S protein are as shown below. The sequence of the TM domain from Coronavirus S-protein is underlined and the CT domain derived from influenza HA is shown in bold. Sequences in italic and bold are sequences derived from the TM of influenza HA. Sequences in italic and underlined are sequences derived from the CT of Coronavirus S-protein.

SARS-CoV-2
(SEQ ID NO: 18)
WYIWLGFIAGLIAIVMVTIMLSLWMCSNGSLQCRICI (wtTM/H5iCT)
(SEQ ID NO: 19)
WYIWLGFIAGLIAIVMVTIMMAGLSLWMCSNGSLQCRICI (wtTM/
H5iCT V1)
(SEQ ID NO: 37)
WYIWLGFIAGLIAIVMVTIMAGLSLWMCSNGSLQCRICI (wtTM/
H5iCT V2)
(SEQ ID NO: 38)
WYIWLGFIAGLIAIVMVTIMLCCMCSNGSLQCRICI (wtTM/H5ICT
V3)
(SEQ ID NO: 39)
WYIWLGFIAGLIAIVMVTIMLCCSNGSLQCRICI (wtTM/H5iCT V4)
(SEQ ID NO: 126)
WYIWLGFIAGLIAIVMVTIMLSFWMCSNGSLQCRICI (wtTM/HliCT)
(SEQ ID NO: 127)
WYIWLGFIAGLIAIVMVTIMLMWACQKGNIRCNICI (wtTM/H3iCT)
(SEQ ID NO: 128)
WYIWLGFIAGLIAIVMVTIMLGLWMCSNGSMQCRICI (wtTM/H6iCT)
(SEQ ID NO: 129)
WYIWLGFIAGLIAIVMVTIMLVFICVKNGNMRCTICI (wtTM/H7iCT)
(SEQ ID NO: 130)
WYIWLGFIAGLIAIVMVTIMLLEWAMSNGSCRCNICI (wtTM/H9iCT)
(SEQ ID NO: 131)
WYIWLGFIAGLIAIVMVTIMLVVYMVSRDNVSCSICL (wtTM/BiCT)
SARS-CoV-1
(SEQ ID NO: 118)
WYVWLGFIAGLIAIVMVTILLSLWM CSNGSLQCRICI (wtTM/
H5iCT)
(SEQ ID NO: 119)
WYVWLGFIAGLIAIVMVTILLCCSNGSLQCRICI (wtTM/H5ICT V4)
(SEQ ID NO: 120)
WYVWLGFIAGLIAIVMVTILLSFWM CSNGSLQCRICI (wtTM/
H1cCT)
MERS-COV
(SEQ ID NO: 123)
WYIWLGFIAGLVALALCVFFILSLWMCSNGSLQCRICI (wtTM/
H5iCT)
(SEQ ID NO: 124)
WYIWLGFIAGLVALALCVFFILCCSNGSLQCRICI (wtTM/H5iCT
V4)
(SEQ ID NO: 125)
WYIWLGFIAGLVALALCVFFILSFWMCSNGSLQCRICI (wtTM/
H1cCT)
OC43-CoV
(SEQ ID NO: 164)
WYVWLLICLAGVAMLVLLFFISLWMCSNGSLQCRICI (wtTM/H5iCT)
(SEQ ID NO: 165)
WYVWLLICLAGVAMLVLLFFICCSNGSLQCRICI (wtTM/H5ICT V4)
(SEQ ID NO: 166)
WYVWLLICLAGVAMLVLLFFISFWMCSNGSLQCRICI (wtTM/
H1cCT)
229E-CoV
(SEQ ID NO: 169)
WWVWLCISVVLIFVVSMLLLSLWMCSNGSLQCRICI (wtTM/H5iCT)
(SEQ ID NO: 170)
WWVWLCISVVLIFVVSMLLLCCSNGSLQCRICI (wtTM/H5ICT V4)
(SEQ ID NO: 171)
WWVWLCISVVLIFVVSMLLLSFWMCSNGSLQCRICI (wtTM/H1cCT)

The modified coronavirus S-protein may comprise a chimeric TMCT. For example, the chimeric TMCT may comprise N-terminal sequences derived from coronavirus S-protein and C-terminal sequence derived from influenza HA protein as indicated in Table 1B. The TM may comprise the sequences as indicated in the column labeled as “S-protein TM sequences” and the CT may comprise the sequences as indicated in the column labeled as “HA protein CT sequence”. The CT and TM may be joined by the sequences as indicated in columns labeled as “S-protein CT sequence” and/or “HA protein TM sequence” (also referred to as intervening sequences or linker, as further described below).

TABLE 1B
Non-limiting examples of TM and CT sequences in
modified S protein. The amino acid positions
within the reference sequences are indicated.

| SEQ | S-protein | S-protein | HA protein | HA protein |
|---|---|---|---|---|
| ID NO: | TM sequence | CT sequence | TM sequence | CT sequence |
| 18 | 1-21 | — | — | 22-37 |
| 19 | 1-20 | — | 21-24 | 25-40 |
| 37 | 1-20 | — | 21-23 | 24-39 |
| 38 | 1-21 | 22-24 | — | 25-36 |
| 39 | 1-21 | 22-23 | — | 24-34 |
| 126 | 1-21 | | | 22-37 |
| 127 | 1-21 | | | 22-36 |
| 128 | 1-21 | | | 22-37 |
| 129 | 1-21 | | | 22-37 |
| 130 | 1-21 | | | 22-37 |
| 131 | 1-21 | | | 22-37 |
| 118 | 1-21 | | | 22-37 |
| 119 | 1-21 | 22-23 | — | 24-34 |
| 120 | 1-21 | | | 22-37 |
| 123 | 1-22 | | | 23-38 |
| 124 | 1-22 | 23-24 | — | 25-35 |
| 125 | 1-22 | | | 23-38 |
| 164 | 1-21 | | | 22-37 |
| 165 | 1-21 | 22-23 | — | 24-34 |
| 166 | 1-21 | | | 22-37 |
| 169 | 1-20 | | | 21-36 |
| 170 | 1-20 | 21-22 | — | 23-33 |
| 171 | 1-20 | | | 21-36 |

For example, the N-terminal sequence derived from coronavirus S-protein TM may comprise at least the following:
at least 19 amino acids corresponding to amino acids 1-19 of SEQ ID NO: 18, 19, 37, 38, 39, 118, 119, 123, 124, 164, 165, 169 or 170;
at least 20 amino acids corresponding to amino acids 1-20 of SEQ TD NO: 18, 19, 37, 38, 39, 118, 119, 123, 124, 164, 165, 169 or 170;
at least 21 amino acids corresponding to amino acids 1-21 of SEQ ID NO: 18, 19, 37, 38, 39, 118, 119, 123, 124, 164, 165, 169 or 170;
at least 22 amino acids corresponding to amino acids 1-22 of SEQ ID NO: 18, 19, 37, 38, 39, 118, 119, 123, 124, 164, 165, 169 or 170;
at least 23 amino acids corresponding to amino acids 1-23 of SEQ ID NO: 18, 19, 37, 38, 39, 118, 119, 123, 124, 164, 165, 169 or 170;
at least 24 amino acids corresponding to amino acids 1-24 of SEQ ID NO: 18, 19, 37, 38, 39, 118, 119, 123, 124, 164, 165, 169 or 170.

The N-terminal sequence derived from the coronavirus S-protein TM may comprise at least 20 amino acids corresponding to amino acids 1-20 of SEQ ID NO: 18 or 169, or at least 21 amino acids corresponding to amino acids 1-21 of SEQ ID NO: 118 or 164, or at least 22 amino acids corresponding to amino acids 1-22 of SEQ ID NO: 123 and one or more than one amino acid from the C-terminal end of the influenza HA protein TM. The N-terminal sequence derived from the coronavirus S-protein TM may comprise at least 20 amino acids corresponding to amino acids 1-20 of SEQ ID NO: 18 or 169, or at least 21 amino acids corresponding to amino acids 1-21 of SEQ ID NO: 118 or 164, or at least 22 amino acids corresponding to amino acids 1-22 of SEQ ID NO: 123 and one or more than one amino acid from the C-terminal end of the influenza HA protein TM. The one or more than one amino acid from the C-terminal end of the influenza HA protein TM may comprise 0, 1, 2, 3, 4, 5, 6, 7, 8, 9 or 10 amino acids. For example the one or more than one amino acid may be 2, 3 or 4 amino acids. The one or more than one amino acid from the C-terminal end of the influenza HA protein TM may be A, C, G, L, S, M, W or conserved substitution of A, C, G, L, S, M, W, or a combination thereof. In one example the one or more than one amino acid from the C-terminal end of the influenza HA protein TM may be selected from AG or conserved substitution of AG, AGL or conserved substitution of AGL, MAGL or conserved substitution of MAGL.

The modified coronavirus S-protein may also comprise a chimeric CT comprising a N-terminal sequence derived from the coronavirus S-protein CT and a C-terminal sequence derived from the influenza HA protein CT.

The N-terminal sequence derived from the coronavirus S-protein CT may comprise one or more than one amino acid. The one or more than one amino acid from the N terminal end of the coronavirus S-protein CT may comprise 0, 1, 2, 3, 4 or 5 amino acids. For example the one or more than one amino acid may be 1, 2 or 3 amino acids. The one or more than one amino acid from the N-terminal end of the coronavirus S-protein CT may be C or M or conserved substitutions of C or M. In one example the one or more than one amino acids from the N-terminal end of the coronavirus S-protein may be selected from C or a conserved substitution of C, CC or a conserved substitution of CC, or CCM or a or a conserved substitution of CCM.

The C-terminal sequence derived from the influenza HA protein CT may comprise at least 11 amino acids corresponding to amino acids 27-37 of SEQ ID NO: 18.

The N-terminal sequence derived from the influenza HA protein CT may further comprise at least 12 amino acids corresponding to amino acids 26-37 of SEQ ID NO: 18, at least 13 amino acids corresponding to amino acids 25-37 of SEQ ID NO: 18, at least 14 amino acids corresponding to amino acids 24-37 of SEQ ID NO: 18, at least 15 amino acids corresponding to amino acids 23-37 of SEQ ID NO: 18, or at least 16 amino acids corresponding to amino acids 22-37 of SEQ ID NO: 18.

In another example, the C-terminal sequence derived from the influenza HA protein CT may comprise at least the following:
at least 11 amino acids corresponding to amino acids 27-37 of SEQ ID NO: 126, 127, 128, 129, 130 or 131;
at least 12 amino acids corresponding to amino acids 26-37 of SEQ ID NO: 126, 127, 128, 129, 130 or 131;
at least 13 amino acids corresponding to amino acids 25-37 of SEQ ID NO: 126, 127, 128, 129, 130 or 131;
at least 14 amino acids corresponding to amino acids 24-37 of SEQ ID NO: 126, 127, 128, 129, 130 or 131;
at least 15 amino acids corresponding to amino acids 23-37 of SEQ ID NO: 126, 127, 128, 129, 130 or 131; or
at least 16 amino acids corresponding to amino acids 22-37 of SEQ ID NO: 126, 127, 128, 129, 130 or 131.

For example the CT may comprise the sequences as indicated in Table 1B (HA protein CT sequence). For example the CT may comprise amino acids 22-37 of SEQ ID NO: 18, 126, 128, 129, 130, 131, 118, 120, 164 or 166; or amino acids 25-40 of SEQ ID NO: 19; or amino acids 24-39 of SEQ ID NO: 37; or amino acids 25-36 of SEQ ID NO: 38; or amino acids 24-34 of SEQ ID NO: 39 or 119; or amino acids 22-36 of SEQ ID NO: 127; or amino acids 22-37 of SEQ ID NO: 118 or 164; or amino acids 23-38 of SEQ ID NO: 123 or 125; or amino acids 25-35 of SEQ ID NO: 124; or amino acids 24-34 of SEQ ID NO: 165; or amino acids 21-36 of SEQ ID NO: 169; or amino acids 23-33 of SEQ ID NO: 170; or amino acids of SEQ ID NO: 21-36.

The influenza CT or portion of the CT may be fused or joined to the TM or portion of the TM of the S-protein with an intervening peptide sequence. For example, the intervening peptide sequence may be derived from the influenza CT, the S-protein TM or a combination thereof or the intervening peptide sequence may be an artificial sequence. The intervening peptide sequence may be of varying length. For example, the intervening peptide sequence may be 1, 2, 3, 4, 5, 6, 7, 8, 9 or 10 amino acid long, preferably the intervening peptide sequence is between 2 and 8 amino acids long. In one example the intervening peptide sequence is 2 amino acid long and may for example comprise the sequence LC. In another example the intervening peptide sequence is 4 amino acids long and may for example comprise the sequence LCCM. In another example the intervening peptide sequence may be 5 amino acids long and may for example comprise the sequence LSLWM. In another example the intervening peptide sequence may be 7 amino acids long and may for example comprise the sequence AGLSLWM. In a further example the intervening peptide sequence may be 8 amino acids long and may for example comprise the sequence MAGLSLWM.

For example the TMCT of the modified S-protein may comprise the following sequence or a sequence that has 90-100%, or any amount therebetween sequence identity, or sequence similarity to:

(SEQ ID NO: 64)
WYIWLGFIAGLIAIVMVTIM - (X)n - CSNGSXXCXICI,
(SEQ ID NO: 134)
WYVWLGFIAGLIAIVMVTIL - (X)n - CSNGSXXCXICI,
or
(SEQ ID NO: 135)
WYIWLGFIAGLVALALCVFFIL - (X)n - CSNGSXXCXICI,
(SEQ ID NO: 172)
WYVWLLICLAGVAMLVLLFFI - (X)n - CSNGSXXCXICI,
(SEQ ID NO: 173)
WWVWLCISVVLIFVVSMLLL - (X)n - CSNGSXXCXICI,

wherein (X)ₙ is the intervening peptide sequence, wherein the intervening peptide sequence may have a length from 0 to n amino acid residues, wherein n may be any length from 0-10, for example 0, 1, 2, 3, 4, 5, 6, 7, 8, 9 or 10 amino acids and wherein X may comprise any amino acid, for example X may be A, C, G, F, L, S, M, W or conserved substitution of A, C, G, F, L, S, M, W, or a combination thereof.

Non-limiting intervening peptide sequences (X)ₙ may include the following:
(X)₀, the intervening peptide sequence is absent;
(X)₁, comprising for example the sequence L or C, or conserved substitution of L or C;
(X)₂, comprising for example the sequence LC, or conserved substitution of LC;
(X)₃, comprising for example the sequence LCC, or conserved substitution of LCC;
(X)₄, comprising for example the sequence LCCM, or conserved substitutions of LCCM, SLWM or a conserved substitution of SLWM, SFWM or a conserved substitution of SFWM;
(X)₅, comprising for example the sequence LSLWM, or conserved substitutions of LSLWM, LSLWM or a conserved substitution of LSLWM, LSFWM or a conserved substitution of LSFWM;
(X)₆, comprising for example the sequence GLSLWM, or conserved substitutions of GLSLWM;
(X)₇, comprising for example the sequence AGLSLWM, or conserved substitutions of AGLSLWM, or
(X)₈, comprising for example the sequence MAGLSLWM, or conserved substitutions of MAGLSLWM.

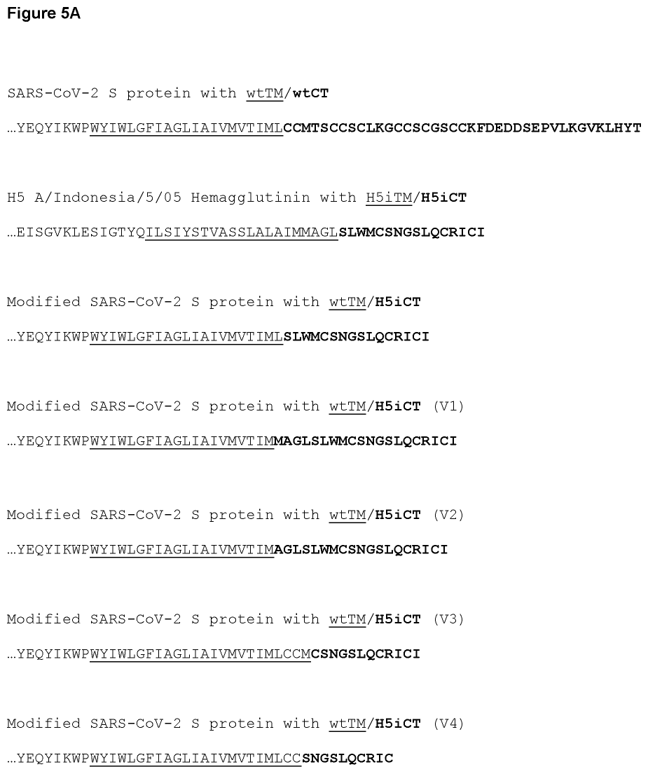

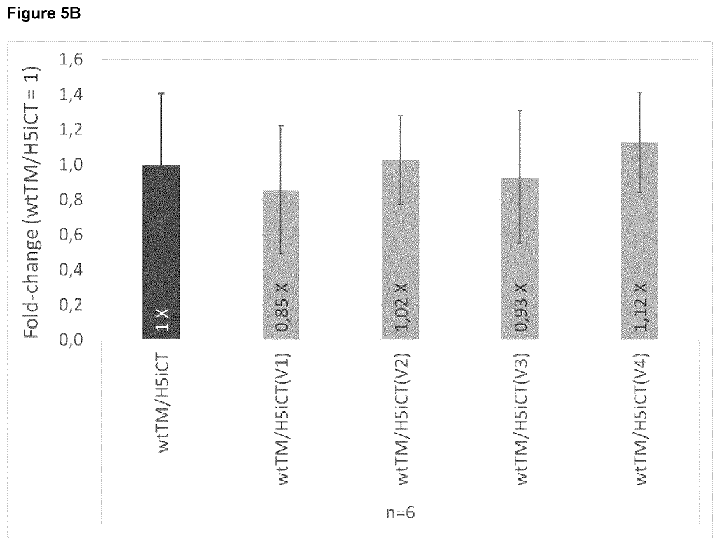

FIG. 5B shows the fold change of protein accumulation of modified S protein with alternative versions of the C-terminal region with variable margin (intervening peptide sequence) between the SARS-COV-2 transmembrane (TM) domain and H5 A/Indonesia/5/05 HA cytosolic tail (CT) domain, wtTM/H5iCT V1 (SEQ ID NO: 19, product of construct 8980), wtTM/H5iCT V2 (SEQ ID NO: 37, product of construct 8981), wtTM/H5iCT V3 (SEQ ID NO: 38, product of construct #8982) and wtTM/H5iCT V4 (SEQ ID NO: 39, product of construct 8983), when expressed in plants, compared to the protein accumulation of a reference modified S protein wherein the cytoplasmic tail of Coronavirus S-protein has been replaced with the Coronavirus S-protein of H5 A/Indonesia/5/05 HA wtTM/H5iCT (SEQ ID NO: 18, product of construct 8671). All tested modified S proteins showed protein accumulation, with no statistically significant differences between the alternative versions and the wtTM/H5iCT reference control.

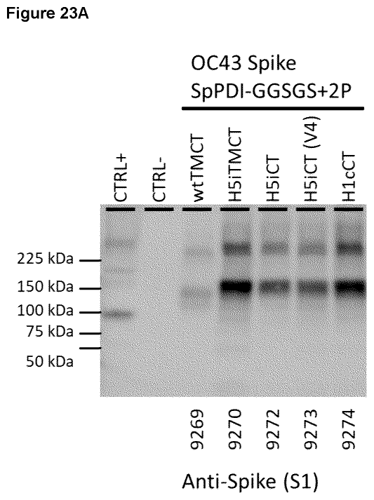

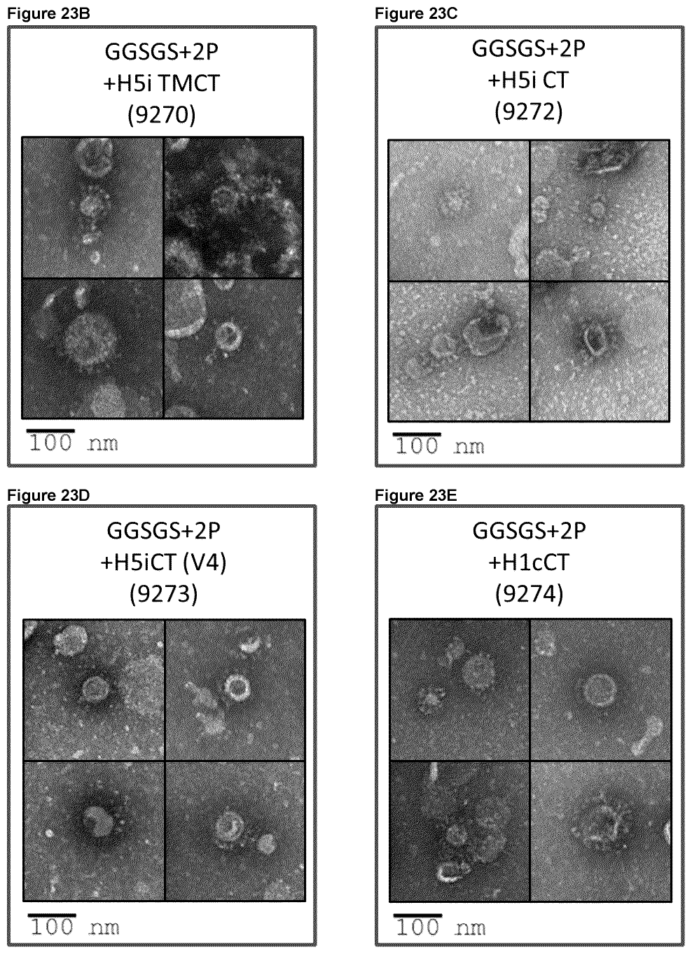

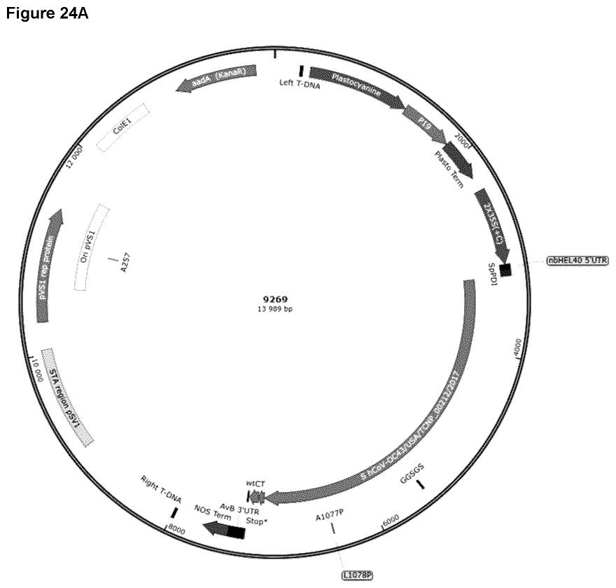

Similarly, a modified S protein comprising a SARS-CoV-1 S protein with a wtTM/H5iCT V4 version of the TMCT (FIG. 16A) or a MERS S protein with a wtTM/H5iCT V4 version of the TMCT (FIG. 19A), when expressed in plants, showed increased protein accumulation compared to protein accumulation of the wild type S proteins (wtTMCT) or S proteins wherein the TMCT has been replaced with the TMCT of H5 A/Indonesia/5/05 HA (H5iTMCT). Furthermore, a OC43 CoV S-protein with a wtTM/H5iCT V4 version of the TMCT when expressed in plants, showed increased protein accumulation compared to protein accumulation of the OC43 CoV S-protein with wild type TMCT (wtTMCT) (FIG. 23 A).

Accordingly, the modified S protein may comprise a TM and CT domain (TM/CT), wherein the CT or a portion of the CT is fused to the C-terminal end of the TM or portion of the TM via a intervening peptide sequence, wherein the intervening peptide sequence comprises the sequence Xₙ.

Furthermore, the modified S protein may comprise a TM and CT domain (TM/CT) comprising a sequence having about 70, 75, 80, 85, 87, 90, 91, 92, 93 94, 95, 96, 97, 98, 99, 100% or any amount therebetween sequence identity, or sequence similarity, with the sequence of SEQ ID NO: 18, 19, 37, 38, 39, 64, 126, 127, 128, 129, 130, 131, 118, 119, 120, 123, 124, 125, 134, 135, 164, 165, 166, 169, 170, 171, 172 or 173.

The modified S protein may comprise a CT or portion of the CT comprising a sequence having about 70, 75, 80, 85, 87, 90, 91, 92, 93 94, 95, 96, 97, 98, 99, 100% or any amount therebetween sequence identity, or sequence similarity, with amino acids 22-37 of SEQ ID NO:18, amino acids 21-40 of SEQ ID NO: 19, amino acids 21-39 of SEQ ID NO: 37, amino acids 25-36 of SEQ ID NO: 38 or amino acids 24-34 of SEQ ID NO: 39, amino acids 22-37 of SEQ ID NO:126, amino acids 22-36 of SEQ ID NO:127, amino acids 22-37 of SEQ ID NO:128, amino acids 22-37 of SEQ ID NO:129, amino acids 22-37 of SEQ ID NO:130, or amino acids 22-37 of SEQ ID NO:131.

The modified S protein may comprise a TM or portion of the TM comprising a sequence having about 70, 75, 80, 85, 87, 90, 91, 92, 93 94, 95, 96, 97, 98, 99, 100% or any amount therebetween sequence identity, or sequence similarity, with amino acids 1-20 of SEQ ID NO:18, amino acids 1-20 of SEQ ID NO: 19, amino acids 1-20 of SEQ ID NO: 37, amino acids 1-24 of SEQ ID NO: 38, amino acids 1-23 of SEQ ID NO: 39, amino acids 1-21 of SEQ ID NO: 118, amino acids 1-23 of SEQ ID NO: 119, amino acids 1-22 of SEQ ID NO: 123, amino acids 1-24 of SEQ ID NO: 124, amino acids 1-21 of SEQ ID NO: 164, amino acids 1-23 of SEQ ID NO: 165, amino acids 1-20 of SEQ ID NO: 169, or amino acids 1-22 of SEQ ID NO: 170. Furthermore, the modified S protein as described herewith may comprise a TM or portion of TM that comprises from 80% to 100% identity with the sequence of SEQ ID NO: 132 or 133.

Furthermore, the modified the S-protein may comprise from 70% to 100% sequence identity, or sequence similarity, with the sequence of SEQ ID NO: 5, 59, 60, 61, 62, 95, 96, 97, 108, 109 or 110, for example the modified S protein may comprise a sequence having about 70, 75, 80, 85, 87, 90, 91, 92, 93, 94, 95, 96, 97, 98, 99, 100% or any amount therebetween sequence identity, or sequence similarity, with the sequence of SEQ ID NO: 5, 59, 60, 61, 62, 95, 96, 97, 108, 109 or 110.

The cytoplasmic tail domain (CT) or portion of the CT of a viral structural protein such as for example a Coronavirus S protein, may be replaced with the CT or portion of the CT from an influenza hemagglutinin (HA) as described below, the resulting protein is referred to as modified viral structural protein. Accordingly, a coronavirus S protein wherein the native CT or portion of the native CT has been replaced with the CT or portion of the CT from HA may be referred to as modified coronavirus S-protein or modified S-protein. As further described above the HA CT or portion of the HA CT may either be directly fused to the N-terminal end of the Coronavirus TM domain or may be fused to the N-terminal end of the Coronavirus TM or portion of the TM via a intervening peptide sequence. Therefore, the HA CT or a portion of a HA CT may be fused to the C-terminal end of the S-protein TM or portion of the S-protein TM via an intervening peptide sequence.

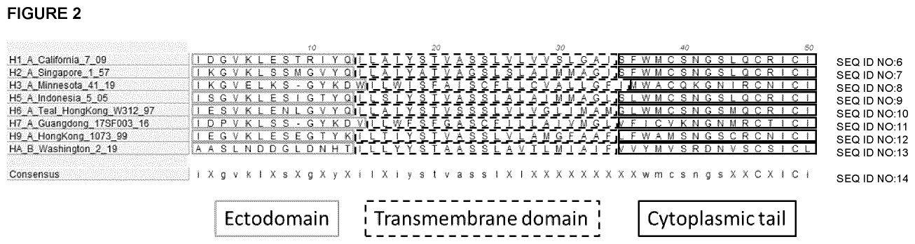

Influenza “hemagglutinin” or “HA” is a homotrimeric membrane type I glycoprotein, generally comprising a signal peptide, an HA1 domain, and an HA2 domain comprising a membrane-spanning anchor site at the C-terminus and a small cytoplasmic tail (see for example FIG. 1C and FIG. 2). The amino acid sequences of HA from various influenza strains are well known within the art. Furthermore, amino acid sequences and nucleotide sequences encoding HA are well known and are available-see, for example, the BioDefence Public Health base (Influenza Virus; see URL: biohealthbase.org) or National Center for Biotechnology Information (see URL: ncbi.nlm.nih.gov), both of which are incorporated herein by reference. Exemplary amino acid sequences of HA cytoplasmic tail domains from different influenza strains are shown in FIG. 2.

While different references and groups assign different length to the CT of HA, it has been shown that the N-terminal sequence of the CT is conserved among HA from different influenza subtypes and strains and that at least five residues have sequence identity for at least 10 of 13 HA subtypes (Simpson and Lamb 1992, Journal of Virology, 790-803). FIG. 2 shows an alignment of amino acid sequences from exemplary influenza strains and conserved sequences in the N-terminal part of the HA protein. The consensus sequence of influenza cytoplasmic tail (CT) domain is:

(SEQ ID NO: 15)
XXWMCSNGSXXCXICI (see also FIG. 2, C-terminal end
of SEQ ID NO: 14)

CT sequences that correspond to the HA cytoplasmic tail domain consensus sequence may be fused to the C-terminal end of the TM of Coronavirus S protein either directly or via an intervening peptide sequence (linker sequence) as discussed above.

Furthermore, amino acid residues located in N-terminal or C-terminal from the native influenza HA TM/CT boundary may also be included in the CT sequence that is fused either directly or via an intervening peptide sequence to the TM or a portion of the TM of the modified Coronavirus S protein.

Therefore the sequence of the CT or a portion of the CT may for example start at an amino acid residue that corresponds to any one of amino acids 30-40 of SEQ ID NO: 14. Accordingly, the N-terminal end of the CT sequence may be an amino acid that corresponds to any one of amino acids 30-40 of SEQ ID NOs: 6, 7, 8, 9, 10, 11, 12, 13 or 14. In one example the CT sequence may start at an amino acid residue that corresponds to amino acid 30 in SEQ ID NOs: 6, 7, 8, 9, 10, 11, 12, 13 or 14. In another example, the CT sequence may start at an amino acid residue that corresponds to amino acid 31 of SEQ ID NOs: 6, 7, 8, 9, 10, 11, 12, 13 or 14. In a further example, the CT sequence may start at an amino acid residue that corresponds to amino acid 32 of SEQ ID NOs: 6, 7, 8, 9, 10, 11, 12, 13 or 14. In another example, the CT sequence may start at an amino acid residue that corresponds to amino acid 33 of SEQ ID NOs: 6, 7, 8, 9, 10, 11, 12, 13 or 14. In a further example, the CT sequence may start at an amino acid residue that corresponds to amino acid 34 of SEQ ID NOs: 6, 7, 8, 9, 10, 11, 12, 13 or 14. In another example, the CT sequence may start at an amino acid residue that corresponds to amino acid 35 of SEQ ID NOs: 6, 7, 8, 9, 10, 11, 12, 13 or 14. In a further example, the CT sequence may start at an amino acid residue that corresponds to amino acid 36 of SEQ ID NOs: 6-13 or 14. In another example, the CT sequence may start at an amino acid residue that corresponds to amino acid 37 of SEQ ID NOs: 6, 7, 8, 9, 10, 11, 12, 13 or 14. In a further example, the CT sequence may start at an amino acid residue that corresponds to amino acid 38 of SEQ ID NOs: 6-13 or 14. In another example, the CT sequence may start at an amino acid residue that corresponds to amino acid 39 of SEQ ID NOs: 6, 7, 8, 9, 10, 11, 12, 13 or 14. In a further example, the CT sequence may start at an amino acid residue that corresponds to amino acid 40 of SEQ ID NOs: 6, 7, 8, 9, 10, 11, 12, 13 or 14.

The cytoplasmic tail (CT) or portion of the CT of the modified S protein may be derived from a CT or portion of the CT of hemagglutinin (HA) of any one influenza type, subtype or strain. For example the CT may be derived from an HA from influenza type A or influenza type B. For example the CT may be derived from an HA of influenza subtype H1, H2, H3, H4, H5, H6, H7, H8, H9, H10, H11, H12, H13, H14, H15, or H16. The CT may for example be derived from a HA of subtype H1, H2, H3, H5, H6, H7 or H9. Furthermore, the CT or portion of the CT may be derived from an HA of influenza type B. The type B influenza may be from the lineage B/Yamagata or B/Victoria.

For example, the CT or portion of the CT of the modified S protein may be derived from a CT of hemagglutinin (HA) influenza H1, H3, H5, H6, H7, H9 or B strain. Non limiting examples of influenza stains from which the HA CT might be derived are influenza H1 California/7/2009, H2 A/Singapore/1/1957, H3 A/Minnesota/41/2019, H5 A/Indonesia/5/05, H6 A/Teal/Hong Kong/W312/97, H7 A/Guangdong/17SF003/2016, H9 A/Hong Kong/1073/99 or B/Washington/02/2019. Non limiting examples of amino acid sequences of the HA CT are shown in FIG. 2.

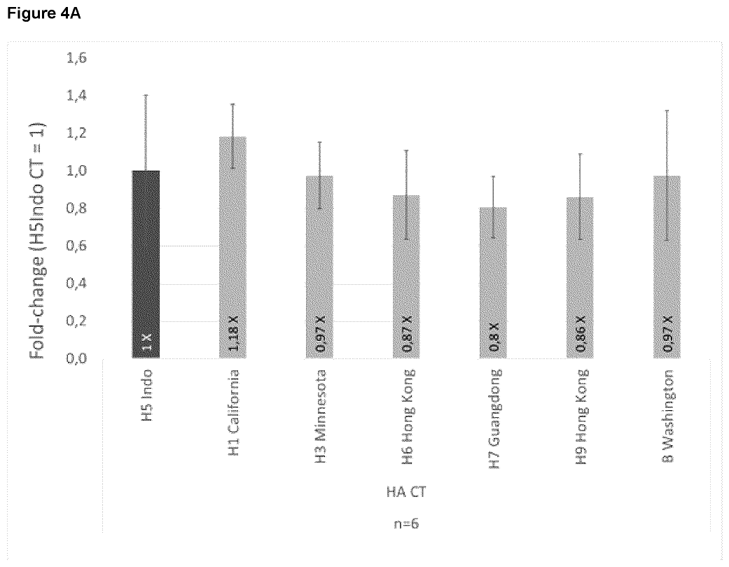

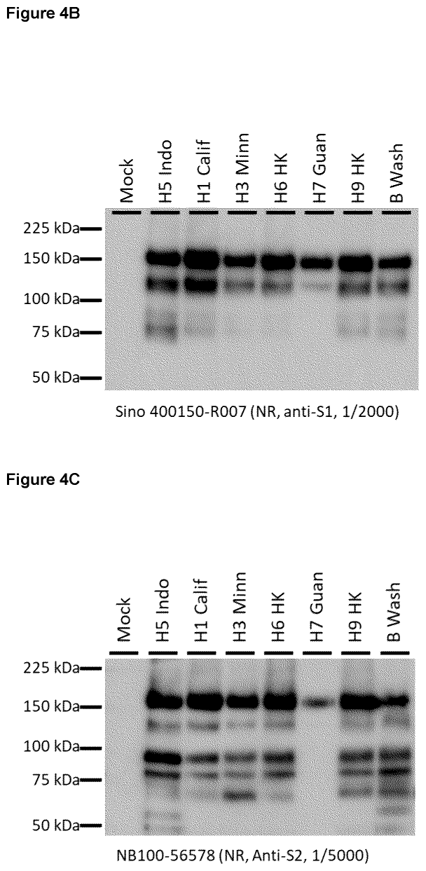

As shown in FIG. 4A, when the native cytoplasmic tail (CT) of SARS-CoV-2 S protein was replaced with the CT from influenza HA H1 California/7/2009 (H1 Calif), H3 A/Minnesota/41/2019 (H3 Minn), H6 A/Teal/Hong Kong/W312/97 (H6 HK), H7 A/Guangdong/17SF003/2016 (H7 Guan), H9 A/Hong Kong/1073/99 (H9 HK) or B/Washington/02/2019 (B Wash), similar fold change in protein accumulation were observed for these modified SARS-CoV-2 S protein, when compared to SARS-CoV-2 S with a CT from H5 A/Indonesia/5/05 (H5 Indo). Western blot analysis confirmed these observations (see FIGS. 4B and 4C).

Similar results were obtained, when the native cytoplasmic tail (CT) of SARS-CoV-1 S protein, the native CT of MERS S protein, or the native CT of OC43 CoV S protein was replaced with the CT from influenza HA H1 California/7/2009 (H1cCT) (see FIGS. 16A, 19A, and 23A).

Accordingly, the cytoplasmic tail domain (CT) or portion of the CT may have about 70, 75, 80, 85, 86, 87, 88, 89, 90, 91, 92, 93 94, 95, 96, 97, 98, 99, 100% or any amount therebetween sequence identity, or sequence similarity, with the sequence of SEQ ID NO: 15, or with amino acids 30-50 of SEQ ID NO 6, 7, 8, 9, 10, 12, 13, 14, or with amino acids 31-50 of SEQ ID NO 6, 7, 8, 9, 10, 12, 13, 14, or with amino acids 32-50 of SEQ ID NO 6, 7, 8, 9, 10, 12, 13, 14, or with amino acids 33-50 of SEQ ID NO 6, 7, 8, 9, 10, 12, 13, 14, or with amino acids 34-50 of SEQ ID NO 6, 7, 8, 9, 10, 12, 13, 14, or with amino acids 35-50 of SEQ ID NO 6, 7, 8, 9, 10, 12, 13, 14, or with amino acids 36-50 of SEQ ID NO 6, 7, 8, 9, 10, 12, 13, 14, or with amino acids 37-50 of SEQ ID NO 6, 7, 8, 9, 10, 12, 13, 14, or with amino acids 38-50 of SEQ ID NO 6, 7, 8, 9, 10, 12, 13, 14, or with amino acids 39-50 of SEQ ID NO 6, 7, 8, 9, 10, 12, 13, 14, or with amino acids 40-50 of SEQ ID NO 6, 7, 8, 9, 10, 12, 13, 14, or with amino acids 31-49 of SEQ ID NO 11, or with amino acids 32-49 of SEQ ID NO 11, or with amino acids 33-49 of SEQ ID NO 11, or with amino acids 34-49 of SEQ ID NO 11, or with amino acids 35-49 of SEQ ID NO 11, or with amino acids 36-49 of SEQ ID NO 11, or with amino acids 37-49 of SEQ ID NO 11, or with amino acids 38-49 of SEQ ID NO 11, or with amino acids 39-49 of SEQ ID NO 11, or with amino acids 548-568 of SEQ ID NO:3, or with amino acids 549-568 of SEQ ID NO:3, or with amino acids 550-568 of SEQ ID NO:3, or with amino acids 551-568 of SEQ ID NO:3, or with amino acids 552-568 of SEQ ID NO:3, or with amino acids 553-568 of SEQ ID NO:3, or with amino acids 554-568 of SEQ ID NO:3, or with amino acids 555-568 of SEQ ID NO:3, or with amino acids 556-568 of SEQ ID NO:3, or with amino acids 557-568 of SEQ ID NO:3, or with amino acids 558-568 of SEQ ID NO:3.

Furthermore, the modified S-protein may comprise from 70% to 100% sequence identity, or sequence similarity, with the sequence of SEQ ID NO: 5, 53, 54, 55, 56, 57 or 58, for example the modified S protein may comprise a sequence having about 70, 75, 80, 85, 87, 90, 91, 92, 93, 94, 95, 96, 97, 98, 99, 100% or any amount therebetween sequence identity, or sequence similarity, with the sequence of SEQ ID NO: 5, 53, 54, 55, 56, 57 or 58 or with amino acids 25-1259 of SEQ ID NO: 53, amino acids 25-1259 of SEQ ID NO: 54, amino acids 25-1259 of SEQ ID NO: 55, amino acids 25-1259 of SEQ ID NO: 56, amino acids 25-1259 of SEQ ID NO: 57 or amino acids 25-1259 of SEQ ID NO: 58.

In further embodiments, the modified S protein ectodomain and/or transmembrane domain may be obtained from a coronavirus S protein other than SARS-CoV-2 S protein, for example from SARS-CoV-1 S protein, MERS-CoV S protein, OC43-CoV S protein, 229E-CoV S protein and the like.

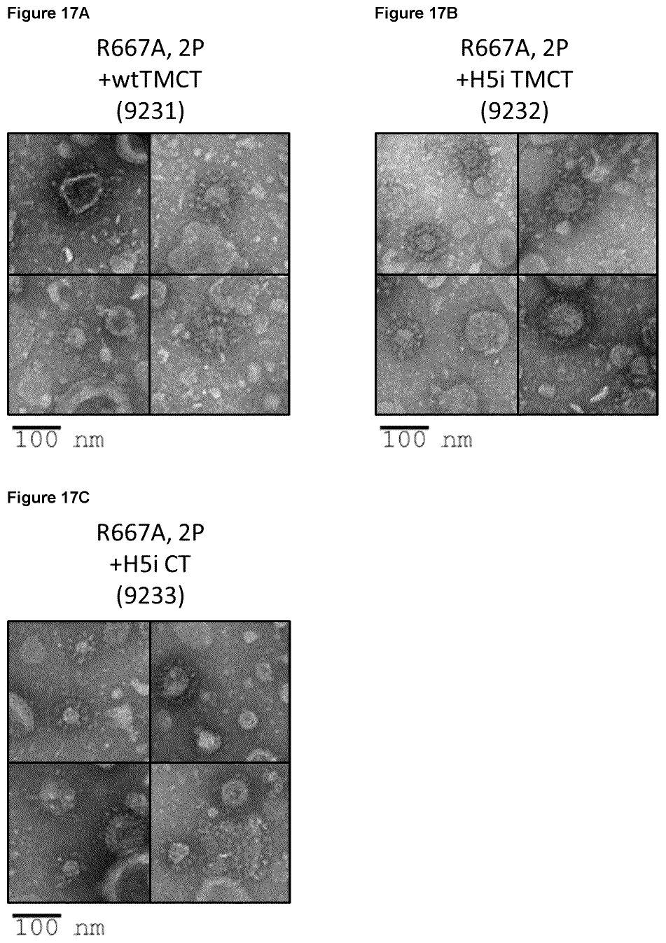

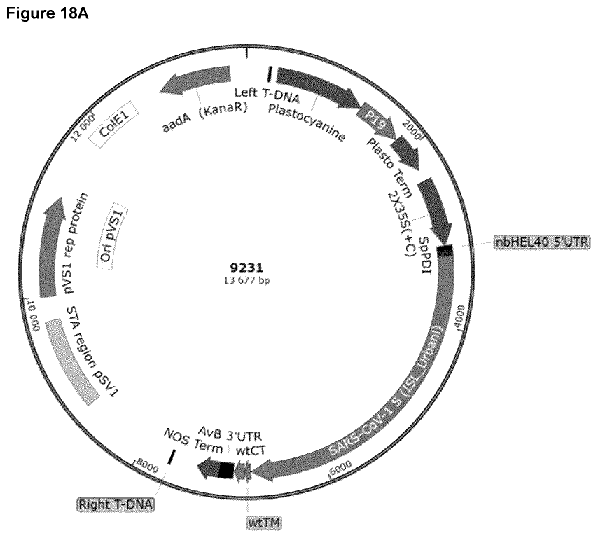

As shown in FIG. 16A, higher protein accumulation was observed for modified SARS-CoV-1 S protein with a CT from influenza H5 HA (H5iCT), a CT from influenza H1 HA (H1cCT) or a CT from influenza H5 HA with a variable margin between the SARS-CoV-1 TM and the H5 HA CT (“H5iCT(V4)), when compared to protein accumulation of S protein with a wild-type TMCT (wt TMCT) or a modified S protein with the TMCT of influenza H5 HA (H5i TMCT) from crude plant extract. The modified S-proteins assembled into high molecular weight structures (see FIGS. 16B-16D), which were confirmed to be VLPs (see FIGS. 17A-17C). Although the amount of protein accumulation for SARS-CoV-1 protein with native TMCT was below detection limits of the Western Blot analysis when presented on the same gel with SARS-CoV-1 S-proteins with modified TMCT and/or CT (see FIG. 16A), signals could be detected by Western Blot analysis when only SARS-CoV-1 protein with native TMCT was present on a gel (see FIG. 16B) and VLPs were observed by electron microscopy (see FIG. 17B).

Similar results were obtained for modified MERS-CoV S protein (see FIG. 19A). Higher protein accumulation was observed for modified MERS-CoV S protein with a CT from influenza H5 HA (H5iCT), a CT from influenza H1 HA (H1cCT) or a CT from influenza H5 HA with a variable margin between the MERS-CoV TM and the H5 HA CT (“H5iCT(V4)), when compared to protein accumulation of S protein with a wild-type TMCT (wt TMCT) or a modified S protein with the TMCT of influenza H5 HA (H5i TMCT) (see protein band at approx. 175 kDa). The highest accumulation was observed for the modified MERS-CoV with an influenza H1 HA CT (H1cCT). The smaller band observed at approx. 100 kDa is most likely a proteolytic cleavage fragment of the S-protein. Without wishing to be bound by theory, it is believed that the replacement of the native CT with an influenza HA CT stabilizes the MERS S-protein and reduces cleavage of the S-protein.

As further shown in FIG. 23A, low protein yields were observed in plants expressing OC43 CoV S-protein with a native OC43 CoV S-protein TMCT. However, when the native OC43 CoV S-protein TMCT was replaced with a TMCT from influenza H5 HA (H5iTMCT), a CT from influenza H5 HA (H5iCT), a CT from influenza H1 HA (H1cCT) or a CT from influenza H5 HA with a variable margin between the OC43-CoV TM and the H5 HA CT (“H5iCT(V4)) higher protein accumulations were observed compared to the OC43 CoV S-protein with native TMCT (see bands at about 150 kDa. The larger band shown in the gel is believed to be a protein trimer). Similar results were observed with modified 229E-CoV S-protein (data not shown).

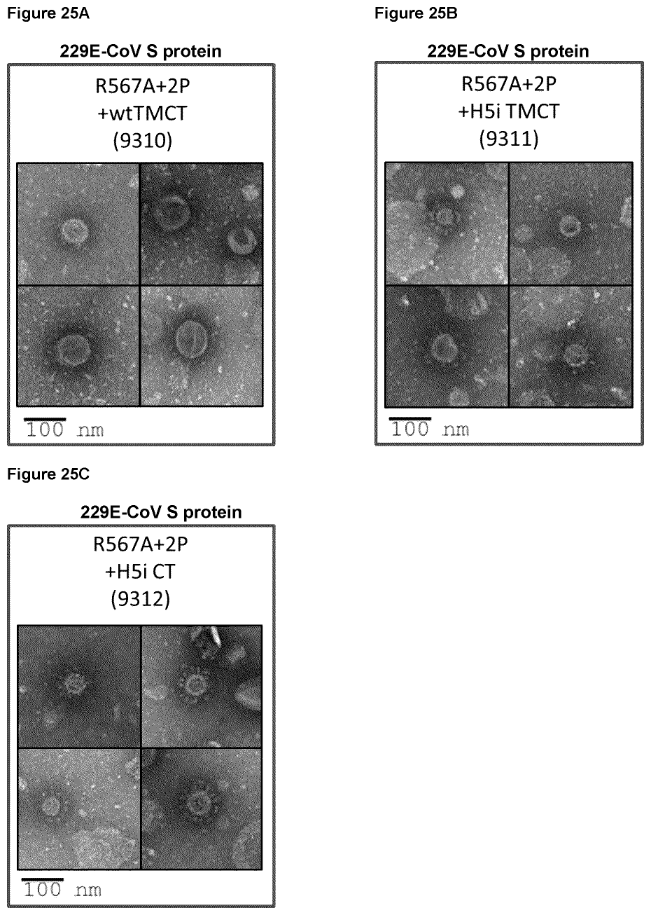

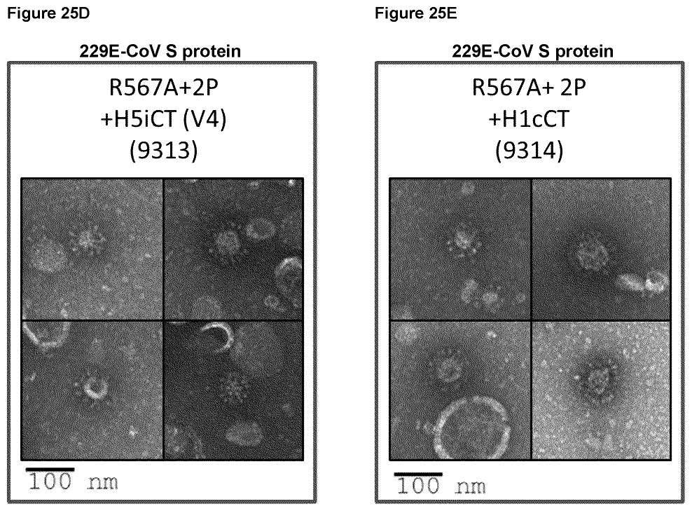

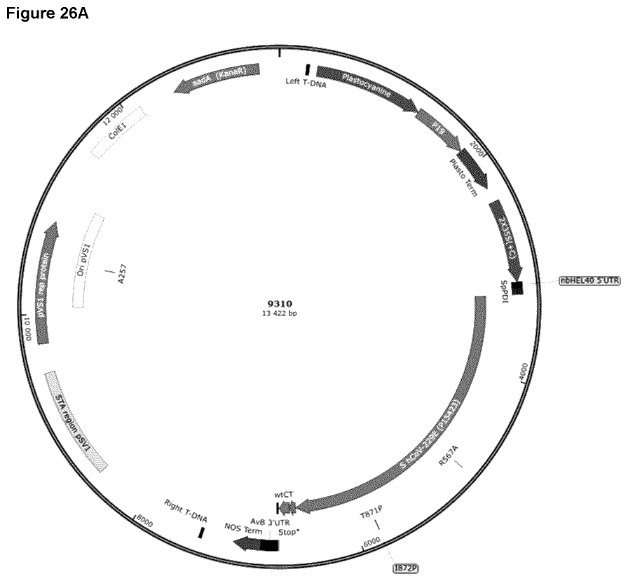

Furthermore, MERS-CoV S-protein, OC43-CoV S-protein, and 229E-CoV S-protein with a TMCT from influenza H5 HA (H5iTMCT), a CT from influenza H5 HA (H5iCT), or a CT from influenza H1 HA were observed to form VLPs as shown in FIGS. 19B-19F, 23B-23E, and 25A-25E.

The present disclosure therefore provides a “modified viral structural protein”, a “viral structural fusion protein” or a “chimeric viral structural protein”, wherein the ectodomain and the transmembrane domain (TM) of the viral structural protein or a portion of the TM are derived from a Coronavirus and the cytosolic tail (CT) or a portion of the CT is derived from an influenza protein. For example, the ectodomain and the transmembrane domain may be derived from a Coronavirus Spike (S) protein and the cytosolic tail (CT) or a portion of the CT may be derived from influenza HA protein. Modified S protein may comprise, in series i) an ectodomain derived from a coronavirus S-protein (comprising the S1 subunit and the FP, HR1 and HR2 domains of the S2 subunit), ii) a Coronavirus transmembrane domain (TM) or a portion of a Coronavirus TM and iii) an influenza HA cytoplasmic tail domain (CT) or a portion of a HA CT. Therefore, in the modified S protein, the CT or portion of the CT is heterologous to the TM and the ectodomain. Similarly, the TM (and the ectodomain) of the modified S protein are heterologous to the CT. The ectodomain and the transmembrane domain (TM) may be derived from the same Coronavirus (i.e. the ectodomain and the TM may be homologous to each other) or the ectodomain may be derived from a first Coronavirus and the TM may be derived from a second Coronavirus (i.e. the ectodomain and the TM are heterologous to each other).

By “chimeric protein”, or “chimeric polypeptide”, also referred to as a “fusion protein”, it is meant a protein or polypeptide that comprises amino acid sequences from two or more than two sources, for example but not limited to an ectodomain and a transmembrane domain derived from a first viral structural protein for example derived from Coronavirus S protein and a cytoplasmic tail (CT) derived from a second viral structural protein for example a CT from influenza HA, that are fused as a single polypeptide.

The modified coronavirus S-protein may comprise a transmembrane and cytosolic tail domain (TMCT), wherein the TMCT is a chimeric TMCT. The chimeric TMCT may comprise a transmembrane domain (TM), wherein the TM or a portion of the TM is derived from a coronavirus S-protein and a cytosolic tail (CT), wherein the CT or a portion of the CT is derived from an influenza hemagglutinin (HA) protein. Furthermore the chimeric TMCT may comprise a native coronavirus S-protein TM, a chimeric coronavirus S-protein/influenza HA TM, a native influenza HA CT, a chimeric influenza HA/coronavirus S-protein CT or a combination thereof. For example, the modified coronavirus S-protein may comprise a chimeric TMCT with a native influenza HA CT and a chimeric TM, wherein the chimeric TM comprises a N-terminal sequence which is derived from the TM of the coronavirus S-protein and a C-terminal sequence which is derived from the TM of influenza HA protein. In another example the modified coronavirus S-protein may comprise a chimeric TMCT with a native coronavirus S-protein TM and a chimeric CT, wherein the chimeric CT comprises a N-terminal sequence derived from the coronavirus S-protein and a C-terminal sequence derived from the influenza HA protein. In a further example, the modified coronavirus S-protein may comprise a chimeric TMCT with a chimeric TM, wherein the chimeric TM comprises a N-terminal sequence which is derived from the TM of the coronavirus S-protein and a C-terminal sequence which is derived from the TM of influenza HA protein and a chimeric CT, wherein the chimeric CT comprises a N-terminal sequence derived from the coronavirus S-protein and a C-terminal sequence derived from the influenza HA protein.

When referring to a modified S-protein or modified coronavirus spike (S)-protein in the present disclosure, it is meant a modified coronavirus spike (S)-protein comprising a transmembrane domain (TM) or portion of a S-protein TM, and a cytosolic tail (CT) or a portion of a CT, wherein the CT is derived from an influenza hemagglutinin (HA) protein and wherein the TM is heterologous to the CT.

The modified the S-protein may comprise from 70% to 100% sequence identity, or sequence similarity, with the sequence of SEQ ID NO: 5, 21, 30, 47, 48, 49, 50, 51, 52, 53, 54, 55, 56, 57, 58, 59, 60, 61, 62, 95, 96, 97, 108, 109, 110, 144, 145, 146, 155, 156 or 157, for example the modified S protein may comprise a sequence having about 70, 75, 80, 85, 87, 90, 91, 92, 93, 94, 95, 96, 97, 98, 99, 100% or any amount therebetween sequence identity, or sequence similarity, with the sequence of SEQ ID NO: 5, 21, 30, 47, 48, 49, 50, 51, 52, 53, 54, 55, 56, 57, 58, 59, 60, 61, 62, 95, 96, 97, 108, 109, 110, 144, 145, 146, 155, 156 or 157, or with amino acids 25-1259 of SEQ ID NO: 47, amino acids 25-1259 of SEQ ID NO: 48, amino acids 25-1259 of SEQ ID NO: 49, amino acids 25-1259 of SEQ ID NO: 50, amino acids 25-1259 of SEQ ID NO: 51, amino acids 25-1259 of SEQ ID NO: 52, amino acids 25-1259 of SEQ ID NO: 53, amino acids 25-1259 of SEQ ID NO: 54, amino acids 25-1259 of SEQ ID NO: 55, amino acids 25-1259 of SEQ ID NO: 56, amino acids 25-1259 of SEQ ID NO: 57, amino acids 25-1259 of SEQ ID NO: 58, amino acids 25-1262 of SEQ ID NO: 59, amino acids 25-1261 of SEQ ID NO: 60, amino acids 25-1258 of SEQ ID NO: 61, amino acids 25-1256 of SEQ ID NO: 62, amino acids 25-1243 of SEQ ID NO: 95, amino acids 25-1240 of SEQ ID NO: 96, amino acids 25-1243 of SEQ ID NO: 97, amino acids 25-1341 of SEQ ID NO: 108, amino acids 25-1338 of SEQ ID NO: 109, amino acids 25-1341 of SEQ ID NO: 110, amino acids 25-1351 of SEQ ID NO: 144, amino acids 25-1348 of SEQ ID NO: 145, amino acids 25-1351 of SEQ ID NO: 146, amino acids 25-1159 of SEQ ID NO: 155, amino acids 25-1156 of SEQ ID NO: 156, or amino acids 25-1159 of SEQ ID NO: 157.

The modified S-protein may further be produced or synthesized as modified S-protein precursor (also referred to as precursor S-protein), wherein the S-protein precursor comprises the modified S-protein and a signal peptide, wherein the signal peptide is native to Coronavirus (i.e. homologues to the ectodomain) or the signal peptide might be non-native or heterologous to the ectodomain. In a non-limiting example, the native signal peptide may be replaced with the signal peptide from protein disulfide isomerase (PDI).

The modified S-protein precursor may comprise a signal peptide that is non-native or heterologous to the ectodomain. The non-native signal peptide may replace the entire native signal peptide or may replace a portion of the native signal peptide of the Coronavirus S protein. Furthermore, the non-native or heterologous signal peptide may be directly fused to the N-terminus of the modified S protein or the non-native or heterologous signal peptide may be fused to the N-terminus of the modified S protein with an intervening peptide sequence.

A signal peptide (also referred to as signal sequence, targeting signal, localization signal, localization sequence, transit peptide, leader sequence or leader peptide) is a short peptide present at the N-terminus of the majority of newly synthesized proteins that are destined toward the secretory pathway. The signal peptide is responsible for targeting proteins to the endomembrane system, including the endoplasmic reticulum and the Golgi apparatus, where it is co-translationally removed by a signal peptidase located within the ER lumen and the mature proteins are generated. Since experimental methods for identification of targeting sequences are time-consuming and laborious, different computational approaches predicting targeting signals were developed, and are well known within the art. Signal peptides generally have low sequence similarity, but share some characteristic features. For predicting the signal sequence and its cleavage site, many prediction methods have been developed which take these characteristic features into account, such for example SignalP (Bendtsen et al., J Mol Biol. 2004 Jul. 16; 340(4):783-95.; Petersen et al., Nature Methods volume 8, pages 785-786(2011), Signal-CF (Chou and Shen, Biochem Biophys Res Commun. 2007 Jun. 8; 357(3):633-40), and Signal-BLAST (Frank and Sippl, Bioinformatics, 2008 Oct. 1; 24(19):2172-6), which are herewith incorporated by reference.

Using the SignalP prediction program, a signal peptide cleavage site for the SARS-CoV-2 S protein is predicted between position 15 and 16 of the sequence corresponding to the sequence of SEQ ID NO:1. However, a signal peptide cleavage site for the SARS-CoV-2 S protein may be predicted or occur between other consecutive positions of the sequence corresponding to the sequence of SEQ ID NO:1. For example, a signal peptide cleavage site for the SARS-CoV-2 S protein may also be predicted or may occur between position 13 and 14 of the sequence corresponding to the sequence of SEQ ID NO:1.

The N-terminal region of the native SARS-CoV-2 S protein (including the native signal peptide sequence) is shown below:

(SEQ ID NO: 63)
MFVFLVLLPLV [symbol] QLPPAYTNS

A predicted signal peptide sequence (SP) is underlined. The sequence shaded in grey corresponds to the sequence depicted in Table 2. The first amino acid residue of the mature SARS-CoV-2 S protein may be Valine (V) with its position designated as 1 (+1), which corresponds to V16 of the precursor S protein (native SARS-CoV-2 S protein with the native signal peptide). The first amino acid residue of the mature SARS-CoV-2 S protein may be at other residues of SEQ ID NO:1 or SEQ ID NO: 63 as indicated in Table 2. For example, the first amino acid residue of the mature SARS-CoV-2 S protein may be Glutamine (Q) with its position designated as 14 (-2).

TABLE 2
Portion of the SARS-CoV-2 S-protein sequence surrounding a signal
peptide (SP) cleavage site. Numbering of residues is either
from the N-terminus that includes the native signal peptide
(# with SP) or from a predicted cleavage site at position
V1 (# without SP), which is equivalent to V16 in the
sequence that includes the signal peptide (precursor S protein).

| | S | S | Q | C | V | N | L | T | T | R | T |
|---|---|---|---|---|---|---|---|---|---|---|---|
| #_with_SP | 12 | 13 | 14 | 15 | 16 | 17 | 18 | 19 | 20 | 21 | 22 |
| #_without_SP* | −4 | −3 | −2 | −1 | 1 | 2 | 3 | 4 | 5 | 6 | 7 |

*mature protein

Signal peptides or peptide sequences for directing localization of an expressed protein or polypeptide to the apoplast include, but are not limited to, a native (with respect to the protein) signal or leader sequence, or a heterologous signal sequence, for example but not limited to, a rice amylase signal peptide (McCormick 1999, Proc Natl Acad Sci USA 96:703-708) or a protein disulfide isomerase signal peptide (PDI). Therefore, as described herein, the modified S protein may be produced as precursor protein comprising a modified S-protein and a heterologous amino acid signal peptide sequence. For example, the modified S protein precursor may comprise the signal peptide from Protein disulphide isomerase (PDI SP; nucleotides 32-103 of Accession No. Z11499).

The present disclosure therefore also provides for a modified S protein precursor comprising a modified S-protein and a native, or a non-native signal peptide, and nucleic acids encoding such protein.

The modified viral structural protein may be a modified S protein, wherein the modified S protein is a monomeric or single chain modified S protein. The monomeric or single chain modified S protein may include an S1 domain (subunit) and an S2 domain (subunit), wherein the S2 domain (subunit) has been modified to replace the native CT of the S protein with the CT of influenza HA protein and wherein the modified S protein is a single contiguous polypeptide chain. Monomeric or single chain modified S protein may trimerize to form a trimer, referred to as a trimeric modified S protein. A trimer is a macromolecular complex formed by three, usually non-covalently bound proteins.

The S protein is cleaved at a conserved activation cleavage site into 2 polypeptide chains, the S1 subunit and S2 subunit, which remain associated as S1/S2 protomers within the homotrimer. Without wishing to be bound by theory, the cleavage of the S protein into subunits may be important for virus infectivity, but it may not be essential for the trimerization of the protein.

The modified S protein may further comprise one or more than one substitution, replacement or mutation. For example, the modified S protein may comprise one or more than one substitution, replacement or mutation in the ectodomain to increase expression, yield, stability or to increase expression, yield and stability of the modified S protein in a suitable expression system.

For example the modified S protein, may comprise substitutions or mutations to the S1/S2 and/or S2′ protease cleavage sites to prevent protease cleavage at these sites. Therefore, when produced in a host or host cells, the modified S protein is not cleaved into separate S1 and S2 subunits or polypeptide chains.

The modified viral structural protein, such as the modified S protein, may further assemble into trimers of modified viral structural protein. It is therefore further provided a Coronavirus protein trimer comprising the modified S protein as described herein. The trimer may comprise single chain modified S protein wherein the single chain modified S protein comprises an S1 subunit and an S2 subunit, wherein the CT of the S2 subunit has been replaced with the CT of influenza hemagglutinin (HA).

The trimer may further be stabilized in a prefusion conformation. The modified viral structural protein, such as the modified S protein, therefore may further comprise one or more than one substitution, replacement or mutation to inhibit a conformational change in the S protein from the prefusion conformation to the post-fusion conformation, and thereby stabilizing the S protein or S protein trimer in the prefusion conformation.

By “amino acid substitution” or “substitution” it is meant the replacement of an amino acid in the amino acid sequence of a protein with a different amino acid. The terms amino acid, amino acid residue or residue are used interchangeably in the disclosure. One or more amino acids may be replaced with or substituted with one or more amino acids that are different than the original or wild-type amino acid at this position, without changing the overall length of the amino acid sequence of the protein.

For example, the modified viral structural protein, such as the modified S protein may be stabilized by proline substitutions, substitutions allowing the formation of disulfide bonds and salt bridges, and/or cavity-filling substitutions.

Hsieh et al. (Science 2020, 369 p. 1501-1505 which is incorporated herein by reference) designed and expressed a variety of SARS-CoV-2 spike protein variants in mammalian cells. An S protein variant with six proline substitutions, referred to as HexaPro, expressed 9.8× higher than S protein compared to variant that only had a double proline substitutions, had ˜5° C. increase in Tm, and retained the trimeric prefusion conformation in mammalian cell lines. The HexaPro variant is considered the best variant by Hsieh et al.

In the current disclosure, the highest yields were observed with combinations of four proline substitutions corresponding to positions 802, 927, 971 and 972 (“4P”) of SEQ ID NO: 2 and an additional single amino acid substitution at position 923. Furthermore, higher yields were also observed with combinations of six proline substitutions corresponding to positions 802, 877, 884, 927, 971 and 972 (“6P”) and an additional single amino acid substitution at position 923.

As provided herewith, the modified S protein may further comprise one or more than one substitution, replacement or mutation to increase stability, yield or stability and yield of the modified protein in a host or cost cell, such for example in a plant or plant cells.

The modified S protein as described herein may comprise one or more than one mutation, modification, or substitution in its amino acid sequence at any one or more amino acid that corresponds to an amino acid within a reference sequence as described below.

By “correspond to an amino acid”, “corresponding to an amino acid” “or “corresponding to the sequence” and the like, it is meant that an amino acid (or nucleotide) corresponds to an amino acids (or nucleotide) in a sequence alignment with a reference Coronavirus sequence as described below. The corresponding amino acid positions in Coronavirus sequence may be determined by alignment to known sequences of Coronavirus S protein. Methods of alignment of sequences for comparison are well-known in the art and are further described below. Examples of corresponding amino acids are shown in Table 3.

TABLE 3
Positions of corresponding amino acid/residue position in Coronavirus
S-proteins. (Reference sequences are indicated).

| | Amino acid position in S-protein (with ref. to indicated sequence) | | | | | | | | |
|---|---|---|---|---|---|---|---|---|---|
| SARS-CoV-2¹ | 667 | 668 | 670 | 802 | 877 | 884 | 927 | 971 | 972 |
| (SEQ ID NO: 2) | | | | | | | | | |
| SARS-CoV-2² | 682 | 683 | 685 | 817 | 892 | 899 | 942 | 986 | 987 |
| (SEQ ID NO: 1) | | | | | | | | | |
| SARS-CoV-1¹ | 651 | 652 | 654 | 786 | 861 | 868 | 911 | 955 | 956 |
| (SEQ ID NO: 114) | | | | | | | | | |
| SARS-CoV-1² | 664 | 665 | 667 | 799 | 874 | 881 | 924 | 968 | 969 |
| (SEQ ID NO: 112) | | | | | | | | | |
| MERS-CoV¹ | 730 | 731 | 733 | 872 | 949 | 956 | 999 | 1043 | 1044 |
| (SEQ ID NO: 115) | | | | | | | | | |
| MERS-CoV² | 747 | 748 | 750 | 889 | 966 | 973 | 1016 | 1060 | 1061 |
| (SEQ ID NO: 113) | | | | | | | | | |
| OC43-CoV¹ | 748 | 749 | 751 | 898 | N/A | 976 | 1019 | 1063 | 1064 |
| (SEQ ID NO: 160) | | | | | | | | | |
| OC43-CoV² | 762 | 763 | 765 | 912 | N/A | 990 | 1033 | 1077 | 1078 |
| (SEQ ID NO: 158) | | | | | | | | | |
| 229E-CoV¹ | 551 | 552 | 554 | 675 | N/A | 754 | 811 | 855 | 856 |
| (SEQ ID NO: 161) | | | | | | | | | |
| 229E-CoV² | 567 | 568 | 570 | 691 | N/A | 770 | 827 | 871 | 872 |
| (SEQ ID NO: 159) | | | | | | | | | |

¹numbering excludes signal peptide (SP)
²numbering includes signal peptide (SP)

For example, the modified S protein may have one or more than one (for example two consecutive) proline substitutions at or near the boundary between a HR1 domain and a central helix domain that stabilize the S ectodomain trimer in the prefusion conformation, as described for example in WO 2018/081318, which is herein incorporated by reference. Furthermore, the one or more than one substitution may restrict and/or may prevent the processing or cleavage at the cleavage site between the S1 and the S2 subunit.

The modified S protein may comprise one or more than one substitution at a position as indicated in Table 3. For example the modified S protein may comprise one or more than one substitution at a position that corresponds to position 667, 668, 670, 802, 877, 884, 923, 927, 971, 972, or a combination thereof in reference sequence of SEQ ID NO: 2 (SARS-CoV-2). Corresponding positions in S-proteins of SARS-CoV-1, MERS-CoV, OC43-CoV and 229E-CoV are indicated in Table 3. Corresponding amino acid positions in S-protein from other Coronavirus may be determined by methods know within the art.

### GSAS-2P (971 and 972)

For example, the modified S protein may have one or more than one substitution at one or more than one amino acid corresponding to amino acid at positions 667, 668, 670, 971 or 972 of amino acid sequence of SEQ ID NO: 2.

In one aspect, the modified S protein may comprise a substitution, modification or mutation, corresponding to positions 667, 668, 670 or a combination thereof (numbering in accordance with SEQ ID NO: 2). For example, the amino acid corresponding to position 667 may be substituted for glycine (G) or a conserved substitution of glycine (G), the amino acid corresponding to position 668 may be substituted for serine (S) or a conserved substitution of serine (S), and the amino acid corresponding to position 670 may be substituted for serine (S) or a conserved substitution of serine (S).

The modified S protein may further comprise a substitution, modification or mutation, corresponding to positions 971, 972 or at positions 971 and 972 (numbering in accordance with SEQ ID NO: 2). For example, the amino acid corresponding to position 971 and/or 972 may be substituted for proline (P) or a conserved substitution of proline (P).

The modified S protein may comprise one or more than one substitution wherein the one or more than one substitutions comprise or consist of one or more than one substitution of an amino acid corresponding to amino acid at positions 667, 668, 670, 971, 972 of SEQ ID NO: 2. The modified S protein with one or more than one substitutions may be stabilized in a prefusion confirmation. Furthermore, the modified S protein may form trimer that are stabilized in a prefusion confirmation.

For example, the modified S protein may comprise the following substitutions (numbering in accordance with SEQ ID NO: 2): R667G, R668S, R670S (herein referred to as “GSAS”). The modified S protein may also have the following substitutions (numbering in accordance with SEQ ID NO: 2): K971P and V972P (herein referred to as “2P”). Furthermore the modified S protein may have the following substitutions (numbering in accordance with SEQ ID NO: 2): R667G, R668S, R670S, K971P and V972P (herein referred to as “GSAS-2P”).

For example the modified S protein may have an amino acid sequence that has about 70, 75, 80, 85, 87, 90, 91, 92, 93 94, 95, 96, 97, 98, 99, 100% or any amount therebetween, sequence identity, or sequence similarity, with the amino acid sequence of SEQ ID NO: 47 sequence or with amino acids 25-1259 of SEQ ID NO: 47, wherein the amino acid sequence has glycine (G) or a conserved substitution of glycine (G) at position 667, serine (S) or a conserved substitution of serine (S) at position 668, serine (S) or a conserved substitution of serine (S) at position 670, proline (P) or a conserved substitution of proline (P) at positions 971 and 972, wherein the modified S protein, when expressed, forms VLP.

In another example, the modified S protein may have one or more than one substitution at one or more than one amino acid corresponding to amino acid at positions 654, 955 or 956 of amino acid sequence of SEQ ID NO: 114 or at positions 730, 733, 1043 or 1044 of amino acid sequence of SEQ ID NO: 115.

For example, the modified S protein may comprise the following substitutions: R654A (numbering in accordance with SEQ ID NO: 114) or R730A and/or R733G (numbering in accordance with SEQ ID NO: 115). The modified S protein may also have the following substitutions: K955P and/or V956P (numbering in accordance with SEQ ID NO: 114) or V1043P and/or L1044P (numbering in accordance with SEQ ID NO: 115). Furthermore the modified S protein may have the following substitutions: R654A, K955P and V956P (numbering in accordance with SEQ ID NO: 114) or R730A, R733G, V1043P, L1044P (numbering in accordance with SEQ ID NO: 115).

### GSAS-4P (802, 927, 971 and 972)

The modified S protein may further have substitution at amino acids corresponding to amino acid at positions 667, 668, and 670 and further one or more than one substitution at one or more than one residue corresponding to positions 802, 927, 971 and 972 (numbering in accordance with SEQ ID NO: 2). For example, the amino acid corresponding to positions 802, 927, 971 and 972 may be substituted for proline (P) or a conserved substitution of proline (P).

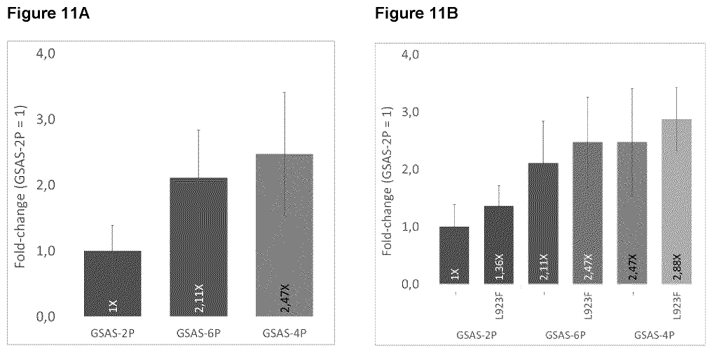

As shown in FIG. 11A, modified S protein having the “GSAS” modifications and the following modifications: F802P, A927P, K971P, V972P (referred to as “GSAS-4P”, expressed from construct 8953) showed an increase of 2.47-fold increase in yield of modified S protein when compared to the yield of the “GSAS-2P” S protein (expressed from construct 8671).

Accordingly, the modified S protein may comprise one or more than one substitution wherein the one or more than one substitution comprise or consist of one or more than one substitution of an amino acid corresponding to amino acid at positions 667, 668, 670, 802, 927, 971 and 972 of SEQ ID NO: 2.

For example the modified S protein may have an amino acid sequence that has about 70, 75, 80, 85, 87, 90, 91, 92, 93 94, 95, 96, 97, 98, 99, 100% or any amount therebetween, sequence identity, or sequence similarity, with the amino acid sequence of SEQ ID NO: 48 or with amino acids 25-1259 of SEQ ID NO: 48, wherein the amino acid sequence has glycine (G) or a conserved substitution of glycine (G) at position 667, serine (S) or a conserved substitution of serine (S) at position 668, serine (S) or a conserved substitution of serine (S) at position 670, proline (P) or a conserved substitution of proline (P) at positions 802, 927, 971 and 972, wherein the modified S protein, when expressed, forms VLP.

In another example, the modified S protein may have one or more than one substitution at one or more than one amino acid corresponding to amino acid at positions 654, 786, 911, 955 or 956 of amino acid sequence of SEQ ID NO: 114 or at positions 730, 733, 872, 999, 1043 or 1044 of amino acid sequence of SEQ ID NO: 115.

For example, the modified S protein may comprise the following substitutions: R654A (numbering in accordance with SEQ ID NO: 114) or R730A and/or R733G (numbering in accordance with SEQ ID NO: 115). The modified S protein may also have the following substitutions: F786P, S911P, K955P and/or V956P (numbering in accordance with SEQ ID NO: 114) or A872P, N999P, V1043P and/or L1044P (numbering in accordance with SEQ ID NO: 115). Furthermore the modified S protein may have the following substitutions: R654A, F786P, S911P, K955P and V956P (numbering in accordance with SEQ ID NO: 114) or R730A, R733G, A872P, N999P, V1043P, L1044P (numbering in accordance with SEQ ID NO: 115).

### GSAS-6P (802, 877, 884, 927, 971 and 972)

The modified S protein may further have substitution at amino acids corresponding to amino acid at positions 667, 668, and 670 and further one or more than one substitution at one or more than one residue corresponding to positions 802, 877, 884, 927, 971, and 972 (numbering in accordance with SEQ ID NO: 2). For example, the amino acid corresponding to position 802, 877, 884, 927, 971, and 972 may be substituted for proline (P) or a conserved substitution of proline (P) (numbering in accordance with SEQ ID NO: 2).

As shown in FIG. 11A, modified S protein having the “GSAS” modifications and the following modifications: F802P, A877P, A884P, A927P, K971P, V972P (referred to as “GSAS-6P”, expressed from construct 8940) showed an increase of 2.11-fold increase in yield of S protein when compared to the yield of the “GSAS-2P” S protein (expressed from construct 8671).

Accordingly, the modified S protein may comprise one or more than one substitution wherein the one or more than one substitution comprise or consist of one or more than one substitution of an amino acid corresponding to amino acid at positions 667, 668, 670, 802, 877, 884, 927, 971 and 972 of SEQ ID NO: 2.

For example the modified S protein may have an amino acid sequence that has about 70, 75, 80, 85, 87, 90, 91, 92, 93 94, 95, 96, 97, 98, 99, 100% or any amount therebetween, sequence identity, or sequence similarity, with the amino acid sequence of SEQ ID NO: 49 or with amino acids 25-1259 of SEQ ID NO: 48, wherein the amino acid sequence has glycine (G) or a conserved substitution of glycine (G) at position 667, serine (S) or a conserved substitution of serine (S) at position 668, serine (S) or a conserved substitution of serine (S) at position 670, proline (P) or a conserved substitution of proline (P) at position 802, 802, 877, 884, 927, 971 and 972, wherein the modified S protein when expressed forms VLP.

In another example, the modified S protein may have one or more than one substitution at one or more than one amino acid corresponding to amino acid at positions 654, 786, 861, 868, 911, 955 or 956 of amino acid sequence of SEQ ID NO: 114 or at positions 730, 733, 872, 949, 956, 999, 1043 or 1044 of amino acid sequence of SEQ ID NO: 115.

For example, the modified S protein may comprise the following substitutions: R654A (numbering in accordance with SEQ ID NO: 114) or R730A and/or R733G (numbering in accordance with SEQ ID NO: 115). The modified S protein may also have the following substitutions: F786P, A861P, A868P, S911P, K955P and/or V956P (numbering in accordance with SEQ ID NO: 114) or A872P, S949P, A956P, N999P, V1043P and/or L1044P (numbering in accordance with SEQ ID NO: 115). Furthermore the modified S protein may have the following substitutions: R654A, F786P, A861P, A868P, S911P, K955P and V956P (numbering in accordance with SEQ ID NO: 114) or R730A, R733G, A872P, S949P, A956P, N999P, V1043P and L1044P (numbering in accordance with SEQ ID NO: 115).

### Substitution at Position 923

The modified S protein as described herewith may further comprise a substitution, modification, or mutation, corresponding to position 923 (numbering in accordance with SEQ ID NO: 2). For example the amino acid corresponding to position 923 may be substituted for phenylalanine (F) or a conserved substitution of phenylalanine (F).

As shown in FIG. 11B, modified S protein having the “GSAS-2P” modifications and a L923F substitution (expressed from construct 8933) showed an increase of 1.36-fold yield of S protein when compared to the yield of the “GSAS-2P” S protein without the L923F substitution (expressed from construct 8671). The modified S protein having the “GSAS-4P” modifications and a L923F substitution (expressed from construct 8960) showed an increase of 2.88-fold in yield of the modified S protein when compared to the yield of the “GSAS-2P” S protein without the L923F substitution (expressed from construct 8671). The modified S protein having the “GSAS-6P” modifications and a L923F substitution (expressed from construct 8947) showed an increase of 2.47-fold in yield when compared to the yield of the “GSAS-2P” S protein without the L923F substitution (expressed from construct 8671).

Accordingly, the modified S protein may comprise one or more than one substitution wherein the one or more than one substitution comprises or consists of one or more than one substitution of an amino acid corresponding to amino acids at positions 667, 668, 670, 927, 971, 972, 802, 877, 884, 923 or a combination thereof of SEQ ID NO: 2. For example the modified S-protein may comprise one or more than one substitution wherein the one or more than one substitution comprises or consists of one or more than one substitution of an amino acid corresponding to amino acids at positions 667, 668, 670, 971, 972, 923, or a combination thereof of SEQ ID NO: 2 (GSAS-2P-923), 667, 668, 670, 927, 971, 972, 802 923, or a combination thereof of SEQ ID NO: 2 (GSAS-4P-923) or 667, 668, 670, 927, 971, 972, 802, 877, 884, 923 or a combination thereof of SEQ ID NO: 2 of SEQ ID NO: 2 (GSAS-6P-923).

For example, the modified S protein may comprise one or more than one substitution wherein the one or more than one substitution comprises or consists of one or more than one substitution of an amino acid corresponding to amino acids at positions
667, 668, 670, 971, 972 and 923 of SEQ ID NO: 2 (GSAS-2P-923),
667, 668, 670, 927, 971, 972, 802 and 923 of SEQ ID NO: 2 (GSAS-4P-923) or
667, 668, 670, 927, 971, 972, 802, 877, 884 and 923 of SEQ ID NO: 2 (GSAS-6P-923).

For example, the modified S protein may have an amino acid sequence that has about 70, 75, 80, 85, 87, 90, 91, 92, 93 94, 95, 96, 97, 98, 99, 100% or any amount therebetween, sequence identity, or sequence similarity, with the amino acid sequence of SEQ ID NO: 50 or amino acids 25-1259 of SEQ ID NO: 50, wherein the amino acid sequence has glycine (G) or a conserved substitution of glycine (G) at position 667, serine (S) or a conserved substitution of serine (S) at positions 668 and 670, proline (P) or a conserved substitution of proline (P) at positions 971 and 972, and phenylalanine (F) or a conserved substitution of phenylalanine (F) at position 923; SEQ ID NO: 51 or amino acids 25-1259 of SEQ ID NO: 51, wherein the amino acid sequence has glycine (G) or a conserved substitution of glycine (G) at position 667, serine (S) or a conserved substitution of serine (S) at positions 668 and 670, proline (P) or a conserved substitution of proline (P) at positions 927, 971, 972 and 802, and phenylalanine (F) or a conserved substitution of phenylalanine (F) at position 923; or SEQ ID NO: 52 or amino acids 25-1259 of SEQ ID NO: 52, wherein the amino acid sequence has glycine (G) or a conserved substitution of glycine (G) at position 667, serine (S) or a conserved substitution of serine (S) at positions 668 and 670, proline (P) or a conserved substitution of proline (P) at positions 927, 971, 972, 802, 877 and 884, and phenylalanine (F) or a conserved substitution of phenylalanine (F) at position 923, wherein the modified S protein when expressed forms VLP.

Accordingly, it is provided a modified coronavirus S-protein which may comprise:
1. a chimeric transmembrane and cytoplasmic tail domain (TMCT);
2. one or more than one substitution corresponding to amino acid at positions 667, 668, and/or 670 (numbering in accordance with SEQ ID NO: 2) when compared to a corresponding wildtype coronavirus S-protein;
3. one or more than one substitution corresponding to amino acid at positions 802, 877, 884, 927, 971, and/or 972 (numbering in accordance with SEQ ID NO: 2) when compared to a corresponding wildtype coronavirus S-protein;
4. a substitution corresponding to position 923 (numbering in accordance with SEQ ID NO: 2) when compared to a corresponding wildtype coronavirus S-protein;
5. or a combination of the modifications and/or substitutions as described under 1-4.

As used herein, the term “conserved substitution” or “conservative substitution” and grammatical variations thereof, refers to an amino acid that is different from an reference amino acid (substitution), but is in the same class of amino acid as the described substitution or described residue (i.e., a nonpolar residue replacing a nonpolar residue, an aromatic residue replacing an aromatic residue, a polar-uncharged residue replacing a polar-uncharged residue, a charged residue replacing a charged residue). Further information about conservative substitutions can be found, for instance, in Sahin-Toth et al. (Protein ScL, 3:240-247, 1994), Hochuli et al (Bio/Technology, 6:1321-1325, 1988) Henikoff S, and Henikoff JG (Proc. Natl. Acad. Sci. USA 89: 10915-10919, 1992) and in widely used textbooks of genetics and molecular biology.

The modified viral structural protein may further be glycosylated. Coronavirus S protein, Coronavirus M protein and Coronavirus E protein are glycosylated and both N-linked glycosylation and O-linked glycosylation occur.

The modified viral structural protein may comprise glycosylation pattern that are unique to the host or host cell in which the modified viral structural protein is expressed. For example, when expressed in plants or plant cells, the modified viral structural protein may comprise plant-specific N-glycans. Therefore, it is also provided modified viral structural protein having plant specific N-glycans.

As described herein, the cytosolic tail domain (CT) of the modified viral structural protein may be replaced with the CT from influenza hemagglutinin (HA). The ectodomain and the transmembrane domain (TM) of the viral structural protein as described above are fused to an influenza HA cytosolic tail domain (CT) such that the CT is heterologous with respect to the ectodomain and the transmembrane domain of the viral structural protein, such as the S protein. The modified S protein may self-assemble into virus-like particles (VLPs).

The present description therefore further relates to virus-like particles (VLPs). More specifically, the present description is directed to VLPs comprising modified viral structural proteins such as modified S-protein, and methods of producing VLPs with modified viral structural proteins such as modified S-protein in a host or host cell. The VLPs comprise a modified viral structural protein such as modified S-protein as described herewith.

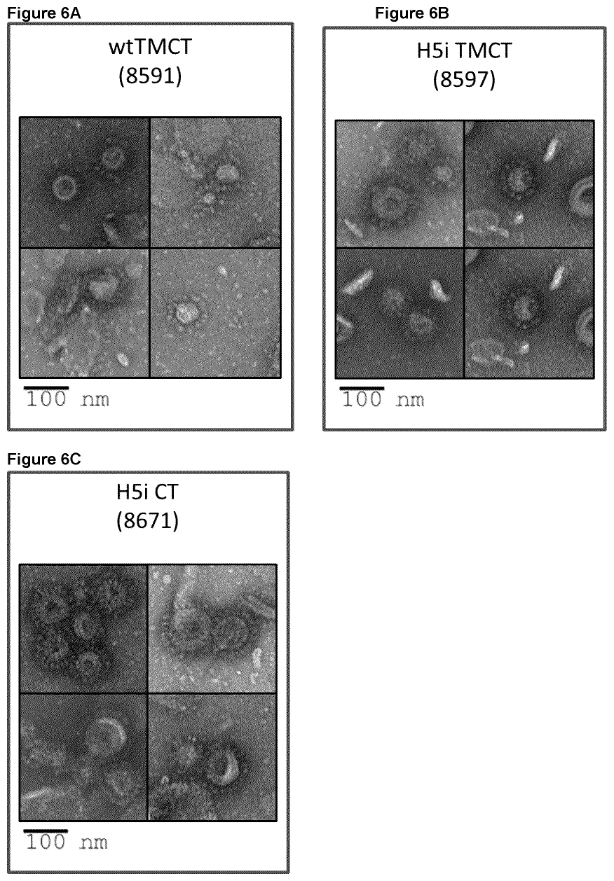

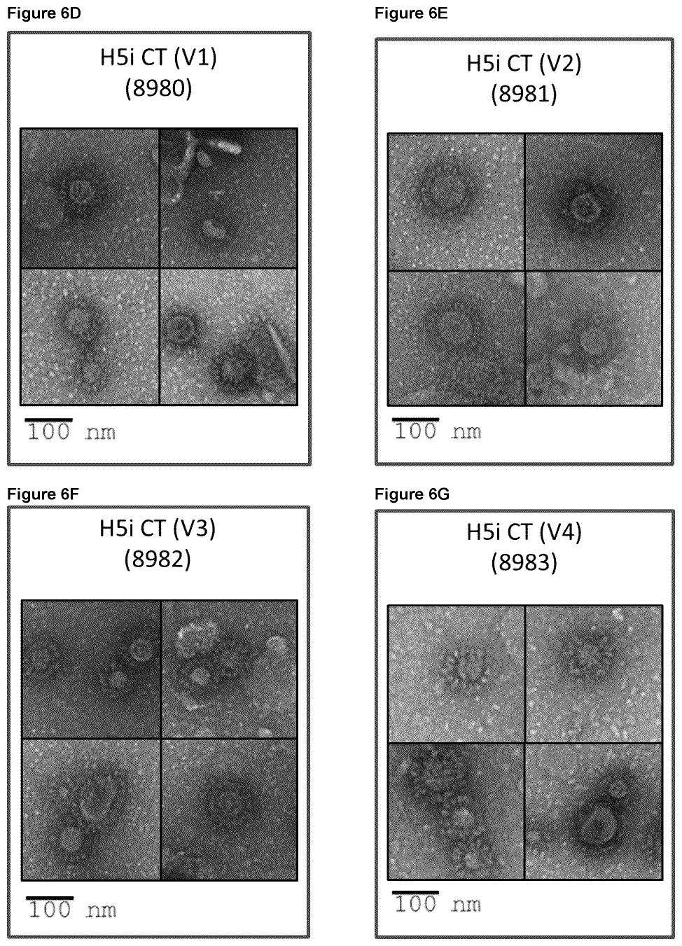

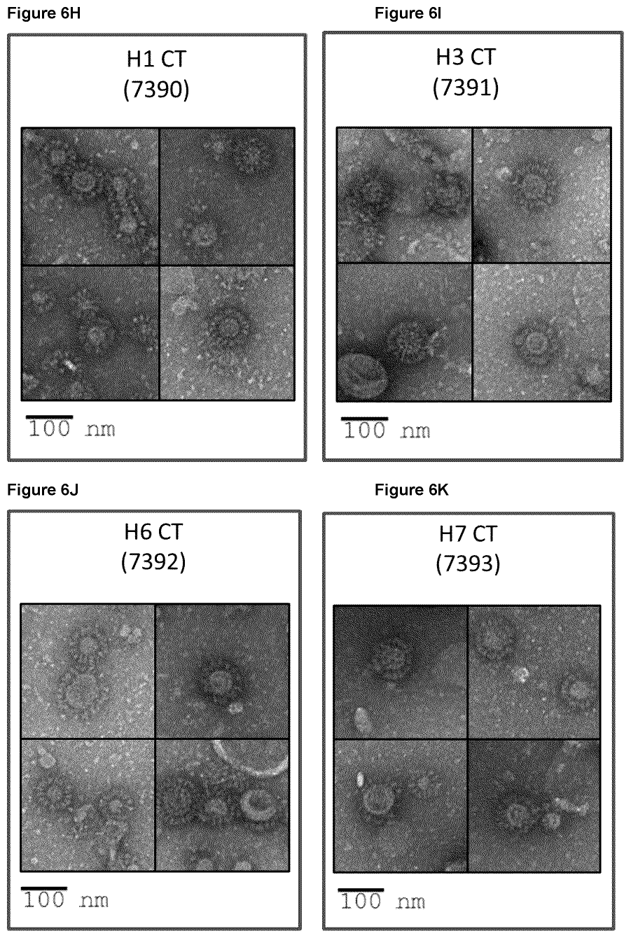

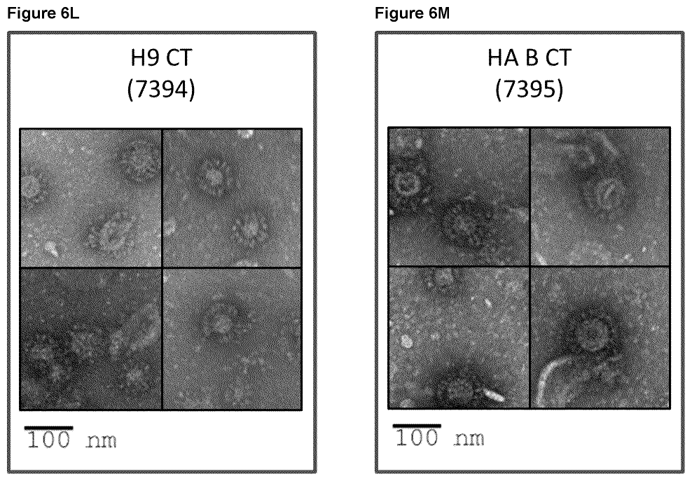

As shown in FIGS. 6C, 17A, 17B, and 17C, modified viral structural protein as exemplified by a modified S protein (modified SARS-CoV-2 or modified SARS-CoV-1 S protein), wherein the native or wild-type CT has been replaced by a CT from influenza HA protein self-assemble into VLPs when expressed in plants. The VLPs are similar to VLPs produced with a S protein with native TM/CT sequence (see FIGS. 6A and 17A) or modified S protein with H5 influenza TM/CT sequence (see FIGS. 6B and 17B) in the same plant expression system.

Furthermore, as shown in FIGS. 6D, 6E, 6F and 6G, modified S protein with variable margin or boundaries (intervening peptide sequence) between the TM and influenza CT domain also self-assemble into VLPs when expressed in plants.

In addition, as shown in FIGS. 6H, 6I, 6J, 6K, 6L and 6M, modified S protein, wherein the native or wild-type CT has been replaced by a CT from influenza HA protein from H1, H3, H6, H7, H9 and B influenza, respectively, also self-assemble into VLPs when expressed in plants.

Furthermore, as shown in FIGS. 19B-19F, 23B-23E, and 25A-25E, modified S-protein derived from MERS-CoV, OC43-CoV, and 229E-CoV, wherein the modified S-protein has a TMCT from influenza H5 HA (H5iTMCT), a CT from influenza H5 HA (H5iCT), or a CT from influenza H1 HA also formed VLPs.

The term virus-like particle” (VLP), or “virus-like particles” or “VLPs” refers to virus-like structures that are generally morphologically and antigenically similar to virions produced in an infection, but lack genetic information sufficient to replicate and thus are non-infectious. VLPs are structures that self-assemble and comprise one or more structural proteins such as for example modified viral structural proteins, for example but not limited to a modified S protein. Therefore, the VLP may comprise modified S protein. The VLP may further comprise viral structural proteins, wherein the viral structural proteins consist of modified S protein. Therefore, in some embodiments the VLP may lack or be free of the Coronavirus M protein and/or Coronavirus E protein. In some embodiments the VLPs produced from the modified viral structural protein as described herewith, therefore do not comprise a Coronavirus M protein, a Coronavirus E protein or Coronavirus M protein and Coronavirus E protein. Furthermore, in some embodiment the VLP do not comprise structural or non-structural proteins from viruses that are heterologous to Coronaviridae or influenza virus, for example the VLP do not comprise structural and non-structural protein from viruses that are not from Coronaviridae.

In another embodiment the VLP may comprise Coronavirus E protein, Coronavirus M protein and modified Coronavirus S protein. In another embodiment the VLP may comprise Coronavirus E protein and modified Coronavirus S protein. In another embodiment the VLP may comprise Coronavirus M protein and modified Coronavirus S protein. Furthermore, the VLP may comprise Coronavirus E protein, modified Coronavirus M protein and modified Coronavirus S protein. The VLP may further comprise modified Coronavirus E protein, modified Coronavirus M protein and modified Coronavirus S protein. In another embodiment the VLP may comprise modified Coronavirus E protein and modified Coronavirus S protein. In another embodiment the VLP may comprise modified Coronavirus M protein and modified Coronavirus S protein.

VLPs may be produced in suitable host or host cells including plants and plant cells. Following extraction from the host or host cell and upon isolation and further purification under suitable conditions, VLPs may be recovered as intact structures.

The VLPs may be purified or extracted using any suitable method for example chemical or biochemical extraction. VLPs are relatively sensitive to desiccation, heat, pH, surfactants and detergents. Therefore it may be useful to use methods that maximize yields, minimize contamination of the VLP fraction with cellular proteins, maintain the integrity of the proteins, or VLPs, and, where required, the associated lipid envelope or membrane, methods of loosening the cell wall to release the proteins, or VLP. Minimizing or eliminating the use of detergence or surfactants such for example SDS or Triton™ X-100 may be beneficial for improving the yield of VLP extraction. VLPs may be then assessed for structure and size by, for example, electron microscopy (see FIG. 4B), or by size exclusion chromatography.

For enveloped viruses, such as Coronavirus, it may be advantageous for a lipid layer or membrane to be retained by the virus. The composition of the lipid may vary with the system (e.g. a plant-produced enveloped virus would include plant lipids or phytosterols in the envelope), and may contribute to an improved immune response.

Therefore, the VLPs that are produced in a host or host cell, may comprise lipids from the plasma membrane of the host or host cell. For example VLPs produced in plants may contain lipids of plant origin (“plant lipids”), VLPs produced in insect cells may comprise lipids from the plasma membrane of insect cells (generally referred to as “insect lipids”), and VLPs produced in mammalian cells may comprise lipids from the plasma membrane of mammalian cells (generally referred to as “mammalian lipids”).

The plant lipids or plant-derived lipids may be in the form of a lipid bilayer, and may further comprise an envelope surrounding the VLP. The plant-derived lipids may comprise lipid components of the plasma membrane of the plant where the VLP is produced, including phospholipids, tri-, di- and monoglycerides, as well as fat-soluble sterol or metabolites comprising sterols. Examples include phosphatidylcholine (PC), phosphatidylethanolamine (PE), phosphatidylinositol, phosphatidylserine, glycosphingolipids, phytosterols or a combination thereof. Examples of phytosterols include campesterol, stigmasterol, ergosterol, brassicasterol, delta-7-stigmasterol, delta-7-avenasterol, daunosterol, sitosterol, 24-methylcholesterol, cholesterol or beta-sitosterol. As one of skill in the art would understand, the lipid composition of the plasma membrane of a cell may vary with the culture or growth conditions of the cell or organism, or species, from which the cell is obtained. Generally, beta-sitosterol is the most abundant phytosterol.

Without wishing to be bound by theory, plant-made VLPs comprising plant derived lipids, may induce a stronger immune reaction than VLPs made in other manufacturing systems and the immune reaction induced by these plant-made VLPs may be stronger when compared to the immune reaction induced by live or attenuated whole virus vaccines.

Furthermore, in addition to the potential adjuvant effect of the presence of plant lipids, the ability of plant N-glycans to facilitate the capture of glycoprotein antigens by antigen presenting cells, may be advantageous of the production of VLPs in plants.

The VLP produced within a plant may comprise a modified viral structural protein comprising plant-specific N-glycans. Therefore, this disclosure also provides for a VLP comprising modified viral structural protein having plant specific N-glycans. Furthermore, it is provided VLP comprising plant lipids and modified viral structural protein having plant specific N-glycans.

Methods of producing virus like particle (VLP) comprising modified structural protein in a host or host cell are also provided. Furthermore, methods of increasing yield of production of virus like particle (VLP) comprising modified structural protein in a host or host cell are also provided. The methods comprise the introduction of a nucleic acid comprising a sequence that encodes a modified structural protein into the host or host cell, and incubating the host or host cell under conditions that permit the expression of the nucleic acid, thereby producing the VLP. The modified viral structural protein may be produced at a higher yield compared to a host or host cell expressing the unmodified viral structural protein.

For example, as shown in FIG. 3A, yields of VLPs expressed in plants may be increased when the cytoplasmic tail (CT) of a viral structural protein is replaced with the CT of influenza HA to produce a modified viral structural protein, such for example a modified S protein. As further shown in FIGS. 11A and 11B, when the modified S protein further comprises one or more than one substitution wherein the one or more than one substitution comprise or consist of one or more than one substitution of an amino acid corresponding to amino acid at positions 667, 668, 670, 802, 923, 927, 971 and/or 972 of SEQ ID NO: 2, yield of VLPs comprising the modified S protein when expressed in plants, may be further increased.

The yield of the modified viral structural protein (such as modified S protein) or the yield of a VLP comprising modified viral structural protein produced in a host or host cell, such for example a plant or plant cells, may be increased by 1.1-10 fold, or any amount therebetween when compared to the yield of a corresponding unmodified viral structural protein or the yield of VLP that comprises the corresponding unmodified viral structural protein. For example the yield of the modified viral structural protein (such as modified S protein) or the yield of a VLP (comprising the modified viral structural protein) in a host or host cell may be increased by 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.1, 5.2, 5.3, 5.4, 5.5, 5.6, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9 9.1, 9.2, 9.3, 9.4, 9.5, 9.6, 9.7, 9.8, 9.9, 10 fold or any amount therebetween, compared to the yield of a corresponding unmodified viral structural protein or the yield of a VLP wherein the VLP comprises a corresponding unmodified viral structural protein, when produced in a host or host cell under identical conditions.

The modified viral structural protein described herewith includes modified S proteins with amino acid sequences that have about 70, 75, 80, 85, 87, 90, 91, 92, 93 94, 95, 96, 97, 98, 99, 100% or any amount therebetween, sequence identity, or sequence similarity, with the amino acid sequence of SEQ ID NO: 1, 2, 5, 21, 30, 47, 48, 49, 50, 51, 52, 53, 54, 55, 56, 57, 58, 59, 60, 61, 62, 95, 96, 97, 108, 109, 110, 112, 113, 114, 115, 144, 145, 146, 155, 156, 157, 158, 159, 160, or 161, or with amino acids 25-1259 of SEQ ID NO: 47, amino acids 25-1259 of SEQ ID NO: 48, amino acids 25-1259 of SEQ ID NO: 49, amino acids 25-1259 of SEQ ID NO: 50, amino acids 25-1259 of SEQ ID NO: 51, amino acids 25-1259 of SEQ ID NO: 52, amino acids 25-1259 of SEQ ID NO: 53, amino acids 25-1259 of SEQ ID NO: 54, amino acids 25-1259 of SEQ ID NO: 55, amino acids 25-1259 of SEQ ID NO: 56, amino acids 25-1259 of SEQ ID NO: 57, amino acids 25-1259 of SEQ ID NO: 58, amino acids 25-1262 of SEQ ID NO: 59, amino acids 25-1261 of SEQ ID NO: 60, amino acids 25-1258 of SEQ ID NO: 61, amino acids 25-1256 of SEQ ID NO: 62, amino acids 25-1243 of SEQ ID NO: 95, amino acids 25-1240 of SEQ ID NO: 96, amino acids 25-1243 of SEQ ID NO: 97, amino acids 25-1341 of SEQ ID NO: 108, amino acids 25-1338 of SEQ ID NO: 109, amino acids 25-1341 of SEQ ID NO: 110, amino acids 25-1351 of SEQ ID NO: 144, amino acids 25-1348 of SEQ ID NO: 145, amino acids 25-1351 of SEQ ID NO: 146, amino acids 25-1159 of SEQ ID NO: 155, amino acids 25-1156 of SEQ ID NO: 156, or amino acids 25-1159 of SEQ ID NO: 157, and wherein modified S proteins when expressed in a host or host cell form VLP. The amino acid sequence of the ectodomain and the transmembrane domain of the modified S proteins has about 70, 75, 80, 85, 87, 90, 91, 92, 93 94, 95, 96, 97, 98, 99, 100% or any amount therebetween sequence identity, or sequence similarity, with amino acids 1-1234 of SEQ ID NO:1, with amino acids 1-1219 of SEQ ID NO: 2, with amino acids 1-1234 of SEQ ID NO: 5, with amino acids 1-1219 of SEQ ID NO: 21, with amino acids 1-1243 of SEQ ID NO: 30, with the amino acids 25-1243 of SEQ ID NO: 47, with the amino acids 25-1243 of SEQ ID NO: 48, with the amino acids 25-1243 of SEQ ID NO: 49, with the amino acids 25-1243 of SEQ ID NO: 50, with the amino acids 25-1243 of SEQ ID NO: 51, with the amino acids 25-1243 of SEQ ID NO: 52, with the amino acids 25-1243 of SEQ ID NO: 53, with the amino acids 25-1243 of SEQ ID NO: 54, with the amino acids 25-1243 of SEQ ID NO: 55, with the amino acids 25-1243 of SEQ ID NO: 56, with the amino acids 25-1243 of SEQ ID NO: 57, with the amino acids 25-1243 of SEQ ID NO: 58, with the amino acids 25-1242 of SEQ ID NO: 59, with the amino acids 25-1242 of SEQ ID NO: 60, with the amino acids 25-1246 of SEQ ID NO: 61, or with the amino acids 25-1245 of SEQ ID NO: 62, amino acids 25-1227 of SEQ ID NO: 95, amino acids 25-1227 of SEQ ID NO: 96, amino acids 25-1227 of SEQ ID NO: 97, amino acids 25-1325 of SEQ ID NO: 108, amino acids 25-1325 of SEQ ID NO: 109, amino acids 25-1325 of SEQ ID NO: 110, amino acids 25-1335 of SEQ ID NO: 144, amino acids 25-1335 of SEQ ID NO: 145, amino acids 25-1335 of SEQ ID NO: 146, amino acids 25-1143 of SEQ ID NO: 155, amino acids 25-1143 of SEQ ID NO: 156, or amino acids 25-1143 of SEQ ID NO: 157, and the amino acid sequence of the cytoplasmic tail domain (CT) of the modified S protein has about 70, 75, 80, 85, 87, 90, 91, 92, 93 94, 95, 96, 97, 98, 99, 100% or any amount therebetween sequence identity, or sequence similarity, with the sequence of SEQ ID NO: 15, or with amino acids 35-50 of SEQ ID NO 6, 8, 7, 9, 10, 12, 13, 14, or with amino acids 34-49 of SEQ ID NO 11, or with amino acids 553-568 of SEQ ID NO:3 and wherein modified S proteins when expressed in a host or host cell form VLP.

Furthermore, the modified viral structural protein may be encoded by a nucleotide sequence that has about 70, 75, 80, 85, 87, 90, 91, 92, 93 94, 95, 96, 97, 98, 99, 100% or any amount therebetween, sequence identity, or sequence similarity, with the nucleotide sequence according to SEQ ID NO: 22, 26, 29, 70, 71, 72, 73, 74, 75, 76, 77, 78, 79, 90, 91, 92, 95, 96, 97, 103, 104, 105, 139, 140, 141, 150, 151, or 152 and wherein the nucleotide sequence encodes modified S proteins that when expressed in a host or host cell form VLP.

It is further provided nucleotide sequence encoding a modified S proteins with amino acid sequences that have about 70, 75, 80, 85, 87, 90, 91, 92, 93 94, 95, 96, 97, 98, 99, 100% or any amount therebetween, sequence identity, or sequence similarity, with the amino acid sequence of SEQ ID NO: 1, 2, 5, 21, 30, 47, 48, 49, 50, 51, 52, 53, 54, 55, 56, 57, 58, 59, 60, 61, 62, 95, 96, 97, 108, 109, 110, 144, 145, 146, 155, 156 or 157, and wherein modified S proteins when expressed in a host or host cell form VLP. The nucleotide sequence may encode an amino acid sequence of the ectodomain and the transmembrane domain of the modified S proteins that has about 70, 75, 80, 85, 87, 90, 91, 92, 93 94, 95, 96, 97, 98, 99, 100% or any amount therebetween sequence identity, or sequence similarity, with amino acids 1-1234 of SEQ ID NO:1, with amino acids 1-1219 of SEQ ID NO: 2, with amino acids 1-1234 of SEQ ID NO: 5, with amino acids 1-1219 of SEQ ID NO: 21 or with amino acids 1-1243 of SEQ ID NO: 30, with the amino acids 25-1243 of SEQ ID NO: 47, with the amino acids 25-1243 of SEQ ID NO: 48, with the amino acids 25-1243 of SEQ ID NO: 49, with the amino acids 25-1243 of SEQ ID NO: 50, with the amino acids 25-1243 of SEQ ID NO: 51, with the amino acids 25-1243 of SEQ ID NO: 52, with the amino acids 25-1243 of SEQ ID NO: 53, with the amino acids 25-1243 of SEQ ID NO: 54, with the amino acids 25-1243 of SEQ ID NO: 55, with the amino acids 25-1243 of SEQ ID NO: 56, with the amino acids 25-1243 of SEQ ID NO: 57, with the amino acids 25-1243 of SEQ ID NO: 58, with the amino acids 25-1242 of SEQ ID NO: 59, with the amino acids 25-1242 of SEQ ID NO: 60, with the amino acids 25-1246 of SEQ ID NO: 61, with the amino acids 25-1245 of SEQ ID NO: 62, amino acids 25-1227 of SEQ ID NO: 95, amino acids 25-1227 of SEQ ID NO: 96, amino acids 25-1227 of SEQ ID NO: 97, amino acids 25-1325 of SEQ ID NO: 108, amino acids 25-1325 of SEQ ID NO: 109, amino acids 25-1325 of SEQ ID NO: 110, amino acids 25-1335 of SEQ ID NO: 144, amino acids 25-1335 of SEQ ID NO: 145, amino acids 25-1335 of SEQ ID NO: 146, amino acids 25-1143 of SEQ ID NO: 155, amino acids 25-1143 of SEQ ID NO: 156, or amino acids 25-1143 of SEQ ID NO: 157, and the amino acid sequence of the cytoplasmic tail domain of the modified S protein has about 70, 75, 80, 85, 87, 90, 91, 92, 93 94, 95, 96, 97, 98, 99, 100% or any amount therebetween sequence identity, or sequence similarity, with the sequence of SEQ ID NO: 15, or with amino acids 35-50 of SEQ ID NO 6, 8, 7, 9, 10, 12, 13, 14, or with amino acids 34-49 of SEQ ID NO 11, or with amino acids 553-568 of SEQ ID NO:3 and wherein modified S proteins when expressed in a host or host cell form VLP.

It is further provided a nucleotide sequence encoding a modified S proteins with amino acid sequences that have about 70, 75, 80, 85, 87, 90, 91, 92, 93 94, 95, 96, 97, 98, 99, 100% or any amount therebetween, sequence identity, or sequence similarity, with the amino acid sequence of SEQ ID NO: 5, 21, 30, or 47-62, or with amino acids 24-1259 of SEQ ID NO: 47 amino acids 25-1259 of SEQ ID NO: 48, amino acids 25-1259 of SEQ ID NO: 49, amino acids 25-1259 of SEQ ID NO: 50, amino acids 25-1259 of SEQ ID NO: 51, amino acids 25-1259 of SEQ ID NO: 52, amino acids 25-1259 of SEQ ID NO: 53, amino acids 25-1259 of SEQ ID NO: 54, amino acids 25-1259 of SEQ ID NO: 55, amino acids 25-1259 of SEQ ID NO: 56, amino acids 25-1259 of SEQ ID NO: 57, amino acids 25-1259 of SEQ ID NO: 58, amino acids 25-1262 of SEQ ID NO: 59, amino acids 25-1261 of SEQ ID NO: 60, amino acids 25-1258 of SEQ ID NO: 61, or amino acids 25-1256 of SEQ ID NO: 62, amino acids 25-1243 of SEQ ID NO: 95, amino acids 25-1240 of SEQ ID NO: 96, amino acids 25-1243 of SEQ ID NO: 97, amino acids 25-1341 of SEQ ID NO: 108, amino acids 25-1338 of SEQ ID NO: 109, amino acids 25-1341 of SEQ ID NO: 110, amino acids 25-1351 of SEQ ID NO: 144, amino acids 25-1348 of SEQ ID NO: 145, amino acids 25-1351 of SEQ ID NO: 146, amino acids 25-1159 of SEQ ID NO: 155, amino acids 25-1156 of SEQ ID NO: 156, or amino acids 25-1159 of SEQ ID NO: 157, and wherein modified S proteins when expressed in a host or host cell form VLP.

The terms “percent similarity”, “sequence similarity”, “percent identity”, or “sequence identity”, when referring to a particular sequence, are used for example as set forth in the University of Wisconsin GCG software program, or by manual alignment and visual inspection (see, e.g., Current Protocols in Molecular Biology, Ausubel et al., eds. 1995 supplement). Methods of alignment of sequences for comparison are well-known in the art. Optimal alignment of sequences for comparison can be conducted, using for example the algorithm of Smith & Waterman, (1981, Adv. Appl. Math. 2:482), by the alignment algorithm of Needleman & Wunsch, (1970, J. Mol. Biol. 48:443), by the search for similarity method of Pearson & Lipman, (1988, Proc. Natl. Acad. Sci. USA 85:2444), by computerized implementations of these algorithms (for example: GAP, BESTFIT, FASTA, and TFASTA in the Wisconsin Genetics Software Package, Genetics Computer Group (GCG), 575 Science Dr., Madison, Wis.).

An example of an algorithm suitable for determining percent sequence identity and sequence similarity are the BLAST and BLAST 2.0 algorithms, which are described in Altschul et al., (1977, Nuc. Acids Res. 25:3389-3402) and Altschul et al., (1990, J. Mol. Biol. 215:403-410), respectively. BLAST and BLAST 2.0 are used, with the parameters described herein, to determine percent sequence identity for the nucleic acids and proteins of the disclosure. For example the BLASTN program (for nucleotide sequences) may use as defaults a wordlength (W) of 11, an expectation (E) of 10, M=5, N=−4 and a comparison of both strands. For amino acid sequences, the BLASTP program may use as defaults a word length of 3, and expectation (E) of 10, and the BLOSUM62 scoring matrix (see Henikoff & Henikoff, 1989, Proc. Natl. Acad. Sci. USA 89:10915) alignments (B) of 50, expectation (E) of 10, M=5, N=−4, and a comparison of both strands. Software for performing BLAST analyses is publicly available through the National Center for Biotechnology Information (see URL: ncbi.nlm.nih.gov/).

A nucleic acid sequence or nucleotide sequence referred to in the present disclosure, may be “substantially homologous”, “substantially similar” or “substantially identical” to a sequence, or a compliment of the sequence if the nucleic acid sequence or nucleotide sequence hybridise to one or more than one nucleotide sequence or a compliment of the nucleic acid sequence or nucleotide sequence as defined herein under stringent hybridisation conditions. Sequences are “substantially homologous” “substantially similar” “substantially identical” when at least about 70%, or between 70 to 100%, or any amount therebetween, for example 70, 72, 74, 76, 78, 80, 82, 84, 86, 88, 90, 92, 94, 96, 98, 100%, or any amount therebetween, of the nucleotides match over a defined length of the nucleotide sequence providing that such homologous sequences exhibit one or more than one of the properties of the sequence, or the encoded product as described herein.

Many organisms display a bias for use of particular codons to code for insertion of a particular amino acid in a growing peptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. The process of optimizing the nucleotide sequence coding for a heterologously expressed protein may be an important step for improving expression yields. The optimization requirements may include steps to improve the ability of the host to produce the foreign protein.

There are different codon-optimization techniques known in the art for improving, the translational kinetics of translationally inefficient protein coding regions. These techniques mainly rely on identifying the codon usage for a certain host organism. If a certain gene or sequence should be expressed in this organism, the coding sequence of such genes and sequences will then be modified such that one will replace codons of the sequence of interest by more frequently used codons of the host organism.

“Codon optimization” is defined as modifying a nucleic acid sequence for enhanced expression in a host or host cell of interest by replacing at least one, more than one, or a significant number, of codons of the native sequence with codons that may be more frequently or most frequently used in the genes of another organism or species. Various species exhibit particular bias for certain codons of a particular amino acid.

The present disclosure includes synthetic polynucleotide sequences that have been codon optimized for example the sequences have been optimized for human codon usage or plant codon usage. The codon optimized polynucleotide sequences may then be expressed in the host for example plants. More specifically the sequences optimized for human codon usage or plant codon usage may be expressed in plants. Without wishing to be bound by theory, it is believed that the sequences optimized for human codon increases the guanine-cytosine content (GC content) of the sequence and improves expression yields when plants are used as host.

The term “construct”, “vector” or “expression vector”, as used herein, refers to a recombinant nucleic acid for transferring exogenous nucleotide sequences (for example a nucleotide sequences encoding the modified viral structural protein as described herewith) into host cells (e.g. plant cells) and directing expression of the exogenous nucleic acid sequences in the host cells. “Expression cassette” refers to a nucleic acid comprising a nucleotide sequence of interest under the control of, and operably (or operatively) linked to, an appropriate promoter or other regulatory elements for transcription of the nucleic acid of interest in a host cell. As one of skill in the art would appreciate, the expression cassette may comprise a termination (terminator) sequence that is any sequence that is active the host cell (e.g. plant host). For example in plants, the termination sequence may be derived from the RNA-2 genome segment of a bipartite RNA virus, e.g. a comovirus, the termination sequence may be a NOS terminator, or terminator sequence may be obtained from the 3′UTR of the alfalfa plastocyanin gene.

The nucleic acid comprising a nucleotide sequence encoding a modified viral structural protein, as described herein may further comprise sequences that enhance expression of the viral structural protein in the host, portion of the host or host cell. Sequences that enhance expression may include, a 5′ UTR enhancer element, or a plant-derived expression enhancer, in operative association with the nucleic acid encoding the modified viral structural protein. The sequence encoding the modified viral structural protein may also be optimized to increase expression by for example optimizing for human codon usage, increased GC content, or a combination thereof.

By “regulatory region” “regulatory element” or “promoter” it is meant a portion of nucleic acid typically, but not always, upstream of the protein coding region of a gene, which may be comprised of either DNA or RNA, or both DNA and RNA. When a regulatory region is active, and in operative association, or operatively linked, with a nucleotide sequence of interest, this may result in expression of the nucleotide sequence of interest. A regulatory element may be capable of mediating organ specificity, or controlling developmental or temporal gene activation. A “regulatory region” includes promoter elements, core promoter elements exhibiting a basal promoter activity, elements that are inducible in response to an external stimulus, elements that mediate promoter activity such as negative regulatory elements or transcriptional enhancers. “Regulatory region”, as used herein, also includes elements that are active following transcription, for example, regulatory elements that modulate gene expression such as translational and transcriptional enhancers, translational and transcriptional repressors, upstream activating sequences, and mRNA instability determinants. Several of these latter elements may be located proximal to the coding region.

In the context of this disclosure, the term “regulatory element” or “regulatory region” typically refers to a sequence of DNA, usually, but not always, upstream (5′) to the coding sequence of a structural gene, which controls the expression of the coding region by providing the recognition for RNA polymerase and/or other factors required for transcription to start at a particular site. However, it is to be understood that other nucleotide sequences, located within introns, or 3′ of the sequence may also contribute to the regulation of expression of a coding region of interest. An example of a regulatory element that provides for the recognition for RNA polymerase or other transcriptional factors to ensure initiation at a particular site is a promoter element. Most, but not all, eukaryotic promoter elements contain a TATA box, a conserved nucleic acid sequence comprised of adenosine and thymidine nucleotide base pairs usually situated approximately 25 base pairs upstream of a transcriptional start site. A promoter element may comprise a basal promoter element, responsible for the initiation of transcription, as well as other regulatory elements that modify gene expression.

There are several types of regulatory regions, including those that are developmentally regulated, inducible or constitutive. A regulatory region that is developmentally regulated, or controls the differential expression of a gene under its control, is activated within certain organs or tissues of an organ at specific times during the development of that organ or tissue. However, some regulatory regions that are developmentally regulated may preferentially be active within certain organs or tissues at specific developmental stages, they may also be active in a developmentally regulated manner, or at a basal level in other organs or tissues within the plant as well. Examples of tissue-specific regulatory regions, for example see-specific a regulatory region, include the napin promoter, and the cruciferin promoter (Rask et al., 1998, J. Plant Physiol. 152: 595-599; Bilodeau et al., 1994, Plant Cell 14: 125-130). An example of a leaf-specific promoter includes the plastocyanin promoter (see U.S. Pat. No. 7,125,978, which is incorporated herein by reference).

An inducible regulatory region is one that is capable of directly or indirectly activating transcription of one or more DNA sequences or genes in response to an inducer. In the absence of an inducer the DNA sequences or genes will not be transcribed. Typically the protein factor that binds specifically to an inducible regulatory region to activate transcription may be present in an inactive form, which is then directly or indirectly converted to the active form by the inducer. However, the protein factor may also be absent. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. A plant cell containing an inducible regulatory region may be exposed to an inducer by externally applying the inducer to the cell or plant such as by spraying, watering, heating or similar methods. Inducible regulatory elements may be derived from either plant or non-plant genes (e.g. Gatz, C. and Lenk, I. R. P., 1998, Trends Plant Sci. 3, 352-358). Examples, of potential inducible promoters include, but not limited to, tetracycline-inducible promoter (Gatz, C., 1997, Ann. Rev. Plant Physiol. Plant Mol. Biol. 48, 89-108), steroid inducible promoter (Aoyama, T. and Chua, N. H., 1997, Plant J. 2, 397-404) and ethanol-inducible promoter (Salter, M. G., et al, 1998, Plant Journal 16, 127-132; Caddick, M. X., et al, 1998, Nature Biotech. 16, 177-180) cytokinin inducible IB6 and CKI1 genes (Brandstatter, I. and Kieber, J. J., 1998, Plant Cell 10, 1009-1019; Kakimoto, T., 1996, Science 274, 982-985) and the auxin inducible element, DR5 (Ulmasov, T., et al., 1997, Plant Cell 9, 1963-1971).

A constitutive regulatory region directs the expression of a gene throughout the various parts of a plant and continuously throughout plant development. Examples of known constitutive regulatory elements include promoters associated with the CaMV 35S transcript. (p 35S; Odell et al., 1985, Nature, 313: 810-812; which is incorporated herein by reference), the rice actin 1 (Zhang et al, 1991, Plant Cell, 3: 1155-1165), actin 2 (An et al., 1996, Plant J., 10: 107-121), or tms 2 (U.S. Pat. No. 5,428,147), and triosephosphate isomerase 1 (Xu et. al., 1994, Plant Physiol. 106: 459-467) genes, the maize ubiquitin 1 gene (Cornejo et al, 1993, Plant Mol. Biol. 29: 637-646), the Arabidopsis ubiquitin 1 and 6 genes (Holtorf et al, 1995, Plant Mol. Biol. 29: 637-646), the tobacco translational initiation factor 4A gene (Mandel et al, 1995 Plant Mol. Biol. 29: 995-1004), the Cassava Vein Mosaic Virus promoter, pCAS, (Verdaguer et al., 1996); the promoter of the small subunit of ribulose biphosphate carboxylase, pRbcS: (Outchkourov et al., 2003), the pUbi (for monocots and dicots).

The term “constitutive” as used herein does not necessarily indicate that a nucleotide sequence under control of the constitutive regulatory region is expressed at the same level in all cell types, but that the sequence is expressed in a wide range of cell types even though variation in abundance is often observed.

One or more of the genetic constructs of the present disclosure may also include further enhancers, either translation or transcription enhancers, as may be required. Enhancers may be located 5′ or 3′ to the sequence being transcribed. Enhancer regions are well known to persons skilled in the art, and may include an ATG initiation codon, adjacent sequences or the like. The initiation codon, if present, may be in phase with the reading frame (“in frame”) of the coding sequence to provide for correct translation of the transcribed sequence.

The term “5′UTR” or “5′ untranslated region”, “5′ leader sequence” or “5′ UTR enhancer element” refers to regions of an mRNA that are not translated. The 5′UTR typically begins at the transcription start site and ends just before the translation initiation site or start codon of the coding region. The 5′ UTR may modulate the stability and/or translation of an mRNA transcript.

The term “plant-derived expression enhancer”, as used herein, refers to a nucleotide sequence obtained from a plant, the nucleotide sequence encoding a 5′UTR. Examples of a plant derived expression enhancer are described in U.S. Provisional Patent Application No. 62/643,053 (Filed Mar. 14, 2018) and International Application No. PCT/CA2019/050319 (Filed Mar. 14, 2019); which are incorporated herein by reference) or in Diamos A. G. et al. (2016, Front Plt Sci. 7:1-15; which is incorporated herein by reference). The plant-derived expression enhancer may be selected from nbEPI42, nbSNS46, nbCSY65, nbHEL40, nbSEP44, nbMT78, nbATL75, nbDJ46, nbCHP79, nbEN42, atHSP69, atGRP62, atPK65, atRP46, nb30S72, nbGT61, nbPV55, nbPPI43, nbPM64 and nbH2A86 as described in U.S. 62/643,053 and PCT/CA2019/050319. The plant derived expression enhancer may be used within a plant expression system comprising a regulatory region that is operatively linked with the plant-derived expression enhancer sequence and a nucleotide sequence of interest, for example a nucleotide sequence encoding a modified S protein.

Stability and/or translation efficiency of an RNA may further be improved by the inclusion of a 3′ untranslated region (3′UTR). The one or more genetic constructs of the present description may therefore further comprise a 3′ UTR.

A 3′ untranslated region may contain a polyadenylation signal and any other regulatory signals capable of effecting mRNA processing or gene expression. The polyadenylation signal is usually characterized by effecting the addition of polyadenylic acid tracks to the 3′ end of the mRNA precursor. Polyadenylation signals are commonly recognized by the presence of homology to the canonical form 5′ AATAAA-3′ although variations are not uncommon. Non-limiting examples of suitable 3′ regions are the 3′ transcribed non-translated regions containing a polyadenylation signal of Agrobacterium tumor inducing (Ti) plasmid genes, such as the nopaline synthase (Nos gene) and plant genes such as the soybean storage protein genes, the small subunit of the ribulose-1, 5-bisphosphate carboxylase gene (ssRUBISCO; U.S. Pat. No. 4,962,028; which is incorporated herein by reference), the promoter used in regulating plastocyanin expression, described in U.S. Pat. No. 7,125,978 (which is incorporated herein by reference), 3′ UTR derived from a Arracacha virus B isolate gene (AvB) (SEQ ID NO: 40), 3′UTR derived from Beet necrotic yellow vein virus (trBNYVV) (SEQ ID NO: 41), 3′UTR derived from Southern bean mosaic virus (SBMV) (SEQ ID NO: 42), 3′UTR derived from Turnip ringspot virus (TuRSV) (SEQ ID NO: 43), 3′ UTR derived from Cowpea Mosaic Virus (CPMV) (SEQ ID NO: 44), 3′UTR derived from Broad bean true mosaic virus (BBTMV) (SEQ ID NO: 45) or 3′UTR derived from Ourmia melon virus (trOUMV) (SEQ ID NO: 46). The 3′UTR might be used in conjunction with 5′UTR derived from heterologous sequences to modulate expression levels.

It is therefore provided a “construct”, “vector”, “expression vector” or “expression cassette” that comprises a nucleic acid comprising a nucleotide sequence of interest (such as a modified viral structural protein) under the control of, and operably (or operatively) linked to a 3′UTR. Furthermore, the nucleic acid may comprise a 3′UTR operably (or operatively) linked to a nucleotide sequence of interest (such as a modified viral structural protein).

The modified viral structural protein may be targeted to any intracellular or extracellular space, organelle or tissue of a host of host cell such as plant or plant cell as desired. In order to localize the expressed protein to a particular location, the nucleic acid encoding the protein may be linked to a nucleic acid sequence encoding a signal peptide or leader sequence. A signal peptide may alternately be referred to as a transit peptide, signal sequence, leader sequence, targeting signal, localization signal, localization sequence, transit peptide, or leader peptide.

The one or more than one modified genetic constructs of the present description may be expressed in any suitable host or host cell that is transformed by the nucleic acids, or nucleotide sequence, or constructs, or vectors of the present disclosure. The host or host cell may be from any source including plants, fungi, bacteria, insect and animals for example mammals. Therefore the host or host cell may be selected from a plant or plant cell, a fungi or a fungi cell, a bacteria or bacteria cell, an insect or an insect cell, and animal or an animal cell. The mammal or animal may not be a human. In a preferred embodiment the host or host cell is a plant, portion of a plant or plant cell.

The term “plant”, “portion of a plant”, “plant portion”, “plant matter”, “plant biomass”, “plant material”, plant extract”, or “plant leaves”, as used herein, may comprise an entire plant, tissue, cells, or any fraction thereof, intracellular plant components, extracellular plant components, liquid or solid extracts of plants, or a combination thereof, that are capable of providing the transcriptional, translational, and post-translational machinery for expression of one or more than one nucleic acids described herein, and/or from which an expressed protein or VLP may be extracted and purified. Plants may include, but are not limited to, herbaceous plants. The herbaceous plants may be annuals, biennials or perennials plants. Plants may further include, but are not limited to agricultural crops including for example canola, Brassica spp., maize, Nicotiana spp., (tobacco) for example, Nicotiana benthamiana, Nicotiana rustica, Nicotiana, tabacum, Nicotiana alata, Arabidopsis thaliana, alfalfa, potato, sweet potato (Ipomoea batatus), ginseng, pea, oat, rice, soybean, wheat, barley, sunflower, cotton, corn, rye (Secale cereale), Sorghum (Sorghum bicolor, Sorghum vulgare), safflower (Carthamus tinctorius).

The term “plant portion”, as used herein, refers to any part of the plant including but not limited to leaves, stem, root, flowers, fruits, a plant cell obtained from leaves, stem, root, flowers, fruits, a plant extract obtained from leaves, stem, root, flowers, fruits, or a combination thereof. In one embodiment the plant portion refers to the areal portion of a plant such as for example leaves, stem, flowers and fruits. The term “plant extract”, as used herein, refers to a plant-derived product that is obtained following treating a plant, a portion of a plant, a plant cell, or a combination thereof, physically (for example by freezing followed by extraction in a suitable buffer), mechanically (for example by grinding or homogenizing the plant or portion of the plant followed by extraction in a suitable buffer), enzymatically (for example using cell wall degrading enzymes), chemically (for example using one or more chelators or buffers), or a combination thereof. A plant extract may be further processed to remove undesired plant components for example cell wall debris. A plant extract may be obtained to assist in the recovery of one or more components from the plant, portion of the plant or plant cell, for example a protein (including protein complexes, protein surprastructures and/or VLPs), a nucleic acid, a lipid, a carbohydrate, or a combination thereof from the plant, portion of the plant, or plant cell. If the plant extract comprises proteins, then it may be referred to as a protein extract. A protein extract may be a crude plant extract, a partially purified plant or protein extract, or a purified product, that comprises one or more proteins, protein complexes such for example protein trimers, protein suprastructures, and/or VLPs, from the plant tissue. If desired a protein extract, or a plant extract, may be partially purified using techniques known to one of skill in the art, for example, the extract may be subjected to salt or pH precipitation, centrifugation, gradient density centrifugation, filtration, chromatography, for example, size exclusion chromatography, ion exchange chromatography, affinity chromatography, or a combination thereof. A protein extract may also be purified, using techniques that are known to one of skill in the art.

The constructs of the present disclosure can be introduced into plant cells using Ti plasmids, Ri plasmids, plant virus vectors, direct DNA transformation, micro-injection, electroporation, etc. For reviews of such techniques see for example Weissbach and Weissbach, Methods for Plant Molecular Biology, Academy Press, New York VIII, pp. 421-463 (1988); Geierson and Corey, Plant Molecular Biology, 2d Ed. (1988); and Miki and Iyer, Fundamentals of Gene Transfer in Plants. In Plant Metabolism, 2d Ed. DT. Dennis, DH Turpin, DD Lefebvre, DB Layzell (eds), Addison Wesly, Langmans Ltd. London, pp. 561-579 (1997). Other methods include direct DNA uptake, the use of liposomes, electroporation, for example using protoplasts, micro-injection, microprojectiles or whiskers, and vacuum infiltration. See, for example, Bilang, et al. (Gene 100: 247-250 (1991), Scheid et al. (Mol. Gen. Genet. 228: 104-112, 1991), Guerche et al. (Plant Science 52: 111-116, 1987), Neuhause et al. (Theor. Appl Genet. 75: 30-36, 1987), Klein et al., Nature 327: 70-73 (1987); Howell et al. (Science 208: 1265, 1980), Horsch et al. (Science 227: 1229-1231, 1985), DeBlock et al., Plant Physiology 91: 694-701, 1989), Methods for Plant Molecular Biology (Weissbach and Weissbach, eds., Academic Press Inc., 1988), Methods in Plant Molecular Biology (Schuler and Zielinski, eds., Academic Press Inc., 1989), Liu and Lomonossoff (J Virol Meth, 105:343-348, 2002), U.S. Pat. Nos. 4,945,050; 5,036,006; and 5,100,792, U.S. patent application Ser. No. 08/438,666, filed May 10, 1995, and Ser. No. 07/951,715, filed Sep. 25, 1992, (all of which are hereby incorporated by reference).

As described below, transient expression methods may be used to express the constructs of the present disclosure (see Liu and Lomonossoff, 2002, Journal of Virological Methods, 105:343-348; which is incorporated herein by reference). Alternatively, a vacuum-based transient expression method, as described by Kapila et al., 1997, which is incorporated herein by reference) may be used. These methods may include, for example, but are not limited to, a method of Agro-inoculation or Agroinfiltration, syringe infiltration, however, other transient methods may also be used as noted above. With Agro-inoculation, Agroinfiltration, or syringe infiltration, a mixture of Agrobacteria comprising the desired nucleic acid enter the intercellular spaces of a tissue, for example the leaves, aerial portion of the plant (including stem, leaves and flower), other portion of the plant (stem, root, flower), or the whole plant. After crossing the epidermis the Agrobacteria infect and transfer t-DNA copies into the cells. The t-DNA is episomally transcribed and the mRNA translated, leading to the production of the protein of interest in infected cells, however, the passage of t-DNA inside the nucleus is transient.

To aid in identification of transformed plant cells, the constructs of this disclosure may be further manipulated to include plant selectable markers. Useful selectable markers include enzymes that provide for resistance to chemicals such as an antibiotic for example, gentamycin, hygromycin, kanamycin, or herbicides such as phosphinothrycin, glyphosate, chlorosulfuron, and the like. Similarly, enzymes providing for production of a compound identifiable by colour change such as GUS (beta-glucuronidase), or luminescence, such as luciferase or GFP, may be used.

Also considered part of this disclosure are transgenic plants, plant cells or seeds containing the gene construct of the present disclosure that may be used as a platform plant suitable for transient protein expression described herein. Methods of regenerating whole plants from plant cells are also known in the art (for example see Guerineau and Mullineaux (1993, Plant transformation and expression vectors. In: Plant Molecular Biology Labfax (Croy RRD ed) Oxford, BIOS Scientific Publishers, pp 121-148). In general, transformed plant cells are cultured in an appropriate medium, which may contain selective agents such as antibiotics, where selectable markers are used to facilitate identification of transformed plant cells. Once callus forms, shoot formation can be encouraged by employing the appropriate plant hormones in accordance with known methods and the shoots transferred to rooting medium for regeneration of plants. The plants may then be used to establish repetitive generations, either from seeds or using vegetative propagation techniques. Transgenic plants can also be generated without using tissue culture. Methods for stable transformation, and regeneration of these organisms are established in the art and known to one of skill in the art. Available techniques are reviewed in Vasil et al. (Cell Culture and Somatic Cell Genetics of Plants, Vol I, Il and III, Laboratory Procedures and Their Applications, Academic Press, 1984), and Weissbach and Weissbach (Methods for Plant Molecular Biology, Academic Press, 1989). The method of obtaining transformed and regenerated plants is not critical to the present disclosure.

If plants, plant portions or plant cells are to be transformed or co-transformed by two or more nucleic acid constructs, the nucleic acid construct may be introduced into the Agrobacterium in a single transfection event so that the nucleic acids are pooled, and the bacterial cells transfected. Alternatively, the constructs may be introduced serially. In this case, a first construct is introduced into the Agrobacterium as described, the cells are grown under selective conditions (e.g. in the presence of an antibiotic) where only the singly transformed bacteria can grow. Following this first selection step, a second nucleic acid construct is introduced into the Agrobacterium as described, and the cells are grown under double-selective conditions, where only the double-transformed bacteria can grow. The double-transformed bacteria may then be used to transform a plant, portion of the plant or plant cell as described herein, or may be subjected to a further transformation step to accommodate a third nucleic acid construct.

Alternatively, if plants, plant portions, or plant cells are to be transformed or co-transformed by two or more nucleic acid constructs, the nucleic acid construct may be introduced into the plant by co-infiltrating a mixture of Agrobacterium cells with the plant, plant portion, or plant cell, each Agrobacterium cell may comprise one or more constructs to be introduced within the plant. In order to vary the relative expression levels within the plant, plant portion or plant cell, of a nucleotide sequence of interest within a construct, during the step of infiltration, the concentration of the various Agrobacteria populations comprising the desired constructs may be varied.

The modified viral surface protein or VLP comprising modified viral surface protein as described herewith, may be used to elicit an immune response in a subject.

An “immune response” generally refers to a response of the adaptive immune system of a subject. The adaptive immune system generally comprises a humoral response, and a cell-mediated response. The humoral response is the aspect of immunity that is mediated by secreted antibodies, produced in the cells of the B lymphocyte lineage (B cell). Secreted antibodies bind to antigens on the surfaces of invading microbes (such as viruses or bacteria), which flags them for destruction. Humoral immunity is used generally to refer to antibody production and the processes that accompany it, as well as the effector functions of antibodies, including Th2 cell activation and cytokine production, memory cell generation, opsonin promotion of phagocytosis, pathogen elimination and the like. The terms “modulate” or “modulation” or the like refer to an increase or decrease in a particular response or parameter, as determined by any of several assays generally known or used, some of which are exemplified herein.

A cell-mediated response is an immune response that does not involve antibodies but rather involves the activation of macrophages, natural killer cells (NK), antigen-specific cytotoxic T-lymphocytes, and the release of various cytokines in response to an antigen. Cell-mediated immunity is used generally to refer to some Th cell activation, Tc cell activation and T-cell mediated responses. Cell mediated immunity may be of particular importance in responding to viral infections.

For example, the induction of antigen specific CD8 positive T lymphocytes may be measured using an ELISPOT assay; stimulation of CD4 positive T-lymphocytes may be measured using a proliferation assay. Anti-Coronavirus antibody titers may be quantified using an ELISA assay; isotypes of antigen-specific or cross-reactive antibodies may also be measured using anti-isotype antibodies (e.g. anti-IgG, IgA, IgE or IgM). Methods and techniques for performing such assays are well-known in the art.

Cytokine presence or levels may also be quantified. For example a T-helper cell response (Th1/Th2) will be characterized by the measurement of IFN-γ and TL-4 secreting cells using by ELISA (e.g. BD Biosciences OptEIA kits). Peripheral blood mononuclear cells (PBMC) or splenocytes obtained from a subject may be cultured, and the supernatant analyzed. T lymphocytes may also be quantified by fluorescence-activated cell sorting (FACS), using marker specific fluorescent labels and methods as are known in the art.

A microneutralization assay may also be conducted to characterize an immune response in a subject, see for example the methods of Rowe et al., 1973. Virus neutralization titers may be quantified in a number of ways, including: enumeration of lysis plaques (plaque assay) following crystal violent fixation/coloration of cells; microscopic observation of cell lysis in in vitro culture; and 2) ELISA and spectrophotometric detection of Coronavirus.

The term “epitope” or “epitopes”, as used herein, refers to a structural part of an antigen to which an antibody specifically binds.

A method of producing an antibody or antibody fragment is provided, the method comprises administering the modified viral structural protein, a trimer or trimeric modified viral structural protein or VLP comprising the modified viral structural protein as described herewith to a subject, or a host animal, thereby producing the antibody or the antibody fragment. Antibodies or the antibody fragments produced by the method are also provided.

The present disclosure therefore also provides the use of a viral structural protein or VLP comprising the modified viral structural protein, as described herein, for inducing immunity to a Coronavirus infection in a subject. Also disclosed herein is an antibody or antibody fragment, prepared by administering the modified viral structural protein or VLP comprising the modified viral structural protein, to a subject or a host animal.

Further provided is a composition comprising an effective dose of modified viral structural protein or VLP comprising the modified viral structural protein, as described herein, and a pharmaceutically acceptable carrier, adjuvant, vehicle, or excipient, for inducing an immune response in a subject. Also provided is a vaccine for inducing an immune response again Coronavirus in a subject, wherein the vaccine comprises an effective dose of the modified viral structural protein or VLP comprising the modified viral structural protein.

Further provided is a composition that may comprise a mixture of VLPs provided that at least one of the VLPs within the composition comprises modified coronavirus S protein as described herein. For example, each coronavirus S protein including one or more than one modified S protein, from each of one or more than one Coronavirus family, sub-group, type, subtype, lineage or strain may be expressed and the corresponding VLPs purified. Virus like particles obtained from two or more than two Coronavirus families, sub-groups, types, subtypes, lineages or strains (for example, two, three, four, five, six, seven, eight, nine, 10 or more Coronavirus families, sub-groups, types, subtypes, lineages or strains) may be combined as desired to produce a mixture of VLPs, provided that one or more than one VLP in the mixture of VLPs comprises a modified S protein as described herein. The VLPs may be combined or produced in a desired ratio, for example about equivalent ratios, or may be combined in such a manner that one Coronavirus family, sub-group, type, subtype, lineage or strain comprises the majority of the VLPs in the composition. It is further provided a composition of VLPs comprising one or more than one modified S protein with ectodomain and/or TM or portion of a TM derived from each of one or more than one Coronavirus family, sub-group, type, subtype, lineage or strain, such that a mixture of different modified S protein as provided for in this disclosure may be present in any individual VLP of the composition.

The composition or vaccine may comprise VLP comprising the modified viral structural protein, such as the modified S protein from one type of Coronavirus family, sub-group, type, subtype, lineage or strain, or the composition or vaccine may comprise multiple VLP types, wherein each VLP type comprises modified S protein, wherein the modified S proteins in the same VLP are derived from one type of Coronavirus family, sub-group, type, subtype, lineage or strain i.e. the composition or vaccine may comprise a mixture of different Coronavirus VLP, wherein each VLP may comprise a modified S protein from the same Coronavirus family, sub-group, type, subtype, lineage or strain. For example the composition or vaccine may comprise a first VLP comprising a first modified S protein from a first Coronavirus family, sub-group, type, subtype, lineage or strain and a second VLP comprising a second modified S protein from a second Coronavirus family, sub-group, type, subtype, lineage or strain. Furthermore the composition may also comprise a third VLP comprising a third modified S protein from a third Coronavirus family, sub-group, type, subtype, lineage or strain and/or the composition or vaccine may comprise a fourth VLP comprising a fourth modified S protein from a fourth Coronavirus family, sub-group, type, subtype, lineage or strain. Accordingly, the description also provides compositions or vaccines that are monovalent (univalent), or multivalent (polyvalent). The monovalent composition or vaccine may immunize a subject against a single type of Coronavirus strain, whereas the multivalent composition or vaccine may immunize a subject against more than one Coronavirus strain. For example, the composition or vaccine may be a bivalent composition or vaccine, which upon administration, may immunize a subject against two different types of Coronavirus families, sub-groups, types, subtypes, lineages or strains. Furthermore, the composition or vaccine may be a trivalent composition, or the vaccine or composition may be a tetravalent or quadrivalent composition or vaccine.

Furthermore, the multivalent composition may comprise VLP comprising one or more than one modified S proteins with different HA cytoplasmic tails. For example, the multivalent composition may comprise a VLP or plurality of VLPs comprising two or more modified S proteins, each comprising a S protein ectodomain, a S protein transmembrane domain, and a cytoplasmic tail derived from HA from an influenza H1, H3, H5, H6, H7, H9 or B strain. Non-limiting examples of influenza strains are for example H1 California/7/2009, H3 A/Minnesota/41/2019, H5 A/Indonesia/5/05, H6 A/Teal/Hong Kong/W312/97, H7 A/Guangdong/17SF003/2016, H9 A/Hong Kong/1073/99 or B/Washington/02/2019.

The multivalent composition or vaccine with multiple type VLPs may further comprise a pharmaceutically acceptable carrier, adjuvant, vehicle, or excipient, for inducing an immune response in a subject.

Adjuvant systems to enhance a subject's immune response to a vaccine antigen are well known and may be used in conjunction with the vaccine or pharmaceutical composition as described herewith. There are many types of adjuvants that may be used. Common adjuvants for human use are aluminum hydroxide, aluminum phosphate and calcium phosphate. There are also a number of adjuvants based on oil emulsions (oil in water or water in oil emulsions such as Freund's incomplete adjuvant (FIA), Montanide™, Adjuvant 65, and Lipovant™), products from bacterial (or their synthetic derivatives), endotoxins, fatty acids, paraffinic, or vegetable oils, cholesterols, and aliphatic amines or natural organic compounds such for example squalene. Non-limiting adjuvants that might be used include for example oil-in water emulsions of squalene oil (for example MF-59 or AS03), adjuvant composed of the synthetic TLR4 agonist glucopyranosyl lipid A (GLA) integrated into stable emulsion (SE) (GLA-SE) or CpG 1018 a toll-like receptor (TLR9) agonist adjuvant.

Therefore the vaccine or pharmaceutical composition may comprise one or more than one adjuvant. For example the vaccine or pharmaceutical composition may comprise aluminum hydroxide, aluminum phosphate, calcium phosphate, an oil in water or water in oil emulsions, an emulsion comprising squalene (for example MF-59 or AS03), an emulsion comprising GLA-SE, or CpG 1018 adjuvant.

The pharmaceutical compositions, vaccines or formulations of the present description may be manufactured in a manner that is itself known, e.g., by means of conventional mixing, dissolving, granulating, dragee-making, levigating, emulsifying, encapsulating, entrapping or tableting processes.

The pharmaceutical compositions, vaccines or formulations may be produced by mixing or premixing of any constituent components before administration, for example by manual or mechanically-aided mixing of two or more vaccine suspensions, pharmaceutically acceptable carriers, adjuvants, vehicles, or excipients as a step performed before the final formulation, vaccine, or pharmaceutical composition is administered.

The pharmaceutical compositions, vaccines or formulations may be administered to a subject orally, intradermally, intranasally, intramuscularly, intraperitoneally, intravenously, or subcutaneously.

Injectables can be prepared in conventional forms, either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride, and the like. In addition, if desired, the injectable pharmaceutical compositions may contain minor amounts of nontoxic auxiliary substances, such as wetting agents, pH buffering agents, and the like. Physiologically compatible buffers include, but are not limited to, Hanks's solution, Ringer's solution, or physiological saline buffer. If desired, absorption enhancing preparations (for example, liposomes), may be utilized.

The composition or vaccine may be administered to a subject once (single dose). Furthermore, the vaccine or composition may be administered to a subject multiple times (multi-dose). Therefore the composition, formulation, or vaccine may be administered to a subject in a single dose to illicit an immune response or the composition, formulation, or vaccine may be administered multiple time (multi dosages). For example a dose of the composition or vaccine may be administered 2, 3, 4 or 5 times. Accordingly, the composition or vaccine may be administered to a subject in an initial dose and one or more than one doses may subsequently be administered to the subject. Administration of the doses may be separated in time from each other. For example after the administration of an initial dose, one or more than one subsequent dose may be administered 1 day, 2 days, 3 days, 4 days, 5 days, 6 days, 1 week, 2 weeks, 3 weeks, 1 month, 2 months, 3 months, 4 months, 5 months or 6 months or any time in between from the administration of the initial dose. Furthermore, the composition or vaccine may be administered annually. For example the composition or vaccine may be administered as a seasonal vaccine.

The disclosure further provides the following sequences.

TABLE 4
SEQ ID NOs and Description of Sequences

| SEQ | |
|---|---|
| ID NO: | Description of Sequence |
| 1 | Native SARS-CoV-2 S protein wtTM/CT AA (P0DTC2) |
| 2 | Native SARS-CoV-2 S protein wtTM/CT AA (P0DTC2) without signal peptide (SP) |
| 3 | H5 A/Indonesia/5/05 Hemagglutinin (HA) AA (A5A5L7) |
| 4 | H5 A/Indonesia/5/05 Hemagglutinin (HA) viral cDNA (EF541394.1) |
| 5 | Modified SARS-CoV-2 with H5 A/Indonesia/5/05 (H5i) Hemagglutinin CT AA |
| 6 | H1 California/7/2009Hemagglutinin TM/CT region AA |
| 7 | H2 A/Singapore/1/1957 HA Hemagglutinin TM/CT region AA |
| 8 | H3 A/Minnesota/41/2019 Hemagglutinin TM/CT region AA |
| 9 | H5 A/Indonesia/5/05 Hemagglutinin TM/CT region AA |
| 10 | H6 A/Teal/Hong Kong/W312/97 Hemagglutinin TM/CT region AA |
| 11 | H7 A/Guangdong/17SF003/2016 Hemagglutinin TM/CT region AA |
| 12 | H9 A/Hong Kong/1073/99 Hemagglutinin TM/CT region AA |
| 13 | B/Washington/02/2019 Hemagglutinin TM/CT region AA |
| 14 | Consensus Sequence of C-Terminal Region of Influenza Hemagglutinin TM/CT region |
| 15 | Consensus Sequence of CT Domain of Influenza Hemagglutinin |
| 16 | TM/CT Region of Native SARS-CoV-2 S protein (wtTM/CT) |
| 17 | TM/CT of H5 A/Indonesia/5/05 Hemagglutinin (H5iTM/CT) |
| 18 | TM/CT Region of Modified SARS-CoV-2 S protein with H5i Hemagglutinin CT |
| 19 | TM/CT Region of Modified S protein with H5i Hemagglutinin CT (Variation 1) |
| 20 | (no SP) SARS-CoV-2 S protein GSAS + PP wtTM/CT AA |
| 21 | (no SP) Modified SARS-CoV-2 S protein GSAS + PP with H5i Hemagglutinin CT AA |
| 22 | PDI-SARS-CoV-2 S protein GSAS + PP wtTM/CT-DNA |
| 23 | PDI-SARS-CoV-2 S protein GSAS + PP wtTM/CT-AA (product of construct 8591) |
| 24 | IF(PDI)-CoV(opt2).c |
| 25 | IF(AVB)-CoV(opt2).r |
| 26 | PDI-Modified SARS-CoV-2 S protein GSAS + PP H5iTM/CT-DNA |
| 27 | PDI-Modified SARS-CoV-2 S protein GSAS + PP H5iTM/CT-AA (product of construct 8597) |
| 28 | IF(Avb)-H5I.r |
| 29 | PDI-Modified SARS-CoV-2 S protein GSAS + PP wtTM/H5iCT-DNA |
| 30 | PDI-Modified SARS-CoV-2 S protein GSAS + PP wtTM/H5iCT-AA (product of construct 8671) |
| 31 | Cloning vector 8501 from left to right T-DNA |
| 32 | Construct 8586 from 2X35S(+C) prom to NOS term |
| 33 | Cloning vector 8500 from left to right T-DNA] |
| 34 | Construct 8589 from 2X35S(+C) prom to NOS term |
| 35 | Cloning vector 8716 from left to right T-DNA |
| 36 | Construct 8591 from 2X35S(+C) prom to NOS term |
| 37 | C-Terminal Region of Modified S protein with H5i Hemagglutinin CT (alternative 2) |
| 38 | C-Terminal Region of Modified S protein with H5i Hemagglutinin CT (alternative 3) |
| 39 | C-Terminal Region of Modified S protein with H5i Hemagglutinin CT (alternative 4) |
| 40 | 3′UTR AvB (Arracacha Virus B Isolate) |
| 41 | 3′UTR trBNYVV (Beet necrotic yellow vein virus) |
| 42 | 3′ UTR SBMV (Southern bean mosaic virus) |
| 43 | 3′ UTR TuRSV (Turnip ringspot virus) |
| 44 | 3′ UTR CPMV (Cowpea Mosaic virus) |
| 45 | 3′ BBTMV (Broad bean true mosaic virus) |
| 46 | 3′ UTR trOUMV (Ourmia melon virus) |
| | Modified S Protein with Substitutions |
| 47 | PDI-S Protein GSAS-2P (product of construct 8671) |
| 48 | PDI-S Protein GSAS-4P (product of construct 8953) |
| 49 | PDI-S Protein GSAS-6P (product of construct 8940) |
| 50 | PDI-S Protein GSAS-2P-923 (product of construct 8933) |
| 51 | PDI-S Protein GSAS-4P-923 (product of construct 8960) |
| 52 | PDI-S Protein GSAS-6P-923 (product of construct 8947) |
| | Modified S with CT from different HA strains |
| 53 | PDI-S-protein + H1 Cal (product of construct 7390) |
| 54 | PDI-S-protein + H3 Minn (product of construct 7391) |
| 55 | PDI-S-protein + H6 HK (product of construct 7392) |
| 56 | PDI-S-protein + H7 Guangdong (product of construct 7393) |
| 57 | PDI-S-protein + H9 HK (product of construct 7394) |
| 58 | PDI-S-protein + B/Wash (product of construct 7395) |
| | Modified S Protein C-terminal variations |
| 59 | PDI-Modified S protein with H5i Hemagglutinin CT (V1) (product of construct 8980) |
| 60 | PDI-Modified S protein with H5i Hemagglutinin CT (V2) (product of construct 8981) |
| 61 | PDI-Modified S protein with H5i Hemagglutinin CT (V3) (product of construct 8982) |
| 62 | PDI-Modified S protein with H5i Hemagglutinin CT (V4) (product of construct 8983) |
| | N-terminal region |
| 63 | N-terminal region of native SARS-CoV-2 protein (including native signal peptide) |
| 64 | TM/CT Region of Modified SARS-CoV-2 S protein with intervening peptide sequence Xₙ |
| | Modified S Protein with Substitutions (DNA) |
| 65 | PDI-S Protein GSAS + 4P-DNA |
| 66 | PDI-S Protein GSAS + 6P-DNA |
| 67 | PDI-S Protein GSAS + 2P + L923F-DNA |
| 68 | PDI-S Protein GSAS + 4P + L923F-DNA |
| 69 | PDI-S Protein GSAS + 6P + L923F-DNA |
| | Modified S Protein C-terminal variations (DNA) |
| 70 | PDI-Modified S protein with H5i Hemagglutinin CT (V1) DNA |
| 71 | PDI-Modified S protein with H5i Hemagglutinin CT (V2) DNA |
| 72 | PDI-Modified S protein with H5i Hemagglutinin CT (V3) DNA |
| 73 | PDI-Modified S protein with H5i Hemagglutinin CT (V4) DNA |
| | Modified S with CT from different HA strains (DNA) and Primers |
| 74 | PDI-S-protein + H1 Cal DNA |
| 75 | PDI-S-protein + H3 Minn DNA |
| 76 | PDI-S-protein + H6 HK DNA |
| 77 | PDI-S-protein + H7 Guangdong DNA |
| 78 | PDI-S-protein + H9 HK DNA |
| 79 | PDI-S-protein + B/Wash DNA |
| 80 | IF-H1HawaiiCT.r |
| 81 | IF-H3MinnesotaCT.r |
| 82 | IF-HongKongCT.r |
| 83 | IF-GuangdongCT.r |
| 84 | IF-H9HKCT.r |
| 85 | IF-BWashCT.r |
| | Modified SARS-COV-1 S Protein (DNA) and Primers |
| 86 | IF(nbHEL40)-PDI.c |
| 87 | IF(AvB + wtCT).r |
| 88 | PDI-SARS-COV-1 wtTMCT-DNA |
| 89 | PDI-SARS-COV-1 H5iTMCT-DNA |
| 90 | PDI-SARS-COV-1 H5iCT-DNA |
| 91 | PDI-SARS-COV-1 H5iCT(V4)-DNA |
| 92 | PDI-SARS-COV-1 H1cCT-DNA |
| | Modified SARS-COV-1 S Protein (AA) |
| 93 | PDI-SARS-COV-1 wtTMCT-AA |
| 94 | PDI-SARS-COV-1 H5iTMCT-AA |
| 95 | PDI-SARS-COV-1 H5iCT-AA |
| 96 | PDI-SARS-COV-1 H5iCT(V4)-AA |
| 97 | PDI-SARS-COV-1 H1cCT-AA |
| | Modified MERS S Protein (DNA) and Primers |
| 98 | IF(AvB + wtCT-MERS).r |
| 99 | IF(H1cCT-wtTM).r |
| 100 | IF(H5ITMCT).r |
| 101 | PDI-MERS-wtTMCT-DNA |
| 102 | PDI-MERS-H5iTMCT-DNA |
| 103 | PDI-MERS-H5iCT-DNA |
| 104 | PDI-MERS-H5iCT(V4)-DNA |
| 105 | PDI-MERS-H1cCT-DNA |
| | Modified SARS-COV-1 S Protein (AA) |
| 106 | PDI-MERS-wtTMCT-AA |
| 107 | PDI-MERS-H5iTMCT-AA |
| 108 | PDI-MERS-H5iCT-AA |
| 109 | PDI-MERS-H5iCT(V4)-AA |
| 110 | PDI-MERS-H1cCT-AA |
| | Other Sequences |
| 111 | Cloning vector 7147 from left to right T-DNA |
| 112 | Native SARS-CoV-1 S protein wtTM/CT AA (P59594) |
| 113 | Native MERS S protein wtTM/CT AA (AFY13307) |
| 114 | Native SARS-CoV-1 S protein wtTM/CT AA (P59594) without signal peptide |
| 115 | Native MERS S protein wtTM/CT AA (AFY13307) without signal peptide |
| 116 | TMCT region of modified PDI-SARS-COV-1 wtTMCT-AA |
| 117 | TMCT region of modified PDI-SARS-COV-1 H5iTMCT-AA |
| 118 | TMCT region of modified PDI-SARS-COV-1 H5iCT-AA |
| 119 | TMCT region of modified PDI-SARS-COV-1 H5iCT(V4)-AA |
| 120 | TMCT region of modified PDI-SARS-COV-1 H1cCT-AA |
| 121 | TMCT region of modified PDI-MERS-wtTMCT-AA |
| 122 | TMCT region of modified PDI-MERS-H5iTMCT-AA |
| 123 | TMCT region of modified PDI-MERS-H5iCT-AA |
| 124 | TMCT region of modified PDI-MERS-H5iCT(V4)-AA |
| 125 | TMCT region of modified PDI-MERS-H1cCT-AA |
| 126 | TMCT region of modified PDI-S-protein + H1 Cal |
| 127 | TMCT region of modified PDI-S-protein + H3 Minn |
| 128 | TMCT region of modified PDI-S-protein + H6 HK |
| 129 | TMCT region of modified PDI-S-protein + H7 Guangdong |
| 130 | TMCT region of modified PDI-S-protein + H9 HK |
| 131 | TMCT region of modified PDI-S-protein + B/Wash |
| 132 | Consensus Sequence of TM Domain of Coronavirus S-protein |
| 133 | Consensus Sequence of TM Domain of Coronavirus S-protein |
| 134 | TM/CT Region of Modified SARS-CoV-1 S protein with intervening peptide sequence Xₙ |
| 135 | TM/CT Region of Modified MERS S protein with intervening peptide sequence Xₙ |
| | Modified OC43-CoV S Protein (DNA) and Primers |
| 136 | IF(AvB + wtCT-OC43).r |
| 137 | PDI-OC43-wtTMCT-DNA |
| 138 | PDI-OC43-H5iTMCT-DNA |
| 139 | PDI-OC43-H5iCT-DNA |
| 140 | PDI-OC43-H5iCT(V4)-DNA |
| 141 | PDI-OC43-H1cCT-DNA |
| | Modified OC43-CoV S Protein (AA) |
| 142 | PDI-OC43-wtTMCT-AA |
| 143 | PDI-OC43-H5iTMCT-AA |
| 144 | PDI-OC43-H5iCT-AA |
| 145 | PDI-OC43-H5iCT(V4)-AA |
| 146 | PDI-OC43-HlcCT-AA |
| | Modified 229E-CoV S Protein (DNA) and Primers |
| 147 | IF(CoV229EwtCT).r |
| 148 | PDI-229E -wtTMCT-DNA |
| 149 | PDI-229E -H5iTMCT-DNA |
| 150 | PDI-229E -H5iCT-DNA |
| 151 | PDI-229E -H5iCT(V4)-DNA |
| 152 | PDI-229E -H1cCT-DNA |
| | Modified 229E-CoV S Protein (AA) |
| 153 | PDI-229E -wtTMCT-AA |
| 154 | PDI-229E -H5iTMCT-AA |
| 155 | PDI-229E -H5iCT-AA |
| 156 | PDI-229E -H5iCT(V4)-AA |
| 157 | PDI-229E-H1cCT-AA |
| | Other Sequences |
| 158 | Native OC43-CoV S protein wtTM/CT AA (AVR40344) |
| 159 | Native 229E S protein wtTM/CT AA (P15423) |
| 160 | Native OC43-CoV S protein wtTM/CT AA (AVR40344) without signal peptide |
| 161 | Native 229E S protein wtTM/CT AA (P15423) without signal peptide |
| 162 | TMCT region of modified PDI-OC43-COV wtTMCT-AA |
| 163 | TMCT region of modified PDI-OC43-COV H5iTMCT-AA |
| 164 | TMCT region of modified PDI-OC43-COV H5iCT-AA |
| 165 | TMCT region of modified PDI-OC43-COV H5iCT(V4)-AA |
| 166 | TMCT region of modified PDI-OC43-COV HIcCT-AA |
| 167 | TMCT region of modified PDI-229E-wtTMCT-AA |
| 168 | TMCT region of modified PDI-229E-H5iTMCT-AA |
| 169 | TMCT region of modified PDI-229E-H5iCT-AA |
| 170 | TMCT region of modified PDI-229E-H5iCT(V4)-AA |
| 171 | TMCT region of modified PDI-229E-H1cCT-AA |
| 172 | TM/CT Region of Modified OC43-CoV S protein with intervening peptide sequence Xₙ |
| 173 | TM/CT Region of Modified 229E-CoV S protein with intervening peptide sequence Xₙ |

The present invention will be further illustrated in the following examples.

### EXAMPLES

### Example 1: Preparation of Modified Structural Protein Constructs

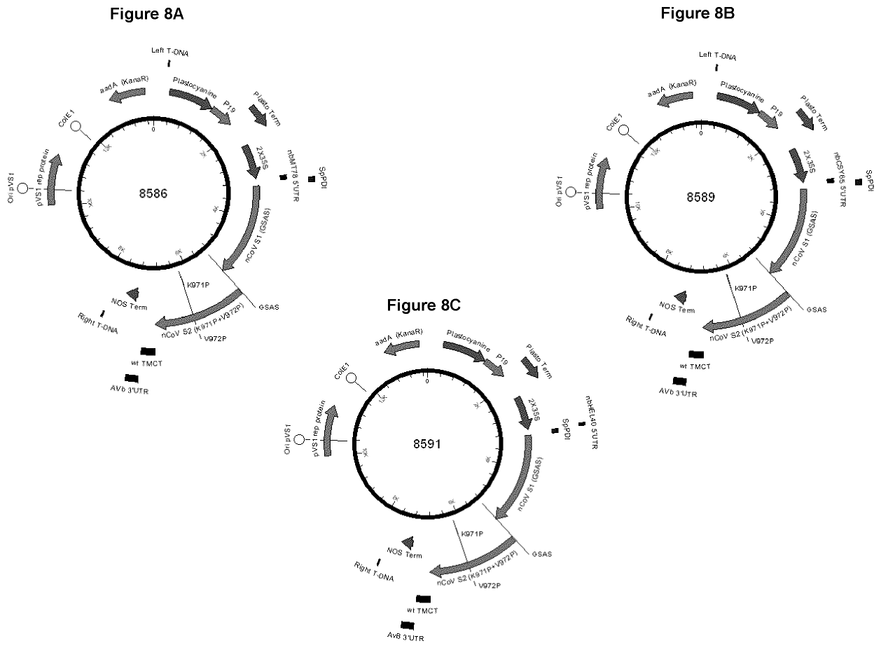

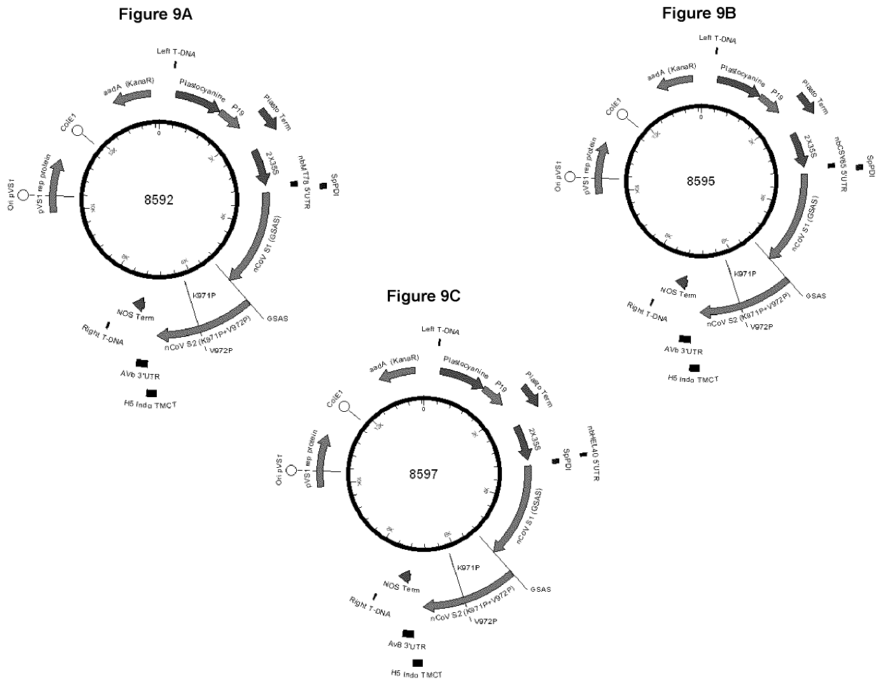

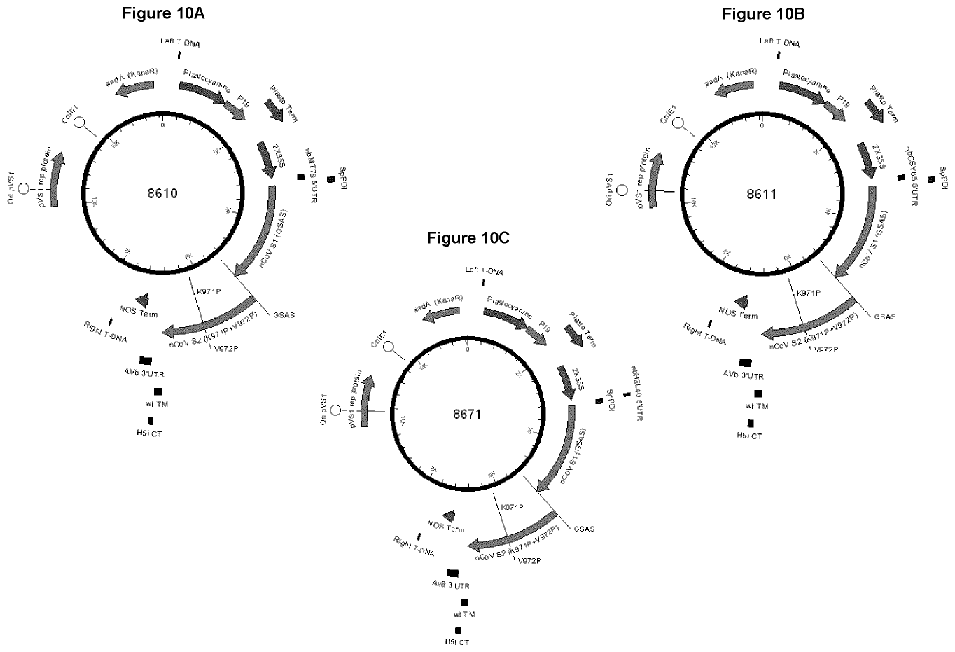

The SARS-CoV-2 S protein constructs were produced using techniques well known within the art. For example SARS-COV-2 Spike Protein with wtTMCT (Constructs number 8586, 8589, 8591, see FIG. 8A-8C) were cloned as described below. Constructs for SARS-COV-2 Spike Protein with H5iTMCT (Constructs number 8592, 8595, 8597, see FIG. 9A-9C) and constructs for SARS-COV-2 Spike Protein with H5iCT (Constructs number 8610, 8611, 8671, see FIG. 10A-10C) were obtained using similar techniques and sequences primers, templates and products are described in Table 5.

SARS-COV-2 Spike Protein with wtTMCT (Constructs Number 8586, 8589, 8591)

A sequence encoding mature SARS-CoV-2 Spike (S) protein 2 (SEQ ID NO: 23) with GSAS+K971P+V972P ectodomain mutations and with native transmembrane domain and native cytoplasmic tail (wtTMCT) from SARS-CoV-2, fused to alfalfa PDI secretion signal peptide (PDISP) was cloned into three different expression systems using the following PCR-based method. A fragment containing the SARS-COV-2 Spike protein (wtTMCT) coding sequence was amplified using primers IF(PDI)-CoV(opt2).c (SEQ ID NO: 24) and IF(AVB)-CoV(opt2).r (SEQ ID NO: 25), using PDISP-SARS-COV-2 Spike Protein with wtTMCT gene sequence (SEQ ID NO: 22) as template. The PCR product was cloned into three different expression systems using In-Fusion cloning system (Clontech, Mountain View, CA).

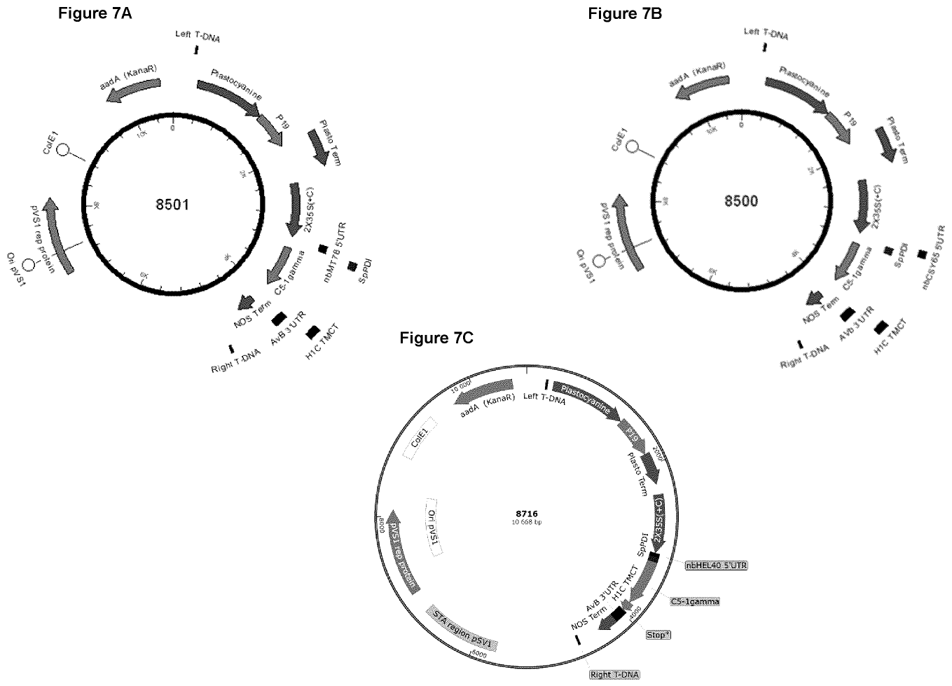

For the first expression system, construct number 8501 (FIG. 7A), was digested with AatII and StuI restriction enzymes and the linearized plasmid was used for the first In-Fusion assembly reaction. Construct number 8501 is an acceptor plasmid intended for “In Fusion” cloning of genes of interest in a 2λ35S(+C)/nbMT78/PDI/AvB/NOS-based expression cassette. This acceptor plasmid also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in SEQ ID NO: 31. The resulting construct was given number 8586 (SEQ ID NO: 32). The amino acid sequence of mature spike protein from SARS-COV-2 fused to alfalfa PDI secretion signal peptide (PDISP) is presented in SEQ ID NO: 23. A representation of plasmid 8586 is presented in FIG. 8A.

For the second expression system, construct number 8500 (FIG. 7B), was also digested with AatII and StuI restriction enzymes and the linearized plasmid was used for the second In-Fusion assembly reaction. Construct number 8500 is an acceptor plasmid intended for “In Fusion” cloning of genes of interest in a 2λ35S(+C)/nbCSY65/PDI/AvB/NOS-based expression cassette. This acceptor plasmid also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in SEQ ID NO: 33. The resulting construct was given number 8589 (SEQ ID NO: 34). The amino acid sequence of mature spike protein from SARS-COV-2 fused to alfalfa PDI secretion signal peptide (PDISP) is presented in SEQ ID NO: 23. A representation of plasmid 8589 is presented in FIG. 8B.

For the third expression system, construct number 8716 (FIG. 7C), was also digested with AatII and StuI restriction enzymes and the linearized plasmid was used for the third In-Fusion assembly reaction. Construct number 8716 is an acceptor plasmid intended for “In Fusion” cloning of genes of interest in a 2λ35S(+C)/nbHEL40/PDI/AvB/NOS based expression cassette. This acceptor plasmid also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in SEQ ID NO: 35. The resulting construct was given number 8591 (SEQ ID NO: 36). The amino acid sequence of mature spike protein from SARS-COV-2 fused to alfalfa PDI secretion signal peptide (PDISP) is presented in SEQ ID NO: 23. A representation of plasmid 8591 is presented in FIG. 8C.

SARS-COV-2 Spike Protein with H5iTMCT (Constructs Number 8592, 8595, 8597)

A sequence encoding mature Spike (S) protein from SARS-CoV-2 (SEQ ID NO: 27) with GSAS+K971P+V972P ectodomain mutations, and with transmembrane domain and cytoplasmic tail from H5 A/Indonesia/5/05 HA (H5iTMCT), was fused to alfalfa PDI secretion signal peptide (PDISP) and cloned into the same three expression systems described above by a similar PCR-based method (see table 5 for primers and Example 3 for sequences used). Construct number 8592 (FIG. 9A) was derived from acceptor construct 8501, construct number 8595 (FIG. 9B) was derived from acceptor construct 8500 and construct number 8597 (FIG. 9C) was derived from acceptor construct 8716 using similar techniques as described above and the primers, templates and products is provided in Table 5 below.

SARS-COV-2 Spike Protein with H5iCT (Constructs Number 8610, 8611, 8671)

A sequence encoding mature Spike (S) protein from SARS-CoV-2 (SEQ ID NO: 30) with GSAS+K971P+V972P ectodomain mutations, and cytoplasmic tail from H5 A/Indonesia/5/05 HA (H5iCT), was fused to alfalfa PDI secretion signal peptide (PDISP) and cloned into the same three expression systems as described above by a similar PCR-based method (see table 5 for primers and Example 3 for sequences used). Construct number 8610 (FIG. 10A) was derived from acceptor construct 8501, construct number 8611 (FIG. 10B) was derived from acceptor construct 8500 and construct number 8671 (FIG. 10C) was derived from acceptor construct 8716 using similar techniques as described above and the primers, templates and products is provided in Table 5 below.

SARS-COV-2 Spike Protein with Alternative TM CT Fusion Sequences (Constructs Number 8980, 8981, 8982, 8983)

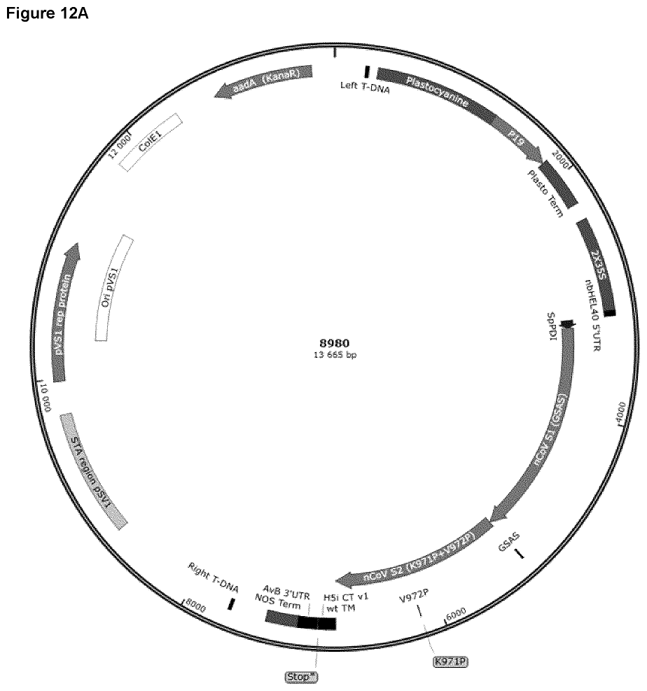

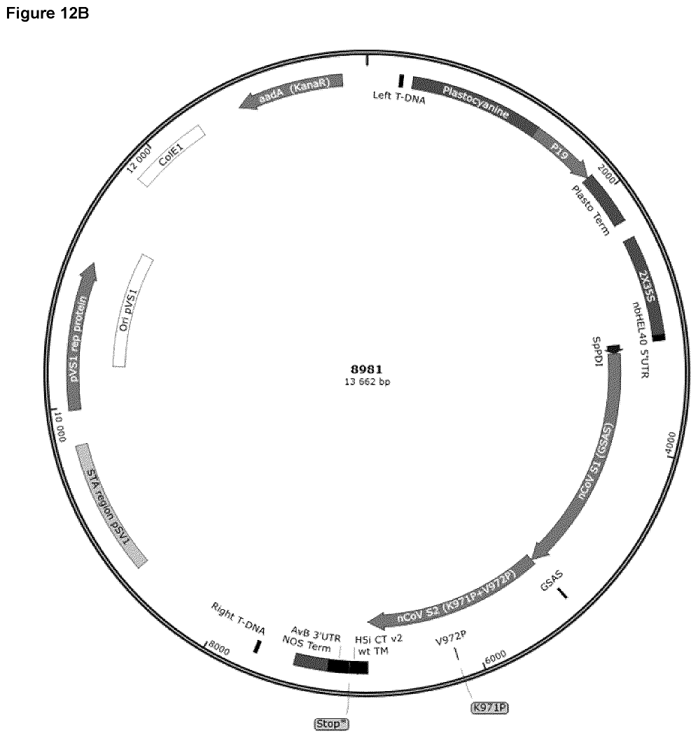

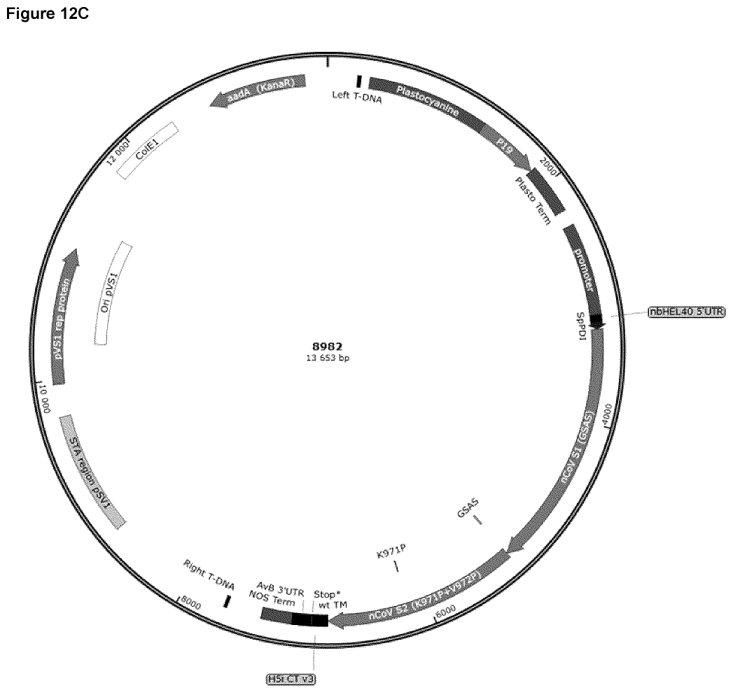

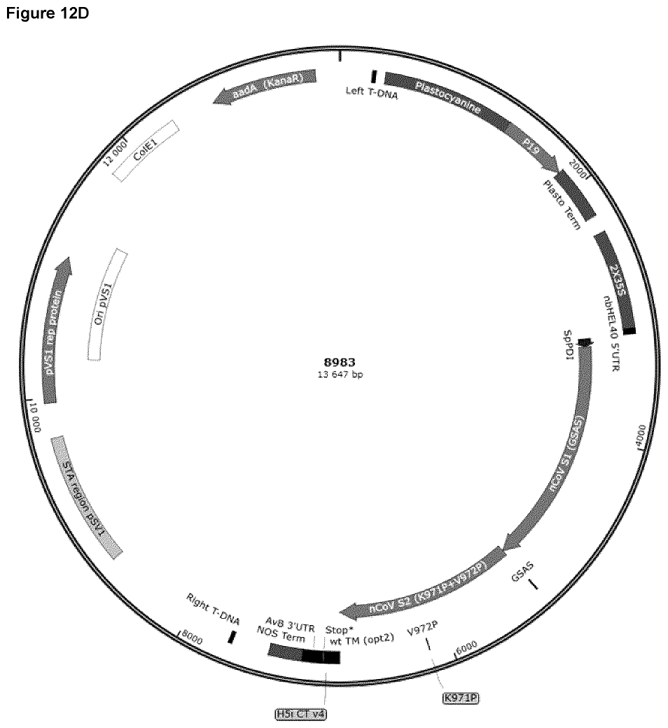

A sequence encoding mature Spike (S) protein from SARS-CoV-2 with GSAS+K971P+V972P ectodomain mutations, and cytoplasmic tail from H5 A/Indonesia/5/05 HA (H5iCT), as depicted in SEQ ID NO: 19, was fused to alfalfa PDI secretion signal peptide (PDISP) and cloned into the same expression system as described for construct 8671, yielding construct 8980 (FIG. 12A). Similar constructs were created for a sequence encoding mature Spike (S) protein from SARS-CoV-2 with GSAS+K971P+V972P ectodomain mutations, and cytoplasmic tail from H5 A/Indonesia/5/05 HA (H5iCT), as depicted in SEQ ID NO: 37 (construct 8981, FIG. 12B), SEQ ID NO: 38 (construct 8982, FIG. 12C), and SEQ ID NO: 39 (construct 8983, FIG. 12D).

SARS-COV-2 Spike Protein with CT from Other HA Strains (Constructs Number 7390, 7391, 7392, 7393, 7394, and 7395)

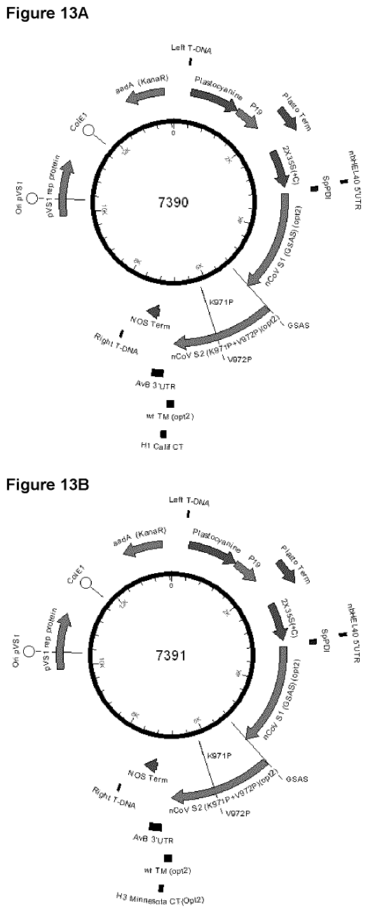

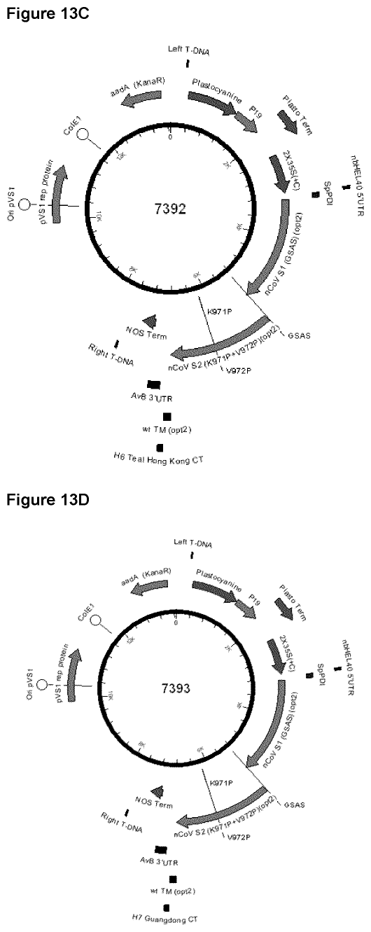

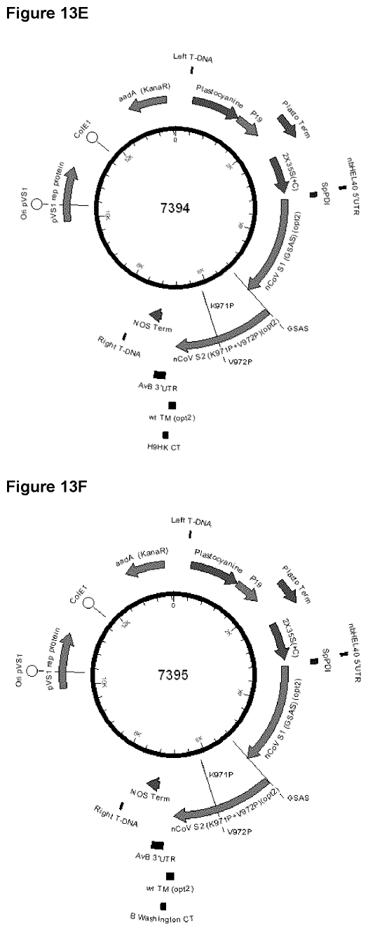

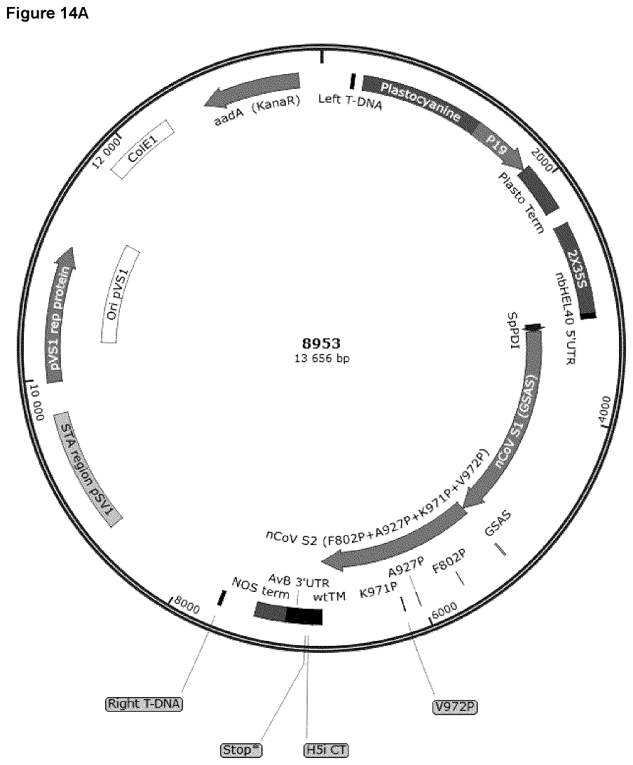

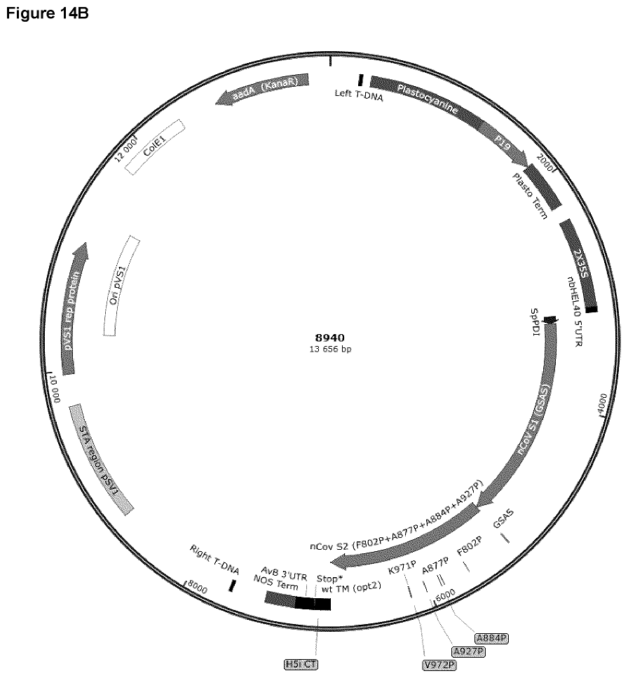

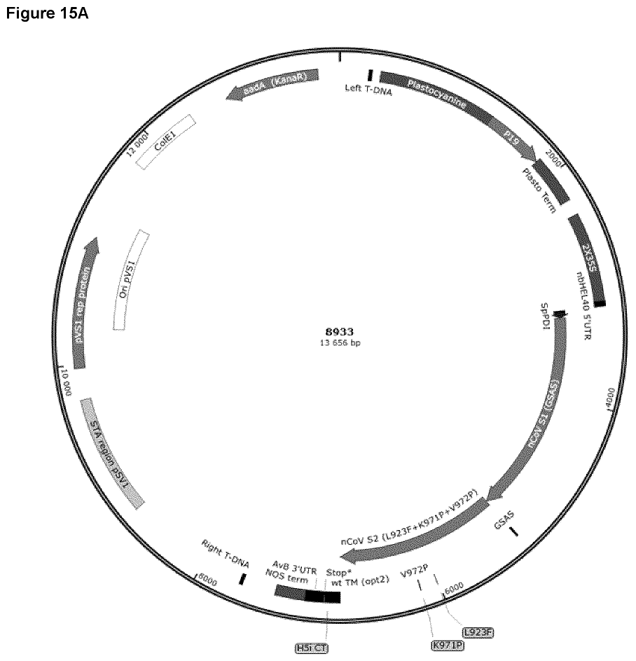

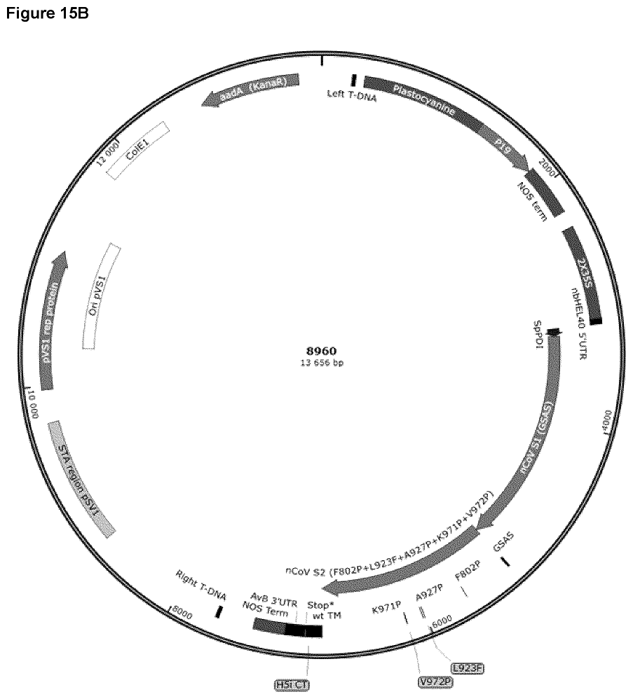

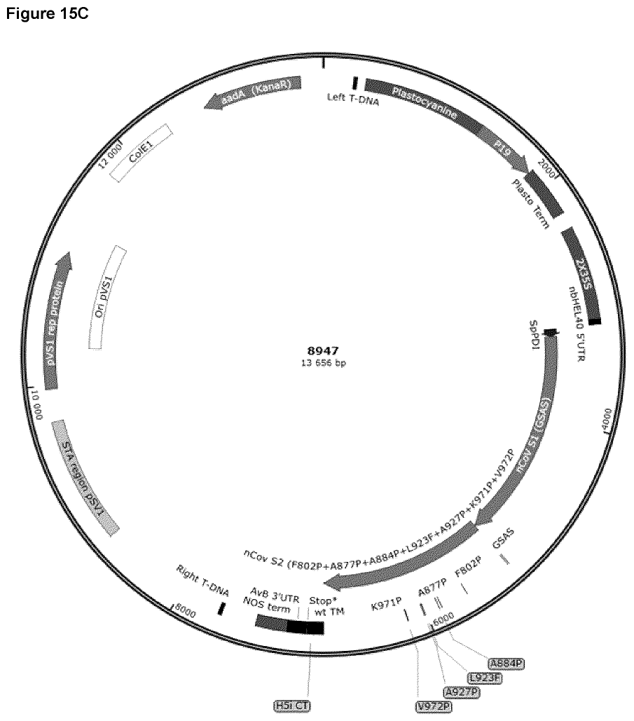

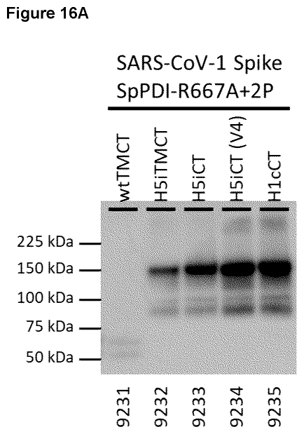

A sequence encoding mature Spike (S) protein from SARS-CoV-2 with GSAS+K971P+V972P ectodomain mutations, and cytoplasmic tail from H1 A/California/7/2009 HA (H1CT), was fused to alfalfa PDI secretion signal peptide (PDISP) and cloned into the same expression system as described for construct 8671 above by a similar PCR-based method (see table 5 for primers and Example 3 for sequences used). The resulting construct 7390 thus encodes a modified S protein comprising a H1 A/California/7/2009 HA cytoplasmic tail (H1CT) (FIG. 13A). Similar constructs were created for H3 A/Minnesota/41/2019 (Construct 7391, H3 CT) (FIG. 13B), H6 A/Teal/Hong Kong/W312/97 (Construct 7392, H6 CT) (FIG. 13C), H7 A/Guangdong/17SF003/2016 (Construct 7393, H7 CT) (FIG. 13D), H9 A/Hong Kong/1073/99 (Construct 7394, H9h CT) (FIG. 13E) or B/Washington/02/2019 (Construct 7395, HA B CT) (FIG. 13F).

SARS-COV-2 Spike Protein with Substitutions (Constructs Numbers 8933, 8960, 8947)

Modified SARS-CoV-2 S protein constructs comprising combinations of mutations in the S protein, such as R667G, R668S, R670S, F802P, A877P, A884P, A927P, K971P, V972P, and L923F were produced using techniques well known within the art and basically as described above. The constructs have the following substitutions: Construct 8933: R667G, R668S, R670S, K971P, V972P and L923F (“GSAS-2P-923”); construct 8960: R667G, R668S, R670S, F802P, A927P, K971P, V972P and L923F (“GSAS-4P-923”) and construct 8947: R667G, R668S, R670S, F802P, A877P, A884P, A927P, K971P, V972P and L923F (“GSAS-6P-923”).

SARS-COV-1 Spike Protein with wtTMCT and Modified TMCT (Constructs Number 9231, 9232, 9233, 9234, 9235)

A sequence encoding mature SARS-CoV-1 Spike (S) protein (SEQ ID NO: 88) with R654A+K955P+V956P ectodomain mutations and with native transmembrane domain and native cytoplasmic tail (wtTMCT) from SARS-CoV-1, fused to alfalfa PDI secretion signal peptide (PDISP) was cloned into the following expression system by a PCR-based method. A fragment containing the PDISP-SARS-COV-1 Spike protein (wtTMCT) coding sequence was amplified using primers IF(nbHEL40)-PDI.c (SEQ ID NO: 86) and IF(AvB+wtCT).r (SEQ ID NO: 87), using PDISP-SARS-COV-1 Spike Protein with wtTMCT gene sequence (SEQ ID NO: 88) as template. The PCR product was cloned into the following expression system using In-Fusion cloning system (Clontech, Mountain View, CA).

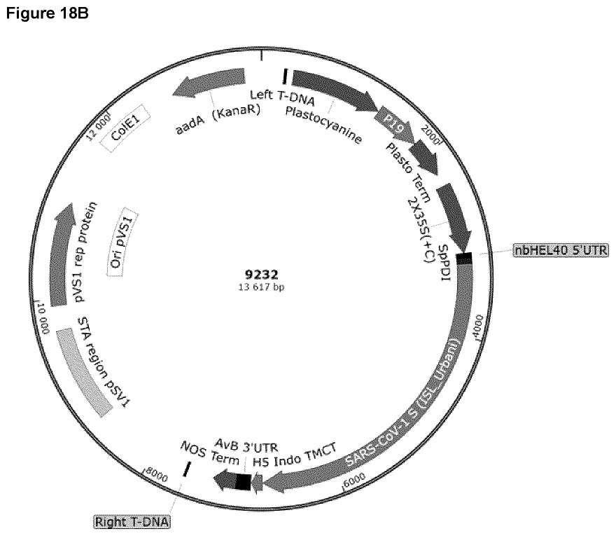

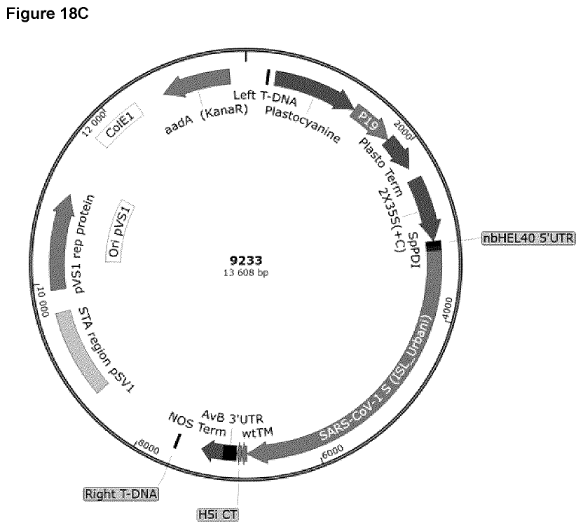

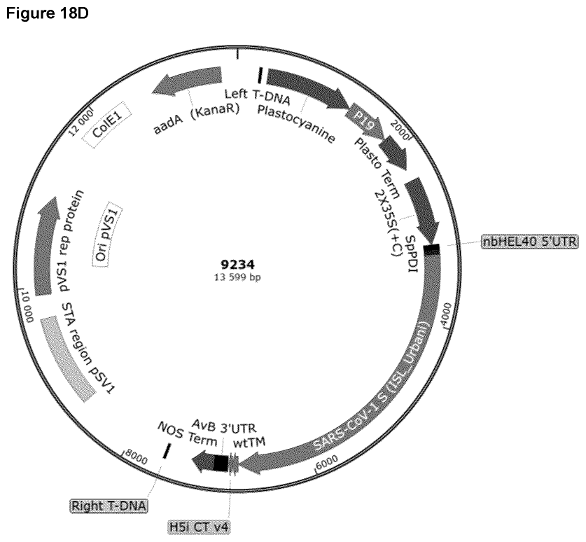

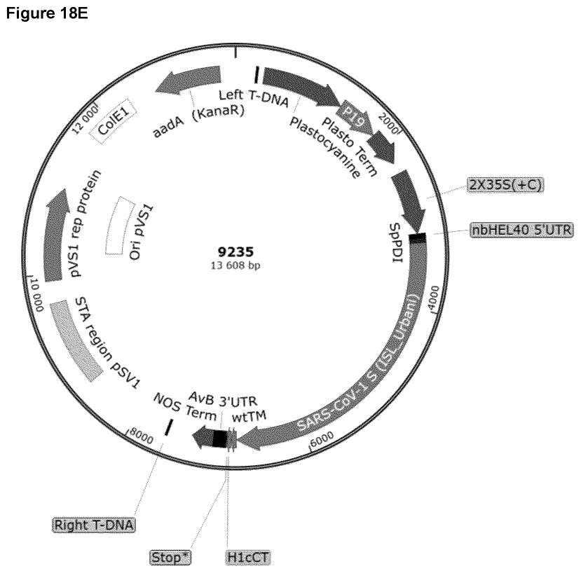

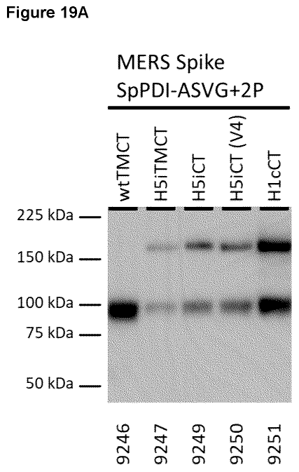

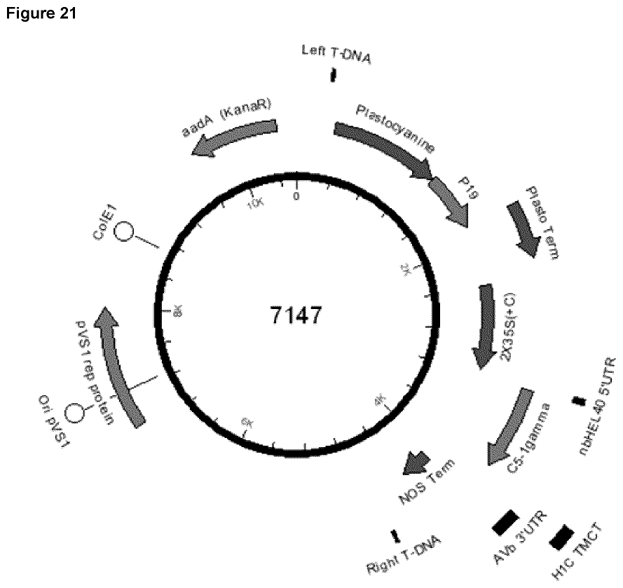

Construct number 7147 (FIG. 21) was digested with AatII and StuI restriction enzymes and the linearized plasmid was used for the first In-Fusion assembly reaction. Construct number 7147 is an acceptor plasmid intended for “In Fusion” cloning of genes of interest in a 2λ35S(+C)/nbHEL40/AvB/NOS-based expression cassette. This acceptor plasmid also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in SEQ ID NO: 111. The resulting construct was given number 9231. The amino acid sequence of mature spike protein from SARS-COV-1 fused to alfalfa PDI secretion signal peptide (PDISP) is presented in SEQ ID NO: 93. A representation of plasmid 9231 is presented in FIG. 18A.

A sequence encoding mature Spike (S) protein from SARS-CoV-1 with R654A+K955P+V956P ectodomain mutations, and either i) transmembrane domain and cytoplasmic tail from H5 A/Indonesia/5/05 HA (H5iTMCT), ii) cytoplasmic tail from H5 A/Indonesia/5/05 HA (H5iCT and variation H5iCT(V4)), or iii) cytoplasmic tail from H1 A/California/7/2009 HA (H1cCT), were fused to alfalfa PDI secretion signal peptide (PDISP) and cloned into the same expression system as described for construct 9231 above by a similar PCR-based method (see table 5 for primers and Example 3 for sequences used). The resulting constructs 9232, 9233, 9234, 9235 thus encode a modified S protein comprising a H5 A/Indonesia/5/05 TMCT (H5iTMCT) (FIG. 18B, SEQ ID NO: 94), a modified SARS-COV-1 S protein comprising a H5 A/Indonesia/5/05 CT (H5iCT) (FIG. 18C, SEQ ID NO: 95), a modified S protein comprising a H5 A/Indonesia/5/05 CT variant (H5iCT(V4)) (FIG. 18D, SEQ ID NO: 96), or a modified S protein comprising a H1 A/California/7/2009 CT (H1cCT) (FIG. 18E, SEQ ID NO: 97.)

MERS-CoV Spike Protein with wtTMCT and Modified TMCT (Constructs Number 9246, 9247, 9249, 9250, 9251)

A sequence encoding mature MERS-CoV Spike (S) protein (SEQ ID NO: 101) with R730A+R733G+V1043P+L1044P ectodomain mutations and with native transmembrane domain and native cytoplasmic tail (wtTMCT) from MERS-CoV, fused to alfalfa PDI secretion signal peptide (PDISP) was cloned into the following expression system by a PCR-based method. A fragment containing the PDISP-MERS-COV Spike protein (wtTMCT) coding sequence was amplified using primers IF(nbHEL40)-PDI.c (SEQ ID NO: 86) and IF(AvB+wtCT-MERS).r (SEQ ID NO: 98), using PDISP-MERS-COV Spike Protein with wtTMCT gene sequence (SEQ ID NO: 101) as template. The PCR product was cloned into the following expression system using In-Fusion cloning system (Clontech, Mountain View, CA).

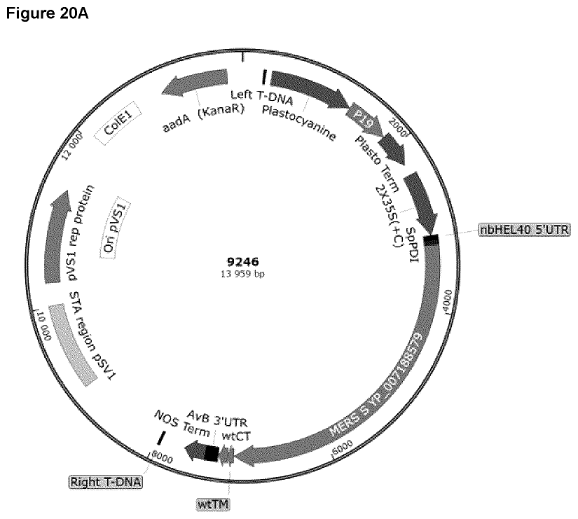

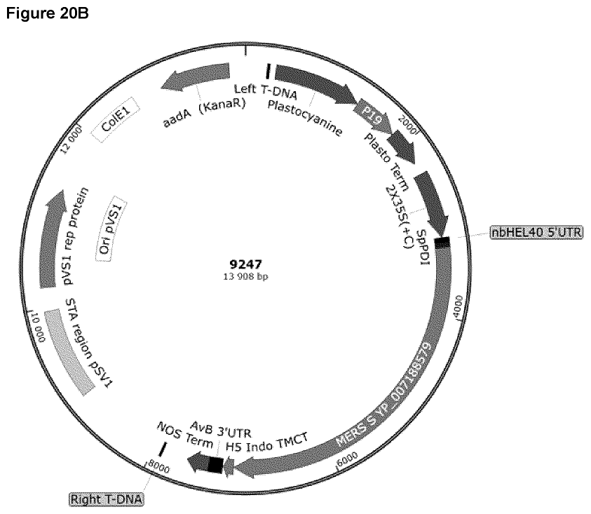

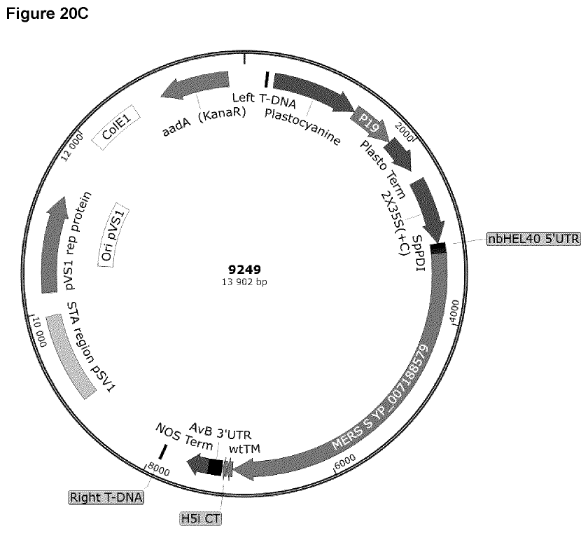

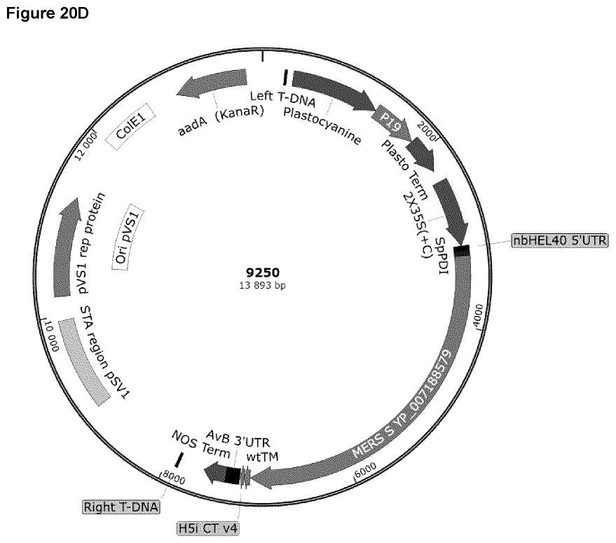

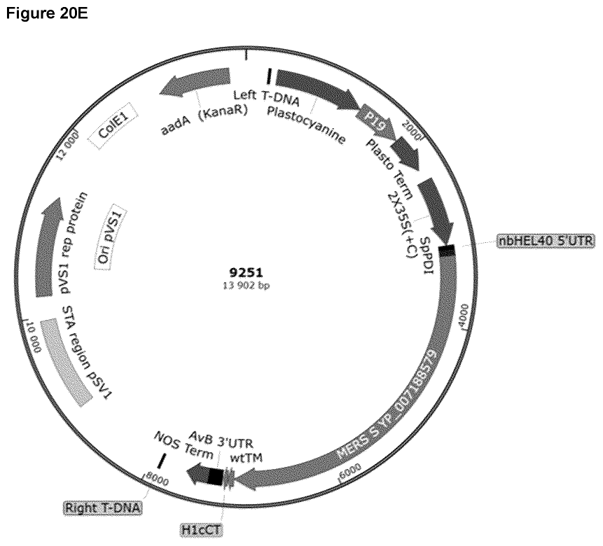

Construct number 7147 (FIG. 21) was digested with AatII and StuI restriction enzymes and the linearized plasmid was used for the first In-Fusion assembly reaction. Construct number 7147 is an acceptor plasmid intended for “In Fusion” cloning of genes of interest in a 2λ35S(+C)/nbHEL40/AvB/NOS-based expression cassette. This acceptor plasmid also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in SEQ ID NO: 111. The resulting construct was given number 9246. The amino acid sequence of mature spike protein from MERS-COV fused to alfalfa PDI secretion signal peptide (PDISP) is presented in SEQ ID NO: 106. A representation of plasmid 9246 is presented in FIG. 20A.

A sequence encoding mature Spike (S) protein from MERS-CoV with R730A+R733G+V1043P+L1044P ectodomain mutations, and either i) transmembrane domain and cytoplasmic tail from H5 A/Indonesia/5/05 HA (H5iTMCT), ii) cytoplasmic tail from H5 A/Indonesia/5/05 HA (H5iCT and variation H5iCT(V4)), or iii) cytoplasmic tail from H1 A/California/7/2009 HA (H1cCT), were fused to alfalfa PDI secretion signal peptide (PDISP) and cloned into the same expression system as described for construct 9246 above by a similar PCR-based method (see table 5 for primers and Example 3 for sequences used). The resulting constructs 9247, 9249, 9250, 9251 thus encode a modified MERS-COV S protein comprising a H5 A/Indonesia/5/05 TMCT (H5iTMCT) (FIG. 20B, SEQ ID NO: 107), a modified S protein comprising a H5 A/Indonesia/5/05 CT (H5iCT) (FIG. 20C, SEQ ID NO: 108), a modified S protein comprising a H5 A/Indonesia/5/05 CT variant (H5iCT(V4)) (FIG. 20D, SEQ ID NO: 109), or a modified S protein comprising a H1 A/California/7/2009 CT (H1cCT) (FIG. 20E, SEQ ID NO: 110.)

OC43-CoV Spike Protein with wtTMCT and Modified TMCT (Constructs Number 9269, 9270, 9272, 9273 and 9274)

A sequence encoding mature OC43-CoV Spike (S) protein (SEQ ID NO: 137) with R761G+R762G+R764G+R765S+A1077P+L1078P ectodomain mutations and with native transmembrane domain and native cytoplasmic tail (wtTMCT) from OC43-CoV, fused to alfalfa PDI secretion signal peptide (PDISP) was cloned into the following expression system by a PCR-based method. A fragment containing the PDISP-OC43-COV Spike protein (wtTMCT) coding sequence was amplified using primers IF(nbHEL40)-PDI.c (SEQ ID NO: 86) and IF(AvB+wtCT-OC43).r (SEQ ID NO: 136), using PDISP-OC43-COV Spike Protein with wtTMCT gene sequence (SEQ ID NO: 137) as template. The PCR product was cloned into the following expression system using In-Fusion cloning system (Clontech, Mountain View, CA).

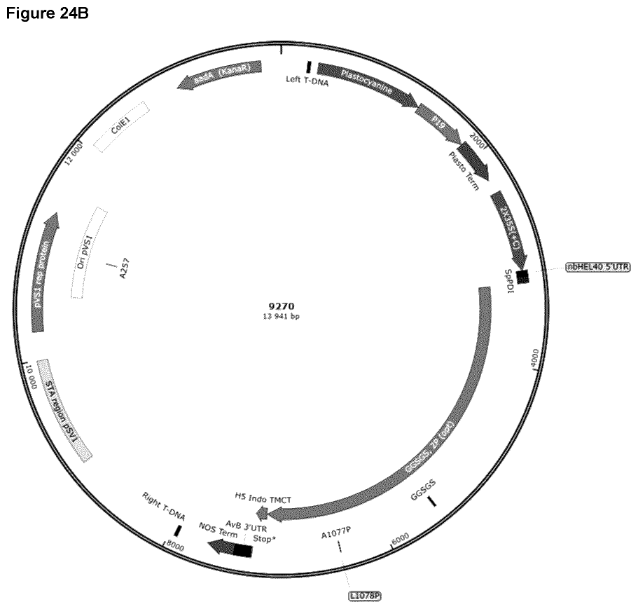

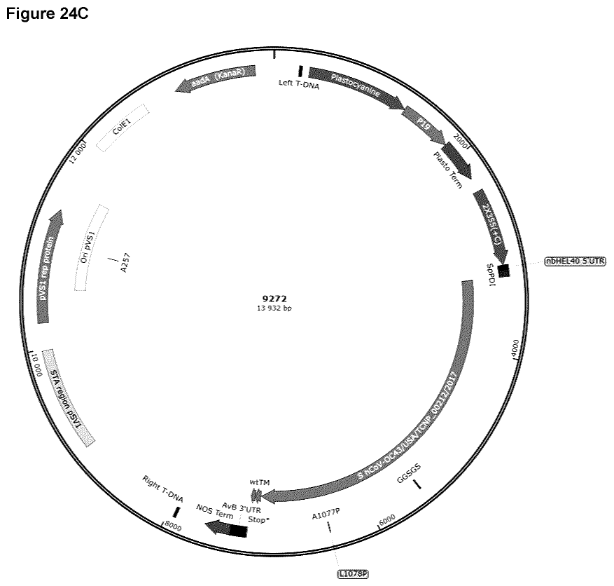

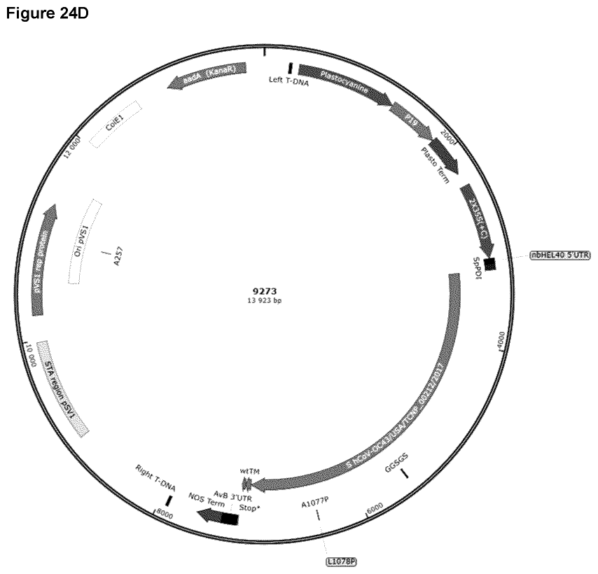

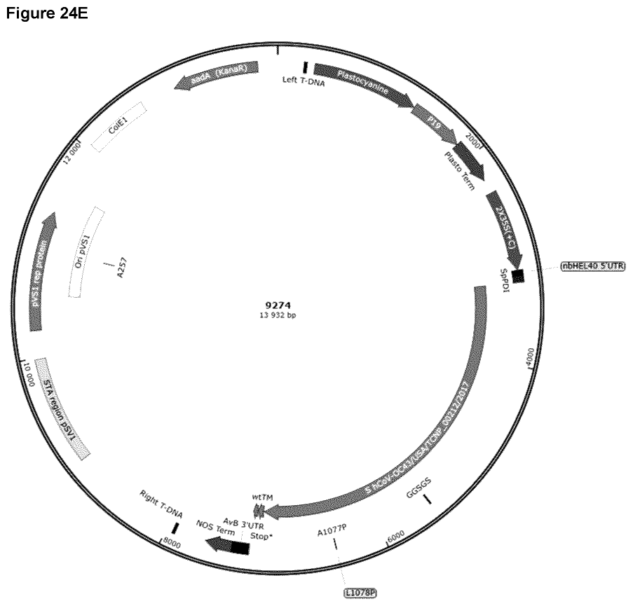

Construct number 7147 (FIG. 21) was digested with AatII and StuI restriction enzymes and the linearized plasmid was used for the first In-Fusion assembly reaction. Construct number 7147 is an acceptor plasmid intended for “In Fusion” cloning of genes of interest in a 2λ35S(+C)/nbHEL40/AvB/NOS-based expression cassette. This acceptor plasmid also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in SEQ ID NO: 111. The resulting construct was given number 9269. The amino acid sequence of mature spike protein from OC43-COV fused to alfalfa PDI secretion signal peptide (PDISP) is presented in SEQ ID NO: 142. A representation of plasmid 9269 is presented in FIG. 24A.

A sequence encoding mature Spike (S) protein from OC43-CoV with R761G+R762G+R764G+R765S+A1077P+L1078P ectodomain mutations, and either i) transmembrane domain and cytoplasmic tail from H5 A/Indonesia/5/05 HA (H5iTMCT), ii) cytoplasmic tail from H5 A/Indonesia/5/05 HA (H5iCT and variation H5iCT(V4)), or iii) cytoplasmic tail from H1 A/California/7/2009 HA (H1cCT), were fused to alfalfa PDI secretion signal peptide (PDISP) and cloned into the same expression system as described for construct 9269 above by a similar PCR-based method (see table 5 for primers and Example 3 for sequences used). The resulting constructs 9270, 9272, 9273 and 9274 thus encode a modified OC43-COV S protein comprising a H5 A/Indonesia/5/05 TMCT (H5iTMCT) (FIG. 24B, SEQ ID NO: 143), a modified S protein comprising a H5 A/Indonesia/5/05 CT (H5iCT) (FIG. 24C, SEQ ID NO: 144), a modified S protein comprising a H5 A/Indonesia/5/05 CT variant (H5iCT(V4)) (FIG. 24D, SEQ ID NO: 145), or a modified S protein comprising a H1 A/California/7/2009 CT (H1cCT) (FIG. 24E, SEQ ID NO: 146).

229E-CoV Spike Protein with wtTMCT and Modified TMCT (Constructs Number 9310, 9311, 9312, 9313 and 9314)

A sequence encoding mature 229E-CoV Spike (S) protein (SEQ ID NO: 148) with R567A+T871P+I872P ectodomain mutations and with native transmembrane domain and native cytoplasmic tail (wtTMCT) from 229E-CoV, fused to alfalfa PDI secretion signal peptide (PDISP) was cloned into the following expression system by a PCR-based method. A fragment containing the PDISP-229E-COV Spike protein (wtTMCT) coding sequence was amplified using primers IF(nbHEL40)-PDI.c (SEQ ID NO: 86) and IF(CoV229EwtCT).r (SEQ ID NO: 147), using PDISP-OC43-COV Spike Protein with wtTMCT gene sequence (SEQ ID NO: 148) as template. The PCR product was cloned into the following expression system using In-Fusion cloning system (Clontech, Mountain View, CA).

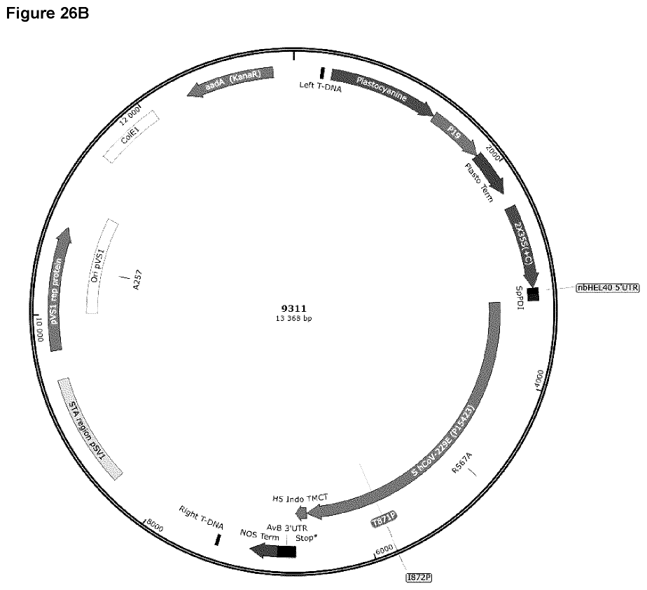

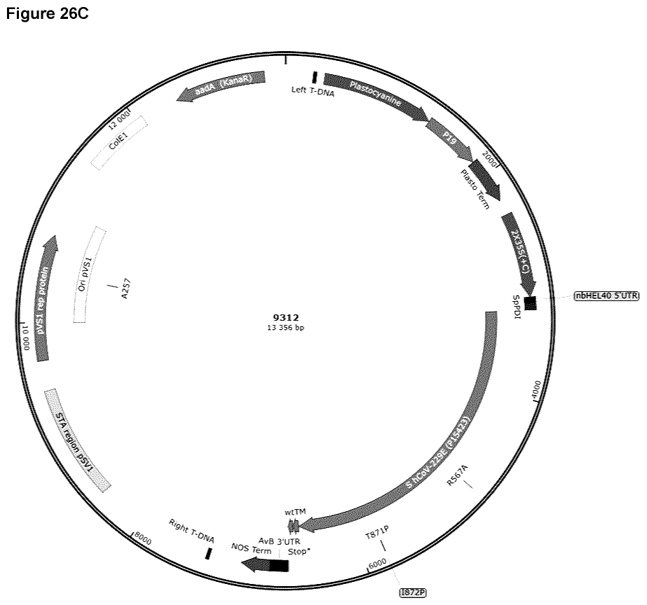

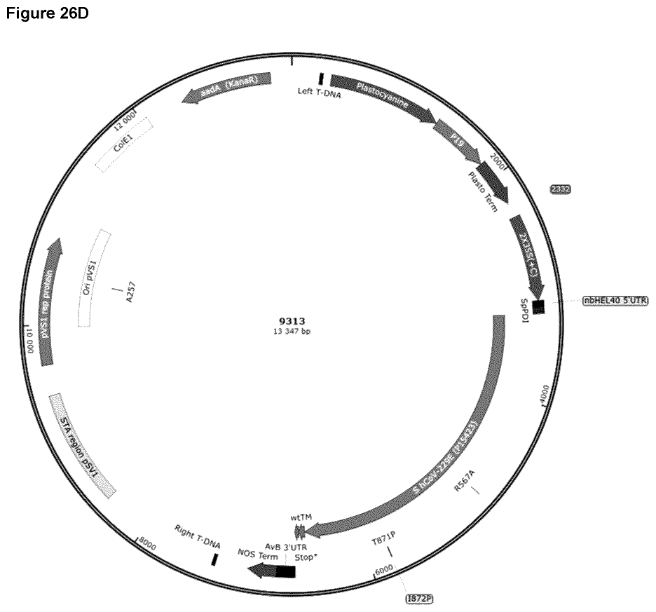

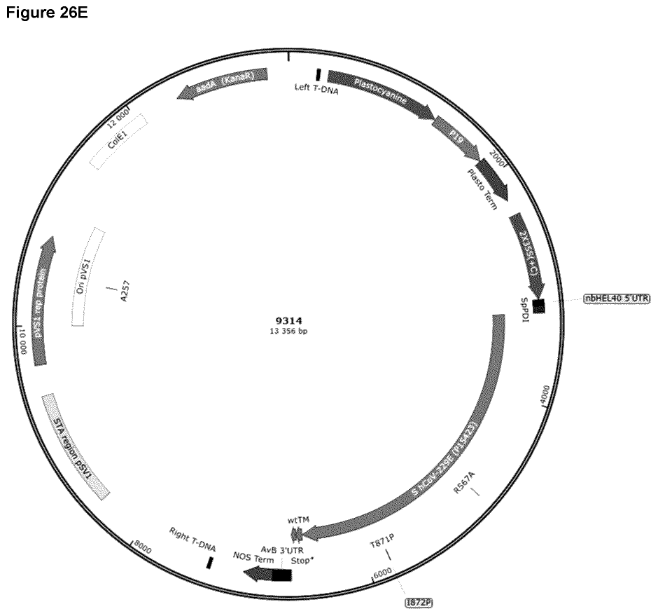

Construct number 7147 (FIG. 21) was digested with AatII and StuI restriction enzymes and the linearized plasmid was used for the first In-Fusion assembly reaction. Construct number 7147 is an acceptor plasmid intended for “In Fusion” cloning of genes of interest in a 2λ35S(+C)/nbHEL40/AvB/NOS-based expression cassette. This acceptor plasmid also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is presented in SEQ ID NO: 111. The resulting construct was given number 9310. The amino acid sequence of mature spike protein from 229E-COV fused to alfalfa PDI secretion signal peptide (PDISP) is presented in SEQ ID NO: 153. A representation of plasmid 9310 is presented in FIG. 26A.

A sequence encoding mature Spike (S) protein from 229E-CoV with R567A+T871P+I872P ectodomain mutations, and either i) transmembrane domain and cytoplasmic tail from H5 A/Indonesia/5/05 HA (H5iTMCT), ii) cytoplasmic tail from H5 A/Indonesia/5/05 HA (H5iCT and variation H5iCT(V4)), or iii) cytoplasmic tail from H1 A/California/7/2009 HA (H1cCT), were fused to alfalfa PDI secretion signal peptide (PDISP) and cloned into the same expression system as described for construct 9310 above by a similar PCR-based method (see table 5 for primers and Example 3 for sequences used). The resulting constructs 9311, 9312, 9313 and 9314 thus encode a modified 229E-COV S protein comprising a H5 A/Indonesia/5/05 TMCT (H5iTMCT) (FIG. 26B, SEQ ID NO: 154), a modified S protein comprising a H5 A/Indonesia/5/05 CT (H5iCT) (FIG. 26C, SEQ ID NO: 155), a modified S protein comprising a H5 A/Indonesia/5/05 CT variant (H5iCT(V4)) (FIG. 26D, SEQ ID NO: 156), or a modified S protein comprising a H1 A/California/7/2009 CT (H1cCT) (FIG. 26E, SEQ ID NO: 157).

TABLE 5
List of Construct Numbers, Primer and Templates

| | | | | Primer 1 | Primer 2 | Template | | |
|---|---|---|---|---|---|---|---|---|
| | | Gene of | | (forward | (reverse | for first | | |
| | | interest | | primer of | primer of | PCR/Resuting | Resulting | Acceptor |
| Construct # | 5′ UTR | (GOI) | TMCT Region | fragment 1) | fragment 1) | gene | protein | plasmid |
| 8586 | nbMT78 | SARS-CoV-2 S | wt TMCT | SEQ ID | SEQ ID | SEQ ID | SEQ ID | SEQ ID |
| | | | | NO: 24 | NO: 25 | NO: 22 | NO: 23 | NO: 31 |
| 8589 | nbCSY65 | SARS-CoV-2 S | wt TMCT | SEQ ID | SEQ ID | SEQ ID | SEQ ID | SEQ ID |
| | | | | NO: 24 | NO: 25 | NO: 22 | NO: 23 | NO: 33 |
| 8591 | nbHEL40 | SARS-CoV-2 S | wt TMCT | SEQ ID | SEQ ID | SEQ ID | SEQ ID | SEQ ID |
| | | | | NO: 24 | NO: 25 | NO: 22 | NO: 23 | NO: 35 |
| 8592 | nbMT78 | SARS-CoV-2 S | H5iTMCT | SEQ ID | SEQ ID | SEQ ID | SEQ ID | SEQ ID |
| | | | | NO: 24 | NO: 28 | NO: 26 | NO: 27 | NO: 31 |
| 8595 | nbCSY65 | SARS-CoV-2 S | H5iTMCT | SEQ ID | SEQ ID | SEQ ID | SEQ ID | SEQ ID |
| | | | | NO: 24 | NO: 28 | NO: 26 | NO: 27 | NO: 33 |
| 8597 | nbHEL40 | SARS-CoV-2 S | H5iTMCT | SEQ ID | SEQ ID | SEQ ID | SEQ ID | SEQ ID |
| | | | | NO: 24 | NO: 28 | NO: 26 | NO: 27 | NO: 35 |
| 8610 | nbMT78 | SARS-CoV-2 S | wtTM/H5iCT | SEQ ID | SEQ ID | SEQ ID | SEQ ID | SEQ ID |
| | | | | NO: 24 | NO: 28 | NO: 29 | NO: 30 | NO: 31 |
| 8611 | nbCSY65 | SARS-CoV-2 S | wtTM/H5iCT | SEQ ID | SEQ ID | SEQ ID | SEQ ID | SEQ ID |
| | | | | NO: 24 | NO: 28 | NO: 29 | NO: 30 | NO: 33 |
| 8671 | nbHEL40 | SARS-CoV-2 S | wtTM/H5iCT | SEQ ID | SEQ ID | SEQ ID | SEQ ID | SEQ ID |
| | | | | NO: 24 | NO: 28 | NO: 29 | NO: 30 | NO: 35 |
| 8953 | nbHEL40 | SARS-CoV-2 S | wtTM/H5iCT | SEQ ID | SEQ ID | SEQ ID | SEQ ID | SEQ ID |
| | | | | NO: 24 | NO: 28 | NO: 65 | NO: 48 | NO: 35 |
| 8940 | nbHEL40 | SARS-CoV-2 S | wtTM/H5iCT | SEQ ID | SEQ ID | SEQ ID | SEQ ID | SEQ ID |
| | | | | NO: 24 | NO: 28 | NO: 66 | NO: 49 | NO: 35 |
| 8933 | nbHEL40 | SARS-CoV-2 S | wtTM/H5iCT | SEQ ID | SEQ ID | SEQ ID | SEQ ID | SEQ ID |
| | | | | NO: 24 | NO: 28 | NO: 67 | NO: 50 | NO: 35 |
| 8960 | nbHEL40 | SARS-CoV-2 S | wtTM/H5iCT | SEQ ID | SEQ ID | SEQ ID | SEQ ID | SEQ ID |
| | | | | NO: 24 | NO: 28 | NO: 68 | NO: 51 | NO: 35 |
| 8947 | nbHEL40 | SARS-CoV-2 S | wtTM/H5iCT | SEQ ID | SEQ ID | SEQ ID | SEQ ID | SEQ ID |
| | | | | NO: 24 | NO: 28 | NO: 69 | NO: 52 | NO: 35 |
| 8980 | nbHEL40 | SARS-CoV-2 S | wtTM/H5iCT (V1) | SEQ ID | SEQ ID | SEQ ID | SEQ ID | SEQ ID |
| | | | | NO: 24 | NO: 28 | NO: 70 | NO: 59 | NO: 35 |
| 8981 | nbHEL40 | SARS-CoV-2 S | wtTM/H5iCT (V2) | SEQ ID | SEQ ID | SEQ ID | SEQ ID | SEQ ID |
| | | | | NO: 24 | NO: 28 | NO: 71 | NO: 60 | NO: 35 |
| 8982 | nbHEL40 | SARS-CoV-2 S | wtTM/H5iCT (V3) | SEQ ID | SEQ ID | SEQ ID | SEQ ID | SEQ ID |
| | | | | NO: 24 | NO: 28 | NO: 72 | NO: 61 | NO: 35 |
| 8983 | nbHEL40 | SARS-CoV-2 S | wtTM/H5iCT (V4) | SEQ ID | SEQ ID | SEQ ID | SEQ ID | SEQ ID |
| | | | | NO: 24 | NO: 28 | NO: 73 | NO: 62 | NO: 35 |
| 7390 | nbHEL40 | SARS-CoV-2 S | wtTM/H1cCT | SEQ ID | SEQ ID | SEQ ID | SEQ ID | SEQ ID |
| | | | | NO: 24 | NO: 80 | NO: 74 | NO: 53 | NO: 35 |
| 7391 | nbHEL40 | SARS-CoV-2 S | wtTM/H3mCT | SEQ ID | SEQ ID | SEQ ID | SEQ ID | SEQ ID |
| | | | | NO: 24 | NO: 81 | NO: 75 | NO: 54 | NO: 35 |
| 7392 | nbHEL40 | SARS-CoV-2 S | wtTM/H6hkCT | SEQ ID | SEQ ID | SEQ ID | SEQ ID | SEQ ID |
| | | | | NO: 24 | NO: 82 | NO: 76 | NO: 55 | NO: 35 |
| 7393 | nbHEL40 | SARS-CoV-2 S | wtTM/H7gCT | SEQ ID | SEQ ID | SEQ ID | SEQ ID | SEQ ID |
| | | | | NO: 24 | NO: 83 | NO: 77 | NO: 56 | NO: 35 |
| 7394 | nbHEL40 | SARS-CoV-2 S | wtTM/H9hkCT | SEQ ID | SEQ ID | SEQ ID | SEQ ID | SEQ ID |
| | | | | NO: 24 | NO: 84 | NO: 78 | NO: 57 | NO: 35 |
| 7395 | nbHEL40 | SARS-CoV-2 S | wtTM/BwCT | SEQ ID | SEQ ID | SEQ ID | SEQ ID | SEQ ID |
| | | | | NO: 24 | NO: 85 | NO: 79 | NO: 58 | NO: 35 |
| 9231 | nbHEL40 | SARS-CoV-1 S | wtTMCT | SEQ ID | SEQ ID | SEQ ID | SEQ ID | SEQ ID |
| | | | | NO: 86 | NO: 87 | NO: 88 | NO: 93 | NO: 111 |
| 9232 | nbHEL40 | SARS-CoV-1 S | H5iTMCT | SEQ ID | SEQ ID | SEQ ID | SEQ ID | SEQ ID |
| | | | | NO: 86 | NO: 28 | NO: 89 | NO: 94 | NO: 111 |
| 9233 | nbHEL40 | SARS-CoV-1 S | wtTM/H5iCT | SEQ ID | SEQ ID | SEQ ID | SEQ ID | SEQ ID |
| | | | | NO: 86 | NO: 28 | NO: 90 | NO: 95 | NO: 111 |
| 9234 | nbHEL40 | SARS-CoV-1 S | wtTM/H5iCT (V4) | SEQ ID | SEQ ID | SEQ ID | SEQ ID | SEQ ID |
| | | | | NO: 86 | NO: 28 | NO: 91 | NO: 96 | NO: 111 |
| 9235 | nbHEL40 | SARS-CoV-1 S | wtTM/H1cCT | SEQ ID | SEQ ID | SEQ ID | SEQ ID | SEQ ID |
| | | | | NO: 86 | NO: 80 | NO: 92 | NO: 97 | NO: 111 |
| 9246 | nbHEL40 | MERS S | wtTMCT | SEQ ID | SEQ ID | SEQ ID | SEQ ID | SEQ ID |
| | | | | NO: 86 | NO: 98 | NO: 101 | NO: 106 | NO: 111 |
| 9247 | nbHEL40 | MERS S | H5iTMCT | SEQ ID | SEQ ID | SEQ ID | SEQ ID | SEQ ID |
| | | | | NO: 86 | NO: 100 | NO: 102 | NO: 107 | NO: 111 |
| 9249 | nbHEL40 | MERS S | wtTM/H5iCT | SEQ ID | SEQ ID | SEQ ID | SEQ ID | SEQ ID |
| | | | | NO: 86 | NO: 100 | NO: 103 | NO: 108 | NO: 111 |
| 9250 | nbHEL40 | MERS S | wtTM/H5iCT (V4) | SEQ ID | SEQ ID | SEQ ID | SEQ ID | SEQ ID |
| | | | | NO: 86 | NO: 100 | NO: 104 | NO: 109 | NO: 111 |
| 9251 | nbHEL40 | MERS S | wtTM/H1cCT | SEQ ID | SEQ ID | SEQ ID | SEQ ID | SEQ ID |
| | | | | NO: 86 | NO: 99 | NO: 105 | NO: 110 | NO: 111 |
| 9269 | nbHEL40 | OC43 S | wtTMCT | SEQ ID | SEQ ID | SEQ ID | SEQ ID | SEQ ID |
| | | | | NO: 86 | NO: 136 | NO: 137 | NO: 142 | NO: 111 |
| 9270 | nbHEL40 | OC43 S | H5iTMCT | SEQ ID | SEQ ID | SEQ ID | SEQ ID | SEQ ID |
| | | | | NO: 86 | NO: 100 | NO: 138 | NO: 143 | NO: 111 |
| 9272 | nbHEL40 | OC43 S | wtTM/H5iCT | SEQ ID | SEQ ID | SEQ ID | SEQ ID | SEQ ID |
| | | | | NO: 86 | NO: 100 | NO: 139 | NO: 144 | NO: 111 |
| 9273 | nbHEL40 | OC43 S | wtTM/H5iCT (V4) | SEQ ID | SEQ ID | SEQ ID | SEQ ID | SEQ ID |
| | | | | NO: 86 | NO: 100 | NO: 140 | NO: 145 | NO: 111 |
| 9274 | nbHEL40 | OC43 S | wtTM/H1cCT | SEQ ID | SEQ ID | SEQ ID | SEQ ID | SEQ ID |
| | | | | NO: 86 | NO: 99 | NO: 141 | NO: 146 | NO: 111 |
| 9310 | nbHEL40 | 229E S | wtTMCT | SEQ ID | SEQ ID | SEQ ID | SEQ ID | SEQ ID |
| | | | | NO: 86 | NO: 147 | NO: 148 | NO: 153 | NO: 111 |
| 9311 | nbHEL40 | 229E S | H5iTMCT | SEQ ID | SEQ ID | SEQ ID | SEQ ID | SEQ ID |
| | | | | NO: 86 | NO: 100 | NO: 149 | NO: 154 | NO: 111 |
| 9312 | nbHEL40 | 229E S | WtTM/H5iCT | SEQ ID | SEQ ID | SEQ ID | SEQ ID | SEQ ID |
| | | | | NO: 86 | NO: 100 | NO: 150 | NO: 155 | NO: 111 |
| 9313 | nbHEL40 | 229E S | wtTM/H5iCT (V4) | SEQ ID | SEQ ID | SEQ ID | SEQ ID | SEQ ID |
| | | | | NO: 86 | NO: 100 | NO: 151 | NO: 156 | NO: 111 |
| 9314 | nbHEL40 | 229E S | wtTM/H1cCT | SEQ ID | SEQ ID | SEQ ID | SEQ ID | SEQ ID |
| | | | | NO: 86 | NO: 99 | NO: 152 | NO: 157 | NO: 111 |

### Example 2: Methods

Agrobacterium tumefaciens Transfection

Agrobacterium tumefaciens strain AGL1 was transfected by electroporation with the SARS-CoV-2 modified S protein expression vectors using the methods described by D'Aoust et al., 2008 (Plant Biotech. J 6:930-40). Transfected Agrobacterium were grown in YEB medium supplemented with 10 mM 2-(N-morpholino)ethanesulfonic acid (MES), 20 μM acetosyringone, 50 μg/ml kanamycin and 25 μg/ml of carbenicillin pH5.6 to an OD₆₀₀ between 0.6 and 1.6. Agrobacterium suspensions were centrifuged before use and resuspended in infiltration medium (10 mM MgCl₂ and 10 mM MES pH 5.6).

### Preparation of Plant Biomass, Inoculum and Agroinfiltration

N. benthamiana plants were grown from seeds in flats filled with a commercial peat moss substrate. The plants were allowed to grow in the greenhouse under a 16/8 photoperiod and a temperature regime of 25° C. day/20° C. night. Three weeks after seeding, individual plantlets were picked out, transplanted in pots and left to grow in the greenhouse for three additional weeks under the same environmental conditions.

Agrobacteria transfected with each expression vector were grown in a YEB medium supplemented with 10 mM 2-(N-morpholino)ethanesulfonic acid (MES), 20 μM acetosyringone, 50 μg/ml kanamycin and 25 μg/ml of carbenicillin pH 5.6 until they reached an OD₆₀₀ between 0.6 and 1.6. Agrobacterium suspensions were centrifuged before use and resuspended in infiltration medium (10 mM MgCl₂ and 10 mM MES pH 5.6) and stored overnight at 4° C. On the day of infiltration, culture batches were diluted in 2.5 culture volumes and allowed to warm before use. Whole plants of N. benthamiana were placed upside down in the bacterial suspension in an air-tight stainless steel tank under a vacuum of 20-40 Torr for 2-min. Plants were returned to the greenhouse for a 6 or 9 day incubation period until harvest.

### Leaf Harvest and Total Protein and VLP Extraction

Following incubation, the aerial part of plants was harvested, frozen at −80° C. and crushed into pieces. Total soluble proteins were extracted by mechanically homogenizing (Polytron) each sample of frozen-crushed plant material in two volumes of cold 50 mM Tris buffer at pH 8.0+500 mM NaCl, 0.4 μg/ml metabisulfite and 1 mM phenylmethanesulfonyl fluoride. After homogenization, the slurries were centrifuged at 10,000 g for 10 min at 4° C. and these clarified crude extracts (supernatant) kept for analysis.

The total protein content of clarified crude extracts was determined by the Bradford assay (Bio-Rad, Hercules, California) using bovine serum albumin as the reference standard. Proteins were separated by SDS-PAGE under reducing conditions using Criterion™ TGX Stain-Free™ precast gels (Bio-Rad Laboratories, Hercules, CA). Proteins were visualized by staining the gels with Coomassie Brilliant Blue. Alternatively, proteins were visualized with Gel Doc™ EZ imaging system (Bio-Rad Laboratories, Hercules, CA) and electrotransferred onto polyvinylene difluoride (PVDF) membranes (Roche Diagnostics Corporation, Indianapolis, Indiana) for immunodetection. Prior to immunoblotting, the membranes were blocked with 5% skim milk and 0.1% Tween-20 in Tris-buffered saline (TBS-T) for 16-18 h at 4° C.

For VLP purification, proteins were extracted from frozen biomass by mechanical extraction using a blender with two volumes of extraction buffer (50 mM Tris buffer at pH 7.0+500 mM NaCl) and pH was lowered to 6.1 using 0.5M citric acid. The slurry was filtered through a large pore nylon filter to remove large debris and centrifuged 5000 g for 5 min at 4° C. The supernatant was collected and centrifuged again at 5000 g for 30 min (4° C.) to remove additional debris and passed through clarification filters. The supernatant was then loaded on a discontinuous iodixanol density gradient. Analytical density gradient centrifugation was performed as follows: 38 mL tubes containing discontinuous iodixanol density gradient in Tris buffer (3 ml at 35%, 3 ml at 30%, 3 ml at 25%, 3 ml at 15% and 5 ml at 10% of iodixanol) were prepared and overlaid with 22 ml of the extracts containing the virus-like particles. The gradients were centrifuged at 120 000 g for 2 hours (4° C.). After centrifugation, 1 mL fractions were collected from the bottom to the top and fractions were analyzed by SDS-PAGE combined with protein staining or Western blot. Fractions 6 to 9 were pooled and buffer-exchanged using Amicon centrifugation device. Protein content is determined by Bradford assay.

### Protein Analysis and Immunoblotting

Immunoblotting was performed with a first incubation with a primary mAb, (anti-S1, Sino Biological, cat #40150-R007 or anti-S2, Novus biological, cat #NB100-56578) diluted in 2% skim milk in TBS-Tween 20 0.1%. Peroxydase-conjugated goat anti-rabbit (Jackson Immunoresearch, cat #115-035-144) was used as secondary antibody for chemiluminescence detection in 2% skim milk in TBS-Tween 20 0.1% Immunoreactive complexes were detected by chemiluminescence using luminol as the substrate (Roche Diagnostics Corporation). Horseradish peroxidase-enzyme conjugation of human IgG antibody was carried out by using the EZ-Link Plus® Activated Peroxidase conjugation kit (Pierce, Rockford, Ill.).

In planta yields were assessed on clarified crude extracts and analyzed using a capillary-based electrophoresis method (Protein Simple, BioTechne) technology and a WES analysis system. In brief, soluble proteins from crude extracts were separated by molecular weight in a capillary and fixed to the matrix. A standard curve using purified VLPs is used to determine S protein quantity and Anti-S2 antibody (Novus biological, cat #NB100-56578) is used for detection according to the manufacturer instructions. Yields are then normalized using a comparator construct which is set to 1.

The primary antibody for detection of SARS-CoV S protein was SARS-CoV Spike S1 subunit antibody from Sino Biologicals, 40150-MM08 (1/5000) and the secondary antibody used for detection was Goat anti-Mouse, JIR, 115-035-146 (1/10000). The primary antibody for detection of MERS CoV S protein was MERS-CoV spike protein S1 antibody (N-terminal) from Sino Biological, (100208-RP02, 1/5000). The secondary antibody used for detection was Goat anti-Mouse from JIR (115-035-144, 1/10000). The primary antibody used for detection was anti-coronavirus OC43 spike protein from Antibodies-online (ABIN2754654, 1/1000. The secondary antibody used for detection was Goat anti-Rabbit from JIR (111-035-144, 1/10000).

### Electron Microscopy

To determine whether expressed S protein assembled into VLPs, transmission electron microscopy (TEM) of immuno-trapped particles was performed on purified VLPs. Glow discharged carbon/copper grids (10 s, 0.3 mbar) were placed on 20 μL of purified VLPs (100 μg/mL) for 5 min and then washed 4 times with sterile distilled water. The grids were floated on 20 μL of 2% uranyl acetate for 1 min, excess solution is then removed by touching a moist filter paper and allowed to dry for 24 h on a filter paper before viewing under a TEM (Tecnai Microscope).

### Example 3: Sequences

The following sequences were use in the examples described above.

Native SARS-CoV-2 S protein wtTM/CT AA (P0DTC2)
(SEQ ID NO: 1)
MFVFLVLLPLVSSQCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSY
LTPGDSSSGWTAGAAAYYVGYLQPRTFLLKYNENGTITDAVDCALDPLSETKCTLKSFTVEK
GIYQTSNFRVQPTESIVRFPNITNLCPFGEVFNATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGFNCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPR
RARSVASQSIIAYTMSLGAENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLTDE
MIAQYTSALLAGTITSGWTFGAGAALQIPFAMQMAYRFNGIGVTQNVLYENQKLIANQFNSA
IGKIQDSLSSTASALGKLQDVVNQNAQALNTLVKQLSSNFGAISSVLNDILSRLDKVEAEVQ
IDRLITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINASVVN
IQKEIDRLNEVAKNLNESLIDLQELGKYEQYIKWPWYIWLGFIAGLIAIVMVTIMLCCMTSC
CSCLKGCCSCGSCCKFDEDDSEPVLKGVKLHYT
Native SARS-CoV-2 S protein wtTM/CT AA (P0DTC2) without signal peptide (SP)
(SEQ ID NO: 2)
VNLTTRTQLPPAYTNSFTRGVYYPDKVFRSSVLHSTQDLFLPFFSNVTWFHAIHVSGTNGTK
RFDNPVLPFNDGVYFASTEKSNIIRGWIFGTTLDSKTQSLLIVNNATNVVIKVCEFQFCNDP
FLGVYYHKNNKSWMESEFRVYSSANNCTFEYVSQPFLMDLEGKQGNFKNLREFVFKNIDGYF
KIYSKHTPINLVRDLPQGFSALEPLVDLPIGINITRFQTLLALHRSYLTPGDSSSGWTAGAA
AYYVGYLQPRTFLLKYNENGTITDAVDCALDPLSETKCTLKSFTVEKGIYQTSNFRVQPTES
IVRFPNITNLCPFGEVFNATRFASVYAWNRKRISNCVADYSVLYNSASFSTFKCYGVSPTKL
NDLCFTNVYADSFVIRGDEVRQIAPGQTGKIADYNYKLPDDFTGCVIAWNSNNLDSKVGGNY
NYLYRLFRKSNLKPFERDISTEIYQAGSTPCNGVEGFNCYFPLQSYGFQPTNGVGYQPYRVV
VLSFELLHAPATVCGPKKSTNLVKNKCVNFNFNGLTGTGVLTESNKKFLPFQQFGRDIADTT
DAVRDPQTLEILDITPCSFGGVSVITPGTNTSNQVAVLYQDVNCTEVPVAIHADQLTPTWRV
YSTGSNVFQTRAGCLIGAEHVNNSYECDIPIGAGICASYQTQTNSPRRARSVASQSIIAYTM
SLGAENSVAYSNNSIAIPTNFTISVTTEILPVSMTKTSVDCTMYICGDSTECSNLLLQYGSF
CTQLNRALTGIAVEQDKNTQEVFAQVKQIYKTPPIKDFGGFNFSQILPDPSKPSKRSFIEDL
LFNKVTLADAGFIKQYGDCLGDIAARDLICAQKFNGLTVLPPLLTDEMIAQYTSALLAGTIT
SGWTFGAGAALQIPFAMQMAYRFNGIGVTQNVLYENQKLIANQFNSAIGKIQDSLSSTASAL
GKLQDVVNQNAQALNTLVKQLSSNFGAISSVLNDILSRLDKVEAEVQIDRLITGRLQSLQTY
VTQQLIRAAEIRASANLAATKMSECVLGQSKRVDFCGKGYHLMSFPQSAPHGVVFLHVTYVP
AQEKNFTTAPAICHDGKAHFPREGVFVSNGTHWFVTQRNFYEPQIITTDNTFVSGNCDVVIG
IVNNTVYDPLQPELDSFKEELDKYFKNHTSPDVDLGDISGINASVVNIQKEIDRLNEVAKNL
NESLIDLQELGKYEQYIKWPWYIWLGFIAGLIAIVMVTIMLCCMTSCCSCLKGCCSCGSCCK
FDEDDSEPVLKGVKLHYT
H5 A/Indonesia/5/05 Hemagglutinin (HA) AA (A5A5L7)
(SEQ ID NO: 3)
MEKIVLLLAIVSLVKSDQICIGYHANNSTEQVDTIMEKNVTVTHAQDILEKTHNGKLCDLDG
VKPLILRDCSVAGWLLGNPMCDEFINVPEWSYIVEKANPTNDLCYPGSFNDYEELKHLLSRI
NHFEKIQIIPKSSWSDHEASSGVSSACPYLGSPSFFRNVVWLIKKNSTYPTIKKSYNNTNQE
DLLVLWGIHHPNDAAEQTRLYONPTTYISIGTSTLNQRLVPKIATRSKVNGQSGRMEFFWTI
LKPNDAINFESNGNFIAPEYAYKIVKKGDSAIMKSELEYGNCNTKCQTPMGAINSSMPFHNI
HPLTIGECPKYVKSNRLVLATGLRNSPQRESRRKKRGLFGAIAGFIEGGWQGMVDGWYGYHH
SNEQGSGYAADKESTQKAIDGVTNKVNSIIDKMNTQFEAVGREFNNLERRIENLNKKMEDGF
LDVWTYNAELLVLMENERTLDFHDSNVKNLYDKVRLQLRDNAKELGNGCFEFYHKCDNECME
SIRNGTYNYPQYSEEARLKREEISGVKLESIGTYQILSIYSTVASSLALAIMMAGLSLWMCS
NGSLQCRICI
H5 A/Indonesia/5/05 Hemagglutinin (HA) viral cDNA (EF541394.1)
(SEQ ID NO: 4)
CTGTAAAAATGGAGAAAATAGTGCTTCTTCTTGCAATAGTCAGTCTTGTTAAAAGTGATCAG
ATTTGCATTGGTTACCATGCAAACAATTCAACAGAGCAGGTTGACACAATCATGGAAAAGAA
CGTTACTGTTACACATGCCCAAGACATACTGGAAAAGACACACAACGGGAAGCTCTGCGATC
TAGATGGAGTGAAGCCTCTAATTTTAAGAGATTGTAGTGTAGCTGGATGGCTCCTCGGGAAC
CCAATGTGTGACGAATTCATCAATGTACCGGAATGGTCTTACATAGTGGAGAAGGCCAATCC
AACCAATGACCTCTGTTACCCAGGGAGTTTCAACGACTATGAAGAACTGAAACACCTATTGA
GCAGAATAAACCATTTTGAGAAAATTCAAATCATCCCCAAAAGTTCTTGGTCCGATCATGAA
GCCTCATCAGGAGTGAGCTCAGCATGTCCATACCTGGGAAGTCCCTCCTTTTTTAGAAATGT
GGTATGGCTTATCAAAAAGAACAGTACATACCCAACAATAAAGAAAAGCTACAATAATACCA
ACCAAGAAGATCTTTTGGTACTGTGGGGAATTCACCATCCTAATGATGCGGCAGAGCAGACA
AGGCTATATCAAAACCCAACCACCTATATTTCCATTGGGACATCAACACTAAACCAGAGATT
GGTACCAAAAATAGCTACTAGATCCAAAGTAAACGGGCAAAGTGGAAGGATGGAGTTCTTCT
GGACAATTTTAAAACCTAATGATGCAATCAACTTCGAGAGTAATGGAAATTTCATTGCTCCA
GAATATGCATACAAAATTGTCAAGAAAGGGGACTCAGCAATTATGAAAAGTGAATTGGAATA
TGGTAACTGCAACACCAAGTGTCAAACTCCAATGGGGGCGATAAACTCTAGTATGCCATTCC
ACAACATACACCCTCTCACCATCGGGGAATGCCCCAAATATGTGAAATCAAACAGATTAGTC
CTTGCAACAGGGCTCAGAAATAGCCCTCAAAGAGAGAGCAGAAGAAAAAAGAGAGGACTATT
TGGAGCTATAGCAGGTTTTATAGAGGGAGGATGGCAGGGAATGGTAGATGGTTGGTATGGGT
ACCACCATAGCAATGAGCAGGGGAGTGGGTACGCTGCAGACAAAGAATCCACTCAAAAGGCA
ATAGATGGAGTCACCAATAAGGTCAACTCAATCATTGACAAAATGAACACTCAGTTTGAGGC
CGTTGGAAGGGAATTTAATAACTTAGAAAGGAGAATAGAGAATTTAAACAAGAAGATGGAAG
ACGGGTTTCTAGATGTCTGGACTTATAATGCCGAACTTCTGGTTCTCATGGAAAATGAGAGA
ACTCTAGACTTTCATGACTCAAATGTTAAGAACCTCTACGACAAGGTCCGACTACAGCTTAG
GGATAATGCAAAGGAGCTGGGTAACGGTTGTTTCGAGTTCTATCACAAATGTGATAATGAAT
GTATGGAAAGTATAAGAAACGGAACGTACAACTATCCGCAGTATTCAGAAGAAGCAAGATTA
AAAAGAGAGGAAATAAGTGGGGTAAAATTGGAATCAATAGGAACTTACCAAATACTGTCAAT
TTATTCAACAGTGGCGAGTTCCCTAGCACTGGCAATCATGATGGCTGGTCTATCTTTATGGA
TGTGCTCCAATGGATCGTTACAATGCAGAATTTGCATTTAAATTTGTGAGTTCAG
Modified SARS-CoV-2 with H5 A/Indonesia/5/05 Hemagglutinin CT AA
(SEQ ID NO: 5)
MFVFLVLLPLVSSQCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLDSKTQSLLIVNNAT
NVVIKVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSY
LTPGDSSSGWTAGAAAYYVGYLQPRTELLKYNENGTITDAVDCALDPLSETKCTLKSFTVEK
GIYQTSNFRVQPTESIVRFPNITNLCPFGEVFNATRFASVYAWNRKRISNCVADYSVLYNSA
SFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGFNCYFPLQSYG
FQPTNGVGYQPYRVVVLSFELLHAPATVCGPKKSTNLVKNKCVNFNFNGLTGTGVLTESNKK
FLPFQQFGRDIADTTDAVRDPQTLEILDITPCSFGGVSVITPGTNTSNQVAVLYQDVNCTEV
PVAIHADQLTPTWRVYSTGSNVFQTRAGCLIGAEHVNNSYECDIPIGAGICASYQTQTNSPR
RARSVASQSIIAYTMSLGAENSVAYSNNSIAIPTNFTISVTTEILPVSMTKTSVDCTMYICG
DSTECSNLLLQYGSFCTQLNRALTGIAVEQDKNTQEVFAQVKQIYKTPPIKDFGGFNFSQIL
PDPSKPSKRSFIEDLLFNKVTLADAGFIKQYGDCLGDIAARDLICAQKFNGLTVLPPLLTDE
MIAQYTSALLAGTITSGWTFGAGAALQIPFAMQMAYRFNGIGVTQNVLYENQKLIANQFNSA
IGKIQDSLSSTASALGKLQDVVNQNAQALNTLVKQLSSNFGAISSVLNDILSRLDKVEAEVQ
IDRLITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINASVVN
IQKEIDRLNEVAKNLNESLIDLQELGKYEQYIKWPWYIWLGFIAGLIAIVMVTIMLSLWMCS
NGSLQCRICI
H1 A/California/7/2009 Hemagglutinin TM/CT AA
(SEQ ID NO: 6)
IDGVKLESTRIYQILAIYSTVASSLVLVVSLGAISFWMCSNGSLQCRICI
H2 A/Singapore/1/1957 Hemagglutinin TM/CT AA
(SEQ ID NO: 7)
IKGVKLSSMGVYQILAIYATVAGSLSLAIMMAGISFWMCSNGSLQCRICI
H3 A/Minnesota/41/2019 Hemagglutinin TM/CT AA
(SEQ ID NO: 8)
IKGVELKSGYKDWILWISFAISCFLLCVALLGFIMWACQKGNIRCNICI
H5 A/Indonesia/5/05 Hemagglutinin TM/CT AA
(SEQ ID NO: 9)
ISGVKLESIGTYQILSIYSTVASSLALAIMMAGLSLWMCSNGSLQCRICI
H6 A/Teal/Hong Kong/W312/97 Hemagglutinin TM/CT AA
(SEQ ID NO: 10)
IESVKLENLGVYQILAIYSTVSSSLVLVGLIMAMGLWMCSNGSMQCRICI
H7 A/Guangdong/17SF003/2016 Hemagglutinin TM/CT AA
(SEQ ID NO: 11)
IDPVKLSSGYKDVILWFSFGASCFILLAIVMGLVFICVKNGNMRCTICI
H9 A/Hong Kong/1073/99 Hemagglutinin TM/CT AA
(SEQ ID NO: 12)
IEGVKLESEGTYKILTIYSTVASSLVLAMGFAAFLFWAMSNGSCRCNICI
B/Washington/02/2019 Hemagglutinin TM/CT AA
(SEQ ID NO: 13)
AASLNDDGLDNHTILLYYSTAASSLAVTLMIAIFVVYMVSRDNVSCSICL
Consensus Sequence of C-Terminal Region of Influenza Hemagglutinin
(SEQ ID NO: 14)
IXGVKLXSXGXYXILXIYSTVASSLXLXXXXXXXXXWMCSNGSXXCXICI
Consensus Sequence of CT Domain of Influenza Hemagglutinin
(SEQ ID NO: 15)
XXWMCSNGSXXCXICI
C-Terminal Region of Native SARS-CoV-2 S protein wtTM/CT
(SEQ ID NO: 16)
WYEQYIKWPWYIWLGFIAGLIAIVMVTIMLCCMTSCCSCLKGCCSCGSCCKFDEDDSEPVLK
GVKLHYT
C-Terminal Region of H5 A/Indonesia/5/05 Hemagglutinin
(SEQ ID NO: 17)
ISGVKLESIGTYQILSIYSTVASSLALAIMMAGLSLWMCSNGSLQCRICI
C-Terminal Region of Modified SARS-CoV-2 S protein with H5i Hemagglutinin CT
(SEQ ID NO: 18)
WYIWLGFIAGLIAIVMVTIMLSLWMCSNGSLQCRICI
C-Terminal Region of Modified SARS-CoV-2 S protein with H5i Hemagglutinin CT,
Variation 1
(SEQ ID NO: 19)
WYIWLGFIAGLIAIVMVTIMMAGLSLWMCSNGSLQCRICI
(no SP) SARS-CoV-2 S protein GSAS + PP wtTM/CT AA
(SEQ ID NO: 20)
VNLTTRTQLPPAYTNSFTRGVYYPDKVFRSSVLHSTQDLFLPFFSNVTWFHAIHVSGTNGTK
RFDNPVLPFNDGVYFASTEKSNIIRGWIFGTTLDSKTQSLLIVNNATNVVIKVCEFQFCNDP
FLGVYYHKNNKSWMESEFRVYSSANNCTFEYVSQPFLMDLEGKQGNFKNLREFVFKNIDGYF
KIYSKHTPINLVRDLPQGFSALEPLVDLPIGINITRFQTLLALHRSYLTPGDSSSGWTAGAA
AYYVGYLQPRTFLLKYNENGTITDAVDCALDPLSETKCTLKSFTVEKGIYQTSNFRVQPTES
IVRFPNITNLCPFGEVFNATRFASVYAWNRKRISNCVADYSVLYNSASFSTFKCYGVSPTKL
NDLCFTNVYADSFVIRGDEVRQIAPGQTGKIADYNYKLPDDFTGCVIAWNSNNLDSKVGGNY
NYLYRLFRKSNLKPFERDISTEIYQAGSTPCNGVEGFNCYFPLQSYGFQPTNGVGYQPYRVV
VLSFELLHAPATVCGPKKSTNLVKNKCVNFNFNGLTGTGVLTESNKKFLPFQQFGRDIADTT
DAVRDPQTLEILDITPCSFGGVSVITPGTNTSNQVAVLYQDVNCTEVPVAIHADQLTPTWRV
YSTGSNVFQTRAGCLIGAEHVNNSYECDIPIGAGICASYQTQTNSPGSASSVASQSIIAYTM
SLGAENSVAYSNNSIAIPTNFTISVTTEILPVSMTKTSVDCTMYICGDSTECSNLLLQYGSF
CTQLNRALTGIAVEQDKNTQEVFAQVKQIYKTPPIKDFGGFNFSQILPDPSKPSKRSFIEDL
LFNKVTLADAGFIKQYGDCLGDIAARDLICAQKFNGLTVLPPLLTDEMIAQYTSALLAGTIT
SGWTFGAGAALQIPFAMQMAYRFNGIGVTQNVLYENQKLIANQFNSAIGKIQDSLSSTASAL
GKLQDVVNQNAQALNTLVKQLSSNFGAISSVLNDILSRLDPPEAEVQIDRLITGRLQSLQTY
VTQQLIRAAEIRASANLAATKMSECVLGQSKRVDFCGKGYHLMSFPQSAPHGVVFLHVTYVP
AQEKNFTTAPAICHDGKAHFPREGVFVSNGTHWFVTQRNFYEPQIITTDNTFVSGNCDVVIG
IVNNTVYDPLQPELDSFKEELDKYFKNHTSPDVDLGDISGINASVVNIQKEIDRLNEVAKNL
NESLIDLQELGKYEQYIKWPWYIWLGFIAGLIAIVMVTIMLCCMTSCCSCLKGCCSCGSCCK
FDEDDSEPVLKGVKLHYT
(no SP) Modified SARS-CoV-2 S protein GSAS + PP with H5i Hemagglutinin CT AA
(SEQ ID NO: 21)
VNLTTRTQLPPAYTNSFTRGVYYPDKVFRSSVLHSTQDLFLPFFSNVTWFHAIHVSGTNGTK
RFDNPVLPFNDGVYFASTEKSNIIRGWIFGTTLDSKTQSLLIVNNATNVVIKVCEFQFCNDP
FLGVYYHKNNKSWMESEFRVYSSANNCTFEYVSQPFLMDLEGKQGNFKNLREFVFKNIDGYF
KIYSKHTPINLVRDLPQGFSALEPLVDLPIGINITRFQTLLALHRSYLTPGDSSSGWTAGAA
AYYVGYLQPRTFLLKYNENGTITDAVDCALDPLSETKCTLKSFTVEKGIYQTSNFRVQPTES
IVRFPNITNLCPFGEVFNATRFASVYAWNRKRISNCVADYSVLYNSASFSTFKCYGVSPTKL
NDLCFTNVYADSFVIRGDEVRQIAPGQTGKIADYNYKLPDDFTGCVIAWNSNNLDSKVGGNY
NYLYRLFRKSNLKPFERDISTEIYQAGSTPCNGVEGFNCYFPLQSYGFQPTNGVGYQPYRVV
VLSFELLHAPATVCGPKKSTNLVKNKCVNFNFNGLTGTGVLTESNKKFLPFQQFGRDIADTT
DAVRDPQTLEILDITPCSFGGVSVITPGTNTSNQVAVLYQDVNCTEVPVAIHADQLTPTWRV
YSTGSNVFQTRAGCLIGAEHVNNSYECDIPIGAGICASYQTQTNSPGSASSVASQSIIAYTM
SLGAENSVAYSNNSIAIPTNFTISVTTEILPVSMTKTSVDCTMYICGDSTECSNLLLQYGSF
CTQLNRALTGIAVEQDKNTQEVFAQVKQIYKTPPIKDFGGFNFSQILPDPSKPSKRSFIEDL
LFNKVTLADAGFIKQYGDCLGDIAARDLICAQKFNGLTVLPPLLTDEMIAQYTSALLAGTIT
SGWTFGAGAALQIPFAMQMAYRFNGIGVTQNVLYENQKLIANQFNSAIGKIQDSLSSTASAL
GKLQDVVNQNAQALNTLVKQLSSNFGAISSVLNDILSRLDPPEAEVQIDRLITGRLQSLQTY
VTQQLIRAAEIRASANLAATKMSECVLGQSKRVDFCGKGYHLMSFPQSAPHGVVFLHVTYVP
AQEKNFTTAPAICHDGKAHFPREGVFVSNGTHWFVTQRNFYEPQIITTDNTFVSGNCDVVIG
IVNNTVYDPLQPELDSFKEELDKYFKNHTSPDVDLGDISGINASVVNIQKEIDRLNEVAKNL
NESLIDLQELGKYEQYIKWPWYIWLGFIAGLIAIVMVTIMLSLWMCSNGSLQCRICI
PDI-SARS-CoV-2 S protein GSAS + PP wtTM/CT-DNA
(SEQ ID NO: 22)
ATGGCGAAAAACGTTGCGATTTTCGGCTTATTGTTTTCTCTTCTTGTGTTGGTTCCTTCTCA
GATCTTCGCGGTGAATCTTACGACGCGAACACAGTTACCACCCGCATATACAAATAGCTTCA
CTCGGGGTGTTTATTACCCCGACAAAGTGTTCAGGTCCTCCGTGCTCCACTCAACACAGGAC
CTCTTTCTTCCTTTCTTTTCTAACGTGACATGGTTTCATGCCATTCATGTATCCGGCACTAA
CGGTACTAAGAGGTTCGATAATCCTGTGCTCCCTTTCAATGACGGCGTTTACTTTGCAAGCA
CAGAGAAGAGTAACATCATCCGAGGTTGGATCTTTGGCACTACCCTCGATTCAAAGACGCAG
AGCCTCCTCATTGTGAACAATGCCACTAACGTGGTGATCAAAGTTTGCGAGTTTCAGTTCTG
CAATGACCCTTTCTTGGGGGTGTACTATCATAAGAACAACAAGTCTTGGATGGAATCTGAAT
TCCGCGTCTATAGCAGCGCCAACAACTGCACCTTTGAATACGTGTCCCAGCCCTTCCTTATG
GACCTGGAGGGAAAGCAGGGAAACTTTAAGAATCTGAGAGAGTTCGTGTTTAAAAATATCGA
CGGCTATTTTAAGATCTATTCTAAGCACACGCCTATTAATCTCGTGCGCGATCTTCCACAAG
GCTTCAGCGCCCTGGAACCACTCGTGGACCTCCCAATTGGTATCAACATCACTAGATTTCAG
ACTCTGCTTGCCCTCCACCGATCCTATCTGACACCCGGAGACTCCTCTAGCGGCTGGACTGC
CGGCGCTGCCGCTTATTACGTTGGTTATCTTCAGCCACGCACGTTCCTGCTGAAGTATAACG
AGAATGGTACTATTACCGATGCCGTGGATTGTGCCCTTGACCCCCTGTCCGAAACTAAGTGC
ACACTCAAGTCATTCACTGTGGAAAAAGGAATCTACCAGACAAGCAATTTTCGGGTCCAGCC
TACTGAGAGCATTGTGCGCTTTCCTAACATCACAAATCTTTGCCCCTTCGGAGAGGTTTTCA
ATGCTACACGGTTTGCCTCCGTGTATGCCTGGAACCGCAAGAGAATTTCCAATTGCGTGGCC
GATTACTCCGTGCTCTACAATAGTGCAAGCTTTAGCACCTTTAAGTGCTATGGCGTATCCCC
TACTAAGCTTAACGACTTGTGTTTCACAAACGTGTATGCCGACTCCTTTGTGATACGGGGCG
ACGAAGTTAGACAGATAGCACCAGGACAGACGGGAAAGATAGCTGACTACAACTATAAGCTT
CCTGATGACTTCACTGGCTGCGTTATCGCGTGGAATTCTAACAACCTGGACTCAAAAGTCGG
CGGCAACTATAACTATCTCTATCGGCTGTTCCGCAAGAGTAACCTTAAGCCCTTTGAGAGAG
ATATAAGCACTGAAATCTACCAGGCTGGCAGTACGCCCTGTAATGGCGTGGAAGGCTTTAAT
TGTTATTTTCCACTGCAATCCTATGGTTTTCAGCCAACCAATGGCGTGGGCTACCAACCATA
CCGCGTCGTGGTGCTCTCCTTTGAACTGCTCCACGCTCCCGCGACTGTCTGCGGCCCCAAGA
AGTCCACGAACCTTGTGAAGAATAAGTGCGTTAATTTTAATTTCAACGGCCTCACTGGAACA
GGAGTGCTCACTGAGAGTAACAAGAAGTTCCTGCCATTTCAACAATTTGGCAGAGACATAGC
CGATACTACTGACGCCGTTAGGGACCCCCAGACCCTCGAGATTCTCGATATAACGCCCTGCT
CCTTCGGTGGAGTTTCCGTGATCACGCCAGGCACCAATACCAGTAACCAGGTCGCCGTGCTG
TATCAGGATGTCAACTGTACTGAGGTGCCCGTAGCCATCCATGCGGATCAGCTCACACCAAC
TTGGAGGGTGTACAGCACCGGCTCCAATGTATTCCAGACTCGGGCCGGATGCCTTATTGGCG
CCGAACACGTGAACAATAGTTACGAATGCGATATTCCAATTGGCGCCGGAATCTGTGCTAGC
TACCAGACTCAGACGAACTCCCCAGGCAGCGCCAGCAGCGTTGCCAGCCAGTCAATCATCGC
TTATACAATGTCACTTGGAGCCGAAAACTCCGTGGCTTACTCAAACAACAGCATCGCCATCC
CCACAAACTTCACCATATCCGTGACAACTGAGATTCTGCCAGTGTCCATGACTAAGACGTCC
GTAGATTGCACTATGTACATATGCGGCGACAGCACAGAATGTTCTAATCTGCTGCTGCAATA
TGGAAGCTTCTGCACTCAACTGAACAGAGCGCTCACAGGCATCGCCGTGGAGCAGGATAAGA
ATACCCAGGAGGTGTTCGCCCAAGTTAAGCAGATCTACAAGACCCCACCCATAAAGGATTTC
GGTGGATTCAATTTTAGTCAGATACTCCCAGACCCATCTAAGCCATCCAAGAGGAGCTTTAT
CGAGGATCTTTTGTTTAACAAAGTTACTCTGGCCGACGCCGGTTTCATCAAGCAGTACGGAG
ATTGCCTCGGCGACATCGCTGCTCGTGACCTCATCTGTGCGCAAAAGTTTAACGGTCTGACG
GTGCTGCCTCCCCTCCTTACTGATGAAATGATCGCCCAGTATACCAGCGCACTCCTCGCTGG
CACCATAACATCCGGTTGGACATTCGGCGCTGGTGCAGCACTGCAGATACCATTCGCCATGC
AAATGGCATATCGTTTCAACGGTATCGGTGTCACACAGAATGTCCTATATGAGAACCAGAAG
CTGATCGCAAATCAGTTCAATAGTGCCATCGGAAAAATCCAGGATAGCCTTAGCAGCACAGC
CTCAGCCCTTGGCAAACTCCAGGATGTCGTGAACCAGAATGCCCAGGCTCTCAATACCCTCG
TGAAGCAGCTCTCATCTAATTTCGGCGCAATTTCCAGTGTCCTCAACGACATCCTCAGCCGC
CTCGACCCCCCCGAGGCCGAAGTGCAGATTGACAGACTGATTACAGGTCGACTCCAGAGCCT
CCAGACTTACGTGACTCAGCAGCTGATAAGAGCCGCCGAGATAAGGGCCAGCGCTAACCTGG
CTGCCACAAAGATGTCTGAGTGCGTGCTGGGCCAGTCCAAGAGAGTAGACTTCTGTGGCAAA
GGCTACCATCTGATGAGCTTCCCACAATCCGCACCTCACGGCGTAGTGTTCCTCCACGTGAC
ATATGTACCGGCTCAGGAGAAGAATTTCACTACCGCTCCTGCTATATGCCATGATGGAAAGG
CTCACTTCCCCCGGGAGGGGGTGTTCGTGTCCAACGGCACCCATTGGTTTGTGACTCAGCGG
AATTTCTACGAACCCCAGATCATAACCACTGACAACACATTTGTGTCCGGAAATTGTGACGT
GGTCATTGGAATAGTGAACAACACTGTTTATGATCCACTGCAGCCAGAACTTGACAGCTTTA
AGGAGGAGCTCGACAAGTACTTCAAGAATCATACGTCACCAGATGTGGACCTCGGAGATATT
AGCGGTATCAATGCCAGTGTTGTCAATATTCAGAAGGAAATAGACCGCCTTAATGAGGTCGC
CAAAAATCTGAACGAGAGCCTCATCGATCTTCAGGAGCTGGGCAAATATGAGCAGTACATCA
AGTGGCCTTGGTATATTTGGCTTGGCTTCATCGCCGGCCTGATCGCCATAGTAATGGTCACA
ATTATGCTCTGCTGCATGACCTCTTGCTGCTCCTGTCTGAAAGGCTGCTGCTCTTGCGGATC
CTGCTGCAAATTTGATGAGGATGACAGTGAACCAGTCCTGAAGGGCGTGAAGCTGCACTATA
CTTAG
PDI-SARS-CoV-2 S protein GSAS + PP wtTM/CT-AA
(SEQ ID NO: 23)
MAKNVAIFGLLFSLLVLVPSQIFAVNLTTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANNCTFEYVSQPFLM
DLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSETKC
TLKSFTVEKGIYQTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQAGSTPCNGVEGFN
CYFPLQSYGFQPTNGVGYQPYRVVVLSFELLHAPATVCGPKKSTNLVKNKCVNFNFNGLTGT
GVLTESNKKFLPFQQFGRDIADTTDAVRDPQTLEILDITPCSFGGVSVITPGTNTSNQVAVL
YQDVNCTEVPVAIHADQLTPTWRVYSTGSNVFQTRAGCLIGAEHVNNSYECDIPIGAGICAS
YQTQTNSPGSASSVASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQVKQIYKTPPIKDF
GGFNFSQILPDPSKPSKRSFIEDLLFNKVTLADAGFIKQYGDCLGDIAARDLICAQKFNGLT
VLPPLLTDEMIAQYTSALLAGTITSGWTFGAGAALQIPFAMQMAYRFNGIGVTQNVLYENQK
LIANQFNSAIGKIQDSLSSTASALGKLQDVVNQNAQALNTLVKQLSSNFGAISSVLNDILSR
LDPPEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKYFKNHTSPDVDLGDI
SGINASVVNIQKEIDRLNEVAKNLNESLIDLQELGKYEQYIKWPWYIWLGFIAGLIAIVMVT
IMLCCMTSCCSCLKGCCSCGSCCKFDEDDSEPVLKGVKLHYT
IF(PDI)-CoV(opt2).c
(SEQ ID NO: 24)
TCTCAGATCTTCGCGGTGAATCTTACGACGCGAACACAGTTACCACCCGCAT
IF(AVB)-CoV(opt2).r
(SEQ ID NO: 25)
ACGACACGACTAAGGCCTCTAAGTATAGTGCAGCTTCACGCCCTTCAGGAC
PDI-Modified SARS-CoV-2 S protein GSAS + PP H5iTM/CT-DNA
(SEQ ID NO: 26)
ATGGCGAAAAACGTTGCGATTTTCGGCTTATTGTTTTCTCTTCTTGTGTTGGTTCCTTCTCA
GATCTTCGCGGTGAATCTTACGACGCGAACACAGTTACCACCCGCATATACAAATAGCTTCA
CTCGGGGTGTTTATTACCCCGACAAAGTGTTCAGGTCCTCCGTGCTCCACTCAACACAGGAC
CTCTTTCTTCCTTTCTTTTCTAACGTGACATGGTTTCATGCCATTCATGTATCCGGCACTAA
CGGTACTAAGAGGTTCGATAATCCTGTGCTCCCTTTCAATGACGGCGTTTACTTTGCAAGCA
CAGAGAAGAGTAACATCATCCGAGGTTGGATCTTTGGCACTACCCTCGATTCAAAGACGCAG
AGCCTCCTCATTGTGAACAATGCCACTAACGTGGTGATCAAAGTTTGCGAGTTTCAGTTCTG
CAATGACCCTTTCTTGGGGGTGTACTATCATAAGAACAACAAGTCTTGGATGGAATCTGAAT
TCCGCGTCTATAGCAGCGCCAACAACTGCACCTTTGAATACGTGTCCCAGCCCTTCCTTATG
GACCTGGAGGGAAAGCAGGGAAACTTTAAGAATCTGAGAGAGTTCGTGTTTAAAAATATCGA
CGGCTATTTTAAGATCTATTCTAAGCACACGCCTATTAATCTCGTGCGCGATCTTCCACAAG
GCTTCAGCGCCCTGGAACCACTCGTGGACCTCCCAATTGGTATCAACATCACTAGATTTCAG
ACTCTGCTTGCCCTCCACCGATCCTATCTGACACCCGGAGACTCCTCTAGCGGCTGGACTGC
CGGCGCTGCCGCTTATTACGTTGGTTATCTTCAGCCACGCACGTTCCTGCTGAAGTATAACG
AGAATGGTACTATTACCGATGCCGTGGATTGTGCCCTTGACCCCCTGTCCGAAACTAAGTGC
ACACTCAAGTCATTCACTGTGGAAAAAGGAATCTACCAGACAAGCAATTTTCGGGTCCAGCC
TACTGAGAGCATTGTGCGCTTTCCTAACATCACAAATCTTTGCCCCTTCGGAGAGGTTTTCA
ATGCTACACGGTTTGCCTCCGTGTATGCCTGGAACCGCAAGAGAATTTCCAATTGCGTGGCC
GATTACTCCGTGCTCTACAATAGTGCAAGCTTTAGCACCTTTAAGTGCTATGGCGTATCCCC
TACTAAGCTTAACGACTTGTGTTTCACAAACGTGTATGCCGACTCCTTTGTGATACGGGGCG
ACGAAGTTAGACAGATAGCACCAGGACAGACGGGAAAGATAGCTGACTACAACTATAAGCTT
CCTGATGACTTCACTGGCTGCGTTATCGCGTGGAATTCTAACAACCTGGACTCAAAAGTCGG
CGGCAACTATAACTATCTCTATCGGCTGTTCCGCAAGAGTAACCTTAAGCCCTTTGAGAGAG
ATATAAGCACTGAAATCTACCAGGCTGGCAGTACGCCCTGTAATGGCGTGGAAGGCTTTAAT
TGTTATTTTCCACTGCAATCCTATGGTTTTCAGCCAACCAATGGCGTGGGCTACCAACCATA
CCGCGTCGTGGTGCTCTCCTTTGAACTGCTCCACGCTCCCGCGACTGTCTGCGGCCCCAAGA
AGTCCACGAACCTTGTGAAGAATAAGTGCGTTAATTTTAATTTCAACGGCCTCACTGGAACA
GGAGTGCTCACTGAGAGTAACAAGAAGTTCCTGCCATTTCAACAATTTGGCAGAGACATAGC
CGATACTACTGACGCCGTTAGGGACCCCCAGACCCTCGAGATTCTCGATATAACGCCCTGCT
CCTTCGGTGGAGTTTCCGTGATCACGCCAGGCACCAATACCAGTAACCAGGTCGCCGTGCTG
TATCAGGATGTCAACTGTACTGAGGTGCCCGTAGCCATCCATGCGGATCAGCTCACACCAAC
TTGGAGGGTGTACAGCACCGGCTCCAATGTATTCCAGACTCGGGCCGGATGCCTTATTGGCG
CCGAACACGTGAACAATAGTTACGAATGCGATATTCCAATTGGCGCCGGAATCTGTGCTAGC
TACCAGACTCAGACGAACTCCCCAGGCAGCGCCAGCAGCGTTGCCAGCCAGTCAATCATCGC
TTATACAATGTCACTTGGAGCCGAAAACTCCGTGGCTTACTCAAACAACAGCATCGCCATCC
CCACAAACTTCACCATATCCGTGACAACTGAGATTCTGCCAGTGTCCATGACTAAGACGTCC
GTAGATTGCACTATGTACATATGCGGCGACAGCACAGAATGTTCTAATCTGCTGCTGCAATA
TGGAAGCTTCTGCACTCAACTGAACAGAGCGCTCACAGGCATCGCCGTGGAGCAGGATAAGA
ATACCCAGGAGGTGTTCGCCCAAGTTAAGCAGATCTACAAGACCCCACCCATAAAGGATTTC
GGTGGATTCAATTTTAGTCAGATACTCCCAGACCCATCTAAGCCATCCAAGAGGAGCTTTAT
CGAGGATCTTTTGTTTAACAAAGTTACTCTGGCCGACGCCGGTTTCATCAAGCAGTACGGAG
ATTGCCTCGGCGACATCGCTGCTCGTGACCTCATCTGTGCGCAAAAGTTTAACGGTCTGACG
GTGCTGCCTCCCCTCCTTACTGATGAAATGATCGCCCAGTATACCAGCGCACTCCTCGCTGG
CACCATAACATCCGGTTGGACATTCGGCGCTGGTGCAGCACTGCAGATACCATTCGCCATGC
AAATGGCATATCGTTTCAACGGTATCGGTGTCACACAGAATGTCCTATATGAGAACCAGAAG
CTGATCGCAAATCAGTTCAATAGTGCCATCGGAAAAATCCAGGATAGCCTTAGCAGCACAGC
CTCAGCCCTTGGCAAACTCCAGGATGTCGTGAACCAGAATGCCCAGGCTCTCAATACCCTCG
TGAAGCAGCTCTCATCTAATTTCGGCGCAATTTCCAGTGTCCTCAACGACATCCTCAGCCGC
CTCGACCCCCCCGAGGCCGAAGTGCAGATTGACAGACTGATTACAGGTCGACTCCAGAGCCT
CCAGACTTACGTGACTCAGCAGCTGATAAGAGCCGCCGAGATAAGGGCCAGCGCTAACCTGG
CTGCCACAAAGATGTCTGAGTGCGTGCTGGGCCAGTCCAAGAGAGTAGACTTCTGTGGCAAA
GGCTACCATCTGATGAGCTTCCCACAATCCGCACCTCACGGCGTAGTGTTCCTCCACGTGAC
ATATGTACCGGCTCAGGAGAAGAATTTCACTACCGCTCCTGCTATATGCCATGATGGAAAGG
CTCACTTCCCCCGGGAGGGGGTGTTCGTGTCCAACGGCACCCATTGGTTTGTGACTCAGCGG
AATTTCTACGAACCCCAGATCATAACCACTGACAACACATTTGTGTCCGGAAATTGTGACGT
GGTCATTGGAATAGTGAACAACACTGTTTATGATCCACTGCAGCCAGAACTTGACAGCTTTA
AGGAGGAGCTCGACAAGTACTTCAAGAATCATACGTCACCAGATGTGGACCTCGGAGATATT
AGCGGTATCAATGCCAGTGTTGTCAATATTCAGAAGGAAATAGACCGCCTTAATGAGGTCGC
CAAAAATCTGAACGAGAGCCTCATCGATCTTCAGGAGCTGGGCAAATATGAGCAGTACATCA
AGTGGCCTTGGTATCAAATACTGTCAATTTATTCAACAGTGGCGAGTTCCCTAGCACTGGCA
ATCATGATGGCTGGTCTATCTTTATGGATGTGCTCCAATGGATCGTTACAATGCAGAATTTG
CATTTAA
PDI-Modified SARS-CoV-2 S protein GSAS + PP H5iTM/CT-AA
(SEQ ID NO: 27)
MAKNVAIFGLLFSLLVLVPSQIFAVNLTTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANNCTFEYVSQPFLM
DLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSETKC
TLKSFTVEKGIYQTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQAGSTPCNGVEGFN
CYFPLQSYGFQPTNGVGYQPYRVVVLSFELLHAPATVCGPKKSTNLVKNKCVNFNFNGLTGT
GVLTESNKKFLPFQQFGRDIADTTDAVRDPQTLEILDITPCSFGGVSVITPGTNTSNQVAVL
YQDVNCTEVPVAIHADQLTPTWRVYSTGSNVFQTRAGCLIGAEHVNNSYECDIPIGAGICAS
YQTQTNSPGSASSVASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQVKQIYKTPPIKDF
GGFNFSQILPDPSKPSKRSFIEDLLFNKVTLADAGFIKQYGDCLGDIAARDLICAQKFNGLT
VLPPLLTDEMIAQYTSALLAGTITSGWTFGAGAALQIPFAMQMAYRFNGIGVTQNVLYENQK
LIANQFNSAIGKIQDSLSSTASALGKLQDVVNQNAQALNTLVKQLSSNFGAISSVLNDILSR
LDPPEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKYFKNHTSPDVDLGDI
SGINASVVNIQKEIDRLNEVAKNLNESLIDLQELGKYEQYIKWPWYQILSIYSTVASSLALA
IMMAGLSLWMCSNGSLQCRICI
IF(Avb)-H5I.r
(SEQ ID NO: 28)
ACGACACGACTAAGGCCTTTAAATGCAAATTCTGCATTGTAACGATCC
PDI-Modified SARS-CoV-2 S protein GSAS + PP wtTM/H5iCT-DNA
(SEQ ID NO: 29)
ATGGCGAAAAACGTTGCGATTTTCGGCTTATTGTTTTCTCTTCTTGTGTTGGTTCCTTCTCA
GATCTTCGCGGTGAATCTTACGACGCGAACACAGTTACCACCCGCATATACAAATAGCTTCA
CTCGGGGTGTTTATTACCCCGACAAAGTGTTCAGGTCCTCCGTGCTCCACTCAACACAGGAC
CTCTTTCTTCCTTTCTTTTCTAACGTGACATGGTTTCATGCCATTCATGTATCCGGCACTAA
CGGTACTAAGAGGTTCGATAATCCTGTGCTCCCTTTCAATGACGGCGTTTACTTTGCAAGCA
CAGAGAAGAGTAACATCATCCGAGGTTGGATCTTTGGCACTACCCTCGATTCAAAGACGCAG
AGCCTCCTCATTGTGAACAATGCCACTAACGTGGTGATCAAAGTTTGCGAGTTTCAGTTCTG
CAATGACCCTTTCTTGGGGGTGTACTATCATAAGAACAACAAGTCTTGGATGGAATCTGAAT
TCCGCGTCTATAGCAGCGCCAACAACTGCACCTTTGAATACGTGTCCCAGCCCTTCCTTATG
GACCTGGAGGGAAAGCAGGGAAACTTTAAGAATCTGAGAGAGTTCGTGTTTAAAAATATCGA
CGGCTATTTTAAGATCTATTCTAAGCACACGCCTATTAATCTCGTGCGCGATCTTCCACAAG
GCTTCAGCGCCCTGGAACCACTCGTGGACCTCCCAATTGGTATCAACATCACTAGATTTCAG
ACTCTGCTTGCCCTCCACCGATCCTATCTGACACCCGGAGACTCCTCTAGCGGCTGGACTGC
CGGCGCTGCCGCTTATTACGTTGGTTATCTTCAGCCACGCACGTTCCTGCTGAAGTATAACG
AGAATGGTACTATTACCGATGCCGTGGATTGTGCCCTTGACCCCCTGTCCGAAACTAAGTGC
ACACTCAAGTCATTCACTGTGGAAAAAGGAATCTACCAGACAAGCAATTTTCGGGTCCAGCC
TACTGAGAGCATTGTGCGCTTTCCTAACATCACAAATCTTTGCCCCTTCGGAGAGGTTTTCA
ATGCTACACGGTTTGCCTCCGTGTATGCCTGGAACCGCAAGAGAATTTCCAATTGCGTGGCC
GATTACTCCGTGCTCTACAATAGTGCAAGCTTTAGCACCTTTAAGTGCTATGGCGTATCCCC
TACTAAGCTTAACGACTTGTGTTTCACAAACGTGTATGCCGACTCCTTTGTGATACGGGGCG
ACGAAGTTAGACAGATAGCACCAGGACAGACGGGAAAGATAGCTGACTACAACTATAAGCTT
CCTGATGACTTCACTGGCTGCGTTATCGCGTGGAATTCTAACAACCTGGACTCAAAAGTCGG
CGGCAACTATAACTATCTCTATCGGCTGTTCCGCAAGAGTAACCTTAAGCCCTTTGAGAGAG
ATATAAGCACTGAAATCTACCAGGCTGGCAGTACGCCCTGTAATGGCGTGGAAGGCTTTAAT
TGTTATTTTCCACTGCAATCCTATGGTTTTCAGCCAACCAATGGCGTGGGCTACCAACCATA
CCGCGTCGTGGTGCTCTCCTTTGAACTGCTCCACGCTCCCGCGACTGTCTGCGGCCCCAAGA
AGTCCACGAACCTTGTGAAGAATAAGTGCGTTAATTTTAATTTCAACGGCCTCACTGGAACA
GGAGTGCTCACTGAGAGTAACAAGAAGTTCCTGCCATTTCAACAATTTGGCAGAGACATAGC
CGATACTACTGACGCCGTTAGGGACCCCCAGACCCTCGAGATTCTCGATATAACGCCCTGCT
CCTTCGGTGGAGTTTCCGTGATCACGCCAGGCACCAATACCAGTAACCAGGTCGCCGTGCTG
TATCAGGATGTCAACTGTACTGAGGTGCCCGTAGCCATCCATGCGGATCAGCTCACACCAAC
TTGGAGGGTGTACAGCACCGGCTCCAATGTATTCCAGACTCGGGCCGGATGCCTTATTGGCG
CCGAACACGTGAACAATAGTTACGAATGCGATATTCCAATTGGCGCCGGAATCTGTGCTAGC
TACCAGACTCAGACGAACTCCCCAGGCAGCGCCAGCAGCGTTGCCAGCCAGTCAATCATCGC
TTATACAATGTCACTTGGAGCCGAAAACTCCGTGGCTTACTCAAACAACAGCATCGCCATCC
CCACAAACTTCACCATATCCGTGACAACTGAGATTCTGCCAGTGTCCATGACTAAGACGTCC
GTAGATTGCACTATGTACATATGCGGCGACAGCACAGAATGTTCTAATCTGCTGCTGCAATA
TGGAAGCTTCTGCACTCAACTGAACAGAGCGCTCACAGGCATCGCCGTGGAGCAGGATAAGA
ATACCCAGGAGGTGTTCGCCCAAGTTAAGCAGATCTACAAGACCCCACCCATAAAGGATTTC
GGTGGATTCAATTTTAGTCAGATACTCCCAGACCCATCTAAGCCATCCAAGAGGAGCTTTAT
CGAGGATCTTTTGTTTAACAAAGTTACTCTGGCCGACGCCGGTTTCATCAAGCAGTACGGAG
ATTGCCTCGGCGACATCGCTGCTCGTGACCTCATCTGTGCGCAAAAGTTTAACGGTCTGACG
GTGCTGCCTCCCCTCCTTACTGATGAAATGATCGCCCAGTATACCAGCGCACTCCTCGCTGG
CACCATAACATCCGGTTGGACATTCGGCGCTGGTGCAGCACTGCAGATACCATTCGCCATGC
AAATGGCATATCGTTTCAACGGTATCGGTGTCACACAGAATGTCCTATATGAGAACCAGAAG
CTGATCGCAAATCAGTTCAATAGTGCCATCGGAAAAATCCAGGATAGCCTTAGCAGCACAGC
CTCAGCCCTTGGCAAACTCCAGGATGTCGTGAACCAGAATGCCCAGGCTCTCAATACCCTCG
TGAAGCAGCTCTCATCTAATTTCGGCGCAATTTCCAGTGTCCTCAACGACATCCTCAGCCGC
CTCGACCCCCCCGAGGCCGAAGTGCAGATTGACAGACTGATTACAGGTCGACTCCAGAGCCT
CCAGACTTACGTGACTCAGCAGCTGATAAGAGCCGCCGAGATAAGGGCCAGCGCTAACCTGG
CTGCCACAAAGATGTCTGAGTGCGTGCTGGGCCAGTCCAAGAGAGTAGACTTCTGTGGCAAA
GGCTACCATCTGATGAGCTTCCCACAATCCGCACCTCACGGCGTAGTGTTCCTCCACGTGAC
ATATGTACCGGCTCAGGAGAAGAATTTCACTACCGCTCCTGCTATATGCCATGATGGAAAGG
CTCACTTCCCCCGGGAGGGGGTGTTCGTGTCCAACGGCACCCATTGGTTTGTGACTCAGCGG
AATTTCTACGAACCCCAGATCATAACCACTGACAACACATTTGTGTCCGGAAATTGTGACGT
GGTCATTGGAATAGTGAACAACACTGTTTATGATCCACTGCAGCCAGAACTTGACAGCTTTA
AGGAGGAGCTCGACAAGTACTTCAAGAATCATACGTCACCAGATGTGGACCTCGGAGATATT
AGCGGTATCAATGCCAGTGTTGTCAATATTCAGAAGGAAATAGACCGCCTTAATGAGGTCGC
CAAAAATCTGAACGAGAGCCTCATCGATCTTCAGGAGCTGGGCAAATATGAGCAGTACATCA
AGTGGCCTTGGTATATTTGGCTTGGCTTCATCGCCGGCCTGATCGCCATAGTAATGGTCACA
ATTATGCTCTCTTTATGGATGTGCTCCAATGGATCGTTACAATGCAGAATTTGCATTTAA
PDI-Modified SARS-CoV-2 S protein GSAS + PP wtTM/H5iCT-AA
(SEQ ID NO: 30)
MAKNVAIFGLLFSLLVLVPSQIFAVNLTTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANNCTFEYVSQPFLM
DLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSETKC
TLKSFTVEKGIYQTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQAGSTPCNGVEGFN
CYFPLQSYGFQPTNGVGYQPYRVVVLSFELLHAPATVCGPKKSTNLVKNKCVNFNFNGLTGT
GVLTESNKKFLPFQQFGRDIADTTDAVRDPQTLEILDITPCSFGGVSVITPGTNTSNQVAVL
YQDVNCTEVPVAIHADQLTPTWRVYSTGSNVFQTRAGCLIGAEHVNNSYECDIPIGAGICAS
YQTQTNSPGSASSVASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQVKQIYKTPPIKDF
GGFNFSQILPDPSKPSKRSFIEDLLFNKVTLADAGFIKQYGDCLGDIAARDLICAQKFNGLT
VLPPLLTDEMIAQYTSALLAGTITSGWTFGAGAALQIPFAMQMAYRFNGIGVTQNVLYENQK
LIANQFNSAIGKIQDSLSSTASALGKLQDVVNQNAQALNTLVKQLSSNFGAISSVLNDILSR
LDPPEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKYFKNHTSPDVDLGDI
SGINASVVNIQKEIDRLNEVAKNLNESLIDLQELGKYEQYIKWPWYIWLGFIAGLIAIVMVT
IMLSLWMCSNGSLQCRICI
Cloning vector 8501 from left to right T-DNA
(SEQ ID NO: 31)
TGGCAGGATATATTGTGGTGTAAACAAATTGACGCTTAGACAACTTAATAACACATTGCGGA
CGTTTTTAATGTACTGAATTAACGCCGAATCCCGGGCTGGTATATTTATATGTTGTCAAATA
ACTCAAAAACCATAAAAGTTTAAGTTAGCAAGTGTGTACATTTTTACTTGAACAAAAATATT
CACCTACTACTGTTATAAATCATTATTAAACATTAGAGTAAAGAAATATGGATGATAAGAAC
AAGAGTAGTGATATTTTGACAACAATTTTGTTGCAACATTTGAGAAAATTTTGTTGTTCTCT
CTTTTCATTGGTCAAAAACAATAGAGAGAGAAAAAGGAAGAGGGAGAATAAAAACATAATGT
GAGTATGAGAGAGAAAGTTGTACAAAAGTTGTACCAAAATAGTTGTACAAATATCATTGAGG
AATTTGACAAAAGCTACACAAATAAGGGTTAATTGCTGTAAATAAATAAGGATGACGCATTA
GAGAGATGTACCATTAGAGAATTTTTGGCAAGTCATTAAAAAGAAAGAATAAATTATTTTTA
AAATTAAAAGTTGAGTCATTTGATTAAACATGTGATTATTTAATGAATTGATGAAAGAGTTG
GATTAAAGTTGTATTAGTAATTAGAATTTGGTGTCAAATTTAATTTGACATTTGATCTTTTC
CTATATATTGCCCCATAGAGTCAGTTAACTCATTTTTATATTTCATAGATCAAATAAGAGAA
ATAACGGTATATTAATCCCTCCAAAAAAAAAAAACGGTATATTTACTAAAAAATCTAAGCCA
CGTAGGAGGATAACAGGATCCCCGTAGGAGGATAACATCCAATCCAACCAATCACAACAATC
CTGATGAGATAACCCACTTTAAGCCCACGCATCTGTGGCACATCTACATTATCTAAATCACA
CATTCTTCCACACATCTGAGCCACACAAAAACCAATCCACATCTTTATCACCCATTCTATAA
AAAATCACACTTTGTGAGTCTACACTTTGATTCCCTTCAAACACATACAAAGAGAAGAGACT
AATTAATTAATTAATCATCTTGAGAGAAAATGGAACGAGCTATACAAGGAAACGACGCTAGG
GAACAAGCTAACAGTGAACGTTGGGATGGAGGATCAGGAGGTACCACTTCTCCCTTCAAACT
TCCTGACGAAAGTCCGAGTTGGACTGAGTGGCGGCTACATAACGATGAGACGAATTCGAATC
AAGATAATCCCCTTGGTTTCAAGGAAAGCTGGGGTTTCGGGAAAGTTGTATTTAAGAGATAT
CTCAGATACGACAGGACGGAAGCTTCACTGCACAGAGTCCTTGGATCTTGGACGGGAGATTC
GGTTAACTATGCAGCATCTCGATTTTTCGGTTTCGACCAGATCGGATGTACCTATAGTATTC
GGTTTCGAGGAGTTAGTATCACCGTTTCTGGAGGGTCGCGAACTCTTCAGCATCTCTGTGAG
ATGGCAATTCGGTCTAAGCAAGAACTGCTACAGCTTGCCCCAATCGAAGTGGAAAGTAATGT
ATCAAGAGGATGCCCTGAAGGTACTCAAACCTTCGAAAAAGAAAGCGAGTAAGTTAAAATGC
TTCTTCGTCTCCTATTTATAATATGGTTTGTTATTGTTAATTTTGTTCTTGTAGAAGAGCTT
AATTAATCGTTGTTGTTATGAAATACTATTTGTATGAGATGAACTGGTGTAATGTAATTCAT
TTACATAAGTGGAGTCAGAATCAGAATGTTTCCTCCATAACTAACTAGACATGAAGACCTGC
CGCGTACAATTGTCTTATATTTGAACAACTAAAATTGAACATCTTTTGCCACAACTTTATAA
GTGGTTAATATAGCTCAAATATATGGTCAAGTTCAATAGATTAATAATGGAAATATCAGTTA
TCGAAATTCATTAACAATCAACTTAACGTTATTAACTACTAATTTTATATCATCCCCTTTGA
TAAATGATAGTACACCAATTAGGAAGGAGCATGCTCGCCTAGGAGATTGTCGTTTCCCGCCT
TCAGTTTGCAAGCTGCTCTAGCCGTGTAGCCAATACGCAAACCGCCTCTCCCCGCGCGTTGG
GAATTACTAGCGCGTGTCGACAAGCTTGCATGCCGGTCAACATGGTGGAGCACGACACACTT
GTCTACTCCAAAAATATCAAAGATACAGTCTCAGAAGACCAAAGGGCAATTGAGACTTTTCA
ACAAAGGGTAATATCCGGAAACCTCCTCGGATTCCATTGCCCAGCTATCTGTCACTTTATTG
TGAAGATAGTGGAAAAGGAAGGTGGCTCCTACAAATGCCATCATTGCGATAAAGGAAAGGCC
ATCGTTGAAGATGCCTCTGCCGACAGTGGTCCCAAAGATGGACCCCCACCCACGAGGAGCAT
CGTGGAAAAAGAAGACGTTCCAACCACGTCTTCAAAGCAAGTGGATTGATGTGATAACATGG
TGGAGCACGACACACTTGTCTACTCCAAAAATATCAAAGATACAGTCTCAGAAGACCAAAGG
GCAATTGAGACTTTTCAACAAAGGGTAATATCCGGAAACCTCCTCGGATTCCATTGCCCAGC
TATCTGTCACTTTATTGTGAAGATAGTGGAAAAGGAAGGTGGCTCCTACAAATGCCATCATT
GCGATAAAGGAAAGGCCATCGTTGAAGATGCCTCTGCCGACAGTGGTCCCAAAGATGGACCC
CCACCCACGAGGAGCATCGTGGAAAAAGAAGACGTTCCAACCACGTCTTCAAAGCAAGTGGA
TTGATGTGATATCTCCACTGACGTAAGGGATGACGCACAATCCCACTATCCTTCGCAAGACC
CTTCCTCTATATAAGGAAGTTCATTTCATTTGGAGAGGCACACAATTTGCTTTAGTGATTAA
ACTTTCTTTTACAACAAATTAAAGGTCTATTATCTCCCAACAACATAAGAAAACAATGGCGA
AAAACGTTGCGATTTTCGGCTTATTGTTTTCTCTTCTTGTGTTGGTTCCTTCTCAGATCTTC
GCGACGTCACTCCTCAGCCAAAACGACACCCCCATCTGTCTATCCACTGGCCCCTGGATCTG
CTGCCCAAACTAACTCCATGGTGACCCTGGGATGCCTGGTCAAGGGCTATTTCCCTGAGCCA
GTGACAGTGACCTGGAACTCTGGATCCCTGTCCAGCGGTGTGCACACCTTCCCAGCTGTCCT
GCAGTCTGACCTCTACACTCTGAGCAGCTCAGTGACTGTCCCCTCCAGCACCTGGCCCAGCG
AGACCGTCACCTGCAACGTTGCCCACCCGGCCAGCAGCACCAAGGTGGACAAGAAAATTGTG
CCCAGGGATTGTGGTTGTAAGCCTTGCATATGTACAGTCCCAGAAGTATCATCTGTCTTCAT
CTTCCCCCCAAAGCCCAAGGATGTGCTCACCATTACTCTGACTCCTAAGGTCACGTGTGTTG
TGGTAGACATCAGCAAGGATGATCCCGAGGTCCAGTTCAGCTGGTTTGTAGATGATGTGGAG
GTGCACACAGCTCAGACGCAACCCCGGGAGGAGCAGTTCAACAGCACTTTCCGCTCAGTCAG
TGAACTTCCCATCATGCACCAGGACTGGCTCAATGGCAAGGAGACGTCCAGATTTTGGCGAT
CTATTCAACTGTCGCCAGTTCATTGGTACTGGTAGTCTCCCTGGGGGCAATCAGTTTCTGGA
TGTGCTCTAATGGGTCTCTACAGTGTAGAATATGTATTTAAAGGCCTTAGTCGTGTCGTTTT
TCAAATAATATAATCCTTTTAGGGTTTTAGTTAGTTTAAATTTTCTGTTGCTCCTGTTTAGC
AGGTCGTGCCTTCAGCAAGCACACAAAAACAGAGTGTTTATTTTAAGTTGTTTGTTTAGTGA
TTCAAAAAAAAAATCGTTCAAACATTTGGCAATAAAGTTTCTTAAGATTGAATCCTGTTGCC
GGTCTTGCGATGATTATCATATAATTTCTGTTGAATTACGTTAAGCATGTAATAATTAACAT
GTAATGCATGACGTTATTTATGAGATGGGTTTTTATGATTAGAGTCCCGCAATTATACATTT
AATACGCGATAGAAAACAAAATATAGCGCGCAAACTAGGATAAATTATCGCGCGCGGTGTCA
TCTATGTTACTAGATCTCTAGAGTCTCAAGCTTGGCGCGCCCACGTGACTAGTGGCACTGGC
CGTCGTTTTACAACGTCGTGACTGGGAAAACCCTGGCGTTACCCAACTTAATCGCCTTGCAG
CACATCCCCCTTTCGCCAGCTGGCGTAATAGCGAAGAGGCCCGCACCGATCGCCCTTCCCAA
CAGTTGCGCAGCCTGAATGGCGAATGCTAGAGCAGCTTGAGCTTGGATCAGATTGTCGTTTC
CCGCCTTCAGTTTAAACTATCAGTGTTTGACAGGATATATTGGCGGGTAAACCTAAGAGAAA
AGAGCGTTTA
Construct 8586 from 2X35S prom to NOS term
(SEQ ID NO: 32)
GTCAACATGGTGGAGCACGACACACTTGTCTACTCCAAAAATATCAAAGATACAGTCTCAGA
AGACCAAAGGGCAATTGAGACTTTTCAACAAAGGGTAATATCCGGAAACCTCCTCGGATTCC
ATTGCCCAGCTATCTGTCACTTTATTGTGAAGATAGTGGAAAAGGAAGGTGGCTCCTACAAA
TGCCATCATTGCGATAAAGGAAAGGCCATCGTTGAAGATGCCTCTGCCGACAGTGGTCCCAA
AGATGGACCCCCACCCACGAGGAGCATCGTGGAAAAAGAAGACGTTCCAACCACGTCTTCAA
AGCAAGTGGATTGATGTGATAACATGGTGGAGCACGACACACTTGTCTACTCCAAAAATATC
AAAGATACAGTCTCAGAAGACCAAAGGGCAATTGAGACTTTTCAACAAAGGGTAATATCCGG
AAACCTCCTCGGATTCCATTGCCCAGCTATCTGTCACTTTATTGTGAAGATAGTGGAAAAGG
AAGGTGGCTCCTACAAATGCCATCATTGCGATAAAGGAAAGGCCATCGTTGAAGATGCCTCT
GCCGACAGTGGTCCCAAAGATGGACCCCCACCCACGAGGAGCATCGTGGAAAAAGAAGACGT
TCCAACCACGTCTTCAAAGCAAGTGGATTGATGTGATATCTCCACTGACGTAAGGGATGACG
CACAATCCCACTATCCTTCGCAAGACCCTTCCTCTATATAAGGAAGTTCATTTCATTTGGAG
AGGCACACAATTTGCTTTAGTGATTAAACTTTCTTTTACAACAAATTAAAGGTCTATTATCT
CCCAACAACATAAGAAAACAATGGCGAAAAACGTTGCGATTTTCGGCTTATTGTTTTCTCTT
CTTGTGTTGGTTCCTTCTCAGATCTTCGCGGTGAATCTTACGACGCGAACACAGTTACCACC
CGCATATACAAATAGCTTCACTCGGGGTGTTTATTACCCCGACAAAGTGTTCAGGTCCTCCG
TGCTCCACTCAACACAGGACCTCTTTCTTCCTTTCTTTTCTAACGTGACATGGTTTCATGCC
ATTCATGTATCCGGCACTAACGGTACTAAGAGGTTCGATAATCCTGTGCTCCCTTTCAATGA
CGGCGTTTACTTTGCAAGCACAGAGAAGAGTAACATCATCCGAGGTTGGATCTTTGGCACTA
CCCTCGATTCAAAGACGCAGAGCCTCCTCATTGTGAACAATGCCACTAACGTGGTGATCAAA
GTTTGCGAGTTTCAGTTCTGCAATGACCCTTTCTTGGGGGTGTACTATCATAAGAACAACAA
GTCTTGGATGGAATCTGAATTCCGCGTCTATAGCAGCGCCAACAACTGCACCTTTGAATACG
TGTCCCAGCCCTTCCTTATGGACCTGGAGGGAAAGCAGGGAAACTTTAAGAATCTGAGAGAG
TTCGTGTTTAAAAATATCGACGGCTATTTTAAGATCTATTCTAAGCACACGCCTATTAATCT
CGTGCGCGATCTTCCACAAGGCTTCAGCGCCCTGGAACCACTCGTGGACCTCCCAATTGGTA
TCAACATCACTAGATTTCAGACTCTGCTTGCCCTCCACCGATCCTATCTGACACCCGGAGAC
TCCTCTAGCGGCTGGACTGCCGGCGCTGCCGCTTATTACGTTGGTTATCTTCAGCCACGCAC
GTTCCTGCTGAAGTATAACGAGAATGGTACTATTACCGATGCCGTGGATTGTGCCCTTGACC
CCCTGTCCGAAACTAAGTGCACACTCAAGTCATTCACTGTGGAAAAAGGAATCTACCAGACA
AGCAATTTTCGGGTCCAGCCTACTGAGAGCATTGTGCGCTTTCCTAACATCACAAATCTTTG
CCCCTTCGGAGAGGTTTTCAATGCTACACGGTTTGCCTCCGTGTATGCCTGGAACCGCAAGA
GAATTTCCAATTGCGTGGCCGATTACTCCGTGCTCTACAATAGTGCAAGCTTTAGCACCTTT
AAGTGCTATGGCGTATCCCCTACTAAGCTTAACGACTTGTGTTTCACAAACGTGTATGCCGA
CTCCTTTGTGATACGGGGCGACGAAGTTAGACAGATAGCACCAGGACAGACGGGAAAGATAG
CTGACTACAACTATAAGCTTCCTGATGACTTCACTGGCTGCGTTATCGCGTGGAATTCTAAC
AACCTGGACTCAAAAGTCGGCGGCAACTATAACTATCTCTATCGGCTGTTCCGCAAGAGTAA
CCTTAAGCCCTTTGAGAGAGATATAAGCACTGAAATCTACCAGGCTGGCAGTACGCCCTGTA
ATGGCGTGGAAGGCTTTAATTGTTATTTTCCACTGCAATCCTATGGTTTTCAGCCAACCAAT
GGCGTGGGCTACCAACCATACCGCGTCGTGGTGCTCTCCTTTGAACTGCTCCACGCTCCCGC
GACTGTCTGCGGCCCCAAGAAGTCCACGAACCTTGTGAAGAATAAGTGCGTTAATTTTAATT
TCAACGGCCTCACTGGAACAGGAGTGCTCACTGAGAGTAACAAGAAGTTCCTGCCATTTCAA
CAATTTGGCAGAGACATAGCCGATACTACTGACGCCGTTAGGGACCCCCAGACCCTCGAGAT
TCTCGATATAACGCCCTGCTCCTTCGGTGGAGTTTCCGTGATCACGCCAGGCACCAATACCA
GTAACCAGGTCGCCGTGCTGTATCAGGATGTCAACTGTACTGAGGTGCCCGTAGCCATCCAT
GCGGATCAGCTCACACCAACTTGGAGGGTGTACAGCACCGGCTCCAATGTATTCCAGACTCG
GGCCGGATGCCTTATTGGCGCCGAACACGTGAACAATAGTTACGAATGCGATATTCCAATTG
GCGCCGGAATCTGTGCTAGCTACCAGACTCAGACGAACTCCCCAGGCAGCGCCAGCAGCGTT
GCCAGCCAGTCAATCATCGCTTATACAATGTCACTTGGAGCCGAAAACTCCGTGGCTTACTC
AAACAACAGCATCGCCATCCCCACAAACTTCACCATATCCGTGACAACTGAGATTCTGCCAG
TGTCCATGACTAAGACGTCCGTAGATTGCACTATGTACATATGCGGCGACAGCACAGAATGT
TCTAATCTGCTGCTGCAATATGGAAGCTTCTGCACTCAACTGAACAGAGCGCTCACAGGCAT
CGCCGTGGAGCAGGATAAGAATACCCAGGAGGTGTTCGCCCAAGTTAAGCAGATCTACAAGA
CCCCACCCATAAAGGATTTCGGTGGATTCAATTTTAGTCAGATACTCCCAGACCCATCTAAG
CCATCCAAGAGGAGCTTTATCGAGGATCTTTTGTTTAACAAAGTTACTCTGGCCGACGCCGG
TTTCATCAAGCAGTACGGAGATTGCCTCGGCGACATCGCTGCTCGTGACCTCATCTGTGCGC
AAAAGTTTAACGGTCTGACGGTGCTGCCTCCCCTCCTTACTGATGAAATGATCGCCCAGTAT
ACCAGCGCACTCCTCGCTGGCACCATAACATCCGGTTGGACATTCGGCGCTGGTGCAGCACT
GCAGATACCATTCGCCATGCAAATGGCATATCGTTTCAACGGTATCGGTGTCACACAGAATG
TCCTATATGAGAACCAGAAGCTGATCGCAAATCAGTTCAATAGTGCCATCGGAAAAATCCAG
GATAGCCTTAGCAGCACAGCCTCAGCCCTTGGCAAACTCCAGGATGTCGTGAACCAGAATGC
CCAGGCTCTCAATACCCTCGTGAAGCAGCTCTCATCTAATTTCGGCGCAATTTCCAGTGTCC
TCAACGACATCCTCAGCCGCCTCGACCCCCCCGAGGCCGAAGTGCAGATTGACAGACTGATT
ACAGGTCGACTCCAGAGCCTCCAGACTTACGTGACTCAGCAGCTGATAAGAGCCGCCGAGAT
AAGGGCCAGCGCTAACCTGGCTGCCACAAAGATGTCTGAGTGCGTGCTGGGCCAGTCCAAGA
GAGTAGACTTCTGTGGCAAAGGCTACCATCTGATGAGCTTCCCACAATCCGCACCTCACGGC
GTAGTGTTCCTCCACGTGACATATGTACCGGCTCAGGAGAAGAATTTCACTACCGCTCCTGC
TATATGCCATGATGGAAAGGCTCACTTCCCCCGGGAGGGGGTGTTCGTGTCCAACGGCACCC
ATTGGTTTGTGACTCAGCGGAATTTCTACGAACCCCAGATCATAACCACTGACAACACATTT
GTGTCCGGAAATTGTGACGTGGTCATTGGAATAGTGAACAACACTGTTTATGATCCACTGCA
GCCAGAACTTGACAGCTTTAAGGAGGAGCTCGACAAGTACTTCAAGAATCATACGTCACCAG
ATGTGGACCTCGGAGATATTAGCGGTATCAATGCCAGTGTTGTCAATATTCAGAAGGAAATA
GACCGCCTTAATGAGGTCGCCAAAAATCTGAACGAGAGCCTCATCGATCTTCAGGAGCTGGG
CAAATATGAGCAGTACATCAAGTGGCCTTGGTATATTTGGCTTGGCTTCATCGCCGGCCTGA
TCGCCATAGTAATGGTCACAATTATGCTCTGCTGCATGACCTCTTGCTGCTCCTGTCTGAAA
GGCTGCTGCTCTTGCGGATCCTGCTGCAAATTTGATGAGGATGACAGTGAACCAGTCCTGAA
GGGCGTGAAGCTGCACTATACTTAGAGGCCTTAGTCGTGTCGTTTTTCAAATAATATAATCC
TTTTAGGGTTTTAGTTAGTTTAAATTTTCTGTTGCTCCTGTTTAGCAGGTCGTGCCTTCAGC
AAGCACACAAAAACAGAGTGTTTATTTTAAGTTGTTTGTTTAGTGATTCAAAAAAAAAATCG
TTCAAACATTTGGCAATAAAGTTTCTTAAGATTGAATCCTGTTGCCGGTCTTGCGATGATTA
TCATATAATTTCTGTTGAATTACGTTAAGCATGTAATAATTAACATGTAATGCATGACGTTA
TTTATGAGATGGGTTTTTATGATTAGAGTCCCGCAATTATACATTTAATACGCGATAGAAAA
CAAAATATAGCGCGCAAACTAGGATAAATTATCGCGCGCGGTGTCATCTATGTTACTAGAT
Cloning vector 8500 from left to right T-DNA
(SEQ ID NO: 33)
TGGCAGGATATATTGTGGTGTAAACAAATTGACGCTTAGACAACTTAATAACACATTGCGGA
CGTTTTTAATGTACTGAATTAACGCCGAATCCCGGGCTGGTATATTTATATGTTGTCAAATA
ACTCAAAAACCATAAAAGTTTAAGTTAGCAAGTGTGTACATTTTTACTTGAACAAAAATATT
CACCTACTACTGTTATAAATCATTATTAAACATTAGAGTAAAGAAATATGGATGATAAGAAC
AAGAGTAGTGATATTTTGACAACAATTTTGTTGCAACATTTGAGAAAATTTTGTTGTTCTCT
CTTTTCATTGGTCAAAAACAATAGAGAGAGAAAAAGGAAGAGGGAGAATAAAAACATAATGT
GAGTATGAGAGAGAAAGTTGTACAAAAGTTGTACCAAAATAGTTGTACAAATATCATTGAGG
AATTTGACAAAAGCTACACAAATAAGGGTTAATTGCTGTAAATAAATAAGGATGACGCATTA
GAGAGATGTACCATTAGAGAATTTTTGGCAAGTCATTAAAAAGAAAGAATAAATTATTTTTA
AAATTAAAAGTTGAGTCATTTGATTAAACATGTGATTATTTAATGAATTGATGAAAGAGTTG
GATTAAAGTTGTATTAGTAATTAGAATTTGGTGTCAAATTTAATTTGACATTTGATCTTTTC
CTATATATTGCCCCATAGAGTCAGTTAACTCATTTTTATATTTCATAGATCAAATAAGAGAA
ATAACGGTATATTAATCCCTCCAAAAAAAAAAAACGGTATATTTACTAAAAAATCTAAGCCA
CGTAGGAGGATAACAGGATCCCCGTAGGAGGATAACATCCAATCCAACCAATCACAACAATC
CTGATGAGATAACCCACTTTAAGCCCACGCATCTGTGGCACATCTACATTATCTAAATCACA
CATTCTTCCACACATCTGAGCCACACAAAAACCAATCCACATCTTTATCACCCATTCTATAA
AAAATCACACTTTGTGAGTCTACACTTTGATTCCCTTCAAACACATACAAAGAGAAGAGACT
AATTAATTAATTAATCATCTTGAGAGAAAATGGAACGAGCTATACAAGGAAACGACGCTAGG
GAACAAGCTAACAGTGAACGTTGGGATGGAGGATCAGGAGGTACCACTTCTCCCTTCAAACT
TCCTGACGAAAGTCCGAGTTGGACTGAGTGGCGGCTACATAACGATGAGACGAATTCGAATC
AAGATAATCCCCTTGGTTTCAAGGAAAGCTGGGGTTTCGGGAAAGTTGTATTTAAGAGATAT
CTCAGATACGACAGGACGGAAGCTTCACTGCACAGAGTCCTTGGATCTTGGACGGGAGATTC
GGTTAACTATGCAGCATCTCGATTTTTCGGTTTCGACCAGATCGGATGTACCTATAGTATTC
GGTTTCGAGGAGTTAGTATCACCGTTTCTGGAGGGTCGCGAACTCTTCAGCATCTCTGTGAG
ATGGCAATTCGGTCTAAGCAAGAACTGCTACAGCTTGCCCCAATCGAAGTGGAAAGTAATGT
ATCAAGAGGATGCCCTGAAGGTACTCAAACCTTCGAAAAAGAAAGCGAGTAAGTTAAAATGC
TTCTTCGTCTCCTATTTATAATATGGTTTGTTATTGTTAATTTTGTTCTTGTAGAAGAGCTT
AATTAATCGTTGTTGTTATGAAATACTATTTGTATGAGATGAACTGGTGTAATGTAATTCAT
TTACATAAGTGGAGTCAGAATCAGAATGTTTCCTCCATAACTAACTAGACATGAAGACCTGC
CGCGTACAATTGTCTTATATTTGAACAACTAAAATTGAACATCTTTTGCCACAACTTTATAA
GTGGTTAATATAGCTCAAATATATGGTCAAGTTCAATAGATTAATAATGGAAATATCAGTTA
TCGAAATTCATTAACAATCAACTTAACGTTATTAACTACTAATTTTATATCATCCCCTTTGA
TAAATGATAGTACACCAATTAGGAAGGAGCATGCTCGCCTAGGAGATTGTCGTTTCCCGCCT
TCAGTTTGCAAGCTGCTCTAGCCGTGTAGCCAATACGCAAACCGCCTCTCCCCGCGCGTTGG
GAATTACTAGCGCGTGTCGACAAGCTTGCATGCCGGTCAACATGGTGGAGCACGACACACTT
GTCTACTCCAAAAATATCAAAGATACAGTCTCAGAAGACCAAAGGGCAATTGAGACTTTTCA
ACAAAGGGTAATATCCGGAAACCTCCTCGGATTCCATTGCCCAGCTATCTGTCACTTTATTG
TGAAGATAGTGGAAAAGGAAGGTGGCTCCTACAAATGCCATCATTGCGATAAAGGAAAGGCC
ATCGTTGAAGATGCCTCTGCCGACAGTGGTCCCAAAGATGGACCCCCACCCACGAGGAGCAT
CGTGGAAAAAGAAGACGTTCCAACCACGTCTTCAAAGCAAGTGGATTGATGTGATAACATGG
TGGAGCACGACACACTTGTCTACTCCAAAAATATCAAAGATACAGTCTCAGAAGACCAAAGG
GCAATTGAGACTTTTCAACAAAGGGTAATATCCGGAAACCTCCTCGGATTCCATTGCCCAGC
TATCTGTCACTTTATTGTGAAGATAGTGGAAAAGGAAGGTGGCTCCTACAAATGCCATCATT
GCGATAAAGGAAAGGCCATCGTTGAAGATGCCTCTGCCGACAGTGGTCCCAAAGATGGACCC
CCACCCACGAGGAGCATCGTGGAAAAAGAAGACGTTCCAACCACGTCTTCAAAGCAAGTGGA
TTGATGTGATATCTCCACTGACGTAAGGGATGACGCACAATCCCACTATCCTTCGCAAGACC
CTTCCTCTATATAAGGAAGTTCATTTCATTTGGAGAGGCACTTTTCTAATCAATCATCAAAC
AGAACGCAGAAAATTTCCTAAAAACAAAAAAAAGGCATACAAATGGCGAAAAACGTTGCGAT
TTTCGGCTTATTGTTTTCTCTTCTTGTGTTGGTTCCTTCTCAGATCTTCGCGACGTCACTCC
TCAGCCAAAACGACACCCCCATCTGTCTATCCACTGGCCCCTGGATCTGCTGCCCAAACTAA
CTCCATGGTGACCCTGGGATGCCTGGTCAAGGGCTATTTCCCTGAGCCAGTGACAGTGACCT
GGAACTCTGGATCCCTGTCCAGCGGTGTGCACACCTTCCCAGCTGTCCTGCAGTCTGACCTC
TACACTCTGAGCAGCTCAGTGACTGTCCCCTCCAGCACCTGGCCCAGCGAGACCGTCACCTG
CAACGTTGCCCACCCGGCCAGCAGCACCAAGGTGGACAAGAAAATTGTGCCCAGGGATTGTG
GTTGTAAGCCTTGCATATGTACAGTCCCAGAAGTATCATCTGTCTTCATCTTCCCCCCAAAG
CCCAAGGATGTGCTCACCATTACTCTGACTCCTAAGGTCACGTGTGTTGTGGTAGACATCAG
CAAGGATGATCCCGAGGTCCAGTTCAGCTGGTTTGTAGATGATGTGGAGGTGCACACAGCTC
AGACGCAACCCCGGGAGGAGCAGTTCAACAGCACTTTCCGCTCAGTCAGTGAACTTCCCATC
ATGCACCAGGACTGGCTCAATGGCAAGGAGACGTCCAGATTTTGGCGATCTATTCAACTGTC
GCCAGTTCATTGGTACTGGTAGTCTCCCTGGGGGCAATCAGTTTCTGGATGTGCTCTAATGG
GTCTCTACAGTGTAGAATATGTATTTAAAGGCCTTAGTCGTGTCGTTTTTCAAATAATATAA
TCCTTTTAGGGTTTTAGTTAGTTTAAATTTTCTGTTGCTCCTGTTTAGCAGGTCGTGCCTTC
AGCAAGCACACAAAAACAGAGTGTTTATTTTAAGTTGTTTGTTTAGTGATTCAAAAAAAAAA
TCGTTCAAACATTTGGCAATAAAGTTTCTTAAGATTGAATCCTGTTGCCGGTCTTGCGATGA
TTATCATATAATTTCTGTTGAATTACGTTAAGCATGTAATAATTAACATGTAATGCATGACG
TTATTTATGAGATGGGTTTTTATGATTAGAGTCCCGCAATTATACATTTAATACGCGATAGA
AAACAAAATATAGCGCGCAAACTAGGATAAATTATCGCGCGCGGTGTCATCTATGTTACTAG
ATCTCTAGAGTCTCAAGCTTGGCGCGCCCACGTGACTAGTGGCACTGGCCGTCGTTTTACAA
CGTCGTGACTGGGAAAACCCTGGCGTTACCCAACTTAATCGCCTTGCAGCACATCCCCCTTT
CGCCAGCTGGCGTAATAGCGAAGAGGCCCGCACCGATCGCCCTTCCCAACAGTTGCGCAGCC
TGAATGGCGAATGCTAGAGCAGCTTGAGCTTGGATCAGATTGTCGTTTCCCGCCTTCAGTTT
AAACTATCAGTGTTTGACAGGATATATTGGCGGGTAAACCTAAGAGAAAAGAGCGTTTA
Construct 8589 from 2X35S prom to NOS term
(SEQ ID NO: 34)
GTCAACATGGTGGAGCACGACACACTTGTCTACTCCAAAAATATCAAAGATACAGTCTCAGA
AGACCAAAGGGCAATTGAGACTTTTCAACAAAGGGTAATATCCGGAAACCTCCTCGGATTCC
ATTGCCCAGCTATCTGTCACTTTATTGTGAAGATAGTGGAAAAGGAAGGTGGCTCCTACAAA
TGCCATCATTGCGATAAAGGAAAGGCCATCGTTGAAGATGCCTCTGCCGACAGTGGTCCCAA
AGATGGACCCCCACCCACGAGGAGCATCGTGGAAAAAGAAGACGTTCCAACCACGTCTTCAA
AGCAAGTGGATTGATGTGATAACATGGTGGAGCACGACACACTTGTCTACTCCAAAAATATC
AAAGATACAGTCTCAGAAGACCAAAGGGCAATTGAGACTTTTCAACAAAGGGTAATATCCGG
AAACCTCCTCGGATTCCATTGCCCAGCTATCTGTCACTTTATTGTGAAGATAGTGGAAAAGG
AAGGTGGCTCCTACAAATGCCATCATTGCGATAAAGGAAAGGCCATCGTTGAAGATGCCTCT
GCCGACAGTGGTCCCAAAGATGGACCCCCACCCACGAGGAGCATCGTGGAAAAAGAAGACGT
TCCAACCACGTCTTCAAAGCAAGTGGATTGATGTGATATCTCCACTGACGTAAGGGATGACG
CACAATCCCACTATCCTTCGCAAGACCCTTCCTCTATATAAGGAAGTTCATTTCATTTGGAG
AGGCACTTTTCTAATCAATCATCAAACAGAACGCAGAAAATTTCCTAAAAACAAAAAAAAGG
CATACAAATGGCGAAAAACGTTGCGATTTTCGGCTTATTGTTTTCTCTTCTTGTGTTGGTTC
CTTCTCAGATCTTCGCGGTGAATCTTACGACGCGAACACAGTTACCACCCGCATATACAAAT
AGCTTCACTCGGGGTGTTTATTACCCCGACAAAGTGTTCAGGTCCTCCGTGCTCCACTCAAC
ACAGGACCTCTTTCTTCCTTTCTTTTCTAACGTGACATGGTTTCATGCCATTCATGTATCCG
GCACTAACGGTACTAAGAGGTTCGATAATCCTGTGCTCCCTTTCAATGACGGCGTTTACTTT
GCAAGCACAGAGAAGAGTAACATCATCCGAGGTTGGATCTTTGGCACTACCCTCGATTCAAA
GACGCAGAGCCTCCTCATTGTGAACAATGCCACTAACGTGGTGATCAAAGTTTGCGAGTTTC
AGTTCTGCAATGACCCTTTCTTGGGGGTGTACTATCATAAGAACAACAAGTCTTGGATGGAA
TCTGAATTCCGCGTCTATAGCAGCGCCAACAACTGCACCTTTGAATACGTGTCCCAGCCCTT
CCTTATGGACCTGGAGGGAAAGCAGGGAAACTTTAAGAATCTGAGAGAGTTCGTGTTTAAAA
ATATCGACGGCTATTTTAAGATCTATTCTAAGCACACGCCTATTAATCTCGTGCGCGATCTT
CCACAAGGCTTCAGCGCCCTGGAACCACTCGTGGACCTCCCAATTGGTATCAACATCACTAG
ATTTCAGACTCTGCTTGCCCTCCACCGATCCTATCTGACACCCGGAGACTCCTCTAGCGGCT
GGACTGCCGGCGCTGCCGCTTATTACGTTGGTTATCTTCAGCCACGCACGTTCCTGCTGAAG
TATAACGAGAATGGTACTATTACCGATGCCGTGGATTGTGCCCTTGACCCCCTGTCCGAAAC
TAAGTGCACACTCAAGTCATTCACTGTGGAAAAAGGAATCTACCAGACAAGCAATTTTCGGG
TCCAGCCTACTGAGAGCATTGTGCGCTTTCCTAACATCACAAATCTTTGCCCCTTCGGAGAG
GTTTTCAATGCTACACGGTTTGCCTCCGTGTATGCCTGGAACCGCAAGAGAATTTCCAATTG
CGTGGCCGATTACTCCGTGCTCTACAATAGTGCAAGCTTTAGCACCTTTAAGTGCTATGGCG
TATCCCCTACTAAGCTTAACGACTTGTGTTTCACAAACGTGTATGCCGACTCCTTTGTGATA
CGGGGCGACGAAGTTAGACAGATAGCACCAGGACAGACGGGAAAGATAGCTGACTACAACTA
TAAGCTTCCTGATGACTTCACTGGCTGCGTTATCGCGTGGAATTCTAACAACCTGGACTCAA
AAGTCGGCGGCAACTATAACTATCTCTATCGGCTGTTCCGCAAGAGTAACCTTAAGCCCTTT
GAGAGAGATATAAGCACTGAAATCTACCAGGCTGGCAGTACGCCCTGTAATGGCGTGGAAGG
CTTTAATTGTTATTTTCCACTGCAATCCTATGGTTTTCAGCCAACCAATGGCGTGGGCTACC
AACCATACCGCGTCGTGGTGCTCTCCTTTGAACTGCTCCACGCTCCCGCGACTGTCTGCGGC
CCCAAGAAGTCCACGAACCTTGTGAAGAATAAGTGCGTTAATTTTAATTTCAACGGCCTCAC
TGGAACAGGAGTGCTCACTGAGAGTAACAAGAAGTTCCTGCCATTTCAACAATTTGGCAGAG
ACATAGCCGATACTACTGACGCCGTTAGGGACCCCCAGACCCTCGAGATTCTCGATATAACG
CCCTGCTCCTTCGGTGGAGTTTCCGTGATCACGCCAGGCACCAATACCAGTAACCAGGTCGC
CGTGCTGTATCAGGATGTCAACTGTACTGAGGTGCCCGTAGCCATCCATGCGGATCAGCTCA
CACCAACTTGGAGGGTGTACAGCACCGGCTCCAATGTATTCCAGACTCGGGCCGGATGCCTT
ATTGGCGCCGAACACGTGAACAATAGTTACGAATGCGATATTCCAATTGGCGCCGGAATCTG
TGCTAGCTACCAGACTCAGACGAACTCCCCAGGCAGCGCCAGCAGCGTTGCCAGCCAGTCAA
TCATCGCTTATACAATGTCACTTGGAGCCGAAAACTCCGTGGCTTACTCAAACAACAGCATC
GCCATCCCCACAAACTTCACCATATCCGTGACAACTGAGATTCTGCCAGTGTCCATGACTAA
GACGTCCGTAGATTGCACTATGTACATATGCGGCGACAGCACAGAATGTTCTAATCTGCTGC
TGCAATATGGAAGCTTCTGCACTCAACTGAACAGAGCGCTCACAGGCATCGCCGTGGAGCAG
GATAAGAATACCCAGGAGGTGTTCGCCCAAGTTAAGCAGATCTACAAGACCCCACCCATAAA
GGATTTCGGTGGATTCAATTTTAGTCAGATACTCCCAGACCCATCTAAGCCATCCAAGAGGA
GCTTTATCGAGGATCTTTTGTTTAACAAAGTTACTCTGGCCGACGCCGGTTTCATCAAGCAG
TACGGAGATTGCCTCGGCGACATCGCTGCTCGTGACCTCATCTGTGCGCAAAAGTTTAACGG
TCTGACGGTGCTGCCTCCCCTCCTTACTGATGAAATGATCGCCCAGTATACCAGCGCACTCC
TCGCTGGCACCATAACATCCGGTTGGACATTCGGCGCTGGTGCAGCACTGCAGATACCATTC
GCCATGCAAATGGCATATCGTTTCAACGGTATCGGTGTCACACAGAATGTCCTATATGAGAA
CCAGAAGCTGATCGCAAATCAGTTCAATAGTGCCATCGGAAAAATCCAGGATAGCCTTAGCA
GCACAGCCTCAGCCCTTGGCAAACTCCAGGATGTCGTGAACCAGAATGCCCAGGCTCTCAAT
ACCCTCGTGAAGCAGCTCTCATCTAATTTCGGCGCAATTTCCAGTGTCCTCAACGACATCCT
CAGCCGCCTCGACCCCCCCGAGGCCGAAGTGCAGATTGACAGACTGATTACAGGTCGACTCC
AGAGCCTCCAGACTTACGTGACTCAGCAGCTGATAAGAGCCGCCGAGATAAGGGCCAGCGCT
AACCTGGCTGCCACAAAGATGTCTGAGTGCGTGCTGGGCCAGTCCAAGAGAGTAGACTTCTG
TGGCAAAGGCTACCATCTGATGAGCTTCCCACAATCCGCACCTCACGGCGTAGTGTTCCTCC
ACGTGACATATGTACCGGCTCAGGAGAAGAATTTCACTACCGCTCCTGCTATATGCCATGAT
GGAAAGGCTCACTTCCCCCGGGAGGGGGTGTTCGTGTCCAACGGCACCCATTGGTTTGTGAC
TCAGCGGAATTTCTACGAACCCCAGATCATAACCACTGACAACACATTTGTGTCCGGAAATT
GTGACGTGGTCATTGGAATAGTGAACAACACTGTTTATGATCCACTGCAGCCAGAACTTGAC
AGCTTTAAGGAGGAGCTCGACAAGTACTTCAAGAATCATACGTCACCAGATGTGGACCTCGG
AGATATTAGCGGTATCAATGCCAGTGTTGTCAATATTCAGAAGGAAATAGACCGCCTTAATG
AGGTCGCCAAAAATCTGAACGAGAGCCTCATCGATCTTCAGGAGCTGGGCAAATATGAGCAG
TACATCAAGTGGCCTTGGTATATTTGGCTTGGCTTCATCGCCGGCCTGATCGCCATAGTAAT
GGTCACAATTATGCTCTGCTGCATGACCTCTTGCTGCTCCTGTCTGAAAGGCTGCTGCTCTT
GCGGATCCTGCTGCAAATTTGATGAGGATGACAGTGAACCAGTCCTGAAGGGCGTGAAGCTG
CACTATACTTAGAGGCCTTAGTCGTGTCGTTTTTCAAATAATATAATCCTTTTAGGGTTTTA
GTTAGTTTAAATTTTCTGTTGCTCCTGTTTAGCAGGTCGTGCCTTCAGCAAGCACACAAAAA
CAGAGTGTTTATTTTAAGTTGTTTGTTTAGTGATTCAAAAAAAAAATCGTTCAAACATTTGG
CAATAAAGTTTCTTAAGATTGAATCCTGTTGCCGGTCTTGCGATGATTATCATATAATTTCT
GTTGAATTACGTTAAGCATGTAATAATTAACATGTAATGCATGACGTTATTTATGAGATGGG
TTTTTATGATTAGAGTCCCGCAATTATACATTTAATACGCGATAGAAAACAAAATATAGCGC
GCAAACTAGGATAAATTATCGCGCGCGGTGTCATCTATGTTACTAGAT
Cloning vector 8716 from left to right T-DNA
(SEQ ID NO: 35)
TGGCAGGATATATTGTGGTGTAAACAAATTGACGCTTAGACAACTTAATAACACATTGCGGA
CGTTTTTAATGTACTGAATTAACGCCGAATCCCGGGCTGGTATATTTATATGTTGTCAAATA
ACTCAAAAACCATAAAAGTTTAAGTTAGCAAGTGTGTACATTTTTACTTGAACAAAAATATT
CACCTACTACTGTTATAAATCATTATTAAACATTAGAGTAAAGAAATATGGATGATAAGAAC
AAGAGTAGTGATATTTTGACAACAATTTTGTTGCAACATTTGAGAAAATTTTGTTGTTCTCT
CTTTTCATTGGTCAAAAACAATAGAGAGAGAAAAAGGAAGAGGGAGAATAAAAACATAATGT
GAGTATGAGAGAGAAAGTTGTACAAAAGTTGTACCAAAATAGTTGTACAAATATCATTGAGG
AATTTGACAAAAGCTACACAAATAAGGGTTAATTGCTGTAAATAAATAAGGATGACGCATTA
GAGAGATGTACCATTAGAGAATTTTTGGCAAGTCATTAAAAAGAAAGAATAAATTATTTTTA
AAATTAAAAGTTGAGTCATTTGATTAAACATGTGATTATTTAATGAATTGATGAAAGAGTTG
GATTAAAGTTGTATTAGTAATTAGAATTTGGTGTCAAATTTAATTTGACATTTGATCTTTTC
CTATATATTGCCCCATAGAGTCAGTTAACTCATTTTTATATTTCATAGATCAAATAAGAGAA
ATAACGGTATATTAATCCCTCCAAAAAAAAAAAACGGTATATTTACTAAAAAATCTAAGCCA
CGTAGGAGGATAACAGGATCCCCGTAGGAGGATAACATCCAATCCAACCAATCACAACAATC
CTGATGAGATAACCCACTTTAAGCCCACGCATCTGTGGCACATCTACATTATCTAAATCACA
CATTCTTCCACACATCTGAGCCACACAAAAACCAATCCACATCTTTATCACCCATTCTATAA
AAAATCACACTTTGTGAGTCTACACTTTGATTCCCTTCAAACACATACAAAGAGAAGAGACT
AATTAATTAATTAATCATCTTGAGAGAAAATGGAACGAGCTATACAAGGAAACGACGCTAGG
GAACAAGCTAACAGTGAACGTTGGGATGGAGGATCAGGAGGTACCACTTCTCCCTTCAAACT
TCCTGACGAAAGTCCGAGTTGGACTGAGTGGCGGCTACATAACGATGAGACGAATTCGAATC
AAGATAATCCCCTTGGTTTCAAGGAAAGCTGGGGTTTCGGGAAAGTTGTATTTAAGAGATAT
CTCAGATACGACAGGACGGAAGCTTCACTGCACAGAGTCCTTGGATCTTGGACGGGAGATTC
GGTTAACTATGCAGCATCTCGATTTTTCGGTTTCGACCAGATCGGATGTACCTATAGTATTC
GGTTTCGAGGAGTTAGTATCACCGTTTCTGGAGGGTCGCGAACTCTTCAGCATCTCTGTGAG
ATGGCAATTCGGTCTAAGCAAGAACTGCTACAGCTTGCCCCAATCGAAGTGGAAAGTAATGT
ATCAAGAGGATGCCCTGAAGGTACTCAAACCTTCGAAAAAGAAAGCGAGTAAGTTAAAATGC
TTCTTCGTCTCCTATTTATAATATGGTTTGTTATTGTTAATTTTGTTCTTGTAGAAGAGCTT
AATTAATCGTTGTTGTTATGAAATACTATTTGTATGAGATGAACTGGTGTAATGTAATTCAT
TTACATAAGTGGAGTCAGAATCAGAATGTTTCCTCCATAACTAACTAGACATGAAGACCTGC
CGCGTACAATTGTCTTATATTTGAACAACTAAAATTGAACATCTTTTGCCACAACTTTATAA
GTGGTTAATATAGCTCAAATATATGGTCAAGTTCAATAGATTAATAATGGAAATATCAGTTA
TCGAAATTCATTAACAATCAACTTAACGTTATTAACTACTAATTTTATATCATCCCCTTTGA
TAAATGATAGTACACCAATTAGGAAGGAGCATGCTCGCCTAGGAGATTGTCGTTTCCCGCCT
TCAGTTTGCAAGCTGCTCTAGCCGTGTAGCCAATACGCAAACCGCCTCTCCCCGCGCGTTGG
GAATTACTAGCGCGTGTCGACAAGCTTGCATGCCGGTCAACATGGTGGAGCACGACACACTT
GTCTACTCCAAAAATATCAAAGATACAGTCTCAGAAGACCAAAGGGCAATTGAGACTTTTCA
ACAAAGGGTAATATCCGGAAACCTCCTCGGATTCCATTGCCCAGCTATCTGTCACTTTATTG
TGAAGATAGTGGAAAAGGAAGGTGGCTCCTACAAATGCCATCATTGCGATAAAGGAAAGGCC
ATCGTTGAAGATGCCTCTGCCGACAGTGGTCCCAAAGATGGACCCCCACCCACGAGGAGCAT
CGTGGAAAAAGAAGACGTTCCAACCACGTCTTCAAAGCAAGTGGATTGATGTGATAACATGG
TGGAGCACGACACACTTGTCTACTCCAAAAATATCAAAGATACAGTCTCAGAAGACCAAAGG
GCAATTGAGACTTTTCAACAAAGGGTAATATCCGGAAACCTCCTCGGATTCCATTGCCCAGC
TATCTGTCACTTTATTGTGAAGATAGTGGAAAAGGAAGGTGGCTCCTACAAATGCCATCATT
GCGATAAAGGAAAGGCCATCGTTGAAGATGCCTCTGCCGACAGTGGTCCCAAAGATGGACCC
CCACCCACGAGGAGCATCGTGGAAAAAGAAGACGTTCCAACCACGTCTTCAAAGCAAGTGGA
TTGATGTGATATCTCCACTGACGTAAGGGATGACGCACAATCCCACTATCCTTCGCAAGACC
CTTCCTCTATATAAGGAAGTTCATTTCATTTGGAGAGGCACTCCATTTGAATCTATCAAACC
AAAACACATTGAGCAAAATGGCGAAAAACGTTGCGATTTTCGGCTTATTGTTTTCTCTTCTT
GTGTTGGTTCCTTCTCAGATCTTCGCGACGTCACTCCTCAGCCAAAACGACACCCCCATCTG
TCTATCCACTGGCCCCTGGATCTGCTGCCCAAACTAACTCCATGGTGACCCTGGGATGCCTG
GTCAAGGGCTATTTCCCTGAGCCAGTGACAGTGACCTGGAACTCTGGATCCCTGTCCAGCGG
TGTGCACACCTTCCCAGCTGTCCTGCAGTCTGACCTCTACACTCTGAGCAGCTCAGTGACTG
TCCCCTCCAGCACCTGGCCCAGCGAGACCGTCACCTGCAACGTTGCCCACCCGGCCAGCAGC
ACCAAGGTGGACAAGAAAATTGTGCCCAGGGATTGTGGTTGTAAGCCTTGCATATGTACAGT
CCCAGAAGTATCATCTGTCTTCATCTTCCCCCCAAAGCCCAAGGATGTGCTCACCATTACTC
TGACTCCTAAGGTCACGTGTGTTGTGGTAGACATCAGCAAGGATGATCCCGAGGTCCAGTTC
AGCTGGTTTGTAGATGATGTGGAGGTGCACACAGCTCAGACGCAACCCCGGGAGGAGCAGTT
CAACAGCACTTTCCGCTCAGTCAGTGAACTTCCCATCATGCACCAGGACTGGCTCAATGGCA
AGGAGACGTCCAGATTTTGGCGATCTATTCAACTGTCGCCAGTTCATTGGTACTGGTAGTCT
CCCTGGGGGCAATCAGTTTCTGGATGTGCTCTAATGGGTCTCTACAGTGTAGAATATGTATT
TAAAGGCCTTAGTCGTGTCGTTTTTCAAATAATATAATCCTTTTAGGGTTTTAGTTAGTTTA
AATTTTCTGTTGCTCCTGTTTAGCAGGTCGTGCCTTCAGCAAGCACACAAAAACAGAGTGTT
TATTTTAAGTTGTTTGTTTAGTGATTCAAAAAAAAAATCGTTCAAACATTTGGCAATAAAGT
TTCTTAAGATTGAATCCTGTTGCCGGTCTTGCGATGATTATCATATAATTTCTGTTGAATTA
CGTTAAGCATGTAATAATTAACATGTAATGCATGACGTTATTTATGAGATGGGTTTTTATGA
TTAGAGTCCCGCAATTATACATTTAATACGCGATAGAAAACAAAATATAGCGCGCAAACTAG
GATAAATTATCGCGCGCGGTGTCATCTATGTTACTAGATCTCTAGAGTCTCAAGCTTGGCGC
GCCCACGTGACTAGTGGCACTGGCCGTCGTTTTACAACGTCGTGACTGGGAAAACCCTGGCG
TTACCCAACTTAATCGCCTTGCAGCACATCCCCCTTTCGCCAGCTGGCGTAATAGCGAAGAG
GCCCGCACCGATCGCCCTTCCCAACAGTTGCGCAGCCTGAATGGCGAATGCTAGAGCAGCTT
GAGCTTGGATCAGATTGTCGTTTCCCGCCTTCAGTTTAAACTATCAGTGTTTGACAGGATAT
ATTGGCGGGTAAACCTAAGAGAAAAGAGCGTTTA
Construct 8591 from 2X35S prom to NOS term
(SEQ ID NO: 36)
GTCAACATGGTGGAGCACGACACACTTGTCTACTCCAAAAATATCAAAGATACAGTCTCAGA
AGACCAAAGGGCAATTGAGACTTTTCAACAAAGGGTAATATCCGGAAACCTCCTCGGATTCC
ATTGCCCAGCTATCTGTCACTTTATTGTGAAGATAGTGGAAAAGGAAGGTGGCTCCTACAAA
TGCCATCATTGCGATAAAGGAAAGGCCATCGTTGAAGATGCCTCTGCCGACAGTGGTCCCAA
AGATGGACCCCCACCCACGAGGAGCATCGTGGAAAAAGAAGACGTTCCAACCACGTCTTCAA
AGCAAGTGGATTGATGTGATAACATGGTGGAGCACGACACACTTGTCTACTCCAAAAATATC
AAAGATACAGTCTCAGAAGACCAAAGGGCAATTGAGACTTTTCAACAAAGGGTAATATCCGG
AAACCTCCTCGGATTCCATTGCCCAGCTATCTGTCACTTTATTGTGAAGATAGTGGAAAAGG
AAGGTGGCTCCTACAAATGCCATCATTGCGATAAAGGAAAGGCCATCGTTGAAGATGCCTCT
GCCGACAGTGGTCCCAAAGATGGACCCCCACCCACGAGGAGCATCGTGGAAAAAGAAGACGT
TCCAACCACGTCTTCAAAGCAAGTGGATTGATGTGATATCTCCACTGACGTAAGGGATGACG
CACAATCCCACTATCCTTCGCAAGACCCTTCCTCTATATAAGGAAGTTCATTTCATTTGGAG
AGGCACTCCATTTGAATCTATCAAACCAAAACACATTGAGCAAAATGGCGAAAAACGTTGCG
ATTTTCGGCTTATTGTTTTCTCTTCTTGTGTTGGTTCCTTCTCAGATCTTCGCGGTGAATCT
TACGACGCGAACACAGTTACCACCCGCATATACAAATAGCTTCACTCGGGGTGTTTATTACC
CCGACAAAGTGTTCAGGTCCTCCGTGCTCCACTCAACACAGGACCTCTTTCTTCCTTTCTTT
TCTAACGTGACATGGTTTCATGCCATTCATGTATCCGGCACTAACGGTACTAAGAGGTTCGA
TAATCCTGTGCTCCCTTTCAATGACGGCGTTTACTTTGCAAGCACAGAGAAGAGTAACATCA
TCCGAGGTTGGATCTTTGGCACTACCCTCGATTCAAAGACGCAGAGCCTCCTCATTGTGAAC
AATGCCACTAACGTGGTGATCAAAGTTTGCGAGTTTCAGTTCTGCAATGACCCTTTCTTGGG
GGTGTACTATCATAAGAACAACAAGTCTTGGATGGAATCTGAATTCCGCGTCTATAGCAGCG
CCAACAACTGCACCTTTGAATACGTGTCCCAGCCCTTCCTTATGGACCTGGAGGGAAAGCAG
GGAAACTTTAAGAATCTGAGAGAGTTCGTGTTTAAAAATATCGACGGCTATTTTAAGATCTA
TTCTAAGCACACGCCTATTAATCTCGTGCGCGATCTTCCACAAGGCTTCAGCGCCCTGGAAC
CACTCGTGGACCTCCCAATTGGTATCAACATCACTAGATTTCAGACTCTGCTTGCCCTCCAC
CGATCCTATCTGACACCCGGAGACTCCTCTAGCGGCTGGACTGCCGGCGCTGCCGCTTATTA
CGTTGGTTATCTTCAGCCACGCACGTTCCTGCTGAAGTATAACGAGAATGGTACTATTACCG
ATGCCGTGGATTGTGCCCTTGACCCCCTGTCCGAAACTAAGTGCACACTCAAGTCATTCACT
GTGGAAAAAGGAATCTACCAGACAAGCAATTTTCGGGTCCAGCCTACTGAGAGCATTGTGCG
CTTTCCTAACATCACAAATCTTTGCCCCTTCGGAGAGGTTTTCAATGCTACACGGTTTGCCT
CCGTGTATGCCTGGAACCGCAAGAGAATTTCCAATTGCGTGGCCGATTACTCCGTGCTCTAC
AATAGTGCAAGCTTTAGCACCTTTAAGTGCTATGGCGTATCCCCTACTAAGCTTAACGACTT
GTGTTTCACAAACGTGTATGCCGACTCCTTTGTGATACGGGGCGACGAAGTTAGACAGATAG
CACCAGGACAGACGGGAAAGATAGCTGACTACAACTATAAGCTTCCTGATGACTTCACTGGC
TGCGTTATCGCGTGGAATTCTAACAACCTGGACTCAAAAGTCGGCGGCAACTATAACTATCT
CTATCGGCTGTTCCGCAAGAGTAACCTTAAGCCCTTTGAGAGAGATATAAGCACTGAAATCT
ACCAGGCTGGCAGTACGCCCTGTAATGGCGTGGAAGGCTTTAATTGTTATTTTCCACTGCAA
TCCTATGGTTTTCAGCCAACCAATGGCGTGGGCTACCAACCATACCGCGTCGTGGTGCTCTC
CTTTGAACTGCTCCACGCTCCCGCGACTGTCTGCGGCCCCAAGAAGTCCACGAACCTTGTGA
AGAATAAGTGCGTTAATTTTAATTTCAACGGCCTCACTGGAACAGGAGTGCTCACTGAGAGT
AACAAGAAGTTCCTGCCATTTCAACAATTTGGCAGAGACATAGCCGATACTACTGACGCCGT
TAGGGACCCCCAGACCCTCGAGATTCTCGATATAACGCCCTGCTCCTTCGGTGGAGTTTCCG
TGATCACGCCAGGCACCAATACCAGTAACCAGGTCGCCGTGCTGTATCAGGATGTCAACTGT
ACTGAGGTGCCCGTAGCCATCCATGCGGATCAGCTCACACCAACTTGGAGGGTGTACAGCAC
CGGCTCCAATGTATTCCAGACTCGGGCCGGATGCCTTATTGGCGCCGAACACGTGAACAATA
GTTACGAATGCGATATTCCAATTGGCGCCGGAATCTGTGCTAGCTACCAGACTCAGACGAAC
TCCCCAGGCAGCGCCAGCAGCGTTGCCAGCCAGTCAATCATCGCTTATACAATGTCACTTGG
AGCCGAAAACTCCGTGGCTTACTCAAACAACAGCATCGCCATCCCCACAAACTTCACCATAT
CCGTGACAACTGAGATTCTGCCAGTGTCCATGACTAAGACGTCCGTAGATTGCACTATGTAC
ATATGCGGCGACAGCACAGAATGTTCTAATCTGCTGCTGCAATATGGAAGCTTCTGCACTCA
ACTGAACAGAGCGCTCACAGGCATCGCCGTGGAGCAGGATAAGAATACCCAGGAGGTGTTCG
CCCAAGTTAAGCAGATCTACAAGACCCCACCCATAAAGGATTTCGGTGGATTCAATTTTAGT
CAGATACTCCCAGACCCATCTAAGCCATCCAAGAGGAGCTTTATCGAGGATCTTTTGTTTAA
CAAAGTTACTCTGGCCGACGCCGGTTTCATCAAGCAGTACGGAGATTGCCTCGGCGACATCG
CTGCTCGTGACCTCATCTGTGCGCAAAAGTTTAACGGTCTGACGGTGCTGCCTCCCCTCCTT
ACTGATGAAATGATCGCCCAGTATACCAGCGCACTCCTCGCTGGCACCATAACATCCGGTTG
GACATTCGGCGCTGGTGCAGCACTGCAGATACCATTCGCCATGCAAATGGCATATCGTTTCA
ACGGTATCGGTGTCACACAGAATGTCCTATATGAGAACCAGAAGCTGATCGCAAATCAGTTC
AATAGTGCCATCGGAAAAATCCAGGATAGCCTTAGCAGCACAGCCTCAGCCCTTGGCAAACT
CCAGGATGTCGTGAACCAGAATGCCCAGGCTCTCAATACCCTCGTGAAGCAGCTCTCATCTA
ATTTCGGCGCAATTTCCAGTGTCCTCAACGACATCCTCAGCCGCCTCGACCCCCCCGAGGCC
GAAGTGCAGATTGACAGACTGATTACAGGTCGACTCCAGAGCCTCCAGACTTACGTGACTCA
GCAGCTGATAAGAGCCGCCGAGATAAGGGCCAGCGCTAACCTGGCTGCCACAAAGATGTCTG
AGTGCGTGCTGGGCCAGTCCAAGAGAGTAGACTTCTGTGGCAAAGGCTACCATCTGATGAGC
TTCCCACAATCCGCACCTCACGGCGTAGTGTTCCTCCACGTGACATATGTACCGGCTCAGGA
GAAGAATTTCACTACCGCTCCTGCTATATGCCATGATGGAAAGGCTCACTTCCCCCGGGAGG
GGGTGTTCGTGTCCAACGGCACCCATTGGTTTGTGACTCAGCGGAATTTCTACGAACCCCAG
ATCATAACCACTGACAACACATTTGTGTCCGGAAATTGTGACGTGGTCATTGGAATAGTGAA
CAACACTGTTTATGATCCACTGCAGCCAGAACTTGACAGCTTTAAGGAGGAGCTCGACAAGT
ACTTCAAGAATCATACGTCACCAGATGTGGACCTCGGAGATATTAGCGGTATCAATGCCAGT
GTTGTCAATATTCAGAAGGAAATAGACCGCCTTAATGAGGTCGCCAAAAATCTGAACGAGAG
CCTCATCGATCTTCAGGAGCTGGGCAAATATGAGCAGTACATCAAGTGGCCTTGGTATATTT
GGCTTGGCTTCATCGCCGGCCTGATCGCCATAGTAATGGTCACAATTATGCTCTGCTGCATG
ACCTCTTGCTGCTCCTGTCTGAAAGGCTGCTGCTCTTGCGGATCCTGCTGCAAATTTGATGA
GGATGACAGTGAACCAGTCCTGAAGGGCGTGAAGCTGCACTATACTTAGAGGCCTTAGTCGT
GTCGTTTTTCAAATAATATAATCCTTTTAGGGTTTTAGTTAGTTTAAATTTTCTGTTGCTCC
TGTTTAGCAGGTCGTGCCTTCAGCAAGCACACAAAAACAGAGTGTTTATTTTAAGTTGTTTG
TTTAGTGATTCAAAAAAAAAATCGTTCAAACATTTGGCAATAAAGTTTCTTAAGATTGAATC
CTGTTGCCGGTCTTGCGATGATTATCATATAATTTCTGTTGAATTACGTTAAGCATGTAATA
ATTAACATGTAATGCATGACGTTATTTATGAGATGGGTTTTTATGATTAGAGTCCCGCAATT
ATACATTTAATACGCGATAGAAAACAAAATATAGCGCGCAAACTAGGATAAATTATCGCGCG
CGGTGTCATCTATGTTACTAGAT
C-Terminal Region of Modified SARS-CoV-2 S protein with H5i Hemagglutinin CT,
Variation 2
(SEQ ID NO: 37)
WYIWLGFIAGLIAIVMVTIMAGLSLWMCSNGSLQCRICI
C-Terminal Region of Modified SARS-CoV-2 S protein with H5i Hemagglutinin CT,
Variation 3
(SEQ ID NO: 38)
WYIWLGFIAGLIAIVMVTIMLCCMCSNGSLQCRICI
C-Terminal Region of Modified SARS-CoV-2 S protein with H5i Hemagglutinin CT,
Variation 4
(SEQ ID NO: 39)
WYIWLGFIAGLIAIVMVTIMLCCSNGSLQCRICI
3′UTR AvB (Arracacha virus B)
(SEQ ID NO: 40)
TAGTCGTGTCGTTTTTCAAATAATATAATCCTTTTAGGGTTTTAGTTAGTTTAAATTTTCTG
TTGCTCCTGTTTAGCAGGTCGTGCCTTCAGCAAGCACACAAAAACAGAGTGTTTATTTTAAG
TTGTTTGTTTAGTGATTCAAAAAAAAA
3′UTR trBNYVV (Beet necrotic yellow vein virus)
(SEQ ID NO: 41)
CCTATCTTGATGAAGGTTGTTGTGGTTTTCTCATTACTGTTTTATTATTGTTTGAGTTGCTT
ATGTCGGTTCTTGATTATGTGGTGCATAATTATTGAACTAATTGTTTGTTGGGTTGTAATGT
ACTGACTGGGTGTGAATTGTACCAGTCGTTAAAGGGTTTACTATCAGTATATTGATAT
3′ UTR SBMV (Southern bean mosaic virus)
(SEQ ID NO: 42)
TGAGGAGTTGTATAATAATACCTGCACCCTTCTCTTTGGCAGGGAGGGTGTTTCGTTTTCAC
AATGCCACGCGCTTGAGGGAGAATGCACGTTAATCATCCCTCCGCTAGTGATGGAGCGTAAT
CCAAAAGT
3′ UTR TuRSV (Turnip ringspot virus)
(SEQ ID NO: 43)
TGATTTATAATAGCCATAGATTAAGTTTAAATGTATTACGTTTGTATTTTATTCTCTTTTTT
AAGTTTCCTATGTTGTTTTAAATTAAATATCTGTATAATTAGTAGATGTAAATCTGCTTTGT
GCGTTTGACAGTCTGTGGAAACGCACTGGTTCATGAGATAGGACCACCTAGGAGGTAGGACT
CTGGGTTTTAATTATCTCATTTCTTAGTTATACCGTATTATATATATGATTTAGTAGTAATT
GTTTTCTCTTGATATGTATTATTACTTTTTTATT
3′ UTR CPMV (Cowpea Mosaic virus)
(SEQ ID NO: 44)
ATTTTCTTTAGTTTGAATTTACTGTTATTCGGTGTGCATTTCTATGTTTGGTGAGCGGTTTT
CTGTGCTCAGAGTGTGTTTATTTTATGTAATTTAATTTCTTTGTGAGCTCCTGTTTAGCAGG
TCGTCCCTTCAGCAAGGACACAAAAAGATTTTAATTTTATT
3′ UTR BBTMV (Broad bean true mosaic virus)
(SEQ ID NO: 45)
TAGTTTTCTTCCGCTTTTCTTTTGTAGTGTGTGGTTTTCTTTGTTTCTTCTTTTCTTTTCTC
TTTCCTTTTCTCTTACTCCTGCCTGGCAGGTCGTGCCTTCAGTAAGCACAACAAAAATATGC
ATTTATTAGAGTATTTCTTTCTTCTTTAGCATAAAGGTATTGAAGACCTATAAACTTCGTCC
GGGTTGGGGAAAGTACCAGCTTAGCATATCTTTAGAAAACTATATAGAGCTCTTTACCTTGA
GTTGTTTCCTAAAGTTTATGCAAAAAA
3′ UTR trOUMV (Ourmia melon virus)
(SEQ ID NO: 46)
CTCACGTCTGGGGTGAGCCCTAGCCAAATAGGAAAACGATAAGCGCTTTGCATGCAAAATGA
GTTGGGCCACAAGTGCCACTCGCAGCGAAGGCGGTCTGAGGTTTCCCCCTGGCGGTTACTTC
CATATCTTTGGGAGATAACTGGG
(PDI) Modified SARS-CoV-2 S protein GSAS + PP with H5i Hemagglutinin CT AA
(SEQ ID NO: 47)
MAKNVAIFGLLFSLLVLVPSQIFAVNLTTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANNCTFEYVSQPFLM
DLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSETKC
TLKSFTVEKGIYQTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQAGSTPCNGVEGFN
CYFPLQSYGFQPTNGVGYQPYRVVVLSFELLHAPATVCGPKKSTNLVKNKCVNFNFNGLTGT
GVLTESNKKFLPFQQFGRDIADTTDAVRDPQTLEILDITPCSFGGVSVITPGTNTSNQVAVL
YQDVNCTEVPVAIHADQLTPTWRVYSTGSNVFQTRAGCLIGAEHVNNSYECDIPIGAGICAS
YQTQTNSPGSASSVASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQVKQIYKTPPIKDF
GGFNFSQILPDPSKPSKRSFIEDLLFNKVTLADAGFIKQYGDCLGDIAARDLICAQKFNGLT
VLPPLLTDEMIAQYTSALLAGTITSGWTFGAGAALQIPFAMQMAYRFNGIGVTQNVLYENQK
LIANQFNSAIGKIQDSLSSTASALGKLQDVVNQNAQALNTLVKQLSSNFGAISSVLNDILSR
LDPPEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKYFKNHTSPDVDLGDI
SGINASVVNIQKEIDRLNEVAKNLNESLIDLQELGKYEQYIKWPWYIWLGFIAGLIAIVMVT
IMLSLWMCSNGSLQCRICI
(PDI) Modified SARS-CoV-2 S protein GSAS + 4P with H5i Hemagglutinin CT AA
(SEQ ID NO: 48)
MAKNVAIFGLLFSLLVLVPSQIFAVNLTTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANNCTFEYVSQPFLM
DLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSETKC
TLKSFTVEKGIYQTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQAGSTPCNGVEGFN
CYFPLQSYGFQPTNGVGYQPYRVVVLSFELLHAPATVCGPKKSTNLVKNKCVNFNFNGLTGT
GVLTESNKKFLPFQQFGRDIADTTDAVRDPQTLEILDITPCSFGGVSVITPGTNTSNQVAVL
YQDVNCTEVPVAIHADQLTPTWRVYSTGSNVFQTRAGCLIGAEHVNNSYECDIPIGAGICAS
YQTQTNSPGSASSVASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQVKQIYKTPPIKDF
GGFNFSQILPDPSKPSKRSPIEDLLFNKVTLADAGFIKQYGDCLGDIAARDLICAQKFNGLT
VLPPLLTDEMIAQYTSALLAGTITSGWTFGAGAALQIPFAMQMAYRFNGIGVTQNVLYENQK
LIANQFNSAIGKIQDSLSSTPSALGKLQDVVNQNAQALNTLVKQLSSNFGAISSVLNDILSR
LDPPEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKYFKNHTSPDVDLGDI
SGINASVVNIQKEIDRLNEVAKNLNESLIDLQELGKYEQYIKWPWYIWLGFIAGLIAIVMVT
IMLSLWMCSNGSLQCRICI
(PDI) Modified SARS-CoV-2 S protein GSAS + 6P with H5i Hemagglutinin CT AA
(SEQ ID NO: 49)
MAKNVAIFGLLFSLLVLVPSQIFAVNLTTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANNCTFEYVSQPFLM
DLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSETKC
TLKSFTVEKGIYQTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQAGSTPCNGVEGFN
CYFPLQSYGFQPTNGVGYQPYRVVVLSFELLHAPATVCGPKKSTNLVKNKCVNFNFNGLTGT
GVLTESNKKFLPFQQFGRDIADTTDAVRDPQTLEILDITPCSFGGVSVITPGTNTSNQVAVL
YQDVNCTEVPVAIHADQLTPTWRVYSTGSNVFQTRAGCLIGAEHVNNSYECDIPIGAGICAS
YQTQTNSPGSASSVASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQVKQIYKTPPIKDF
GGFNFSQILPDPSKPSKRSPIEDLLFNKVTLADAGFIKQYGDCLGDIAARDLICAQKFNGLT
VLPPLLTDEMIAQYTSALLAGTITSGWTFGAGPALQIPFPMQMAYRENGIGVTQNVLYENQK
LIANQFNSAIGKIQDSLSSTPSALGKLQDVVNQNAQALNTLVKQLSSNFGAISSVLNDILSR
LDPPEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKYFKNHTSPDVDLGDI
SGINASVVNIQKEIDRLNEVAKNLNESLIDLQELGKYEQYIKWPWYIWLGFIAGLIAIVMVT
IMLSLWMCSNGSLQCRICI
(PDI) Modified SARS-CoV-2 S protein GSAS + PP + 923 with H5i Hemagglutinin CT AA
(SEQ ID NO: 50)
MAKNVAIFGLLFSLLVLVPSQIFAVNLTTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANNCTFEYVSQPFLM
DLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSETKC
TLKSFTVEKGIYQTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQAGSTPCNGVEGFN
CYFPLQSYGFQPTNGVGYQPYRVVVLSFELLHAPATVCGPKKSTNLVKNKCVNFNFNGLTGT
GVLTESNKKFLPFQQFGRDIADTTDAVRDPQTLEILDITPCSFGGVSVITPGTNTSNQVAVL
YQDVNCTEVPVAIHADQLTPTWRVYSTGSNVFQTRAGCLIGAEHVNNSYECDIPIGAGICAS
YQTQTNSPGSASSVASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQVKQIYKTPPIKDF
GGFNFSQILPDPSKPSKRSFIEDLLFNKVTLADAGFIKQYGDCLGDIAARDLICAQKFNGLT
VLPPLLTDEMIAQYTSALLAGTITSGWTFGAGAALQIPFAMQMAYRFNGIGVTQNVLYENQK
LIANQFNSAIGKIQDSFSSTASALGKLQDVVNQNAQALNTLVKQLSSNFGAISSVLNDILSR
LDPPEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKYFKNHTSPDVDLGDI
SGINASVVNIQKEIDRLNEVAKNLNESLIDLQELGKYEQYIKWPWYIWLGFIAGLIAIVMVT
IMLSLWMCSNGSLQCRICI
(PDI) Modified SARS-CoV-2 S protein GSAS + 4P + 923 with H5i Hemagglutinin CT AA
(SEQ ID NO: 51)
MAKNVAIFGLLFSLLVLVPSQIFAVNLTTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANNCTFEYVSQPFLM
DLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSETKC
TLKSFTVEKGIYQTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQAGSTPCNGVEGFN
CYFPLQSYGFQPTNGVGYQPYRVVVLSFELLHAPATVCGPKKSTNLVKNKCVNFNFNGLTGT
GVLTESNKKFLPFQQFGRDIADTTDAVRDPQTLEILDITPCSFGGVSVITPGTNTSNQVAVL
YQDVNCTEVPVAIHADQLTPTWRVYSTGSNVFQTRAGCLIGAEHVNNSYECDIPIGAGICAS
YQTQTNSPGSASSVASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQVKQIYKTPPIKDF
GGFNFSQILPDPSKPSKRSPIEDLLFNKVTLADAGFIKQYGDCLGDIAARDLICAQKFNGLT
VLPPLLTDEMIAQYTSALLAGTITSGWTFGAGAALQIPFAMQMAYRFNGIGVTQNVLYENQK
LIANQFNSAIGKIQDSFSSTPSALGKLQDVVNQNAQALNTLVKQLSSNFGAISSVLNDILSR
LDPPEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKYFKNHTSPDVDLGDI
SGINASVVNIQKEIDRLNEVAKNLNESLIDLQELGKYEQYIKWPWYIWLGFIAGLIAIVMVT
IMLSLWMCSNGSLQCRICI
(PDI) Modified SARS-CoV-2 S protein GSAS + 6P + 923 with H5i Hemagglutinin CT AA
(SEQ ID NO: 52)
MAKNVAIFGLLFSLLVLVPSQIFAVNLTTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANNCTFEYVSQPFLM
DLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSETKC
TLKSFTVEKGIYQTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQAGSTPCNGVEGFN
CYFPLQSYGFQPTNGVGYQPYRVVVLSFELLHAPATVCGPKKSTNLVKNKCVNFNFNGLTGT
GVLTESNKKFLPFQQFGRDIADTTDAVRDPQTLEILDITPCSFGGVSVITPGTNTSNQVAVL
YQDVNCTEVPVAIHADQLTPTWRVYSTGSNVFQTRAGCLIGAEHVNNSYECDIPIGAGICAS
YQTQTNSPGSASSVASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQVKQIYKTPPIKDF
GGFNFSQILPDPSKPSKRSPIEDLLFNKVTLADAGFIKQYGDCLGDIAARDLICAQKFNGLT
VLPPLLTDEMIAQYTSALLAGTITSGWTFGAGPALQIPFPMQMAYRFNGIGVTQNVLYENQK
LIANQFNSAIGKIQDSFSSTPSALGKLQDVVNQNAQALNTLVKQLSSNFGAISSVLNDILSR
LDPPEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKYFKNHTSPDVDLGDI
SGINASVVNIQKEIDRLNEVAKNLNESLIDLQELGKYEQYIKWPWYIWLGFIAGLIAIVMVT
IMLSLWMCSNGSLQCRICI
(PDI) Modified SARS-CoV-2 S protein GSAS + PP with H1 Cal Hemagglutinin CT AA
(SEQ ID NO: 53)
MAKNVAIFGLLFSLLVLVPSQIFAVNLTTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANNCTFEYVSQPFLM
DLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSETKC
TLKSFTVEKGIYQTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQAGSTPCNGVEGFN
CYFPLQSYGFQPTNGVGYQPYRVVVLSFELLHAPATVCGPKKSTNLVKNKCVNFNFNGLTGT
GVLTESNKKFLPFQQFGRDIADTTDAVRDPQTLEILDITPCSFGGVSVITPGTNTSNQVAVL
YQDVNCTEVPVAIHADQLTPTWRVYSTGSNVFQTRAGCLIGAEHVNNSYECDIPIGAGICAS
YQTQTNSPGSASSVASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQVKQIYKTPPIKDF
GGFNFSQILPDPSKPSKRSFIEDLLFNKVTLADAGFIKQYGDCLGDIAARDLICAQKFNGLT
VLPPLLTDEMIAQYTSALLAGTITSGWTFGAGAALQIPFAMQMAYRFNGIGVTQNVLYENQK
LIANQFNSAIGKIQDSLSSTASALGKLQDVVNQNAQALNTLVKQLSSNFGAISSVLNDILSR
LDPPEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKYFKNHTSPDVDLGDI
SGINASVVNIQKEIDRLNEVAKNLNESLIDLQELGKYEQYIKWPWYIWLGFIAGLIAIVMVT
IMLSFWMCSNGSLQCRICI
(PDI) Modified SARS-CoV-2 S protein GSAS + PP with H3 Minn Hemagglutinin CT AA
(SEQ ID NO: 54)
MAKNVAIFGLLFSLLVLVPSQIFAVNLTTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANNCTFEYVSQPFLM
DLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSETKC
TLKSFTVEKGIYQTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQAGSTPCNGVEGFN
CYFPLQSYGFQPTNGVGYQPYRVVVLSFELLHAPATVCGPKKSTNLVKNKCVNFNFNGLTGT
GVLTESNKKFLPFQQFGRDIADTTDAVRDPQTLEILDITPCSFGGVSVITPGTNTSNQVAVL
YQDVNCTEVPVAIHADQLTPTWRVYSTGSNVFQTRAGCLIGAEHVNNSYECDIPIGAGICAS
YQTQTNSPGSASSVASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQVKQIYKTPPIKDF
GGFNFSQILPDPSKPSKRSFIEDLLFNKVTLADAGFIKQYGDCLGDIAARDLICAQKFNGLT
VLPPLLTDEMIAQYTSALLAGTITSGWTFGAGAALQIPFAMQMAYRFNGIGVTQNVLYENQK
LIANQFNSAIGKIQDSLSSTASALGKLQDVVNQNAQALNTLVKQLSSNFGAISSVLNDILSR
LDPPEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKYFKNHTSPDVDLGDI
SGINASVVNIQKEIDRLNEVAKNLNESLIDLQELGKYEQYIKWPWYIWLGFIAGLIAIVMVT
IMLMWACQKGNIRCNICI
(PDI) Modified SARS-CoV-2 S protein GSAS + PP with H6 HK Hemagglutinin CT AA
(SEQ ID NO: 55)
MAKNVAIFGLLFSLLVLVPSQIFAVNLTTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANNCTFEYVSQPFLM
DLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSETKC
TLKSFTVEKGIYQTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQAGSTPCNGVEGFN
CYFPLQSYGFQPTNGVGYQPYRVVVLSFELLHAPATVCGPKKSTNLVKNKCVNFNFNGLTGT
GVLTESNKKFLPFQQFGRDIADTTDAVRDPQTLEILDITPCSFGGVSVITPGTNTSNQVAVL
YQDVNCTEVPVAIHADQLTPTWRVYSTGSNVFQTRAGCLIGAEHVNNSYECDIPIGAGICAS
YQTQTNSPGSASSVASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQVKQIYKTPPIKDF
GGFNFSQILPDPSKPSKRSFIEDLLFNKVTLADAGFIKQYGDCLGDIAARDLICAQKFNGLT
VLPPLLTDEMIAQYTSALLAGTITSGWTFGAGAALQIPFAMQMAYRFNGIGVTQNVLYENQK
LIANQFNSAIGKIQDSLSSTASALGKLQDVVNQNAQALNTLVKQLSSNFGAISSVLNDILSR
LDPPEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKYFKNHTSPDVDLGDI
SGINASVVNIQKEIDRLNEVAKNLNESLIDLQELGKYEQYIKWPWYIWLGFIAGLIAIVMVT
IMLGLWMCSNGSMQCRICI
(PDI) Modified SARS-CoV-2 S protein GSAS + PP with H7 Guangdong Hemagglutinin CT
AA
(SEQ ID NO: 56)
MAKNVAIFGLLFSLLVLVPSQIFAVNLTTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANNCTFEYVSQPFLM
DLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSETKC
TLKSFTVEKGIYQTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQAGSTPCNGVEGFN
CYFPLQSYGFQPTNGVGYQPYRVVVLSFELLHAPATVCGPKKSTNLVKNKCVNFNFNGLTGT
GVLTESNKKFLPFQQFGRDIADTTDAVRDPQTLEILDITPCSFGGVSVITPGTNTSNQVAVL
YQDVNCTEVPVAIHADQLTPTWRVYSTGSNVFQTRAGCLIGAEHVNNSYECDIPIGAGICAS
YQTQTNSPGSASSVASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQVKQIYKTPPIKDF
GGFNFSQILPDPSKPSKRSFIEDLLFNKVTLADAGFIKQYGDCLGDIAARDLICAQKFNGLT
VLPPLLTDEMIAQYTSALLAGTITSGWTFGAGAALQIPFAMQMAYRFNGIGVTQNVLYENQK
LIANQFNSAIGKIQDSLSSTASALGKLQDVVNQNAQALNTLVKQLSSNFGAISSVLNDILSR
LDPPEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKYFKNHTSPDVDLGDI
SGINASVVNIQKEIDRLNEVAKNLNESLIDLQELGKYEQYIKWPWYIWLGFIAGLIAIVMVT
IMLVFICVKNGNMRCTICI
(PDI) Modified SARS-CoV-2 S protein GSAS + PP with H9 HK Hemagglutinin CT AA
(SEQ ID NO: 57)
MAKNVAIFGLLFSLLVLVPSQIFAVNLTTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANNCTFEYVSQPFLM
DLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSETKC
TLKSFTVEKGIYQTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQAGSTPCNGVEGFN
CYFPLQSYGFQPTNGVGYQPYRVVVLSFELLHAPATVCGPKKSTNLVKNKCVNFNFNGLTGT
GVLTESNKKFLPFQQFGRDIADTTDAVRDPOTLEILDITPCSFGGVSVITPGTNTSNQVAVL
YQDVNCTEVPVAIHADQLTPTWRVYSTGSNVFQTRAGCLIGAEHVNNSYECDIPIGAGICAS
YQTQTNSPGSASSVASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQVKQIYKTPPIKDF
GGFNFSQILPDPSKPSKRSFIEDLLFNKVTLADAGFIKQYGDCLGDIAARDLICAQKFNGLT
VLPPLLTDEMIAQYTSALLAGTITSGWTFGAGAALQIPFAMQMAYRFNGIGVTQNVLYENQK
LIANQFNSAIGKIQDSLSSTASALGKLQDVVNQNAQALNTLVKQLSSNFGAISSVLNDILSR
LDPPEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKYFKNHTSPDVDLGDI
SGINASVVNIQKEIDRLNEVAKNLNESLIDLQELGKYEQYIKWPWYIWLGFIAGLIAIVMVT
IMLLFWAMSNGSCRCNICI
(PDI) Modified SARS-CoV-2 S protein GSAS + PP with B/Wash Hemagglutinin CT AA
(SEQ ID NO: 58)
MAKNVAIFGLLFSLLVLVPSQIFAVNLTTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANNCTFEYVSQPFLM
DLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSETKC
TLKSFTVEKGIYQTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQAGSTPCNGVEGFN
CYFPLQSYGFQPTNGVGYQPYRVVVLSFELLHAPATVCGPKKSTNLVKNKCVNFNFNGLTGT
GVLTESNKKFLPFQQFGRDIADTTDAVRDPQTLEILDITPCSFGGVSVITPGTNTSNQVAVL
YQDVNCTEVPVAIHADQLTPTWRVYSTGSNVFQTRAGCLIGAEHVNNSYECDIPIGAGICAS
YQTQTNSPGSASSVASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQVKQIYKTPPIKDF
GGFNFSQILPDPSKPSKRSFIEDLLFNKVTLADAGFIKQYGDCLGDIAARDLICAQKFNGLT
VLPPLLTDEMIAQYTSALLAGTITSGWTFGAGAALQIPFAMQMAYRFNGIGVTQNVLYENQK
LIANQFNSAIGKIQDSLSSTASALGKLQDVVNQNAQALNTLVKQLSSNFGAISSVLNDILSR
LDPPEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKYFKNHTSPDVDLGDI
SGINASVVNIQKEIDRLNEVAKNLNESLIDLQELGKYEQYIKWPWYIWLGFIAGLIAIVMVT
IMLVVYMVSRDNVSCSICL
(PDI) Modified SARS-CoV-2 S protein GSAS + PP with H5i Hemagglutinin CT (alternative
1) AA
(SEQ ID NO: 59)
MAKNVAIFGLLFSLLVLVPSQIFAVNLTTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANNCTFEYVSQPFLM
DLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSETKC
TLKSFTVEKGIYQTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQAGSTPCNGVEGFN
CYFPLQSYGFQPTNGVGYQPYRVVVLSFELLHAPATVCGPKKSTNLVKNKCVNFNFNGLTGT
GVLTESNKKFLPFQQFGRDIADTTDAVRDPQTLEILDITPCSFGGVSVITPGTNTSNQVAVL
YQDVNCTEVPVAIHADQLTPTWRVYSTGSNVFQTRAGCLIGAEHVNNSYECDIPIGAGICAS
YQTQTNSPGSASSVASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQVKQIYKTPPIKDF
GGFNFSQILPDPSKPSKRSFIEDLLFNKVTLADAGFIKQYGDCLGDIAARDLICAQKFNGLT
VLPPLLTDEMIAQYTSALLAGTITSGWTFGAGAALQIPFAMQMAYRENGIGVTQNVLYENQK
LIANQFNSAIGKIQDSLSSTASALGKLQDVVNQNAQALNTLVKQLSSNFGAISSVLNDILSR
LDPPEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKYFKNHTSPDVDLGDI
SGINASVVNIQKEIDRLNEVAKNLNESLIDLQELGKYEQYIKWPWYIWLGFIAGLIAIVMVT
IMMAGLSLWMCSNGSLQCRICI
(PDI) Modified SARS-CoV-2 S protein GSAS + PP with H5i Hemagglutinin CT (alternative
2) AA
(SEQ ID NO: 60)
MAKNVAIFGLLFSLLVLVPSQIFAVNLTTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANNCTFEYVSQPFLM
DLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSETKC
TLKSFTVEKGIYQTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQAGSTPCNGVEGFN
CYFPLQSYGFQPTNGVGYQPYRVVVLSFELLHAPATVCGPKKSTNLVKNKCVNFNFNGLTGT
GVLTESNKKFLPFQQFGRDIADTTDAVRDPQTLEILDITPCSFGGVSVITPGTNTSNQVAVL
YQDVNCTEVPVAIHADQLTPTWRVYSTGSNVFQTRAGCLIGAEHVNNSYECDIPIGAGICAS
YQTQTNSPGSASSVASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQVKQIYKTPPIKDF
GGFNFSQILPDPSKPSKRSFIEDLLFNKVTLADAGFIKQYGDCLGDIAARDLICAQKFNGLT
VLPPLLTDEMIAQYTSALLAGTITSGWTFGAGAALQIPFAMQMAYRFNGIGVTQNVLYENQK
LIANQFNSAIGKIQDSLSSTASALGKLQDVVNQNAQALNTLVKQLSSNFGAISSVLNDILSR
LDPPEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKYFKNHTSPDVDLGDI
SGINASVVNIQKEIDRLNEVAKNLNESLIDLQELGKYEQYIKWPWYIWLGFIAGLIAIVMVT
IMAGLSLWMCSNGSLQCRICI
(PDI) Modified SARS-CoV-2 S protein GSAS + PP with H5i Hemagglutinin CT (alternative
3) AA
(SEQ ID NO: 61)
MAKNVAIFGLLFSLLVLVPSQIFAVNLTTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANNCTFEYVSQPFLM
DLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSETKC
TLKSFTVEKGIYQTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQAGSTPCNGVEGFN
CYFPLQSYGFQPTNGVGYQPYRVVVLSFELLHAPATVCGPKKSTNLVKNKCVNFNFNGLTGT
GVLTESNKKFLPFQQFGRDIADTTDAVRDPQTLEILDITPCSFGGVSVITPGTNTSNQVAVL
YQDVNCTEVPVAIHADQLTPTWRVYSTGSNVFQTRAGCLIGAEHVNNSYECDIPIGAGICAS
YQTQTNSPGSASSVASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQVKQIYKTPPIKDF
GGFNFSQILPDPSKPSKRSFIEDLLFNKVTLADAGFIKQYGDCLGDIAARDLICAQKFNGLT
VLPPLLTDEMIAQYTSALLAGTITSGWTFGAGAALQIPFAMQMAYRENGIGVTQNVLYENQK
LIANQFNSAIGKIQDSLSSTASALGKLQDVVNQNAQALNTLVKQLSSNFGAISSVLNDILSR
LDPPEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKYFKNHTSPDVDLGDI
SGINASVVNIQKEIDRLNEVAKNLNESLIDLQELGKYEQYIKWPWYIWLGFIAGLIAIVMVT
IMLCCMCSNGSLQCRICI
(PDI) Modified SARS-CoV-2 S protein GSAS + PP with H5i Hemagglutinin CT (alternative
4) AA
(SEQ ID NO: 62)
MAKNVAIFGLLFSLLVLVPSQIFAVNLTTRTQLPPAYTNSFTRGVYYPDKVFRSSVLHSTQD
LFLPFFSNVTWFHAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANNCTFEYVSQPFLM
DLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSETKC
TLKSFTVEKGIYQTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQAGSTPCNGVEGFN
CYFPLQSYGFQPTNGVGYQPYRVVVLSFELLHAPATVCGPKKSTNLVKNKCVNFNFNGLTGT
GVLTESNKKFLPFQQFGRDIADTTDAVRDPQTLEILDITPCSFGGVSVITPGTNTSNQVAVL
YQDVNCTEVPVAIHADQLTPTWRVYSTGSNVFQTRAGCLIGAEHVNNSYECDIPIGAGICAS
YQTQTNSPGSASSVASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQVKQIYKTPPIKDF
GGFNFSQILPDPSKPSKRSFIEDLLFNKVTLADAGFIKQYGDCLGDIAARDLICAQKFNGLT
VLPPLLTDEMIAQYTSALLAGTITSGWTFGAGAALQIPFAMQMAYRFNGIGVTQNVLYENQK
LIANQFNSAIGKIQDSLSSTASALGKLQDVVNQNAQALNTLVKQLSSNFGAISSVLNDILSR
LDPPEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKYFKNHTSPDVDLGDI
SGINASVVNIQKEIDRLNEVAKNLNESLIDLQELGKYEQYIKWPWYIWLGFIAGLIAIVMVT
IMLCCSNGSLQCRICI
N-terminal region of the native SARS-CoV-2 S protein (native signal peptide sequence
underlined)
(SEQ ID NO: 63)
MFVFLVLLPLVSSQCVNLTTRTQLPPAYTNS
Modified TMCT with intervening peptide sequence (X)ₙ
(SEQ ID NO: 64)
WYIWLGFIAGLIAIVMVTIM(X)ₙCSNGSXXCXICI
PDI-S Protein GSAS + 4P-DNA
(SEQ ID NO: 65)
atggcgaaaaacgttgcgattttcggcttattgttttctcttcttgtgttggttccttctca
gatcttcgcggtgaatcttacgacgcgaacacagttaccacccgcatatacaaatagcttca
ctcggggtgtttattaccccgacaaagtgttcaggtcctccgtgctccactcaacacaggac
ctctttcttcctttcttttctaacgtgacatggtttcatgccattcatgtatccggcactaa
cggtactaagaggttcgataatcctgtgctccctttcaatgacggcgtttactttgcaagca
cagagaagagtaacatcatccgaggttggatctttggcactaccctcgattcaaagacgcag
agcctcctcattgtgaacaatgccactaacgtggtgatcaaagtttgcgagtttcagttctg
caatgaccctttcttgggggtgtactatcataagaacaacaagtcttggatggaatctgaat
tccgcgtctatagcagcgccaacaactgcacctttgaatacgtgtcccagcccttccttatg
gacctggagggaaagcagggaaactttaagaatctgagagagttcgtgtttaaaaatatcga
cggctattttaagatctattctaagcacacgcctattaatctcgtgcgcgatcttccacaag
gcttcagcgccctggaaccactcgtggacctcccaattggtatcaacatcactagatttcag
actctgcttgccctccaccgatcctatctgacacccggagactcctctagcggctggactgc
cggcgctgccgcttattacgttggttatcttcagccacgcacgttcctgctgaagtataacg
agaatggtactattaccgatgccgtggattgtgcccttgaccccctgtccgaaactaagtgc
acactcaagtcattcactgtggaaaaaggaatctaccagacaagcaattttcgggtccagcc
tactgagagcattgtgcgctttcctaacatcacaaatctttgccccttcggagaggttttca
atgctacacggtttgcctccgtgtatgcctggaaccgcaagagaatttccaattgcgtggcc
gattactccgtgctctacaatagtgcaagctttagcacctttaagtgctatggcgtatcccc
tactaagcttaacgacttgtgtttcacaaacgtgtatgccgactcctttgtgatacggggcg
acgaagttagacagatagcaccaggacagacgggaaagatagctgactacaactataagctt
cctgatgacttcactggctgcgttatcgcgtggaattctaacaacctggactcaaaagtcgg
cggcaactataactatctctatcggctgttccgcaagagtaaccttaagccctttgagagag
atataagcactgaaatctaccaggctggcagtacgccctgtaatggcgtggaaggctttaat
tgttattttccactgcaatcctatggttttcagccaaccaatggcgtgggctaccaaccata
ccgcgtcgtggtgctctcctttgaactgctccacgctcccgcgactgtctgcggccccaaga
agtccacgaaccttgtgaagaataagtgcgttaattttaatttcaacggcctcactggaaca
ggagtgctcactgagagtaacaagaagttcctgccatttcaacaatttggcagagacatagc
cgatactactgacgccgttagggacccccagaccctcgagattctcgatataacgccctgct
ccttcggtggagtttccgtgatcacgccaggcaccaataccagtaaccaggtcgccgtgctg
tatcaggatgtcaactgtactgaggtgcccgtagccatccatgcggatcagctcacaccaac
ttggagggtgtacagcaccggctccaatgtattccagactcgggccggatgccttattggcg
ccgaacacgtgaacaatagttacgaatgcgatattccaattggcgccggaatctgtgctagc
taccagactcagacgaactccccaggcagcgccagcagcgttgccagccagtcaatcatcgc
ttatacaatgtcacttggagccgaaaactccgtggcttactcaaacaacagcatcgccatcc
ccacaaacttcaccatatccgtgacaactgagattctgccagtgtccatgactaagacgtcc
gtagattgcactatgtacatatgcggcgacagcacagaatgttctaatctgctgctgcaata
tggaagcttctgcactcaactgaacagagcgctcacaggcatcgccgtggagcaggataaga
atacccaggaggtgttcgcccaagttaagcagatctacaagaccccacccataaaggatttc
ggtggattcaattttagtcagatactcccagacccatctaagccatccaagaggagccccat
cgaggatcttttgtttaacaaagttactctggccgacgccggtttcatcaagcagtacggag
attgcctcggcgacatcgctgctcgtgacctcatctgtgcgcaaaagtttaacggtctgacg
gtgctgcctcccctccttactgatgaaatgatcgcccagtataccagcgcactcctcgctgg
caccataacatccggttggacattcggcgctggtgcagcactgcagataccattcgccatgc
aaatggcatatcgtttcaacggtatcggtgtcacacagaatgtcctatatgagaaccagaag
ctgatcgcaaatcagttcaatagtgccatcggaaaaatccaggatagccttagcagcacacc
ctcagcccttggcaaactccaggatgtcgtgaaccagaatgcccaggctctcaataccctcg
tgaagcagctctcatctaatttcggcgcaatttccagtgtcctcaacgacatcctcagccgc
ctcgacccccccgaggccgaagtgcagattgacagactgattacaggtcgactccagagcct
ccagacttacgtgactcagcagctgataagagccgccgagataagggccagcgctaacctgg
ctgccacaaagatgtctgagtgcgtgctgggccagtccaagagagtagacttctgtggcaaa
ggctaccatctgatgagcttcccacaatccgcacctcacggcgtagtgttcctccacgtgac
atatgtaccggctcaggagaagaatttcactaccgctcctgctatatgccatgatggaaagg
ctcacttcccccgggagggggtgttcgtgtccaacggcacccattggtttgtgactcagcgg
aatttctacgaaccccagatcataaccactgacaacacatttgtgtccggaaattgtgacgt
ggtcattggaatagtgaacaacactgtttatgatccactgcagccagaacttgacagcttta
aggaggagctcgacaagtacttcaagaatcatacgtcaccagatgtggacctcggagatatt
agcggtatcaatgccagtgttgtcaatattcagaaggaaatagaccgccttaatgaggtcgc
caaaaatctgaacgagagcctcatcgatcttcaggagctgggcaaatatgagcagtacatca
agtggccttggtatatttggcttggcttcatcgccggcctgatcgccatagtaatggtcaca
attatgctctctttatggatgtgctccaatggatcgttacaatgcagaatttgcatttaa
PDI-S Protein GSAS + 6P-DNA
(SEQ ID NO: 66)
atggcgaaaaacgttgcgattttcggcttattgttttctcttcttgtgttggttccttctca
gatcttcgcggtgaatcttacgacgcgaacacagttaccacccgcatatacaaatagcttca
ctcggggtgtttattaccccgacaaagtgttcaggtcctccgtgctccactcaacacaggac
ctctttcttcctttcttttctaacgtgacatggtttcatgccattcatgtatccggcactaa
cggtactaagaggttcgataatcctgtgctccctttcaatgacggcgtttactttgcaagca
cagagaagagtaacatcatccgaggttggatctttggcactaccctcgattcaaagacgcag
agcctcctcattgtgaacaatgccactaacgtggtgatcaaagtttgcgagtttcagttctg
caatgaccctttcttgggggtgtactatcataagaacaacaagtcttggatggaatctgaat
tccgcgtctatagcagcgccaacaactgcacctttgaatacgtgtcccagcccttccttatg
gacctggagggaaagcagggaaactttaagaatctgagagagttcgtgtttaaaaatatcga
cggctattttaagatctattctaagcacacgcctattaatctcgtgcgcgatcttccacaag
gcttcagcgccctggaaccactcgtggacctcccaattggtatcaacatcactagatttcag
actctgcttgccctccaccgatcctatctgacacccggagactcctctagcggctggactgc
cggcgctgccgcttattacgttggttatcttcagccacgcacgttcctgctgaagtataacg
agaatggtactattaccgatgccgtggattgtgcccttgaccccctgtccgaaactaagtgc
acactcaagtcattcactgtggaaaaaggaatctaccagacaagcaattttcgggtccagcc
tactgagagcattgtgcgctttcctaacatcacaaatctttgccccttcggagaggttttca
atgctacacggtttgcctccgtgtatgcctggaaccgcaagagaatttccaattgcgtggcc
gattactccgtgctctacaatagtgcaagctttagcacctttaagtgctatggcgtatcccc
tactaagcttaacgacttgtgtttcacaaacgtgtatgccgactcctttgtgatacggggcg
acgaagttagacagatagcaccaggacagacgggaaagatagctgactacaactataagctt
cctgatgacttcactggctgcgttatcgcgtggaattctaacaacctggactcaaaagtcgg
cggcaactataactatctctatcggctgttccgcaagagtaaccttaagccctttgagagag
atataagcactgaaatctaccaggctggcagtacgccctgtaatggcgtggaaggctttaat
tgttattttccactgcaatcctatggttttcagccaaccaatggcgtgggctaccaaccata
ccgcgtcgtggtgctctcctttgaactgctccacgctcccgcgactgtctgcggccccaaga
agtccacgaaccttgtgaagaataagtgcgttaattttaatttcaacggcctcactggaaca
ggagtgctcactgagagtaacaagaagttcctgccatttcaacaatttggcagagacatagc
cgatactactgacgccgttagggacccccagaccctcgagattctcgatataacgccctgct
ccttcggtggagtttccgtgatcacgccaggcaccaataccagtaaccaggtcgccgtgctg
tatcaggatgtcaactgtactgaggtgcccgtagccatccatgcggatcagctcacaccaac
ttggagggtgtacagcaccggctccaatgtattccagactcgggccggatgccttattggcg
ccgaacacgtgaacaatagttacgaatgcgatattccaattggcgccggaatctgtgctagc
taccagactcagacgaactccccaggcagcgccagcagcgttgccagccagtcaatcatcgc
ttatacaatgtcacttggagccgaaaactccgtggcttactcaaacaacagcatcgccatcc
ccacaaacttcaccatatccgtgacaactgagattctgccagtgtccatgactaagacgtcc
gtagattgcactatgtacatatgcggcgacagcacagaatgttctaatctgctgctgcaata
tggaagcttctgcactcaactgaacagagcgctcacaggcatcgccgtggagcaggataaga
atacccaggaggtgttcgcccaagttaagcagatctacaagaccccacccataaaggatttc
ggtggattcaattttagtcagatactcccagacccatctaagccatccaagaggagccccat
cgaggatcttttgtttaacaaagttactctggccgacgccggtttcatcaagcagtacggag
attgcctcggcgacatcgctgctcgtgacctcatctgtgcgcaaaagtttaacggtctgacg
gtgctgcctcccctccttactgatgaaatgatcgcccagtataccagcgcactcctcgctgg
caccataacatccggttggacattcggcgctggtcccgcactgcagataccattccccatgc
aaatggcatatcgtttcaacggtatcggtgtcacacagaatgtcctatatgagaaccagaag
ctgatcgcaaatcagttcaatagtgccatcggaaaaatccaggatagccttagcagcacacc
ctcagcccttggcaaactccaggatgtcgtgaaccagaatgcccaggctctcaataccctcg
tgaagcagctctcatctaatttcggcgcaatttccagtgtcctcaacgacatcctcagccgc
ctcgacccccccgaggccgaagtgcagattgacagactgattacaggtcgactccagagcct
ccagacttacgtgactcagcagctgataagagccgccgagataagggccagcgctaacctgg
ctgccacaaagatgtctgagtgcgtgctgggccagtccaagagagtagacttctgtggcaaa
ggctaccatctgatgagcttcccacaatccgcacctcacggcgtagtgttcctccacgtgac
atatgtaccggctcaggagaagaatttcactaccgctcctgctatatgccatgatggaaagg
ctcacttcccccgggagggggtgttcgtgtccaacggcacccattggtttgtgactcagcgg
aatttctacgaaccccagatcataaccactgacaacacatttgtgtccggaaattgtgacgt
ggtcattggaatagtgaacaacactgtttatgatccactgcagccagaacttgacagcttta
aggaggagctcgacaagtacttcaagaatcatacgtcaccagatgtggacctcggagatatt
agcggtatcaatgccagtgttgtcaatattcagaaggaaatagaccgccttaatgaggtcgc
caaaaatctgaacgagagcctcatcgatcttcaggagctgggcaaatatgagcagtacatca
agtggccttggtatatttggcttggcttcatcgccggcctgatcgccatagtaatggtcaca
attatgctctctttatggatgtgctccaatggatcgttacaatgcagaatttgcatttaa
PDI-S Protein GSAS + 2P + L923F-DNA
(SEQ ID NO: 67)
atggcgaaaaacgttgcgattttcggcttattgttttctcttcttgtgttggttccttctca
gatcttcgcggtgaatcttacgacgcgaacacagttaccacccgcatatacaaatagcttca
ctcggggtgtttattaccccgacaaagtgttcaggtcctccgtgctccactcaacacaggac
ctctttcttcctttcttttctaacgtgacatggtttcatgccattcatgtatccggcactaa
cggtactaagaggttcgataatcctgtgctccctttcaatgacggcgtttactttgcaagca
cagagaagagtaacatcatccgaggttggatctttggcactaccctcgattcaaagacgcag
agcctcctcattgtgaacaatgccactaacgtggtgatcaaagtttgcgagtttcagttctg
caatgaccctttcttgggggtgtactatcataagaacaacaagtcttggatggaatctgaat
tccgcgtctatagcagcgccaacaactgcacctttgaatacgtgtcccagcccttccttatg
gacctggagggaaagcagggaaactttaagaatctgagagagttcgtgtttaaaaatatcga
cggctattttaagatctattctaagcacacgcctattaatctcgtgcgcgatcttccacaag
gcttcagcgccctggaaccactcgtggacctcccaattggtatcaacatcactagatttcag
actctgcttgccctccaccgatcctatctgacacccggagactcctctagcggctggactgc
cggcgctgccgcttattacgttggttatcttcagccacgcacgttcctgctgaagtataacg
agaatggtactattaccgatgccgtggattgtgcccttgaccccctgtccgaaactaagtgc
acactcaagtcattcactgtggaaaaaggaatctaccagacaagcaattttcgggtccagcc
tactgagagcattgtgcgctttcctaacatcacaaatctttgccccttcggagaggttttca
atgctacacggtttgcctccgtgtatgcctggaaccgcaagagaatttccaattgcgtggcc
gattactccgtgctctacaatagtgcaagctttagcacctttaagtgctatggcgtatcccc
tactaagcttaacgacttgtgtttcacaaacgtgtatgccgactcctttgtgatacggggcg
acgaagttagacagatagcaccaggacagacgggaaagatagctgactacaactataagctt
cctgatgacttcactggctgcgttatcgcgtggaattctaacaacctggactcaaaagtcgg
cggcaactataactatctctatcggctgttccgcaagagtaaccttaagccctttgagagag
atataagcactgaaatctaccaggctggcagtacgccctgtaatggcgtggaaggctttaat
tgttattttccactgcaatcctatggttttcagccaaccaatggcgtgggctaccaaccata
ccgcgtcgtggtgctctcctttgaactgctccacgctcccgcgactgtctgcggccccaaga
agtccacgaaccttgtgaagaataagtgcgttaattttaatttcaacggcctcactggaaca
ggagtgctcactgagagtaacaagaagttcctgccatttcaacaatttggcagagacatagc
cgatactactgacgccgttagggacccccagaccctcgagattctcgatataacgccctgct
ccttcggtggagtttccgtgatcacgccaggcaccaataccagtaaccaggtcgccgtgctg
tatcaggatgtcaactgtactgaggtgcccgtagccatccatgcggatcagctcacaccaac
ttggagggtgtacagcaccggctccaatgtattccagactcgggccggatgccttattggcg
ccgaacacgtgaacaatagttacgaatgcgatattccaattggcgccggaatctgtgctagc
taccagactcagacgaactccccaggcagcgccagcagcgttgccagccagtcaatcatcgc
ttatacaatgtcacttggagccgaaaactccgtggcttactcaaacaacagcatcgccatcc
ccacaaacttcaccatatccgtgacaactgagattctgccagtgtccatgactaagacgtcc
gtagattgcactatgtacatatgcggcgacagcacagaatgttctaatctgctgctgcaata
tggaagcttctgcactcaactgaacagagcgctcacaggcatcgccgtggagcaggataaga
atacccaggaggtgttcgcccaagttaagcagatctacaagaccccacccataaaggatttc
ggtggattcaattttagtcagatactcccagacccatctaagccatccaagaggagctttat
cgaggatcttttgtttaacaaagttactctggccgacgccggtttcatcaagcagtacggag
attgcctcggcgacatcgctgctcgtgacctcatctgtgcgcaaaagtttaacggtctgacg
gtgctgcctcccctccttactgatgaaatgatcgcccagtataccagcgcactcctcgctgg
caccataacatccggttggacattcggcgctggtgcagcactgcagataccattcgccatgc
aaatggcatatcgtttcaacggtatcggtgtcacacagaatgtcctatatgagaaccagaag
ctgatcgcaaatcagttcaatagtgccatcggaaaaatccaggatagcttcagcagcacagc
ctcagcccttggcaaactccaggatgtcgtgaaccagaatgcccaggctctcaataccctcg
tgaagcagctctcatctaatttcggcgcaatttccagtgtcctcaacgacatcctcagccgc
ctcgacccccccgaggccgaagtgcagattgacagactgattacaggtcgactccagagcct
ccagacttacgtgactcagcagctgataagagccgccgagataagggccagcgctaacctgg
ctgccacaaagatgtctgagtgcgtgctgggccagtccaagagagtagacttctgtggcaaa
ggctaccatctgatgagcttcccacaatccgcacctcacggcgtagtgttcctccacgtgac
atatgtaccggctcaggagaagaatttcactaccgctcctgctatatgccatgatggaaagg
ctcacttcccccgggagggggtgttcgtgtccaacggcacccattggtttgtgactcagcgg
aatttctacgaaccccagatcataaccactgacaacacatttgtgtccggaaattgtgacgt
ggtcattggaatagtgaacaacactgtttatgatccactgcagccagaacttgacagcttta
aggaggagctcgacaagtacttcaagaatcatacgtcaccagatgtggacctcggagatatt
agcggtatcaatgccagtgttgtcaatattcagaaggaaatagaccgccttaatgaggtcgc
caaaaatctgaacgagagcctcatcgatcttcaggagctgggcaaatatgagcagtacatca
agtggccttggtatatttggcttggcttcatcgccggcctgatcgccatagtaatggtcaca
attatgctctctttatggatgtgctccaatggatcgttacaatgcagaatttgcatttaa
PDI-S Protein GSAS + 4P + L923F-DNA
(SEQ ID NO: 68)
atggcgaaaaacgttgcgattttcggcttattgttttctcttcttgtgttggttccttctca
gatcttcgcggtgaatcttacgacgcgaacacagttaccacccgcatatacaaatagcttca
ctcggggtgtttattaccccgacaaagtgttcaggtcctccgtgctccactcaacacaggac
ctctttcttcctttcttttctaacgtgacatggtttcatgccattcatgtatccggcactaa
cggtactaagaggttcgataatcctgtgctccctttcaatgacggcgtttactttgcaagca
cagagaagagtaacatcatccgaggttggatctttggcactaccctcgattcaaagacgcag
agcctcctcattgtgaacaatgccactaacgtggtgatcaaagtttgcgagtttcagttctg
caatgaccctttcttgggggtgtactatcataagaacaacaagtcttggatggaatctgaat
tccgcgtctatagcagcgccaacaactgcacctttgaatacgtgtcccagcccttccttatg
gacctggagggaaagcagggaaactttaagaatctgagagagttcgtgtttaaaaatatcga
cggctattttaagatctattctaagcacacgcctattaatctcgtgcgcgatcttccacaag
gcttcagcgccctggaaccactcgtggacctcccaattggtatcaacatcactagatttcag
actctgcttgccctccaccgatcctatctgacacccggagactcctctagcggctggactgc
cggcgctgccgcttattacgttggttatcttcagccacgcacgttcctgctgaagtataacg
agaatggtactattaccgatgccgtggattgtgcccttgaccccctgtccgaaactaagtgc
acactcaagtcattcactgtggaaaaaggaatctaccagacaagcaattttcgggtccagcc
tactgagagcattgtgcgctttcctaacatcacaaatctttgccccttcggagaggttttca
atgctacacggtttgcctccgtgtatgcctggaaccgcaagagaatttccaattgcgtggcc
gattactccgtgctctacaatagtgcaagctttagcacctttaagtgctatggcgtatcccc
tactaagcttaacgacttgtgtttcacaaacgtgtatgccgactcctttgtgatacggggcg
acgaagttagacagatagcaccaggacagacgggaaagatagctgactacaactataagctt
cctgatgacttcactggctgcgttatcgcgtggaattctaacaacctggactcaaaagtcgg
cggcaactataactatctctatcggctgttccgcaagagtaaccttaagccctttgagagag
atataagcactgaaatctaccaggctggcagtacgccctgtaatggcgtggaaggctttaat
tgttattttccactgcaatcctatggttttcagccaaccaatggcgtgggctaccaaccata
ccgcgtcgtggtgctctcctttgaactgctccacgctcccgcgactgtctgcggccccaaga
agtccacgaaccttgtgaagaataagtgcgttaattttaatttcaacggcctcactggaaca
ggagtgctcactgagagtaacaagaagttcctgccatttcaacaatttggcagagacatagc
cgatactactgacgccgttagggacccccagaccctcgagattctcgatataacgccctgct
ccttcggtggagtttccgtgatcacgccaggcaccaataccagtaaccaggtcgccgtgctg
tatcaggatgtcaactgtactgaggtgcccgtagccatccatgcggatcagctcacaccaac
ttggagggtgtacagcaccggctccaatgtattccagactcgggccggatgccttattggcg
ccgaacacgtgaacaatagttacgaatgcgatattccaattggcgccggaatctgtgctagc
taccagactcagacgaactccccaggcagcgccagcagcgttgccagccagtcaatcatcgc
ttatacaatgtcacttggagccgaaaactccgtggcttactcaaacaacagcatcgccatcc
ccacaaacttcaccatatccgtgacaactgagattctgccagtgtccatgactaagacgtcc
gtagattgcactatgtacatatgcggcgacagcacagaatgttctaatctgctgctgcaata
tggaagcttctgcactcaactgaacagagcgctcacaggcatcgccgtggagcaggataaga
atacccaggaggtgttcgcccaagttaagcagatctacaagaccccacccataaaggatttc
ggtggattcaattttagtcagatactcccagacccatctaagccatccaagaggagccccat
cgaggatcttttgtttaacaaagttactctggccgacgccggtttcatcaagcagtacggag
attgcctcggcgacatcgctgctcgtgacctcatctgtgcgcaaaagtttaacggtctgacg
gtgctgcctcccctccttactgatgaaatgatcgcccagtataccagcgcactcctcgctgg
caccataacatccggttggacattcggcgctggtgcagcactgcagataccattcgccatgc
aaatggcatatcgtttcaacggtatcggtgtcacacagaatgtcctatatgagaaccagaag
ctgatcgcaaatcagttcaatagtgccatcggaaaaatccaggatagcttcagcagcacacc
ctcagcccttggcaaactccaggatgtcgtgaaccagaatgcccaggctctcaataccctcg
tgaagcagctctcatctaatttcggcgcaatttccagtgtcctcaacgacatcctcagccgc
ctcgacccccccgaggccgaagtgcagattgacagactgattacaggtcgactccagagcct
ccagacttacgtgactcagcagctgataagagccgccgagataagggccagcgctaacctgg
ctgccacaaagatgtctgagtgcgtgctgggccagtccaagagagtagacttctgtggcaaa
ggctaccatctgatgagcttcccacaatccgcacctcacggcgtagtgttcctccacgtgac
atatgtaccggctcaggagaagaatttcactaccgctcctgctatatgccatgatggaaagg
ctcacttcccccgggagggggtgttcgtgtccaacggcacccattggtttgtgactcagcgg
aatttctacgaaccccagatcataaccactgacaacacatttgtgtccggaaattgtgacgt
ggtcattggaatagtgaacaacactgtttatgatccactgcagccagaacttgacagcttta
aggaggagctcgacaagtacttcaagaatcatacgtcaccagatgtggacctcggagatatt
agcggtatcaatgccagtgttgtcaatattcagaaggaaatagaccgccttaatgaggtcgc
caaaaatctgaacgagagcctcatcgatcttcaggagctgggcaaatatgagcagtacatca
agtggccttggtatatttggcttggcttcatcgccggcctgatcgccatagtaatggtcaca
attatgctctctttatggatgtgctccaatggatcgttacaatgcagaatttgcatttaa
PDI-S Protein GSAS + 6P + L923F-DNA
(SEQ ID NO: 69)
atggcgaaaaacgttgcgattttcggcttattgttttctcttcttgtgttggttccttctca
gatcttcgcggtgaatcttacgacgcgaacacagttaccacccgcatatacaaatagcttca
ctcggggtgtttattaccccgacaaagtgttcaggtcctccgtgctccactcaacacaggac
ctctttcttcctttcttttctaacgtgacatggtttcatgccattcatgtatccggcactaa
cggtactaagaggttcgataatcctgtgctccctttcaatgacggcgtttactttgcaagca
cagagaagagtaacatcatccgaggttggatctttggcactaccctcgattcaaagacgcag
agcctcctcattgtgaacaatgccactaacgtggtgatcaaagtttgcgagtttcagttctg
caatgaccctttcttgggggtgtactatcataagaacaacaagtcttggatggaatctgaat
tccgcgtctatagcagcgccaacaactgcacctttgaatacgtgtcccagcccttccttatg
gacctggagggaaagcagggaaactttaagaatctgagagagttcgtgtttaaaaatatcga
cggctattttaagatctattctaagcacacgcctattaatctcgtgcgcgatcttccacaag
gcttcagcgccctggaaccactcgtggacctcccaattggtatcaacatcactagatttcag
actctgcttgccctccaccgatcctatctgacacccggagactcctctagcggctggactgc
cggcgctgccgcttattacgttggttatcttcagccacgcacgttcctgctgaagtataacg
agaatggtactattaccgatgccgtggattgtgcccttgaccccctgtccgaaactaagtgc
acactcaagtcattcactgtggaaaaaggaatctaccagacaagcaattttcgggtccagcc
tactgagagcattgtgcgctttcctaacatcacaaatctttgccccttcggagaggttttca
atgctacacggtttgcctccgtgtatgcctggaaccgcaagagaatttccaattgcgtggcc
gattactccgtgctctacaatagtgcaagctttagcacctttaagtgctatggcgtatcccc
tactaagcttaacgacttgtgtttcacaaacgtgtatgccgactcctttgtgatacggggcg
acgaagttagacagatagcaccaggacagacgggaaagatagctgactacaactataagctt
cctgatgacttcactggctgcgttatcgcgtggaattctaacaacctggactcaaaagtcgg
cggcaactataactatctctatcggctgttccgcaagagtaaccttaagccctttgagagag
atataagcactgaaatctaccaggctggcagtacgccctgtaatggcgtggaaggctttaat
tgttattttccactgcaatcctatggttttcagccaaccaatggcgtgggctaccaaccata
ccgcgtcgtggtgctctcctttgaactgctccacgctcccgcgactgtctgcggccccaaga
agtccacgaaccttgtgaagaataagtgcgttaattttaatttcaacggcctcactggaaca
ggagtgctcactgagagtaacaagaagttcctgccatttcaacaatttggcagagacatagc
cgatactactgacgccgttagggacccccagaccctcgagattctcgatataacgccctgct
ccttcggtggagtttccgtgatcacgccaggcaccaataccagtaaccaggtcgccgtgctg
tatcaggatgtcaactgtactgaggtgcccgtagccatccatgcggatcagctcacaccaac
ttggagggtgtacagcaccggctccaatgtattccagactcgggccggatgccttattggcg
ccgaacacgtgaacaatagttacgaatgcgatattccaattggcgccggaatctgtgctagc
taccagactcagacgaactccccaggcagcgccagcagcgttgccagccagtcaatcatcgc
ttatacaatgtcacttggagccgaaaactccgtggcttactcaaacaacagcatcgccatcc
ccacaaacttcaccatatccgtgacaactgagattctgccagtgtccatgactaagacgtcc
gtagattgcactatgtacatatgcggcgacagcacagaatgttctaatctgctgctgcaata
tggaagcttctgcactcaactgaacagagcgctcacaggcatcgccgtggagcaggataaga
atacccaggaggtgttcgcccaagttaagcagatctacaagaccccacccataaaggatttc
ggtggattcaattttagtcagatactcccagacccatctaagccatccaagaggagccccat
cgaggatcttttgtttaacaaagttactctggccgacgccggtttcatcaagcagtacggag
attgcctcggcgacatcgctgctcgtgacctcatctgtgcgcaaaagtttaacggtctgacg
gtgctgcctcccctccttactgatgaaatgatcgcccagtataccagcgcactcctcgctgg
caccataacatccggttggacattcggcgctggtcccgcactgcagataccattccccatgc
aaatggcatatcgtttcaacggtatcggtgtcacacagaatgtcctatatgagaaccagaag
ctgatcgcaaatcagttcaatagtgccatcggaaaaatccaggatagcttcagcagcacacc
ctcagcccttggcaaactccaggatgtcgtgaaccagaatgcccaggctctcaataccctcg
tgaagcagctctcatctaatttcggcgcaatttccagtgtcctcaacgacatcctcagccgc
ctcgacccccccgaggccgaagtgcagattgacagactgattacaggtcgactccagagcct
ccagacttacgtgactcagcagctgataagagccgccgagataagggccagcgctaacctgg
ctgccacaaagatgtctgagtgcgtgctgggccagtccaagagagtagacttctgtggcaaa
ggctaccatctgatgagcttcccacaatccgcacctcacggcgtagtgttcctccacgtgac
atatgtaccggctcaggagaagaatttcactaccgctcctgctatatgccatgatggaaagg
ctcacttcccccgggagggggtgttcgtgtccaacggcacccattggtttgtgactcagcgg
aatttctacgaaccccagatcataaccactgacaacacatttgtgtccggaaattgtgacgt
ggtcattggaatagtgaacaacactgtttatgatccactgcagccagaacttgacagcttta
aggaggagctcgacaagtacttcaagaatcatacgtcaccagatgtggacctcggagatatt
agcggtatcaatgccagtgttgtcaatattcagaaggaaatagaccgccttaatgaggtcgc
caaaaatctgaacgagagcctcatcgatcttcaggagctgggcaaatatgagcagtacatca
agtggccttggtatatttggcttggcttcatcgccggcctgatcgccatagtaatggtcaca
attatgctctctttatggatgtgctccaatggatcgttacaatgcagaatttgcatttaa
PDI-Modified S protein with H5i Hemagglutinin CT (V1) DNA
(SEQ ID NO: 70)
atggcgaaaaacgttgcgattttcggcttattgttttctcttcttgtgttggttccttctca
gatcttcgcggtgaatcttacgacgcgaacacagttaccacccgcatatacaaatagcttca
ctcggggtgtttattaccccgacaaagtgttcaggtcctccgtgctccactcaacacaggac
ctctttcttcctttcttttctaacgtgacatggtttcatgccattcatgtatccggcactaa
cggtactaagaggttcgataatcctgtgctccctttcaatgacggcgtttactttgcaagca
cagagaagagtaacatcatccgaggttggatctttggcactaccctcgattcaaagacgcag
agcctcctcattgtgaacaatgccactaacgtggtgatcaaagtttgcgagtttcagttctg
caatgaccctttcttgggggtgtactatcataagaacaacaagtcttggatggaatctgaat
tccgcgtctatagcagcgccaacaactgcacctttgaatacgtgtcccagcccttccttatg
gacctggagggaaagcagggaaactttaagaatctgagagagttcgtgtttaaaaatatcga
cggctattttaagatctattctaagcacacgcctattaatctcgtgcgcgatcttccacaag
gcttcagcgccctggaaccactcgtggacctcccaattggtatcaacatcactagatttcag
actctgcttgccctccaccgatcctatctgacacccggagactcctctagcggctggactgc
cggcgctgccgcttattacgttggttatcttcagccacgcacgttcctgctgaagtataacg
agaatggtactattaccgatgccgtggattgtgcccttgaccccctgtccgaaactaagtgc
acactcaagtcattcactgtggaaaaaggaatctaccagacaagcaattttcgggtccagcc
tactgagagcattgtgcgctttcctaacatcacaaatctttgccccttcggagaggttttca
atgctacacggtttgcctccgtgtatgcctggaaccgcaagagaatttccaattgcgtggcc
gattactccgtgctctacaatagtgcaagctttagcacctttaagtgctatggcgtatcccc
tactaagcttaacgacttgtgtttcacaaacgtgtatgccgactcctttgtgatacggggcg
acgaagttagacagatagcaccaggacagacgggaaagatagctgactacaactataagctt
cctgatgacttcactggctgcgttatcgcgtggaattctaacaacctggactcaaaagtcgg
cggcaactataactatctctatcggctgttccgcaagagtaaccttaagccctttgagagag
atataagcactgaaatctaccaggctggcagtacgccctgtaatggcgtggaaggctttaat
tgttattttccactgcaatcctatggttttcagccaaccaatggcgtgggctaccaaccata
ccgcgtcgtggtgctctcctttgaactgctccacgctcccgcgactgtctgcggccccaaga
agtccacgaaccttgtgaagaataagtgcgttaattttaatttcaacggcctcactggaaca
ggagtgctcactgagagtaacaagaagttcctgccatttcaacaatttggcagagacatagc
cgatactactgacgccgttagggacccccagaccctcgagattctcgatataacgccctgct
ccttcggtggagtttccgtgatcacgccaggcaccaataccagtaaccaggtcgccgtgctg
tatcaggatgtcaactgtactgaggtgcccgtagccatccatgcggatcagctcacaccaac
ttggagggtgtacagcaccggctccaatgtattccagactcgggccggatgccttattggcg
ccgaacacgtgaacaatagttacgaatgcgatattccaattggcgccggaatctgtgctagc
taccagactcagacgaactccccaggcagcgccagcagcgttgccagccagtcaatcatcgc
ttatacaatgtcacttggagccgaaaactccgtggcttactcaaacaacagcatcgccatcc
ccacaaacttcaccatatccgtgacaactgagattctgccagtgtccatgactaagacgtcc
gtagattgcactatgtacatatgcggcgacagcacagaatgttctaatctgctgctgcaata
tggaagcttctgcactcaactgaacagagcgctcacaggcatcgccgtggagcaggataaga
atacccaggaggtgttcgcccaagttaagcagatctacaagaccccacccataaaggatttc
ggtggattcaattttagtcagatactcccagacccatctaagccatccaagaggagctttat
cgaggatcttttgtttaacaaagttactctggccgacgccggtttcatcaagcagtacggag
attgcctcggcgacatcgctgctcgtgacctcatctgtgcgcaaaagtttaacggtctgacg
gtgctgcctcccctccttactgatgaaatgatcgcccagtataccagcgcactcctcgctgg
caccataacatccggttggacattcggcgctggtgcagcactgcagataccattcgccatgc
aaatggcatatcgtttcaacggtatcggtgtcacacagaatgtcctatatgagaaccagaag
ctgatcgcaaatcagttcaatagtgccatcggaaaaatccaggatagccttagcagcacagc
ctcagcccttggcaaactccaggatgtcgtgaaccagaatgcccaggctctcaataccctcg
tgaagcagctctcatctaatttcggcgcaatttccagtgtcctcaacgacatcctcagccgc
ctcgacccccccgaggccgaagtgcagattgacagactgattacaggtcgactccagagcct
ccagacttacgtgactcagcagctgataagagccgccgagataagggccagcgctaacctgg
ctgccacaaagatgtctgagtgcgtgctgggccagtccaagagagtagacttctgtggcaaa
ggctaccatctgatgagcttcccacaatccgcacctcacggcgtagtgttcctccacgtgac
atatgtaccggctcaggagaagaatttcactaccgctcctgctatatgccatgatggaaagg
ctcacttcccccgggagggggtgttcgtgtccaacggcacccattggtttgtgactcagcgg
aatttctacgaaccccagatcataaccactgacaacacatttgtgtccggaaattgtgacgt
ggtcattggaatagtgaacaacactgtttatgatccactgcagccagaacttgacagcttta
aggaggagctcgacaagtacttcaagaatcatacgtcaccagatgtggacctcggagatatt
agcggtatcaatgccagtgttgtcaatattcagaaggaaatagaccgccttaatgaggtcgc
caaaaatctgaacgagagcctcatcgatcttcaggagctgggcaaatatgagcagtacatca
agtggccttggtatatttggcttggcttcatcgccggcctgatcgccatagtaatggtcaca
attatgatggccggcctctctttatggatgtgctccaatggatcgttacaatgcagaatttg
catttaa
PDI-Modified S protein with H5i Hemagglutinin CT (V2) DNA
(SEQ ID NO: 71)
atggcgaaaaacgttgcgattttcggcttattgttttctcttcttgtgttggttccttctca
gatcttcgcggtgaatcttacgacgcgaacacagttaccacccgcatatacaaatagcttca
ctcggggtgtttattaccccgacaaagtgttcaggtcctccgtgctccactcaacacaggac
ctctttcttcctttcttttctaacgtgacatggtttcatgccattcatgtatccggcactaa
cggtactaagaggttcgataatcctgtgctccctttcaatgacggcgtttactttgcaagca
cagagaagagtaacatcatccgaggttggatctttggcactaccctcgattcaaagacgcag
agcctcctcattgtgaacaatgccactaacgtggtgatcaaagtttgcgagtttcagttctg
caatgaccctttcttgggggtgtactatcataagaacaacaagtcttggatggaatctgaat
tccgcgtctatagcagcgccaacaactgcacctttgaatacgtgtcccagcccttccttatg
gacctggagggaaagcagggaaactttaagaatctgagagagttcgtgtttaaaaatatcga
cggctattttaagatctattctaagcacacgcctattaatctcgtgcgcgatcttccacaag
gcttcagcgccctggaaccactcgtggacctcccaattggtatcaacatcactagatttcag
actctgcttgccctccaccgatcctatctgacacccggagactcctctagcggctggactgc
cggcgctgccgcttattacgttggttatcttcagccacgcacgttcctgctgaagtataacg
agaatggtactattaccgatgccgtggattgtgcccttgaccccctgtccgaaactaagtgc
acactcaagtcattcactgtggaaaaaggaatctaccagacaagcaattttcgggtccagcc
tactgagagcattgtgcgctttcctaacatcacaaatctttgccccttcggagaggttttca
atgctacacggtttgcctccgtgtatgcctggaaccgcaagagaatttccaattgcgtggcc
gattactccgtgctctacaatagtgcaagctttagcacctttaagtgctatggcgtatcccc
tactaagcttaacgacttgtgtttcacaaacgtgtatgccgactcctttgtgatacggggcg
acgaagttagacagatagcaccaggacagacgggaaagatagctgactacaactataagctt
cctgatgacttcactggctgcgttatcgcgtggaattctaacaacctggactcaaaagtcgg
cggcaactataactatctctatcggctgttccgcaagagtaaccttaagccctttgagagag
atataagcactgaaatctaccaggctggcagtacgccctgtaatggcgtggaaggctttaat
tgttattttccactgcaatcctatggttttcagccaaccaatggcgtgggctaccaaccata
ccgcgtcgtggtgctctcctttgaactgctccacgctcccgcgactgtctgcggccccaaga
agtccacgaaccttgtgaagaataagtgcgttaattttaatttcaacggcctcactggaaca
ggagtgctcactgagagtaacaagaagttcctgccatttcaacaatttggcagagacatagc
cgatactactgacgccgttagggacccccagaccctcgagattctcgatataacgccctgct
ccttcggtggagtttccgtgatcacgccaggcaccaataccagtaaccaggtcgccgtgctg
tatcaggatgtcaactgtactgaggtgcccgtagccatccatgcggatcagctcacaccaac
ttggagggtgtacagcaccggctccaatgtattccagactcgggccggatgccttattggcg
ccgaacacgtgaacaatagttacgaatgcgatattccaattggcgccggaatctgtgctagc
taccagactcagacgaactccccaggcagcgccagcagcgttgccagccagtcaatcatcgc
ttatacaatgtcacttggagccgaaaactccgtggcttactcaaacaacagcatcgccatcc
ccacaaacttcaccatatccgtgacaactgagattctgccagtgtccatgactaagacgtcc
gtagattgcactatgtacatatgcggcgacagcacagaatgttctaatctgctgctgcaata
tggaagcttctgcactcaactgaacagagcgctcacaggcatcgccgtggagcaggataaga
atacccaggaggtgttcgcccaagttaagcagatctacaagaccccacccataaaggatttc
ggtggattcaattttagtcagatactcccagacccatctaagccatccaagaggagctttat
cgaggatcttttgtttaacaaagttactctggccgacgccggtttcatcaagcagtacggag
attgcctcggcgacatcgctgctcgtgacctcatctgtgcgcaaaagtttaacggtctgacg
gtgctgcctcccctccttactgatgaaatgatcgcccagtataccagcgcactcctcgctgg
caccataacatccggttggacattcggcgctggtgcagcactgcagataccattcgccatgc
aaatggcatatcgtttcaacggtatcggtgtcacacagaatgtcctatatgagaaccagaag
ctgatcgcaaatcagttcaatagtgccatcggaaaaatccaggatagccttagcagcacagc
ctcagcccttggcaaactccaggatgtcgtgaaccagaatgcccaggctctcaataccctcg
tgaagcagctctcatctaatttcggcgcaatttccagtgtcctcaacgacatcctcagccgc
ctcgacccccccgaggccgaagtgcagattgacagactgattacaggtcgactccagagcct
ccagacttacgtgactcagcagctgataagagccgccgagataagggccagcgctaacctgg
ctgccacaaagatgtctgagtgcgtgctgggccagtccaagagagtagacttctgtggcaaa
ggctaccatctgatgagcttcccacaatccgcacctcacggcgtagtgttcctccacgtgac
atatgtaccggctcaggagaagaatttcactaccgctcctgctatatgccatgatggaaagg
ctcacttcccccgggagggggtgttcgtgtccaacggcacccattggtttgtgactcagcgg
aatttctacgaaccccagatcataaccactgacaacacatttgtgtccggaaattgtgacgt
ggtcattggaatagtgaacaacactgtttatgatccactgcagccagaacttgacagcttta
aggaggagctcgacaagtacttcaagaatcatacgtcaccagatgtggacctcggagatatt
agcggtatcaatgccagtgttgtcaatattcagaaggaaatagaccgccttaatgaggtcgc
caaaaatctgaacgagagcctcatcgatcttcaggagctgggcaaatatgagcagtacatca
agtggccttggtatatttggcttggcttcatcgccggcctgatcgccatagtaatggtcaca
attatggccggcctctctttatggatgtgctccaatggatcgttacaatgcagaatttgcat
ttaa
PDI-Modified S protein with H5i Hemagglutinin CT (V3) DNA
(SEQ ID NO: 72)
atggcgaaaaacgttgcgattttcggcttattgttttctcttcttgtgttggttccttctca
gatcttcgcggtgaatcttacgacgcgaacacagttaccacccgcatatacaaatagcttca
ctcggggtgtttattaccccgacaaagtgttcaggtcctccgtgctccactcaacacaggac
ctctttcttcctttcttttctaacgtgacatggtttcatgccattcatgtatccggcactaa
cggtactaagaggttcgataatcctgtgctccctttcaatgacggcgtttactttgcaagca
cagagaagagtaacatcatccgaggttggatctttggcactaccctcgattcaaagacgcag
agcctcctcattgtgaacaatgccactaacgtggtgatcaaagtttgcgagtttcagttctg
caatgaccctttcttgggggtgtactatcataagaacaacaagtcttggatggaatctgaat
tccgcgtctatagcagcgccaacaactgcacctttgaatacgtgtcccagcccttccttatg
gacctggagggaaagcagggaaactttaagaatctgagagagttcgtgtttaaaaatatcga
cggctattttaagatctattctaagcacacgcctattaatctcgtgcgcgatcttccacaag
gcttcagcgccctggaaccactcgtggacctcccaattggtatcaacatcactagatttcag
actctgcttgccctccaccgatcctatctgacacccggagactcctctagcggctggactgc
cggcgctgccgcttattacgttggttatcttcagccacgcacgttcctgctgaagtataacg
agaatggtactattaccgatgccgtggattgtgcccttgaccccctgtccgaaactaagtgc
acactcaagtcattcactgtggaaaaaggaatctaccagacaagcaattttcgggtccagcc
tactgagagcattgtgcgctttcctaacatcacaaatctttgccccttcggagaggttttca
atgctacacggtttgcctccgtgtatgcctggaaccgcaagagaatttccaattgcgtggcc
gattactccgtgctctacaatagtgcaagctttagcacctttaagtgctatggcgtatcccc
tactaagcttaacgacttgtgtttcacaaacgtgtatgccgactcctttgtgatacggggcg
acgaagttagacagatagcaccaggacagacgggaaagatagctgactacaactataagctt
cctgatgacttcactggctgcgttatcgcgtggaattctaacaacctggactcaaaagtcgg
cggcaactataactatctctatcggctgttccgcaagagtaaccttaagccctttgagagag
atataagcactgaaatctaccaggctggcagtacgccctgtaatggcgtggaaggctttaat
tgttattttccactgcaatcctatggttttcagccaaccaatggcgtgggctaccaaccata
ccgcgtcgtggtgctctcctttgaactgctccacgctcccgcgactgtctgcggccccaaga
agtccacgaaccttgtgaagaataagtgcgttaattttaatttcaacggcctcactggaaca
ggagtgctcactgagagtaacaagaagttcctgccatttcaacaatttggcagagacatagc
cgatactactgacgccgttagggacccccagaccctcgagattctcgatataacgccctgct
ccttcggtggagtttccgtgatcacgccaggcaccaataccagtaaccaggtcgccgtgctg
tatcaggatgtcaactgtactgaggtgcccgtagccatccatgcggatcagctcacaccaac
ttggagggtgtacagcaccggctccaatgtattccagactcgggccggatgccttattggcg
ccgaacacgtgaacaatagttacgaatgcgatattccaattggcgccggaatctgtgctagc
taccagactcagacgaactccccaggcagcgccagcagcgttgccagccagtcaatcatcgc
ttatacaatgtcacttggagccgaaaactccgtggcttactcaaacaacagcatcgccatcc
ccacaaacttcaccatatccgtgacaactgagattctgccagtgtccatgactaagacgtcc
gtagattgcactatgtacatatgcggcgacagcacagaatgttctaatctgctgctgcaata
tggaagcttctgcactcaactgaacagagcgctcacaggcatcgccgtggagcaggataaga
atacccaggaggtgttcgcccaagttaagcagatctacaagaccccacccataaaggatttc
ggtggattcaattttagtcagatactcccagacccatctaagccatccaagaggagctttat
cgaggatcttttgtttaacaaagttactctggccgacgccggtttcatcaagcagtacggag
attgcctcggcgacatcgctgctcgtgacctcatctgtgcgcaaaagtttaacggtctgacg
gtgctgcctcccctccttactgatgaaatgatcgcccagtataccagcgcactcctcgctgg
caccataacatccggttggacattcggcgctggtgcagcactgcagataccattcgccatgc
aaatggcatatcgtttcaacggtatcggtgtcacacagaatgtcctatatgagaaccagaag
ctgatcgcaaatcagttcaatagtgccatcggaaaaatccaggatagccttagcagcacagc
ctcagcccttggcaaactccaggatgtcgtgaaccagaatgcccaggctctcaataccctcg
tgaagcagctctcatctaatttcggcgcaatttccagtgtcctcaacgacatcctcagccgc
ctcgacccccccgaggccgaagtgcagattgacagactgattacaggtcgactccagagcct
ccagacttacgtgactcagcagctgataagagccgccgagataagggccagcgctaacctgg
ctgccacaaagatgtctgagtgcgtgctgggccagtccaagagagtagacttctgtggcaaa
ggctaccatctgatgagcttcccacaatccgcacctcacggcgtagtgttcctccacgtgac
atatgtaccggctcaggagaagaatttcactaccgctcctgctatatgccatgatggaaagg
ctcacttcccccgggagggggtgttcgtgtccaacggcacccattggtttgtgactcagcgg
aatttctacgaaccccagatcataaccactgacaacacatttgtgtccggaaattgtgacgt
ggtcattggaatagtgaacaacactgtttatgatccactgcagccagaacttgacagcttta
aggaggagctcgacaagtacttcaagaatcatacgtcaccagatgtggacctcggagatatt
agcggtatcaatgccagtgttgtcaatattcagaaggaaatagaccgccttaatgaggtcgc
caaaaatctgaacgagagcctcatcgatcttcaggagctgggcaaatatgagcagtacatca
agtggccttggtatatttggcttggcttcatcgccggcctgatcgccatagtaatggtcaca
attatgctctgctgcatgtgctccaatggatcgttacaatgcagaatttgcatttaa
PDI-Modified S protein with H5i Hemagglutinin CT (V4) DNA
(SEQ ID NO: 73)
atggcgaaaaacgttgcgattttcggcttattgttttctcttcttgtgttggttccttctca
gatcttcgcggtgaatcttacgacgcgaacacagttaccacccgcatatacaaatagcttca
ctcggggtgtttattaccccgacaaagtgttcaggtcctccgtgctccactcaacacaggac
ctctttcttcctttcttttctaacgtgacatggtttcatgccattcatgtatccggcactaa
cggtactaagaggttcgataatcctgtgctccctttcaatgacggcgtttactttgcaagca
cagagaagagtaacatcatccgaggttggatctttggcactaccctcgattcaaagacgcag
agcctcctcattgtgaacaatgccactaacgtggtgatcaaagtttgcgagtttcagttctg
caatgaccctttcttgggggtgtactatcataagaacaacaagtcttggatggaatctgaat
tccgcgtctatagcagcgccaacaactgcacctttgaatacgtgtcccagcccttccttatg
gacctggagggaaagcagggaaactttaagaatctgagagagttcgtgtttaaaaatatcga
cggctattttaagatctattctaagcacacgcctattaatctcgtgcgcgatcttccacaag
gcttcagcgccctggaaccactcgtggacctcccaattggtatcaacatcactagatttcag
actctgcttgccctccaccgatcctatctgacacccggagactcctctagcggctggactgc
cggcgctgccgcttattacgttggttatcttcagccacgcacgttcctgctgaagtataacg
agaatggtactattaccgatgccgtggattgtgcccttgaccccctgtccgaaactaagtgc
acactcaagtcattcactgtggaaaaaggaatctaccagacaagcaattttcgggtccagcc
tactgagagcattgtgcgctttcctaacatcacaaatctttgccccttcggagaggttttca
atgctacacggtttgcctccgtgtatgcctggaaccgcaagagaatttccaattgcgtggcc
gattactccgtgctctacaatagtgcaagctttagcacctttaagtgctatggcgtatcccc
tactaagcttaacgacttgtgtttcacaaacgtgtatgccgactcctttgtgatacggggcg
acgaagttagacagatagcaccaggacagacgggaaagatagctgactacaactataagctt
cctgatgacttcactggctgcgttatcgcgtggaattctaacaacctggactcaaaagtcgg
cggcaactataactatctctatcggctgttccgcaagagtaaccttaagccctttgagagag
atataagcactgaaatctaccaggctggcagtacgccctgtaatggcgtggaaggctttaat
tgttattttccactgcaatcctatggttttcagccaaccaatggcgtgggctaccaaccata
ccgcgtcgtggtgctctcctttgaactgctccacgctcccgcgactgtctgcggccccaaga
agtccacgaaccttgtgaagaataagtgcgttaattttaatttcaacggcctcactggaaca
ggagtgctcactgagagtaacaagaagttcctgccatttcaacaatttggcagagacatagc
cgatactactgacgccgttagggacccccagaccctcgagattctcgatataacgccctgct
ccttcggtggagtttccgtgatcacgccaggcaccaataccagtaaccaggtcgccgtgctg
tatcaggatgtcaactgtactgaggtgcccgtagccatccatgcggatcagctcacaccaac
ttggagggtgtacagcaccggctccaatgtattccagactcgggccggatgccttattggcg
ccgaacacgtgaacaatagttacgaatgcgatattccaattggcgccggaatctgtgctagc
taccagactcagacgaactccccaggcagcgccagcagcgttgccagccagtcaatcatcgc
ttatacaatgtcacttggagccgaaaactccgtggcttactcaaacaacagcatcgccatcc
ccacaaacttcaccatatccgtgacaactgagattctgccagtgtccatgactaagacgtcc
gtagattgcactatgtacatatgcggcgacagcacagaatgttctaatctgctgctgcaata
tggaagcttctgcactcaactgaacagagcgctcacaggcatcgccgtggagcaggataaga
atacccaggaggtgttcgcccaagttaagcagatctacaagaccccacccataaaggatttc
ggtggattcaattttagtcagatactcccagacccatctaagccatccaagaggagctttat
cgaggatcttttgtttaacaaagttactctggccgacgccggtttcatcaagcagtacggag
attgcctcggcgacatcgctgctcgtgacctcatctgtgcgcaaaagtttaacggtctgacg
gtgctgcctcccctccttactgatgaaatgatcgcccagtataccagcgcactcctcgctgg
caccataacatccggttggacattcggcgctggtgcagcactgcagataccattcgccatgc
aaatggcatatcgtttcaacggtatcggtgtcacacagaatgtcctatatgagaaccagaag
ctgatcgcaaatcagttcaatagtgccatcggaaaaatccaggatagccttagcagcacagc
ctcagcccttggcaaactccaggatgtcgtgaaccagaatgcccaggctctcaataccctcg
tgaagcagctctcatctaatttcggcgcaatttccagtgtcctcaacgacatcctcagccgc
ctcgacccccccgaggccgaagtgcagattgacagactgattacaggtcgactccagagcct
ccagacttacgtgactcagcagctgataagagccgccgagataagggccagcgctaacctgg
ctgccacaaagatgtctgagtgcgtgctgggccagtccaagagagtagacttctgtggcaaa
ggctaccatctgatgagcttcccacaatccgcacctcacggcgtagtgttcctccacgtgac
atatgtaccggctcaggagaagaatttcactaccgctcctgctatatgccatgatggaaagg
ctcacttcccccggggggggtgcttcgtgtccaacggcacccattggtttgtgactcagcgg
aatttctacgaaccccagatcataaccactgacaacacatttgtgtccggaaattgtgacgt
ggtcattggaatagtgaacaacactgtttatgatccactgcagccagaacttgacagcttta
aggaggagctcgacaagtacttcaagaatcatacgtcaccagatgtggacctcggagatatt
agcggtatcaatgccagtgttgtcaatattcagaaggaaatagaccgccttaatgaggtcgc
caaaaatctgaacgagagcctcatcgatcttcaggagctgggcaaatatgagcagtacatca
agtggccttggtatatttggcttggcttcatcgccggcctgatcgccatagtaatggtcaca
attatgctctgctgctccaatggatcgttacaatgcagaatttgcatttaa
PDI-S-protein + H1 Cal DNA
(SEQ ID NO: 74)
atggcgaaaaacgttgcgattttcggcttattgttttctcttcttgtgttggttccttctca
gatcttcgcggtgaatcttacgacgcgaacacagttaccacccgcatatacaaatagcttca
ctcggggtgtttattaccccgacaaagtgttcaggtcctccgtgctccactcaacacaggac
ctctttcttcctttcttttctaacgtgacatggtttcatgccattcatgtatccggcactaa
cggtactaagaggttcgataatcctgtgctccctttcaatgacggcgtttactttgcaagca
cagagaagagtaacatcatccgaggttggatctttggcactaccctcgattcaaagacgcag
agcctcctcattgtgaacaatgccactaacgtggtgatcaaagtttgcgagtttcagttctg
caatgaccctttcttgggggtgtactatcataagaacaacaagtcttggatggaatctgaat
tccgcgtctatagcagcgccaacaactgcacctttgaatacgtgtcccagcccttccttatg
gacctggagggaaagcagggaaactttaagaatctgagagagttcgtgtttaaaaatatcga
cggctattttaagatctattctaagcacacgcctattaatctcgtgcgcgatcttccacaag
gcttcagcgccctggaaccactcgtggacctcccaattggtatcaacatcactagatttcag
actctgcttgccctccaccgatcctatctgacacccggagactcctctagcggctggactgc
cggcgctgccgcttattacgttggttatcttcagccacgcacgttcctgctgaagtataacg
agaatggtactattaccgatgccgtggattgtgcccttgaccccctgtccgaaactaagtgc
acactcaagtcattcactgtggaaaaaggaatctaccagacaagcaattttcgggtccagcc
tactgagagcattgtgcgctttcctaacatcacaaatctttgccccttcggagaggttttca
atgctacacggtttgcctccgtgtatgcctggaaccgcaagagaatttccaattgcgtggcc
gattactccgtgctctacaatagtgcaagctttagcacctttaagtgctatggcgtatcccc
tactaagcttaacgacttgtgtttcacaaacgtgtatgccgactcctttgtgatacggggcg
acgaagttagacagatagcaccaggacagacgggaaagatagctgactacaactataagctt
cctgatgacttcactggctgcgttatcgcgtggaattctaacaacctggactcaaaagtcgg
cggcaactataactatctctatcggctgttccgcaagagtaaccttaagccctttgagagag
atataagcactgaaatctaccaggctggcagtacgccctgtaatggcgtggaaggctttaat
tgttattttccactgcaatcctatggttttcagccaaccaatggcgtgggctaccaaccata
ccgcgtcgtggtgctctcctttgaactgctccacgctcccgcgactgtctgcggccccaaga
agtccacgaaccttgtgaagaataagtgcgttaattttaatttcaacggcctcactggaaca
ggagtgctcactgagagtaacaagaagttcctgccatttcaacaatttggcagagacatagc
cgatactactgacgccgttagggacccccagaccctcgagattctcgatataacgccctgct
ccttcggtggagtttccgtgatcacgccaggcaccaataccagtaaccaggtcgccgtgctg
tatcaggatgtcaactgtactgaggtgcccgtagccatccatgcggatcagctcacaccaac
ttggagggtgtacagcaccggctccaatgtattccagactcgggccggatgccttattggcg
ccgaacacgtgaacaatagttacgaatgcgatattccaattggcgccggaatctgtgctagc
taccagactcagacgaactccccaggcagcgccagcagcgttgccagccagtcaatcatcgc
ttatacaatgtcacttggagccgaaaactccgtggcttactcaaacaacagcatcgccatcc
ccacaaacttcaccatatccgtgacaactgagattctgccagtgtccatgactaagacgtcc
gtagattgcactatgtacatatgcggcgacagcacagaatgttctaatctgctgctgcaata
tggaagcttctgcactcaactgaacagagcgctcacaggcatcgccgtggagcaggataaga
atacccaggaggtgttcgcccaagttaagcagatctacaagaccccacccataaaggatttc
ggtggattcaattttagtcagatactcccagacccatctaagccatccaagaggagctttat
cgaggatcttttgtttaacaaagttactctggccgacgccggtttcatcaagcagtacggag
attgcctcggcgacatcgctgctcgtgacctcatctgtgcgcaaaagtttaacggtctgacg
gtgctgcctcccctccttactgatgaaatgatcgcccagtataccagcgcactcctcgctgg
caccataacatccggttggacattcggcgctggtgcagcactgcagataccattcgccatgc
aaatggcatatcgtttcaacggtatcggtgtcacacagaatgtcctatatgagaaccagaag
ctgatcgcaaatcagttcaatagtgccatcggaaaaatccaggatagccttagcagcacagc
ctcagcccttggcaaactccaggatgtcgtgaaccagaatgcccaggctctcaataccctcg
tgaagcagctctcatctaatttcggcgcaatttccagtgtcctcaacgacatcctcagccgc
ctcgacccccccgaggccgaagtgcagattgacagactgattacaggtcgactccagagcct
ccagacttacgtgactcagcagctgataagagccgccgagataagggccagcgctaacctgg
ctgccacaaagatgtctgagtgcgtgctgggccagtccaagagagtagacttctgtggcaaa
ggctaccatctgatgagcttcccacaatccgcacctcacggcgtagtgttcctccacgtgac
atatgtaccggctcaggagaagaatttcactaccgctcctgctatatgccatgatggaaagg
ctcacttcccccgggagggggtgttcgtgtccaacggcacccattggtttgtgactcagcgg
aatttctacgaaccccagatcataaccactgacaacacatttgtgtccggaaattgtgacgt
ggtcattggaatagtgaacaacactgtttatgatccactgcagccagaacttgacagcttta
aggaggagctcgacaagtacttcaagaatcatacgtcaccagatgtggacctcggagatatt
agcggtatcaatgccagtgttgtcaatattcagaaggaaatagaccgccttaatgaggtcgc
caaaaatctgaacgagagcctcatcgatcttcaggagctgggcaaatatgagcagtacatca
agtggccttggtatatttggcttggcttcatcgccggcctgatcgccatagtaatggtcaca
attatgctcagcttctggatgtgctctaatgggtctctacagtgtagaatatgtatttaa
PDI-S-protein + H3 Minn DNA
(SEQ ID NO: 75)
atggcgaaaaacgttgcgattttcggcttattgttttctcttcttgtgttggttccttctca
gatcttcgcggtgaatcttacgacgcgaacacagttaccacccgcatatacaaatagcttca
ctcggggtgtttattaccccgacaaagtgttcaggtcctccgtgctccactcaacacaggac
ctctttcttcctttcttttctaacgtgacatggtttcatgccattcatgtatccggcactaa
cggtactaagaggttcgataatcctgtgctccctttcaatgacggcgtttactttgcaagca
cagagaagagtaacatcatccgaggttggatctttggcactaccctcgattcaaagacgcag
agcctcctcattgtgaacaatgccactaacgtggtgatcaaagtttgcgagtttcagttctg
caatgaccctttcttgggggtgtactatcataagaacaacaagtcttggatggaatctgaat
tccgcgtctatagcagcgccaacaactgcacctttgaatacgtgtcccagcccttccttatg
gacctggagggaaagcagggaaactttaagaatctgagagagttcgtgtttaaaaatatcga
cggctattttaagatctattctaagcacacgcctattaatctcgtgcgcgatcttccacaag
gcttcagcgccctggaaccactcgtggacctcccaattggtatcaacatcactagatttcag
actctgcttgccctccaccgatcctatctgacacccggagactcctctagcggctggactgc
cggcgctgccgcttattacgttggttatcttcagccacgcacgttcctgctgaagtataacg
agaatggtactattaccgatgccgtggattgtgcccttgaccccctgtccgaaactaagtgc
acactcaagtcattcactgtggaaaaaggaatctaccagacaagcaattttcgggtccagcc
tactgagagcattgtgcgctttcctaacatcacaaatctttgccccttcggagaggttttca
atgctacacggtttgcctccgtgtatgcctggaaccgcaagagaatttccaattgcgtggcc
gattactccgtgctctacaatagtgcaagctttagcacctttaagtgctatggcgtatcccc
tactaagcttaacgacttgtgtttcacaaacgtgtatgccgactcctttgtgatacggggcg
acgaagttagacagatagcaccaggacagacgggaaagatagctgactacaactataagctt
cctgatgacttcactggctgcgttatcgcgtggaattctaacaacctggactcaaaagtcgg
cggcaactataactatctctatcggctgttccgcaagagtaaccttaagccctttgagagag
atataagcactgaaatctaccaggctggcagtacgccctgtaatggcgtggaaggctttaat
tgttattttccactgcaatcctatggttttcagccaaccaatggcgtgggctaccaaccata
ccgcgtcgtggtgctctcctttgaactgctccacgctcccgcgactgtctgcggccccaaga
agtccacgaaccttgtgaagaataagtgcgttaattttaatttcaacggcctcactggaaca
ggagtgctcactgagagtaacaagaagttcctgccatttcaacaatttggcagagacatagc
cgatactactgacgccgttagggacccccagaccctcgagattctcgatataacgccctgct
ccttcggtggagtttccgtgatcacgccaggcaccaataccagtaaccaggtcgccgtgctg
tatcaggatgtcaactgtactgaggtgcccgtagccatccatgcggatcagctcacaccaac
ttggagggtgtacagcaccggctccaatgtattccagactcgggccggatgccttattggcg
ccgaacacgtgaacaatagttacgaatgcgatattccaattggcgccggaatctgtgctagc
taccagactcagacgaactccccaggcagcgccagcagcgttgccagccagtcaatcatcgc
ttatacaatgtcacttggagccgaaaactccgtggcttactcaaacaacagcatcgccatcc
ccacaaacttcaccatatccgtgacaactgagattctgccagtgtccatgactaagacgtcc
gtagattgcactatgtacatatgcggcgacagcacagaatgttctaatctgctgctgcaata
tggaagcttctgcactcaactgaacagagcgctcacaggcatcgccgtggagcaggataaga
atacccaggaggtgttcgcccaagttaagcagatctacaagaccccacccataaaggatttc
ggtggattcaattttagtcagatactcccagacccatctaagccatccaagaggagctttat
cgaggatcttttgtttaacaaagttactctggccgacgccggtttcatcaagcagtacggag
attgcctcggcgacatcgctgctcgtgacctcatctgtgcgcaaaagtttaacggtctgacg
gtgctgcctcccctccttactgatgaaatgatcgcccagtataccagcgcactcctcgctgg
caccataacatccggttggacattcggcgctggtgcagcactgcagataccattcgccatgc
aaatggcatatcgtttcaacggtatcggtgtcacacagaatgtcctatatgagaaccagaag
ctgatcgcaaatcagttcaatagtgccatcggaaaaatccaggatagccttagcagcacagc
ctcagcccttggcaaactccaggatgtcgtgaaccagaatgcccaggctctcaataccctcg
tgaagcagctctcatctaatttcggcgcaatttccagtgtcctcaacgacatcctcagccgc
ctcgacccccccgaggccgaagtgcagattgacagactgattacaggtcgactccagagcct
ccagacttacgtgactcagcagctgataagagccgccgagataagggccagcgctaacctgg
ctgccacaaagatgtctgagtgcgtgctgggccagtccaagagagtagacttctgtggcaaa
ggctaccatctgatgagcttcccacaatccgcacctcacggcgtagtgttcctccacgtgac
atatgtaccggctcaggagaagaatttcactaccgctcctgctatatgccatgatggaaagg
ctcacttcccccgggagggggtgttcgtgtccaacggcacccattggtttgtgactcagcgg
aatttctacgaaccccagatcataaccactgacaacacatttgtgtccggaaattgtgacgt
ggtcattggaatagtgaacaacactgtttatgatccactgcagccagaacttgacagcttta
aggaggagctcgacaagtacttcaagaatcatacgtcaccagatgtggacctcggagatatt
agcggtatcaatgccagtgttgtcaatattcagaaggaaatagaccgccttaatgaggtcgc
caaaaatctgaacgagagcctcatcgatcttcaggagctgggcaaatatgagcagtacatca
agtggccttggtatatttggcttggcttcatcgccggcctgatcgccatagtaatggtcaca
attatgctcatgtgggcctgtcagaagggcaacatcagatgcaacatctgcatctaa
PDI-S-protein + H6 HK DNA
(SEQ ID NO: 76)
atggcgaaaaacgttgcgattttcggcttattgttttctcttcttgtgttggttccttctca
gatcttcgcggtgaatcttacgacgcgaacacagttaccacccgcatatacaaatagcttca
ctcggggtgtttattaccccgacaaagtgttcaggtcctccgtgctccactcaacacaggac
ctctttcttcctttcttttctaacgtgacatggtttcatgccattcatgtatccggcactaa
cggtactaagaggttcgataatcctgtgctccctttcaatgacggcgtttactttgcaagca
cagagaagagtaacatcatccgaggttggatctttggcactaccctcgattcaaagacgcag
agcctcctcattgtgaacaatgccactaacgtggtgatcaaagtttgcgagtttcagttctg
caatgaccctttcttgggggtgtactatcataagaacaacaagtcttggatggaatctgaat
tccgcgtctatagcagcgccaacaactgcacctttgaatacgtgtcccagcccttccttatg
gacctggagggaaagcagggaaactttaagaatctgagagagttcgtgtttaaaaatatcga
cggctattttaagatctattctaagcacacgcctattaatctcgtgcgcgatcttccacaag
gcttcagcgccctggaaccactcgtggacctcccaattggtatcaacatcactagatttcag
actctgcttgccctccaccgatcctatctgacacccggagactcctctagcggctggactgc
cggcgctgccgcttattacgttggttatcttcagccacgcacgttcctgctgaagtataacg
agaatggtactattaccgatgccgtggattgtgcccttgaccccctgtccgaaactaagtgc
acactcaagtcattcactgtggaaaaaggaatctaccagacaagcaattttcgggtccagcc
tactgagagcattgtgcgctttcctaacatcacaaatctttgccccttcggagaggttttca
atgctacacggtttgcctccgtgtatgcctggaaccgcaagagaatttccaattgcgtggcc
gattactccgtgctctacaatagtgcaagctttagcacctttaagtgctatggcgtatcccc
tactaagcttaacgacttgtgtttcacaaacgtgtatgccgactcctttgtgatacggggcg
acgaagttagacagatagcaccaggacagacgggaaagatagctgactacaactataagctt
cctgatgacttcactggctgcgttatcgcgtggaattctaacaacctggactcaaaagtcgg
cggcaactataactatctctatcggctgttccgcaagagtaaccttaagccctttgagagag
atataagcactgaaatctaccaggctggcagtacgccctgtaatggcgtggaaggctttaat
tgttattttccactgcaatcctatggttttcagccaaccaatggcgtgggctaccaaccata
ccgcgtcgtggtgctctcctttgaactgctccacgctcccgcgactgtctgcggccccaaga
agtccacgaaccttgtgaagaataagtgcgttaattttaatttcaacggcctcactggaaca
ggagtgctcactgagagtaacaagaagttcctgccatttcaacaatttggcagagacatagc
cgatactactgacgccgttagggacccccagaccctcgagattctcgatataacgccctgct
ccttcggtggagtttccgtgatcacgccaggcaccaataccagtaaccaggtcgccgtgctg
tatcaggatgtcaactgtactgaggtgcccgtagccatccatgcggatcagctcacaccaac
ttggagggtgtacagcaccggctccaatgtattccagactcgggccggatgccttattggcg
ccgaacacgtgaacaatagttacgaatgcgatattccaattggcgccggaatctgtgctagc
taccagactcagacgaactccccaggcagcgccagcagcgttgccagccagtcaatcatcgc
ttatacaatgtcacttggagccgaaaactccgtggcttactcaaacaacagcatcgccatcc
ccacaaacttcaccatatccgtgacaactgagattctgccagtgtccatgactaagacgtcc
gtagattgcactatgtacatatgcggcgacagcacagaatgttctaatctgctgctgcaata
tggaagcttctgcactcaactgaacagagcgctcacaggcatcgccgtggagcaggataaga
atacccaggaggtgttcgcccaagttaagcagatctacaagaccccacccataaaggatttc
ggtggattcaattttagtcagatactcccagacccatctaagccatccaagaggagctttat
cgaggatcttttgtttaacaaagttactctggccgacgccggtttcatcaagcagtacggag
attgcctcggcgacatcgctgctcgtgacctcatctgtgcgcaaaagtttaacggtctgacg
gtgctgcctcccctccttactgatgaaatgatcgcccagtataccagcgcactcctcgctgg
caccataacatccggttggacattcggcgctggtgcagcactgcagataccattcgccatgc
aaatggcatatcgtttcaacggtatcggtgtcacacagaatgtcctatatgagaaccagaag
ctgatcgcaaatcagttcaatagtgccatcggaaaaatccaggatagccttagcagcacagc
ctcagcccttggcaaactccaggatgtcgtgaaccagaatgcccaggctctcaataccctcg
tgaagcagctctcatctaatttcggcgcaatttccagtgtcctcaacgacatcctcagccgc
ctcgacccccccgaggccgaagtgcagattgacagactgattacaggtcgactccagagcct
ccagacttacgtgactcagcagctgataagagccgccgagataagggccagcgctaacctgg
ctgccacaaagatgtctgagtgcgtgctgggccagtccaagagagtagacttctgtggcaaa
ggctaccatctgatgagcttcccacaatccgcacctcacggcgtagtgttcctccacgtgac
atatgtaccggctcaggagaagaatttcactaccgctcctgctatatgccatgatggaaagg
ctcacttcccccgggagggggtgttcgtgtccaacggcacccattggtttgtgactcagcgg
aatttctacgaaccccagatcataaccactgacaacacatttgtgtccggaaattgtgacgt
ggtcattggaatagtgaacaacactgtttatgatccactgcagccagaacttgacagcttta
aggaggagctcgacaagtacttcaagaatcatacgtcaccagatgtggacctcggagatatt
agcggtatcaatgccagtgttgtcaatattcagaaggaaatagaccgccttaatgaggtcgc
caaaaatctgaacgagagcctcatcgatcttcaggagctgggcaaatatgagcagtacatca
agtggccttggtatatttggcttggcttcatcgccggcctgatcgccatagtaatggtcaca
attatgctcggtctttggatgtgttcaaatggttcaatgcagtgcaggatatgtatataa
PDI-S-protein + H7 Guangdong DNA
(SEQ ID NO: 77)
atggcgaaaaacgttgcgattttcggcttattgttttctcttcttgtgttggttccttctca
gatcttcgcggtgaatcttacgacgcgaacacagttaccacccgcatatacaaatagcttca
ctcggggtgtttattaccccgacaaagtgttcaggtcctccgtgctccactcaacacaggac
ctctttcttcctttcttttctaacgtgacatggtttcatgccattcatgtatccggcactaa
cggtactaagaggttcgataatcctgtgctccctttcaatgacggcgtttactttgcaagca
cagagaagagtaacatcatccgaggttggatctttggcactaccctcgattcaaagacgcag
agcctcctcattgtgaacaatgccactaacgtggtgatcaaagtttgcgagtttcagttctg
caatgaccctttcttgggggtgtactatcataagaacaacaagtcttggatggaatctgaat
tccgcgtctatagcagcgccaacaactgcacctttgaatacgtgtcccagcccttccttatg
gacctggagggaaagcagggaaactttaagaatctgagagagttcgtgtttaaaaatatcga
cggctattttaagatctattctaagcacacgcctattaatctcgtgcgcgatcttccacaag
gcttcagcgccctggaaccactcgtggacctcccaattggtatcaacatcactagatttcag
actctgcttgccctccaccgatcctatctgacacccggagactcctctagcggctggactgc
cggcgctgccgcttattacgttggttatcttcagccacgcacgttcctgctgaagtataacg
agaatggtactattaccgatgccgtggattgtgcccttgaccccctgtccgaaactaagtgc
acactcaagtcattcactgtggaaaaaggaatctaccagacaagcaattttcgggtccagcc
tactgagagcattgtgcgctttcctaacatcacaaatctttgccccttcggagaggttttca
atgctacacggtttgcctccgtgtatgcctggaaccgcaagagaatttccaattgcgtggcc
gattactccgtgctctacaatagtgcaagctttagcacctttaagtgctatggcgtatcccc
tactaagcttaacgacttgtgtttcacaaacgtgtatgccgactcctttgtgatacggggcg
acgaagttagacagatagcaccaggacagacgggaaagatagctgactacaactataagctt
cctgatgacttcactggctgcgttatcgcgtggaattctaacaacctggactcaaaagtcgg
cggcaactataactatctctatcggctgttccgcaagagtaaccttaagccctttgagagag
atataagcactgaaatctaccaggctggcagtacgccctgtaatggcgtggaaggctttaat
tgttattttccactgcaatcctatggttttcagccaaccaatggcgtgggctaccaaccata
ccgcgtcgtggtgctctcctttgaactgctccacgctcccgcgactgtctgcggccccaaga
agtccacgaaccttgtgaagaataagtgcgttaattttaatttcaacggcctcactggaaca
ggagtgctcactgagagtaacaagaagttcctgccatttcaacaatttggcagagacatagc
cgatactactgacgccgttagggacccccagaccctcgagattctcgatataacgccctgct
ccttcggtggagtttccgtgatcacgccaggcaccaataccagtaaccaggtcgccgtgctg
tatcaggatgtcaactgtactgaggtgcccgtagccatccatgcggatcagctcacaccaac
ttggagggtgtacagcaccggctccaatgtattccagactcgggccggatgccttattggcg
ccgaacacgtgaacaatagttacgaatgcgatattccaattggcgccggaatctgtgctagc
taccagactcagacgaactccccaggcagcgccagcagcgttgccagccagtcaatcatcgc
ttatacaatgtcacttggagccgaaaactccgtggcttactcaaacaacagcatcgccatcc
ccacaaacttcaccatatccgtgacaactgagattctgccagtgtccatgactaagacgtcc
gtagattgcactatgtacatatgcggcgacagcacagaatgttctaatctgctgctgcaata
tggaagcttctgcactcaactgaacagagcgctcacaggcatcgccgtggagcaggataaga
atacccaggaggtgttcgcccaagttaagcagatctacaagaccccacccataaaggatttc
ggtggattcaattttagtcagatactcccagacccatctaagccatccaagaggagctttat
cgaggatcttttgtttaacaaagttactctggccgacgccggtttcatcaagcagtacggag
attgcctcggcgacatcgctgctcgtgacctcatctgtgcgcaaaagtttaacggtctgacg
gtgctgcctcccctccttactgatgaaatgatcgcccagtataccagcgcactcctcgctgg
caccataacatccggttggacattcggcgctggtgcagcactgcagataccattcgccatgc
aaatggcatatcgtttcaacggtatcggtgtcacacagaatgtcctatatgagaaccagaag
ctgatcgcaaatcagttcaatagtgccatcggaaaaatccaggatagccttagcagcacagc
ctcagcccttggcaaactccaggatgtcgtgaaccagaatgcccaggctctcaataccctcg
tgaagcagctctcatctaatttcggcgcaatttccagtgtcctcaacgacatcctcagccgc
ctcgacccccccgaggccgaagtgcagattgacagactgattacaggtcgactccagagcct
ccagacttacgtgactcagcagctgataagagccgccgagataagggccagcgctaacctgg
ctgccacaaagatgtctgagtgcgtgctgggccagtccaagagagtagacttctgtggcaaa
ggctaccatctgatgagcttcccacaatccgcacctcacggcgtagtgttcctccacgtgac
atatgtaccggctcaggagaagaatttcactaccgctcctgctatatgccatgatggaaagg
ctcacttcccccgggagggggtgttcgtgtccaacggcacccattggtttgtgactcagcgg
aatttctacgaaccccagatcataaccactgacaacacatttgtgtccggaaattgtgacgt
ggtcattggaatagtgaacaacactgtttatgatccactgcagccagaacttgacagcttta
aggaggagctcgacaagtacttcaagaatcatacgtcaccagatgtggacctcggagatatt
agcggtatcaatgccagtgttgtcaatattcagaaggaaatagaccgccttaatgaggtcgc
caaaaatctgaacgagagcctcatcgatcttcaggagctgggcaaatatgagcagtacatca
agtggccttggtatatttggcttggcttcatcgccggcctgatcgccatagtaatggtcaca
attatgctcgtcttcatatgtgtgaagaatggaaacatgcggtgcactatttgtatataa
PDI-S-protein + H9 HK DNA
(SEQ ID NO: 78)
atggcgaaaaacgttgcgattttcggcttattgttttctcttcttgtgttggttccttctca
gatcttcgcggtgaatcttacgacgcgaacacagttaccacccgcatatacaaatagcttca
ctcggggtgtttattaccccgacaaagtgttcaggtcctccgtgctccactcaacacaggac
ctctttcttcctttcttttctaacgtgacatggtttcatgccattcatgtatccggcactaa
cggtactaagaggttcgataatcctgtgctccctttcaatgacggcgtttactttgcaagca
cagagaagagtaacatcatccgaggttggatctttggcactaccctcgattcaaagacgcag
agcctcctcattgtgaacaatgccactaacgtggtgatcaaagtttgcgagtttcagttctg
caatgaccctttcttgggggtgtactatcataagaacaacaagtcttggatggaatctgaat
tccgcgtctatagcagcgccaacaactgcacctttgaatacgtgtcccagcccttccttatg
gacctggagggaaagcagggaaactttaagaatctgagagagttcgtgtttaaaaatatcga
cggctattttaagatctattctaagcacacgcctattaatctcgtgcgcgatcttccacaag
gcttcagcgccctggaaccactcgtggacctcccaattggtatcaacatcactagatttcag
actctgcttgccctccaccgatcctatctgacacccggagactcctctagcggctggactgc
cggcgctgccgcttattacgttggttatcttcagccacgcacgttcctgctgaagtataacg
agaatggtactattaccgatgccgtggattgtgcccttgaccccctgtccgaaactaagtgc
acactcaagtcattcactgtggaaaaaggaatctaccagacaagcaattttcgggtccagcc
tactgagagcattgtgcgctttcctaacatcacaaatctttgccccttcggagaggttttca
atgctacacggtttgcctccgtgtatgcctggaaccgcaagagaatttccaattgcgtggcc
gattactccgtgctctacaatagtgcaagctttagcacctttaagtgctatggcgtatcccc
tactaagcttaacgacttgtgtttcacaaacgtgtatgccgactcctttgtgatacggggcg
acgaagttagacagatagcaccaggacagacgggaaagatagctgactacaactataagctt
cctgatgacttcactggctgcgttatcgcgtggaattctaacaacctggactcaaaagtcgg
cggcaactataactatctctatcggctgttccgcaagagtaaccttaagccctttgagagag
atataagcactgaaatctaccaggctggcagtacgccctgtaatggcgtggaaggctttaat
tgttattttccactgcaatcctatggttttcagccaaccaatggcgtgggctaccaaccata
ccgcgtcgtggtgctctcctttgaactgctccacgctcccgcgactgtctgcggccccaaga
agtccacgaaccttgtgaagaataagtgcgttaattttaatttcaacggcctcactggaaca
ggagtgctcactgagagtaacaagaagttcctgccatttcaacaatttggcagagacatagc
cgatactactgacgccgttagggacccccagaccctcgagattctcgatataacgccctgct
ccttcggtggagtttccgtgatcacgccaggcaccaataccagtaaccaggtcgccgtgctg
tatcaggatgtcaactgtactgaggtgcccgtagccatccatgcggatcagctcacaccaac
ttggagggtgtacagcaccggctccaatgtattccagactcgggccggatgccttattggcg
ccgaacacgtgaacaatagttacgaatgcgatattccaattggcgccggaatctgtgctagc
taccagactcagacgaactccccaggcagcgccagcagcgttgccagccagtcaatcatcgc
ttatacaatgtcacttggagccgaaaactccgtggcttactcaaacaacagcatcgccatcc
ccacaaacttcaccatatccgtgacaactgagattctgccagtgtccatgactaagacgtcc
gtagattgcactatgtacatatgcggcgacagcacagaatgttctaatctgctgctgcaata
tggaagcttctgcactcaactgaacagagcgctcacaggcatcgccgtggagcaggataaga
atacccaggaggtgttcgcccaagttaagcagatctacaagaccccacccataaaggatttc
ggtggattcaattttagtcagatactcccagacccatctaagccatccaagaggagctttat
cgaggatcttttgtttaacaaagttactctggccgacgccggtttcatcaagcagtacggag
attgcctcggcgacatcgctgctcgtgacctcatctgtgcgcaaaagtttaacggtctgacg
gtgctgcctcccctccttactgatgaaatgatcgcccagtataccagcgcactcctcgctgg
caccataacatccggttggacattcggcgctggtgcagcactgcagataccattcgccatgc
aaatggcatatcgtttcaacggtatcggtgtcacacagaatgtcctatatgagaaccagaag
ctgatcgcaaatcagttcaatagtgccatcggaaaaatccaggatagccttagcagcacagc
ctcagcccttggcaaactccaggatgtcgtgaaccagaatgcccaggctctcaataccctcg
tgaagcagctctcatctaatttcggcgcaatttccagtgtcctcaacgacatcctcagccgc
ctcgacccccccgaggccgaagtgcagattgacagactgattacaggtcgactccagagcct
ccagacttacgtgactcagcagctgataagagccgccgagataagggccagcgctaacctgg
ctgccacaaagatgtctgagtgcgtgctgggccagtccaagagagtagacttctgtggcaaa
ggctaccatctgatgagcttcccacaatccgcacctcacggcgtagtgttcctccacgtgac
atatgtaccggctcaggagaagaatttcactaccgctcctgctatatgccatgatggaaagg
ctcacttcccccgggagggggtgttcgtgtccaacggcacccattggtttgtgactcagcgg
aatttctacgaaccccagatcataaccactgacaacacatttgtgtccggaaattgtgacgt
ggtcattggaatagtgaacaacactgtttatgatccactgcagccagaacttgacagcttta
aggaggagctcgacaagtacttcaagaatcatacgtcaccagatgtggacctcggagatatt
agcggtatcaatgccagtgttgtcaatattcagaaggaaatagaccgccttaatgaggtcgc
caaaaatctgaacgagagcctcatcgatcttcaggagctgggcaaatatgagcagtacatca
agtggccttggtatatttggcttggcttcatcgccggcctgatcgccatagtaatggtcaca
attatgctcctgttctgggccatgtccaatggatcttgcagatgcaacatttgtatataa
PDI-S-protein + B/ Wash DNA
(SEQ ID NO: 79)
atggcgaaaaacgttgcgattttcggcttattgttttctcttcttgtgttggttccttctca
gatcttcgcggtgaatcttacgacgcgaacacagttaccacccgcatatacaaatagcttca
ctcggggtgtttattaccccgacaaagtgttcaggtcctccgtgctccactcaacacaggac
ctctttcttcctttcttttctaacgtgacatggtttcatgccattcatgtatccggcactaa
cggtactaagaggttcgataatcctgtgctccctttcaatgacggcgtttactttgcaagca
cagagaagagtaacatcatccgaggttggatctttggcactaccctcgattcaaagacgcag
agcctcctcattgtgaacaatgccactaacgtggtgatcaaagtttgcgagtttcagttctg
caatgaccctttcttgggggtgtactatcataagaacaacaagtcttggatggaatctgaat
tccgcgtctatagcagcgccaacaactgcacctttgaatacgtgtcccagcccttccttatg
gacctggagggaaagcagggaaactttaagaatctgagagagttcgtgtttaaaaatatcga
cggctattttaagatctattctaagcacacgcctattaatctcgtgcgcgatcttccacaag
gcttcagcgccctggaaccactcgtggacctcccaattggtatcaacatcactagatttcag
actctgcttgccctccaccgatcctatctgacacccggagactcctctagcggctggactgc
cggcgctgccgcttattacgttggttatcttcagccacgcacgttcctgctgaagtataacg
agaatggtactattaccgatgccgtggattgtgcccttgaccccctgtccgaaactaagtgc
acactcaagtcattcactgtggaaaaaggaatctaccagacaagcaattttcgggtccagcc
tactgagagcattgtgcgctttcctaacatcacaaatctttgccccttcggagaggttttca
atgctacacggtttgcctccgtgtatgcctggaaccgcaagagaatttccaattgcgtggcc
gattactccgtgctctacaatagtgcaagctttagcacctttaagtgctatggcgtatcccc
tactaagcttaacgacttgtgtttcacaaacgtgtatgccgactcctttgtgatacggggcg
acgaagttagacagatagcaccaggacagacgggaaagatagctgactacaactataagctt
cctgatgacttcactggctgcgttatcgcgtggaattctaacaacctggactcaaaagtcgg
cggcaactataactatctctatcggctgttccgcaagagtaaccttaagccctttgagagag
atataagcactgaaatctaccaggctggcagtacgccctgtaatggcgtggaaggctttaat
tgttattttccactgcaatcctatggttttcagccaaccaatggcgtgggctaccaaccata
ccgcgtcgtggtgctctcctttgaactgctccacgctcccgcgactgtctgcggccccaaga
agtccacgaaccttgtgaagaataagtgcgttaattttaatttcaacggcctcactggaaca
ggagtgctcactgagagtaacaagaagttcctgccatttcaacaatttggcagagacatagc
cgatactactgacgccgttagggacccccagaccctcgagattctcgatataacgccctgct
ccttcggtggagtttccgtgatcacgccaggcaccaataccagtaaccaggtcgccgtgctg
tatcaggatgtcaactgtactgaggtgcccgtagccatccatgcggatcagctcacaccaac
ttggagggtgtacagcaccggctccaatgtattccagactcgggccggatgccttattggcg
ccgaacacgtgaacaatagttacgaatgcgatattccaattggcgccggaatctgtgctagc
taccagactcagacgaactccccaggcagcgccagcagcgttgccagccagtcaatcatcgc
ttatacaatgtcacttggagccgaaaactccgtggcttactcaaacaacagcatcgccatcc
ccacaaacttcaccatatccgtgacaactgagattctgccagtgtccatgactaagacgtcc
gtagattgcactatgtacatatgcggcgacagcacagaatgttctaatctgctgctgcaata
tggaagcttctgcactcaactgaacagagcgctcacaggcatcgccgtggagcaggataaga
atacccaggaggtgttcgcccaagttaagcagatctacaagaccccacccataaaggatttc
ggtggattcaattttagtcagatactcccagacccatctaagccatccaagaggagctttat
cgaggatcttttgtttaacaaagttactctggccgacgccggtttcatcaagcagtacggag
attgcctcggcgacatcgctgctcgtgacctcatctgtgcgcaaaagtttaacggtctgacg
gtgctgcctcccctccttactgatgaaatgatcgcccagtataccagcgcactcctcgctgg
caccataacatccggttggacattcggcgctggtgcagcactgcagataccattcgccatgc
aaatggcatatcgtttcaacggtatcggtgtcacacagaatgtcctatatgagaaccagaag
ctgatcgcaaatcagttcaatagtgccatcggaaaaatccaggatagccttagcagcacagc
ctcagcccttggcaaactccaggatgtcgtgaaccagaatgcccaggctctcaataccctcg
tgaagcagctctcatctaatttcggcgcaatttccagtgtcctcaacgacatcctcagccgc
ctcgacccccccgaggccgaagtgcagattgacagactgattacaggtcgactccagagcct
ccagacttacgtgactcagcagctgataagagccgccgagataagggccagcgctaacctgg
ctgccacaaagatgtctgagtgcgtgctgggccagtccaagagagtagacttctgtggcaaa
ggctaccatctgatgagcttcccacaatccgcacctcacggcgtagtgttcctccacgtgac
atatgtaccggctcaggagaagaatttcactaccgctcctgctatatgccatgatggaaagg
ctcacttcccccgggagggggtgttcgtgtccaacggcacccattggtttgtgactcagcgg
aatttctacgaaccccagatcataaccactgacaacacatttgtgtccggaaattgtgacgt
ggtcattggaatagtgaacaacactgtttatgatccactgcagccagaacttgacagcttta
aggaggagctcgacaagtacttcaagaatcatacgtcaccagatgtggacctcggagatatt
agcggtatcaatgccagtgttgtcaatattcagaaggaaatagaccgccttaatgaggtcgc
caaaaatctgaacgagagcctcatcgatcttcaggagctgggcaaatatgagcagtacatca
agtggccttggtatatttggcttggcttcatcgccggcctgatcgccatagtaatggtcaca
attatgctcgttgtttatatggtctccagagacaatgtttcttgctccatttgtctataa
IF-H1HawaiiCT.r
(SEQ ID NO: 80)
acgacacgactaaggcctttaaatacatattctacactgtagagaccc
IF-H3MinnesotaCT.r
(SEQ ID NO: 81)
acgacacgactaaggcctttagatgcagatgttgcatctgatgttgcccttctg
IF-HongKongCT.r
(SEQ ID NO: 82)
acgacacgactaaggcctttatatacatatcctgcactgcattgaaccattt
IF-GuangdongCT.r
(SEQ ID NO: 83)
acgacacgactaaggcctttatatacaaatagtgcaccgcatgtttcca
IF-H9HKCT.r
(SEQ ID NO: 84)
acgacacgactaaggcctttatatacaaatgttgcatctgcaagatccat
IF-BWashCT.r
(SEQ ID NO: 85)
acgacacgactaaggcctttatagacaaatggagcaagaaacattgtctc
IF(nbHEL40)-PDI.c
(SEQ ID NO: 86)
ccaaaacacattgagcaaaatggcgaaaaacgttgcgattttcggcttat
IF(AvB + wtCT).r
(SEQ ID NO: 87)
ACGACACGACTAAGGCCTTTAGGTATAATGGAGTTTCACCCCCTTCAGAA
PDI-SARS-COV-1 wtTMCT-DNA
(SEQ ID NO: 88)
atggcgaaaaacgttgcgattttcggcttattgttttctcttcttgtgttggttccttctca
gatcttcgcgTCCGATCTGGATCGGTGCACTACATTTGACGATGTGCAGGCACCTAATTATA
CTCAGCACACCTCTTCCATGCGGGGCGTGTACTACCCCGACGAGATTTTTAGAAGTGACACA
CTGTACCTGACCCAAGACCTTTTTCTCCCATTTTATAGCAATGTCACGGGATTCCACACTAT
CAATCACACATTCGGAAACCCTGTTATCCCCTTTAAAGACGGTATCTACTTTGCTGCTACTG
AGAAATCGAATGTTGTTCGCGGTTGGGTGTTCGGCTCAACCATGAACAACAAGAGTCAGTCA
GTAATAATTATAAACAACTCAACCAATGTCGTCATCAGGGCTTGCAACTTCGAGCTCTGCGA
TAACCCCTTCTTTGCGGTGTCCAAACCGATGGGCACTCAGACCCATACCATGATTTTTGACA
ATGCCTTTAATTGTACTTTCGAATACATCAGCGATGCCTTCTCGCTGGATGTGTCCGAGAAG
AGCGGTAACTTCAAGCATTTGCGGGAGTTCGTTTTCAAAAACAAAGACGGGTTTTTGTATGT
CTACAAAGGCTATCAGCCCATTGATGTGGTCAGGGATCTGCCCAGTGGTTTCAACACACTGA
AGCCCATCTTCAAGTTGCCACTCGGCATCAACATCACTAATTTCCGCGCCATCCTAACTGCT
TTTTCGCCAGCGCAGGATATTTGGGGGACATCCGCCGCAGCTTACTTCGTGGGATATCTGAA
GCCCACCACTTTTATGCTAAAGTACGACGAGAATGGCACCATCACCGATGCCGTGGACTGCT
CACAGAATCCTCTCGCAGAGCTCAAGTGTTCAGTGAAGTCATTTGAGATCGACAAAGGGATC
TACCAAACATCTAACTTTCGCGTCGTGCCTTCCGGTGACGTAGTCCGTTTCCCAAATATCAC
TAACTTGTGCCCTTTTGGTGAAGTATTCAACGCAACCAAATTCCCCAGTGTGTATGCGTGGG
AGCGCAAAAAGATCAGTAACTGTGTGGCTGACTATAGTGTTCTGTACAATAGCACCTTCTTC
AGCACCTTCAAGTGTTATGGAGTGAGCGCTACAAAACTGAACGATCTTTGTTTCTCAAACGT
GTACGCCGATTCATTTGTCGTTAAAGGTGACGATGTGAGGCAGATCGCTCCAGGCCAGACAG
GTGTGATTGCTGACTATAATTACAAACTGCCAGACGACTTCATGGGGTGCGTGCTAGCTTGG
AATACAAGAAACATTGACGCCACCTCCACGGGAAATTACAATTACAAGTATCGTTACCTTCG
CCATGGAAAGTTGAGACCCTTCGAGCGTGATATAAGTAACGTGCCCTTTAGTCCAGATGGAA
AACCCTGCACACCCCCTGCTCTCAATTGCTATTGGCCTCTCAATGACTACGGCTTTTACACA
ACTACTGGCATCGGATACCAGCCTTACCGGGTCGTGGTGCTCAGTTTTGAGTTGCTTAACGC
ACCCGCCACCGTGTGTGGTCCTAAACTTTCTACTGACCTGATTAAAAACCAATGCGTCAACT
TCAATTTTAACGGGCTGACCGGCACCGGTGTCCTGACCCCTAGCTCTAAGAGATTCCAGCCT
TTTCAGCAGTTCGGGAGGGATGTGAGCGACTTTACCGACTCTGTCAGGGATCCAAAGACCAG
CGAGATACTGGATATCTCGCCCTGCAGTTTCGGTGGCGTGTCCGTTATTACACCTGGCACCA
ACGCCTCCTCAGAGGTGGCGGTGCTCTATCAAGATGTCAACTGCACTGATGTGTCAACTGCC
ATCCATGCCGATCAGCTGACCCCCGCCTGGCGCATCTACAGTACCGGGAACAACGTTTTTCA
GACCCAGGCCGGCTGTCTAATCGGCGCAGAGCACGTTGACACATCCTACGAATGTGACATAC
CTATCGGGGCAGGCATTTGCGCTAGCTACCATACCGTGTCACTGTTGGCTTCCACGTCACAA
AAGTCAATCGTTGCCTACACGATGAGTCTGGGGGCTGACTCATCTATCGCCTACAGCAACAA
TACCATTGCAATTCCCACAAACTTCAGTATCTCCATCACAACAGAGGTGATGCCCGTTTCTA
TGGCTAAAACATCAGTCGATTGCAATATGTATATATGCGGCGATAGTACTGAGTGCGCCAAT
CTCTTGTTACAGTACGGCTCCTTTTGTACCCAGCTGAACCGAGCACTGTCTGGAATCGCCGC
AGAACAGGATCGCAATACCCGGGAAGTCTTCGCCCAGGTGAAGCAGATGTACAAAACGCCCA
CTCTCAAGTATTTCGGCGGATTCAACTTTTCTCAGATTTTGCCTGACCCGCTCAAGCCAACA
AAACGATCTTTTATCGAAGACCTTCTGTTTAACAAGGTCACACTGGCGGATGCTGGGTTCAT
GAAACAGTACGGTGAATGCCTGGGGGACATCAATGCCAGAGATCTGATCTGCGCCCAGAAAT
TCAATGGCTTAACAGTCCTCCCACCTCTCTTGACCGACGATATGATCGCTGCGTACACCGCT
GCTCTGGTATCGGGCACCGCGACTGCTGGCTGGACCTTTGGTGCCGGAGCCGCACTCCAGAT
CCCATTCGCCATGCAGATGGCCTACCGCTTCAACGGAATCGGGGTCACCCAGAACGTGCTGT
ATGAGAACCAGAAACAGATCGCCAATCAGTTCAATAAGGCAATTAGTCAGATTCAGGAGAGT
CTTACCACTACCAGCACCGCCCTGGGCAAGCTGCAAGATGTTGTGAACCAGAATGCGCAGGC
ATTAAACACTCTGGTTAAACAGCTGAGCTCAAATTTTGGTGCAATCTCTTCAGTTCTGAACG
ATATCCTGAGTCGGCTGGATCCGCCAGAGGCTGAAGTGCAAATTGATCGTTTGATCACCGGG
AGGCTACAATCTCTGCAGACGTACGTGACCCAGCAGCTCATCCGGGCAGCCGAAATTCGCGC
ATCAGCCAACCTCGCTGCAACTAAGATGTCTGAGTGCGTGCTGGGCCAGAGTAAGAGGGTGG
ACTTTTGTGGTAAGGGATACCACCTCATGTCCTTTCCGCAAGCGGCTCCCCACGGCGTGGTT
TTCTTACACGTTACCTATGTGCCATCCCAAGAACGCAATTTCACCACCGCTCCAGCTATCTG
TCATGAGGGCAAAGCATATTTCCCCAGGGAAGGAGTATTTGTGTTTAATGGCACGTCCTGGT
TTATAACCCAACGTAACTTTTTCTCCCCACAGATTATCACAACCGACAACACATTCGTGTCT
GGGAATTGTGACGTCGTGATCGGGATCATTAACAATACCGTTTACGATCCCTTGCAGCCCGA
GCTTGACTCCTTTAAAGAGGAACTAGACAAATACTTTAAGAATCACACCTCACCGGACGTAG
ATTTGGGAGACATCTCTGGAATTAATGCCTCTGTGGTGAATATCCAGAAGGAGATCGACCGC
CTGAATGAAGTCGCCAAGAACCTCAACGAGTCCCTGATAGATCTGCAAGAACTGGGCAAATA
TGAACAGTACATCAAATGGCCGTGGTACGTGTGGTTGGGCTTTATCGCTGGACTTATTGCAA
TCGTGATGGTGACGATTCTGCTCTGCTGTATGACTTCCTGCTGCTCTTGTCTGAAGGGCGCC
TGTAGCTGTGGTTCCTGCTGCAAGTTCGACGAAGACGACTCCGAACCAGTTCTGAAGGGGGT
GAAACTCCATTATACCTAA
PDI-SARS-COV-1 H5iTMCT-DNA
(SEQ ID NO: 89)
atggcgaaaaacgttgcgattttcggcttattgttttctcttcttgtgttggttccttctca
gatcttcgcgTCCGATCTGGATCGGTGCACTACATTTGACGATGTGCAGGCACCTAATTATA
CTCAGCACACCTCTTCCATGCGGGGCGTGTACTACCCCGACGAGATTTTTAGAAGTGACACA
CTGTACCTGACCCAAGACCTTTTTCTCCCATTTTATAGCAATGTCACGGGATTCCACACTAT
CAATCACACATTCGGAAACCCTGTTATCCCCTTTAAAGACGGTATCTACTTTGCTGCTACTG
AGAAATCGAATGTTGTTCGCGGTTGGGTGTTCGGCTCAACCATGAACAACAAGAGTCAGTCA
GTAATAATTATAAACAACTCAACCAATGTCGTCATCAGGGCTTGCAACTTCGAGCTCTGCGA
TAACCCCTTCTTTGCGGTGTCCAAACCGATGGGCACTCAGACCCATACCATGATTTTTGACA
ATGCCTTTAATTGTACTTTCGAATACATCAGCGATGCCTTCTCGCTGGATGTGTCCGAGAAG
AGCGGTAACTTCAAGCATTTGCGGGAGTTCGTTTTCAAAAACAAAGACGGGTTTTTGTATGT
CTACAAAGGCTATCAGCCCATTGATGTGGTCAGGGATCTGCCCAGTGGTTTCAACACACTGA
AGCCCATCTTCAAGTTGCCACTCGGCATCAACATCACTAATTTCCGCGCCATCCTAACTGCT
TTTTCGCCAGCGCAGGATATTTGGGGGACATCCGCCGCAGCTTACTTCGTGGGATATCTGAA
GCCCACCACTTTTATGCTAAAGTACGACGAGAATGGCACCATCACCGATGCCGTGGACTGCT
CACAGAATCCTCTCGCAGAGCTCAAGTGTTCAGTGAAGTCATTTGAGATCGACAAAGGGATC
TACCAAACATCTAACTTTCGCGTCGTGCCTTCCGGTGACGTAGTCCGTTTCCCAAATATCAC
TAACTTGTGCCCTTTTGGTGAAGTATTCAACGCAACCAAATTCCCCAGTGTGTATGCGTGGG
AGCGCAAAAAGATCAGTAACTGTGTGGCTGACTATAGTGTTCTGTACAATAGCACCTTCTTC
AGCACCTTCAAGTGTTATGGAGTGAGCGCTACAAAACTGAACGATCTTTGTTTCTCAAACGT
GTACGCCGATTCATTTGTCGTTAAAGGTGACGATGTGAGGCAGATCGCTCCAGGCCAGACAG
GTGTGATTGCTGACTATAATTACAAACTGCCAGACGACTTCATGGGGTGCGTGCTAGCTTGG
AATACAAGAAACATTGACGCCACCTCCACGGGAAATTACAATTACAAGTATCGTTACCTTCG
CCATGGAAAGTTGAGACCCTTCGAGCGTGATATAAGTAACGTGCCCTTTAGTCCAGATGGAA
AACCCTGCACACCCCCTGCTCTCAATTGCTATTGGCCTCTCAATGACTACGGCTTTTACACA
ACTACTGGCATCGGATACCAGCCTTACCGGGTCGTGGTGCTCAGTTTTGAGTTGCTTAACGC
ACCCGCCACCGTGTGTGGTCCTAAACTTTCTACTGACCTGATTAAAAACCAATGCGTCAACT
TCAATTTTAACGGGCTGACCGGCACCGGTGTCCTGACCCCTAGCTCTAAGAGATTCCAGCCT
TTTCAGCAGTTCGGGAGGGATGTGAGCGACTTTACCGACTCTGTCAGGGATCCAAAGACCAG
CGAGATACTGGATATCTCGCCCTGCAGTTTCGGTGGCGTGTCCGTTATTACACCTGGCACCA
ACGCCTCCTCAGAGGTGGCGGTGCTCTATCAAGATGTCAACTGCACTGATGTGTCAACTGCC
ATCCATGCCGATCAGCTGACCCCCGCCTGGCGCATCTACAGTACCGGGAACAACGTTTTTCA
GACCCAGGCCGGCTGTCTAATCGGCGCAGAGCACGTTGACACATCCTACGAATGTGACATAC
CTATCGGGGCAGGCATTTGCGCTAGCTACCATACCGTGTCACTGTTGGCTTCCACGTCACAA
AAGTCAATCGTTGCCTACACGATGAGTCTGGGGGCTGACTCATCTATCGCCTACAGCAACAA
TACCATTGCAATTCCCACAAACTTCAGTATCTCCATCACAACAGAGGTGATGCCCGTTTCTA
TGGCTAAAACATCAGTCGATTGCAATATGTATATATGCGGCGATAGTACTGAGTGCGCCAAT
CTCTTGTTACAGTACGGCTCCTTTTGTACCCAGCTGAACCGAGCACTGTCTGGAATCGCCGC
AGAACAGGATCGCAATACCCGGGAAGTCTTCGCCCAGGTGAAGCAGATGTACAAAACGCCCA
CTCTCAAGTATTTCGGCGGATTCAACTTTTCTCAGATTTTGCCTGACCCGCTCAAGCCAACA
AAACGATCTTTTATCGAAGACCTTCTGTTTAACAAGGTCACACTGGCGGATGCTGGGTTCAT
GAAACAGTACGGTGAATGCCTGGGGGACATCAATGCCAGAGATCTGATCTGCGCCCAGAAAT
TCAATGGCTTAACAGTCCTCCCACCTCTCTTGACCGACGATATGATCGCTGCGTACACCGCT
GCTCTGGTATCGGGCACCGCGACTGCTGGCTGGACCTTTGGTGCCGGAGCCGCACTCCAGAT
CCCATTCGCCATGCAGATGGCCTACCGCTTCAACGGAATCGGGGTCACCCAGAACGTGCTGT
ATGAGAACCAGAAACAGATCGCCAATCAGTTCAATAAGGCAATTAGTCAGATTCAGGAGAGT
CTTACCACTACCAGCACCGCCCTGGGCAAGCTGCAAGATGTTGTGAACCAGAATGCGCAGGC
ATTAAACACTCTGGTTAAACAGCTGAGCTCAAATTTTGGTGCAATCTCTTCAGTTCTGAACG
ATATCCTGAGTCGGCTGGATCCGCCAGAGGCTGAAGTGCAAATTGATCGTTTGATCACCGGG
AGGCTACAATCTCTGCAGACGTACGTGACCCAGCAGCTCATCCGGGCAGCCGAAATTCGCGC
ATCAGCCAACCTCGCTGCAACTAAGATGTCTGAGTGCGTGCTGGGCCAGAGTAAGAGGGTGG
ACTTTTGTGGTAAGGGATACCACCTCATGTCCTTTCCGCAAGCGGCTCCCCACGGCGTGGTT
TTCTTACACGTTACCTATGTGCCATCCCAAGAACGCAATTTCACCACCGCTCCAGCTATCTG
TCATGAGGGCAAAGCATATTTCCCCAGGGAAGGAGTATTTGTGTTTAATGGCACGTCCTGGT
TTATAACCCAACGTAACTTTTTCTCCCCACAGATTATCACAACCGACAACACATTCGTGTCT
GGGAATTGTGACGTCGTGATCGGGATCATTAACAATACCGTTTACGATCCCTTGCAGCCCGA
GCTTGACTCCTTTAAAGAGGAACTAGACAAATACTTTAAGAATCACACCTCACCGGACGTAG
ATTTGGGAGACATCTCTGGAATTAATGCCTCTGTGGTGAATATCCAGAAGGAGATCGACCGC
CTGAATGAAGTCGCCAAGAACCTCAACGAGTCCCTGATAGATCTGCAAGAACTGGGCAAATA
TGAACAGTACATCAAATGGCCGTGGTACcaaatactgtcaatttattcaacagtggcgagtt
ccctagcactggcaatcatgatggctggtctatctttatggatgtgctccaatggatcgtta
caatgcagaatttgcattTAA
PDI-SARS-COV-1 H5iCT-DNA
(SEQ ID NO: 90)
atggcgaaaaacgttgcgattttcggcttattgttttctcttcttgtgttggttccttctca
gatcttcgcgTCCGATCTGGATCGGTGCACTACATTTGACGATGTGCAGGCACCTAATTATA
CTCAGCACACCTCTTCCATGCGGGGCGTGTACTACCCCGACGAGATTTTTAGAAGTGACACA
CTGTACCTGACCCAAGACCTTTTTCTCCCATTTTATAGCAATGTCACGGGATTCCACACTAT
CAATCACACATTCGGAAACCCTGTTATCCCCTTTAAAGACGGTATCTACTTTGCTGCTACTG
AGAAATCGAATGTTGTTCGCGGTTGGGTGTTCGGCTCAACCATGAACAACAAGAGTCAGTCA
GTAATAATTATAAACAACTCAACCAATGTCGTCATCAGGGCTTGCAACTTCGAGCTCTGCGA
TAACCCCTTCTTTGCGGTGTCCAAACCGATGGGCACTCAGACCCATACCATGATTTTTGACA
ATGCCTTTAATTGTACTTTCGAATACATCAGCGATGCCTTCTCGCTGGATGTGTCCGAGAAG
AGCGGTAACTTCAAGCATTTGCGGGAGTTCGTTTTCAAAAACAAAGACGGGTTTTTGTATGT
CTACAAAGGCTATCAGCCCATTGATGTGGTCAGGGATCTGCCCAGTGGTTTCAACACACTGA
AGCCCATCTTCAAGTTGCCACTCGGCATCAACATCACTAATTTCCGCGCCATCCTAACTGCT
TTTTCGCCAGCGCAGGATATTTGGGGGACATCCGCCGCAGCTTACTTCGTGGGATATCTGAA
GCCCACCACTTTTATGCTAAAGTACGACGAGAATGGCACCATCACCGATGCCGTGGACTGCT
CACAGAATCCTCTCGCAGAGCTCAAGTGTTCAGTGAAGTCATTTGAGATCGACAAAGGGATC
TACCAAACATCTAACTTTCGCGTCGTGCCTTCCGGTGACGTAGTCCGTTTCCCAAATATCAC
TAACTTGTGCCCTTTTGGTGAAGTATTCAACGCAACCAAATTCCCCAGTGTGTATGCGTGGG
AGCGCAAAAAGATCAGTAACTGTGTGGCTGACTATAGTGTTCTGTACAATAGCACCTTCTTC
AGCACCTTCAAGTGTTATGGAGTGAGCGCTACAAAACTGAACGATCTTTGTTTCTCAAACGT
GTACGCCGATTCATTTGTCGTTAAAGGTGACGATGTGAGGCAGATCGCTCCAGGCCAGACAG
GTGTGATTGCTGACTATAATTACAAACTGCCAGACGACTTCATGGGGTGCGTGCTAGCTTGG
AATACAAGAAACATTGACGCCACCTCCACGGGAAATTACAATTACAAGTATCGTTACCTTCG
CCATGGAAAGTTGAGACCCTTCGAGCGTGATATAAGTAACGTGCCCTTTAGTCCAGATGGAA
AACCCTGCACACCCCCTGCTCTCAATTGCTATTGGCCTCTCAATGACTACGGCTTTTACACA
ACTACTGGCATCGGATACCAGCCTTACCGGGTCGTGGTGCTCAGTTTTGAGTTGCTTAACGC
ACCCGCCACCGTGTGTGGTCCTAAACTTTCTACTGACCTGATTAAAAACCAATGCGTCAACT
TCAATTTTAACGGGCTGACCGGCACCGGTGTCCTGACCCCTAGCTCTAAGAGATTCCAGCCT
TTTCAGCAGTTCGGGAGGGATGTGAGCGACTTTACCGACTCTGTCAGGGATCCAAAGACCAG
CGAGATACTGGATATCTCGCCCTGCAGTTTCGGTGGCGTGTCCGTTATTACACCTGGCACCA
ACGCCTCCTCAGAGGTGGCGGTGCTCTATCAAGATGTCAACTGCACTGATGTGTCAACTGCC
ATCCATGCCGATCAGCTGACCCCCGCCTGGCGCATCTACAGTACCGGGAACAACGTTTTTCA
GACCCAGGCCGGCTGTCTAATCGGCGCAGAGCACGTTGACACATCCTACGAATGTGACATAC
CTATCGGGGCAGGCATTTGCGCTAGCTACCATACCGTGTCACTGTTGGCTTCCACGTCACAA
AAGTCAATCGTTGCCTACACGATGAGTCTGGGGGCTGACTCATCTATCGCCTACAGCAACAA
TACCATTGCAATTCCCACAAACTTCAGTATCTCCATCACAACAGAGGTGATGCCCGTTTCTA
TGGCTAAAACATCAGTCGATTGCAATATGTATATATGCGGCGATAGTACTGAGTGCGCCAAT
CTCTTGTTACAGTACGGCTCCTTTTGTACCCAGCTGAACCGAGCACTGTCTGGAATCGCCGC
AGAACAGGATCGCAATACCCGGGAAGTCTTCGCCCAGGTGAAGCAGATGTACAAAACGCCCA
CTCTCAAGTATTTCGGCGGATTCAACTTTTCTCAGATTTTGCCTGACCCGCTCAAGCCAACA
AAACGATCTTTTATCGAAGACCTTCTGTTTAACAAGGTCACACTGGCGGATGCTGGGTTCAT
GAAACAGTACGGTGAATGCCTGGGGGACATCAATGCCAGAGATCTGATCTGCGCCCAGAAAT
TCAATGGCTTAACAGTCCTCCCACCTCTCTTGACCGACGATATGATCGCTGCGTACACCGCT
GCTCTGGTATCGGGCACCGCGACTGCTGGCTGGACCTTTGGTGCCGGAGCCGCACTCCAGAT
CCCATTCGCCATGCAGATGGCCTACCGCTTCAACGGAATCGGGGTCACCCAGAACGTGCTGT
ATGAGAACCAGAAACAGATCGCCAATCAGTTCAATAAGGCAATTAGTCAGATTCAGGAGAGT
CTTACCACTACCAGCACCGCCCTGGGCAAGCTGCAAGATGTTGTGAACCAGAATGCGCAGGC
ATTAAACACTCTGGTTAAACAGCTGAGCTCAAATTTTGGTGCAATCTCTTCAGTTCTGAACG
ATATCCTGAGTCGGCTGGATCCGCCAGAGGCTGAAGTGCAAATTGATCGTTTGATCACCGGG
AGGCTACAATCTCTGCAGACGTACGTGACCCAGCAGCTCATCCGGGCAGCCGAAATTCGCGC
ATCAGCCAACCTCGCTGCAACTAAGATGTCTGAGTGCGTGCTGGGCCAGAGTAAGAGGGTGG
ACTTTTGTGGTAAGGGATACCACCTCATGTCCTTTCCGCAAGCGGCTCCCCACGGCGTGGTT
TTCTTACACGTTACCTATGTGCCATCCCAAGAACGCAATTTCACCACCGCTCCAGCTATCTG
TCATGAGGGCAAAGCATATTTCCCCAGGGAAGGAGTATTTGTGTTTAATGGCACGTCCTGGT
TTATAACCCAACGTAACTTTTTCTCCCCACAGATTATCACAACCGACAACACATTCGTGTCT
GGGAATTGTGACGTCGTGATCGGGATCATTAACAATACCGTTTACGATCCCTTGCAGCCCGA
GCTTGACTCCTTTAAAGAGGAACTAGACAAATACTTTAAGAATCACACCTCACCGGACGTAG
ATTTGGGAGACATCTCTGGAATTAATGCCTCTGTGGTGAATATCCAGAAGGAGATCGACCGC
CTGAATGAAGTCGCCAAGAACCTCAACGAGTCCCTGATAGATCTGCAAGAACTGGGCAAATA
TGAACAGTACATCAAATGGCCGTGGTACGTGTGGTTGGGCTTTATCGCTGGACTTATTGCAA
TCGTGATGGTGACGATTCTGCTCtctttatggatgtgctccaatggatcgttacaatgcaga
atttgcattTAA
PDI-SARS-COV-1 H5iCT(V4)-DNA
(SEQ ID NO: 91)
atggcgaaaaacgttgcgattttcggcttattgttttctcttcttgtgttggttccttctca
gatcttcgcgTCCGATCTGGATCGGTGCACTACATTTGACGATGTGCAGGCACCTAATTATA
CTCAGCACACCTCTTCCATGCGGGGCGTGTACTACCCCGACGAGATTTTTAGAAGTGACACA
CTGTACCTGACCCAAGACCTTTTTCTCCCATTTTATAGCAATGTCACGGGATTCCACACTAT
CAATCACACATTCGGAAACCCTGTTATCCCCTTTAAAGACGGTATCTACTTTGCTGCTACTG
AGAAATCGAATGTTGTTCGCGGTTGGGTGTTCGGCTCAACCATGAACAACAAGAGTCAGTCA
GTAATAATTATAAACAACTCAACCAATGTCGTCATCAGGGCTTGCAACTTCGAGCTCTGCGA
TAACCCCTTCTTTGCGGTGTCCAAACCGATGGGCACTCAGACCCATACCATGATTTTTGACA
ATGCCTTTAATTGTACTTTCGAATACATCAGCGATGCCTTCTCGCTGGATGTGTCCGAGAAG
AGCGGTAACTTCAAGCATTTGCGGGAGTTCGTTTTCAAAAACAAAGACGGGTTTTTGTATGT
CTACAAAGGCTATCAGCCCATTGATGTGGTCAGGGATCTGCCCAGTGGTTTCAACACACTGA
AGCCCATCTTCAAGTTGCCACTCGGCATCAACATCACTAATTTCCGCGCCATCCTAACTGCT
TTTTCGCCAGCGCAGGATATTTGGGGGACATCCGCCGCAGCTTACTTCGTGGGATATCTGAA
GCCCACCACTTTTATGCTAAAGTACGACGAGAATGGCACCATCACCGATGCCGTGGACTGCT
CACAGAATCCTCTCGCAGAGCTCAAGTGTTCAGTGAAGTCATTTGAGATCGACAAAGGGATC
TACCAAACATCTAACTTTCGCGTCGTGCCTTCCGGTGACGTAGTCCGTTTCCCAAATATCAC
TAACTTGTGCCCTTTTGGTGAAGTATTCAACGCAACCAAATTCCCCAGTGTGTATGCGTGGG
AGCGCAAAAAGATCAGTAACTGTGTGGCTGACTATAGTGTTCTGTACAATAGCACCTTCTTC
AGCACCTTCAAGTGTTATGGAGTGAGCGCTACAAAACTGAACGATCTTTGTTTCTCAAACGT
GTACGCCGATTCATTTGTCGTTAAAGGTGACGATGTGAGGCAGATCGCTCCAGGCCAGACAG
GTGTGATTGCTGACTATAATTACAAACTGCCAGACGACTTCATGGGGTGCGTGCTAGCTTGG
AATACAAGAAACATTGACGCCACCTCCACGGGAAATTACAATTACAAGTATCGTTACCTTCG
CCATGGAAAGTTGAGACCCTTCGAGCGTGATATAAGTAACGTGCCCTTTAGTCCAGATGGAA
AACCCTGCACACCCCCTGCTCTCAATTGCTATTGGCCTCTCAATGACTACGGCTTTTACACA
ACTACTGGCATCGGATACCAGCCTTACCGGGTCGTGGTGCTCAGTTTTGAGTTGCTTAACGC
ACCCGCCACCGTGTGTGGTCCTAAACTTTCTACTGACCTGATTAAAAACCAATGCGTCAACT
TCAATTTTAACGGGCTGACCGGCACCGGTGTCCTGACCCCTAGCTCTAAGAGATTCCAGCCT
TTTCAGCAGTTCGGGAGGGATGTGAGCGACTTTACCGACTCTGTCAGGGATCCAAAGACCAG
CGAGATACTGGATATCTCGCCCTGCAGTTTCGGTGGCGTGTCCGTTATTACACCTGGCACCA
ACGCCTCCTCAGAGGTGGCGGTGCTCTATCAAGATGTCAACTGCACTGATGTGTCAACTGCC
ATCCATGCCGATCAGCTGACCCCCGCCTGGCGCATCTACAGTACCGGGAACAACGTTTTTCA
GACCCAGGCCGGCTGTCTAATCGGCGCAGAGCACGTTGACACATCCTACGAATGTGACATAC
CTATCGGGGCAGGCATTTGCGCTAGCTACCATACCGTGTCACTGTTGGCTTCCACGTCACAA
AAGTCAATCGTTGCCTACACGATGAGTCTGGGGGCTGACTCATCTATCGCCTACAGCAACAA
TACCATTGCAATTCCCACAAACTTCAGTATCTCCATCACAACAGAGGTGATGCCCGTTTCTA
TGGCTAAAACATCAGTCGATTGCAATATGTATATATGCGGCGATAGTACTGAGTGCGCCAAT
CTCTTGTTACAGTACGGCTCCTTTTGTACCCAGCTGAACCGAGCACTGTCTGGAATCGCCGC
AGAACAGGATCGCAATACCCGGGAAGTCTTCGCCCAGGTGAAGCAGATGTACAAAACGCCCA
CTCTCAAGTATTTCGGCGGATTCAACTTTTCTCAGATTTTGCCTGACCCGCTCAAGCCAACA
AAACGATCTTTTATCGAAGACCTTCTGTTTAACAAGGTCACACTGGCGGATGCTGGGTTCAT
GAAACAGTACGGTGAATGCCTGGGGGACATCAATGCCAGAGATCTGATCTGCGCCCAGAAAT
TCAATGGCTTAACAGTCCTCCCACCTCTCTTGACCGACGATATGATCGCTGCGTACACCGCT
GCTCTGGTATCGGGCACCGCGACTGCTGGCTGGACCTTTGGTGCCGGAGCCGCACTCCAGAT
CCCATTCGCCATGCAGATGGCCTACCGCTTCAACGGAATCGGGGTCACCCAGAACGTGCTGT
ATGAGAACCAGAAACAGATCGCCAATCAGTTCAATAAGGCAATTAGTCAGATTCAGGAGAGT
CTTACCACTACCAGCACCGCCCTGGGCAAGCTGCAAGATGTTGTGAACCAGAATGCGCAGGC
ATTAAACACTCTGGTTAAACAGCTGAGCTCAAATTTTGGTGCAATCTCTTCAGTTCTGAACG
ATATCCTGAGTCGGCTGGATCCGCCAGAGGCTGAAGTGCAAATTGATCGTTTGATCACCGGG
AGGCTACAATCTCTGCAGACGTACGTGACCCAGCAGCTCATCCGGGCAGCCGAAATTCGCGC
ATCAGCCAACCTCGCTGCAACTAAGATGTCTGAGTGCGTGCTGGGCCAGAGTAAGAGGGTGG
ACTTTTGTGGTAAGGGATACCACCTCATGTCCTTTCCGCAAGCGGCTCCCCACGGCGTGGTT
TTCTTACACGTTACCTATGTGCCATCCCAAGAACGCAATTTCACCACCGCTCCAGCTATCTG
TCATGAGGGCAAAGCATATTTCCCCAGGGAAGGAGTATTTGTGTTTAATGGCACGTCCTGGT
TTATAACCCAACGTAACTTTTTCTCCCCACAGATTATCACAACCGACAACACATTCGTGTCT
GGGAATTGTGACGTCGTGATCGGGATCATTAACAATACCGTTTACGATCCCTTGCAGCCCGA
GCTTGACTCCTTTAAAGAGGAACTAGACAAATACTTTAAGAATCACACCTCACCGGACGTAG
ATTTGGGAGACATCTCTGGAATTAATGCCTCTGTGGTGAATATCCAGAAGGAGATCGACCGC
CTGAATGAAGTCGCCAAGAACCTCAACGAGTCCCTGATAGATCTGCAAGAACTGGGCAAATA
TGAACAGTACATCAAATGGCCGTGGTACGTGTGGTTGGGCTTTATCGCTGGACTTATTGCAA
TCGTGATGGTGACGATTCTGCTCtgctgctccaatggatcgttacaatgcagaatttgcatt
TAA
PDI-SARS-COV-1 H1cCT-DNA
(SEQ ID NO: 92)
atggcgaaaaacgttgcgattttcggcttattgttttctcttcttgtgttggttccttctca
gatcttcgcgTCCGATCTGGATCGGTGCACTACATTTGACGATGTGCAGGCACCTAATTATA
CTCAGCACACCTCTTCCATGCGGGGCGTGTACTACCCCGACGAGATTTTTAGAAGTGACACA
CTGTACCTGACCCAAGACCTTTTTCTCCCATTTTATAGCAATGTCACGGGATTCCACACTAT
CAATCACACATTCGGAAACCCTGTTATCCCCTTTAAAGACGGTATCTACTTTGCTGCTACTG
AGAAATCGAATGTTGTTCGCGGTTGGGTGTTCGGCTCAACCATGAACAACAAGAGTCAGTCA
GTAATAATTATAAACAACTCAACCAATGTCGTCATCAGGGCTTGCAACTTCGAGCTCTGCGA
TAACCCCTTCTTTGCGGTGTCCAAACCGATGGGCACTCAGACCCATACCATGATTTTTGACA
ATGCCTTTAATTGTACTTTCGAATACATCAGCGATGCCTTCTCGCTGGATGTGTCCGAGAAG
AGCGGTAACTTCAAGCATTTGCGGGAGTTCGTTTTCAAAAACAAAGACGGGTTTTTGTATGT
CTACAAAGGCTATCAGCCCATTGATGTGGTCAGGGATCTGCCCAGTGGTTTCAACACACTGA
AGCCCATCTTCAAGTTGCCACTCGGCATCAACATCACTAATTTCCGCGCCATCCTAACTGCT
TTTTCGCCAGCGCAGGATATTTGGGGGACATCCGCCGCAGCTTACTTCGTGGGATATCTGAA
GCCCACCACTTTTATGCTAAAGTACGACGAGAATGGCACCATCACCGATGCCGTGGACTGCT
CACAGAATCCTCTCGCAGAGCTCAAGTGTTCAGTGAAGTCATTTGAGATCGACAAAGGGATC
TACCAAACATCTAACTTTCGCGTCGTGCCTTCCGGTGACGTAGTCCGTTTCCCAAATATCAC
TAACTTGTGCCCTTTTGGTGAAGTATTCAACGCAACCAAATTCCCCAGTGTGTATGCGTGGG
AGCGCAAAAAGATCAGTAACTGTGTGGCTGACTATAGTGTTCTGTACAATAGCACCTTCTTC
AGCACCTTCAAGTGTTATGGAGTGAGCGCTACAAAACTGAACGATCTTTGTTTCTCAAACGT
GTACGCCGATTCATTTGTCGTTAAAGGTGACGATGTGAGGCAGATCGCTCCAGGCCAGACAG
GTGTGATTGCTGACTATAATTACAAACTGCCAGACGACTTCATGGGGTGCGTGCTAGCTTGG
AATACAAGAAACATTGACGCCACCTCCACGGGAAATTACAATTACAAGTATCGTTACCTTCG
CCATGGAAAGTTGAGACCCTTCGAGCGTGATATAAGTAACGTGCCCTTTAGTCCAGATGGAA
AACCCTGCACACCCCCTGCTCTCAATTGCTATTGGCCTCTCAATGACTACGGCTTTTACACA
ACTACTGGCATCGGATACCAGCCTTACCGGGTCGTGGTGCTCAGTTTTGAGTTGCTTAACGC
ACCCGCCACCGTGTGTGGTCCTAAACTTTCTACTGACCTGATTAAAAACCAATGCGTCAACT
TCAATTTTAACGGGCTGACCGGCACCGGTGTCCTGACCCCTAGCTCTAAGAGATTCCAGCCT
TTTCAGCAGTTCGGGAGGGATGTGAGCGACTTTACCGACTCTGTCAGGGATCCAAAGACCAG
CGAGATACTGGATATCTCGCCCTGCAGTTTCGGTGGCGTGTCCGTTATTACACCTGGCACCA
ACGCCTCCTCAGAGGTGGCGGTGCTCTATCAAGATGTCAACTGCACTGATGTGTCAACTGCC
ATCCATGCCGATCAGCTGACCCCCGCCTGGCGCATCTACAGTACCGGGAACAACGTTTTTCA
GACCCAGGCCGGCTGTCTAATCGGCGCAGAGCACGTTGACACATCCTACGAATGTGACATAC
CTATCGGGGCAGGCATTTGCGCTAGCTACCATACCGTGTCACTGTTGGCTTCCACGTCACAA
AAGTCAATCGTTGCCTACACGATGAGTCTGGGGGCTGACTCATCTATCGCCTACAGCAACAA
TACCATTGCAATTCCCACAAACTTCAGTATCTCCATCACAACAGAGGTGATGCCCGTTTCTA
TGGCTAAAACATCAGTCGATTGCAATATGTATATATGCGGCGATAGTACTGAGTGCGCCAAT
CTCTTGTTACAGTACGGCTCCTTTTGTACCCAGCTGAACCGAGCACTGTCTGGAATCGCCGC
AGAACAGGATCGCAATACCCGGGAAGTCTTCGCCCAGGTGAAGCAGATGTACAAAACGCCCA
CTCTCAAGTATTTCGGCGGATTCAACTTTTCTCAGATTTTGCCTGACCCGCTCAAGCCAACA
AAACGATCTTTTATCGAAGACCTTCTGTTTAACAAGGTCACACTGGCGGATGCTGGGTTCAT
GAAACAGTACGGTGAATGCCTGGGGGACATCAATGCCAGAGATCTGATCTGCGCCCAGAAAT
TCAATGGCTTAACAGTCCTCCCACCTCTCTTGACCGACGATATGATCGCTGCGTACACCGCT
GCTCTGGTATCGGGCACCGCGACTGCTGGCTGGACCTTTGGTGCCGGAGCCGCACTCCAGAT
CCCATTCGCCATGCAGATGGCCTACCGCTTCAACGGAATCGGGGTCACCCAGAACGTGCTGT
ATGAGAACCAGAAACAGATCGCCAATCAGTTCAATAAGGCAATTAGTCAGATTCAGGAGAGT
CTTACCACTACCAGCACCGCCCTGGGCAAGCTGCAAGATGTTGTGAACCAGAATGCGCAGGC
ATTAAACACTCTGGTTAAACAGCTGAGCTCAAATTTTGGTGCAATCTCTTCAGTTCTGAACG
ATATCCTGAGTCGGCTGGATCCGCCAGAGGCTGAAGTGCAAATTGATCGTTTGATCACCGGG
AGGCTACAATCTCTGCAGACGTACGTGACCCAGCAGCTCATCCGGGCAGCCGAAATTCGCGC
ATCAGCCAACCTCGCTGCAACTAAGATGTCTGAGTGCGTGCTGGGCCAGAGTAAGAGGGTGG
ACTTTTGTGGTAAGGGATACCACCTCATGTCCTTTCCGCAAGCGGCTCCCCACGGCGTGGTT
TTCTTACACGTTACCTATGTGCCATCCCAAGAACGCAATTTCACCACCGCTCCAGCTATCTG
TCATGAGGGCAAAGCATATTTCCCCAGGGAAGGAGTATTTGTGTTTAATGGCACGTCCTGGT
TTATAACCCAACGTAACTTTTTCTCCCCACAGATTATCACAACCGACAACACATTCGTGTCT
GGGAATTGTGACGTCGTGATCGGGATCATTAACAATACCGTTTACGATCCCTTGCAGCCCGA
GCTTGACTCCTTTAAAGAGGAACTAGACAAATACTTTAAGAATCACACCTCACCGGACGTAG
ATTTGGGAGACATCTCTGGAATTAATGCCTCTGTGGTGAATATCCAGAAGGAGATCGACCGC
CTGAATGAAGTCGCCAAGAACCTCAACGAGTCCCTGATAGATCTGCAAGAACTGGGCAAATA
TGAACAGTACATCAAATGGCCGTGGTACGTGTGGTTGGGCTTTATCGCTGGACTTATTGCAA
TCGTGATGGTGACGATTCTGCTCagcttctggatgtgctctaatgggtctctacagtgtaga
atatgtattTAA
PDI-SARS-COV-1 wtTMCT-AA
(SEQ ID NO: 93)
MAKNVAIFGLLFSLLVLVPSQIFASDLDRCTTFDDVQAPNYTQHTSSMRGVYYPDEIFRSDT
LYLTQDLFLPFYSNVTGFHTINHTFGNPVIPFKDGIYFAATEKSNVVRGWVFGSTMNNKSQS
VIIINNSTNVVIRACNFELCDNPFFAVSKPMGTQTHTMIFDNAFNCTFEYISDAFSLDVSEK
SGNFKHLREFVFKNKDGFLYVYKGYQPIDVVRDLPSGFNTLKPIFKLPLGINITNFRAILTA
FSPAQDIWGTSAAAYFVGYLKPTTFMLKYDENGTITDAVDCSQNPLAELKCSVKSFEIDKGI
YQTSNFRVVPSGDVVRFPNITNLCPFGEVFNATKFPSVYAWERKKISNCVADYSVLYNSTFF
STFKCYGVSATKLNDLCFSNVYADSFVVKGDDVRQIAPGQTGVIADYNYKLPDDFMGCVLAW
NTRNIDATSTGNYNYKYRYLRHGKLRPFERDISNVPFSPDGKPCTPPALNCYWPLNDYGFYT
TTGIGYQPYRVVVLSFELLNAPATVCGPKLSTDLIKNQCVNFNFNGLTGTGVLTPSSKRFQP
FQQFGRDVSDFTDSVRDPKTSEILDISPCSFGGVSVITPGTNASSEVAVLYQDVNCTDVSTA
IHADQLTPAWRIYSTGNNVFQTQAGCLIGAEHVDTSYECDIPIGAGICASYHTVSLLASTSQ
KSIVAYTMSLGADSSIAYSNNTIAIPTNFSISITTEVMPVSMAKTSVDCNMYICGDSTECAN
LLLQYGSFCTQLNRALSGIAAEQDRNTREVFAQVKQMYKTPTLKYFGGFNFSQILPDPLKPT
KRSFIEDLLFNKVTLADAGFMKQYGECLGDINARDLICAQKFNGLTVLPPLLTDDMIAAYTA
ALVSGTATAGWTFGAGAALQIPFAMQMAYRFNGIGVTQNVLYENQKQIANQFNKAISQIQES
LTTTSTALGKLQDVVNQNAQALNTLVKQLSSNFGAISSVLNDILSRLDPPEAEVQIDRLITG
RLQSLQTYVTQQLIRAAEIRASANLAATKMSECVLGQSKRVDFCGKGYHLMSFPQAAPHGVV
FLHVTYVPSQERNFTTAPAICHEGKAYFPREGVFVFNGTSWFITQRNFFSPQIITTDNTFVS
GNCDVVIGIINNTVYDPLQPELDSFKEELDKYFKNHTSPDVDLGDISGINASVVNIQKEIDR
LNEVAKNLNESLIDLQELGKYEQYIKWPWYVWLGFIAGLIAIVMVTILLCCMTSCCSCLKGA
CSCGSCCKFDEDDSEPVLKGVKLHYT
PDI-SARS-COV-1 H5iTMCT-AA
(SEQ ID NO: 94)
MAKNVAIFGLLFSLLVLVPSQIFASDLDRCTTFDDVQAPNYTQHTSSMRGVYYPDEIFRSDT
LYLTQDLFLPFYSNVTGFHTINHTFGNPVIPFKDGIYFAATEKSNVVRGWVFGSTMNNKSQS
VIIINNSTNVVIRACNFELCDNPFFAVSKPMGTQTHTMIFDNAFNCTFEYISDAFSLDVSEK
SGNFKHLREFVFKNKDGFLYVYKGYQPIDVVRDLPSGFNTLKPIFKLPLGINITNFRAILTA
FSPAQDIWGTSAAAYFVGYLKPTTFMLKYDENGTITDAVDCSQNPLAELKCSVKSFEIDKGI
YQTSNFRVVPSGDVVRFPNITNLCPFGEVFNATKFPSVYAWERKKISNCVADYSVLYNSTFF
STFKCYGVSATKLNDLCFSNVYADSFVVKGDDVRQIAPGQTGVIADYNYKLPDDFMGCVLAW
NTRNIDATSTGNYNYKYRYLRHGKLRPFERDISNVPFSPDGKPCTPPALNCYWPLNDYGFYT
TTGIGYQPYRVVVLSFELLNAPATVCGPKLSTDLIKNQCVNFNFNGLTGTGVLTPSSKRFQP
FQQFGRDVSDFTDSVRDPKTSEILDISPCSFGGVSVITPGTNASSEVAVLYQDVNCTDVSTA
IHADQLTPAWRIYSTGNNVFQTQAGCLIGAEHVDTSYECDIPIGAGICASYHTVSLLASTSQ
KSIVAYTMSLGADSSIAYSNNTIAIPTNFSISITTEVMPVSMAKTSVDCNMYICGDSTECAN
LLLQYGSFCTQLNRALSGIAAEQDRNTREVFAQVKQMYKTPTLKYFGGFNFSQILPDPLKPT
KRSFIEDLLFNKVTLADAGFMKQYGECLGDINARDLICAQKFNGLTVLPPLLTDDMIAAYTA
ALVSGTATAGWTFGAGAALQIPFAMQMAYRFNGIGVTQNVLYENQKQIANQFNKAISQIQES
LTTTSTALGKLQDVVNQNAQALNTLVKQLSSNFGAISSVLNDILSRLDPPEAEVQIDRLITG
RLQSLQTYVTQQLIRAAEIRASANLAATKMSECVLGQSKRVDFCGKGYHLMSFPQAAPHGVV
FLHVTYVPSQERNFTTAPAICHEGKAYFPREGVFVFNGTSWFITQRNFFSPQIITTDNTFVS
GNCDVVIGIINNTVYDPLQPELDSFKEELDKYFKNHTSPDVDLGDISGINASVVNIQKEIDR
LNEVAKNLNESLIDLQELGKYEQYIKWPWYQILSIYSTVASSLALAIMMAGLSLWMCSNGSL
QCRICI
PDI-SARS-COV-1 H5iCT-AA
(SEQ ID NO: 95)
MAKNVAIFGLLFSLLVLVPSQIFASDLDRCTTFDDVQAPNYTQHTSSMRGVYYPDEIFRSDT
LYLTQDLFLPFYSNVTGFHTINHTFGNPVIPFKDGIYFAATEKSNVVRGWVFGSTMNNKSQS
VIIINNSTNVVIRACNFELCDNPFFAVSKPMGTQTHTMIFDNAFNCTFEYISDAFSLDVSEK
SGNFKHLREFVFKNKDGFLYVYKGYQPIDVVRDLPSGFNTLKPIFKLPLGINITNFRAILTA
FSPAQDIWGTSAAAYFVGYLKPTTFMLKYDENGTITDAVDCSQNPLAELKCSVKSFEIDKGI
YQTSNFRVVPSGDVVRFPNITNLCPFGEVFNATKFPSVYAWERKKISNCVADYSVLYNSTFF
STFKCYGVSATKLNDLCFSNVYADSFVVKGDDVRQIAPGQTGVIADYNYKLPDDFMGCVLAW
NTRNIDATSTGNYNYKYRYLRHGKLRPFERDISNVPFSPDGKPCTPPALNCYWPLNDYGFYT
TTGIGYQPYRVVVLSFELLNAPATVCGPKLSTDLIKNQCVNFNFNGLTGTGVLTPSSKRFQP
FQQFGRDVSDFTDSVRDPKTSEILDISPCSFGGVSVITPGTNASSEVAVLYQDVNCTDVSTA
IHADQLTPAWRIYSTGNNVFQTQAGCLIGAEHVDTSYECDIPIGAGICASYHTVSLLASTSQ
KSIVAYTMSLGADSSIAYSNNTIAIPTNFSISITTEVMPVSMAKTSVDCNMYICGDSTECAN
LLLQYGSFCTQLNRALSGIAAEQDRNTREVFAQVKQMYKTPTLKYFGGFNFSQILPDPLKPT
KRSFIEDLLENKVTLADAGFMKQYGECLGDINARDLICAQKFNGLTVLPPLLTDDMIAAYTA
ALVSGTATAGWTFGAGAALQIPFAMQMAYRFNGIGVTQNVLYENQKQIANQFNKAISQIQES
LTTTSTALGKLQDVVNQNAQALNTLVKQLSSNFGAISSVLNDILSRLDPPEAEVQIDRLITG
RLQSLQTYVTQQLIRAAEIRASANLAATKMSECVLGQSKRVDFCGKGYHLMSFPQAAPHGVV
FLHVTYVPSQERNFTTAPAICHEGKAYFPREGVFVFNGTSWFITQRNFFSPQIITTDNTFVS
GNCDVVIGIINNTVYDPLQPELDSFKEELDKYFKNHTSPDVDLGDISGINASVVNIQKEIDR
LNEVAKNLNESLIDLQELGKYEQYIKWPWYVWLGFIAGLIAIVMVTILLSLWMCSNGSLQCR
ICI
PDI-SARS-COV-1 H5iCT(V4)-AA
(SEQ ID NO: 96)
MAKNVAIFGLLFSLLVLVPSQIFASDLDRCTTFDDVQAPNYTQHTSSMRGVYYPDEIFRSDT
LYLTQDLFLPFYSNVTGFHTINHTFGNPVIPFKDGIYFAATEKSNVVRGWVFGSTMNNKSQS
VIIINNSTNVVIRACNFELCDNPFFAVSKPMGTQTHTMIFDNAFNCTFEYISDAFSLDVSEK
SGNFKHLREFVFKNKDGFLYVYKGYQPIDVVRDLPSGFNTLKPIFKLPLGINITNFRAILTA
FSPAQDIWGTSAAAYFVGYLKPTTFMLKYDENGTITDAVDCSQNPLAELKCSVKSFEIDKGI
YQTSNFRVVPSGDVVRFPNITNLCPFGEVFNATKFPSVYAWERKKISNCVADYSVLYNSTFF
STFKCYGVSATKLNDLCFSNVYADSFVVKGDDVRQIAPGQTGVIADYNYKLPDDFMGCVLAW
NTRNIDATSTGNYNYKYRYLRHGKLRPFERDISNVPFSPDGKPCTPPALNCYWPLNDYGFYT
TTGIGYQPYRVVVLSFELLNAPATVCGPKLSTDLIKNQCVNFNFNGLTGTGVLTPSSKRFQP
FQQFGRDVSDFTDSVRDPKTSEILDISPCSFGGVSVITPGTNASSEVAVLYQDVNCTDVSTA
IHADQLTPAWRIYSTGNNVFQTQAGCLIGAEHVDTSYECDIPIGAGICASYHTVSLLASTSQ
KSIVAYTMSLGADSSIAYSNNTIAIPTNFSISITTEVMPVSMAKTSVDCNMYICGDSTECAN
LLLQYGSFCTQLNRALSGIAAEQDRNTREVFAQVKQMYKTPTLKYFGGFNFSQILPDPLKPT
KRSFIEDLLFNKVTLADAGFMKQYGECLGDINARDLICAQKFNGLTVLPPLLTDDMIAAYTA
ALVSGTATAGWTFGAGAALQIPFAMQMAYRFNGIGVTQNVLYENQKQIANQFNKAISQIQES
LTTTSTALGKLQDVVNQNAQALNTLVKQLSSNFGAISSVLNDILSRLDPPEAEVQIDRLITG
RLQSLQTYVTQQLIRAAEIRASANLAATKMSECVLGQSKRVDFCGKGYHLMSFPQAAPHGVV
FLHVTYVPSQERNFTTAPAICHEGKAYFPREGVFVFNGTSWFITQRNFFSPQIITTDNTFVS
GNCDVVIGIINNTVYDPLQPELDSFKEELDKYFKNHTSPDVDLGDISGINASVVNIQKEIDR
LNEVAKNLNESLIDLQELGKYEQYIKWPWYVWLGFIAGLIAIVMVTILLCCSNGSLQCRICI
PDI-SARS-COV-1 H1cCT-AA
(SEQ ID NO: 97)
MAKNVAIFGLLFSLLVLVPSQIFASDLDRCTTFDDVQAPNYTQHTSSMRGVYYPDEIFRSDT
LYLTQDLFLPFYSNVTGFHTINHTFGNPVIPFKDGIYFAATEKSNVVRGWVFGSTMNNKSQS
VIIINNSTNVVIRACNFELCDNPFFAVSKPMGTQTHTMIFDNAFNCTFEYISDAFSLDVSEK
SGNFKHLREFVFKNKDGFLYVYKGYQPIDVVRDLPSGFNTLKPIFKLPLGINITNFRAILTA
FSPAQDIWGTSAAAYFVGYLKPTTFMLKYDENGTITDAVDCSQNPLAELKCSVKSFEIDKGI
YQTSNFRVVPSGDVVRFPNITNLCPFGEVFNATKFPSVYAWERKKISNCVADYSVLYNSTFF
STFKCYGVSATKLNDLCFSNVYADSFVVKGDDVRQIAPGQTGVIADYNYKLPDDFMGCVLAW
NTRNIDATSTGNYNYKYRYLRHGKLRPFERDISNVPFSPDGKPCTPPALNCYWPLNDYGFYT
TTGIGYQPYRVVVLSFELLNAPATVCGPKLSTDLIKNQCVNFNFNGLTGTGVLTPSSKRFQP
FQQFGRDVSDFTDSVRDPKTSEILDISPCSFGGVSVITPGTNASSEVAVLYQDVNCTDVSTA
IHADQLTPAWRIYSTGNNVFQTQAGCLIGAEHVDTSYECDIPIGAGICASYHTVSLLASTSQ
KSIVAYTMSLGADSSIAYSNNTIAIPTNFSISITTEVMPVSMAKTSVDCNMYICGDSTECAN
LLLQYGSFCTQLNRALSGIAAEQDRNTREVFAQVKQMYKTPTLKYFGGFNFSQILPDPLKPT
KRSFIEDLLFNKVTLADAGFMKQYGECLGDINARDLICAQKFNGLTVLPPLLTDDMIAAYTA
ALVSGTATAGWTFGAGAALQIPFAMQMAYRFNGIGVTQNVLYENQKQIANQFNKAISQIQES
LTTTSTALGKLQDVVNQNAQALNTLVKQLSSNFGAISSVLNDILSRLDPPEAEVQIDRLITG
RLQSLQTYVTQQLIRAAEIRASANLAATKMSECVLGQSKRVDFCGKGYHLMSFPQAAPHGVV
FLHVTYVPSQERNFTTAPAICHEGKAYFPREGVFVFNGTSWFITQRNFFSPQIITTDNTFVS
GNCDVVIGIINNTVYDPLQPELDSFKEELDKYFKNHTSPDVDLGDISGINASVVNIQKEIDR
LNEVAKNLNESLIDLQELGKYEQYIKWPWYVWLGFIAGLIAIVMVTILLSFWMCSNGSLQCR
ICI
IF(AvB + wtCT-MERS).r
(SEQ ID NO: 98)
ACGACACGACTAAGGCCTTCAGTGAACGTGGACCTTGTGAGGCTCAAGGTCATACTCCTC
IF(H1cCT-wtTM).r
(SEQ ID NO: 99)
ACGACACGACTAAGGCCTTCAAATACATATTCTACACTGTAGAGACCCA
IF(H5ITMCT).r
(SEQ ID NO: 100)
ACGACACGACTAAGGCCTTCAAATGCAAATTCTGCATTGTAACGATCC
PDI-MERS-wtTMCT-DNA
(SEQ ID NO: 101)
atggcgaaaaacgttgcgattttcggcttattgttttctcttcttgtgttggttccttctca
gatcttcgcgTATGTCGATGTGGGTCCCGATAGTGTTAAGTCCGCCTGCATCGAAGTGGACA
TTCAGCAGACCTTCTTCGATAAGACTTGGCCTCGGCCAATTGATGTGTCCAAGGCCGACGGC
ATTATCTACCCCCAAGGTCGGACATATTCCAACATAACTATCACCTATCAGGGGCTATTCCC
TTATCAGGGCGACCATGGGGACATGTACGTTTACAGCGCTGGTCACGCTACAGGGACGACCC
CCCAGAAGCTCTTCGTGGCGAACTATAGTCAGGACGTGAAACAGTTTGCCAACGGTTTTGTA
GTGCGCATCGGGGCAGCCGCTAACTCCACTGGTACTGTTATTATCAGCCCTTCCACGAGTGC
CACAATTCGAAAGATCTATCCGGCCTTCATGCTAGGATCCTCTGTGGGCAATTTTAGCGACG
GTAAGATGGGTCGGTTCTTCAACCACACGCTTGTGCTGCTTCCCGATGGGTGCGGTACTTTG
CTGAGGGCCTTTTACTGTATCCTAGAGCCCCGATCCGGCAACCACTGCCCCGCCGGGAACTC
GTATACTTCCTTTGCCACTTATCATACTCCAGCCACGGATTGTAGCGATGGGAACTACAATA
GGAACGCCAGTTTGAATTCCTTTAAAGAGTACTTCAACTTGCGGAATTGTACCTTCATGTAT
ACATATAACATTACTGAGGACGAAATTCTCGAATGGTTCGGAATCACTCAAACAGCCCAGGG
AGTGCACCTCTTTAGTTCTCGCTATGTGGACTTATATGGAGGCAATATGTTTCAATTCGCCA
CCTTACCCGTCTACGATACGATCAAGTATTACTCGATCATACCCCACTCCATTAGGTCCATT
CAGAGCGATCGCAAGGCATGGGCCGCATTCTATGTGTATAAGCTCCAGCCCCTGACCTTCCT
CTTGGATTTCTCCGTGGACGGCTACATCAGAAGGGCTATCGATTGCGGGTTCAACGACCTCA
GCCAGCTGCATTGTTCTTATGAGAGCTTTGACGTGGAAAGCGGAGTTTACTCAGTCTCTTCC
TTTGAGGCTAAACCTTCAGGTAGCGTCGTAGAGCAAGCAGAGGGTGTGGAGTGCGATTTCTC
ACCACTGCTCAGCGGAACCCCACCCCAGGTCTACAACTTTAAGCGGCTCGTGTTCACAAACT
GTAACTATAACTTGACTAAGTTGCTGTCACTCTTTTCCGTGAATGATTTTACATGCTCCCAA
ATCAGCCCAGCCGCTATTGCGTCTAATTGCTATTCCTCATTGATCCTGGATTACTTCAGTTA
CCCCCTCTCTATGAAGAGCGATCTCTCGGTTAGTAGCGCTGGGCCTATTTCCCAGTTTAACT
ACAAACAATCCTTTTCCAATCCAACATGCCTGATCTTAGCTACTGTACCCCACAACCTGACT
ACTATTACGAAGCCACTCAAGTACTCATACATTAATAAGTGCAGCCGATTCCTCAGTGATGA
TCGCACCGAAGTGCCGCAGCTTGTAAACGCGAACCAGTACTCCCCATGCGTCTCTATTGTGC
CTTCTACAGTGTGGGAAGACGGCGATTATTATAGAAAGCAGCTGTCGCCACTGGAAGGTGGC
GGGTGGCTAGTTGCCAGTGGGTCCACAGTTGCCATGACCGAGCAACTTCAGATGGGGTTTGG
CATAACAGTGCAGTATGGTACCGATACGAACAGCGTGTGTCCAAAATTGGAATTTGCTAACG
ACACCAAGATCGCCTCCCAGTTGGGAAATTGTGTTGAATATTCCCTGTACGGAGTGTCAGGC
CGGGGGGTGTTCCAAAATTGCACCGCCGTGGGAGTGAGGCAGCAAAGATTCGTGTACGACGC
ATACCAGAATCTAGTCGGATACTATTCTGACGATGGAAACTACTACTGTCTGCGCGCTTGCG
TCTCAGTGCCCGTGAGTGTCATATATGATAAGGAGACCAAGACTCACGCTACTCTCTTTGGT
TCTGTCGCGTGCGAACACATTTCCTCTACAATGTCCCAGTATAGTCGCTCCACTCGGTCTAT
GTTAAAGCGCAGAGACAGTACCTACGGCCCTCTACAGACACCTGTGGGGTGCGTTCTCGGCC
TTGTCAATTCTAGCCTGTTTGTGGAGGATTGTAAGCTGCCCCTTGGTCAAAGCTTATGCGCA
CTGCCCGATACGCCCAGCACACTTACACCAGCTTCAGTGGGGTCCGTCCCCGGGGAAATGAG
ATTGGCCTCGATCGCTTTCAACCACCCCATACAGGTGGATCAGCTCAACTCGTCATACTTCA
AGCTAAGCATCCCTACTAATTTCTCCTTTGGTGTGACTCAGGAGTACATTCAGACCACAATT
CAAAAGGTGACCGTTGACTGCAAGCAGTATGTGTGCAACGGGTTCCAGAAATGTGAACAGCT
GCTCCGGGAGTATGGCCAGTTCTGTTCTAAAATCAACCAGGCCCTCCACGGAGCAAACCTTA
GGCAGGACGATTCTGTCAGAAACCTCTTTGCCAGCGTCAAGAGTTCTCAGAGTTCCCCTATT
ATACCTGGCTTCGGCGGGGATTTCAACCTGACACTACTTGAACCTGTAAGCATATCAACCGG
AAGTCGCAGTGCCCGTTCCGCCATCGAGGATCTGCTCTTCGACAAAGTAACTATTGCAGATC
CCGGATACATGCAGGGGTATGACGACTGCATGCAGCAGGGTCCAGCCTCTGCAAGGGATCTG
ATATGCGCACAGTATGTCGCTGGGTACAAAGTGTTGCCTCCTCTCATGGACGTGAACATGGA
AGCGGCCTATACCTCCTCACTTCTAGGCTCCATAGCGGGCGTGGGATGGACCGCAGGGCTTT
CAAGCTTCGCCGCAATTCCCTTTGCTCAATCTATCTTCTACAGGCTTAATGGCGTTGGAATC
ACCCAGCAGGTGTTAAGCGAAAACCAGAAATTGATTGCCAATAAGTTTAACCAAGCTTTGGG
GGCCATGCAGACAGGCTTTACAACCACAAACGAGGCTTTCCATAAAGTACAGGATGCGGTAA
ACAATAACGCACAAGCCCTGTCAAAGCTGGCTTCAGAGCTCTCAAATACATTTGGCGCTATA
TCCGCGTCTATCGGCGATATCATACAACGGTTGGACCCACCCGAACAGGACGCACAGATTGA
TCGTTTGATCAACGGGAGGCTTACCACCTTAAACGCTTTTGTGGCCCAGCAACTGGTGCGGT
CTGAGAGCGCCGCCTTGAGCGCTCAGCTGGCAAAGGATAAAGTGAATGAATGCGTGAAAGCT
CAATCAAAGAGAAGTGGGTTTTGTGGGCAGGGTACTCATATTGTTTCCTTTGTGGTGAACGC
CCCAAATGGACTCTACTTTATGCATGTTGGATACTACCCGAGCAACCACATCGAGGTCGTTT
CCGCCTATGGGCTTTGTGACGCAGCAAACCCTACTAACTGTATCGCGCCAGTTAATGGCTAC
TTTATTAAAACAAATAACACACGCATTGTGGATGAATGGAGTTACACAGGGTCCAGCTTCTA
CGCTCCAGAGCCTATCACCTCTCTGAACACAAAGTATGTGGCACCTCAGGTCACATATCAGA
ACATCTCGACAAACCTGCCCCCCCCACTCTTGGGCAACTCCACAGGGATCGACTTCCAGGAC
GAGCTTGACGAATTCTTCAAGAACGTGTCCACCAGTATCCCTAATTTTGGTTCGCTGACCCA
AATTAACACAACCCTGCTCGATCTGACATATGAAATGCTTTCACTACAGCAGGTGGTCAAAG
CGTTGAACGAGTCGTATATCGACCTGAAAGAGTTAGGGAATTACACATACTATAACAAATGG
CCCTGGTATATTTGGTTAGGATTCATTGCCGGGCTGGTGGCCCTTGCCTTGTGCGTATTTTT
CATCTTGTGCTGTACCGGTTGCGGTACGAATTGCATGGGAAAACTGAAATGTAATCGGTGCT
GCGATCGCTATGAGGAGTATGACCTTGAGCCTCACAAGGTCCACGTTCACTGA
PDI-MERS-H5iTMCT-DNA
(SEQ ID NO: 102)
atggcgaaaaacgttgcgattttcggcttattgttttctcttcttgtgttggttccttctca
gatcttcgcgTATGTCGATGTGGGTCCCGATAGTGTTAAGTCCGCCTGCATCGAAGTGGACA
TTCAGCAGACCTTCTTCGATAAGACTTGGCCTCGGCCAATTGATGTGTCCAAGGCCGACGGC
ATTATCTACCCCCAAGGTCGGACATATTCCAACATAACTATCACCTATCAGGGGCTATTCCC
TTATCAGGGCGACCATGGGGACATGTACGTTTACAGCGCTGGTCACGCTACAGGGACGACCC
CCCAGAAGCTCTTCGTGGCGAACTATAGTCAGGACGTGAAACAGTTTGCCAACGGTTTTGTA
GTGCGCATCGGGGCAGCCGCTAACTCCACTGGTACTGTTATTATCAGCCCTTCCACGAGTGC
CACAATTCGAAAGATCTATCCGGCCTTCATGCTAGGATCCTCTGTGGGCAATTTTAGCGACG
GTAAGATGGGTCGGTTCTTCAACCACACGCTTGTGCTGCTTCCCGATGGGTGCGGTACTTTG
CTGAGGGCCTTTTACTGTATCCTAGAGCCCCGATCCGGCAACCACTGCCCCGCCGGGAACTC
GTATACTTCCTTTGCCACTTATCATACTCCAGCCACGGATTGTAGCGATGGGAACTACAATA
GGAACGCCAGTTTGAATTCCTTTAAAGAGTACTTCAACTTGCGGAATTGTACCTTCATGTAT
ACATATAACATTACTGAGGACGAAATTCTCGAATGGTTCGGAATCACTCAAACAGCCCAGGG
AGTGCACCTCTTTAGTTCTCGCTATGTGGACTTATATGGAGGCAATATGTTTCAATTCGCCA
CCTTACCCGTCTACGATACGATCAAGTATTACTCGATCATACCCCACTCCATTAGGTCCATT
CAGAGCGATCGCAAGGCATGGGCCGCATTCTATGTGTATAAGCTCCAGCCCCTGACCTTCCT
CTTGGATTTCTCCGTGGACGGCTACATCAGAAGGGCTATCGATTGCGGGTTCAACGACCTCA
GCCAGCTGCATTGTTCTTATGAGAGCTTTGACGTGGAAAGCGGAGTTTACTCAGTCTCTTCC
TTTGAGGCTAAACCTTCAGGTAGCGTCGTAGAGCAAGCAGAGGGTGTGGAGTGCGATTTCTC
ACCACTGCTCAGCGGAACCCCACCCCAGGTCTACAACTTTAAGCGGCTCGTGTTCACAAACT
GTAACTATAACTTGACTAAGTTGCTGTCACTCTTTTCCGTGAATGATTTTACATGCTCCCAA
ATCAGCCCAGCCGCTATTGCGTCTAATTGCTATTCCTCATTGATCCTGGATTACTTCAGTTA
CCCCCTCTCTATGAAGAGCGATCTCTCGGTTAGTAGCGCTGGGCCTATTTCCCAGTTTAACT
ACAAACAATCCTTTTCCAATCCAACATGCCTGATCTTAGCTACTGTACCCCACAACCTGACT
ACTATTACGAAGCCACTCAAGTACTCATACATTAATAAGTGCAGCCGATTCCTCAGTGATGA
TCGCACCGAAGTGCCGCAGCTTGTAAACGCGAACCAGTACTCCCCATGCGTCTCTATTGTGC
CTTCTACAGTGTGGGAAGACGGCGATTATTATAGAAAGCAGCTGTCGCCACTGGAAGGTGGC
GGGTGGCTAGTTGCCAGTGGGTCCACAGTTGCCATGACCGAGCAACTTCAGATGGGGTTTGG
CATAACAGTGCAGTATGGTACCGATACGAACAGCGTGTGTCCAAAATTGGAATTTGCTAACG
ACACCAAGATCGCCTCCCAGTTGGGAAATTGTGTTGAATATTCCCTGTACGGAGTGTCAGGC
CGGGGGGTGTTCCAAAATTGCACCGCCGTGGGAGTGAGGCAGCAAAGATTCGTGTACGACGC
ATACCAGAATCTAGTCGGATACTATTCTGACGATGGAAACTACTACTGTCTGCGCGCTTGCG
TCTCAGTGCCCGTGAGTGTCATATATGATAAGGAGACCAAGACTCACGCTACTCTCTTTGGT
TCTGTCGCGTGCGAACACATTTCCTCTACAATGTCCCAGTATAGTCGCTCCACTCGGTCTAT
GTTAAAGCGCAGAGACAGTACCTACGGCCCTCTACAGACACCTGTGGGGTGCGTTCTCGGCC
TTGTCAATTCTAGCCTGTTTGTGGAGGATTGTAAGCTGCCCCTTGGTCAAAGCTTATGCGCA
CTGCCCGATACGCCCAGCACACTTACACCAGCTTCAGTGGGGTCCGTCCCCGGGGAAATGAG
ATTGGCCTCGATCGCTTTCAACCACCCCATACAGGTGGATCAGCTCAACTCGTCATACTTCA
AGCTAAGCATCCCTACTAATTTCTCCTTTGGTGTGACTCAGGAGTACATTCAGACCACAATT
CAAAAGGTGACCGTTGACTGCAAGCAGTATGTGTGCAACGGGTTCCAGAAATGTGAACAGCT
GCTCCGGGAGTATGGCCAGTTCTGTTCTAAAATCAACCAGGCCCTCCACGGAGCAAACCTTA
GGCAGGACGATTCTGTCAGAAACCTCTTTGCCAGCGTCAAGAGTTCTCAGAGTTCCCCTATT
ATACCTGGCTTCGGCGGGGATTTCAACCTGACACTACTTGAACCTGTAAGCATATCAACCGG
AAGTCGCAGTGCCCGTTCCGCCATCGAGGATCTGCTCTTCGACAAAGTAACTATTGCAGATC
CCGGATACATGCAGGGGTATGACGACTGCATGCAGCAGGGTCCAGCCTCTGCAAGGGATCTG
ATATGCGCACAGTATGTCGCTGGGTACAAAGTGTTGCCTCCTCTCATGGACGTGAACATGGA
AGCGGCCTATACCTCCTCACTTCTAGGCTCCATAGCGGGCGTGGGATGGACCGCAGGGCTTT
CAAGCTTCGCCGCAATTCCCTTTGCTCAATCTATCTTCTACAGGCTTAATGGCGTTGGAATC
ACCCAGCAGGTGTTAAGCGAAAACCAGAAATTGATTGCCAATAAGTTTAACCAAGCTTTGGG
GGCCATGCAGACAGGCTTTACAACCACAAACGAGGCTTTCCATAAAGTACAGGATGCGGTAA
ACAATAACGCACAAGCCCTGTCAAAGCTGGCTTCAGAGCTCTCAAATACATTTGGCGCTATA
TCCGCGTCTATCGGCGATATCATACAACGGTTGGACCCACCCGAACAGGACGCACAGATTGA
TCGTTTGATCAACGGGAGGCTTACCACCTTAAACGCTTTTGTGGCCCAGCAACTGGTGCGGT
CTGAGAGCGCCGCCTTGAGCGCTCAGCTGGCAAAGGATAAAGTGAATGAATGCGTGAAAGCT
CAATCAAAGAGAAGTGGGTTTTGTGGGCAGGGTACTCATATTGTTTCCTTTGTGGTGAACGC
CCCAAATGGACTCTACTTTATGCATGTTGGATACTACCCGAGCAACCACATCGAGGTCGTTT
CCGCCTATGGGCTTTGTGACGCAGCAAACCCTACTAACTGTATCGCGCCAGTTAATGGCTAC
TTTATTAAAACAAATAACACACGCATTGTGGATGAATGGAGTTACACAGGGTCCAGCTTCTA
CGCTCCAGAGCCTATCACCTCTCTGAACACAAAGTATGTGGCACCTCAGGTCACATATCAGA
ACATCTCGACAAACCTGCCCCCCCCACTCTTGGGCAACTCCACAGGGATCGACTTCCAGGAC
GAGCTTGACGAATTCTTCAAGAACGTGTCCACCAGTATCCCTAATTTTGGTTCGCTGACCCA
AATTAACACAACCCTGCTCGATCTGACATATGAAATGCTTTCACTACAGCAGGTGGTCAAAG
CGTTGAACGAGTCGTATATCGACCTGAAAGAGTTAGGGAATTACACATACTATAACAAATGG
CCCTGGTATcaaatactgtcaatttattcaacagtggcgagttccctagcactggcaatcat
gatggctggtctatctttatggatgtgctccaatggatcgttacaatgcagaatttgcattT
GA
PDI-MERS-H5iCT-DNA
(SEQ ID NO: 103)
atggcgaaaaacgttgcgattttcggcttattgttttctcttcttgtgttggttccttctca
gatcttcgcgTATGTCGATGTGGGTCCCGATAGTGTTAAGTCCGCCTGCATCGAAGTGGACA
TTCAGCAGACCTTCTTCGATAAGACTTGGCCTCGGCCAATTGATGTGTCCAAGGCCGACGGC
ATTATCTACCCCCAAGGTCGGACATATTCCAACATAACTATCACCTATCAGGGGCTATTCCC
TTATCAGGGCGACCATGGGGACATGTACGTTTACAGCGCTGGTCACGCTACAGGGACGACCC
CCCAGAAGCTCTTCGTGGCGAACTATAGTCAGGACGTGAAACAGTTTGCCAACGGTTTTGTA
GTGCGCATCGGGGCAGCCGCTAACTCCACTGGTACTGTTATTATCAGCCCTTCCACGAGTGC
CACAATTCGAAAGATCTATCCGGCCTTCATGCTAGGATCCTCTGTGGGCAATTTTAGCGACG
GTAAGATGGGTCGGTTCTTCAACCACACGCTTGTGCTGCTTCCCGATGGGTGCGGTACTTTG
CTGAGGGCCTTTTACTGTATCCTAGAGCCCCGATCCGGCAACCACTGCCCCGCCGGGAACTC
GTATACTTCCTTTGCCACTTATCATACTCCAGCCACGGATTGTAGCGATGGGAACTACAATA
GGAACGCCAGTTTGAATTCCTTTAAAGAGTACTTCAACTTGCGGAATTGTACCTTCATGTAT
ACATATAACATTACTGAGGACGAAATTCTCGAATGGTTCGGAATCACTCAAACAGCCCAGGG
AGTGCACCTCTTTAGTTCTCGCTATGTGGACTTATATGGAGGCAATATGTTTCAATTCGCCA
CCTTACCCGTCTACGATACGATCAAGTATTACTCGATCATACCCCACTCCATTAGGTCCATT
CAGAGCGATCGCAAGGCATGGGCCGCATTCTATGTGTATAAGCTCCAGCCCCTGACCTTCCT
CTTGGATTTCTCCGTGGACGGCTACATCAGAAGGGCTATCGATTGCGGGTTCAACGACCTCA
GCCAGCTGCATTGTTCTTATGAGAGCTTTGACGTGGAAAGCGGAGTTTACTCAGTCTCTTCC
TTTGAGGCTAAACCTTCAGGTAGCGTCGTAGAGCAAGCAGAGGGTGTGGAGTGCGATTTCTC
ACCACTGCTCAGCGGAACCCCACCCCAGGTCTACAACTTTAAGCGGCTCGTGTTCACAAACT
GTAACTATAACTTGACTAAGTTGCTGTCACTCTTTTCCGTGAATGATTTTACATGCTCCCAA
ATCAGCCCAGCCGCTATTGCGTCTAATTGCTATTCCTCATTGATCCTGGATTACTTCAGTTA
CCCCCTCTCTATGAAGAGCGATCTCTCGGTTAGTAGCGCTGGGCCTATTTCCCAGTTTAACT
ACAAACAATCCTTTTCCAATCCAACATGCCTGATCTTAGCTACTGTACCCCACAACCTGACT
ACTATTACGAAGCCACTCAAGTACTCATACATTAATAAGTGCAGCCGATTCCTCAGTGATGA
TCGCACCGAAGTGCCGCAGCTTGTAAACGCGAACCAGTACTCCCCATGCGTCTCTATTGTGC
CTTCTACAGTGTGGGAAGACGGCGATTATTATAGAAAGCAGCTGTCGCCACTGGAAGGTGGC
GGGTGGCTAGTTGCCAGTGGGTCCACAGTTGCCATGACCGAGCAACTTCAGATGGGGTTTGG
CATAACAGTGCAGTATGGTACCGATACGAACAGCGTGTGTCCAAAATTGGAATTTGCTAACG
ACACCAAGATCGCCTCCCAGTTGGGAAATTGTGTTGAATATTCCCTGTACGGAGTGTCAGGC
CGGGGGGTGTTCCAAAATTGCACCGCCGTGGGAGTGAGGCAGCAAAGATTCGTGTACGACGC
ATACCAGAATCTAGTCGGATACTATTCTGACGATGGAAACTACTACTGTCTGCGCGCTTGCG
TCTCAGTGCCCGTGAGTGTCATATATGATAAGGAGACCAAGACTCACGCTACTCTCTTTGGT
TCTGTCGCGTGCGAACACATTTCCTCTACAATGTCCCAGTATAGTCGCTCCACTCGGTCTAT
GTTAAAGCGCAGAGACAGTACCTACGGCCCTCTACAGACACCTGTGGGGTGCGTTCTCGGCC
TTGTCAATTCTAGCCTGTTTGTGGAGGATTGTAAGCTGCCCCTTGGTCAAAGCTTATGCGCA
CTGCCCGATACGCCCAGCACACTTACACCAGCTTCAGTGGGGTCCGTCCCCGGGGAAATGAG
ATTGGCCTCGATCGCTTTCAACCACCCCATACAGGTGGATCAGCTCAACTCGTCATACTTCA
AGCTAAGCATCCCTACTAATTTCTCCTTTGGTGTGACTCAGGAGTACATTCAGACCACAATT
CAAAAGGTGACCGTTGACTGCAAGCAGTATGTGTGCAACGGGTTCCAGAAATGTGAACAGCT
GCTCCGGGAGTATGGCCAGTTCTGTTCTAAAATCAACCAGGCCCTCCACGGAGCAAACCTTA
GGCAGGACGATTCTGTCAGAAACCTCTTTGCCAGCGTCAAGAGTTCTCAGAGTTCCCCTATT
ATACCTGGCTTCGGCGGGGATTTCAACCTGACACTACTTGAACCTGTAAGCATATCAACCGG
AAGTCGCAGTGCCCGTTCCGCCATCGAGGATCTGCTCTTCGACAAAGTAACTATTGCAGATC
CCGGATACATGCAGGGGTATGACGACTGCATGCAGCAGGGTCCAGCCTCTGCAAGGGATCTG
ATATGCGCACAGTATGTCGCTGGGTACAAAGTGTTGCCTCCTCTCATGGACGTGAACATGGA
AGCGGCCTATACCTCCTCACTTCTAGGCTCCATAGCGGGCGTGGGATGGACCGCAGGGCTTT
CAAGCTTCGCCGCAATTCCCTTTGCTCAATCTATCTTCTACAGGCTTAATGGCGTTGGAATC
ACCCAGCAGGTGTTAAGCGAAAACCAGAAATTGATTGCCAATAAGTTTAACCAAGCTTTGGG
GGCCATGCAGACAGGCTTTACAACCACAAACGAGGCTTTCCATAAAGTACAGGATGCGGTAA
ACAATAACGCACAAGCCCTGTCAAAGCTGGCTTCAGAGCTCTCAAATACATTTGGCGCTATA
TCCGCGTCTATCGGCGATATCATACAACGGTTGGACCCACCCGAACAGGACGCACAGATTGA
TCGTTTGATCAACGGGAGGCTTACCACCTTAAACGCTTTTGTGGCCCAGCAACTGGTGCGGT
CTGAGAGCGCCGCCTTGAGCGCTCAGCTGGCAAAGGATAAAGTGAATGAATGCGTGAAAGCT
CAATCAAAGAGAAGTGGGTTTTGTGGGCAGGGTACTCATATTGTTTCCTTTGTGGTGAACGC
CCCAAATGGACTCTACTTTATGCATGTTGGATACTACCCGAGCAACCACATCGAGGTCGTTT
CCGCCTATGGGCTTTGTGACGCAGCAAACCCTACTAACTGTATCGCGCCAGTTAATGGCTAC
TTTATTAAAACAAATAACACACGCATTGTGGATGAATGGAGTTACACAGGGTCCAGCTTCTA
CGCTCCAGAGCCTATCACCTCTCTGAACACAAAGTATGTGGCACCTCAGGTCACATATCAGA
ACATCTCGACAAACCTGCCCCCCCCACTCTTGGGCAACTCCACAGGGATCGACTTCCAGGAC
GAGCTTGACGAATTCTTCAAGAACGTGTCCACCAGTATCCCTAATTTTGGTTCGCTGACCCA
AATTAACACAACCCTGCTCGATCTGACATATGAAATGCTTTCACTACAGCAGGTGGTCAAAG
CGTTGAACGAGTCGTATATCGACCTGAAAGAGTTAGGGAATTACACATACTATAACAAATGG
CCCTGGTATATTTGGTTAGGATTCATTGCCGGGCTGGTGGCCCTTGCCTTGTGCGTATTTTT
CATCTTGtctttatggatgtgctccaatggatcgttacaatgcagaatttgcattTGA
PDI-MERS-H5iCT(V4)-DNA
(SEQ ID NO: 104)
atggcgaaaaacgttgcgattttcggcttattgttttctcttcttgtgttggttccttctca
gatcttcgcgTATGTCGATGTGGGTCCCGATAGTGTTAAGTCCGCCTGCATCGAAGTGGACA
TTCAGCAGACCTTCTTCGATAAGACTTGGCCTCGGCCAATTGATGTGTCCAAGGCCGACGGC
ATTATCTACCCCCAAGGTCGGACATATTCCAACATAACTATCACCTATCAGGGGCTATTCCC
TTATCAGGGCGACCATGGGGACATGTACGTTTACAGCGCTGGTCACGCTACAGGGACGACCC
CCCAGAAGCTCTTCGTGGCGAACTATAGTCAGGACGTGAAACAGTTTGCCAACGGTTTTGTA
GTGCGCATCGGGGCAGCCGCTAACTCCACTGGTACTGTTATTATCAGCCCTTCCACGAGTGC
CACAATTCGAAAGATCTATCCGGCCTTCATGCTAGGATCCTCTGTGGGCAATTTTAGCGACG
GTAAGATGGGTCGGTTCTTCAACCACACGCTTGTGCTGCTTCCCGATGGGTGCGGTACTTTG
CTGAGGGCCTTTTACTGTATCCTAGAGCCCCGATCCGGCAACCACTGCCCCGCCGGGAACTC
GTATACTTCCTTTGCCACTTATCATACTCCAGCCACGGATTGTAGCGATGGGAACTACAATA
GGAACGCCAGTTTGAATTCCTTTAAAGAGTACTTCAACTTGCGGAATTGTACCTTCATGTAT
ACATATAACATTACTGAGGACGAAATTCTCGAATGGTTCGGAATCACTCAAACAGCCCAGGG
AGTGCACCTCTTTAGTTCTCGCTATGTGGACTTATATGGAGGCAATATGTTTCAATTCGCCA
CCTTACCCGTCTACGATACGATCAAGTATTACTCGATCATACCCCACTCCATTAGGTCCATT
CAGAGCGATCGCAAGGCATGGGCCGCATTCTATGTGTATAAGCTCCAGCCCCTGACCTTCCT
CTTGGATTTCTCCGTGGACGGCTACATCAGAAGGGCTATCGATTGCGGGTTCAACGACCTCA
GCCAGCTGCATTGTTCTTATGAGAGCTTTGACGTGGAAAGCGGAGTTTACTCAGTCTCTTCC
TTTGAGGCTAAACCTTCAGGTAGCGTCGTAGAGCAAGCAGAGGGTGTGGAGTGCGATTTCTC
ACCACTGCTCAGCGGAACCCCACCCCAGGTCTACAACTTTAAGCGGCTCGTGTTCACAAACT
GTAACTATAACTTGACTAAGTTGCTGTCACTCTTTTCCGTGAATGATTTTACATGCTCCCAA
ATCAGCCCAGCCGCTATTGCGTCTAATTGCTATTCCTCATTGATCCTGGATTACTTCAGTTA
CCCCCTCTCTATGAAGAGCGATCTCTCGGTTAGTAGCGCTGGGCCTATTTCCCAGTTTAACT
ACAAACAATCCTTTTCCAATCCAACATGCCTGATCTTAGCTACTGTACCCCACAACCTGACT
ACTATTACGAAGCCACTCAAGTACTCATACATTAATAAGTGCAGCCGATTCCTCAGTGATGA
TCGCACCGAAGTGCCGCAGCTTGTAAACGCGAACCAGTACTCCCCATGCGTCTCTATTGTGC
CTTCTACAGTGTGGGAAGACGGCGATTATTATAGAAAGCAGCTGTCGCCACTGGAAGGTGGC
GGGTGGCTAGTTGCCAGTGGGTCCACAGTTGCCATGACCGAGCAACTTCAGATGGGGTTTGG
CATAACAGTGCAGTATGGTACCGATACGAACAGCGTGTGTCCAAAATTGGAATTTGCTAACG
ACACCAAGATCGCCTCCCAGTTGGGAAATTGTGTTGAATATTCCCTGTACGGAGTGTCAGGC
CGGGGGGTGTTCCAAAATTGCACCGCCGTGGGAGTGAGGCAGCAAAGATTCGTGTACGACGC
ATACCAGAATCTAGTCGGATACTATTCTGACGATGGAAACTACTACTGTCTGCGCGCTTGCG
TCTCAGTGCCCGTGAGTGTCATATATGATAAGGAGACCAAGACTCACGCTACTCTCTTTGGT
TCTGTCGCGTGCGAACACATTTCCTCTACAATGTCCCAGTATAGTCGCTCCACTCGGTCTAT
GTTAAAGCGCAGAGACAGTACCTACGGCCCTCTACAGACACCTGTGGGGTGCGTTCTCGGCC
TTGTCAATTCTAGCCTGTTTGTGGAGGATTGTAAGCTGCCCCTTGGTCAAAGCTTATGCGCA
CTGCCCGATACGCCCAGCACACTTACACCAGCTTCAGTGGGGTCCGTCCCCGGGGAAATGAG
ATTGGCCTCGATCGCTTTCAACCACCCCATACAGGTGGATCAGCTCAACTCGTCATACTTCA
AGCTAAGCATCCCTACTAATTTCTCCTTTGGTGTGACTCAGGAGTACATTCAGACCACAATT
CAAAAGGTGACCGTTGACTGCAAGCAGTATGTGTGCAACGGGTTCCAGAAATGTGAACAGCT
GCTCCGGGAGTATGGCCAGTTCTGTTCTAAAATCAACCAGGCCCTCCACGGAGCAAACCTTA
GGCAGGACGATTCTGTCAGAAACCTCTTTGCCAGCGTCAAGAGTTCTCAGAGTTCCCCTATT
ATACCTGGCTTCGGCGGGGATTTCAACCTGACACTACTTGAACCTGTAAGCATATCAACCGG
AAGTCGCAGTGCCCGTTCCGCCATCGAGGATCTGCTCTTCGACAAAGTAACTATTGCAGATC
CCGGATACATGCAGGGGTATGACGACTGCATGCAGCAGGGTCCAGCCTCTGCAAGGGATCTG
ATATGCGCACAGTATGTCGCTGGGTACAAAGTGTTGCCTCCTCTCATGGACGTGAACATGGA
AGCGGCCTATACCTCCTCACTTCTAGGCTCCATAGCGGGCGTGGGATGGACCGCAGGGCTTT
CAAGCTTCGCCGCAATTCCCTTTGCTCAATCTATCTTCTACAGGCTTAATGGCGTTGGAATC
ACCCAGCAGGTGTTAAGCGAAAACCAGAAATTGATTGCCAATAAGTTTAACCAAGCTTTGGG
GGCCATGCAGACAGGCTTTACAACCACAAACGAGGCTTTCCATAAAGTACAGGATGCGGTAA
ACAATAACGCACAAGCCCTGTCAAAGCTGGCTTCAGAGCTCTCAAATACATTTGGCGCTATA
TCCGCGTCTATCGGCGATATCATACAACGGTTGGACCCACCCGAACAGGACGCACAGATTGA
TCGTTTGATCAACGGGAGGCTTACCACCTTAAACGCTTTTGTGGCCCAGCAACTGGTGCGGT
CTGAGAGCGCCGCCTTGAGCGCTCAGCTGGCAAAGGATAAAGTGAATGAATGCGTGAAAGCT
CAATCAAAGAGAAGTGGGTTTTGTGGGCAGGGTACTCATATTGTTTCCTTTGTGGTGAACGC
CCCAAATGGACTCTACTTTATGCATGTTGGATACTACCCGAGCAACCACATCGAGGTCGTTT
CCGCCTATGGGCTTTGTGACGCAGCAAACCCTACTAACTGTATCGCGCCAGTTAATGGCTAC
TTTATTAAAACAAATAACACACGCATTGTGGATGAATGGAGTTACACAGGGTCCAGCTTCTA
CGCTCCAGAGCCTATCACCTCTCTGAACACAAAGTATGTGGCACCTCAGGTCACATATCAGA
ACATCTCGACAAACCTGCCCCCCCCACTCTTGGGCAACTCCACAGGGATCGACTTCCAGGAC
GAGCTTGACGAATTCTTCAAGAACGTGTCCACCAGTATCCCTAATTTTGGTTCGCTGACCCA
AATTAACACAACCCTGCTCGATCTGACATATGAAATGCTTTCACTACAGCAGGTGGTCAAAG
CGTTGAACGAGTCGTATATCGACCTGAAAGAGTTAGGGAATTACACATACTATAACAAATGG
CCCTGGTATATTTGGTTAGGATTCATTGCCGGGCTGGTGGCCCTTGCCTTGTGCGTATTTTT
CATCTTGtgctgctccaatggatcgttacaatgcagaatttgcattTGA
PDI-MERS-H1cCT-DNA
(SEQ ID NO: 105)
atggcgaaaaacgttgcgattttcggcttattgttttctcttcttgtgttggttccttctca
gatcttcgcgTATGTCGATGTGGGTCCCGATAGTGTTAAGTCCGCCTGCATCGAAGTGGACA
TTCAGCAGACCTTCTTCGATAAGACTTGGCCTCGGCCAATTGATGTGTCCAAGGCCGACGGC
ATTATCTACCCCCAAGGTCGGACATATTCCAACATAACTATCACCTATCAGGGGCTATTCCC
TTATCAGGGCGACCATGGGGACATGTACGTTTACAGCGCTGGTCACGCTACAGGGACGACCC
CCCAGAAGCTCTTCGTGGCGAACTATAGTCAGGACGTGAAACAGTTTGCCAACGGTTTTGTA
GTGCGCATCGGGGCAGCCGCTAACTCCACTGGTACTGTTATTATCAGCCCTTCCACGAGTGC
CACAATTCGAAAGATCTATCCGGCCTTCATGCTAGGATCCTCTGTGGGCAATTTTAGCGACG
GTAAGATGGGTCGGTTCTTCAACCACACGCTTGTGCTGCTTCCCGATGGGTGCGGTACTTTG
CTGAGGGCCTTTTACTGTATCCTAGAGCCCCGATCCGGCAACCACTGCCCCGCCGGGAACTC
GTATACTTCCTTTGCCACTTATCATACTCCAGCCACGGATTGTAGCGATGGGAACTACAATA
GGAACGCCAGTTTGAATTCCTTTAAAGAGTACTTCAACTTGCGGAATTGTACCTTCATGTAT
ACATATAACATTACTGAGGACGAAATTCTCGAATGGTTCGGAATCACTCAAACAGCCCAGGG
AGTGCACCTCTTTAGTTCTCGCTATGTGGACTTATATGGAGGCAATATGTTTCAATTCGCCA
CCTTACCCGTCTACGATACGATCAAGTATTACTCGATCATACCCCACTCCATTAGGTCCATT
CAGAGCGATCGCAAGGCATGGGCCGCATTCTATGTGTATAAGCTCCAGCCCCTGACCTTCCT
CTTGGATTTCTCCGTGGACGGCTACATCAGAAGGGCTATCGATTGCGGGTTCAACGACCTCA
GCCAGCTGCATTGTTCTTATGAGAGCTTTGACGTGGAAAGCGGAGTTTACTCAGTCTCTTCC
TTTGAGGCTAAACCTTCAGGTAGCGTCGTAGAGCAAGCAGAGGGTGTGGAGTGCGATTTCTC
ACCACTGCTCAGCGGAACCCCACCCCAGGTCTACAACTTTAAGCGGCTCGTGTTCACAAACT
GTAACTATAACTTGACTAAGTTGCTGTCACTCTTTTCCGTGAATGATTTTACATGCTCCCAA
ATCAGCCCAGCCGCTATTGCGTCTAATTGCTATTCCTCATTGATCCTGGATTACTTCAGTTA
CCCCCTCTCTATGAAGAGCGATCTCTCGGTTAGTAGCGCTGGGCCTATTTCCCAGTTTAACT
ACAAACAATCCTTTTCCAATCCAACATGCCTGATCTTAGCTACTGTACCCCACAACCTGACT
ACTATTACGAAGCCACTCAAGTACTCATACATTAATAAGTGCAGCCGATTCCTCAGTGATGA
TCGCACCGAAGTGCCGCAGCTTGTAAACGCGAACCAGTACTCCCCATGCGTCTCTATTGTGC
CTTCTACAGTGTGGGAAGACGGCGATTATTATAGAAAGCAGCTGTCGCCACTGGAAGGTGGC
GGGTGGCTAGTTGCCAGTGGGTCCACAGTTGCCATGACCGAGCAACTTCAGATGGGGTTTGG
CATAACAGTGCAGTATGGTACCGATACGAACAGCGTGTGTCCAAAATTGGAATTTGCTAACG
ACACCAAGATCGCCTCCCAGTTGGGAAATTGTGTTGAATATTCCCTGTACGGAGTGTCAGGC
CGGGGGGTGTTCCAAAATTGCACCGCCGTGGGAGTGAGGCAGCAAAGATTCGTGTACGACGC
ATACCAGAATCTAGTCGGATACTATTCTGACGATGGAAACTACTACTGTCTGCGCGCTTGCG
TCTCAGTGCCCGTGAGTGTCATATATGATAAGGAGACCAAGACTCACGCTACTCTCTTTGGT
TCTGTCGCGTGCGAACACATTTCCTCTACAATGTCCCAGTATAGTCGCTCCACTCGGTCTAT
GTTAAAGCGCAGAGACAGTACCTACGGCCCTCTACAGACACCTGTGGGGTGCGTTCTCGGCC
TTGTCAATTCTAGCCTGTTTGTGGAGGATTGTAAGCTGCCCCTTGGTCAAAGCTTATGCGCA
CTGCCCGATACGCCCAGCACACTTACACCAGCTTCAGTGGGGTCCGTCCCCGGGGAAATGAG
ATTGGCCTCGATCGCTTTCAACCACCCCATACAGGTGGATCAGCTCAACTCGTCATACTTCA
AGCTAAGCATCCCTACTAATTTCTCCTTTGGTGTGACTCAGGAGTACATTCAGACCACAATT
CAAAAGGTGACCGTTGACTGCAAGCAGTATGTGTGCAACGGGTTCCAGAAATGTGAACAGCT
GCTCCGGGAGTATGGCCAGTTCTGTTCTAAAATCAACCAGGCCCTCCACGGAGCAAACCTTA
GGCAGGACGATTCTGTCAGAAACCTCTTTGCCAGCGTCAAGAGTTCTCAGAGTTCCCCTATT
ATACCTGGCTTCGGCGGGGATTTCAACCTGACACTACTTGAACCTGTAAGCATATCAACCGG
AAGTCGCAGTGCCCGTTCCGCCATCGAGGATCTGCTCTTCGACAAAGTAACTATTGCAGATC
CCGGATACATGCAGGGGTATGACGACTGCATGCAGCAGGGTCCAGCCTCTGCAAGGGATCTG
ATATGCGCACAGTATGTCGCTGGGTACAAAGTGTTGCCTCCTCTCATGGACGTGAACATGGA
AGCGGCCTATACCTCCTCACTTCTAGGCTCCATAGCGGGCGTGGGATGGACCGCAGGGCTTT
CAAGCTTCGCCGCAATTCCCTTTGCTCAATCTATCTTCTACAGGCTTAATGGCGTTGGAATC
ACCCAGCAGGTGTTAAGCGAAAACCAGAAATTGATTGCCAATAAGTTTAACCAAGCTTTGGG
GGCCATGCAGACAGGCTTTACAACCACAAACGAGGCTTTCCATAAAGTACAGGATGCGGTAA
ACAATAACGCACAAGCCCTGTCAAAGCTGGCTTCAGAGCTCTCAAATACATTTGGCGCTATA
TCCGCGTCTATCGGCGATATCATACAACGGTTGGACCCACCCGAACAGGACGCACAGATTGA
TCGTTTGATCAACGGGAGGCTTACCACCTTAAACGCTTTTGTGGCCCAGCAACTGGTGCGGT
CTGAGAGCGCCGCCTTGAGCGCTCAGCTGGCAAAGGATAAAGTGAATGAATGCGTGAAAGCT
CAATCAAAGAGAAGTGGGTTTTGTGGGCAGGGTACTCATATTGTTTCCTTTGTGGTGAACGC
CCCAAATGGACTCTACTTTATGCATGTTGGATACTACCCGAGCAACCACATCGAGGTCGTTT
CCGCCTATGGGCTTTGTGACGCAGCAAACCCTACTAACTGTATCGCGCCAGTTAATGGCTAC
TTTATTAAAACAAATAACACACGCATTGTGGATGAATGGAGTTACACAGGGTCCAGCTTCTA
CGCTCCAGAGCCTATCACCTCTCTGAACACAAAGTATGTGGCACCTCAGGTCACATATCAGA
ACATCTCGACAAACCTGCCCCCCCCACTCTTGGGCAACTCCACAGGGATCGACTTCCAGGAC
GAGCTTGACGAATTCTTCAAGAACGTGTCCACCAGTATCCCTAATTTTGGTTCGCTGACCCA
AATTAACACAACCCTGCTCGATCTGACATATGAAATGCTTTCACTACAGCAGGTGGTCAAAG
CGTTGAACGAGTCGTATATCGACCTGAAAGAGTTAGGGAATTACACATACTATAACAAATGG
CCCTGGTATATTTGGTTAGGATTCATTGCCGGGCTGGTGGCCCTTGCCTTGTGCGTATTTTT
CATCTTGagcttctggatgtgctctaatgggtctctacagtgtagaatatgtattTGA
PDI-MERS-wtTMCT-AA
(SEQ ID NO: 106)
MAKNVAIFGLLFSLLVLVPSQIFAYVDVGPDSVKSACIEVDIQQTFFDKTWPRPIDVSKADG
IIYPQGRTYSNITITYQGLFPYQGDHGDMYVYSAGHATGTTPQKLFVANYSQDVKQFANGFV
VRIGAAANSTGTVIISPSTSATIRKIYPAFMLGSSVGNFSDGKMGRFFNHTLVLLPDGCGTL
LRAFYCILEPRSGNHCPAGNSYTSFATYHTPATDCSDGNYNRNASLNSFKEYFNLRNCTFMY
TYNITEDEILEWFGITQTAQGVHLFSSRYVDLYGGNMFQFATLPVYDTIKYYSIIPHSIRSI
QSDRKAWAAFYVYKLQPLTFLLDFSVDGYIRRAIDCGFNDLSQLHCSYESFDVESGVYSVSS
FEAKPSGSVVEQAEGVECDFSPLLSGTPPQVYNFKRLVFTNCNYNLTKLLSLFSVNDFTCSQ
ISPAAIASNCYSSLILDYFSYPLSMKSDLSVSSAGPISQFNYKQSFSNPTCLILATVPHNLT
TITKPLKYSYINKCSRFLSDDRTEVPQLVNANQYSPCVSIVPSTVWEDGDYYRKQLSPLEGG
GWLVASGSTVAMTEQLQMGFGITVQYGTDTNSVCPKLEFANDTKIASQLGNCVEYSLYGVSG
RGVFQNCTAVGVRQQRFVYDAYQNLVGYYSDDGNYYCLRACVSVPVSVIYDKETKTHATLFG
SVACEHISSTMSQYSRSTRSMLKRRDSTYGPLQTPVGCVLGLVNSSLFVEDCKLPLGQSLCA
LPDTPSTLTPASVGSVPGEMRLASIAFNHPIQVDQLNSSYFKLSIPTNFSFGVTQEYIQTTI
QKVTVDCKQYVCNGFQKCEQLLREYGQFCSKINQALHGANLRQDDSVRNLFASVKSSQSSPI
IPGFGGDFNLTLLEPVSISTGSRSARSAIEDLLFDKVTIADPGYMQGYDDCMQQGPASARDL
ICAQYVAGYKVLPPLMDVNMEAAYTSSLLGSIAGVGWTAGLSSFAAIPFAQSIFYRLNGVGI
TQQVLSENQKLIANKFNQALGAMQTGFTTTNEAFHKVQDAVNNNAQALSKLASELSNTFGAI
SASIGDIIQRLDPPEQDAQIDRLINGRLTTLNAFVAQQLVRSESAALSAQLAKDKVNECVKA
QSKRSGFCGQGTHIVSFVVNAPNGLYFMHVGYYPSNHIEVVSAYGLCDAANPTNCIAPVNGY
FIKTNNTRIVDEWSYTGSSFYAPEPITSLNTKYVAPQVTYQNISTNLPPPLLGNSTGIDFQD
ELDEFFKNVSTSIPNFGSLTQINTTLLDLTYEMLSLQQVVKALNESYIDLKELGNYTYYNKW
PWYIWLGFIAGLVALALCVFFILCCTGCGTNCMGKLKCNRCCDRYEEYDLEPHKVHVH
PDI-MERS-H5iTMCT-AA
(SEQ ID NO: 107)
MAKNVAIFGLLFSLLVLVPSQIFAYVDVGPDSVKSACIEVDIQQTFFDKTWPRPIDVSKADG
IIYPQGRTYSNITITYQGLFPYQGDHGDMYVYSAGHATGTTPQKLFVANYSQDVKQFANGFV
VRIGAAANSTGTVIISPSTSATIRKIYPAFMLGSSVGNFSDGKMGRFFNHTLVLLPDGCGTL
LRAFYCILEPRSGNHCPAGNSYTSFATYHTPATDCSDGNYNRNASLNSFKEYFNLRNCTFMY
TYNITEDEILEWFGITQTAQGVHLFSSRYVDLYGGNMFQFATLPVYDTIKYYSIIPHSIRSI
QSDRKAWAAFYVYKLQPLTFLLDFSVDGYIRRAIDCGFNDLSQLHCSYESFDVESGVYSVSS
FEAKPSGSVVEQAEGVECDFSPLLSGTPPQVYNFKRLVFTNCNYNLTKLLSLFSVNDFTCSQ
ISPAAIASNCYSSLILDYFSYPLSMKSDLSVSSAGPISQFNYKQSFSNPTCLILATVPHNLT
TITKPLKYSYINKCSRFLSDDRTEVPQLVNANQYSPCVSIVPSTVWEDGDYYRKQLSPLEGG
GWLVASGSTVAMTEQLQMGFGITVQYGTDTNSVCPKLEFANDTKIASQLGNCVEYSLYGVSG
RGVFQNCTAVGVRQQRFVYDAYQNLVGYYSDDGNYYCLRACVSVPVSVIYDKETKTHATLFG
SVACEHISSTMSQYSRSTRSMLKRRDSTYGPLQTPVGCVLGLVNSSLFVEDCKLPLGQSLCA
LPDTPSTLTPASVGSVPGEMRLASIAFNHPIQVDQLNSSYFKLSIPTNFSFGVTQEYIQTTI
QKVTVDCKQYVCNGFQKCEQLLREYGQFCSKINQALHGANLRODDSVRNLFASVKSSQSSPI
IPGFGGDFNLTLLEPVSISTGSRSARSAIEDLLFDKVTIADPGYMQGYDDCMQQGPASARDL
ICAQYVAGYKVLPPLMDVNMEAAYTSSLLGSIAGVGWTAGLSSFAAIPFAQSIFYRLNGVGI
TQQVLSENQKLIANKFNQALGAMQTGFTTTNEAFHKVQDAVNNNAQALSKLASELSNTFGAI
SASIGDIIQRLDPPEQDAQIDRLINGRLTTLNAFVAQQLVRSESAALSAQLAKDKVNECVKA
QSKRSGFCGQGTHIVSFVVNAPNGLYFMHVGYYPSNHIEVVSAYGLCDAANPTNCIAPVNGY
FIKTNNTRIVDEWSYTGSSFYAPEPITSLNTKYVAPQVTYQNISTNLPPPLLGNSTGIDFQD
ELDEFFKNVSTSIPNFGSLTQINTTLLDLTYEMLSLQQVVKALNESYIDLKELGNYTYYNKW
PWYQILSIYSTVASSLALAIMMAGLSLWMCSNGSLQCRICI
PDI-MERS-H5iCT-AA
(SEQ ID NO: 108)
MAKNVAIFGLLFSLLVLVPSQIFAYVDVGPDSVKSACIEVDIQQTFFDKTWPRPIDVSKADG
IIYPQGRTYSNITITYQGLFPYQGDHGDMYVYSAGHATGTTPQKLFVANYSQDVKQFANGFV
VRIGAAANSTGTVIISPSTSATIRKIYPAFMLGSSVGNFSDGKMGRFFNHTLVLLPDGCGTL
LRAFYCILEPRSGNHCPAGNSYTSFATYHTPATDCSDGNYNRNASLNSFKEYFNLRNCTFMY
TYNITEDEILEWFGITQTAQGVHLFSSRYVDLYGGNMFQFATLPVYDTIKYYSIIPHSIRSI
QSDRKAWAAFYVYKLQPLTFLLDFSVDGYIRRAIDCGFNDLSQLHCSYESFDVESGVYSVSS
FEAKPSGSVVEQAEGVECDFSPLLSGTPPQVYNFKRLVFTNCNYNLTKLLSLFSVNDFTCSQ
ISPAAIASNCYSSLILDYFSYPLSMKSDLSVSSAGPISQFNYKQSFSNPTCLILATVPHNLT
TITKPLKYSYINKCSRFLSDDRTEVPQLVNANQYSPCVSIVPSTVWEDGDYYRKQLSPLEGG
GWLVASGSTVAMTEQLQMGFGITVQYGTDTNSVCPKLEFANDTKIASQLGNCVEYSLYGVSG
RGVFQNCTAVGVRQQRFVYDAYQNLVGYYSDDGNYYCLRACVSVPVSVIYDKETKTHATLFG
SVACEHISSTMSQYSRSTRSMLKRRDSTYGPLQTPVGCVLGLVNSSLFVEDCKLPLGQSLCA
LPDTPSTLTPASVGSVPGEMRLASIAFNHPIQVDQLNSSYFKLSIPTNFSFGVTQEYIQTTI
QKVTVDCKQYVCNGFQKCEQLLREYGQFCSKINQALHGANLRQDDSVRNLFASVKSSQSSPI
IPGFGGDFNLTLLEPVSISTGSRSARSAIEDLLFDKVTIADPGYMQGYDDCMQQGPASARDL
ICAQYVAGYKVLPPLMDVNMEAAYTSSLLGSIAGVGWTAGLSSFAAIPFAQSIFYRLNGVGI
TQQVLSENQKLIANKFNQALGAMQTGFTTTNEAFHKVQDAVNNNAQALSKLASELSNTFGAI
SASIGDIIQRLDPPEQDAQIDRLINGRLTTLNAFVAQQLVRSESAALSAQLAKDKVNECVKA
QSKRSGFCGQGTHIVSFVVNAPNGLYFMHVGYYPSNHIEVVSAYGLCDAANPTNCIAPVNGY
FIKTNNTRIVDEWSYTGSSFYAPEPITSLNTKYVAPQVTYQNISTNLPPPLLGNSTGIDFQD
ELDEFFKNVSTSIPNFGSLTQINTTLLDLTYEMLSLQQVVKALNESYIDLKELGNYTYYNKW
PWYIWLGFIAGLVALALCVFFILSLWMCSNGSLQCRICI
PDI-MERS-H5iCT(V4)-AA
(SEQ ID NO: 109)
MAKNVAIFGLLFSLLVLVPSQIFAYVDVGPDSVKSACIEVDIQQTFFDKTWPRPIDVSKADG
IIYPQGRTYSNITITYQGLFPYQGDHGDMYVYSAGHATGTTPQKLFVANYSQDVKQFANGFV
VRIGAAANSTGTVIISPSTSATIRKIYPAFMLGSSVGNFSDGKMGRFFNHTLVLLPDGCGTL
LRAFYCILEPRSGNHCPAGNSYTSFATYHTPATDCSDGNYNRNASLNSFKEYFNLRNCTFMY
TYNITEDEILEWFGITQTAQGVHLFSSRYVDLYGGNMFQFATLPVYDTIKYYSIIPHSIRSI
QSDRKAWAAFYVYKLQPLTFLLDFSVDGYIRRAIDCGFNDLSQLHCSYESFDVESGVYSVSS
FEAKPSGSVVEQAEGVECDFSPLLSGTPPQVYNFKRLVFTNCNYNLTKLLSLFSVNDFTCSQ
ISPAAIASNCYSSLILDYFSYPLSMKSDLSVSSAGPISQFNYKQSFSNPTCLILATVPHNLT
TITKPLKYSYINKCSRFLSDDRTEVPQLVNANQYSPCVSIVPSTVWEDGDYYRKQLSPLEGG
GWLVASGSTVAMTEQLQMGFGITVQYGTDTNSVCPKLEFANDTKIASQLGNCVEYSLYGVSG
RGVFQNCTAVGVRQQRFVYDAYQNLVGYYSDDGNYYCLRACVSVPVSVIYDKETKTHATLFG
SVACEHISSTMSQYSRSTRSMLKRRDSTYGPLQTPVGCVLGLVNSSLFVEDCKLPLGQSLCA
LPDTPSTLTPASVGSVPGEMRLASIAFNHPIQVDQLNSSYFKLSIPTNFSFGVTQEYIQTTI
QKVTVDCKQYVCNGFQKCEQLLREYGQFCSKINQALHGANLRODDSVRNLFASVKSSQSSPI
IPGFGGDFNLTLLEPVSISTGSRSARSAIEDLLFDKVTIADPGYMQGYDDCMQQGPASARDL
ICAQYVAGYKVLPPLMDVNMEAAYTSSLLGSIAGVGWTAGLSSFAAIPFAQSIFYRLNGVGI
TQQVLSENQKLIANKFNQALGAMQTGFTTTNEAFHKVQDAVNNNAQALSKLASELSNTFGAI
SASIGDIIQRLDPPEQDAQIDRLINGRLTTLNAFVAQQLVRSESAALSAQLAKDKVNECVKA
QSKRSGFCGQGTHIVSFVVNAPNGLYFMHVGYYPSNHIEVVSAYGLCDAANPTNCIAPVNGY
FIKTNNTRIVDEWSYTGSSFYAPEPITSLNTKYVAPQVTYQNISTNLPPPLLGNSTGIDFQD
ELDEFFKNVSTSIPNFGSLTQINTTLLDLTYEMLSLQQVVKALNESYIDLKELGNYTYYNKW
PWYIWLGFIAGLVALALCVFFILCCSNGSLQCRICI
PDI-MERS-H1cCT-AA
(SEQ ID NO: 110)
MAKNVAIFGLLFSLLVLVPSQIFAYVDVGPDSVKSACIEVDIQQTFFDKTWPRPIDVSKADG
IIYPQGRTYSNITITYQGLFPYQGDHGDMYVYSAGHATGTTPQKLFVANYSQDVKQFANGFV
VRIGAAANSTGTVIISPSTSATIRKIYPAFMLGSSVGNFSDGKMGRFFNHTLVLLPDGCGTL
LRAFYCILEPRSGNHCPAGNSYTSFATYHTPATDCSDGNYNRNASLNSFKEYFNLRNCTFMY
TYNITEDEILEWFGITQTAQGVHLFSSRYVDLYGGNMFQFATLPVYDTIKYYSIIPHSIRSI
QSDRKAWAAFYVYKLQPLTFLLDESVDGYIRRAIDCGFNDLSQLHCSYESFDVESGVYSVSS
FEAKPSGSVVEQAEGVECDFSPLLSGTPPQVYNFKRLVFTNCNYNLTKLLSLFSVNDFTCSQ
ISPAAIASNCYSSLILDYFSYPLSMKSDLSVSSAGPISQFNYKQSFSNPTCLILATVPHNLT
TITKPLKYSYINKCSRFLSDDRTEVPQLVNANQYSPCVSIVPSTVWEDGDYYRKQLSPLEGG
GWLVASGSTVAMTEQLQMGFGITVQYGTDTNSVCPKLEFANDTKIASQLGNCVEYSLYGVSG
RGVFQNCTAVGVRQQRFVYDAYQNLVGYYSDDGNYYCLRACVSVPVSVIYDKETKTHATLFG
SVACEHISSTMSQYSRSTRSMLKRRDSTYGPLQTPVGCVLGLVNSSLFVEDCKLPLGQSLCA
LPDTPSTLTPASVGSVPGEMRLASIAFNHPIQVDQLNSSYFKLSIPTNFSFGVTQEYIQTTI
QKVTVDCKQYVCNGFQKCEQLLREYGQFCSKINQALHGANLRODDSVRNLFASVKSSQSSPI
IPGFGGDFNLTLLEPVSISTGSRSARSAIEDLLFDKVTIADPGYMQGYDDCMQQGPASARDL
ICAQYVAGYKVLPPLMDVNMEAAYTSSLLGSIAGVGWTAGLSSFAAIPFAQSIFYRLNGVGI
TQQVLSENQKLIANKFNQALGAMQTGFTTTNEAFHKVQDAVNNNAQALSKLASELSNTFGAI
SASIGDIIQRLDPPEQDAQIDRLINGRLTTLNAFVAQQLVRSESAALSAQLAKDKVNECVKA
QSKRSGFCGQGTHIVSFVVNAPNGLYFMHVGYYPSNHIEVVSAYGLCDAANPTNCIAPVNGY
FIKTNNTRIVDEWSYTGSSFYAPEPITSLNTKYVAPQVTYQNISTNLPPPLLGNSTGIDFQD
ELDEFFKNVSTSIPNFGSLTQINTTLLDLTYEMLSLQQVVKALNESYIDLKELGNYTYYNKW
PWYIWLGFIAGLVALALCVFFILSFWMCSNGSLQCRICI
Cloning vector 7147 from left to right T-DNA
(SEQ ID NO: 111)
tggcaggatatattgtggtgtaaacaaattgacgcttagacaacttaataacacattgcgga
cgtttttaatgtactgaattaacgccgaatcccgggctggtatatttatatgttgtcaaata
actcaaaaaccataaaagtttaagttagcaagtgtgtacatttttacttgaacaaaaatatt
cacctactactgttataaatcattattaaacattagagtaaagaaatatggatgataagaac
aagagtagtgatattttgacaacaattttgttgcaacatttgagaaaattttgttgttctct
cttttcattggtcaaaaacaatagagagagaaaaaggaagagggagaataaaaacataatgt
gagtatgagagagaaagttgtacaaaagttgtaccaaaatagttgtacaaatatcattgagg
aatttgacaaaagctacacaaataagggttaattgctgtaaataaataaggatgacgcatta
gagagatgtaccattagagaatttttggcaagtcattaaaaagaaagaataaattattttta
aaattaaaagttgagtcatttgattaaacatgtgattatttaatgaattgatgaaagagttg
gattaaagttgtattagtaattagaatttggtgtcaaatttaatttgacatttgatcttttc
ctatatattgccccatagagtcagttaactcatttttatatttcatagatcaaataagagaa
ataacggtatattaatccctccaaaaaaaaaaaacggtatatttactaaaaaatctaagcca
cgtaggaggataacaggatccccgtaggaggataacatccaatccaaccaatcacaacaatc
ctgatgagataacccactttaagcccacgcatctgtggcacatctacattatctaaatcaca
cattcttccacacatctgagccacacaaaaaccaatccacatctttatcacccattctataa
aaaatcacactttgtgagtctacactttgattcccttcaaacacatacaaagagaagagact
aattaattaattaatcatcttgagagaaaatggaacgagctatacaaggaaacgacgctagg
gaacaagctaacagtgaacgttgggatggaggatcaggaggtaccacttctcccttcaaact
tcctgacgaaagtccgagttggactgagtggcggctacataacgatgagacgaattcgaatc
aagataatccccttggtttcaaggaaagctggggtttcgggaaagttgtatttaagagatat
ctcagatacgacaggacggaagcttcactgcacagagtccttggatcttggacgggagattc
ggttaactatgcagcatctcgatttttcggtttcgaccagatcggatgtacctatagtattc
ggtttcgaggagttagtatcaccgtttctggagggtcgcgaactcttcagcatctctgtgag
atggcaattcggtctaagcaagaactgctacagcttgccccaatcgaagtggaaagtaatgt
atcaagaggatgccctgaaggtactcaaaccttcgaaaaagaaagcgagtaagttaaaatgc
ttcttcgtctcctatttataatatggtttgttattgttaattttgttcttgtagaagagctt
aattaatcgttgttgttatgaaatactatttgtatgagatgaactggtgtaatgtaattcat
ttacataagtggagtcagaatcagaatgtttcctccataactaactagacatgaagacctgc
cgcgtacaattgtcttatatttgaacaactaaaattgaacatcttttgccacaactttataa
gtggttaatatagctcaaatatatggtcaagttcaatagattaataatggaaatatcagtta
tcgaaattcattaacaatcaacttaacgttattaactactaattttatatcatcccctttga
taaatgatagtacaccaattaggaaggagcatgctcgcctaggagattgtcgtttcccgcct
tcagtttgcaagctgctctagccgtgtagccaatacgcaaaccgcctctccccgcgcgttgg
gaattactagcgcgtgtcgacaagcttgcatgccggtcaacatggtggagcacgacacactt
gtctactccaaaaatatcaaagatacagtctcagaagaccaaagggcaattgagacttttca
acaaagggtaatatccggaaacctcctcggattccattgcccagctatctgtcactttattg
tgaagatagtggaaaaggaaggtggctcctacaaatgccatcattgcgataaaggaaaggcc
atcgttgaagatgcctctgccgacagtggtcccaaagatggacccccacccacgaggagcat
cgtggaaaaagaagacgttccaaccacgtcttcaaagcaagtggattgatgtgataacatgg
tggagcacgacacacttgtctactccaaaaatatcaaagatacagtctcagaagaccaaagg
gcaattgagacttttcaacaaagggtaatatccggaaacctcctcggattccattgcccagc
tatctgtcactttattgtgaagatagtggaaaaggaaggtggctcctacaaatgccatcatt
gcgataaaggaaaggccatcgttgaagatgcctctgccgacagtggtcccaaagatggaccc
ccacccacgaggagcatcgtggaaaaagaagacgttccaaccacgtcttcaaagcaagtgga
ttgatgtgatatctccactgacgtaagggatgacgcacaatcccactatccttcgcaagacc
cttcctctatataaggaagttcatttcatttggagaggcactccatttgaatctatcaaacc
aaaacacattgagacgtcacgtactcctcagccaaaacgacacccccatctgtctatccact
ggcccctggatctgctgcccaaactaactccatggtgaccctgggatgcctggtcaagggct
atttccctgagccagtgacagtgacctggaactctggatccctgtccagcggtgtgcacacc
ttcccagctgtcctgcagtctgacctctacactctgagcagctcagtgactgtcccctccag
cacctggcccagcgagaccgtcacctgcaacgttgcccacccggccagcagcaccaaggtgg
acaagaaaattgtgcccagggattgtggttgtaagccttgcatatgtacagtcccagaagta
tcatctgtcttcatcttccccccaaagcccaaggatgtgctcaccattactctgactcctaa
ggtcacgtgtgttgtggtagacatcagcaaggatgatcccgaggtccagttcagctggtttg
tagatgatgtggaggtgcacacagctcagacgcaaccccgggaggagcagttcaacagcact
ttccgctcagtcagtgaacttcccatcatgcaccaggactggctcaatggcaaggagacgtc
cagattttggcgatctattcaactgtcgccagttcattggtactggtagtctccctgggggc
aatcagtttctggatgtgctctaatgggtctctacagtgtagaatatgtatttaaaggcctt
agtcgtgtcgtttttcaaataatataatccttttagggttttagttagtttaaattttctgt
tgctcctgtttagcaggtcgtgccttcagcaagcacacaaaaacagagtgtttattttaagt
tgtttgtttagtgattcaaaaaaaaaatcgttcaaacatttggcaataaagtttcttaagat
tgaatcctgttgccggtcttgcgatgattatcatataatttctgttgaattacgttaagcat
gtaataattaacatgtaatgcatgacgttatttatgagatgggtttttatgattagagtccc
gcaattatacatttaatacgcgatagaaaacaaaatatagcgcgcaaactaggataaattat
cgcgcgcggtgtcatctatgttactagatctctagagtctcaagcttggcgcgcccacgtga
ctagtggcactggccgtcgttttacaacgtcgtgactgggaaaaccctggcgttacccaact
taatcgccttgcagcacatccccctttcgccagctggcgtaatagcgaagaggcccgcaccg
atcgcccttcccaacagttgcgcagcctgaatggcgaatgctagagcagcttgagcttggat
cagattgtcgtttcccgccttcagtttaaactatcagtgtttgacaggatatattggcgggt
aaacctaagagaaaagagcgttta
Native SARS-CoV-1 S protein wtTM/CT AA P59594
(SEQ ID NO: 112)
MFIFLLFLTLTSGSDLDRCTTFDDVQAPNYTQHTSSMRGVYYPDEIFRSDTLYLTQDLFLPF
YSNVTGFHTINHTFGNPVIPFKDGIYFAATEKSNVVRGWVFGSTMNNKSQSVIIINNSTNVV
IRACNFELCDNPFFAVSKPMGTQTHTMIFDNAFNCTFEYISDAFSLDVSEKSGNFKHLREFV
FKNKDGFLYVYKGYQPIDVVRDLPSGFNTLKPIFKLPLGINITNFRAILTAFSPAQDIWGTS
AAAYFVGYLKPTTFMLKYDENGTITDAVDCSQNPLAELKCSVKSFEIDKGIYQTSNFRVVPS
GDVVRFPNITNLCPFGEVFNATKFPSVYAWERKKISNCVADYSVLYNSTFFSTFKCYGVSAT
KLNDLCFSNVYADSFVVKGDDVRQIAPGQTGVIADYNYKLPDDFMGCVLAWNTRNIDATSTG
NYNYKYRYLRHGKLRPFERDISNVPFSPDGKPCTPPALNCYWPLNDYGFYTTTGIGYQPYRV
VVLSFELLNAPATVCGPKLSTDLIKNQCVNFNFNGLTGTGVLTPSSKRFQPFQQFGRDVSDF
TDSVRDPKTSEILDISPCSFGGVSVITPGTNASSEVAVLYQDVNCTDVSTAIHADQLTPAWR
IYSTGNNVFQTQAGCLIGAEHVDTSYECDIPIGAGICASYHTVSLLRSTSQKSIVAYTMSLG
ADSSIAYSNNTIAIPTNFSISITTEVMPVSMAKTSVDCNMYICGDSTECANLLLQYGSFCTQ
LNRALSGIAAEQDRNTREVFAQVKQMYKTPTLKYFGGFNFSQILPDPLKPTKRSFIEDLLFN
KVTLADAGFMKQYGECLGDINARDLICAQKFNGLTVLPPLLTDDMIAAYTAALVSGTATAGW
TFGAGAALQIPFAMQMAYRFNGIGVTQNVLYENQKQIANQFNKAISQIQESLTTTSTALGKL
QDVVNQNAQALNTLVKQLSSNFGAISSVLNDILSRLDKVEAEVQIDRLITGRLQSLQTYVTQ
QLIRAAEIRASANLAATKMSECVLGQSKRVDFCGKGYHLMSFPQAAPHGVVFLHVTYVPSQE
RNFTTAPAICHEGKAYFPREGVFVFNGTSWFITQRNFFSPQIITTDNTFVSGNCDVVIGIIN
NTVYDPLQPELDSFKEELDKYFKNHTSPDVDLGDISGINASVVNIQKEIDRLNEVAKNLNES
LIDLQELGKYEQYIKWPWYVWLGFIAGLIAIVMVTILLCCMTSCCSCLKGACSCGSCCKFDE
DDSEPVLKGVKLHYT
Native MERS S protein wtTM/CT AA AFY13307
(SEQ ID NO: 113)
MIHSVFLLMFLLTPTESYVDVGPDSVKSACIEVDIQQTFFDKTWPRPIDVSKADGIIYPQGR
TYSNITITYQGLFPYQGDHGDMYVYSAGHATGTTPQKLFVANYSQDVKQFANGFVVRIGAAA
NSTGTVIISPSTSATIRKIYPAFMLGSSVGNFSDGKMGRFFNHTLVLLPDGCGTLLRAFYCI
LEPRSGNHCPAGNSYTSFATYHTPATDCSDGNYNRNASLNSFKEYFNLRNCTFMYTYNITED
EILEWFGITQTAQGVHLFSSRYVDLYGGNMFQFATLPVYDTIKYYSIIPHSIRSIQSDRKAW
AAFYVYKLQPLTFLLDFSVDGYIRRAIDCGFNDLSQLHCSYESFDVESGVYSVSSFEAKPSG
SVVEQAEGVECDFSPLLSGTPPQVYNFKRLVFTNCNYNLTKLLSLFSVNDFTCSQISPAAIA
SNCYSSLILDYFSYPLSMKSDLSVSSAGPISQFNYKQSFSNPTCLILATVPHNLTTITKPLK
YSYINKCSRFLSDDRTEVPQLVNANQYSPCVSIVPSTVWEDGDYYRKQLSPLEGGGWLVASG
STVAMTEQLQMGFGITVQYGTDTNSVCPKLEFANDTKIASQLGNCVEYSLYGVSGRGVFQNC
TAVGVRQQRFVYDAYQNLVGYYSDDGNYYCLRACVSVPVSVIYDKETKTHATLFGSVACEHI
SSTMSQYSRSTRSMLKRRDSTYGPLQTPVGCVLGLVNSSLFVEDCKLPLGQSLCALPDTPST
LTPRSVRSVPGEMRLASIAFNHPIQVDQLNSSYFKLSIPTNFSFGVTQEYIQTTIQKVTVDC
KQYVCNGFQKCEQLLREYGQFCSKINQALHGANLRQDDSVRNLFASVKSSQSSPIIPGFGGD
FNLTLLEPVSISTGSRSARSAIEDLLFDKVTIADPGYMQGYDDCMQQGPASARDLICAQYVA
GYKVLPPLMDVNMEAAYTSSLLGSIAGVGWTAGLSSFAAIPFAQSIFYRLNGVGITQQVLSE
NQKLIANKFNQALGAMQTGFTTTNEAFHKVQDAVNNNAQALSKLASELSNTFGAISASIGDI
IQRLDVLEQDAQIDRLINGRLTTLNAFVAQQLVRSESAALSAQLAKDKVNECVKAQSKRSGF
CGQGTHIVSFVVNAPNGLYFMHVGYYPSNHIEVVSAYGLCDAANPTNCIAPVNGYFIKTNNT
RIVDEWSYTGSSFYAPEPITSLNTKYVAPQVTYQNISTNLPPPLLGNSTGIDFQDELDEFFK
NVSTSIPNFGSLTQINTTLLDLTYEMLSLQQVVKALNESYIDLKELGNYTYYNKWPWYIWLG
FIAGLVALALCVFFILCCTGCGTNCMGKLKCNRCCDRYEEYDLEPHKVHVH
Native SARS-CoV-1 S protein wtTM/CT AA P59594 without signal peptide
(SEQ ID NO: 114)
SDLDRCTTFDDVQAPNYTQHTSSMRGVYYPDEIFRSDTLYLTQDLFLPFYSNVTGFHTINHT
FGNPVIPFKDGIYFAATEKSNVVRGWVFGSTMNNKSQSVIIINNSTNVVIRACNFELCDNPF
FAVSKPMGTQTHTMIFDNAFNCTFEYISDAFSLDVSEKSGNFKHLREFVFKNKDGFLYVYKG
YQPIDVVRDLPSGFNTLKPIFKLPLGINITNFRAILTAFSPAQDIWGTSAAAYFVGYLKPTT
FMLKYDENGTITDAVDCSQNPLAELKCSVKSFEIDKGIYQTSNFRVVPSGDVVRFPNITNLC
PFGEVFNATKFPSVYAWERKKISNCVADYSVLYNSTFFSTFKCYGVSATKLNDLCFSNVYAD
SFVVKGDDVRQIAPGQTGVIADYNYKLPDDFMGCVLAWNTRNIDATSTGNYNYKYRYLRHGK
LRPFERDISNVPFSPDGKPCTPPALNCYWPLNDYGFYTTTGIGYQPYRVVVLSFELLNAPAT
VCGPKLSTDLIKNQCVNFNFNGLTGTGVLTPSSKRFQPFQQFGRDVSDFTDSVRDPKTSEIL
DISPCSFGGVSVITPGTNASSEVAVLYQDVNCTDVSTAIHADQLTPAWRIYSTGNNVFQTQA
GCLIGAEHVDTSYECDIPIGAGICASYHTVSLLRSTSQKSIVAYTMSLGADSSIAYSNNTIA
IPTNFSISITTEVMPVSMAKTSVDCNMYICGDSTECANLLLQYGSFCTQLNRALSGIAAEQD
RNTREVFAQVKQMYKTPTLKYFGGFNFSQILPDPLKPTKRSFIEDLLFNKVTLADAGFMKQY
GECLGDINARDLICAQKFNGLTVLPPLLTDDMIAAYTAALVSGTATAGWTFGAGAALQIPFA
MQMAYRFNGIGVTQNVLYENQKQIANQFNKAISQIQESLTTTSTALGKLQDVVNQNAQALNT
LVKQLSSNFGAISSVLNDILSRLDKVEAEVQIDRLITGRLQSLQTYVTQQLIRAAEIRASAN
LAATKMSECVLGQSKRVDFCGKGYHLMSFPQAAPHGVVFLHVTYVPSQERNFTTAPAICHEG
KAYFPREGVFVFNGTSWFITQRNFFSPQIITTDNTFVSGNCDVVIGIINNTVYDPLQPELDS
FKEELDKYFKNHTSPDVDLGDISGINASVVNIQKEIDRLNEVAKNLNESLIDLQELGKYEQY
IKWPWYVWLGFIAGLIAIVMVTILLCCMTSCCSCLKGACSCGSCCKFDEDDSEPVLKGVKLH
YT
Native MERS S protein wtTM/CT AA AFY13307 without signal peptide
(SEQ ID NO: 115)
YVDVGPDSVKSACIEVDIQQTFFDKTWPRPIDVSKADGIIYPQGRTYSNITITYQGLFPYQG
DHGDMYVYSAGHATGTTPQKLFVANYSQDVKQFANGFVVRIGAAANSTGTVIISPSTSATIR
KIYPAFMLGSSVGNFSDGKMGRFFNHTLVLLPDGCGTLLRAFYCILEPRSGNHCPAGNSYTS
FATYHTPATDCSDGNYNRNASLNSFKEYFNLRNCTFMYTYNITEDEILEWFGITQTAQGVHL
FSSRYVDLYGGNMFQFATLPVYDTIKYYSIIPHSIRSIQSDRKAWAAFYVYKLQPLTFLLDF
SVDGYIRRAIDCGFNDLSQLHCSYESFDVESGVYSVSSFEAKPSGSVVEQAEGVECDFSPLL
SGTPPQVYNFKRLVFTNCNYNLTKLLSLFSVNDFTCSQISPAAIASNCYSSLILDYFSYPLS
MKSDLSVSSAGPISQFNYKQSFSNPTCLILATVPHNLTTITKPLKYSYINKCSRFLSDDRTE
VPQLVNANQYSPCVSIVPSTVWEDGDYYRKQLSPLEGGGWLVASGSTVAMTEQLQMGFGITV
QYGTDTNSVCPKLEFANDTKIASQLGNCVEYSLYGVSGRGVFQNCTAVGVRQORFVYDAYQN
LVGYYSDDGNYYCLRACVSVPVSVIYDKETKTHATLFGSVACEHISSTMSQYSRSTRSMLKR
RDSTYGPLQTPVGCVLGLVNSSLFVEDCKLPLGQSLCALPDTPSTLTPRSVRSVPGEMRLAS
IAFNHPIQVDQLNSSYFKLSIPTNFSFGVTQEYIQTTIQKVTVDCKQYVCNGFQKCEQLLRE
YGQFCSKINQALHGANLRQDDSVRNLFASVKSSQSSPIIPGFGGDFNLTLLEPVSISTGSRS
ARSAIEDLLFDKVTIADPGYMQGYDDCMQQGPASARDLICAQYVAGYKVLPPLMDVNMEAAY
TSSLLGSIAGVGWTAGLSSFAAIPFAQSIFYRLNGVGITQQVLSENQKLIANKFNQALGAMQ
TGFTTTNEAFHKVQDAVNNNAQALSKLASELSNTFGAISASIGDIIQRLDVLEQDAQIDRLI
NGRLTTLNAFVAQQLVRSESAALSAQLAKDKVNECVKAQSKRSGFCGQGTHIVSFVVNAPNG
LYFMHVGYYPSNHIEVVSAYGLCDAANPTNCIAPVNGYFIKTNNTRIVDEWSYTGSSFYAPE
PITSLNTKYVAPQVTYQNISTNLPPPLLGNSTGIDFQDELDEFFKNVSTSIPNFGSLTQINT
TLLDLTYEMLSLQQVVKALNESYIDLKELGNYTYYNKWPWYIWLGFIAGLVALALCVFFILC
CTGCGTNCMGKLKCNRCCDRYEEYDLEPHKVHVH
TMCT region of modified PDI-SARS-COV-1 wtTMCT-AA
(SEQ ID NO: 116)
WYVWLGFIAGLIAIVMVTILLCCMTSCCSCLKGACSCGSCCKFDEDDSEPVLKGVKLHYT
TMCT region of modified PDI-SARS-COV-1 H5iTMCT-AA
(SEQ ID NO: 117)
WYQILSIYSTVASSLALAIMMAGLSLWMCSNGSLQCRICI
TMCT region of modified PDI-SARS-COV-1 H5iCT-AA
(SEQ ID NO: 118)
WYVWLGFIAGLIAIVMVTILLSLWMCSNGSLQCRICI
TMCT region of modified PDI-SARS-COV-1 H5iCT(V4)-AA
(SEQ ID NO: 119)
WYVWLGFIAGLIAIVMVTILLCCSNGSLQCRICI
TMCT region of modified PDI-SARS-COV-1 HIcCT-AA
(SEQ ID NO: 120)
WYVWLGFIAGLIAIVMVTILLSFWMCSNGSLQCRICI
TMCT region of modified PDI-MERS-wtTMCT-AA
(SEQ ID NO: 121)
WYIWLGFIAGLVALALCVFFILCCTGCGTNCMGKLKCNRCCDRYEEYDLEPHKVHVH
TMCT region of modified PDI-MERS-H5iTMCT-AA
(SEQ ID NO: 122)
WYQILSIYSTVASSLALAIMMAGLSLWMCSNGSLQCRICI
TMCT region of modified PDI-MERS-H5iCT-AA
(SEQ ID NO: 123)
WYIWLGFIAGLVALALCVFFILSLWMCSNGSLQCRICI
TMCT region of modified PDI-MERS-H5iCT(V4)-AA
(SEQ ID NO: 124)
WYIWLGFIAGLVALALCVFFILCCSNGSLQCRICI
TMCT region of modified PDI-MERS-H1cCT-AA
(SEQ ID NO: 125)
WYIWLGFIAGLVALALCVFFILSFWMCSNGSLQCRICI
TMCT region of modified PDI-S-protein + H1 Cal
(SEQ ID NO: 126)
WYIWLGFIAGLIAIVMVTIMLSFWMCSNGSLQCRICI
TMCT region of modified PDI-S-protein + H3 Minn
(SEQ ID NO: 127)
WYIWLGFIAGLIAIVMVTIMLMWACQKGNIRCNICI
TMCT region of modified PDI-S-protein + H6 HK
(SEQ ID NO: 128)
WYIWLGFIAGLIAIVMVTIMLGLWMCSNGSMQCRICI
TMCT region of modified PDI-S-protein + H7 Guangdong
(SEQ ID NO: 129)
WYIWLGFIAGLIAIVMVTIMLVFICVKNGNMRCTICI
TMCT region of modified PDI-S-protein + H9 HK
(SEQ ID NO: 130)
WYIWLGFIAGLIAIVMVTIMLLFWAMSNGSCRCNICI
TMCT region of modified PDI-S-protein + B/ Wash
(SEQ ID NO: 131)
WYIWLGFIAGLIAIVMVTIMLVVYMVSRDNVSCSICL
Consensus Sequence of TM Domain of Coronavirus S-protein
(SEQ ID NO: 132)
WYXWLGFIAGLXAXXX{X}VXXXL ({X} may be absent)
Consensus Sequence of TM Domain of Coronavirus S-protein
(SEQ ID NO: 133)
WY[I/V]WLGFIAGL[V/I]A[L/I][A/V][L/M]{X}V[F/T][F/I)XL (wherein {X} may be C
or absent)
TM/CT Region of Modified SARS-CoV-1 S protein with intervening peptide sequence Xₙ
(SEQ ID NO: 134)
WYVWLGFIAGLIAIVMVTIL - (X)n - CSNGSXXCXICI
TM/CT Region of Modified MERS S protein with intervening peptide sequence Xₙ
(SEQ ID NO: 135)
WYIWLGFIAGLVALALCVFFIL - (X)n - CSNGSXXCXICI
IF(AvB + wtCT-OC43).r
(SEQ ID NO: 136)
ACGACACGACTAAGGCCTTCAGTCGTCATGCGAGGTCTTAATGACAAGC
PDI-OC43-wtTMCT-DNA
(SEQ ID NO: 137)
atggcgaaaaacgttgcgattttcggcttattgttttctcttcttgtgttggttccttctca
gatcttcgcgGTGATCGGCGATCTGAATTGTACCCTGGATCCCCGCCTGAAAGGGAGCTTTA
ACAACCGAGATACAGGACCCCCGTCTATATCCATAGATACAGTGGATGTTACGAACGGGCTC
GGCACCTACTATGTGCTAGACCGAGTTTATTTGAACACCACCTTATTCCTCAATGGATACTA
CCCAACTTCAGGTAGTACTTACAGAAACATGGCGCTGAAGGGTACGGATCTGCTGAGCACCC
TATGGTTTAAACCTCCCTTCCTCTCGGACTTTATTAATGGCATCTTCGCTAAGGTGAAAAAC
ACGAAGGTTTTCAAAGATGGAGTGATGTATTCAGAGTTCCCTGCGATCACCATTGGAAGTAC
CTTCGTGAATACTTCCTATAGCGTGGTGGTTCAACCACGGACAATCAACTCCACCCAGGACG
GCGTCAACAAGCTCCAGGGATTGCTGGAGGTGTCAGTCTGTCAATATAACATGTGTGAGTAC
CCACACACTATCTGTCACCCTAATCTAGGCAACCACTTTAAGGAACTGTGGCACTACGATAC
GGGGGTGGTAAGTTGCTTATATAAGAGAAATTTCACCTATGATGTTAATGCAACGTACCTGT
ACTTTCACTTCTATCAAGAAGGAGGAACTTTCTACGCATATTTCACAGATACCGGCTTTGTG
ACGAAATTCTTATTCAACGTTTACCTCGGAATGGCATTAAGCCATTATTACGTGATGCCTCT
CACTTGCATCAGACGCCCTAAGGATGGTTTTTCTCTGGAGTACTGGGTCACTCCCCTGACAC
CACGGCAGTACCTGCTTGCTTTTAACCAGGACGGTATCATTTTTAATGCCGTCGATTGTATG
AGCGATTTTATGAGCGAGATAAAGTGCAAGACCCAATCTATTGCTCCGCCCACGGGGGTGTA
CGAACTGAATGGTTACACCGTCCAGCCCGTTGCCGATGTATATAGACGGAAACCAGACCTGC
CCAATTGCAACATCGAAGCTTGGTTAAACGATAAGTCAGTGCCCTCCCCCCTCAATTGGGAG
AGGAAGACTTTCTCCAACTGTAATTTCAACATGTCAAGCCTGATGTCTTTCATTCAAGCCGA
TTCGTTCACTTGTAATAATATAGATGCAGCAAAGATCTATGGTATGTGCTTCAGTTCCATCA
CAATAGATAAGTTTGCAATACCAAACCGTCGCAAGGTGGACCTTCAGCTCGGCAACCTGGGC
TATCTGCAGTCCAGCAATTATAGAATAGACACCACCGCCACATCATGTCAGCTGTACTATAA
CCTCCCAGCAGCGAACGTCAGTGTTAGTAGGTTCAATCCTTCTACCTGGAATAAAAGGTTTG
GATTCATCGAAGATAGTGTGTTCGTACCTCAGCCAACAGGAGTGTTCACCAATCACAGCGTG
GTCTACGCCCAACATTGCTTCAAGGCACCCAAAAATTTCTGCCCATGTAGCAGTTGCTCCTG
CCCGGGTAAGAACAATGGGATCGGCACCTGCCCAGCAGGCACCAATTCACTTACATGCGATA
ATCTGTGTACACTGGATCCTATTACACTTAAGGCCCCTGATACCTACAAATGCCCCCAGAGC
AAGAGCCTGGTCGGTATCGGAGAACACTGTTCCGGACTTGCAGTAAAAAGCGACTATTGTGG
AAATAACTCTTGCACTTGTCAGCCACAAGCCTTCCTCGGTTGGTCCGCTGACTCTTGTTTAC
AAGGGGATAAGTGTAACATCTTCGCAAATTTCATCTTACACGATGTGAATAACGGCTTAACA
TGCAGCACAGATCTCCAGAAGGCAAACACAGAGATCGAATTAGGAGTCTGCGTTAATTACGA
TCTCTACGGGATCTCTGGCCAGGGCATCTTCGTGGAGGTTAATGCTACCTACTACAATAGTT
GGCAAAATCTGCTCTACGATAGCAATGGCAACCTCTATGGATTCAGAGACTATATTACTAAC
AGGACGTTCATGATTCACTCGTGCTATTCCGGGCGGGTGTCAGCAGCTTATCACGCAAATTC
TTCAGAGCCAGCTCTGCTATTCCGAAACATAAAATGTAATTACGTGTTCAATAATTCACTGA
CTCGGCAGCTGCAGCCGATTAATTACAGCTTCGACAGCTACCTTGGTTGCGTTGTTAACGCC
TACAACTCCACTGCCATATCAGTTCAGACCTGCGACCTTACTGTGGGCTCTGGCTATTGTGT
CGATTATTCAAAGAACGGGGGGAGCGGGTCCGCAATAACAACTGGCTATAGGTTCACCAATT
TTGAGCCTTTCACCGTGAATAGTGTCAACGATAGCCTGGAGCCTGTCGGAGGTCTTTATGAG
ATACAAATCCCCTCCGAGTTCACAATTGGCAACATGGAAGAGTTCATCCAGACGAGTTCCCC
AAAGGTGACGATCGATTGCGCGGCTTTCGTCTGCGGCGACTACGCCGCATGCAAGTTACAAC
TCGTTGAGTATGGAAGTTTTTGCGATAATATAAACGCAATTCTGACTGAAGTGAACGAACTG
CTGGACACCACTCAGTTGCAGGTGGCAAATTCGCTCATGAACGGCGTGACACTGTCAACCAA
ACTGAAGGACGGTGTCAATTTCAATGTGGATGACATTAACTTCAGCCCCGTACTGGGCTGTT
TGGGTAGTGAGTGTTCTAAGGCTAGCAGCCGCTCCGCCATTGAGGACTTGTTGTTTGATAAA
GTTAAGCTGAGTGACGTTGGATTTGTTGAGGCGTATAATAACTGTACCGGTGGTGCAGAGAT
AAGGGATCTGATCTGTGTCCAGAGTTATAAGGGGATTAAGGTTCTCCCCCCGCTACTCTCGG
AGAATCAGATATCAGGATACACCCTGGCCGCTACCTCAGCCTCGCTGTTTCCCCCTTGGACC
GCTGCCGCCGGTGTCCCATTTTATTTGAATGTGCAGTATCGGATCAACGGTCTGGGAGTGAC
AATGGACGTGCTGTCTCAGAACCAGAAACTGATCGCCAATGCATTCAACAATGCTCTGCACG
CCATCCAGCAAGGGTTTGACGCTACAAATTCTGCCCTCGTAAAAATCCAGGCCGTGGTGAAT
GCTAACGCCGAAGCCCTTAATAATCTGCTCCAGCAGCTTTCTAACCGCTTTGGAGCTATTTC
TGCCTCACTGCAGGAAATTCTATCCAGACTGGATCCCCCTGAGGCAGAAGCCCAAATCGACC
GTCTCATAAACGGCAGACTCACTGCTCTTAACGCCTACGTTAGTCAACAATTGAGCGATTCG
ACCTTGGTGAAATTCAGCGCAGCTCAGGCTATGGAGAAGGTGAACGAGTGCGTGAAGTCACA
GAGCTCCAGAATCAATTTCTGTGGCAATGGGAACCATATCATCTCCTTGGTTCAGAATGCTC
CCTACGGCCTGTATTTCATCCACTTCAACTACGTGCCCACGAAGTACGTTACAGCCAAAGTG
TCCCCCGGACTGTGCATCGCTGGTAACAGGGGCATTGCACCAAAATCCGGCTACTTCGTCAA
TGTCAACAACACATGGATGTATACTGGGAGTGGTTATTATTACCCTGAACCTATAACAGAGA
ACAATGTAGTAGTCATGTCCACATGCGCCGTCAATTATACTAAGGCCCCCTATGTTATGCTC
AACACTTCAATTCCCAATCTCCCGGATTTCAAAGAAGAGCTGGATCAGTGGTTTAAGAATCA
GACATCCGTGGCCCCTGACTTAAGCTTGGATTATATCAATGTGACTTTTTTAGACTTACAGG
TCGAGATGAACCGACTCCAGGAAGCTATAAAAGTACTGAACCACTCCTATATCAATCTGAAA
GATATCGGTACATACGAATATTACGTAAAATGGCCTTGGTATGTGTGGCTACTAATTTGCCT
TGCGGGCGTGGCTATGCTGGTCCTGCTGTTCTTCATTTGCTGCTGTACTGGGTGCGGTACTT
CCTGCTTCAAAAAGTGCGGTGGTTGCTGCGACGACTACACTGGGTACCAGGAGCTTGTCATT
AAGACCTCGCATGACGACTGA
PDI-OC43-H5iTMCT-DNA
(SEQ ID NO: 138)
atggcgaaaaacgttgcgattttcggcttattgttttctcttcttgtgttggttccttctca
gatcttcgcgGTGATCGGCGATCTGAATTGTACCCTGGATCCCCGCCTGAAAGGGAGCTTTA
ACAACCGAGATACAGGACCCCCGTCTATATCCATAGATACAGTGGATGTTACGAACGGGCTC
GGCACCTACTATGTGCTAGACCGAGTTTATTTGAACACCACCTTATTCCTCAATGGATACTA
CCCAACTTCAGGTAGTACTTACAGAAACATGGCGCTGAAGGGTACGGATCTGCTGAGCACCC
TATGGTTTAAACCTCCCTTCCTCTCGGACTTTATTAATGGCATCTTCGCTAAGGTGAAAAAC
ACGAAGGTTTTCAAAGATGGAGTGATGTATTCAGAGTTCCCTGCGATCACCATTGGAAGTAC
CTTCGTGAATACTTCCTATAGCGTGGTGGTTCAACCACGGACAATCAACTCCACCCAGGACG
GCGTCAACAAGCTCCAGGGATTGCTGGAGGTGTCAGTCTGTCAATATAACATGTGTGAGTAC
CCACACACTATCTGTCACCCTAATCTAGGCAACCACTTTAAGGAACTGTGGCACTACGATAC
GGGGGTGGTAAGTTGCTTATATAAGAGAAATTTCACCTATGATGTTAATGCAACGTACCTGT
ACTTTCACTTCTATCAAGAAGGAGGAACTTTCTACGCATATTTCACAGATACCGGCTTTGTG
ACGAAATTCTTATTCAACGTTTACCTCGGAATGGCATTAAGCCATTATTACGTGATGCCTCT
CACTTGCATCAGACGCCCTAAGGATGGTTTTTCTCTGGAGTACTGGGTCACTCCCCTGACAC
CACGGCAGTACCTGCTTGCTTTTAACCAGGACGGTATCATTTTTAATGCCGTCGATTGTATG
AGCGATTTTATGAGCGAGATAAAGTGCAAGACCCAATCTATTGCTCCGCCCACGGGGGTGTA
CGAACTGAATGGTTACACCGTCCAGCCCGTTGCCGATGTATATAGACGGAAACCAGACCTGC
CCAATTGCAACATCGAAGCTTGGTTAAACGATAAGTCAGTGCCCTCCCCCCTCAATTGGGAG
AGGAAGACTTTCTCCAACTGTAATTTCAACATGTCAAGCCTGATGTCTTTCATTCAAGCCGA
TTCGTTCACTTGTAATAATATAGATGCAGCAAAGATCTATGGTATGTGCTTCAGTTCCATCA
CAATAGATAAGTTTGCAATACCAAACCGTCGCAAGGTGGACCTTCAGCTCGGCAACCTGGGC
TATCTGCAGTCCAGCAATTATAGAATAGACACCACCGCCACATCATGTCAGCTGTACTATAA
CCTCCCAGCAGCGAACGTCAGTGTTAGTAGGTTCAATCCTTCTACCTGGAATAAAAGGTTTG
GATTCATCGAAGATAGTGTGTTCGTACCTCAGCCAACAGGAGTGTTCACCAATCACAGCGTG
GTCTACGCCCAACATTGCTTCAAGGCACCCAAAAATTTCTGCCCATGTAGCAGTTGCTCCTG
CCCGGGTAAGAACAATGGGATCGGCACCTGCCCAGCAGGCACCAATTCACTTACATGCGATA
ATCTGTGTACACTGGATCCTATTACACTTAAGGCCCCTGATACCTACAAATGCCCCCAGAGC
AAGAGCCTGGTCGGTATCGGAGAACACTGTTCCGGACTTGCAGTAAAAAGCGACTATTGTGG
AAATAACTCTTGCACTTGTCAGCCACAAGCCTTCCTCGGTTGGTCCGCTGACTCTTGTTTAC
AAGGGGATAAGTGTAACATCTTCGCAAATTTCATCTTACACGATGTGAATAACGGCTTAACA
TGCAGCACAGATCTCCAGAAGGCAAACACAGAGATCGAATTAGGAGTCTGCGTTAATTACGA
TCTCTACGGGATCTCTGGCCAGGGCATCTTCGTGGAGGTTAATGCTACCTACTACAATAGTT
GGCAAAATCTGCTCTACGATAGCAATGGCAACCTCTATGGATTCAGAGACTATATTACTAAC
AGGACGTTCATGATTCACTCGTGCTATTCCGGGCGGGTGTCAGCAGCTTATCACGCAAATTC
TTCAGAGCCAGCTCTGCTATTCCGAAACATAAAATGTAATTACGTGTTCAATAATTCACTGA
CTCGGCAGCTGCAGCCGATTAATTACAGCTTCGACAGCTACCTTGGTTGCGTTGTTAACGCC
TACAACTCCACTGCCATATCAGTTCAGACCTGCGACCTTACTGTGGGCTCTGGCTATTGTGT
CGATTATTCAAAGAACGGGGGGAGCGGGTCCGCAATAACAACTGGCTATAGGTTCACCAATT
TTGAGCCTTTCACCGTGAATAGTGTCAACGATAGCCTGGAGCCTGTCGGAGGTCTTTATGAG
ATACAAATCCCCTCCGAGTTCACAATTGGCAACATGGAAGAGTTCATCCAGACGAGTTCCCC
AAAGGTGACGATCGATTGCGCGGCTTTCGTCTGCGGCGACTACGCCGCATGCAAGTTACAAC
TCGTTGAGTATGGAAGTTTTTGCGATAATATAAACGCAATTCTGACTGAAGTGAACGAACTG
CTGGACACCACTCAGTTGCAGGTGGCAAATTCGCTCATGAACGGCGTGACACTGTCAACCAA
ACTGAAGGACGGTGTCAATTTCAATGTGGATGACATTAACTTCAGCCCCGTACTGGGCTGTT
TGGGTAGTGAGTGTTCTAAGGCTAGCAGCCGCTCCGCCATTGAGGACTTGTTGTTTGATAAA
GTTAAGCTGAGTGACGTTGGATTTGTTGAGGCGTATAATAACTGTACCGGTGGTGCAGAGAT
AAGGGATCTGATCTGTGTCCAGAGTTATAAGGGGATTAAGGTTCTCCCCCCGCTACTCTCGG
AGAATCAGATATCAGGATACACCCTGGCCGCTACCTCAGCCTCGCTGTTTCCCCCTTGGACC
GCTGCCGCCGGTGTCCCATTTTATTTGAATGTGCAGTATCGGATCAACGGTCTGGGAGTGAC
AATGGACGTGCTGTCTCAGAACCAGAAACTGATCGCCAATGCATTCAACAATGCTCTGCACG
CCATCCAGCAAGGGTTTGACGCTACAAATTCTGCCCTCGTAAAAATCCAGGCCGTGGTGAAT
GCTAACGCCGAAGCCCTTAATAATCTGCTCCAGCAGCTTTCTAACCGCTTTGGAGCTATTTC
TGCCTCACTGCAGGAAATTCTATCCAGACTGGATCCCCCTGAGGCAGAAGCCCAAATCGACC
GTCTCATAAACGGCAGACTCACTGCTCTTAACGCCTACGTTAGTCAACAATTGAGCGATTCG
ACCTTGGTGAAATTCAGCGCAGCTCAGGCTATGGAGAAGGTGAACGAGTGCGTGAAGTCACA
GAGCTCCAGAATCAATTTCTGTGGCAATGGGAACCATATCATCTCCTTGGTTCAGAATGCTC
CCTACGGCCTGTATTTCATCCACTTCAACTACGTGCCCACGAAGTACGTTACAGCCAAAGTG
TCCCCCGGACTGTGCATCGCTGGTAACAGGGGCATTGCACCAAAATCCGGCTACTTCGTCAA
TGTCAACAACACATGGATGTATACTGGGAGTGGTTATTATTACCCTGAACCTATAACAGAGA
ACAATGTAGTAGTCATGTCCACATGCGCCGTCAATTATACTAAGGCCCCCTATGTTATGCTC
AACACTTCAATTCCCAATCTCCCGGATTTCAAAGAAGAGCTGGATCAGTGGTTTAAGAATCA
GACATCCGTGGCCCCTGACTTAAGCTTGGATTATATCAATGTGACTTTTTTAGACTTACAGG
TCGAGATGAACCGACTCCAGGAAGCTATAAAAGTACTGAACCACTCCTATATCAATCTGAAA
GATATCGGTACATACGAATATTACGTAAAATGGCCTTGGTATcaaatactgtcaatttattc
aacagtggcgagttccctagcactggcaatcatgatggctggtctatctttatggatgtgct
ccaatggatcgttacaatgcagaatttgcattTGA
PDI-OC43-H5iCT-DNA
(SEQ ID NO: 139)
atggcgaaaaacgttgcgattttcggcttattgttttctcttcttgtgttggttccttctca
gatcttcgcgGTGATCGGCGATCTGAATTGTACCCTGGATCCCCGCCTGAAAGGGAGCTTTA
ACAACCGAGATACAGGACCCCCGTCTATATCCATAGATACAGTGGATGTTACGAACGGGCTC
GGCACCTACTATGTGCTAGACCGAGTTTATTTGAACACCACCTTATTCCTCAATGGATACTA
CCCAACTTCAGGTAGTACTTACAGAAACATGGCGCTGAAGGGTACGGATCTGCTGAGCACCC
TATGGTTTAAACCTCCCTTCCTCTCGGACTTTATTAATGGCATCTTCGCTAAGGTGAAAAAC
ACGAAGGTTTTCAAAGATGGAGTGATGTATTCAGAGTTCCCTGCGATCACCATTGGAAGTAC
CTTCGTGAATACTTCCTATAGCGTGGTGGTTCAACCACGGACAATCAACTCCACCCAGGACG
GCGTCAACAAGCTCCAGGGATTGCTGGAGGTGTCAGTCTGTCAATATAACATGTGTGAGTAC
CCACACACTATCTGTCACCCTAATCTAGGCAACCACTTTAAGGAACTGTGGCACTACGATAC
GGGGGTGGTAAGTTGCTTATATAAGAGAAATTTCACCTATGATGTTAATGCAACGTACCTGT
ACTTTCACTTCTATCAAGAAGGAGGAACTTTCTACGCATATTTCACAGATACCGGCTTTGTG
ACGAAATTCTTATTCAACGTTTACCTCGGAATGGCATTAAGCCATTATTACGTGATGCCTCT
CACTTGCATCAGACGCCCTAAGGATGGTTTTTCTCTGGAGTACTGGGTCACTCCCCTGACAC
CACGGCAGTACCTGCTTGCTTTTAACCAGGACGGTATCATTTTTAATGCCGTCGATTGTATG
AGCGATTTTATGAGCGAGATAAAGTGCAAGACCCAATCTATTGCTCCGCCCACGGGGGTGTA
CGAACTGAATGGTTACACCGTCCAGCCCGTTGCCGATGTATATAGACGGAAACCAGACCTGC
CCAATTGCAACATCGAAGCTTGGTTAAACGATAAGTCAGTGCCCTCCCCCCTCAATTGGGAG
AGGAAGACTTTCTCCAACTGTAATTTCAACATGTCAAGCCTGATGTCTTTCATTCAAGCCGA
TTCGTTCACTTGTAATAATATAGATGCAGCAAAGATCTATGGTATGTGCTTCAGTTCCATCA
CAATAGATAAGTTTGCAATACCAAACCGTCGCAAGGTGGACCTTCAGCTCGGCAACCTGGGC
TATCTGCAGTCCAGCAATTATAGAATAGACACCACCGCCACATCATGTCAGCTGTACTATAA
CCTCCCAGCAGCGAACGTCAGTGTTAGTAGGTTCAATCCTTCTACCTGGAATAAAAGGTTTG
GATTCATCGAAGATAGTGTGTTCGTACCTCAGCCAACAGGAGTGTTCACCAATCACAGCGTG
GTCTACGCCCAACATTGCTTCAAGGCACCCAAAAATTTCTGCCCATGTAGCAGTTGCTCCTG
CCCGGGTAAGAACAATGGGATCGGCACCTGCCCAGCAGGCACCAATTCACTTACATGCGATA
ATCTGTGTACACTGGATCCTATTACACTTAAGGCCCCTGATACCTACAAATGCCCCCAGAGC
AAGAGCCTGGTCGGTATCGGAGAACACTGTTCCGGACTTGCAGTAAAAAGCGACTATTGTGG
AAATAACTCTTGCACTTGTCAGCCACAAGCCTTCCTCGGTTGGTCCGCTGACTCTTGTTTAC
AAGGGGATAAGTGTAACATCTTCGCAAATTTCATCTTACACGATGTGAATAACGGCTTAACA
TGCAGCACAGATCTCCAGAAGGCAAACACAGAGATCGAATTAGGAGTCTGCGTTAATTACGA
TCTCTACGGGATCTCTGGCCAGGGCATCTTCGTGGAGGTTAATGCTACCTACTACAATAGTT
GGCAAAATCTGCTCTACGATAGCAATGGCAACCTCTATGGATTCAGAGACTATATTACTAAC
AGGACGTTCATGATTCACTCGTGCTATTCCGGGCGGGTGTCAGCAGCTTATCACGCAAATTC
TTCAGAGCCAGCTCTGCTATTCCGAAACATAAAATGTAATTACGTGTTCAATAATTCACTGA
CTCGGCAGCTGCAGCCGATTAATTACAGCTTCGACAGCTACCTTGGTTGCGTTGTTAACGCC
TACAACTCCACTGCCATATCAGTTCAGACCTGCGACCTTACTGTGGGCTCTGGCTATTGTGT
CGATTATTCAAAGAACGGGGGGAGCGGGTCCGCAATAACAACTGGCTATAGGTTCACCAATT
TTGAGCCTTTCACCGTGAATAGTGTCAACGATAGCCTGGAGCCTGTCGGAGGTCTTTATGAG
ATACAAATCCCCTCCGAGTTCACAATTGGCAACATGGAAGAGTTCATCCAGACGAGTTCCCC
AAAGGTGACGATCGATTGCGCGGCTTTCGTCTGCGGCGACTACGCCGCATGCAAGTTACAAC
TCGTTGAGTATGGAAGTTTTTGCGATAATATAAACGCAATTCTGACTGAAGTGAACGAACTG
CTGGACACCACTCAGTTGCAGGTGGCAAATTCGCTCATGAACGGCGTGACACTGTCAACCAA
ACTGAAGGACGGTGTCAATTTCAATGTGGATGACATTAACTTCAGCCCCGTACTGGGCTGTT
TGGGTAGTGAGTGTTCTAAGGCTAGCAGCCGCTCCGCCATTGAGGACTTGTTGTTTGATAAA
GTTAAGCTGAGTGACGTTGGATTTGTTGAGGCGTATAATAACTGTACCGGTGGTGCAGAGAT
AAGGGATCTGATCTGTGTCCAGAGTTATAAGGGGATTAAGGTTCTCCCCCCGCTACTCTCGG
AGAATCAGATATCAGGATACACCCTGGCCGCTACCTCAGCCTCGCTGTTTCCCCCTTGGACC
GCTGCCGCCGGTGTCCCATTTTATTTGAATGTGCAGTATCGGATCAACGGTCTGGGAGTGAC
AATGGACGTGCTGTCTCAGAACCAGAAACTGATCGCCAATGCATTCAACAATGCTCTGCACG
CCATCCAGCAAGGGTTTGACGCTACAAATTCTGCCCTCGTAAAAATCCAGGCCGTGGTGAAT
GCTAACGCCGAAGCCCTTAATAATCTGCTCCAGCAGCTTTCTAACCGCTTTGGAGCTATTTC
TGCCTCACTGCAGGAAATTCTATCCAGACTGGATCCCCCTGAGGCAGAAGCCCAAATCGACC
GTCTCATAAACGGCAGACTCACTGCTCTTAACGCCTACGTTAGTCAACAATTGAGCGATTCG
ACCTTGGTGAAATTCAGCGCAGCTCAGGCTATGGAGAAGGTGAACGAGTGCGTGAAGTCACA
GAGCTCCAGAATCAATTTCTGTGGCAATGGGAACCATATCATCTCCTTGGTTCAGAATGCTC
CCTACGGCCTGTATTTCATCCACTTCAACTACGTGCCCACGAAGTACGTTACAGCCAAAGTG
TCCCCCGGACTGTGCATCGCTGGTAACAGGGGCATTGCACCAAAATCCGGCTACTTCGTCAA
TGTCAACAACACATGGATGTATACTGGGAGTGGTTATTATTACCCTGAACCTATAACAGAGA
ACAATGTAGTAGTCATGTCCACATGCGCCGTCAATTATACTAAGGCCCCCTATGTTATGCTC
AACACTTCAATTCCCAATCTCCCGGATTTCAAAGAAGAGCTGGATCAGTGGTTTAAGAATCA
GACATCCGTGGCCCCTGACTTAAGCTTGGATTATATCAATGTGACTTTTTTAGACTTACAGG
TCGAGATGAACCGACTCCAGGAAGCTATAAAAGTACTGAACCACTCCTATATCAATCTGAAA
GATATCGGTACATACGAATATTACGTAAAATGGCCTTGGTATGTGTGGCTACTAATTTGCCT
TGCGGGCGTGGCTATGCTGGTCCTGCTGTTCTTCATTtctttatggatgtgctccaatggat
cgttacaatgcagaatttgcattTGA
PDI-OC43-H5iCT(V4)-DNA
(SEQ ID NO: 140)
atggcgaaaaacgttgcgattttcggcttattgttttctcttcttgtgttggttccttctca
gatcttcgcgGTGATCGGCGATCTGAATTGTACCCTGGATCCCCGCCTGAAAGGGAGCTTTA
ACAACCGAGATACAGGACCCCCGTCTATATCCATAGATACAGTGGATGTTACGAACGGGCTC
GGCACCTACTATGTGCTAGACCGAGTTTATTTGAACACCACCTTATTCCTCAATGGATACTA
CCCAACTTCAGGTAGTACTTACAGAAACATGGCGCTGAAGGGTACGGATCTGCTGAGCACCC
TATGGTTTAAACCTCCCTTCCTCTCGGACTTTATTAATGGCATCTTCGCTAAGGTGAAAAAC
ACGAAGGTTTTCAAAGATGGAGTGATGTATTCAGAGTTCCCTGCGATCACCATTGGAAGTAC
CTTCGTGAATACTTCCTATAGCGTGGTGGTTCAACCACGGACAATCAACTCCACCCAGGACG
GCGTCAACAAGCTCCAGGGATTGCTGGAGGTGTCAGTCTGTCAATATAACATGTGTGAGTAC
CCACACACTATCTGTCACCCTAATCTAGGCAACCACTTTAAGGAACTGTGGCACTACGATAC
GGGGGTGGTAAGTTGCTTATATAAGAGAAATTTCACCTATGATGTTAATGCAACGTACCTGT
ACTTTCACTTCTATCAAGAAGGAGGAACTTTCTACGCATATTTCACAGATACCGGCTTTGTG
ACGAAATTCTTATTCAACGTTTACCTCGGAATGGCATTAAGCCATTATTACGTGATGCCTCT
CACTTGCATCAGACGCCCTAAGGATGGTTTTTCTCTGGAGTACTGGGTCACTCCCCTGACAC
CACGGCAGTACCTGCTTGCTTTTAACCAGGACGGTATCATTTTTAATGCCGTCGATTGTATG
AGCGATTTTATGAGCGAGATAAAGTGCAAGACCCAATCTATTGCTCCGCCCACGGGGGTGTA
CGAACTGAATGGTTACACCGTCCAGCCCGTTGCCGATGTATATAGACGGAAACCAGACCTGC
CCAATTGCAACATCGAAGCTTGGTTAAACGATAAGTCAGTGCCCTCCCCCCTCAATTGGGAG
AGGAAGACTTTCTCCAACTGTAATTTCAACATGTCAAGCCTGATGTCTTTCATTCAAGCCGA
TTCGTTCACTTGTAATAATATAGATGCAGCAAAGATCTATGGTATGTGCTTCAGTTCCATCA
CAATAGATAAGTTTGCAATACCAAACCGTCGCAAGGTGGACCTTCAGCTCGGCAACCTGGGC
TATCTGCAGTCCAGCAATTATAGAATAGACACCACCGCCACATCATGTCAGCTGTACTATAA
CCTCCCAGCAGCGAACGTCAGTGTTAGTAGGTTCAATCCTTCTACCTGGAATAAAAGGTTTG
GATTCATCGAAGATAGTGTGTTCGTACCTCAGCCAACAGGAGTGTTCACCAATCACAGCGTG
GTCTACGCCCAACATTGCTTCAAGGCACCCAAAAATTTCTGCCCATGTAGCAGTTGCTCCTG
CCCGGGTAAGAACAATGGGATCGGCACCTGCCCAGCAGGCACCAATTCACTTACATGCGATA
ATCTGTGTACACTGGATCCTATTACACTTAAGGCCCCTGATACCTACAAATGCCCCCAGAGC
AAGAGCCTGGTCGGTATCGGAGAACACTGTTCCGGACTTGCAGTAAAAAGCGACTATTGTGG
AAATAACTCTTGCACTTGTCAGCCACAAGCCTTCCTCGGTTGGTCCGCTGACTCTTGTTTAC
AAGGGGATAAGTGTAACATCTTCGCAAATTTCATCTTACACGATGTGAATAACGGCTTAACA
TGCAGCACAGATCTCCAGAAGGCAAACACAGAGATCGAATTAGGAGTCTGCGTTAATTACGA
TCTCTACGGGATCTCTGGCCAGGGCATCTTCGTGGAGGTTAATGCTACCTACTACAATAGTT
GGCAAAATCTGCTCTACGATAGCAATGGCAACCTCTATGGATTCAGAGACTATATTACTAAC
AGGACGTTCATGATTCACTCGTGCTATTCCGGGCGGGTGTCAGCAGCTTATCACGCAAATTC
TTCAGAGCCAGCTCTGCTATTCCGAAACATAAAATGTAATTACGTGTTCAATAATTCACTGA
CTCGGCAGCTGCAGCCGATTAATTACAGCTTCGACAGCTACCTTGGTTGCGTTGTTAACGCC
TACAACTCCACTGCCATATCAGTTCAGACCTGCGACCTTACTGTGGGCTCTGGCTATTGTGT
CGATTATTCAAAGAACGGGGGGAGCGGGTCCGCAATAACAACTGGCTATAGGTTCACCAATT
TTGAGCCTTTCACCGTGAATAGTGTCAACGATAGCCTGGAGCCTGTCGGAGGTCTTTATGAG
ATACAAATCCCCTCCGAGTTCACAATTGGCAACATGGAAGAGTTCATCCAGACGAGTTCCCC
AAAGGTGACGATCGATTGCGCGGCTTTCGTCTGCGGCGACTACGCCGCATGCAAGTTACAAC
TCGTTGAGTATGGAAGTTTTTGCGATAATATAAACGCAATTCTGACTGAAGTGAACGAACTG
CTGGACACCACTCAGTTGCAGGTGGCAAATTCGCTCATGAACGGCGTGACACTGTCAACCAA
ACTGAAGGACGGTGTCAATTTCAATGTGGATGACATTAACTTCAGCCCCGTACTGGGCTGTT
TGGGTAGTGAGTGTTCTAAGGCTAGCAGCCGCTCCGCCATTGAGGACTTGTTGTTTGATAAA
GTTAAGCTGAGTGACGTTGGATTTGTTGAGGCGTATAATAACTGTACCGGTGGTGCAGAGAT
AAGGGATCTGATCTGTGTCCAGAGTTATAAGGGGATTAAGGTTCTCCCCCCGCTACTCTCGG
AGAATCAGATATCAGGATACACCCTGGCCGCTACCTCAGCCTCGCTGTTTCCCCCTTGGACC
GCTGCCGCCGGTGTCCCATTTTATTTGAATGTGCAGTATCGGATCAACGGTCTGGGAGTGAC
AATGGACGTGCTGTCTCAGAACCAGAAACTGATCGCCAATGCATTCAACAATGCTCTGCACG
CCATCCAGCAAGGGTTTGACGCTACAAATTCTGCCCTCGTAAAAATCCAGGCCGTGGTGAAT
GCTAACGCCGAAGCCCTTAATAATCTGCTCCAGCAGCTTTCTAACCGCTTTGGAGCTATTTC
TGCCTCACTGCAGGAAATTCTATCCAGACTGGATCCCCCTGAGGCAGAAGCCCAAATCGACC
GTCTCATAAACGGCAGACTCACTGCTCTTAACGCCTACGTTAGTCAACAATTGAGCGATTCG
ACCTTGGTGAAATTCAGCGCAGCTCAGGCTATGGAGAAGGTGAACGAGTGCGTGAAGTCACA
GAGCTCCAGAATCAATTTCTGTGGCAATGGGAACCATATCATCTCCTTGGTTCAGAATGCTC
CCTACGGCCTGTATTTCATCCACTTCAACTACGTGCCCACGAAGTACGTTACAGCCAAAGTG
TCCCCCGGACTGTGCATCGCTGGTAACAGGGGCATTGCACCAAAATCCGGCTACTTCGTCAA
TGTCAACAACACATGGATGTATACTGGGAGTGGTTATTATTACCCTGAACCTATAACAGAGA
ACAATGTAGTAGTCATGTCCACATGCGCCGTCAATTATACTAAGGCCCCCTATGTTATGCTC
AACACTTCAATTCCCAATCTCCCGGATTTCAAAGAAGAGCTGGATCAGTGGTTTAAGAATCA
GACATCCGTGGCCCCTGACTTAAGCTTGGATTATATCAATGTGACTTTTTTAGACTTACAGG
TCGAGATGAACCGACTCCAGGAAGCTATAAAAGTACTGAACCACTCCTATATCAATCTGAAA
GATATCGGTACATACGAATATTACGTAAAATGGCCTTGGTATGTGTGGCTACTAATTTGCCT
TGCGGGCGTGGCTATGCTGGTCCTGCTGTTCTTCATTtgctgctccaatggatcgttacaat
gcagaatttgcattTGA
PDI-OC43-H1cCT-DNA
(SEQ ID NO: 141)
atggcgaaaaacgttgcgattttcggcttattgttttctcttcttgtgttggttccttctca
gatcttcgcgGTGATCGGCGATCTGAATTGTACCCTGGATCCCCGCCTGAAAGGGAGCTTTA
ACAACCGAGATACAGGACCCCCGTCTATATCCATAGATACAGTGGATGTTACGAACGGGCTC
GGCACCTACTATGTGCTAGACCGAGTTTATTTGAACACCACCTTATTCCTCAATGGATACTA
CCCAACTTCAGGTAGTACTTACAGAAACATGGCGCTGAAGGGTACGGATCTGCTGAGCACCC
TATGGTTTAAACCTCCCTTCCTCTCGGACTTTATTAATGGCATCTTCGCTAAGGTGAAAAAC
ACGAAGGTTTTCAAAGATGGAGTGATGTATTCAGAGTTCCCTGCGATCACCATTGGAAGTAC
CTTCGTGAATACTTCCTATAGCGTGGTGGTTCAACCACGGACAATCAACTCCACCCAGGACG
GCGTCAACAAGCTCCAGGGATTGCTGGAGGTGTCAGTCTGTCAATATAACATGTGTGAGTAC
CCACACACTATCTGTCACCCTAATCTAGGCAACCACTTTAAGGAACTGTGGCACTACGATAC
GGGGGTGGTAAGTTGCTTATATAAGAGAAATTTCACCTATGATGTTAATGCAACGTACCTGT
ACTTTCACTTCTATCAAGAAGGAGGAACTTTCTACGCATATTTCACAGATACCGGCTTTGTG
ACGAAATTCTTATTCAACGTTTACCTCGGAATGGCATTAAGCCATTATTACGTGATGCCTCT
CACTTGCATCAGACGCCCTAAGGATGGTTTTTCTCTGGAGTACTGGGTCACTCCCCTGACAC
CACGGCAGTACCTGCTTGCTTTTAACCAGGACGGTATCATTTTTAATGCCGTCGATTGTATG
AGCGATTTTATGAGCGAGATAAAGTGCAAGACCCAATCTATTGCTCCGCCCACGGGGGTGTA
CGAACTGAATGGTTACACCGTCCAGCCCGTTGCCGATGTATATAGACGGAAACCAGACCTGC
CCAATTGCAACATCGAAGCTTGGTTAAACGATAAGTCAGTGCCCTCCCCCCTCAATTGGGAG
AGGAAGACTTTCTCCAACTGTAATTTCAACATGTCAAGCCTGATGTCTTTCATTCAAGCCGA
TTCGTTCACTTGTAATAATATAGATGCAGCAAAGATCTATGGTATGTGCTTCAGTTCCATCA
CAATAGATAAGTTTGCAATACCAAACCGTCGCAAGGTGGACCTTCAGCTCGGCAACCTGGGC
TATCTGCAGTCCAGCAATTATAGAATAGACACCACCGCCACATCATGTCAGCTGTACTATAA
CCTCCCAGCAGCGAACGTCAGTGTTAGTAGGTTCAATCCTTCTACCTGGAATAAAAGGTTTG
GATTCATCGAAGATAGTGTGTTCGTACCTCAGCCAACAGGAGTGTTCACCAATCACAGCGTG
GTCTACGCCCAACATTGCTTCAAGGCACCCAAAAATTTCTGCCCATGTAGCAGTTGCTCCTG
CCCGGGTAAGAACAATGGGATCGGCACCTGCCCAGCAGGCACCAATTCACTTACATGCGATA
ATCTGTGTACACTGGATCCTATTACACTTAAGGCCCCTGATACCTACAAATGCCCCCAGAGC
AAGAGCCTGGTCGGTATCGGAGAACACTGTTCCGGACTTGCAGTAAAAAGCGACTATTGTGG
AAATAACTCTTGCACTTGTCAGCCACAAGCCTTCCTCGGTTGGTCCGCTGACTCTTGTTTAC
AAGGGGATAAGTGTAACATCTTCGCAAATTTCATCTTACACGATGTGAATAACGGCTTAACA
TGCAGCACAGATCTCCAGAAGGCAAACACAGAGATCGAATTAGGAGTCTGCGTTAATTACGA
TCTCTACGGGATCTCTGGCCAGGGCATCTTCGTGGAGGTTAATGCTACCTACTACAATAGTT
GGCAAAATCTGCTCTACGATAGCAATGGCAACCTCTATGGATTCAGAGACTATATTACTAAC
AGGACGTTCATGATTCACTCGTGCTATTCCGGGCGGGTGTCAGCAGCTTATCACGCAAATTC
TTCAGAGCCAGCTCTGCTATTCCGAAACATAAAATGTAATTACGTGTTCAATAATTCACTGA
CTCGGCAGCTGCAGCCGATTAATTACAGCTTCGACAGCTACCTTGGTTGCGTTGTTAACGCC
TACAACTCCACTGCCATATCAGTTCAGACCTGCGACCTTACTGTGGGCTCTGGCTATTGTGT
CGATTATTCAAAGAACGGGGGGAGCGGGTCCGCAATAACAACTGGCTATAGGTTCACCAATT
TTGAGCCTTTCACCGTGAATAGTGTCAACGATAGCCTGGAGCCTGTCGGAGGTCTTTATGAG
ATACAAATCCCCTCCGAGTTCACAATTGGCAACATGGAAGAGTTCATCCAGACGAGTTCCCC
AAAGGTGACGATCGATTGCGCGGCTTTCGTCTGCGGCGACTACGCCGCATGCAAGTTACAAC
TCGTTGAGTATGGAAGTTTTTGCGATAATATAAACGCAATTCTGACTGAAGTGAACGAACTG
CTGGACACCACTCAGTTGCAGGTGGCAAATTCGCTCATGAACGGCGTGACACTGTCAACCAA
ACTGAAGGACGGTGTCAATTTCAATGTGGATGACATTAACTTCAGCCCCGTACTGGGCTGTT
TGGGTAGTGAGTGTTCTAAGGCTAGCAGCCGCTCCGCCATTGAGGACTTGTTGTTTGATAAA
GTTAAGCTGAGTGACGTTGGATTTGTTGAGGCGTATAATAACTGTACCGGTGGTGCAGAGAT
AAGGGATCTGATCTGTGTCCAGAGTTATAAGGGGATTAAGGTTCTCCCCCCGCTACTCTCGG
AGAATCAGATATCAGGATACACCCTGGCCGCTACCTCAGCCTCGCTGTTTCCCCCTTGGACC
GCTGCCGCCGGTGTCCCATTTTATTTGAATGTGCAGTATCGGATCAACGGTCTGGGAGTGAC
AATGGACGTGCTGTCTCAGAACCAGAAACTGATCGCCAATGCATTCAACAATGCTCTGCACG
CCATCCAGCAAGGGTTTGACGCTACAAATTCTGCCCTCGTAAAAATCCAGGCCGTGGTGAAT
GCTAACGCCGAAGCCCTTAATAATCTGCTCCAGCAGCTTTCTAACCGCTTTGGAGCTATTTC
TGCCTCACTGCAGGAAATTCTATCCAGACTGGATCCCCCTGAGGCAGAAGCCCAAATCGACC
GTCTCATAAACGGCAGACTCACTGCTCTTAACGCCTACGTTAGTCAACAATTGAGCGATTCG
ACCTTGGTGAAATTCAGCGCAGCTCAGGCTATGGAGAAGGTGAACGAGTGCGTGAAGTCACA
GAGCTCCAGAATCAATTTCTGTGGCAATGGGAACCATATCATCTCCTTGGTTCAGAATGCTC
CCTACGGCCTGTATTTCATCCACTTCAACTACGTGCCCACGAAGTACGTTACAGCCAAAGTG
TCCCCCGGACTGTGCATCGCTGGTAACAGGGGCATTGCACCAAAATCCGGCTACTTCGTCAA
TGTCAACAACACATGGATGTATACTGGGAGTGGTTATTATTACCCTGAACCTATAACAGAGA
ACAATGTAGTAGTCATGTCCACATGCGCCGTCAATTATACTAAGGCCCCCTATGTTATGCTC
AACACTTCAATTCCCAATCTCCCGGATTTCAAAGAAGAGCTGGATCAGTGGTTTAAGAATCA
GACATCCGTGGCCCCTGACTTAAGCTTGGATTATATCAATGTGACTTTTTTAGACTTACAGG
TCGAGATGAACCGACTCCAGGAAGCTATAAAAGTACTGAACCACTCCTATATCAATCTGAAA
GATATCGGTACATACGAATATTACGTAAAATGGCCTTGGTATGTGTGGCTACTAATTTGCCT
TGCGGGCGTGGCTATGCTGGTCCTGCTGTTCTTCATTagcttctggatgtgctctaatgggt
ctctacagtgtagaatatgtattTGA
PDI-OC43-wtTMCT-AA
(SEQ ID NO: 142)
MAKNVAIFGLLFSLLVLVPSQIFAVIGDLNCTLDPRLKGSFNNRDTGPPSISIDTVDVTNGL
GTYYVLDRVYLNTTLFLNGYYPTSGSTYRNMALKGTDLLSTLWFKPPFLSDFINGIFAKVKN
TKVFKDGVMYSEFPAITIGSTFVNTSYSVVVQPRTINSTQDGVNKLQGLLEVSVCQYNMCEY
PHTICHPNLGNHFKELWHYDTGVVSCLYKRNFTYDVNATYLYFHFYQEGGTFYAYFTDTGFV
TKFLFNVYLGMALSHYYVMPLTCIRRPKDGFSLEYWVTPLTPRQYLLAFNQDGIIFNAVDCM
SDFMSEIKCKTQSIAPPTGVYELNGYTVQPVADVYRRKPDLPNCNIEAWLNDKSVPSPLNWE
RKTFSNCNFNMSSLMSFIQADSFTCNNIDAAKIYGMCFSSITIDKFAIPNRRKVDLQLGNLG
YLQSSNYRIDTTATSCQLYYNLPAANVSVSRFNPSTWNKRFGFIEDSVFVPQPTGVFTNHSV
VYAQHCFKAPKNFCPCSSCSCPGKNNGIGTCPAGTNSLTCDNLCTLDPITLKAPDTYKCPQS
KSLVGIGEHCSGLAVKSDYCGNNSCTCQPQAFLGWSADSCLQGDKCNIFANFILHDVNNGLT
CSTDLQKANTEIELGVCVNYDLYGISGQGIFVEVNATYYNSWQNLLYDSNGNLYGFRDYITN
RTFMIHSCYSGRVSAAYHANSSEPALLFRNIKCNYVFNNSLTRQLQPINYSFDSYLGCVVNA
YNSTAISVQTCDLTVGSGYCVDYSKNGGSGSAITTGYRFTNFEPFTVNSVNDSLEPVGGLYE
IQIPSEFTIGNMEEFIQTSSPKVTIDCAAFVCGDYAACKLQLVEYGSFCDNINAILTEVNEL
LDTTQLQVANSLMNGVTLSTKLKDGVNFNVDDINFSPVLGCLGSECSKASSRSAIEDLLFDK
VKLSDVGFVEAYNNCTGGAEIRDLICVQSYKGIKVLPPLLSENQISGYTLAATSASLFPPWT
AAAGVPFYLNVQYRINGLGVTMDVLSQNQKLIANAFNNALHAIQQGFDATNSALVKIQAVVN
ANAEALNNLLQQLSNRFGAISASLQEILSRLDPPEAEAQIDRLINGRLTALNAYVSQQLSDS
TLVKFSAAQAMEKVNECVKSQSSRINFCGNGNHIISLVQNAPYGLYFIHFNYVPTKYVTAKV
SPGLCIAGNRGIAPKSGYFVNVNNTWMYTGSGYYYPEPITENNVVVMSTCAVNYTKAPYVML
NTSIPNLPDFKEELDQWFKNQTSVAPDLSLDYINVTFLDLQVEMNRLQEAIKVLNHSYINLK
DIGTYEYYVKWPWYVWLLICLAGVAMLVLLFFICCCTGCGTSCFKKCGGCCDDYTGYQELVI
KTSHDD
PDI-OC43-H5iTMCT-AA
(SEQ ID NO: 143)
MAKNVAIFGLLFSLLVLVPSQIFAVIGDLNCTLDPRLKGSFNNRDTGPPSISIDTVDVTNGL
GTYYVLDRVYLNTTLFLNGYYPTSGSTYRNMALKGTDLLSTLWFKPPFLSDFINGIFAKVKN
TKVFKDGVMYSEFPAITIGSTFVNTSYSVVVQPRTINSTQDGVNKLQGLLEVSVCQYNMCEY
PHTICHPNLGNHFKELWHYDTGVVSCLYKRNFTYDVNATYLYFHFYQEGGTFYAYFTDTGFV
TKFLFNVYLGMALSHYYVMPLTCIRRPKDGFSLEYWVTPLTPRQYLLAFNQDGIIFNAVDCM
SDFMSEIKCKTQSIAPPTGVYELNGYTVQPVADVYRRKPDLPNCNIEAWLNDKSVPSPLNWE
RKTFSNCNFNMSSLMSFIQADSFTCNNIDAAKIYGMCFSSITIDKFAIPNRRKVDLQLGNLG
YLQSSNYRIDTTATSCQLYYNLPAANVSVSRFNPSTWNKRFGFIEDSVFVPQPTGVFTNHSV
VYAQHCFKAPKNFCPCSSCSCPGKNNGIGTCPAGTNSLTCDNLCTLDPITLKAPDTYKCPQS
KSLVGIGEHCSGLAVKSDYCGNNSCTCQPQAFLGWSADSCLQGDKCNIFANFILHDVNNGLT
CSTDLQKANTEIELGVCVNYDLYGISGQGIFVEVNATYYNSWQNLLYDSNGNLYGFRDYITN
RTFMIHSCYSGRVSAAYHANSSEPALLFRNIKCNYVFNNSLTRQLQPINYSFDSYLGCVVNA
YNSTAISVQTCDLTVGSGYCVDYSKNGGSGSAITTGYRFTNFEPFTVNSVNDSLEPVGGLYE
IQIPSEFTIGNMEEFIQTSSPKVTIDCAAFVCGDYAACKLQLVEYGSFCDNINAILTEVNEL
LDTTQLQVANSLMNGVTLSTKLKDGVNFNVDDINFSPVLGCLGSECSKASSRSAIEDLLFDK
VKLSDVGFVEAYNNCTGGAEIRDLICVQSYKGIKVLPPLLSENQISGYTLAATSASLFPPWT
AAAGVPFYLNVQYRINGLGVTMDVLSQNQKLIANAFNNALHAIQQGFDATNSALVKIQAVVN
ANAEALNNLLQQLSNRFGAISASLQEILSRLDPPEAEAQIDRLINGRLTALNAYVSQQLSDS
TLVKFSAAQAMEKVNECVKSQSSRINFCGNGNHIISLVQNAPYGLYFIHFNYVPTKYVTAKV
SPGLCIAGNRGIAPKSGYFVNVNNTWMYTGSGYYYPEPITENNVVVMSTCAVNYTKAPYVML
NTSIPNLPDFKEELDQWFKNQTSVAPDLSLDYINVTFLDLQVEMNRLQEAIKVLNHSYINLK
DIGTYEYYVKWPWYQILSIYSTVASSLALAIMMAGLSLWMCSNGSLQCRICI
PDI-OC43-H5iCT-AA
(SEQ ID NO: 144)
MAKNVAIFGLLFSLLVLVPSQIFAVIGDLNCTLDPRLKGSFNNRDTGPPSISIDTVDVTNGL
GTYYVLDRVYLNTTLFLNGYYPTSGSTYRNMALKGTDLLSTLWFKPPFLSDFINGIFAKVKN
TKVFKDGVMYSEFPAITIGSTFVNTSYSVVVQPRTINSTQDGVNKLQGLLEVSVCQYNMCEY
PHTICHPNLGNHFKELWHYDTGVVSCLYKRNFTYDVNATYLYFHFYQEGGTFYAYFTDTGFV
TKFLFNVYLGMALSHYYVMPLTCIRRPKDGFSLEYWVTPLTPRQYLLAFNQDGIIFNAVDCM
SDFMSEIKCKTQSIAPPTGVYELNGYTVQPVADVYRRKPDLPNCNIEAWLNDKSVPSPLNWE
RKTFSNCNFNMSSLMSFIQADSFTCNNIDAAKIYGMCFSSITIDKFAIPNRRKVDLQLGNLG
YLQSSNYRIDTTATSCQLYYNLPAANVSVSRFNPSTWNKRFGFIEDSVFVPQPTGVFTNHSV
VYAQHCFKAPKNFCPCSSCSCPGKNNGIGTCPAGTNSLTCDNLCTLDPITLKAPDTYKCPQS
KSLVGIGEHCSGLAVKSDYCGNNSCTCQPQAFLGWSADSCLQGDKCNIFANFILHDVNNGLT
CSTDLQKANTEIELGVCVNYDLYGISGQGIFVEVNATYYNSWQNLLYDSNGNLYGFRDYITN
RTFMIHSCYSGRVSAAYHANSSEPALLFRNIKCNYVFNNSLTRQLQPINYSFDSYLGCVVNA
YNSTAISVQTCDLTVGSGYCVDYSKNGGSGSAITTGYRFTNFEPFTVNSVNDSLEPVGGLYE
IQIPSEFTIGNMEEFIQTSSPKVTIDCAAFVCGDYAACKLQLVEYGSFCDNINAILTEVNEL
LDTTQLQVANSLMNGVTLSTKLKDGVNFNVDDINFSPVLGCLGSECSKASSRSAIEDLLFDK
VKLSDVGFVEAYNNCTGGAEIRDLICVQSYKGIKVLPPLLSENQISGYTLAATSASLFPPWT
AAAGVPFYLNVQYRLNGLGVTMDVLSQNQKLIANAFNNALHAIQQGFDATNSALVKIQAVVN
ANAEALNNLLQQLSNRFGAISASLQEILSRLDPPEAEAQIDRLINGRLTALNAYVSQQLSDS
TLVKFSAAQAMEKVNECVKSQSSRINFCGNGNHIISLVQNAPYGLYFIHFNYVPTKYVTAKV
SPGLCIAGNRGIAPKSGYFVNVNNTWMYTGSGYYYPEPITENNVVVMSTCAVNYTKAPYVML
NTSIPNLPDFKEELDQWFKNQTSVAPDLSLDYINVTFLDLQVEMNRLQEAIKVLNHSYINLK
DIGTYEYYVKWPWYVWLLICLAGVAMLVLLFFISLWMCSNGSLQCRICI
PDI-OC43-H5iCT(V4)-AA
(SEQ ID NO: 145)
MAKNVAIFGLLFSLLVLVPSQIFAVIGDLNCTLDPRLKGSFNNRDTGPPSISIDTVDVTNGL
GTYYVLDRVYLNTTLFLNGYYPTSGSTYRNMALKGTDLLSTLWFKPPFLSDFINGIFAKVKN
TKVFKDGVMYSEFPAITIGSTFVNTSYSVVVQPRTINSTQDGVNKLQGLLEVSVCQYNMCEY
PHTICHPNLGNHFKELWHYDTGVVSCLYKRNFTYDVNATYLYFHFYQEGGTFYAYFTDTGFV
TKFLFNVYLGMALSHYYVMPLTCIRRPKDGFSLEYWVTPLTPRQYLLAFNQDGIIFNAVDCM
SDFMSEIKCKTQSIAPPTGVYELNGYTVQPVADVYRRKPDLPNCNIEAWLNDKSVPSPLNWE
RKTFSNCNFNMSSLMSFIQADSFTCNNIDAAKIYGMCFSSITIDKFAIPNRRKVDLQLGNLG
YLQSSNYRIDTTATSCQLYYNLPAANVSVSRFNPSTWNKRFGFIEDSVFVPQPTGVFTNHSV
VYAQHCFKAPKNFCPCSSCSCPGKNNGIGTCPAGTNSLTCDNLCTLDPITLKAPDTYKCPQS
KSLVGIGEHCSGLAVKSDYCGNNSCTCQPQAFLGWSADSCLQGDKCNIFANFILHDVNNGLT
CSTDLQKANTEIELGVCVNYDLYGISGQGIFVEVNATYYNSWQNLLYDSNGNLYGFRDYITN
RTFMIHSCYSGRVSAAYHANSSEPALLFRNIKCNYVFNNSLTRQLQPINYSFDSYLGCVVNA
YNSTAISVQTCDLTVGSGYCVDYSKNGGSGSAITTGYRFTNFEPFTVNSVNDSLEPVGGLYE
IQIPSEFTIGNMEEFIQTSSPKVTIDCAAFVCGDYAACKLQLVEYGSFCDNINAILTEVNEL
LDTTQLQVANSLMNGVTLSTKLKDGVNFNVDDINFSPVLGCLGSECSKASSRSAIEDLLFDK
VKLSDVGFVEAYNNCTGGAEIRDLICVQSYKGIKVLPPLLSENQISGYTLAATSASLFPPWT
AAAGVPFYLNVQYRINGLGVTMDVLSQNQKLIANAFNNALHAIQQGFDATNSALVKIQAVVN
ANAEALNNLLQQLSNRFGAISASLQEILSRLDPPEAEAQIDRLINGRLTALNAYVSQQLSDS
TLVKFSAAQAMEKVNECVKSQSSRINFCGNGNHIISLVQNAPYGLYFIHFNYVPTKYVTAKV
SPGLCIAGNRGIAPKSGYFVNVNNTWMYTGSGYYYPEPITENNVVVMSTCAVNYTKAPYVML
NTSIPNLPDFKEELDQWFKNQTSVAPDLSLDYINVTFLDLQVEMNRLQEAIKVLNHSYINLK
DIGTYEYYVKWPWYVWLLICLAGVAMLVLLFFICCSNGSLQCRICI
PDI-OC43-H1cCT-AA
(SEQ ID NO: 146)
MAKNVAIFGLLFSLLVLVPSQIFAVIGDLNCTLDPRLKGSFNNRDTGPPSISIDTVDVTNGL
GTYYVLDRVYLNTTLFLNGYYPTSGSTYRNMALKGTDLLSTLWFKPPFLSDFINGIFAKVKN
TKVFKDGVMYSEFPAITIGSTFVNTSYSVVVQPRTINSTQDGVNKLQGLLEVSVCQYNMCEY
PHTICHPNLGNHFKELWHYDTGVVSCLYKRNFTYDVNATYLYFHFYQEGGTFYAYFTDTGFV
TKFLFNVYLGMALSHYYVMPLTCIRRPKDGFSLEYWVTPLTPRQYLLAFNQDGIIFNAVDCM
SDFMSEIKCKTQSIAPPTGVYELNGYTVQPVADVYRRKPDLPNCNIEAWLNDKSVPSPLNWE
RKTFSNCNFNMSSLMSFIQADSFTCNNIDAAKIYGMCFSSITIDKFAIPNRRKVDLQLGNLG
YLQSSNYRIDTTATSCQLYYNLPAANVSVSRFNPSTWNKRFGFIEDSVFVPQPTGVFTNHSV
VYAQHCFKAPKNFCPCSSCSCPGKNNGIGTCPAGTNSLTCDNLCTLDPITLKAPDTYKCPQS
KSLVGIGEHCSGLAVKSDYCGNNSCTCQPQAFLGWSADSCLQGDKCNIFANFILHDVNNGLT
CSTDLQKANTEIELGVCVNYDLYGISGQGIFVEVNATYYNSWONLLYDSNGNLYGFRDYITN
RTFMIHSCYSGRVSAAYHANSSEPALLFRNIKCNYVFNNSLTRQLQPINYSFDSYLGCVVNA
YNSTAISVQTCDLTVGSGYCVDYSKNGGSGSAITTGYRFTNFEPFTVNSVNDSLEPVGGLYE
IQIPSEFTIGNMEEFIQTSSPKVTIDCAAFVCGDYAACKLQLVEYGSFCDNINAILTEVNEL
LDTTQLQVANSLMNGVTLSTKLKDGVNFNVDDINFSPVLGCLGSECSKASSRSAIEDLLFDK
VKLSDVGFVEAYNNCTGGAEIRDLICVQSYKGIKVLPPLLSENQISGYTLAATSASLFPPWT
AAAGVPFYLNVQYRINGLGVTMDVLSQNQKLIANAFNNALHAIQQGFDATNSALVKIQAVVN
ANAEALNNLLQQLSNRFGAISASLQEILSRLDPPEAEAQIDRLINGRLTALNAYVSQQLSDS
TLVKFSAAQAMEKVNECVKSQSSRINFCGNGNHIISLVQNAPYGLYFIHFNYVPTKYVTAKV
SPGLCIAGNRGIAPKSGYFVNVNNTWMYTGSGYYYPEPITENNVVVMSTCAVNYTKAPYVML
NTSIPNLPDFKEELDQWFKNQTSVAPDLSLDYINVTFLDLQVEMNRLQEAIKVLNHSYINLK
DIGTYEYYVKWPWYVWLLICLAGVAMLVLLFFISFWMCSNGSLQCRICI
IF(CoV229EwtCT).r
(SEQ ID NO: 147)
ACGACACGACTAAGGCCTTCACTGTATGTGGATCTTTTCGACATCGTA
PDI-229E -wtTMCT-DNA
(SEQ ID NO: 148)
atggcgaaaaacgttgcgattttcggcttattgttttctcttcttgtgttggttccttctca
gatcttcgcgCAAACGACTAATGGGCTGAACACCAGTTACAGCGTCTGTAACGGCTGCGTCG
GATATAGCGAGAACGTGTTCGCAGTGGAAAGTGGGGGGTACATTCCTTCCGACTTCGCTTTC
AATAACTGGTTTCTCCTGACTAACACAAGCTCCGTCGTGGATGGCGTGGTCAGGTCCTTTCA
GCCTCTTCTCCTGAATTGCCTGTGGTCTGTGTCCGGGTTAAGATTCACTACAGGCTTCGTAT
ACTTCAACGGGACGGGCCGGGGGGATTGCAAGGGCTTCTCCTCCGACGTGCTGTCAGATGTG
ATCCGTTACAATCTGAACTTCGAAGAGAACTTACGGCGGGGGACAATCCTGTTCAAAACATC
ATATGGCGTAGTCGTATTTTACTGCACCAATAATACCCTGGTGAGTGGGGACGCCCATATTC
CCTTCGGAACAGTGCTGGGTAACTTTTACTGTTTTGTCAACACTACGATCGGAAACGAAACC
ACTAGCGCCTTTGTCGGAGCTCTGCCAAAAACAGTTAGGGAGTTCGTGATCTCTCGGACCGG
TCACTTCTATATCAACGGCTACCGTTATTTTACTTTGGGCAACGTCGAAGCCGTCAATTTTA
ATGTGACAACTGCAGAGACAACTGACTTTTGCACTGTGGCTCTCGCCAGTTATGCCGATGTG
CTGGTGAATGTAAGTCAAACGTCAATTGCCAACATCATCTATTGTAACTCAGTAATCAACCG
GCTCCGCTGTGACCAACTCTCATTCGACGTCCCCGACGGATTCTATTCCACGAGCCCGATTC
AGAGCGTGGAACTGCCAGTTTCCATCGTATCCCTCCCAGTTTACCACAAGCACACTTTTATC
GTTCTCTACGTAGATTTTAAACCCCAGTCAGGAGGAGGGAAATGCTTCAACTGCTACCCGGC
TGGCGTGAACATCACCTTGGCCAATTTTAATGAAACTAAAGGGCCCCTTTGCGTGGATACGT
CACACTTTACCACAAAGTATGTTGCAGTCTATGCTAACGTCGGCAGGTGGTCAGCGTCCATT
AACACAGGCAATTGCCCGTTCTCTTTCGGGAAAGTGAACAACTTCGTGAAGTTTGGAAGTGT
GTGCTTCAGTTTGAAAGACATTCCGGGCGGCTGCGCCATGCCTATTGTGGCTAATTGGGCTT
ATTCCAAGTACTACACCATTGGCTCTCTCTACGTTAGCTGGAGCGACGGTGACGGTATAACG
GGCGTACCACAACCGGTGGAAGGGGTCAGCTCTTTCATGAATGTCACTCTGGACAAGTGTAC
CAAATATAATATATACGATGTGAGTGGAGTGGGCGTTATACGCGTGTCTAACGACACCTTTC
TAAACGGCATAACCTACACAAGCACGTCAGGCAATCTGTTAGGTTTTAAAGACGTCACTAAA
GGCACTATATATAGCATCACCCCATGCAACCCACCTGATCAATTAGTCGTATATCAGCAAGC
TGTTGTGGGTGCTATGCTGTCAGAAAACTTCACCAGCTACGGGTTCTCCAATGTGGTGGAAC
TGCCCAAATTCTTTTACGCTAGCAATGGCACATATAACTGTACTGACGCCGTCTTGACTTAC
AGTTCATTCGGAGTGTGCGCGGACGGCAGCATTATCGCCGTGCAGCCGGCCAATGTCAGCTA
TGATTCCGTTTCCGCCATCGTGACAGCCAACTTGTCGATTCCCTCTAACTGGACAACGTCTG
TCCAAGTCGAATATCTGCAGATCACCTCAACCCCCATAGTAGTCGATTGCTCAACCTACGTC
TGCAACGGTAATGTCAGATGTGTCGAGCTGCTCAAGCAGTACACCTCCGCCTGTAAGACTAT
TGAGGATGCATTAAGAAATAGTGCAAGATTGGAAAGCGCCGATGTGTCGGAAATGCTAACCT
TCGATAAGAAGGCATTCACACTGGCGAACGTAAGCTCTTTCGGCGATTACAACCTGTCTTCG
GTAATCCCTAGCTTGCCCACATCCGGCTCTCGGGTGGCGGGGCGGAGCGCTATCGAGGACAT
TTTATTCTCGAAACTGGTTACATCTGGGCTCGGAACTGTGGACGCCGATTACAAGAAGTGCA
CCAAGGGCCTAAGCATCGCCGACCTCGCCTGTGCTCAGTACTACAACGGAATTATGGTGCTG
CCAGGTGTCGCTGACGCAGAGCGGATGGCTATGTATACCGGCAGTCTCATTGGCGGGATTGC
GTTGGGCGGCCTGACGTCCGCTGTCTCCATCCCTTTCTCTCTGGCTATACAAGCCCGACTGA
ATTATGTGGCCCTGCAGACTGATGTCCTGCAAGAAAATCAGAAGATTCTTGCCGCCAGCTTC
AACAAGGCCATGACTAATATTGTGGATGCGTTTACCGGAGTGAATGACGCCATCACCCAAAC
GTCCCAAGCCCTGCAGACAGTCGCCACGGCGTTAAACAAAATCCAGGATGTAGTGAATCAGC
AAGGGAACAGCTTGAATCACCTGACGTCCCAGTTAAGACAGAACTTTCAGGCAATCAGTAGC
TCAATCCAGGCTATCTACGATCGATTAGATCCTCCTCAGGCAGATCAGCAGGTGGATCGGCT
CATCACCGGCCGCCTCGCGGCATTGAATGTTTTCGTAAGTCATACCTTGACCAAGTACACGG
AGGTGAGGGCCAGTCGCCAGCTGGCTCAGCAAAAAGTGAATGAGTGTGTGAAATCACAGAGC
AAACGGTACGGGTTTTGTGGAAATGGGACGCACATCTTTAGCATCGTTAATGCTGCCCCCGA
AGGGTTAGTCTTCCTGCACACTGTGCTCCTTCCTACCCAGTATAAAGATGTCGAAGCATGGT
CTGGGCTCTGTGTCGATGGAACTAACGGTTATGTCCTTCGACAGCCAAACCTCGCTCTCTAT
AAAGAAGGGAATTACTATAGGATCACCTCAAGAATCATGTTCGAGCCCAGGATACCAACAAT
GGCCGATTTTGTGCAGATTGAAAATTGTAACGTGACCTTTGTGAATATCAGTCGATCCGAGC
TTCAAACGATTGTTCCTGAGTACATCGACGTGAATAAAACTCTACAAGAGCTGTCCTATAAA
CTGCCTAATTATACCGTGCCTGACCTTGTAGTCGAGCAATACAACCAGACTATTCTGAACCT
GACATCGGAAATCTCTACATTGGAGAATAAAAGCGCCGAGCTCAATTACACAGTGCAGAAGC
TGCAGACCCTGATCGACAATATTAACAGCACTCTTGTGGACTTAAAGTGGCTGAACCGTGTG
GAGACTTACATCAAGTGGCCCTGGTGGGTGTGGCTCTGTATTTCCGTGGTCCTTATATTTGT
TGTAAGTATGCTGCTCCTGTGCTGTTGCTCAACCGGGTGCTGCGGTTTTTTCTCCTGTTTCG
CCTCATCCATCCGTGGCTGTTGTGAGAGCACTAAACTGCCATATTACGATGTCGAAAAGATC
CACATACAGTGA
PDI-229E -H5iTMCT-DNA
(SEQ ID NO: 149)
atggcgaaaaacgttgcgattttcggcttattgttttctcttcttgtgttggttccttctca
gatcttcgcgCAAACGACTAATGGGCTGAACACCAGTTACAGCGTCTGTAACGGCTGCGTCG
GATATAGCGAGAACGTGTTCGCAGTGGAAAGTGGGGGGTACATTCCTTCCGACTTCGCTTTC
AATAACTGGTTTCTCCTGACTAACACAAGCTCCGTCGTGGATGGCGTGGTCAGGTCCTTTCA
GCCTCTTCTCCTGAATTGCCTGTGGTCTGTGTCCGGGTTAAGATTCACTACAGGCTTCGTAT
ACTTCAACGGGACGGGCCGGGGGGATTGCAAGGGCTTCTCCTCCGACGTGCTGTCAGATGTG
ATCCGTTACAATCTGAACTTCGAAGAGAACTTACGGCGGGGGACAATCCTGTTCAAAACATC
ATATGGCGTAGTCGTATTTTACTGCACCAATAATACCCTGGTGAGTGGGGACGCCCATATTC
CCTTCGGAACAGTGCTGGGTAACTTTTACTGTTTTGTCAACACTACGATCGGAAACGAAACC
ACTAGCGCCTTTGTCGGAGCTCTGCCAAAAACAGTTAGGGAGTTCGTGATCTCTCGGACCGG
TCACTTCTATATCAACGGCTACCGTTATTTTACTTTGGGCAACGTCGAAGCCGTCAATTTTA
ATGTGACAACTGCAGAGACAACTGACTTTTGCACTGTGGCTCTCGCCAGTTATGCCGATGTG
CTGGTGAATGTAAGTCAAACGTCAATTGCCAACATCATCTATTGTAACTCAGTAATCAACCG
GCTCCGCTGTGACCAACTCTCATTCGACGTCCCCGACGGATTCTATTCCACGAGCCCGATTC
AGAGCGTGGAACTGCCAGTTTCCATCGTATCCCTCCCAGTTTACCACAAGCACACTTTTATC
GTTCTCTACGTAGATTTTAAACCCCAGTCAGGAGGAGGGAAATGCTTCAACTGCTACCCGGC
TGGCGTGAACATCACCTTGGCCAATTTTAATGAAACTAAAGGGCCCCTTTGCGTGGATACGT
CACACTTTACCACAAAGTATGTTGCAGTCTATGCTAACGTCGGCAGGTGGTCAGCGTCCATT
AACACAGGCAATTGCCCGTTCTCTTTCGGGAAAGTGAACAACTTCGTGAAGTTTGGAAGTGT
GTGCTTCAGTTTGAAAGACATTCCGGGCGGCTGCGCCATGCCTATTGTGGCTAATTGGGCTT
ATTCCAAGTACTACACCATTGGCTCTCTCTACGTTAGCTGGAGCGACGGTGACGGTATAACG
GGCGTACCACAACCGGTGGAAGGGGTCAGCTCTTTCATGAATGTCACTCTGGACAAGTGTAC
CAAATATAATATATACGATGTGAGTGGAGTGGGCGTTATACGCGTGTCTAACGACACCTTTC
TAAACGGCATAACCTACACAAGCACGTCAGGCAATCTGTTAGGTTTTAAAGACGTCACTAAA
GGCACTATATATAGCATCACCCCATGCAACCCACCTGATCAATTAGTCGTATATCAGCAAGC
TGTTGTGGGTGCTATGCTGTCAGAAAACTTCACCAGCTACGGGTTCTCCAATGTGGTGGAAC
TGCCCAAATTCTTTTACGCTAGCAATGGCACATATAACTGTACTGACGCCGTCTTGACTTAC
AGTTCATTCGGAGTGTGCGCGGACGGCAGCATTATCGCCGTGCAGCCGGCCAATGTCAGCTA
TGATTCCGTTTCCGCCATCGTGACAGCCAACTTGTCGATTCCCTCTAACTGGACAACGTCTG
TCCAAGTCGAATATCTGCAGATCACCTCAACCCCCATAGTAGTCGATTGCTCAACCTACGTC
TGCAACGGTAATGTCAGATGTGTCGAGCTGCTCAAGCAGTACACCTCCGCCTGTAAGACTAT
TGAGGATGCATTAAGAAATAGTGCAAGATTGGAAAGCGCCGATGTGTCGGAAATGCTAACCT
TCGATAAGAAGGCATTCACACTGGCGAACGTAAGCTCTTTCGGCGATTACAACCTGTCTTCG
GTAATCCCTAGCTTGCCCACATCCGGCTCTCGGGTGGCGGGGCGGAGCGCTATCGAGGACAT
TTTATTCTCGAAACTGGTTACATCTGGGCTCGGAACTGTGGACGCCGATTACAAGAAGTGCA
CCAAGGGCCTAAGCATCGCCGACCTCGCCTGTGCTCAGTACTACAACGGAATTATGGTGCTG
CCAGGTGTCGCTGACGCAGAGCGGATGGCTATGTATACCGGCAGTCTCATTGGCGGGATTGC
GTTGGGCGGCCTGACGTCCGCTGTCTCCATCCCTTTCTCTCTGGCTATACAAGCCCGACTGA
ATTATGTGGCCCTGCAGACTGATGTCCTGCAAGAAAATCAGAAGATTCTTGCCGCCAGCTTC
AACAAGGCCATGACTAATATTGTGGATGCGTTTACCGGAGTGAATGACGCCATCACCCAAAC
GTCCCAAGCCCTGCAGACAGTCGCCACGGCGTTAAACAAAATCCAGGATGTAGTGAATCAGC
AAGGGAACAGCTTGAATCACCTGACGTCCCAGTTAAGACAGAACTTTCAGGCAATCAGTAGC
TCAATCCAGGCTATCTACGATCGATTAGATCCTCCTCAGGCAGATCAGCAGGTGGATCGGCT
CATCACCGGCCGCCTCGCGGCATTGAATGTTTTCGTAAGTCATACCTTGACCAAGTACACGG
AGGTGAGGGCCAGTCGCCAGCTGGCTCAGCAAAAAGTGAATGAGTGTGTGAAATCACAGAGC
AAACGGTACGGGTTTTGTGGAAATGGGACGCACATCTTTAGCATCGTTAATGCTGCCCCCGA
AGGGTTAGTCTTCCTGCACACTGTGCTCCTTCCTACCCAGTATAAAGATGTCGAAGCATGGT
CTGGGCTCTGTGTCGATGGAACTAACGGTTATGTCCTTCGACAGCCAAACCTCGCTCTCTAT
AAAGAAGGGAATTACTATAGGATCACCTCAAGAATCATGTTCGAGCCCAGGATACCAACAAT
GGCCGATTTTGTGCAGATTGAAAATTGTAACGTGACCTTTGTGAATATCAGTCGATCCGAGC
TTCAAACGATTGTTCCTGAGTACATCGACGTGAATAAAACTCTACAAGAGCTGTCCTATAAA
CTGCCTAATTATACCGTGCCTGACCTTGTAGTCGAGCAATACAACCAGACTATTCTGAACCT
GACATCGGAAATCTCTACATTGGAGAATAAAAGCGCCGAGCTCAATTACACAGTGCAGAAGC
TGCAGACCCTGATCGACAATATTAACAGCACTCTTGTGGACTTAAAGTGGCTGAACCGTGTG
GAGACTTACATCAAGTGGCCCTGGTGGcaaatactgtcaatttattcaacagtggcgagttc
cctagcactggcaatcatgatggctggtctatctttatggatgtgctccaatggatcgttac
aatgcagaatttgcattTGA
PDI-229E -H5iCT-DNA
(SEQ ID NO: 150)
atggcgaaaaacgttgcgattttcggcttattgttttctcttcttgtgttggttccttctca
gatcttcgcgCAAACGACTAATGGGCTGAACACCAGTTACAGCGTCTGTAACGGCTGCGTCG
GATATAGCGAGAACGTGTTCGCAGTGGAAAGTGGGGGGTACATTCCTTCCGACTTCGCTTTC
AATAACTGGTTTCTCCTGACTAACACAAGCTCCGTCGTGGATGGCGTGGTCAGGTCCTTTCA
GCCTCTTCTCCTGAATTGCCTGTGGTCTGTGTCCGGGTTAAGATTCACTACAGGCTTCGTAT
ACTTCAACGGGACGGGCCGGGGGGATTGCAAGGGCTTCTCCTCCGACGTGCTGTCAGATGTG
ATCCGTTACAATCTGAACTTCGAAGAGAACTTACGGCGGGGGACAATCCTGTTCAAAACATC
ATATGGCGTAGTCGTATTTTACTGCACCAATAATACCCTGGTGAGTGGGGACGCCCATATTC
CCTTCGGAACAGTGCTGGGTAACTTTTACTGTTTTGTCAACACTACGATCGGAAACGAAACC
ACTAGCGCCTTTGTCGGAGCTCTGCCAAAAACAGTTAGGGAGTTCGTGATCTCTCGGACCGG
TCACTTCTATATCAACGGCTACCGTTATTTTACTTTGGGCAACGTCGAAGCCGTCAATTTTA
ATGTGACAACTGCAGAGACAACTGACTTTTGCACTGTGGCTCTCGCCAGTTATGCCGATGTG
CTGGTGAATGTAAGTCAAACGTCAATTGCCAACATCATCTATTGTAACTCAGTAATCAACCG
GCTCCGCTGTGACCAACTCTCATTCGACGTCCCCGACGGATTCTATTCCACGAGCCCGATTC
AGAGCGTGGAACTGCCAGTTTCCATCGTATCCCTCCCAGTTTACCACAAGCACACTTTTATC
GTTCTCTACGTAGATTTTAAACCCCAGTCAGGAGGAGGGAAATGCTTCAACTGCTACCCGGC
TGGCGTGAACATCACCTTGGCCAATTTTAATGAAACTAAAGGGCCCCTTTGCGTGGATACGT
CACACTTTACCACAAAGTATGTTGCAGTCTATGCTAACGTCGGCAGGTGGTCAGCGTCCATT
AACACAGGCAATTGCCCGTTCTCTTTCGGGAAAGTGAACAACTTCGTGAAGTTTGGAAGTGT
GTGCTTCAGTTTGAAAGACATTCCGGGCGGCTGCGCCATGCCTATTGTGGCTAATTGGGCTT
ATTCCAAGTACTACACCATTGGCTCTCTCTACGTTAGCTGGAGCGACGGTGACGGTATAACG
GGCGTACCACAACCGGTGGAAGGGGTCAGCTCTTTCATGAATGTCACTCTGGACAAGTGTAC
CAAATATAATATATACGATGTGAGTGGAGTGGGCGTTATACGCGTGTCTAACGACACCTTTC
TAAACGGCATAACCTACACAAGCACGTCAGGCAATCTGTTAGGTTTTAAAGACGTCACTAAA
GGCACTATATATAGCATCACCCCATGCAACCCACCTGATCAATTAGTCGTATATCAGCAAGC
TGTTGTGGGTGCTATGCTGTCAGAAAACTTCACCAGCTACGGGTTCTCCAATGTGGTGGAAC
TGCCCAAATTCTTTTACGCTAGCAATGGCACATATAACTGTACTGACGCCGTCTTGACTTAC
AGTTCATTCGGAGTGTGCGCGGACGGCAGCATTATCGCCGTGCAGCCGGCCAATGTCAGCTA
TGATTCCGTTTCCGCCATCGTGACAGCCAACTTGTCGATTCCCTCTAACTGGACAACGTCTG
TCCAAGTCGAATATCTGCAGATCACCTCAACCCCCATAGTAGTCGATTGCTCAACCTACGTC
TGCAACGGTAATGTCAGATGTGTCGAGCTGCTCAAGCAGTACACCTCCGCCTGTAAGACTAT
TGAGGATGCATTAAGAAATAGTGCAAGATTGGAAAGCGCCGATGTGTCGGAAATGCTAACCT
TCGATAAGAAGGCATTCACACTGGCGAACGTAAGCTCTTTCGGCGATTACAACCTGTCTTCG
GTAATCCCTAGCTTGCCCACATCCGGCTCTCGGGTGGCGGGGCGGAGCGCTATCGAGGACAT
TTTATTCTCGAAACTGGTTACATCTGGGCTCGGAACTGTGGACGCCGATTACAAGAAGTGCA
CCAAGGGCCTAAGCATCGCCGACCTCGCCTGTGCTCAGTACTACAACGGAATTATGGTGCTG
CCAGGTGTCGCTGACGCAGAGCGGATGGCTATGTATACCGGCAGTCTCATTGGCGGGATTGC
GTTGGGCGGCCTGACGTCCGCTGTCTCCATCCCTTTCTCTCTGGCTATACAAGCCCGACTGA
ATTATGTGGCCCTGCAGACTGATGTCCTGCAAGAAAATCAGAAGATTCTTGCCGCCAGCTTC
AACAAGGCCATGACTAATATTGTGGATGCGTTTACCGGAGTGAATGACGCCATCACCCAAAC
GTCCCAAGCCCTGCAGACAGTCGCCACGGCGTTAAACAAAATCCAGGATGTAGTGAATCAGC
AAGGGAACAGCTTGAATCACCTGACGTCCCAGTTAAGACAGAACTTTCAGGCAATCAGTAGC
TCAATCCAGGCTATCTACGATCGATTAGATCCTCCTCAGGCAGATCAGCAGGTGGATCGGCT
CATCACCGGCCGCCTCGCGGCATTGAATGTTTTCGTAAGTCATACCTTGACCAAGTACACGG
AGGTGAGGGCCAGTCGCCAGCTGGCTCAGCAAAAAGTGAATGAGTGTGTGAAATCACAGAGC
AAACGGTACGGGTTTTGTGGAAATGGGACGCACATCTTTAGCATCGTTAATGCTGCCCCCGA
AGGGTTAGTCTTCCTGCACACTGTGCTCCTTCCTACCCAGTATAAAGATGTCGAAGCATGGT
CTGGGCTCTGTGTCGATGGAACTAACGGTTATGTCCTTCGACAGCCAAACCTCGCTCTCTAT
AAAGAAGGGAATTACTATAGGATCACCTCAAGAATCATGTTCGAGCCCAGGATACCAACAAT
GGCCGATTTTGTGCAGATTGAAAATTGTAACGTGACCTTTGTGAATATCAGTCGATCCGAGC
TTCAAACGATTGTTCCTGAGTACATCGACGTGAATAAAACTCTACAAGAGCTGTCCTATAAA
CTGCCTAATTATACCGTGCCTGACCTTGTAGTCGAGCAATACAACCAGACTATTCTGAACCT
GACATCGGAAATCTCTACATTGGAGAATAAAAGCGCCGAGCTCAATTACACAGTGCAGAAGC
TGCAGACCCTGATCGACAATATTAACAGCACTCTTGTGGACTTAAAGTGGCTGAACCGTGTG
GAGACTTACATCAAGTGGCCCTGGTGGGTGTGGCTCTGTATTTCCGTGGTCCTTATATTTGT
TGTAAGTATGCTGCTCCTGtctttatggatgtgctccaatggatcgttacaatgcagaattt
gcattTGA
PDI-229E -H5iCT(V4)-DNA
(SEQ ID NO: 151)
atggcgaaaaacgttgcgattttcggcttattgttttctcttcttgtgttggttccttctca
gatcttcgcgCAAACGACTAATGGGCTGAACACCAGTTACAGCGTCTGTAACGGCTGCGTCG
GATATAGCGAGAACGTGTTCGCAGTGGAAAGTGGGGGGTACATTCCTTCCGACTTCGCTTTC
AATAACTGGTTTCTCCTGACTAACACAAGCTCCGTCGTGGATGGCGTGGTCAGGTCCTTTCA
GCCTCTTCTCCTGAATTGCCTGTGGTCTGTGTCCGGGTTAAGATTCACTACAGGCTTCGTAT
ACTTCAACGGGACGGGCCGGGGGGATTGCAAGGGCTTCTCCTCCGACGTGCTGTCAGATGTG
ATCCGTTACAATCTGAACTTCGAAGAGAACTTACGGCGGGGGACAATCCTGTTCAAAACATC
ATATGGCGTAGTCGTATTTTACTGCACCAATAATACCCTGGTGAGTGGGGACGCCCATATTC
CCTTCGGAACAGTGCTGGGTAACTTTTACTGTTTTGTCAACACTACGATCGGAAACGAAACC
ACTAGCGCCTTTGTCGGAGCTCTGCCAAAAACAGTTAGGGAGTTCGTGATCTCTCGGACCGG
TCACTTCTATATCAACGGCTACCGTTATTTTACTTTGGGCAACGTCGAAGCCGTCAATTTTA
ATGTGACAACTGCAGAGACAACTGACTTTTGCACTGTGGCTCTCGCCAGTTATGCCGATGTG
CTGGTGAATGTAAGTCAAACGTCAATTGCCAACATCATCTATTGTAACTCAGTAATCAACCG
GCTCCGCTGTGACCAACTCTCATTCGACGTCCCCGACGGATTCTATTCCACGAGCCCGATTC
AGAGCGTGGAACTGCCAGTTTCCATCGTATCCCTCCCAGTTTACCACAAGCACACTTTTATC
GTTCTCTACGTAGATTTTAAACCCCAGTCAGGAGGAGGGAAATGCTTCAACTGCTACCCGGC
TGGCGTGAACATCACCTTGGCCAATTTTAATGAAACTAAAGGGCCCCTTTGCGTGGATACGT
CACACTTTACCACAAAGTATGTTGCAGTCTATGCTAACGTCGGCAGGTGGTCAGCGTCCATT
AACACAGGCAATTGCCCGTTCTCTTTCGGGAAAGTGAACAACTTCGTGAAGTTTGGAAGTGT
GTGCTTCAGTTTGAAAGACATTCCGGGCGGCTGCGCCATGCCTATTGTGGCTAATTGGGCTT
ATTCCAAGTACTACACCATTGGCTCTCTCTACGTTAGCTGGAGCGACGGTGACGGTATAACG
GGCGTACCACAACCGGTGGAAGGGGTCAGCTCTTTCATGAATGTCACTCTGGACAAGTGTAC
CAAATATAATATATACGATGTGAGTGGAGTGGGCGTTATACGCGTGTCTAACGACACCTTTC
TAAACGGCATAACCTACACAAGCACGTCAGGCAATCTGTTAGGTTTTAAAGACGTCACTAAA
GGCACTATATATAGCATCACCCCATGCAACCCACCTGATCAATTAGTCGTATATCAGCAAGC
TGTTGTGGGTGCTATGCTGTCAGAAAACTTCACCAGCTACGGGTTCTCCAATGTGGTGGAAC
TGCCCAAATTCTTTTACGCTAGCAATGGCACATATAACTGTACTGACGCCGTCTTGACTTAC
AGTTCATTCGGAGTGTGCGCGGACGGCAGCATTATCGCCGTGCAGCCGGCCAATGTCAGCTA
TGATTCCGTTTCCGCCATCGTGACAGCCAACTTGTCGATTCCCTCTAACTGGACAACGTCTG
TCCAAGTCGAATATCTGCAGATCACCTCAACCCCCATAGTAGTCGATTGCTCAACCTACGTC
TGCAACGGTAATGTCAGATGTGTCGAGCTGCTCAAGCAGTACACCTCCGCCTGTAAGACTAT
TGAGGATGCATTAAGAAATAGTGCAAGATTGGAAAGCGCCGATGTGTCGGAAATGCTAACCT
TCGATAAGAAGGCATTCACACTGGCGAACGTAAGCTCTTTCGGCGATTACAACCTGTCTTCG
GTAATCCCTAGCTTGCCCACATCCGGCTCTCGGGTGGCGGGGCGGAGCGCTATCGAGGACAT
TTTATTCTCGAAACTGGTTACATCTGGGCTCGGAACTGTGGACGCCGATTACAAGAAGTGCA
CCAAGGGCCTAAGCATCGCCGACCTCGCCTGTGCTCAGTACTACAACGGAATTATGGTGCTG
CCAGGTGTCGCTGACGCAGAGCGGATGGCTATGTATACCGGCAGTCTCATTGGCGGGATTGC
GTTGGGCGGCCTGACGTCCGCTGTCTCCATCCCTTTCTCTCTGGCTATACAAGCCCGACTGA
ATTATGTGGCCCTGCAGACTGATGTCCTGCAAGAAAATCAGAAGATTCTTGCCGCCAGCTTC
AACAAGGCCATGACTAATATTGTGGATGCGTTTACCGGAGTGAATGACGCCATCACCCAAAC
GTCCCAAGCCCTGCAGACAGTCGCCACGGCGTTAAACAAAATCCAGGATGTAGTGAATCAGC
AAGGGAACAGCTTGAATCACCTGACGTCCCAGTTAAGACAGAACTTTCAGGCAATCAGTAGC
TCAATCCAGGCTATCTACGATCGATTAGATCCTCCTCAGGCAGATCAGCAGGTGGATCGGCT
CATCACCGGCCGCCTCGCGGCATTGAATGTTTTCGTAAGTCATACCTTGACCAAGTACACGG
AGGTGAGGGCCAGTCGCCAGCTGGCTCAGCAAAAAGTGAATGAGTGTGTGAAATCACAGAGC
AAACGGTACGGGTTTTGTGGAAATGGGACGCACATCTTTAGCATCGTTAATGCTGCCCCCGA
AGGGTTAGTCTTCCTGCACACTGTGCTCCTTCCTACCCAGTATAAAGATGTCGAAGCATGGT
CTGGGCTCTGTGTCGATGGAACTAACGGTTATGTCCTTCGACAGCCAAACCTCGCTCTCTAT
AAAGAAGGGAATTACTATAGGATCACCTCAAGAATCATGTTCGAGCCCAGGATACCAACAAT
GGCCGATTTTGTGCAGATTGAAAATTGTAACGTGACCTTTGTGAATATCAGTCGATCCGAGC
TTCAAACGATTGTTCCTGAGTACATCGACGTGAATAAAACTCTACAAGAGCTGTCCTATAAA
CTGCCTAATTATACCGTGCCTGACCTTGTAGTCGAGCAATACAACCAGACTATTCTGAACCT
GACATCGGAAATCTCTACATTGGAGAATAAAAGCGCCGAGCTCAATTACACAGTGCAGAAGC
TGCAGACCCTGATCGACAATATTAACAGCACTCTTGTGGACTTAAAGTGGCTGAACCGTGTG
GAGACTTACATCAAGTGGCCCTGGTGGGTGTGGCTCTGTATTTCCGTGGTCCTTATATTTGT
TGTAAGTATGCTGCTCCTGtgctgctccaatggatcgttacaatgcagaatttgcattTGA
PDI-229E -H1cCT-DNA
(SEQ ID NO: 152)
atggcgaaaaacgttgcgattttcggcttattgttttctcttcttgtgttggttccttctca
gatcttcgcgCAAACGACTAATGGGCTGAACACCAGTTACAGCGTCTGTAACGGCTGCGTCG
GATATAGCGAGAACGTGTTCGCAGTGGAAAGTGGGGGGTACATTCCTTCCGACTTCGCTTTC
AATAACTGGTTTCTCCTGACTAACACAAGCTCCGTCGTGGATGGCGTGGTCAGGTCCTTTCA
GCCTCTTCTCCTGAATTGCCTGTGGTCTGTGTCCGGGTTAAGATTCACTACAGGCTTCGTAT
ACTTCAACGGGACGGGCCGGGGGGATTGCAAGGGCTTCTCCTCCGACGTGCTGTCAGATGTG
ATCCGTTACAATCTGAACTTCGAAGAGAACTTACGGCGGGGGACAATCCTGTTCAAAACATC
ATATGGCGTAGTCGTATTTTACTGCACCAATAATACCCTGGTGAGTGGGGACGCCCATATTC
CCTTCGGAACAGTGCTGGGTAACTTTTACTGTTTTGTCAACACTACGATCGGAAACGAAACC
ACTAGCGCCTTTGTCGGAGCTCTGCCAAAAACAGTTAGGGAGTTCGTGATCTCTCGGACCGG
TCACTTCTATATCAACGGCTACCGTTATTTTACTTTGGGCAACGTCGAAGCCGTCAATTTTA
ATGTGACAACTGCAGAGACAACTGACTTTTGCACTGTGGCTCTCGCCAGTTATGCCGATGTG
CTGGTGAATGTAAGTCAAACGTCAATTGCCAACATCATCTATTGTAACTCAGTAATCAACCG
GCTCCGCTGTGACCAACTCTCATTCGACGTCCCCGACGGATTCTATTCCACGAGCCCGATTC
AGAGCGTGGAACTGCCAGTTTCCATCGTATCCCTCCCAGTTTACCACAAGCACACTTTTATC
GTTCTCTACGTAGATTTTAAACCCCAGTCAGGAGGAGGGAAATGCTTCAACTGCTACCCGGC
TGGCGTGAACATCACCTTGGCCAATTTTAATGAAACTAAAGGGCCCCTTTGCGTGGATACGT
CACACTTTACCACAAAGTATGTTGCAGTCTATGCTAACGTCGGCAGGTGGTCAGCGTCCATT
AACACAGGCAATTGCCCGTTCTCTTTCGGGAAAGTGAACAACTTCGTGAAGTTTGGAAGTGT
GTGCTTCAGTTTGAAAGACATTCCGGGCGGCTGCGCCATGCCTATTGTGGCTAATTGGGCTT
ATTCCAAGTACTACACCATTGGCTCTCTCTACGTTAGCTGGAGCGACGGTGACGGTATAACG
GGCGTACCACAACCGGTGGAAGGGGTCAGCTCTTTCATGAATGTCACTCTGGACAAGTGTAC
CAAATATAATATATACGATGTGAGTGGAGTGGGCGTTATACGCGTGTCTAACGACACCTTTC
TAAACGGCATAACCTACACAAGCACGTCAGGCAATCTGTTAGGTTTTAAAGACGTCACTAAA
GGCACTATATATAGCATCACCCCATGCAACCCACCTGATCAATTAGTCGTATATCAGCAAGC
TGTTGTGGGTGCTATGCTGTCAGAAAACTTCACCAGCTACGGGTTCTCCAATGTGGTGGAAC
TGCCCAAATTCTTTTACGCTAGCAATGGCACATATAACTGTACTGACGCCGTCTTGACTTAC
AGTTCATTCGGAGTGTGCGCGGACGGCAGCATTATCGCCGTGCAGCCGGCCAATGTCAGCTA
TGATTCCGTTTCCGCCATCGTGACAGCCAACTTGTCGATTCCCTCTAACTGGACAACGTCTG
TCCAAGTCGAATATCTGCAGATCACCTCAACCCCCATAGTAGTCGATTGCTCAACCTACGTC
TGCAACGGTAATGTCAGATGTGTCGAGCTGCTCAAGCAGTACACCTCCGCCTGTAAGACTAT
TGAGGATGCATTAAGAAATAGTGCAAGATTGGAAAGCGCCGATGTGTCGGAAATGCTAACCT
TCGATAAGAAGGCATTCACACTGGCGAACGTAAGCTCTTTCGGCGATTACAACCTGTCTTCG
GTAATCCCTAGCTTGCCCACATCCGGCTCTCGGGTGGCGGGGCGGAGCGCTATCGAGGACAT
TTTATTCTCGAAACTGGTTACATCTGGGCTCGGAACTGTGGACGCCGATTACAAGAAGTGCA
CCAAGGGCCTAAGCATCGCCGACCTCGCCTGTGCTCAGTACTACAACGGAATTATGGTGCTG
CCAGGTGTCGCTGACGCAGAGCGGATGGCTATGTATACCGGCAGTCTCATTGGCGGGATTGC
GTTGGGCGGCCTGACGTCCGCTGTCTCCATCCCTTTCTCTCTGGCTATACAAGCCCGACTGA
ATTATGTGGCCCTGCAGACTGATGTCCTGCAAGAAAATCAGAAGATTCTTGCCGCCAGCTTC
AACAAGGCCATGACTAATATTGTGGATGCGTTTACCGGAGTGAATGACGCCATCACCCAAAC
GTCCCAAGCCCTGCAGACAGTCGCCACGGCGTTAAACAAAATCCAGGATGTAGTGAATCAGC
AAGGGAACAGCTTGAATCACCTGACGTCCCAGTTAAGACAGAACTTTCAGGCAATCAGTAGC
TCAATCCAGGCTATCTACGATCGATTAGATCCTCCTCAGGCAGATCAGCAGGTGGATCGGCT
CATCACCGGCCGCCTCGCGGCATTGAATGTTTTCGTAAGTCATACCTTGACCAAGTACACGG
AGGTGAGGGCCAGTCGCCAGCTGGCTCAGCAAAAAGTGAATGAGTGTGTGAAATCACAGAGC
AAACGGTACGGGTTTTGTGGAAATGGGACGCACATCTTTAGCATCGTTAATGCTGCCCCCGA
AGGGTTAGTCTTCCTGCACACTGTGCTCCTTCCTACCCAGTATAAAGATGTCGAAGCATGGT
CTGGGCTCTGTGTCGATGGAACTAACGGTTATGTCCTTCGACAGCCAAACCTCGCTCTCTAT
AAAGAAGGGAATTACTATAGGATCACCTCAAGAATCATGTTCGAGCCCAGGATACCAACAAT
GGCCGATTTTGTGCAGATTGAAAATTGTAACGTGACCTTTGTGAATATCAGTCGATCCGAGC
TTCAAACGATTGTTCCTGAGTACATCGACGTGAATAAAACTCTACAAGAGCTGTCCTATAAA
CTGCCTAATTATACCGTGCCTGACCTTGTAGTCGAGCAATACAACCAGACTATTCTGAACCT
GACATCGGAAATCTCTACATTGGAGAATAAAAGCGCCGAGCTCAATTACACAGTGCAGAAGC
TGCAGACCCTGATCGACAATATTAACAGCACTCTTGTGGACTTAAAGTGGCTGAACCGTGTG
GAGACTTACATCAAGTGGCCCTGGTGGGTGTGGCTCTGTATTTCCGTGGTCCTTATATTTGT
TGTAAGTATGCTGCTCCTGagcttctggatgtgctctaatgggtctctacagtgtagaatat
gtattTGA
PDI-229E -wtTMCT-AA
(SEQ ID NO: 153)
MAKNVAIFGLLFSLLVLVPSQIFAQTTNGLNTSYSVCNGCVGYSENVFAVESGGYIPSDFAF
NNWFLLTNTSSVVDGVVRSFQPLLLNCLWSVSGLRFTTGFVYFNGTGRGDCKGFSSDVLSDV
IRYNLNFEENLRRGTILFKTSYGVVVFYCTNNTLVSGDAHIPFGTVLGNFYCFVNTTIGNET
TSAFVGALPKTVREFVISRTGHFYINGYRYFTLGNVEAVNFNVTTAETTDFCTVALASYADV
LVNVSQTSIANIIYCNSVINRLRCDQLSFDVPDGFYSTSPIQSVELPVSIVSLPVYHKHTFI
VLYVDFKPQSGGGKCFNCYPAGVNITLANFNETKGPLCVDTSHFTTKYVAVYANVGRWSASI
NTGNCPFSFGKVNNFVKFGSVCFSLKDIPGGCAMPIVANWAYSKYYTIGSLYVSWSDGDGIT
GVPQPVEGVSSFMNVTLDKCTKYNIYDVSGVGVIRVSNDTFLNGITYTSTSGNLLGFKDVTK
GTIYSITPCNPPDQLVVYQQAVVGAMLSENFTSYGFSNVVELPKFFYASNGTYNCTDAVLTY
SSFGVCADGSIIAVQPANVSYDSVSAIVTANLSIPSNWTTSVQVEYLQITSTPIVVDCSTYV
CNGNVRCVELLKQYTSACKTIEDALRNSARLESADVSEMLTFDKKAFTLANVSSFGDYNLSS
VIPSLPTSGSRVAGRSAIEDILFSKLVTSGLGTVDADYKKCTKGLSIADLACAQYYNGIMVL
PGVADAERMAMYTGSLIGGIALGGLTSAVSIPFSLAIQARLNYVALQTDVLQENQKILAASF
NKAMTNIVDAFTGVNDAITQTSQALQTVATALNKIQDVVNQQGNSLNHLTSQLRONFQAISS
SIQAIYDRLDPPQADQQVDRLITGRLAALNVFVSHTLTKYTEVRASRQLAQQKVNECVKSQS
KRYGFCGNGTHIFSIVNAAPEGLVFLHTVLLPTQYKDVEAWSGLCVDGTNGYVLROPNLALY
KEGNYYRITSRIMFEPRIPTMADFVQIENCNVTFVNISRSELQTIVPEYIDVNKTLQELSYK
LPNYTVPDLVVEQYNQTILNLTSEISTLENKSAELNYTVQKLQTLIDNINSTLVDLKWLNRV
ETYIKWPWWVWLCISVVLIFVVSMLLLCCCSTGCCGFFSCFASSIRGCCESTKLPYYDVEKI
HIQ
PDI-229E -H5iTMCT-AA
(SEQ ID NO: 154)
MAKNVAIFGLLFSLLVLVPSQIFAQTTNGLNTSYSVCNGCVGYSENVFAVESGGYIPSDFAF
NNWFLLTNTSSVVDGVVRSFQPLLLNCLWSVSGLRFTTGFVYFNGTGRGDCKGFSSDVLSDV
IRYNLNFEENLRRGTILFKTSYGVVVFYCTNNTLVSGDAHIPFGTVLGNFYCFVNTTIGNET
TSAFVGALPKTVREFVISRTGHFYINGYRYFTLGNVEAVNFNVTTAETTDFCTVALASYADV
LVNVSQTSIANIIYCNSVINRLRCDQLSFDVPDGFYSTSPIQSVELPVSIVSLPVYHKHTFI
VLYVDFKPQSGGGKCFNCYPAGVNITLANFNETKGPLCVDTSHFTTKYVAVYANVGRWSASI
NTGNCPFSFGKVNNFVKFGSVCFSLKDIPGGCAMPIVANWAYSKYYTIGSLYVSWSDGDGIT
GVPQPVEGVSSFMNVTLDKCTKYNIYDVSGVGVIRVSNDTFLNGITYTSTSGNLLGFKDVTK
GTIYSITPCNPPDQLVVYQQAVVGAMLSENFTSYGFSNVVELPKFFYASNGTYNCTDAVLTY
SSFGVCADGSIIAVQPANVSYDSVSAIVTANLSIPSNWTTSVQVEYLQITSTPIVVDCSTYV
CNGNVRCVELLKQYTSACKTIEDALRNSARLESADVSEMLTFDKKAFTLANVSSFGDYNLSS
VIPSLPTSGSRVAGRSAIEDILFSKLVTSGLGTVDADYKKCTKGLSIADLACAQYYNGIMVL
PGVADAERMAMYTGSLIGGIALGGLTSAVSIPFSLAIQARLNYVALQTDVLQENQKILAASF
NKAMTNIVDAFTGVNDAITQTSQALQTVATALNKIQDVVNQQGNSLNHLTSQLRQNFQAISS
SIQAIYDRLDPPQADQQVDRLITGRLAALNVFVSHTLTKYTEVRASRQLAQQKVNECVKSQS
KRYGFCGNGTHIFSIVNAAPEGLVFLHTVLLPTQYKDVEAWSGLCVDGTNGYVLRQPNLALY
KEGNYYRITSRIMFEPRIPTMADFVQIENCNVTFVNISRSELQTIVPEYIDVNKTLQELSYK
LPNYTVPDLVVEQYNQTILNLTSEISTLENKSAELNYTVQKLQTLIDNINSTLVDLKWLNRV
ETYIKWPWWQILSIYSTVASSLALAIMMAGLSLWMCSNGSLQCRICI
PDI-229E -H5iCT-AA
(SEQ ID NO: 155)
MAKNVAIFGLLFSLLVLVPSQIFAQTTNGLNTSYSVCNGCVGYSENVFAVESGGYIPSDFAF
NNWFLLTNTSSVVDGVVRSFQPLLLNCLWSVSGLRFTTGFVYFNGTGRGDCKGFSSDVLSDV
IRYNLNFEENLRRGTILFKTSYGVVVFYCTNNTLVSGDAHIPFGTVLGNFYCFVNTTIGNET
TSAFVGALPKTVREFVISRTGHFYINGYRYFTLGNVEAVNFNVTTAETTDFCTVALASYADV
LVNVSQTSIANIIYCNSVINRLRCDQLSFDVPDGFYSTSPIQSVELPVSIVSLPVYHKHTFI
VLYVDFKPQSGGGKCFNCYPAGVNITLANFNETKGPLCVDTSHFTTKYVAVYANVGRWSASI
NTGNCPFSFGKVNNFVKFGSVCFSLKDIPGGCAMPIVANWAYSKYYTIGSLYVSWSDGDGIT
GVPQPVEGVSSFMNVTLDKCTKYNIYDVSGVGVIRVSNDTFLNGITYTSTSGNLLGFKDVTK
GTIYSITPCNPPDQLVVYQQAVVGAMLSENFTSYGFSNVVELPKFFYASNGTYNCTDAVLTY
SSFGVCADGSIIAVQPANVSYDSVSAIVTANLSIPSNWTTSVQVEYLQITSTPIVVDCSTYV
CNGNVRCVELLKQYTSACKTIEDALRNSARLESADVSEMLTFDKKAFTLANVSSFGDYNLSS
VIPSLPTSGSRVAGRSAIEDILFSKLVTSGLGTVDADYKKCTKGLSIADLACAQYYNGIMVL
PGVADAERMAMYTGSLIGGIALGGLTSAVSIPFSLAIQARLNYVALQTDVLQENQKILAASF
NKAMTNIVDAFTGVNDAITQTSQALQTVATALNKIQDVVNQQGNSLNHLTSQLRQNFQAISS
SIQAIYDRLDPPQADQQVDRLITGRLAALNVFVSHTLTKYTEVRASRQLAQQKVNECVKSQS
KRYGFCGNGTHIFSIVNAAPEGLVFLHTVLLPTQYKDVEAWSGLCVDGTNGYVLRQPNLALY
KEGNYYRITSRIMFEPRIPTMADFVQIENCNVTFVNISRSELQTIVPEYIDVNKTLQELSYK
LPNYTVPDLVVEQYNQTILNLTSEISTLENKSAELNYTVQKLQTLIDNINSTLVDLKWLNRV
ETYIKWPWWVWLCISVVLIFVVSMLLLSLWMCSNGSLQCRICI
PDI-229E -H5iCT(V4)-AA
(SEQ ID NO: 156)
MAKNVAIFGLLFSLLVLVPSQIFAQTTNGLNTSYSVCNGCVGYSENVFAVESGGYIPSDFAF
NNWFLLTNTSSVVDGVVRSFQPLLLNCLWSVSGLRFTTGFVYFNGTGRGDCKGFSSDVLSDV
IRYNLNFEENLRRGTILFKTSYGVVVFYCTNNTLVSGDAHIPFGTVLGNFYCFVNTTIGNET
TSAFVGALPKTVREFVISRTGHFYINGYRYFTLGNVEAVNFNVTTAETTDFCTVALASYADV
LVNVSQTSIANIIYCNSVINRLRCDQLSFDVPDGFYSTSPIQSVELPVSIVSLPVYHKHTFI
VLYVDFKPQSGGGKCFNCYPAGVNITLANFNETKGPLCVDTSHFTTKYVAVYANVGRWSASI
NTGNCPFSFGKVNNFVKFGSVCFSLKDIPGGCAMPIVANWAYSKYYTIGSLYVSWSDGDGIT
GVPQPVEGVSSFMNVTLDKCTKYNIYDVSGVGVIRVSNDTFLNGITYTSTSGNLLGFKDVTK
GTIYSITPCNPPDQLVVYQQAVVGAMLSENFTSYGFSNVVELPKFFYASNGTYNCTDAVLTY
SSFGVCADGSIIAVQPANVSYDSVSAIVTANLSIPSNWTTSVQVEYLQITSTPIVVDCSTYV
CNGNVRCVELLKQYTSACKTIEDALRNSARLESADVSEMLTFDKKAFTLANVSSFGDYNLSS
VIPSLPTSGSRVAGRSAIEDILFSKLVTSGLGTVDADYKKCTKGLSIADLACAQYYNGIMVL
PGVADAERMAMYTGSLIGGIALGGLTSAVSIPFSLAIQARLNYVALQTDVLQENQKILAASF
NKAMTNIVDAFTGVNDAITQTSQALQTVATALNKIQDVVNQQGNSLNHLTSQLRQNFQAISS
SIQAIYDRLDPPQADQQVDRLITGRLAALNVFVSHTLTKYTEVRASROLAQQKVNECVKSQS
KRYGFCGNGTHIFSIVNAAPEGLVFLHTVLLPTQYKDVEAWSGLCVDGTNGYVLRQPNLALY
KEGNYYRITSRIMFEPRIPTMADFVQIENCNVTFVNISRSELQTIVPEYIDVNKTLQELSYK
LPNYTVPDLVVEQYNQTILNLTSEISTLENKSAELNYTVQKLQTLIDNINSTLVDLKWLNRV
ETYIKWPWWVWLCISVVLIFVVSMLLLCCSNGSLQCRICI
PDI-229E-H1cCT-AA
(SEQ ID NO: 157)
MAKNVAIFGLLFSLLVLVPSQIFAQTTNGLNTSYSVCNGCVGYSENVFAVESGGYIPSDFAF
NNWFLLTNTSSVVDGVVRSFQPLLLNCLWSVSGLRFTTGFVYFNGTGRGDCKGFSSDVLSDV
IRYNLNFEENLRRGTILFKTSYGVVVFYCTNNTLVSGDAHIPFGTVLGNFYCFVNTTIGNET
TSAFVGALPKTVREFVISRTGHFYINGYRYFTLGNVEAVNFNVTTAETTDFCTVALASYADV
LVNVSQTSIANIIYCNSVINRLRCDQLSFDVPDGFYSTSPIQSVELPVSIVSLPVYHKHTFI
VLYVDFKPQSGGGKCFNCYPAGVNITLANFNETKGPLCVDTSHFTTKYVAVYANVGRWSASI
NTGNCPFSFGKVNNFVKFGSVCFSLKDIPGGCAMPIVANWAYSKYYTIGSLYVSWSDGDGIT
GVPQPVEGVSSFMNVTLDKCTKYNIYDVSGVGVIRVSNDTFLNGITYTSTSGNLLGFKDVTK
GTIYSITPCNPPDQLVVYQQAVVGAMLSENFTSYGFSNVVELPKFFYASNGTYNCTDAVLTY
SSFGVCADGSIIAVQPANVSYDSVSAIVTANLSIPSNWTTSVQVEYLQITSTPIVVDCSTYV
CNGNVRCVELLKQYTSACKTIEDALRNSARLESADVSEMLTFDKKAFTLANVSSFGDYNLSS
VIPSLPTSGSRVAGRSAIEDILFSKLVTSGLGTVDADYKKCTKGLSIADLACAQYYNGIMVL
PGVADAERMAMYTGSLIGGIALGGLTSAVSIPFSLAIQARLNYVALQTDVLQENQKILAASF
NKAMTNIVDAFTGVNDAITQTSQALQTVATALNKIQDVVNQQGNSLNHLTSQLRQNFQAISS
SIQAIYDRLDPPQADQQVDRLITGRLAALNVFVSHTLTKYTEVRASROLAQQKVNECVKSQS
KRYGFCGNGTHIFSIVNAAPEGLVFLHTVLLPTQYKDVEAWSGLCVDGTNGYVLROPNLALY
KEGNYYRITSRIMFEPRIPTMADFVQIENCNVTFVNISRSELQTIVPEYIDVNKTLQELSYK
LPNYTVPDLVVEQYNQTILNLTSEISTLENKSAELNYTVQKLQTLIDNINSTLVDLKWLNRV
ETYIKWPWWVWLCISVVLIFVVSMLLLSFWMCSNGSLQCRICI
Native OC43-CoV S protein wtTM/CT AA (AVR40344)
(SEQ ID NO: 158)
MFLILLISLPTAFAVIGDLNCTLDPRLKGSFNNRDTGPPSISIDTVDVINGLGTYYVLDRVY
LNTTLFLNGYYPTSGSTYRNMALKGTDLLSTLWFKPPFLSDFINGIFAKVKNTKVFKDGVMY
SEFPAITIGSTFVNTSYSVVVQPRTINSTQDGVNKLQGLLEVSVCQYNMCEYPHTICHPNLG
NHFKELWHYDTGVVSCLYKRNFTYDVNATYLYFHFYQEGGTFYAYFTDTGFVTKFLFNVYLG
MALSHYYVMPLTCIRRPKDGFSLEYWVTPLTPRQYLLAFNQDGIIFNAVDCMSDFMSEIKCK
TQSIAPPTGVYELNGYTVQPVADVYRRKPDLPNCNIEAWLNDKSVPSPLNWERKTFSNCNFN
MSSLMSFIQADSFTCNNIDAAKIYGMCFSSITIDKFAIPNRRKVDLQLGNLGYLQSSNYRID
TTATSCQLYYNLPAANVSVSRFNPSTWNKRFGFIEDSVFVPQPTGVFTNHSVVYAQHCFKAP
KNFCPCSSCSCPGKNNGIGTCPAGTNSLTCDNLCTLDPITLKAPDTYKCPQSKSLVGIGEHC
SGLAVKSDYCGNNSCTCQPQAFLGWSADSCLQGDKCNIFANFILHDVNNGLTCSTDLQKANT
EIELGVCVNYDLYGISGQGIFVEVNATYYNSWQNLLYDSNGNLYGFRDYITNRTFMIHSCYS
GRVSAAYHANSSEPALLFRNIKCNYVFNNSLTRQLQPINYSFDSYLGCVVNAYNSTAISVQT
CDLTVGSGYCVDYSKNRRSRRAITTGYRFTNFEPFTVNSVNDSLEPVGGLYEIQIPSEFTIG
NMEEFIQTSSPKVTIDCAAFVCGDYAACKLQLVEYGSFCDNINAILTEVNELLDTTQLQVAN
SLMNGVTLSTKLKDGVNFNVDDINFSPVLGCLGSECSKASSRSAIEDLLFDKVKLSDVGFVE
AYNNCTGGAEIRDLICVQSYKGIKVLPPLLSENQISGYTLAATSASLFPPWTAAAGVPFYLN
VQYRLNGLGVTMDVLSQNQKLIANAFNNALHAIQQGFDATNSALVKIQAVVNANAEALNNLL
QQLSNRFGAISASLQEILSRLDALEAEAQIDRLINGRLTALNAYVSQQLSDSTLVKFSAAQA
MEKVNECVKSQSSRINFCGNGNHIISLVQNAPYGLYFIHFNYVPTKYVTAKVSPGLCIAGNR
GIAPKSGYFVNVNNTWMYTGSGYYYPEPITENNVVVMSTCAVNYTKAPYVMLNTSIPNLPDF
KEELDQWFKNQTSVAPDLSLDYINVTFLDLQVEMNRLQEAIKVLNHSYINLKDIGTYEYYVK
WPWYVWLLICLAGVAMLVLLFFICCCTGCGTSCFKKCGGCCDDYTGYQELVIKTSHDD
Native 229E S protein wtTM/CT AA (P15423)
(SEQ ID NO: 159)
MFVLLVAYALLHIAGCQTTNGLNTSYSVCNGCVGYSENVFAVESGGYIPSDFAFNNWFLLTN
TSSVVDGVVRSFQPLLLNCLWSVSGLRFTTGFVYFNGTGRGDCKGFSSDVLSDVIRYNLNFE
ENLRRGTILFKTSYGVVVFYCTNNTLVSGDAHIPFGTVLGNFYCFVNTTIGNETTSAFVGAL
PKTVREFVISRTGHFYINGYRYFTLGNVEAVNFNVTTAETTDFCTVALASYADVLVNVSQTS
IANIIYCNSVINRLRCDQLSFDVPDGFYSTSPIQSVELPVSIVSLPVYHKHTFIVLYVDFKP
QSGGGKCFNCYPAGVNITLANFNETKGPLCVDTSHFTTKYVAVYANVGRWSASINTGNCPFS
FGKVNNFVKFGSVCFSLKDIPGGCAMPIVANWAYSKYYTIGSLYVSWSDGDGITGVPQPVEG
VSSFMNVTLDKCTKYNIYDVSGVGVIRVSNDTFLNGITYTSTSGNLLGFKDVTKGTIYSITP
CNPPDQLVVYQQAVVGAMLSENFTSYGFSNVVELPKFFYASNGTYNCTDAVLTYSSFGVCAD
GSIIAVQPRNVSYDSVSAIVTANLSIPSNWTTSVQVEYLQITSTPIVVDCSTYVCNGNVRCV
ELLKQYTSACKTIEDALRNSARLESADVSEMLTFDKKAFTLANVSSFGDYNLSSVIPSLPTS
GSRVAGRSAIEDILFSKLVTSGLGTVDADYKKCTKGLSIADLACAQYYNGIMVLPGVADAER
MAMYTGSLIGGIALGGLTSAVSIPFSLAIQARLNYVALQTDVLQENQKILAASFNKAMTNIV
DAFTGVNDAITQTSQALQTVATALNKIQDVVNQQGNSLNHLTSQLRQNFQAISSSIQAIYDR
LDTIQADQQVDRLITGRLAALNVFVSHTLTKYTEVRASRQLAQQKVNECVKSQSKRYGFCGN
GTHIFSIVNAAPEGLVFLHTVLLPTQYKDVEAWSGLCVDGTNGYVLRQPNLALYKEGNYYRI
TSRIMFEPRIPTMADFVQIENCNVTFVNISRSELQTIVPEYIDVNKTLQELSYKLPNYTVPD
LVVEQYNQTILNLTSEISTLENKSAELNYTVQKLQTLIDNINSTLVDLKWLNRVETYIKWPW
WVWLCISVVLIFVVSMLLLCCCSTGCCGFFSCFASSIRGCCESTKLPYYDVEKIHIQ
Native OC43-CoV S protein wtTM/CT AA (AVR40344) without signal peptide
(SEQ ID NO: 160)
VIGDLNCTLDPRLKGSFNNRDTGPPSISIDTVDVTNGLGTYYVLDRVYLNTTLFLNGYYPTS
GSTYRNMALKGTDLLSTLWFKPPFLSDFINGIFAKVKNTKVFKDGVMYSEFPAITIGSTFVN
TSYSVVVQPRTINSTQDGVNKLQGLLEVSVCQYNMCEYPHTICHPNLGNHFKELWHYDTGVV
SCLYKRNFTYDVNATYLYFHFYQEGGTFYAYFTDTGFVTKFLFNVYLGMALSHYYVMPLTCI
RRPKDGFSLEYWVTPLTPRQYLLAFNQDGIIFNAVDCMSDFMSEIKCKTQSIAPPTGVYELN
GYTVQPVADVYRRKPDLPNCNIEAWLNDKSVPSPLNWERKTFSNCNFNMSSLMSFIQADSFT
CNNIDAAKIYGMCFSSITIDKFAIPNRRKVDLQLGNLGYLQSSNYRIDTTATSCQLYYNLPA
ANVSVSRFNPSTWNKRFGFIEDSVFVPQPTGVFTNHSVVYAQHCFKAPKNFCPCSSCSCPGK
NNGIGTCPAGTNSLTCDNLCTLDPITLKAPDTYKCPQSKSLVGIGEHCSGLAVKSDYCGNNS
CTCQPQAFLGWSADSCLQGDKCNIFANFILHDVNNGLTCSTDLQKANTEIELGVCVNYDLYG
ISGQGIFVEVNATYYNSWQNLLYDSNGNLYGFRDYITNRTFMIHSCYSGRVSAAYHANSSEP
ALLFRNIKCNYVFNNSLTRQLQPINYSFDSYLGCVVNAYNSTAISVQTCDLTVGSGYCVDYS
KNRRSRRAITTGYRFTNFEPFTVNSVNDSLEPVGGLYEIQIPSEFTIGNMEEFIQTSSPKVT
IDCAAFVCGDYAACKLQLVEYGSFCDNINAILTEVNELLDTTQLQVANSLMNGVTLSTKLKD
GVNFNVDDINFSPVLGCLGSECSKASSRSAIEDLLFDKVKLSDVGFVEAYNNCTGGAEIRDL
ICVQSYKGIKVLPPLLSENQISGYTLAATSASLFPPWTAAAGVPFYLNVQYRLNGLGVTMDV
LSQNQKLIANAFNNALHAIQQGFDATNSALVKIQAVVNANAEALNNLLQQLSNRFGAISASL
QEILSRLDALEAEAQIDRLINGRLTALNAYVSQQLSDSTLVKFSAAQAMEKVNECVKSQSSR
INFCGNGNHIISLVQNAPYGLYFIHFNYVPTKYVTAKVSPGLCIAGNRGIAPKSGYFVNVNN
TWMYTGSGYYYPEPITENNVVVMSTCAVNYTKAPYVMLNTSIPNLPDFKEELDQWFKNQTSV
APDLSLDYINVTFLDLQVEMNRLQEAIKVLNHSYINLKDIGTYEYYVKWPWYVWLLICLAGV
AMLVLLFFICCCTGCGTSCFKKCGGCCDDYTGYQELVIKTSHDD
Native 229E S protein wtTM/CT AA (P15423) without signal peptide
(SEQ ID NO: 161)
QTTNGLNTSYSVCNGCVGYSENVFAVESGGYIPSDFAFNNWFLLTNTSSVVDGVVRSFQPLL
LNCLWSVSGLRFTTGFVYFNGTGRGDCKGFSSDVLSDVIRYNLNFEENLRRGTILFKTSYGV
VVFYCTNNTLVSGDAHIPFGTVLGNFYCFVNTTIGNETTSAFVGALPKTVREFVISRTGHFY
INGYRYFTLGNVEAVNFNVTTAETTDFCTVALASYADVLVNVSQTSIANIIYCNSVINRLRC
DQLSFDVPDGFYSTSPIQSVELPVSIVSLPVYHKHTFIVLYVDFKPQSGGGKCFNCYPAGVN
ITLANFNETKGPLCVDTSHFTTKYVAVYANVGRWSASINTGNCPFSFGKVNNFVKFGSVCFS
LKDIPGGCAMPIVANWAYSKYYTIGSLYVSWSDGDGITGVPQPVEGVSSFMNVTLDKCTKYN
IYDVSGVGVIRVSNDTFLNGITYTSTSGNLLGFKDVTKGTIYSITPCNPPDQLVVYQQAVVG
AMLSENFTSYGFSNVVELPKFFYASNGTYNCTDAVLTYSSFGVCADGSIIAVQPRNVSYDSV
SAIVTANLSIPSNWTTSVQVEYLQITSTPIVVDCSTYVCNGNVRCVELLKQYTSACKTIEDA
LRNSARLESADVSEMLTFDKKAFTLANVSSFGDYNLSSVIPSLPTSGSRVAGRSAIEDILFS
KLVTSGLGTVDADYKKCTKGLSIADLACAQYYNGIMVLPGVADAERMAMYTGSLIGGIALGG
LTSAVSIPFSLAIQARLNYVALQTDVLQENQKILAASENKAMTNIVDAFTGVNDAITQTSQA
LQTVATALNKIQDVVNQQGNSLNHLTSQLRQNFQAISSSIQAIYDRLDTIQADQQVDRLITG
RLAALNVFVSHTLTKYTEVRASRQLAQQKVNECVKSQSKRYGFCGNGTHIFSIVNAAPEGLV
FLHTVLLPTQYKDVEAWSGLCVDGTNGYVLRQPNLALYKEGNYYRITSRIMFEPRIPTMADF
VQIENCNVTFVNISRSELQTIVPEYIDVNKTLQELSYKLPNYTVPDLVVEQYNQTILNLTSE
ISTLENKSAELNYTVQKLQTLIDNINSTLVDLKWLNRVETYIKWPWWVWLCISVVLIFVVSM
LLLCCCSTGCCGFFSCFASSIRGCCESTKLPYYDVEKIHIQ
TMCT region of modified PDI-OC43-COV wtTMCT-AA
(SEQ ID NO: 162)
WYVWLLICLAGVAMLVLLFFICCCTGCGTSCFKKCGGCCDDYTGYQELVIKTSHDD
TMCT region of modified PDI-OC43-COV H5iTMCT-AA
(SEQ ID NO: 163)
WYQILSIYSTVASSLALAIMMAGLSLWMCSNGSLQCRICI
TMCT region of modified PDI-OC43-COV H5iCT-AA
(SEQ ID NO: 164)
WYVWLLICLAGVAMLVLLFFISLWMCSNGSLQCRICI
TMCT region of modified PDI-OC43-COV H5iCT(V4)-AA
(SEQ ID NO: 165)
WYVWLLICLAGVAMLVLLFFICCSNGSLQCRICI
TMCT region of modified PDI-OC43-COV HIcCT-AA
(SEQ ID NO: 166)
WYVWLLICLAGVAMLVLLFFISFWMCSNGSLQCRICI
TMCT region of modified PDI-229E-wtTMCT-AA
(SEQ ID NO: 167)
WWVWLCISVVLIFVVSMLLLCCCSTGCCGFFSCFASSIRGCCESTKLPYYDVEKIHIQ
TMCT region of modified PDI-229E-H5iTMCT-AA
(SEQ ID NO: 168)
WWQILSIYSTVASSLALAIMMAGLSLWMCSNGSLQCRICI
TMCT region of modified PDI-229E-H5iCT-AA
(SEQ ID NO: 169)
WWVWLCISVVLIFVVSMLLLSLWMCSNGSLQCRICI
TMCT region of modified PDI-229E-H5iCT(V4)-AA
(SEQ ID NO: 170)
WWVWLCISVVLIFVVSMLLLCCSNGSLQCRICI
TMCT region of modified PDI-229E-H1cCT-AA
(SEQ ID NO: 171)
WWVWLCISVVLIFVVSMLLLSFWMCSNGSLQCRICI
TM/CT Region of Modified OC43-CoV S protein with intervening peptide sequence Xₙ
(SEQ ID NO: 172)
WYVWLLICLAGVAMLVLLFFI - (X)n - CSNGSXXCXICI
TM/CT Region of Modified OC43-CoV S protein with intervening peptide sequence Xₙ
(SEQ ID NO: 173)
WWVWLCISVVLIFVVSMLLL - (X)n - CSNGSXXCXICI

All citations are hereby incorporated by reference.

The present invention has been described with regard to one or more embodiments. However, it will be apparent to persons skilled in the art that a number of variations and modifications can be made without departing from the scope of the invention as defined in the claims.

1. A modified coronavirus S-protein comprising, in series,
an ectodomain derived from a coronavirus S-protein,
a transmembrane and cytosolic tail domain (TMCT), wherein the TMCT is a chimeric TMCT, comprising:
a transmembrane domain (TM), wherein the TM or a portion of the TM is derived from a coronavirus S-protein and
a cytosolic tail (CT), wherein the CT or a portion of the CT is derived from an influenza hemagglutinin (HA) protein.

2. The modified coronavirus S-protein of claim 1, wherein the TM is directly fused to the CT.

3. The modified coronavirus S-protein of claim 1, wherein the TM is a chimeric TM comprising a N-terminal sequence derived from the coronavirus S-protein TM and a C-terminal sequence derived from the influenza HA protein TM.

4. The modified coronavirus S-protein of claim 3, wherein the chimeric TM comprises a N-terminal sequence derived from the coronavirus S-protein TM comprising at least 20 amino acids corresponding to amino acids 1-20 of SEQ ID NO: 18 or SEQ ID NO: 169, or at least 21 amino acids corresponding to amino acids 1-21 of SEQ ID NO: 118 or 164, or at least 22 amino acids corresponding to amino acids 1-22 of SEQ ID NO: 123 and one or more than one amino acid from the C-terminal end of the influenza HA protein TM.

5. The modified coronavirus S-protein of claim 4, wherein the one or more than one amino acid from the C-terminal end of the influenza HA protein TM are selected from AGL or conserved substitution of AGL, MAGL or conserved substitution of MAGL.

6. The modified coronavirus S-protein of claim 1, wherein the CT is a chimeric CT comprising a N-terminal sequence derived from the coronavirus S-protein CT and a C-terminal sequence derived from the influenza HA protein CT.

7. The modified coronavirus S-protein of claim 6, wherein the chimeric CT comprises a C-terminal sequence derived from the influenza HA protein CT comprising amino acids corresponding to amino acids 27-37 of SEQ ID NO: 18, 126, 128, 129, 130 or 131; or amino acids corresponding to amino acids 27-36 of SEQ ID NO: 127 and one or more than one amino acid from the N-terminal end of the coronavirus S-protein CT.

8. The modified coronavirus S-protein of claim 7, wherein the one or more than one amino acid from the N-terminal end of the coronavirus S-protein CT are selected from C or a conserved substitution of C, CC or a conserved substitution of CC, or CCM or a or a conserved substitution of CCM.

9. The modified coronavirus S-protein of claim 3, wherein the chimeric TM comprises amino acids corresponding to amino acids 1-20 of SEQ ID NO: 18 or SEQ ID NO: 169, or to amino acids 1-21 of SEQ ID NO: 118 or SEQ ID NO: 164, or amino acids 1-22 of SEQ ID NO: 123.

10. The modified coronavirus S-protein of claim 4, wherein the chimeric CT comprises amino acids corresponding to amino acids of 27-37 of SEQ ID NO: 18, 126, 128, 129, 130 or 131; or amino acids 27-36 of SEQ ID NO: 127.

11. The modified coronavirus S-protein of claim 1, wherein the chimeric TMCT comprises a chimeric TM comprising amino acids corresponding to amino acids 1-20 of SEQ ID NO: 18 or SEQ ID NO: 169, or to amino acids 1-21 of SEQ ID NO: 118 or SEQ ID NO: 164, or amino acids 1-22 of SEQ ID NO: 123 and, a chimeric CT comprising amino acids corresponding to amino acids 27-37 of SEQ ID NO: 18, 126, 128, 129, 130 or 131; amino acids 27-36 of SEQ ID NO: 127 or a combination thereof.

12. The modified S-protein of claim 1, wherein the S-protein comprises one or more than one amino acid substitution when compared to a wild-type coronavirus S-protein amino acid sequence.

13-20. (canceled)

21. The modified S-protein of claim 12, wherein the one or more than one substitution maintains the S-protein in a pre-fusion state or produces are higher yield of the modified S-protein when expressed in a host or host cell, when compared to the yield of a corresponding S-protein without the one or more than one substitutions expressed in the host or host cell.

22.-25. (canceled)

26. The modified S-protein of claim 1, wherein the TMCT comprises a sequence having about 80% to about 100% identity with the sequence of SEQ ID NO: 18, 19, 37, 38, 39, 64, 126, 127, 128, 129, 130, 131, 118, 119, 120, 123, 124, 125, 134, 135, 164, 165, 166, 169, 170, 171, 172 or 173.

27.-32. (canceled)

33. The modified S-protein of claim 1, wherein the S-protein is produced as a precursor, the precursor protein comprising from 80% to 100% identity with amino acids 1-1234 of SEQ ID 1, or with amino acids 1-1234 of SEQ ID NO: 5, with amino acids 1-1243 of SEQ ID NO: 30, with amino acids 1-1227 of SEQ ID NO: 95, with amino acids 1-1325 of SEQ ID NO: 108, with amino acids 1-1216 of SEQ ID NO: 112, with amino acids 1-1318 of SEQ ID NO: 113, with amino acids 1-1335 of SEQ ID NO: 144, with amino acids 1-1143 of SEQ ID NO: 155, with amino acids 1-1325 of SEQ ID NO: 158, with amino acids 1-1135 of SEQ ID NO: 159, and wherein the amino acid sequence of the CT comprises from 80% to 100% identity with the sequence of SEQ ID NO: 15, or with amino acids 35-50 of SEQ ID NO 6, 8, 7, 9, 10, 12, 13 or 14, or with amino acids 34-49 of SEQ ID NO 11, or with amino acids 553-568 of SEQ ID NO:3.

34. A nucleic acid comprising a nucleotide sequence encoding the modified S protein of claim 1.

35. (canceled)

36. A virus like particle (VLP) comprising the modified S-protein of claim 1.

37.-38. (canceled)

39. A vaccine for inducing an immune response, the vaccine comprising an effective dose of the modified S-protein of claim 1.

40. (canceled)

41. A method for inducing immunity to a Coronavirus infection in a subject, the method comprising administering the vaccine of claim 39 to the subject.

42.-45. (canceled)

46. A non-human host or host cell comprising the modified S-protein of claim 1.

47. (canceled)

48. A method of producing a virus like particle (VLP) in a non-human host or host cell comprising:
a) introducing the nucleic acid of claim 34 into the non-human host or host cell, or providing the non-human host or host cell comprising the nucleic acid of claim 34, and
b) incubating the non-human host or host cell under conditions that permit the expression of the nucleic acid, thereby producing the VLP.

49. The method of claim 47, the method further comprising step c) of harvesting the non-human host or host cell.

50-56. (canceled)

57. A composition comprising a virus-like particles (VLP), the VLP comprising a modified coronavirus S-protein comprising, in series, an ectodomain derived from a coronavirus S-protein, a transmembrane and cytosolic tail domain (TMCT), wherein the TMCT is a chimeric TMCT, comprising: a transmembrane domain (TM), wherein the TM or a portion of the TM is derived from a coronavirus S-protein and a cytosolic tail (CT), wherein the CT or a portion of the CT is derived from an influenza hemagglutinin (HA) protein and wherein the S-protein comprises substitutions at positions 667, 668, 670, 971 and 972 when compared to reference amino acid sequence of SEQ ID NO: 2.

58. A composition comprising a virus-like particle (VLP), the VLP comprising a modified coronavirus S-protein comprising, in series, an ectodomain derived from a coronavirus S-protein, a transmembrane and cytosolic tail domain (TMCT), wherein the TMCT is a chimeric TMCT, comprising: a transmembrane domain (TM), wherein the TM or a portion of the TM is derived from a coronavirus S-protein and a cytosolic tail (CT), wherein the CT or a portion of the CT is derived from an influenza hemagglutinin (HA) protein and wherein the S-protein comprises a glycine substitution at position 667, a serine substitution at position 668, a serine substitution at position 670, a proline substitution at position 971 and a proline substitution at position 972, the position corresponding to reference amino acid sequence of SEQ ID NO: 2.

59. A composition comprising a virus-like particle (VLP), the VLP comprising a modified coronavirus S-protein, the modified S-protein comprising the sequence of SEQ ID NO: 21 or amino acids 25-1259 of SEQ ID NO: 51.

60. (canceled)
